(19) United States
(12) Patent Application Publication
(10) Pub. No.: US 2026/0162067 A1
(43) Pub. Date: Jun. 11, 2026

(54) SYSTEMS AND METHODS FOR CONFIRMING MEETING EVENTS USING ELECTRONIC ACTIVITIES

(71) Applicant: People ai, Inc., San Francisco, CA (US)

(72) Inventors: Oleg Rogyskyy, San Francisco, CA (US); John Wulf, San Francisco, CA (US); Sathya Hariesh Prakash, San Francisco, CA (US); Tetiana Lutsaievska, San Francisco, CA (US)

(73) Assignee: People ai, Inc., San Francisco, CA (US)

(21) Appl. No.: 19/448,003

(22) Filed: Jan. 13, 2026

Related U.S. Application Data
(63) Continuation of application No. 18/939,356, filed on Nov. 6, 2024, now abandoned.
Application No. 18/939,356 is a continuation of application No. 18/324,775, filed on May 26, 2023, now Pat. No. 12,166,832.
Application No. 18/324,775 is a continuation of application No. 17/102,397, filed on Nov. 23, 2020, now Pat. No. 11,979,468.
Application No. 17/102,397 is a continuation of application No. PCT/US2019/034062, filed on May 24, 2019.

(51) Int. Cl. G06Q 10/107; G06F 7/14; G06F 9/54; G06F 11/30; G06F 11/34; G06F 16/11; G06F 16/17; G06F 16/178; G06F 16/182; G06F 16/21; G06F 16/215; G06F 16/22; G06F 16/23; G06F 16/245; G06F 16/2455; G06F 16/2457; G06F 16/2458; G06F 16/25; G06F 16/26; G06F 16/27; G06F 16/28; G06F 16/29; G06F 16/31; G06F 16/335; G06F 16/355; G06F 16/901; G06F 16/903; G06F 16/9035; G06F 16/906; G06F 16/9535; G06F 21/62; G06F 40/20; G06F 40/205; G06F 40/237; G06F 40/295; G06N 3/08; G06N 5/025; G06N 5/04; G06N 7/02; G06N 20/00; G06Q 10/04; G06Q 10/0631; G06Q 10/0639; G06Q 10/10; G06Q 10/109; G06Q 10/1091; G06Q 10/1093; G06Q 50/22; G16H 15/00; G16H 50/20; G16H 50/30; H04L 12/14; H04L 41/14; H04L 43/00; H04L 43/026; H04L 43/045; H04L 43/062; H04L 43/065; H04L 43/067; H04L 43/08; H04L 43/0876; H04L 51/046; H04L 51/212; H04L 51/234; H04L 51/42; H04L 61/45; H04L 67/125; H04L 67/30; H04L 67/303; H04L 67/306; H04L 67/50; H04L 101/00; H04L 101/35; H04L 101/37; H04M 3/22; H04M 3/436; H04M 3/56; H04M 15/00

(52) U.S. Cl. CPC G06Q 10/107; G06F 7/14; G06F 9/542; G06F 11/3024; G06F 11/3452; G06F 11/3495; G06F 16/122; G06F 16/1734; G06F 16/178; G06F 16/182; G06F 16/212; G06F 16/215; G06F 16/219; G06F 16/22; G06F 16/221; G06F 16/2228; G06F 16/2264; G06F 16/2272; G06F 16/23; G06F 16/235; G06F 16/2358; G06F 16/2365; G06F 16/2379; G06F 16/2386; G06F 16/245; G06F 16/24558; G06F 16/24564; G06F 16/2457; G06F 16/24575; G06F 16/24578; G06F 16/2477; G06F 16/254; G06F 16/256; G06F 16/26; G06F 16/27; G06F 16/273; G06F 16/28; G06F 16/285; G06F 16/288; G06F 16/289; G06F 16/29; G06F 16/313; G06F 16/337; G06F 16/355; G06F 16/901; G06F 16/9024; G06F 16/90344; G06F 16/9035; G06F 16/906; G06F 16/9535; G06F 21/6218; G06F 21/6245; G06F 40/20; G06F 40/237; G06F 40/295; G06N 3/08; G06N 5/025; G06N 5/04; G06N 7/02; G06Q 10/04; G06Q 10/063114; G06Q 10/06312; G06Q 10/06315; G06Q 10/06393; G06Q 10/06398; G06Q 10/109; G06Q 10/1091; G06Q 10/1093; G06Q 50/22; G16H 50/20; H04L 41/14; H04L 43/026; H04L 43/045; H04L 43/062; H04L 43/065; H04L 43/067; H04L 43/0876; H04L 43/14; H04L 51/046; H04L 51/212; H04L 51/234; H04L 51/42; H04L 61/45; H04L 67/125; H04L 67/30; H04L 67/303; H04L 67/306; H04L 67/535; H04M 3/436; H04M 15/755; G06F 40/205; G06N 20/00; G06Q 10/10; G16H 15/00; G16H 50/30; H04L 12/1407; H04L 43/08; H04L 2101/00; H04L 2101/35; H04L 2101/37; H04M 3/2218; H04M 3/56

(57) ABSTRACT

The system described herein can automatically match, link, or otherwise associate electronic activities with one or more record objects. For an electronic activity that is eligible or qualifies to be matched with one or more record objects, the system can identify one or more set of rules or rule sets. Using the rule sets, the system can identify candidate record objects. The system can then rank the identified candidate record objects to select one or more record objects with which to associate the electronic activity. The system can then store an association between the electronic activity and the selected one or more record objects.

### CROSS-REFERENCE TO RELATED APPLICATIONS

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034046 (Attorney Docket No. PPL-00 IPC), filed on May 24, 2019 (Attorney Docket No. PPL-00 IPC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/371,048, filed Mar. 31, 2019, U.S. patent application Ser. No. 16/420,059, filed May 22, 2019, U.S. patent application Ser. No. 16/421,280, filed May 23, 2019, U.S. patent application Ser. No. 16/421,288, filed May 23, 2019, U.S. patent application Ser. No. 16/421,298, filed May 23, 2019, U.S. patent application Ser. No. 16/421,151, filed May 23, 2019, U.S. patent application Ser. No. 16/421,309, filed May 23, 2019, and U.S. patent application Ser. No. 16/421,328, filed May 23, 2019, each of which are incorporated herein by reference for all purposes.

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034050 (Attorney Docket No. PPL-002PC), filed on May 24, 2019 (Attorney Docket No. PPL-002PC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/371,048, filed Mar. 31, 2019, U.S. patent application Ser. No. 16/371,037, filed Mar. 31, 2019, U.S. patent application Ser. No. 16/371,041, filed Mar. 31, 2019, U.S. patent application Ser. No. 16/398,220, filed Apr. 29, 2019, U.S. patent application Ser. No. 16/399,706, filed Apr. 30, 2019, and U.S. patent application Ser. No. 16/418,629, filed May 21, 2019, each of which are incorporated herein by reference for all purposes.

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034052, filed on May 24, 2019 (Attorney Docket No. PPL-003PC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/360,960u, filed Mar. 21, 2019, U.S. patent application Ser. No. 16/360,884, filed Mar. 21, 2019, U.S. patent application Ser. No. 16/360,997, filed Mar. 21, 2019, U.S. patent application Ser. No. 16/361,009, filed Mar. 21, 2019, U.S. patent application Ser. No. 16/361,025, filed Mar. 21, 2019, U.S. patent application Ser. No. 16/371,035, filed Mar. 31, 2019, U.S. patent application Ser. No. 16/399,690, filed Apr. 30, 2019, U.S. patent application Ser. No. 16/418,539, filed May 21, 2019, and U.S. patent application Ser. No. 16/418,725, filed May 21, 2019, each of which are incorporated herein by reference for all purposes.

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034042, filed on May 24, 2019 (Attorney Docket No. PPL-004PC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/360,866, filed Mar. 21, 2019, U.S. patent application Ser. No. 16/360,933, filed Mar. 21, 2019, U.S. patent application Ser. No. 16/360,953, filed Mar. 21, 2019, U.S. patent application Ser. No. 16/398,153, filed Apr. 29, 2019, U.S. patent application Ser. No. 16/418,769, filed May 21, 2019, and U.S. patent application Ser. No. 16/418,891, filed May 21, 2019, each of which are incorporated herein by reference for all purposes.

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034045, filed on May 24, 2019 (Attorney Docket No. PPL-005PC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/371,042, filed Mar. 31, 2019, U.S. patent application Ser. No. 16/371,050, filed Mar. 31, 2019, U.S. patent application Ser. No. 16/371,044, filed Mar. 31, 2019, U.S. patent application Ser. No. 16/398,157, filed Apr. 29, 2019, U.S. patent application Ser. No. 16/399,768, filed Apr. 30, 2019, U.S. patent application Ser. No. 16/418,747, filed May 21, 2019, U.S. patent application Ser. No. 16/418,851, filed May 21, 2019, U.S. patent application Ser. No. 16/418,867, filed May 22, 2019, and U.S. patent application Ser. No. 16/420,052, filed May 22, 2019, each of which are incorporated herein by reference for all purposes.

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034030 (Attorney Docket No. PPL-006PC), filed on May 24, 2019 (Attorney Docket No. PPL-006PC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/371,048, filed Mar. 31, 2019, U.S. patent application Ser. No. 16/420,059, filed May 22, 2019, U.S. patent application Ser. No. 16/421,280, filed May 23, 2019, U.S. patent application Ser. No. 16/421,288, filed May 23, 2019, U.S. patent application Ser. No. 16/421,298, filed May 23, 2019, U.S. patent application Ser. No. 16/421,151, filed May 23, 2019, U.S. patent application Ser. No. 16/421,309, filed May 23, 2019, and U.S. patent application Ser. No. 16/421,328, filed May 23, 2019, each of which are incorporated herein by reference for all purposes.

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034070, filed on May 24, 2019 (Attorney Docket No. PPL-007PC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/398,260, filed Apr. 29, 2019, U.S. patent application Ser. No. 16/421,324, filed May 23, 2019, and U.S. patent application Ser. No. 16/421,370, filed May 23, 2019, each of which are incorporated herein by reference for all purposes.

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034033, filed on May 24, 2019 (Attorney Docket No. PPL-008PC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/399,679, filed Apr. 30, 2019, U.S. patent application Ser. No. 16/418,807, filed May 21, 2019, U.S. patent application Ser. No. 16/418,846, filed May 21, 2019, U.S. patent application Ser. No. 16/419,583, filed May 22, 2019, and U.S. patent application Ser. No. 16/420,039, filed May 22, 2019, each of which are incorporated herein by reference for all purposes.

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034068, filed on May 24, 2019 (Attorney Docket No. PPL-009PC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/360,892, filed Mar. 21, 2019, U.S. patent application Ser. No. 16/371,039, filed Mar. 31, 2019, U.S. patent application Ser. No. 16/371,049, filed Mar. 31, 2019, U.S. patent application Ser. No. 16/399,787, filed Apr. 30, 2019, U.S. patent application Ser. No. 16/400,000, filed Apr. 30, 2019, and U.S. patent application Ser. No. 16/418,826, filed May 21, 2019, each of which are incorporated herein by reference for all purposes.

The present application is a continuation of U.S. application Ser. No. 18/939,356, filed on Nov. 6, 2024, which claims priority to Ser. No. 18/324,775, filed on May 26, 2023, which claims priority to U.S. application Ser. No. 17/102,397, filed on Nov. 23, 2020, which claims priority to P.C.T. Patent Application No. PCT/US2019/034062, filed on May 24, 2019 (Attorney Docket No. PPL-010PC), which claims the benefit of and priority to U.S. Provisional Patent Application 62/747,452, filed Oct. 18, 2018, U.S. Provisional Patent Application 62/725,999, filed Aug. 31, 2018, U.S. Provisional Patent Application 62/676,187, filed May 24, 2018, U.S. patent application Ser. No. 16/213,754, filed Dec. 7, 2018, U.S. patent application Ser. No. 16/237,579, filed Dec. 31, 2018, U.S. patent application Ser. No. 16/237,580, filed Dec. 31, 2018, U.S. patent application Ser. No. 16/237,582, filed Dec. 31, 2018, U.S. patent application Ser. No. 16/237,585, filed Dec. 31, 2018, U.S. patent application Ser. No. 16/398,150, filed Apr. 29, 2019, U.S. patent application Ser. No. 16/418,836, filed May 21, 2019, U.S. patent application Ser. No. 16/418,892, filed May 21, 2019, and U.S. patent application Ser. No. 16/421,256, filed May 21, 2019, each of which are incorporated herein by reference for all purposes.

### BACKGROUND

An organization may attempt to manage or maintain a system of record associated with electronic communications at the organization. The system of record can include information such as contact information, logs, and other data associated with the electronic activities. Data regarding the electronic communications can be transmitted between computing devices associated with one or more organizations using one or more transmission protocols, channels, or formats, and can contain various types of information. For example, the electronic communication can include information about a sender of the electronic communication, a recipient of the electronic communication, and content of the electronic communication. The information regarding the electronic communication can be input into a record being managed or maintained by the organization. However, due to the large volume of heterogeneous electronic communications transmitted between devices and the challenges of manually entering data, inputting the information regarding each electronic communication into a system of record can be challenging, time consuming, and error prone.

### SUMMARY

The present disclosure relates to systems and methods for constructing a node graph based on electronic activity. The node graph can include a plurality of nodes and a plurality of edges between the nodes indicating activity or relationships that are derived from a plurality of data sources that can include one or more types of electronic activities. The plurality of data sources can further include systems of record, such as customer relationship management systems, enterprise resource planning systems, document management systems, applicant tracking systems or other sources of data that may maintain electronic activities, activities or records.

The present disclosure further relates to systems and methods for using the node graph to manage, maintain, improve, or otherwise modify one or more systems of record by linking and or synchronizing electronic activities to one or more record objects of the systems of record. In particular, the systems described herein can be configured to automatically synchronize real-time or near real-time electronic activity to one or more objects of systems of record. The systems can further extract business process information from the systems of record and in combination with the node graph, use the extracted business process information to improve business processes and to provide data driven solutions to improve such business processes.

At least one aspect of the present disclosure is directed to systems and methods for maintaining an electronic activity derived member node network. For example, a node profile for a member node in a node graph can include information such as first name, last name, company, and job title. However, it may be challenging to accurately and efficiently populate fields in a node profile due to large number of member nodes. Furthermore, permitting self-population of node profiles by member nodes can result in erroneous data values, improper data values, or otherwise undesired data values due in part to human bias. Having erroneous data values in a node profile can cause downstream components or functions that perform processing using the node profiles to malfunction or generate faulty outputs.

Thus, systems and methods of the present technical solution can generate an electronic activity derived member node network that includes node profiles for a member node that is generated based on electronic activity. By generating the member node profile for the member node using electronic activity and a statistical analysis, the system can generate the profile with data fields and values that pass a verification process or statistical analysis using electronic activities.

According to at least one aspect of the disclosure a method can include accessing, by one or more processors, a plurality of electronic activities that are transmitted or received via electronic accounts of one or more data source providers. The method can include accessing, by the one or more processors, a plurality of record objects of one or more systems of record. Each of the record object of the plurality of record objects can correspond to a record object type. Each of the record objects can include one or more object fields having one or more object field values. The system or record can correspond to the one or more data source providers. The method can include identifying, by the one or more processors, an electronic activity of the plurality of electronic activities to match to one or more record objects. The electronic activity of the plurality of electronic activities can identify participants that can include a sender of the electronic activity and one or more recipients of the electronic activity. The method can include determining, by the one or more processors, a data source provider associated with providing the one or more processors access to the electronic activity. The method can include identifying, by the one or more processors, a system of record corresponding to the determined data source provider. The system of record can include a plurality of candidate record objects to which to match the electronic activity. The method can include determining, by the one or more processors and responsive to applying a first policy including one or more filtering rules, that the electronic activity is to be matched to at least one record object of the identified system of record. The method can include identifying, by the one or more processors and responsive to applying a second policy including one or more rules for identifying candidate record objects based on one or more participants of the electronic activity, one or more candidate record objects to which to match the electronic activity. The method can include selecting, by the one or more processors, at least one candidate record object based on the second policy. The method can include storing, by the one or more processors, in a data structure an association between the selected at least one candidate record object and the electronic activity.

In some implementations, identifying the system of record corresponding to the determined data source provider can include identifying, by the one or more processors, the system of record based on a domain associated with an email address of the sender of the electronic activity. Determining that the electronic activity is to be matched to at least one record object of the identified system of record can include determining that the electronic activity does not satisfy one or more filtering rules configured to cause the one or more processors to restrict the electronic activity from being matched to the at least one record object. The filtering rules can include at least one of i) a keyword rule configured to restrict electronic activities including a predetermined keyword; ii) a regex pattern rule configured to restrict electronic activities including one or more character strings that match a predetermined regex pattern; iii) a logic-based rule configured to restrict electronic activities based on the participants of the electronic activities satisfying a predetermined group of participants.

The filtering rules can be defined by the data source provider of the electronic activity and the system of record to which to match the electronic activity. Determining that the electronic activity is to be matched to at least one record object of the identified system of record can include determining that the electronic activity does not include one or more predetermined words included in a list of restricted words. Determining to match the electronic activity can include determining that the electronic activity does not include any character strings that have a regular expression (regex) pattern that matches a predefined regex pattern included in a list of restricted regex patterns. Determining to match the electronic activity can include determining that the sender and at least one of the one or more recipients match a list of restricted sender-recipient pairs.

The method can include selecting candidate record objects based on a first policy and a second policy. The second policy can include a first set of rules and a second set of rules. The candidate record objects can be identified by applying the second policy including the first set of rules for identifying candidate record objects based on one or more recipients of the electronic activity. The first set of rules can identify a first set of candidate record objects to which to match the electronic activity. Each of the candidate record objects of the first set can be identified based on the one or more recipients of the electronic activity. The method can include identifying, by the one or more processors, responsive to applying the second policy including the second set of rules for identifying candidate record objects based on the sender of the electronic activity, a second set of candidate record objects to which to match the electronic activity. Each of the candidate record object of the second set can be identified based on the sender of the electronic activity. The method can include selected at least one candidate record object included in both the first set of candidate record objects and the second set of candidate record objects. The selected candidate record object can be match to the electronic activity based on the first set of rules and the second set of rules of the second policy.

The method can include identifying, responsive to applying the second policy, the first set of candidate record objects by applying a first matching rule of the first set of rules to identify one or more first record objects corresponding to a first record object type and applying a second matching rule of the first set of rules to identify one or more second record objects corresponding to a second record object type. The second policy can assign a first priority level to the first matching rule and a second priority level to the second matching rule. The first priority level can be greater than the second priority level. The method can include receiving, from the data source provider, at least one instruction to select a subset of the first set of rules or the second set of rules and assign a first priority to a first matching rule of the subset and assign a second priority level to a second matching rule of the subset.

In some implementations, the method can include determining, by the one or more processors, that a first electronic activity previously matched to a first record object of the data source provider is matched to a second record object of the data source provider. The method can include determining, by the one or more processors, one or more matching policies of the data source provider that apply to the first electronic activity. The method can include updating, by the one or more processors, responsive to determining that the first electronic activity previously matched to the first record object is matched to the second record object, the one or more matching policies for the data source provider such that the one or more processors can match a second electronic activity including the participants of the first electronic activity to the second record object. The method can include identifying one or more candidate record objects of a first record object type based on the recipients of the electronic activity.

In some implementations, the first set of rules can include a rule to identify one or more record objects of a first record object type based on an object field value of the record object that identifies one or more nodes. Identifying, responsive to applying the second policy, the first set of candidate record objects can include identifying one or more candidate record objects of the first record object type responsive to determining that the one or more of the participants are identified by the object field value.

The object field value can be a value of a first object field corresponding to an object owner that identifies an owner of the record object or a second object field corresponding to a team that identifies a group of people linked with the record object in the system of record. Identifying, responsive to applying the second policy, the first set of candidate record objects can include identifying a particular record object of the data source provider responsive to determining that the electronic activity includes a predetermined character string that satisfies a rule to match electronic activities including the character string to a particular record object. The second set of rules can include a rule to identify one or more record objects of a first record object type that are linked with the sender of the electronic activity. Identifying, responsive to applying the second policy, the second set of candidate record objects can include identifying one or more record objects that identify the sender as an object field value to an object field of the record object.

The second set of rules can include a rule to identify one or more record objects of a first record object type based on an object field value of the record object that identifies one or more nodes. Identifying, responsive to applying the second policy, the second set of candidate record objects can include identifying one or more candidate record objects of the first record object type responsive to determining that the one or more of the participants are identified by the object field value.

The method can include retrieving, from one or more second servers, a first plurality of record objects corresponding to a first system of record of a first data source provider and second plurality of record objects corresponding to a second system of record of a second data source provider. The method can include assigning, by the one or more processors, one or more tags to the electronic activity based on a first character string included in a body of the electronic activity; ii) a second character string included in a metadata of the electronic activity; or iii) other electronic activity.

The method can include identifying, by the one or more processors, at least one candidate record object of the one or more record objects based on one or more tags assigned to the electronic activity. The method can include determining, by the one or more processors, for at least one of the participants of the electronic activity, a respective unique identifier used by the system of record corresponding to the data source provider to represent the participant.

According to at least one aspect of the disclosure a method can include maintaining, by one or more processors, a plurality of node profiles. The node profiles can correspond to a plurality of unique entities. Each of the node profiles can include a plurality of fields. Each field of the plurality of fields can include one or more value data structures. Each value data structure of the one or more value data structures can include a node field value and one or more entries corresponding to respective one or more data points that support the node field value of the value data structure. The method can include accessing, by the one or more processors, a plurality of electronic activities transmitted or received via electronic accounts that can be associated with one or more data source providers. The one or more processors can be configured to update the plurality of node profiles using the plurality of electronic activities. The method can include maintaining, by the one or more processors, a plurality of record objects of one or more systems of record. Each record object of the plurality of record objects can include one or more object fields having one or more object field values. The method can include extracting, by the one or more processors, data included in an electronic activity of the plurality of electronic activities. The method can include matching, by the one or more processors, the electronic activity to at least one node profile of the plurality of node profiles based on determining that the extracted data of the electronic activity and the one or more values of the fields of the at least one node profile satisfy a node profile matching policy. The method can include matching, by the one or more processors, the electronic activity to at least one record object of the plurality of record objects based on the extracted data of the electronic activity and object values of the at least one record object. The method can include storing, by the one or more processors, in a data structure, an association between the electronic activity and the at least one record object.

In some implementations, the method can include retrieving, from one or more second processors, a first plurality of record objects corresponding to a first system of record of a first data source provider and second plurality of record objects corresponding to a second system of record of a second data source provider. The method can include identifying, by the one or more processors, a sender of the electronic activity of the plurality of electronic activities. The method can include selecting, by the one or more processors, a first node profile of the plurality of node profiles associated with the sender of the electronic activity. The method can include identifying, by the one or more processors, a first set of record objects of the plurality of record objects of the one or more systems of record based on the first node profile.

In some implementations, the method can include identifying, by the one or more processors, a recipient of the electronic activity of the plurality of electronic activities. The method can include selecting, by the one or more processors, a second node profile of the plurality of node profiles associated with the recipient of the electronic activity. The method can include identifying, by the one or more processors, a second set of record objects of the plurality of record objects of the one or more systems of record based on the second node profile. The method can include matching the electronic activity to the at least one record object of the plurality of record objects based on an intersection of the first set of record objects and the second set of record objects.

In some implementations, the method can include determining, by the one or more processors, a plurality of candidate record objects based on the intersection of the first set of record objects and the second set of record objects. The method can include identifying, by the one or more processors, each of the plurality of candidate record objects as belonging to one of a plurality of record object types. The method can include matching the electronic activity to two or more of the plurality of candidate record objects. Each of the two or more of the plurality of candidate record objects can include a different record object type.

In some implementations, matching the electronic activity to at least one record object can include matching the electronic activity to the at least one record object based on a matching policy. The matching policy can include a first set of rules for identifying candidate record objects based on one or more recipients of the electronic activity and a second set of rules for identifying candidate record objects based on the sender of the electronic activity. Matching the electronic activity to the at least one record object can include identifying, by the one or more processors, responsive to applying the policy including the first set of rules for identifying candidate record objects based on one or more recipients of the electronic activity, a first set of candidate record objects to which to match the electronic activity. Each of the candidate record objects of the first set can be identified based on the one or more recipients of the electronic activity. The method can include identifying, by the one or more processors, responsive to applying the second policy including the second set of rules for identifying candidate record objects based on the sender of the electronic activity, a second set of candidate record objects to which to match the electronic activity. Each of the candidate record object of the second set can be identified based on the sender of the electronic activity. The method can include selecting by the one or more processors, at least one candidate record object included in both the first set of candidate record objects and the second set of candidate record objects to match to the electronic activity based on the first set of rules and the second set of rules of the matching policy.

In some implementations, the method can include selecting, by the one or more processors, based on natural language processing, a string in a body of the electronic activity. The method can include selecting, by the one or more processors, a plurality of candidate record objects based on the string in the body of the electronic activity. Matching the electronic activity to at least one record object of the plurality of record objects can include matching the electronic activity to at least one of the plurality of candidate record objects. In some implementations, the method can include matching the electronic activity to two or more node profiles of the plurality of node profiles based on determining that the extracted data of the electronic activity and the one or more values of the fields of the two or more node profiles satisfy the node profile matching policy. The method can include determining, by the one or more processors, a relationship between two or more node profiles based on the one or more values of the fields of the two or more node profiles. The method can include assigning, by the one or more processors, one or more tags to the electronic activity based on the relationship between the two or more node profiles.

In some implementations, the method can include assigning, by the one or more processors, one or more tags to the electronic activity. The tags can be assigned based on i) one or more node profiles associated with a sender or one or more recipients of the electronic activity; ii) relationship between the one or more node profiles associated with the sender and the one or more recipients of the electronic activity; iii) a first string included in a body of the electronic activity; iv) a second string included in a metadata of the electronic activity; or v) other electronic activity.

In some implementations, the method can include identifying, by the one or more processors, a first subset of record objects of the plurality of record objects of the one or more systems of record based on one or more tags assigned to the electronic activity. Matching the electronic activity to at least one record object of the plurality of record objects can include matching the electronic activity to at least one of the first subset of record objects of the plurality of record objects based on the one or more tags assigned to the electronic activity.

In some implementations, the method can include identifying, by the one or more processors, a sender and one or more recipients of the electronic activity of the plurality of electronic activities. Matching the electronic activity to at least one node profile of the plurality of node profiles can include matching the electronic activity to a node profile of the sender and a node profile of each of the one or more recipients.

In some implementations, the method can include selecting, by the one or more processors, a first set of record objects of the plurality of record objects of the one or more systems of record based on a node field value of the node profile of the sender. The method can include selecting, by the one or more processors, a second set of record objects of the plurality of record objects of the one or more systems of record based on a node field value of the node profiles of each of the one or more recipients.

The method can include identifying, by the one or more processors, a subset of the plurality of electronic activities matched to a first record object of the plurality of record objects. The method can include identifying, by the one or more processors, for each electronic activity of the subset of the plurality of electronic activities matched to the first record object, one or more node profiles that are matched with the electronic activity. The method can include determining, by the one or more processors, a stage of the first record object based on the identified one or more node profiles of each of the subset of electronic activities. The method can include identifying, by the one or more processors, a subset of the plurality of electronic activities matched to a first record object of the plurality of record objects. The method can include determining, by the one or more processors, a stage of the first record object based on tags assigned to one or more electronic activities of the subset of electronic activities.

The method can include identifying, by the one or more processors, for a record object, a corresponding record object maintained by a second processor. The method can include determining, by the one or more processors, a plurality of processor assigned stages associated with the corresponding record object. Each stage of the plurality of processor assigned stages can indicate a proximity to completion of an event. The method can include mapping, by the one or more processors, each processor assigned stage of the corresponding record object to a stage of a plurality of system assigned stages of the record object. The method can include determining, by the one or more processors, for at least one data point of the one or more data points of a value of a field of the node profile, a contribution score of the data point based on a time corresponding to when the data point was generated or updated. The method can include generating, by the one or more processors, a confidence score of the value of the field of the node profile based on the contribution score of the data point.

The method can include matching, by the one or more processors, the electronic activity to the at least one node profile of the plurality of node profiles based on the confidence score of the value of the field of the at least one node profile. Determining the contribution score can include determining, for the at least one data point, a contribution score of the data point based on a trust score assigned to a source of the data point, the trust score determined based on a type of source of the data point.

According to at least one aspect of the disclosure a method can accessing, by one or more processors, a plurality of electronic activities transmitted or received via electronic accounts associated with a plurality of data source providers, the one or more processors maintaining a plurality of node profiles and configured to update the plurality of node profiles using the plurality of electronic activities. The method can include identifying, by the one or more processors, from data included in an electronic activity of the plurality of electronic activities to link to one or more node profiles, a plurality of strings. The method can include generating, by the one or more processors, a plurality of activity field-value pairs from the plurality of strings using an electronic activity parsing policy. The method can include comparing, by the one or more processors, the plurality of activity field-value pairs to respective node field value pairs of one or more node profiles maintained by the one or more processors to identify a subset of activity field value pairs that match respective node field-value pairs of the one or more node profiles. The method can include generating, by the one or more processors, for each node profile of a plurality of node profiles, a match score of the node profile indicating a likelihood that the electronic activity is transmitted or received by an account corresponding to the node profile based on comparing the plurality of activity field-value pairs to respective node field-value pairs of the node profile. The method can include determining, by the one or more processors, a subset of the plurality of node profiles with which to link the electronic activity responsive to determining that the match score of each node profile of the subset satisfies a threshold. The method can include updating, by the one or more processors, a data structure to include an association between the electronic activity and each node profile of the subset of the plurality of node profiles.

In some implementations, the method includes selecting, by the one or more processors, a first node profile of the subset of the plurality of node profiles with which to match the electronic activity based on the match score of the first node profile. The method can include linking, by the one or more processors, the electronic activity with the first node profile. In some implementations, the method includes determining, by the one or more processors, that a first value of a first activity field-value pair matches a first value of a first node field-value pair. The method can include adding, by the one or more processors, to a first value data structure of the first node field-value pair, a first entry identifying the electronic activity. In some implementations, the method includes determining, by the one or more processors, a contribution score of the first entry based on a trust score of a source of the electronic activity, the contribution score used to determine a confidence score of the first value of the first field of the first node profile. In some implementations, the method includes determining the confidence score of the first value of the first field of the first node profile based on the contribution score of the first entry and respective contribution scores of one or more additional entries included in the first value data structure of the first value.

In some implementations, the method includes determining, by the one or more processors, that a second value of a second activity field-value pair matches a second value of a second node field-value pair. The method can include adding, by the one or more profiles, to a second value data structure of the second node field-value pair, a second entry identifying the electronic activity. In some implementations, the method includes determining, by the one or more processors, that a second value of a second activity field-value pair does not match a second value of a second node field-value pair. The method can include generating, by the one or more processors, a second value data structure of the node profile using the second value, the second value data structure including a second entry that identifies the electronic activity. In some implementations, the method includes identifying a first string of the plurality of strings from a portion of the electronic activity. The method can include determining a confidence score that the first string is a first name based on comparing the first string to a plurality of values of the node field-value pairs and the portion of the electronic activity from which the first string was identified. The method can include generating the match score of the electronic activity based on matching characters of the first string to one or more values node field-value pairs.

In some implementations, the method includes each node profile includes a plurality of fields, each field of the plurality of fields including one or more value data structures, each value data structure of the one or more value data structures including a value and one or more entries corresponding to respective one or more data points that support the value of the value data structure. The method can include generating the match score of the electronic activity to the first node profile includes matching the plurality of strings identified from the electronic activity to respective node field-value pairs of the first node profile.

In some implementations, the plurality of fields includes a first name field, a last name field, a phone number field, an email address field and a company name field. In some implementations, the method includes determining the subset of node profiles to correspond to a sender of the electronic activity, the subset being a first subset, the electronic activity being a first electronic activity, identifying a second electronic activity, determining the second electronic activity to be at least one of a reply to or a forward of the first electronic activity, determining a second subset of node profiles that match the second electronic activity, and updating the first subset of node profiles using the second subset of node profiles. In some implementations, the method includes increasing the match score of at least one node profile of the subset of node profiles responsive to determining that the second electronic activity is at least one of a forward or a reply to the first electronic activity. In some implementations, the method includes determining the match score based on determining that the electronic activity is at least one of a forward or a reply.

According to at least one aspect of the disclosure a method can include accessing, by one or more processors, a plurality of electronic activities transmitted or received via electronic accounts associated with a plurality of data source providers, the one or more processors maintaining a plurality of node profiles and configured to update the plurality of node profiles using the plurality of electronic activities. The method can include identifying, by the one or more processors, from data included in an electronic activity of the plurality of electronic activities to link to one or more node profiles, a plurality of strings, each string of the plurality of strings corresponding to a respective field of one or more node profiles maintained by the one or more processors. The method can include identifying, by the one or more processors, a plurality of candidate node profiles to which to link the electronic activity by comparing one or more strings of the plurality of strings to values of fields of respective candidate node profiles. The method can include generating, by the one or more processors, for each candidate node profile, a match score indicating a likelihood that the electronic activity is transmitted or received by an account corresponding to the candidate node profile based on comparing the plurality of strings included in the electronic activity to values of fields included in the candidate node profile.

The method can include determining, by the one or more processors, a subset of the plurality of candidate node profiles based on the match score of each candidate node profile of the subset satisfying a threshold. The method can include linking, by the one or more processors, the electronic activity to each candidate node profile of the subset of the plurality of candidate node profiles.

In some implementations, the method includes selecting, by the one or more processors, a first node profile of the subset of the plurality of candidate node profiles with which to match the electronic activity based on the match score of the first node profile. The method can include matching, by the one or more processors, the electronic activity with the first node profile. In some implementations, the method includes determining, by the one or more processors, a first string of the plurality of strings is a first string type that corresponds to a first field of the plurality of node profiles. The method can include determining, by the one or more processors, that the first string matches a first value of the first field of the first node profile. The method can include adding, by the one or more processors, to a first value data structure of the first value of the first field of the first node profile, a first entry identifying the electronic activity. In some implementations, the method includes determining, by the one or more processors, a contribution score of the first entry based on a trust score of a source of the electronic activity, the contribution score used to determine a confidence score of the first value of the first field of the first node profile.

In some implementations, the method includes determining the confidence score of the first value of the first field of the first node profile based on the contribution score of the first entry and respective contribution scores of one or more additional entries included in the first value data structure of the first value. In some implementations, the method includes determining, by the one or more processors, a second string of the plurality of strings is a second string type that corresponds to a second field of the plurality of node profiles. In some implementations, the method includes determining, by the one or more processors, that the second string matches a second value of the second field of the first node profile. The method can include adding, by the one or more profiles, to a second value data structure of the second value of the second field of the first node profile, a second entry identifying the electronic activity. In some implementations, the method includes determining, by the one or more processors, a second string of the plurality of strings is a second string type that corresponds to a second field of the plurality of node profiles. The method can include determining, by the one or more processors, that the second field of the first node profile does not include a value that matches the second string. The method can include generating, by the one or more processors, a second value data structure of the value that matches the second string, the second value data structure including a second entry that identifies the electronic activity.

In some implementations, the method includes identifying a first string of the plurality of strings from a portion of the electronic activity. The method can include determining a confidence score that the first string is a first name based on comparing the first string to a plurality of values of a first name field of the plurality of node profiles and the portion of the electronic activity from which the first string was identified. The method can include generating the match score of the electronic activity to the first node profile includes generating the match score based on matching the first string of characters to one or more values of the first name field of the first of node profile.

In some implementations, each node profile includes a plurality of fields, each field of the plurality of fields including one or more value data structures, each value data structure of the one or more value data structures including a value and one or more entries corresponding to respective one or more data points that support the value of the value data structure. In some implementations, the method includes generating the match score of the electronic activity to the first node profile includes matching the plurality of strings identified from the electronic activity to respective fields of the first node profile. In some implementations, the plurality of fields include a first name field, a last name field, a phone number field, an email address field and a company name field.

According to at least one aspect of the disclosure a method can include accessing, by one or more processors, a plurality of record objects of one or more systems of record, each record object of the plurality of record objects corresponding to a record object type and each comprising one or more object field-value pairs associating an object field value to a corresponding field of the record object, the systems of record corresponding to the one or more data source providers. The method can include maintaining, by the one or more processors, a plurality of node profiles corresponding to a plurality of unique entities, each node profile including one or more node field-value pairs associating a node field value to a corresponding field of the node profile. The method can include identifying, by the one or more processors, a record object to match to at least one node profile of the plurality of node profiles, each record object including a plurality of object field-value pairs associating an object field value to a corresponding field. The method can include comparing, by the one or more processors, the object field values of the one or more object field-value pairs of the record object to the corresponding node field values of the corresponding fields of the node profile. The method can include generating, by the one or more processors based on the comparison, a match score of the node profile indicating a likelihood that the record object corresponds to the node profile. The method can include determining, by the one or more processors, a subset of the plurality of node profiles with which to link the record object responsive to determining that the match score of each node profile of the subset satisfies a threshold. The method can include updating, by the one or more processors, a first value data structure of the first node field value by adding an entry identifying the record object. The method can include updating, by the one or more processors, a confidence score of the first node field value based on the entry identifying the record object.

In some implementations, the record object is a first record object and the subset is a first subset. The method can include matching, by the one or more processors, a second record object to a second subset of the plurality of node profiles. The method can include identifying, by the one or more processors, a link between the first record object and the second record object. The method can include at least one of (i) adding the second subset to the first subset or (ii) increasing a match score of comparisons of the node field-value pairs of node profiles of the second subset to the object field-value pairs of the first record object.

In some implementations, the method includes matching the record object to the first node profile by matching the record object to the first node profile based on a second object field value matching a second node field value of the first node profile, wherein the entry is a first entry, the confidence score is a first confidence score. The method can include updating, by the one or more processors, a second value data structure of the first node field value by adding a second entry identifying the record object. The method can include updating, by the one or more processors, a second confidence score of the second node field value based on the second entry identifying the record object. In some implementations, the method includes generating a contribution score of the entry identifying the record object based on a trust score assigned to the system of record associated with the record object, the contribution score contributing to the confidence score of the first node field value. In some implementations, the method includes generating the trust score of the system of record based on comparing the values of object fields of record objects of the system of record to values of node profiles having a confidence score above a predetermined threshold.

In some implementations, the contribution score is based on a time at which the record object was last updated or modified. In some implementations, matching the record object to the first node profile includes matching the record object to the first node profile based on a matching policy, the matching policy including a first rule having a first weight and a second rule having a second weight. In some implementations, the method includes maintaining, by the one or more processors, a shadow record object corresponding to the record object. The method can include adding, by the one or more processors, to the shadow record object, one or more values to one or more shadow object fields of the shadow record object determined from the first node profile to which the record object is matched. In some implementations, the method includes providing a notification to a device to update a value of the object field of the record object based on the one or more values added to the one or more shadow object fields of the shadow record object. In some implementations, the method includes determining, by the one or more processors, that a second object field value of a second object field of the record object is different from a second node field value of a corresponding field of the first node profile. The method can include determining, by the one or more processors, that the second node field value of the corresponding field has a confidence score above a predetermined threshold. The method can include generating, by the one or more processors, a request to update the second object field value to match the second node field value in the second object field of the record object responsive to determining that the second node field value of the corresponding field has a confidence score above a predetermined threshold.

According to at least one aspect of the disclosure a method can include accessing, by one or more processors, a plurality of record objects of one or more systems of record, each record object of the plurality of record objects corresponding to a record object type and comprising one or more object fields having one or more object field values, the systems of record corresponding to the one or more data source providers. The method can include maintaining, by the one or more processors, a plurality of node profiles corresponding to a plurality of unique entities, each node profile including a plurality of node profile fields, each node profile field of the plurality of node profile fields including one or more value data structures, each value data structure of the one or more value data structures including a node field value and one or more entries corresponding to respective one or more data points that support the node field value of the value data structure. The method can include identifying, by the one or more processors, a record object to match to at least one node profile of the plurality of node profiles. The method can include matching, by the one or more processors, the record object to a first node profile based on a first object field value matching a first node field value of the first node profile. The method can include updating, by the one or more processors, a first value data structure of the first node field value by adding an entry identifying the record object. The method can include updating, by the one or more processors, a confidence score of the first node field value based on the entry identifying the record object.

In some implementations, the record object is a first record object and the subset is a first subset. The method can include matching, by the one or more processors, a second record object to a second subset of the plurality of node profiles. The method can include identifying, by the one or more processors, a link between the first record object and the second record object. The method can include at least one of (i) adding the second subset to the first subset or (ii) increasing a match score of comparisons of the node field-value pairs of node profiles of the second subset to the object field-value pairs of the first record object.

In some implementations, the method includes matching the record object to the first node profile by matching the record object to the first node profile based on a second object field value matching a second node field value of the first node profile, wherein the entry is a first entry, the confidence score is a first confidence score. The method can include updating, by the one or more processors, a second value data structure of the first node field value by adding a second entry identifying the record object. The method can include updating, by the one or more processors, a second confidence score of the second node field value based on the second entry identifying the record object.

In some implementations, the method includes generating a contribution score of the entry identifying the record object based on a trust score assigned to the system of record associated with the record object, the contribution score contributing to the confidence score of the first node field value. In some implementations, the method includes generating the trust score of the system of record based on comparing the values of object fields of record objects of the system of record to values of node profiles having a confidence score above a predetermined threshold. In some implementations, the contribution score is based on a time at which the record object was last updated or modified. In some implementations, matching the record object to the first node profile includes matching the record object to the first node profile based on a matching policy, the matching policy including a first rule having a first weight and a second rule having a second weight. In some implementations, the method includes maintaining, by the one or more processors, a shadow record object corresponding to the record object. The method can include adding, by the one or more processors, to the shadow record object, one or more values to one or more shadow object fields of the shadow record object determined from the first node profile to which the record object is matched. In some implementations, the method includes providing a notification to a device to update a value of the object field of the record object based on the one or more values added to the one or more shadow object fields of the shadow record object.

In some implementations, the method includes determining, by the one or more processors, that a second object field value of a second object field of the record object is different from a second node field value of a corresponding field of the first node profile. The method can include determining, by the one or more processors, that the second node field value of the corresponding field has a confidence score above a predetermined threshold. The method can include generating, by the one or more processors, a request to update the second object field value to match the second node field value in the second object field of the record object responsive to determining that the second node field value of the corresponding field has a confidence score above a predetermined threshold. In some implementations, the method includes determining, by the one or more processors, the match score as a weighted average based on a uniqueness score of each object field value.

According to at least one aspect of the disclosure a method can include accessing, by one or more processors, at least one of i) a plurality of electronic activities transmitted or received via electronic accounts associated with one or more data source providers or ii) a plurality of record objects of one or more systems of record associated with the one or more data source providers, the one or more processors maintaining a plurality of node profiles and configured to update the plurality of node profiles using at least one of the plurality of electronic activities or the plurality of record objects. The method can include identifying, by the one or more processors, a node profile of the plurality of node profiles including a plurality of fields, each field of the plurality of fields including one or more value data structures, each value data structure of the one or more value data structures corresponding to a value and further including one or more entries, each entry of the one or more entries corresponding to respective one or more data points that include a string that matches the value of the value data structure, each data point of the one or more data points identifying a respective electronic activity of the plurality of electronic activities or a respective record object of the plurality of record objects. The method can include determining, for at least one data point of the one or more data points included in a respective value data structure of a value of a field of the plurality of fields of the node profile, a contribution score of the data point based on a time corresponding to when the data point was generated or updated. The method can include generating, by the one or more processors, a confidence score of the value of the field of the node profile based on the contribution score of the at least one data point.

In some implementations, the data point identifies an electronic activity of the plurality of electronic activities or a record object of a system of record accessible by the one or more processors. Determining the contribution score of the data point can include determining, for at least one data point of the one or more data points that includes the value of the field of the node profile, the contribution score of the at least one data point comprises determining, for each data point of the one or more data points that support the value of the field of the node profile, a respective contribution score of the data point based on a respective time corresponding to when the data point was generated or updated. Generating a confidence score of the value can include generating, by the one or more processors, the confidence score of the value of the field of the node profile based on the contribution score of the at least one data point includes generating, by the one or more processors, the confidence score of the value of the field of the node profile based on the respective contribution score of each of the one or more data points that support the value of the field of the node profile.

Determining the contribution score of the data point can include determining, for the at least one data point of the one or more data points, the contribution score of the data point includes determining, for the at least one data point, a contribution score of the data point based on a trust score assigned to a source of the data point, the trust score determined based on a type of source of the data point. The data point can be a record object. The trust score assigned to the data point can be based on a health of the system of record from which the record object was accessed. The health of the system of record from which the record object was accessed can be determined based on comparing field values of object fields included in record objects of the system of record to node profile field values of fields of one or more node profiles having respective confidence scores above a predetermined threshold.

In some implementations, the method can include receiving, by the one or more processors, a second electronic activity. The method can include determining, by the one or more processors, that the second electronic activity includes the value of the field of the node profile. The method can include generating, by the one or more processors, a second contribution score of the second electronic activity for the value of the field of the node profile. The method can include updating, by the one or more processors, the confidence score of the value based on the contribution score of the second electronic activity. In some implementations, the method can include identifying, by the one or more processors, a record object of a system of record previously not matched to the value of the field of the node profile. The method can include determining, by the one or more processors, that the record object includes the value of the field of the node profile. The method can include generating, by the one or more processors, a contribution score of the record object. The method can include updating, by the one or more processors, the confidence score of the value of the field of the node profile based on the contribution score of the record object.

In some implementations, the data point identifies an electronic activity is an automatically generated bounce back electronic activity. In some implementations, the method includes maintaining, for the value of the field of the node profile, an occurrence metric indicating a number of data points used to support the value. In some implementations, the node profile includes a first field having a first value data structure identifying a first value and the first value is assigned to the first field by linking a first electronic activity to the node profile.

In some implementations, the first value data structure includes a first entry identifying the first electronic activity and the first electronic activity is linked to the first node profile by identifying, by the one or more processors, from data included in the first electronic activity, a plurality of strings. The method can include identifying, by the one or more processors, a plurality of candidate node profiles to which to link the electronic activity by comparing one or more strings of the plurality of strings to values of fields of respective candidate node profiles. The method can include generating, by the one or more processors, for each candidate node profile, a match score indicating a likelihood that the electronic activity is transmitted or received by an account corresponding to the candidate node profile based on comparing the plurality of strings included in the electronic activity to values of fields included in the candidate node profile. The method can include linking, by the one or more processors, the first electronic activity to the first node profile based on the match score of the first node profile.

In some implementations, the value of the field of the node profile includes a first value of a first field of the node profile and the contribution score of the data point is a first contribution score of a first data point and the confidence score is a first confidence score of a first value. The method can include identifying a second value data structure of a second field of the node profile, the second value data structure corresponding to a second value of the second field and further including one or more second entries corresponding to respective one or more second data points that support the second value of the second value data structure. The method can include determining, for at least one second data point of the one or more second data points of the second value of the second field of the node profile, a second contribution score of the second data point based on a time corresponding to when the second data point was generated or updated. The method can include generating, by the one or more processors, a second confidence score of the second value of the second field of the node profile based on the second contribution score of the at least one second data point.

In some implementations, the field of the plurality of fields of the node profile comprises a second value and a corresponding second value data structure including at least one second entry identifying a second electronic activity or at least one second record object that includes a second string that matches the second value. In some implementations, the field of the plurality of fields is a first field and a second field of the plurality of fields of the node profile comprises a second value and a corresponding second value data structure including at least one second entry identifying the first electronic activity or the first record object that also includes a second string that matches the second value.

The method can include receiving, by the one or more processors, a subsequent electronic activity. The method can include linking, by the one or more processors, the electronic activity to the node profile by including one or more entries identifying the electronic activity to one or more value data structures corresponding to one or more values of one or more fields. The method can include generating, by the one or more processors, for each entry identifying the electronic activity, a contribution score of the electronic activity, the entry corresponding to a respective value data structure of a respective value of a respective field. The method can include generating, by the one or more processors, respective confidence scores for the values based on the respective contribution scores of the electronic activity. In some implementations, the electronic activity includes a signature block in the electronic activity, and linking the electronic activity to the node profile comprises extracting, by the one or more processors, from the signature block of the electronic activity, a plurality of strings. The method can include determining, by the one or more processors, using the plurality of strings extracted from the signature block, that the node profile of the plurality of node profiles includes one or more values that match respective strings of the plurality of strings.

Another aspect of the disclosure describes a system including one or more processors configured to access at least one of i) a plurality of electronic activities transmitted or received via electronic accounts associated with one or more data source providers or ii) a plurality of record objects of one or more systems of record associated with the one or more data source providers. The one or more processors can maintain a plurality of node profiles and be configured to update the plurality of node profiles using at least one of the plurality of electronic activities or the plurality of record objects. The one or more processors can identify a node profile of the plurality of node profiles including a plurality of fields. Each field of the plurality of fields includes one or more value data structures, each value data structure of the one or more value data structures corresponding to a value and further including one or more entries, each entry of the one or more entries corresponding to respective one or more data points that include a string that matches the value of the value data structure. Each data point of the one or more data points identifies a respective electronic activity of the plurality of electronic activities or a respective record object of the plurality of record objects. The system determines, for at least one data point of the one or more data points included in a respective value data structure of a value of a field of the plurality of fields of the node profile, a contribution score of the data point based on a time corresponding to when the data point was generated or updated. The system can generate a confidence score of the value of the field of the node profile based on the contribution score of the at least one data point.

In some implementations, the data point identifies an electronic activity of the plurality of electronic activities or a record object of a system of record accessible by the one or more processors. Determining the contribution score of the data point can include determining, for at least one data point of the one or more data points that includes the value of the field of the node profile, the contribution score of the at least one data point comprises determining, for each data point of the one or more data points that support the value of the field of the node profile, a respective contribution score of the data point based on a respective time corresponding to when the data point was generated or updated. Generating a confidence score of the value can include generating, by the one or more processors, the confidence score of the value of the field of the node profile based on the contribution score of the at least one data point includes generating, by the one or more processors, the confidence score of the value of the field of the node profile based on the respective contribution score of each of the one or more data points that support the value of the field of the node profile.

Determining the contribution score of the data point can include determining, for the at least one data point of the one or more data points, the contribution score of the data point includes determining, for the at least one data point, a contribution score of the data point based on a trust score assigned to a source of the data point, the trust score determined based on a type of source of the data point. The data point can be a record object. The trust score assigned to the data point can be based on a health of the system of record from which the record object was accessed. The health of the system of record from which the record object was accessed can be determined based on comparing field values of object fields included in record objects of the system of record to node profile field values of fields of one or more node profiles having respective confidence scores above a predetermined threshold.

In some implementations, the system can receive a second electronic activity. The system can determine that the second electronic activity includes the value of the field of the node profile. The system can generate a second contribution score of the second electronic activity for the value of the field of the node profile. The system can update the confidence score of the value based on the contribution score of the second electronic activity. In some implementations, the system can identify a record object of a system of record previously not matched to the value of the field of the node profile. The system can determine that the record object includes the value of the field of the node profile. The method can include generating, by the one or more processors, a contribution score of the record object. The method can include updating, by the one or more processors, the confidence score of the value of the field of the node profile based on the contribution score of the record object.

In some implementations, the data point identifies an electronic activity is an automatically generated bounce back electronic activity. In some implementations, the system can maintain, for the value of the field of the node profile, an occurrence metric indicating a number of data points used to support the value. In some implementations, the node profile includes a first field having a first value data structure identifying a first value and the first value is assigned to the first field by linking a first electronic activity to the node profile.

In some implementations, the first value data structure includes a first entry identifying the first electronic activity and the first electronic activity is linked to the first node profile by identifying, by the one or more processors, from data included in the first electronic activity, a plurality of strings. The method can include identifying, by the one or more processors, a plurality of candidate node profiles to which to link the electronic activity by comparing one or more strings of the plurality of strings to values of fields of respective candidate node profiles. The method can include generating, by the one or more processors, for each candidate node profile, a match score indicating a likelihood that the electronic activity is transmitted or received by an account corresponding to the candidate node profile based on comparing the plurality of strings included in the electronic activity to values of fields included in the candidate node profile. The method can include linking, by the one or more processors, the first electronic activity to the first node profile based on the match score of the first node profile.

In some implementations, the value of the field of the node profile includes a first value of a first field of the node profile and the contribution score of the data point is a first contribution score of a first data point and the confidence score is a first confidence score of a first value. The system can identify a second value data structure of a second field of the node profile, the second value data structure corresponding to a second value of the second field and further including one or more second entries corresponding to respective one or more second data points that support the second value of the second value data structure. The system can determine, for at least one second data point of the one or more second data points of the second value of the second field of the node profile, a second contribution score of the second data point based on a time corresponding to when the second data point was generated or updated. The system can generate a second confidence score of the second value of the second field of the node profile based on the second contribution score of the at least one second data point.

In some implementations, the field of the plurality of fields of the node profile comprises a second value and a corresponding second value data structure including at least one second entry identifying a second electronic activity or at least one second record object that includes a second string that matches the second value. In some implementations, the field of the plurality of fields is a first field and a second field of the plurality of fields of the node profile comprises a second value and a corresponding second value data structure including at least one second entry identifying the first electronic activity or the first record object that also includes a second string that matches the second value.

The system can be configured to a subsequent electronic activity. The system can be configured to link the electronic activity to the node profile by including one or more entries identifying the electronic activity to one or more value data structures corresponding to one or more values of one or more fields. The system can generate, for each entry identifying the electronic activity, a contribution score of the electronic activity, the entry corresponding to a respective value data structure of a respective value of a respective field. The system can generate respective confidence scores for the values based on the respective contribution scores of the electronic activity. In some implementations, the electronic activity includes a signature block in the electronic activity, and linking the electronic activity to the node profile comprises extracting, by the one or more processors, from the signature block of the electronic activity, a plurality of strings. The system can determine, using the plurality of strings extracted from the signature block, that the node profile of the plurality of node profiles includes one or more values that match respective strings of the plurality of strings.

One aspect of the present disclosure relates to a method for detecting events based on updates to node profiles from electronic activities. The method may include accessing, by one or more processors, an electronic activity transmitted or received via an electronic account associated with a data source provider. The method may include generating, by the one or more processors, by parsing the electronic activity, a plurality of activity field-value pairs from an electronic activity of the plurality of electronic activities. Each activity field-value pair may include an activity value associated with an activity field. The method may include maintaining, by the one or more processors, in one or more data structures stored in a memory coupled to the one or more processors, a plurality of node profiles. Each node profile of the plurality of node profiles may include one or more node field-value pairs. Each node field-value pair may include a node value associated with a node field. Each node value may be associated with a confidence score. The method may include identifying, by the one or more processors, a first state of a first node profile of the plurality of node profiles. The first state may correspond to a first node field-value pair of the one or more node field-value pairs of the first node profile. The method may include updating, by the one or more processors, the first node profile using the electronic activity. The method may include identifying, by the one or more processors, a second state of the first node profile subsequent to updating the first node profile using the electronic activity. The method may include detecting, by the one or more processors, a state change of the first node profile based on the first state and the second state. The state change may include at least one of a tag assigned to the first node profile or an update regarding one or more node values associated with a first node field of the first node field-value pair. The method may include determining, by the one or more processors, that the state change satisfies an event condition. The event condition corresponding to one or more may be predetermined node fields of the one or more node field-value pairs of the first node profile. The method may include storing, by the one or more processors, responsive to the state change satisfying the event condition, an association between the first node profile and an event type corresponding to the event condition.

In some implementations of the method, it may include selecting, by the one or more processors, the first node profile to update using the electronic activity based on a match score of the first node profile indicating a likelihood that the electronic activity is transmitted or received by the electronic account corresponding to the first node profile or a server corresponding to the electronic account.

In some implementations of the method, determining that the state change may satisfy the event condition includes determining that the first node field is at least one of a job title field, a company field, or a last name field. In some implementations of the method, the update may indicate that the one or more node values include a new value associated with the first field in the second state and not in the first state.

In some implementations of the method, determining that the state change may satisfy the event condition includes determining that the first node field includes at least two of a city field, a state field, or a phone number field. In some implementations of the method, the update may indicate the one or more node values to include a new value associated with the first field in the second state and not in the first state.

In some implementations of the method, updating the first node profile may include updating a second field-value pair corresponding to the first field using the electronic activity. In some implementations of the method, it may include identifying, by the one or more processors, a first field-value pair corresponding to the first field having a first highest confidence score in the first state. In some implementations of the method, it may include identifying, by the one or more processors, that a confidence score of the second field-value pair is greater than a corresponding confidence score of the first field-value pair in the second state. In some implementations of the method, determining that the state change may satisfy the event condition includes determining that the confidence score of the second field-value pair is greater than the corresponding confidence score of the first field-value pair in the second state.

In some implementations of the method, it may include receiving, by the one or more processors, a second electronic activity. In some implementations of the method, it may include identifying, by the one or more processors, the first node profile and at least one candidate node profile of the plurality of node profiles to which to link the second electronic activity. In some implementations of the method, it may include selecting, by the one or more processors, the first node profile to which to link the second electronic activity responsive to detecting the state change to include the tag assigned to the first node profile.

In some implementations of the method, it may include updating, by the one or more processors, responsive to the state change satisfying the event condition, a hierarchy data structure that includes the first node profile.

In some implementations of the method, the tag may be indicative of the electronic activity being a bounce-back electronic activity.

In some implementations of the method, it may include determining, by the one or more processors, from the electronic activity, at least one of a time or date at which the first electronic activity was transmitted or received. In some implementations of the method, storing, by the one or more processors, the association between the first node profile and an event type corresponding to the event condition may include storing the association between the first node profile and the event type corresponding to the event condition and the at least one of the time or the date.

Another aspect of the present disclosure relates to a system. The system may include one or more hardware processors configured by machine-readable instructions. The processor(s) may be configured to access, by one or more processors, an electronic activity transmitted or received via an electronic account associated with a data source provider. The processor(s) may be configured to generate, by the one or more processors, by parsing the electronic activity, a plurality of activity field-value pairs from an electronic activity of the plurality of electronic activities. Each activity field-value pair may include an activity value associated with an activity field. The processor(s) may be configured to maintain, by the one or more processors, in one or more data structures stored in a memory coupled to the one or more processors, a plurality of node profiles. Each node profile of the plurality of node profiles may include one or more node field-value pairs. Each node field-value pair may include a node value associated with a node field. Each node value may be associated with a confidence score. The processor(s) may be configured to identify, by the one or more processors, a first state of a first node profile of the plurality of node profiles. The first state may correspond to a first node field-value pair of the one or more node field-value pairs of the first node profile. The processor(s) may be configured to update, by the one or more processors, the first node profile using the electronic activity. The processor(s) may be configured to identify, by the one or more processors, a second state of the first node profile subsequent to updating the first node profile using the electronic activity. The processor(s) may be configured to detect, by the one or more processors, a state change of the first node profile based on the first state and the second state. The state change may include at least one of a tag assigned to the first node profile or an update regarding one or more node values associated with a first node field of the first node field-value pair. The processor(s) may be configured to determine, by the one or more processors, that the state change satisfies an event condition. The event condition corresponding to one or more may be predetermined node fields of the one or more node field-value pairs of the first node profile. The processor(s) may be configured to store, by the one or more processors, responsive to the state change satisfying the event condition, an association between the first node profile and an event type corresponding to the event condition.

In some implementations of the system, the processor(s) may be configured to select, by the one or more processors, the first node profile to update using the electronic activity based on a match score of the first node profile indicating a likelihood that the electronic activity is transmitted or received by the electronic account corresponding to the first node profile or a server corresponding to the electronic account.

In some implementations of the system, determining that the state change may satisfy the event condition includes determining that the first node field is at least one of a job title field, a company field, or a last name field. In some implementations of the system, the update may indicate that the one or more node values include a new value associated with the first field in the second state and not in the first state.

In some implementations of the system, determining that the state change may satisfy the event condition includes determining that the first node field includes at least two of a city field, a state field, or a phone number field. In some implementations of the system, the update may indicate the one or more node values to include a new value associated with the first field in the second state and not in the first state.

In some implementations of the system, updating the first node profile may include updating a second field-value pair corresponding to the first field using the electronic activity. In some implementations of the system, the processor(s) may be configured to identify, by the one or more processors, a first field-value pair corresponding to the first field having a first highest confidence score in the first state. In some implementations of the system, the processor(s) may be configured to identify, by the one or more processors, that a confidence score of the second field-value pair is greater than a corresponding confidence score of the first field-value pair in the second state. In some implementations of the system, determining that the state change may satisfy the event condition includes determining that the confidence score of the second field-value pair is greater than the corresponding confidence score of the first field-value pair in the second state.

In some implementations of the system, the processor(s) may be configured to receive, by the one or more processors, a second electronic activity. In some implementations of the system, the processor(s) may be configured to identify, by the one or more processors, the first node profile and at least one candidate node profile of the plurality of node profiles to which to link the second electronic activity. In some implementations of the system, the processor(s) may be configured to select, by the one or more processors, the first node profile to which to link the second electronic activity responsive to detecting the state change to include the tag assigned to the first node profile.

In some implementations of the system, the processor(s) may be configured to update, by the one or more processors, responsive to the state change satisfying the event condition, a hierarchy data structure that includes the first node profile.

In some implementations of the system, the tag may be indicative of the electronic activity being a bounce-back electronic activity.

In some implementations of the system, the processor(s) may be configured to determine, by the one or more processors, from the electronic activity, at least one of a time or date at which the first electronic activity was transmitted or received. In some implementations of the system, storing, by the one or more processors, the association between the first node profile and an event type corresponding to the event condition may include storing the association between the first node profile and the event type corresponding to the event condition and the at least one of the time or the date.

Yet another aspect of the present disclosure relates to a non-transitory computer-readable storage medium having instructions embodied thereon, the instructions being executable by one or more processors to perform a method. The method may include accessing, by one or more processors, an electronic activity transmitted or received via an electronic account associated with a data source provider. The method may include generating, by the one or more processors, by parsing the electronic activity, a plurality of activity field-value pairs from an electronic activity of the plurality of electronic activities. Each activity field-value pair may include an activity value associated with an activity field. The method may include maintaining, by the one or more processors, in one or more data structures stored in a memory coupled to the one or more processors, a plurality of node profiles. Each node profile of the plurality of node profiles may include one or more node field-value pairs. Each node field-value pair may include a node value associated with a node field. Each node value may be associated with a confidence score. The method may include identifying, by the one or more processors, a first state of a first node profile of the plurality of node profiles. The first state may correspond to a first node field-value pair of the one or more node field-value pairs of the first node profile. The method may include updating, by the one or more processors, the first node profile using the electronic activity. The method may include identifying, by the one or more processors, a second state of the first node profile subsequent to updating the first node profile using the electronic activity. The method may include detecting, by the one or more processors, a state change of the first node profile based on the first state and the second state. The state change may include at least one of a tag assigned to the first node profile or an update regarding one or more node values associated with a first node field of the first node field-value pair. The method may include determining, by the one or more processors, that the state change satisfies an event condition. The event condition corresponding to one or more may be predetermined node fields of the one or more node field-value pairs of the first node profile. The method may include storing, by the one or more processors, responsive to the state change satisfying the event condition, an association between the first node profile and an event type corresponding to the event condition.

One aspect of the present disclosure relates to a method. The method includes accessing, by one or more processors, a plurality of electronic activities transmitted or received via a plurality of electronic accounts associated with a plurality of data source providers. The method includes identifying, by the one or more processors for a node profile maintained by the one or more processors, a set of electronic activities sent from or received by each electronic account linked to the node profile within a time period. The method includes identifying, by the one or more processors, for each electronic activity of the set of electronic activities, a timestamp at which the electronic activity was sent or received. The method includes generating, by the one or more processors, for each time interval within the time period, a temporal distribution of electronic activity for the time interval based on respective timestamps of each electronic activity of the set of electronic activities within the time interval. The method includes determining, by the one or more processors, an electronic activity pattern based on the temporal distribution of electronic activity for each time interval within the time period. The method includes identifying, by the one or more processors, based on the electronic activity pattern, a first region of the electronic activity pattern satisfying a region identification policy.

The method includes storing, by the one or more processors in one or more data structures, an association between the first region and the node profile.

Another aspect of the present disclosure relates to a system. The system includes one or more hardware processors configured by machine-readable instructions to access a plurality of electronic activities transmitted or received via a plurality of electronic accounts associated with a plurality of data source providers. The hardware processors are further configured to identify, by the one or more processors for a node profile maintained by the one or more processors, a set of electronic activities sent from or received by each electronic account linked to the node profile within a time period. The hardware processors are further configured to identify for each electronic activity of the set of electronic activities, a timestamp at which the electronic activity was sent or received. The hardware processors are further configured to generate for each time interval within the time period, a temporal distribution of electronic activity for the time interval based on respective timestamps of each electronic activity of the set of electronic activities within the time interval. The hardware processors are further configured to determine an electronic activity pattern based on the temporal distribution of electronic activity for each time interval within the time period. The hardware processors are further configured to identify, based on the electronic activity pattern, a first region of the electronic activity pattern satisfying a region identification policy. The hardware processors are further configured to store, in one or more data structures, an association between the first region and the node profile.

Another aspect of the present disclosure relates to a non-transitory computer-readable storage medium having instructions embodied thereon. The instructions are executable by one or more processors to perform a method. The method includes accessing, by one or more processors, a plurality of electronic activities transmitted or received via a plurality of electronic accounts associated with a plurality of data source providers. The method includes identifying, by the one or more processors for a node profile maintained by the one or more processors, a set of electronic activities sent from or received by each electronic account linked to the node profile within a time period. The method includes identifying, by the one or more processors, for each electronic activity of the set of electronic activities, a timestamp at which the electronic activity was sent or received. The method includes generating, by the one or more processors, for each time interval within the time period, a temporal distribution of electronic activity for the time interval based on respective timestamps of each electronic activity of the set of electronic activities within the time interval. The method includes determining, by the one or more processors, an electronic activity pattern based on the temporal distribution of electronic activity for each time interval within the time period. The method includes identifying, by the one or more processors, based on the electronic activity pattern, a first region of the electronic activity pattern satisfying a region identification policy. The method includes storing, by the one or more processors in one or more data structures, an association between the first region and the node profile.

According to at least one aspect of the disclosure a method can include maintaining, by the one or more processors, a plurality of node profiles including one or more field-value pairs. At least one of the field-value pairs of each node profile identifies a phone number corresponding to the node profile. The method further includes generating, by the one or more processors, a plurality of activity field-value pairs from an electronic activity ingested by the one or more processors. Each activity field-value pair of the plurality of activity field-value pairs corresponds to at least one participant of the electronic activity. The method further includes parsing, by the one or more processors, the electronic activity to identify a string including at least a predetermined number of numerical characters satisfying one or more predetermined regular expression (regex) patterns corresponding to an electronic activity phone number and determining, by the one or more processors, a type of phone number to which the electronic activity phone number corresponds using a data structure including a plurality of phone numbers. Each phone number of the plurality of phone numbers are stored in the data structure with a corresponding field type identifying a phone number type of the phone number. The phone number types are determined based on matching at least a portion of the electronic activity phone number to a corresponding portion of each of the plurality of phone numbers. The method further includes identifying, by the one or more processors, a node profile of the plurality of node profiles corresponding to the sender or one or more recipients of the electronic activity to which the electronic activity phone number corresponds and updating, by the one or more processors, the identified node profile by adding the electronic activity phone number as a new value of a field corresponding to the determined phone number type or updating a confidence score of an existing field-value pair corresponding to the electronic activity phone number.

In some embodiments, the method further includes determining, by the one or more processors, from content included in the electronic activity, that the phone number type of the electronic activity phone number cannot be determined based on the content included in the electronic activity.

In some embodiments, the data structure includes a count for each phone number of the plurality of phone numbers corresponding to a number of the node profiles that include a field-value pair having a value that matches the phone number.

In some embodiments, the phone number type is one of a home phone number, a cell phone number, a group entity general number, or a group entity direct number.

In some embodiments, the method further includes accessing, by the one or more processors, a plurality of electronic activities and generating, by the one or more processors, the node profiles based on information included in the plurality of electronic activities.

In some embodiments, the method further includes generating, by the one or more processors, the data structure to include, for each phone number included in the data structure, an aggregate confidence score based on respective individual confidence scores of values of field-value pairs of node profiles that match the phone number.

In some embodiments, the method further includes generating, by the one or more processors, the data structure to include, for each phone number included in the data structure, a frequency of the phone number within the data structure.

In some embodiments, the method further includes generating, by the one or more processors, the data structure to include, for a first set of phone numbers included in the data structure, a value corresponding to a group entity name.

In some embodiments, the plurality of node profiles includes a plurality of member node profiles. The method may further include accessing, by the one or more processors, a publicly available database to determine a phone number of a group entity associated with a group node profile that is linked to the plurality of node profiles, comparing the electronic activity phone number to the phone number of the group entity to determine a match, and determining, by the one or more processors, that the phone number type of the electronic activity phone number is a group entity phone number based on the match.

In some embodiments, the data structure includes, for each phone number included in the data structure, a frequency of the phone number within the data structure or a phone number type of the phone number. The method may further include determining the type of phone number using the probabilistic model includes determining the probability score based on the frequency of the phone number.

In some embodiments, the method further includes determining, by the one or more processors, a first field value identifying a residential address of the participant, determining, by the one or more processors, a prefix of the electronic activity phone number, determining, by the one or more processors, a location corresponding to the prefix of the electronic activity phone number, and classifying, by the one or more processors, the electronic activity phone number as a personal phone number responsive to determining that the location corresponding to the prefix matches a portion of the physical or mailing address.

In some embodiments, the method further includes determining, by the one or more processors, a first phone number prefix corresponding to a residential address of the at least one participant, determining, by the one or more processors, a second phone number prefix of the electronic activity phone number, determining, by the one or more processors, that second prefix doesn't match the first prefix, and classifying, by the one or more processors, the electronic activity phone number as a cell phone number responsive to determining that the second phone number prefix of the electronic activity phone number does not match the first phone number prefix corresponding to a residential address of the at least one participant.

In some embodiments, the method further includes parsing, by the one or more processors, the electronic activity to identify an extension number associated with the electronic activity phone number and updating, by the one or more processors, the identified node profile by adding the electronic activity phone number and the extension number as the new value of the field corresponding to the determined phone number type.

According to at least another aspect of the disclosure a system can include one or more hardware processors configured by machine-readable instructions to maintain a plurality of node profiles including one or more field-value pairs. At least one of the field-value pairs of each node profile identifies a phone number corresponding to the node profile. The one or more hardware processors are further configured by machine-readable instructions to generate a plurality of activity field-value pairs from an electronic activity ingested by the one or more hardware processors. Each activity field-value pair of the plurality of activity field-value pairs corresponds to at least one participant of the electronic activity. The one or more hardware processors are further configured by machine-readable instructions to parse the electronic activity to identify a string including at least a predetermined number of numerical characters satisfying one or more predetermined regular expression (regex) patterns corresponding to an electronic activity phone number and determine a type of phone number to which the electronic activity phone number corresponds using a data structure including a plurality of phone numbers. Each phone number of the plurality of phone numbers is stored in the data structure with a corresponding field type identifying a phone number type of the phone number. The phone number type is determined based on matching at least a portion of the electronic activity phone number to a corresponding portion of each of the plurality of phone numbers.

The one or more hardware processors are further configured by machine-readable instructions to identify a node profile of the plurality of node profiles corresponding to the sender or one or more recipients of the electronic activity to which the electronic activity phone number corresponds and update the identified node profile by adding the electronic activity phone number as a new value of a field corresponding to the determined phone number type or updating a confidence score of an existing field-value pair corresponding to the electronic activity phone number.

In some embodiments, the one or more hardware processors are further configured by machine-readable instructions to determine from content included in the electronic activity, that the phone number type of the electronic activity phone number cannot be determined based on the content included in the electronic activity.

In some embodiments, the data structure includes a count for each phone number of the plurality of phone numbers corresponding to a number of the node profiles that include a field-value pair having a value that matches the phone number.

In some embodiments, the phone number type is one of a home phone number, a cell phone number, a group entity general number, or a group entity direct number.

In some embodiments, the one or more hardware processors are further configured by machine-readable instructions to access a plurality of electronic activities and generate the node profiles based on information included in the plurality of electronic activities.

According to at least one aspect of the disclosure a non-transitory computer-readable storage medium having instructions embodied thereon, the instructions being executable by one or more processors to maintain a plurality of node profiles including one or more field-value pairs. At least one of the field-value pairs of each node profile identifies a phone number corresponding to the node profile. The one or more hardware processors are further configured by machine-readable instructions to generate a plurality of activity field-value pairs from an electronic activity ingested by the one or more hardware processors. Each activity field-value pair of the plurality of activity field-value pairs corresponds to at least one participant of the electronic activity. The one or more hardware processors are further configured by machine-readable instructions to parse the electronic activity to identify a string including at least a predetermined number of numerical characters satisfying one or more predetermined regular expression (regex) patterns corresponding to an electronic activity phone number and determine a type of phone number to which the electronic activity phone number corresponds using a data structure including a plurality of phone numbers. Each phone number of the plurality of phone numbers is stored in the data structure with a corresponding field type identifying a phone number type of the phone number. The phone number type is determined based on matching at least a portion of the electronic activity phone number to a corresponding portion of each of the plurality of phone numbers. The one or more hardware processors are further configured by machine-readable instructions to identify a node profile of the plurality of node profiles corresponding to the sender or one or more recipients of the electronic activity to which the electronic activity phone number corresponds and update the identified node profile by adding the electronic activity phone number as a new value of a field corresponding to the determined phone number type or updating a confidence score of an existing field-value pair corresponding to the electronic activity phone number.

The present disclosure relates to systems and methods for confirming a scheduled event based on one or more electronic activities. The present technical solution dynamically identifies one or more electronic activities associated with an electronic activity corresponding to the scheduled event. In response to identifying the one or more electronic activities, the present technical solution can assign one or more tags indicating a status of the scheduled event and continually update the one or more tags.

One aspect of the present disclosure relates to a method for confirming a scheduled event based on one or more electronic activities. The method may include identifying, by one or more processors, an electronic activity corresponding to a scheduled event and identifying a plurality of candidate participants. The method may include accessing, by the one or more processors, for a first participant of the candidate participants, a plurality of electronic activities corresponding to an electronic account of the first participant. The method may include extracting, by the one or more processors, for each electronic activity of the plurality of electronic activities, a feature set based on participants included in the electronic activity or content included in a body of the electronic activity. The feature set can include one or more features. The method may include determining, by the one or more processors, for each electronic activity of the plurality of electronic activities, a relevancy score between the electronic activity and the scheduled event based on the extracted feature set. The method may include identifying, by the one or more processors, a subset of electronic activities of the plurality of electronic activities that have respective relevancy scores that satisfy a first threshold value. The method may include determining, by the one or more processors, a probability score indicating a likelihood that the first participant attended the scheduled event based on the subset of electronic activities. The method may include generating, by the one or more processors responsive to the probability score satisfying a second threshold value, an attendance tag indicating that the first participant attended the scheduled event. The method may include storing, by the one or more processors, in one or more data structures, an association between the attendance tag and the electronic activity corresponding to the scheduled event.

In some embodiments, the method may further include determining, by the one or more processors, a confidence score for the attendance tag based on the probability score. The method may further include assigning, by the one or more processors, the confidence score to the attendance tag.

In some embodiments, the method may further include accessing, by the one or more processors, a second electronic activity. The method may further include extracting, by the one or more processors, for the second electronic activity, a second feature set including one or more features based on participants included in the second electronic activity or content included in a body of the second electronic activity. The method may further include determining, by the one or more processors, based on the second feature set of the second electronic activity, that a relevancy score between the second electronic activity and the scheduled event satisfies the first threshold value. The method may further include updating, by the one or more processors, the confidence score of the attendance tag based on the second electronic activity.

In some embodiments, the probability score indicating the likelihood that the first participant attended the scheduled event is a first probability score. The method may further include determining, by the one or more processors, a second probability score indicating a likelihood that the scheduled event occurred, based on the subset of electronic activities. The method may further include generating, by the one or more processors, responsive to determining that the second probability score exceeds a predetermined event occurrence threshold, an occurrence tag indicating that the scheduled event occurred. The method may further include storing, by the one or more processors, an association between the occurrence tag and the electronic activity corresponding to the scheduled event.

In some embodiments the method may further include determining, by the one or more processors, a confidence score for the occurrence tag based on the second probability score. The method may further include assigning, by the one or more processors, the confidence score to the occurrence tag.

In some embodiments, the method may further include parsing, by the one or more processors, the subset of electronic activities to identify a number of the candidate participants that attended the scheduled event. The method may further include updating, by the one or more processors, the confidence score of the occurrence tag based on the number of the candidate participants that attended the scheduled event.

In some embodiments, the method may further include determining, by the one or more processors, an attendance rate of the scheduled event, based on the number of the candidate participants that attended the scheduled event. The method may further include generating, by the one or more processors, an attendance metric for the scheduled event. The method may further include transmitting, by the one or more processors, data using the attendance metric to a computing device of an organizer of the scheduled event.

In some embodiments, the method may further include accessing, by the one or more processors, a performance profile of a node profile corresponding to the first participant. The method may further include updating, by the one or more processors, the performance profile of the first participant responsive to the attendance tag.

In some embodiments, the method may further include parsing, by the one or more processors, the subset of electronic activities to identify one of an electronic access values associated with the scheduled event or a physical address associated with the scheduled event. The method may further include determining, by the one or more processors, an event type of the scheduled event based on identifying the electronic access value or the physical address associated with the scheduled event.

In some embodiments, the method may further include determining, by the one or more processors, that the scheduled event corresponds to a first event type responsive to determining that the electronic activity associated with the scheduled event satisfies a first event type detection policy.

In some embodiments, the method may further include identifying, by the one or more processors, a first electronic activity of the subset of electronic activities of the first participant having a recipient identifier that matches a communication string included in the electronic activity corresponding to the scheduled event. The method may further include generating, by the one or more processors, the attendance tag indicating that the first participant attended the scheduled event responsive to identifying the first electronic activity of the one or more electronic activities having the recipient identifier that matches the communication string included in the electronic activity corresponding to the scheduled event.

In some embodiments, the first electronic activity includes an electronic voice call. The method may further include determining, by the one or more processors, a duration of the electronic voice call. The method may further include generating the attendance tag indicating that the first participant attended the scheduled event responsive to determining that the duration of the electronic voice call satisfies a duration threshold.

In some embodiments, the method may further include parsing, by the one or more processors, at least one electronic activity of the subset sent or received within a predetermined time period of the scheduled event to determine a location associated with the at least one electronic activity. The method may further include comparing the location to a physical address included in the electronic activity corresponding to the scheduled event to determine a proximity between the location and the physical address. The method may further include generating the attendance tag indicating that the first participant attended the scheduled event responsive to determining that the proximity is within a predetermined proximity threshold.

In some embodiments, parsing the at least one electronic activity of the subset of electronic activities may include extracting, by the one or more processors, from the at least one electronic activity, at least one of an internet protocol (IP) address of a computing device of the first participant or a GPS location of the first participant within the predetermined time period.

In some embodiments, the attendance tag includes a first attendance tag. The method may further include accessing, by the one or more processors, for a second participant of the candidate participants, a second plurality of electronic activities corresponding to a second electronic account of the second participant. The method may further include extracting, by the one or more processors, for each electronic activity of the second plurality of electronic activities, a second feature set including one or more features based on participants included in the electronic activity or content included in a body of the electronic activity. The method may further include determining, by the one or more processors, for each electronic activity of the second plurality of electronic activities, a second relevancy score between the electronic activity and the scheduled event based on the extracted feature set. The method may further include identifying, by the one or more processors, a second subset of electronic activities of the second plurality of electronic activities that have respective relevancy scores that satisfy a second threshold value. The method may further include determining, by the one or more processors, a second probability score indicating a likelihood that the second participant attended the scheduled event based on the second subset of electronic activities. The method may further include generating, by the one or more processors responsive to the probability score not satisfying the second threshold value, a second attendance tag indicating that the second participant did not attend the scheduled event. The method may further include storing, by the one or more processors, in the one or more data structures, an association between the second attendance tag and the electronic activity corresponding to the scheduled event.

In some embodiments, the method may further include identifying, by the one or more processors, for each electronic activity of the plurality of electronic activities, a context of the electronic activity. As such, determining the relevancy score between the electronic activity and the scheduled event may further include determining the relevancy score based on the context.

In some embodiments, the first electronic activity may include an electronic voice call. The method may further include receiving, by the one or more processors from a data source provider corresponding to a host of the scheduled event, at least one phone number that participated in the electronic voice call. The method may further include matching, by the one or more processors, the at least one phone number that participated in the electronic voice call with a phone number of the first participant. As such, determining the probability score indicating the likelihood that the first participant attended the scheduled event may further include determining the probability score based on the match.

In some embodiments, the first electronic activity may include an electronic voice call. The method may further include accessing, by the one or more processors, audio information corresponding to the electronic voice call. The method may further include identifying, by the one or more processors, a first acoustic fingerprint of an attendee of the electronic voice call, based on the audio information. The method may further include matching, by the one or more processors, the first acoustic fingerprint of the attendee with a second acoustic fingerprint of the first participant. As such, determining the probability score indicating the likelihood that the first participant attended the scheduled event may further include determining the probability score based on the match.

In some embodiments, the method may further include receiving, by the one or more processors from a data source provider, travel information for the first participant, the travel information corresponding to travel within a predetermined time period of the scheduled event. The method may further include extracting, by the one or more processors, a location from the travel information. The method may further include comparing the location to a physical address included in the electronic activity corresponding to the scheduled event to determine a proximity between the location and the physical address. As such, generating the attendance tag may further include generating, by the one or more processors, the attendance tag indicating that the first participant attended the scheduled event responsive to determining that the proximity is within a predetermined proximity threshold.

In some embodiments, the method may further include determining, by the one or more processors, a second subset of the plurality of electronic activities that were transmitted by an electronic account of the first participant during the scheduled event. The method may further include generating, by the one or more processors, a meeting engagement score based on a number of electronic activities included in the second subset.

In some embodiments, the method may further include determining, by the one or more processors from the electronic activities, a percentage of the second subset of electronic activities that are relevant to the event. The method may further include adjusting, by the one or more processors, the engagement score based on the percentage.

Another aspect of the present disclosure relates to a system configured to confirm a scheduled event based on one or more electronic activities. The system may include one or more hardware processors configured by machine-readable instructions. The processor(s) may be configured to identify an electronic activity corresponding to a scheduled event and identifying a plurality of candidate participants. The processor(s) may be configured to access, for a first participant of the candidate participants, a plurality of electronic activities corresponding to an electronic account of the first participant. The processor(s) may be configured to extract, for each electronic activity of the plurality of electronic activities, a feature set including one or more features based on participants included in the electronic activity or content included in a body of the electronic activity. The processor(s) may be configured to determine, for each electronic activity of the plurality of electronic activities, a relevancy score between the electronic activity and the scheduled event based on the extracted feature set. The processor(s) may be configured to identify a subset of electronic activities of the plurality of electronic activities that have respective relevancy scores that satisfy a first threshold value. The processor(s) may be configured to determine a probability score indicating a likelihood that the first participant attended the scheduled event based on the subset of electronic activities. The processor(s) may be configured to generate, responsive to the probability score satisfying a second threshold value, an attendance tag indicating that the first participant attended the scheduled event. The processor(s) may be configured to store, in one or more data structures, an association between the attendance tag and the electronic activity corresponding to the scheduled event.

In some embodiments, the processor(s) may be further configured to determine a confidence score for the attendance tag based on the probability score. The processor(s) may be further configured to assign the confidence score to the attendance tag.

In some embodiments, the processor(s) may be further configured to access a second electronic activity. The processor(s) may be further configured to extract, for the second electronic activity, a second feature set including one or more features based on participants included in the second electronic activity or content included in a body of the second electronic activity. The processor(s) may be further configured to determine, based on the second feature set of the second electronic activity, that a relevancy score between the second electronic activity and the scheduled event satisfies the first threshold value. The processor(s) may be further configured to update the confidence score of the attendance tag based on the second electronic activity.

In some embodiments, the probability score indicating the likelihood that the first participant attended the scheduled event is a first probability score. The processor(s) may be further configured to determine a second probability score indicating a likelihood that the scheduled event occurred, based on the subset of electronic activities. The processor(s) may be further configured to generate, responsive to determining that the second probability score exceeds a predetermined event occurrence threshold, an occurrence tag indicating that the scheduled event occurred. The processor(s) may be further configured to store an association between the occurrence tag and the electronic activity corresponding to the scheduled event.

Yet another aspect of the present disclosure relates to a non-transitory computer-readable storage medium having instructions embodied thereon, the instructions being executable by one or more processors to perform a method for confirming a scheduled event based on one or more electronic activities. The method may include identifying, by one or more processors, an electronic activity corresponding to a scheduled event and identifying a plurality of candidate participants. The method may include accessing, by the one or more processors, for a first participant of the candidate participants, a plurality of electronic activities corresponding to an electronic account of the first participant. The method may include extracting, by the one or more processors, for each electronic activity of the plurality of electronic activities, a feature set based on participants included in the electronic activity or content included in a body of the electronic activity. The feature set can include one or more features. The method may include determining, by the one or more processors, for each electronic activity of the plurality of electronic activities, a relevancy score between the electronic activity and the scheduled event based on the extracted feature set. The method may include identifying, by the one or more processors, a subset of electronic activities of the plurality of electronic activities that have respective relevancy scores that satisfy a first threshold value. The method may include determining, by the one or more processors, a probability score indicating a likelihood that the first participant attended the scheduled event based on the subset of electronic activities. The method may include generating, by the one or more processors responsive to the probability score satisfying a second threshold value, an attendance tag indicating that the first participant attended the scheduled event. The method may include storing, by the one or more processors, in one or more data structures, an association between the attendance tag and the electronic activity corresponding to the scheduled event.

One aspect of the present disclosure relates to a system for estimating time to perform, generate or otherwise spend on electronic activities. The system may include one or more hardware processors configured by machine-readable instructions. The processor(s) may be configured to identify, by one or more processors, an electronic activity identifying a sender and one or more recipients and a body including content. The processor(s) may be configured to determine, by the one or more processors, using a quality estimation model, a quality of electronic activity score corresponding to an estimated quality of the electronic activity. The determining may further include.

The processor(s) may be configured to pars, by the one or more processors, the content of the electronic activity. The processor(s) may be configured to generate, by the one or more processors, using a language complexity determination engine, a language complexity score indicating a level of language complexity. The processor(s) may be configured to determine, by the one or more processors, a character count or word count. The processor(s) may be configured to determine, by the one or more processors, an estimated amount of time taken to generate the electronic activity using the language complexity score and the character count or word count. The processor(s) may be configured to generate, by the one or more processors, the QoE score corresponding to the estimated quality based on the estimated amount of time. The processor(s) may be configured to store, by the one or more processors, in a data structure, an association between the electronic activity and the QoE score.

Another aspect of the present disclosure relates to a method. The method may include identifying, by one or more processors, an electronic activity identifying a sender and one or more recipients and a body including content. The method may include determining, by the one or more processors, using a quality estimation model, a quality of electronic activity score corresponding to an estimated quality of the electronic activity. The determining may further include. The method may include parsing, by the one or more processors, the content of the electronic activity. The method may include generating, by the one or more processors, using a language complexity determination engine, a language complexity score indicating a level of language complexity. The method may include determining, by the one or more processors, a character count or word count. The method may include determining, by the one or more processors, an estimated amount of time taken to generate the electronic activity using the language complexity score and the character count or word count. The method may include generating, by the one or more processors, the QoE score corresponding to the estimated quality based on the estimated amount of time. The method may include storing, by the one or more processors, in a data structure, an association between the electronic activity and the QoE score.

Yet another aspect of the present disclosure relates to a non-transitory computer-readable storage medium having instructions embodied thereon, the instructions being executable by one or more processors to perform a method. The method may include identifying, by one or more processors, an electronic activity identifying a sender and one or more recipients and a body including content. The method may include determining, by the one or more processors, using a quality estimation model, a quality of electronic activity score corresponding to an estimated quality of the electronic activity. The determining may further include. The method may include parsing, by the one or more processors, the content of the electronic activity. The method may include generating, by the one or more processors, using a language complexity determination engine, a language complexity score indicating a level of language complexity. The method may include determining, by the one or more processors, a character count or word count. The method may include determining, by the one or more processors, an estimated amount of time taken to generate the electronic activity using the language complexity score and the character count or word count. The method may include generating, by the one or more processors, the QoE score corresponding to the estimated quality based on the estimated amount of time. The method may include storing, by the one or more processors, in a data structure, an association between the electronic activity and the QoE score.

One aspect of the present disclosure relates to a method for updating email addresses based on email generation patterns. The method may include maintaining, by one or more processors, a plurality of member node profiles corresponding to a plurality of unique member entities. Each member node profile includes a plurality of field-value pairs. The method may include accessing, by the one or more processors, a plurality of electronic activities transmitted or received via electronic accounts associated with one or more data source providers. The plurality of electronic activities are used to update the plurality of member node profiles. The method may include determining, by the one or more processors, for a group entity, a plurality of member node profiles linked to a group node profile of the group entity. The method may include identifying, by the one or more processors, for each member node profile of the plurality of member node profiles, a first field-value pair of the member node profile corresponding to an electronic account identifier of the member node profile. The electronic account identifier identifies an address via which electronic activities are transmitted from or received by the electronic account of the member node profile. The method may include determining, by the one or more processors, for each member node profile, a correlation between the first field-value pair corresponding to the electronic account identifier of the member node profile, a second field-value pair corresponding to a first name of the member node profile and a third field-value pair corresponding to a last name of the member node profile. The method may include determining, by the one or more processors, based on the determined correlation, an electronic account identifier pattern used by the group node to generate electronic account identifiers. The method may include storing, by the one or more processors, in one or more data structures, an association between the group node and the electronic account identifier pattern.

In some embodiments of the method, determining the correlation may include identifying, by the one or more processors, a second value of the second field-value pair identifying the first name of the member node profile. Determining the correlation may include identifying, by the one or more processors, a third value of the third field-value pair identifying the last name of the member node profile. Determining the correlation may include extracting, by the one or more processors, a character string preceding a predetermined character of the first value identifying the electronic account identifier. Determining the correlation may include comparing, by the one or more processors, the character string to the second value and the third value.

In some embodiments of the method, the electronic account identifier includes a first electronic account identifier. The method may further include accessing, by the one or more processors, a second electronic activity identifying a second electronic account identifier. The method may further include determining, by the one or more processors, that the second electronic account identifier corresponds to the group node. The method may further include determining, by the one or more processors, at least one of a first name or a last name based on the electronic account identifier pattern of the group node.

In some embodiments of the method, it may further include generating, by the one or more processors, a first activity field-value pair identifying the second electronic account identifier. The method may further include generating, by the one or more processors, a second activity field-value pair identifying the first name or last name determined from the second electronic account identifier.

The method may further include matching, by the one or more processors, the second electronic activity to a member node profile of the plurality of member node profiles based on comparing the second activity field-value pair to a corresponding node field-value pair of the member node profile.

In some embodiments of the method, it may further include generating, by the one or more processors, for a member node profile of the plurality of member node profiles that is linked to the group node profile, a first value corresponding to a first field of the member node profile corresponding to the first name or a second value corresponding to a second field of the member node profile corresponding to the last name by using the electronic account identifier pattern on the electronic account identifier stored as a value in a third field of the member node profile corresponding to the electronic account identifier of the member node profile.

In some embodiments of the method, determining, for the group entity, the plurality of member node profiles linked to the group entity may further include identifying, by the one or more processors, a value of a company name field of the member node profile, and determining, by the one or more processors, that the value of the company name field matches a value of a company name field of the group node profile of the group entity or a domain field of the group node profile.

In some embodiments of the method, it may further include determining a relationship between a first group entity and a second group entity. Determining the relationship may further include determining, by the one or more processors, for each of a plurality of member node profiles of the first group entity, that a first confidence score of a respective value including a first domain name of the member node profile is decreasing. Determining the relationship may further include determining, by the one or more processors, for each of the plurality of member node profiles of the first group entity, that a second confidence score of a respective value including a second domain name corresponding to the second group entity of the member node profile is increasing. Determining the relationship may further include determining, by the one or more processors, the relationship between the first group entity and a second group entity based on the first confidence score and the second confidence score.

In some embodiments of the method, the group entity is the second group entity. The method may further include generating, by the one or more processors, for a first member node profile of the plurality of member node profiles of the first group entity, a new electronic account identifier including the second domain name of the second group entity using the electronic account identifier pattern. The method may further include updating, by the one or more processors, the first member node profile of the plurality of member node profiles of the first group entity to include the new electronic account identifier as a value to the field corresponding to the electronic account identifier.

In some embodiments of the method, the group entity is a first group entity. The method may further include determining, by the one or more processors, that electronic activities identifying electronic account identifiers of the first group entity triggered generation of a first number of bounceback electronic activities within a predetermined time period. The method may further include determining, by the one or more processors, a second group entity from content included in the bounceback electronic activities. The method may further include determining, by the one or more processors, that the first number of bounceback electronic activities satisfies an entity change detection policy. The method may further include determining a relationship between the first entity and the second entity responsive to the entity change detection policy.

In some embodiments of the method, it may further include identifying, by the one or more processors, for each member node profile of the plurality of member node profiles, from a second value of the field corresponding to the electronic account identifier, a second domain name of the second value. The method may further include determining, by the one or more processors, that a number of member node profiles including the second domain name in the second value of the field corresponding to the electronic account identifier satisfies a threshold value. The method may further include determining, by the one or more processors, that the group entity owns the second domain name. The method may further include storing, by the one or more processors, an association between the group entity and the second domain name.

In some embodiments of the method, it may further include maintaining, by the one or more processors, at least one of a first name distribution based on the count of each node value of the plurality of node field-value pairs that is associated with a first name field or a last name distribution based on the count of each node value of the plurality of node field-value pairs that is associated with a last name field.

In some embodiments of the method, it may further include determining, by the one or more processors, a frequency score based on the at least one of the first name distribution or the last name distribution.

In some embodiments of the method, it may further include generating, by the one or more processors, the plurality of activity field-value pairs using at least one of the first name distribution or the last name distribution.

Another aspect of the present disclosure relates to a system configured to update email addresses based on email generation patterns. The system may include one or more hardware processors configured by machine-readable instructions. The processor(s) may be configured to maintain a plurality of member node profiles corresponding to a plurality of unique member entities.

Each member node profile includes a plurality of field-value pairs. The processor(s) may be configured to access a plurality of electronic activities transmitted or received via electronic accounts associated with one or more data source providers, the plurality of electronic activities used to update the plurality of member node profiles. The processor(s) may be configured to determine, for a group entity, a plurality of member node profiles linked to a group node profile of the group entity. The processor(s) may be configured to identify, for each member node profile of the plurality of member node profiles, a first field-value pair of the member node profile corresponding to an electronic account identifier of the member node profile, the electronic account identifier identifying an address via which electronic activities are transmitted from or received by the electronic account of the member node profile. The processor(s) may be configured to determine, for each member node profile, a correlation between the first field-value pair corresponding to the electronic account identifier of the member node profile, a second field-value pair corresponding to a first name of the member node profile and a third field-value pair corresponding to a last name of the member node profile. The processor(s) may be configured to determine, based on the determined correlation, an electronic account identifier pattern used by the group node to generate electronic account identifiers. The processor(s) may be configured to store, in one or more data structures, an association between the group node and the electronic account identifier pattern.

In some embodiments of the system, the processor(s) may be further configured to identify a second value of the second field-value pair identifying the first name of the member node profile. The processor(s) may be further configured to identify a third value of the third field-value pair identifying the last name of the member node profile. The processor(s) may be further configured to extract a character string preceding a predetermined character of the first value identifying the electronic account identifier. The processor(s) may be further configured to compare the character string to the second value and the third value.

In some embodiments of the system, the electronic account identifier includes a first electronic account identifier. The processor(s) may be further configured to access a second electronic activity identifying a second electronic account identifier. The processor(s) may be further configured to determine that the second electronic account identifier corresponds to the group node. The processor(s) may be further configured to determine at least one of a first name or a last name based on the electronic account identifier pattern of the group node.

In some embodiments of the system, the processor(s) may be further configured to generate a first activity field-value pair identifying the second electronic account identifier. The processor(s) may be further configured to generate a second activity field-value pair identifying the first name or last name determined from the second electronic account identifier. The processor(s) may be further configured to match the second electronic activity to a member node profile of the plurality of member node profiles based on comparing the second activity field-value pair to a corresponding node field-value pair of the member node profile.

In some embodiments of the system, the processor(s) may be further configured to generate, for a member node profile of the plurality of member node profiles that is linked to the group node profile, a first value corresponding to a first field of the member node profile corresponding to the first name or a second value corresponding to a second field of the member node profile corresponding to the last name by using the electronic account identifier pattern on the electronic account identifier stored as a value in a third field of the member node profile corresponding to the electronic account identifier of the member node profile.

In some embodiments of the system, the processor(s) may be further configured to identify a value of a company name field of the member node profile. The processor(s) may be further configured to determine that the value of the company name field matches a value of a company name field of the group node profile of the group entity or a domain field of the group node profile.

In some embodiments of the system, the processor(s) may be further configured to determine, for each of a plurality of member node profiles of a first group entity, that a first confidence score of a respective value including a first domain name of the member node profile is decreasing. The processor(s) may be further configured to determine, for each of the plurality of member node profiles of the first group entity, that a second confidence score of a respective value including a second domain name corresponding to a second group entity of the member node profile is increasing. The processor(s) may be further configured to determine the relationship between the first group entity and a second group entity based on the first confidence score and the second confidence score.

In some embodiments of the system, the group entity is the second group entity. The processor(s) may be further configured to generate, for a first member node profile of the plurality of member node profiles of the first group entity, a new electronic account identifier including the second domain name of the second group entity using the electronic account identifier pattern. The processor(s) may be further configured to update, the first member node profile of the plurality of member node profiles of the first group entity to include the new electronic account identifier as a value to the field corresponding to the electronic account identifier.

In some embodiments of the system, the group entity is a first group entity. The processor(s) may be further configured to determine that electronic activities identifying electronic account identifiers of the first group entity triggered generation of a first number of bounceback electronic activities within a predetermined time period. The processor(s) may be further configured to determine a second group entity from content included in the bounceback electronic activities. The processor(s) may be further configured to determine that the first number of bounceback electronic activities satisfies an entity change detection policy. The processor(s) may be further configured to determine a relationship between the first entity and the second entity responsive to the entity change detection policy.

In some embodiments of the system, the processor(s) may be further configured to identify, for each member node profile of the plurality of member node profiles, from a second value of the field corresponding to the electronic account identifier, a second domain name of the second value. The processor(s) may be further configured to determine that a number of member node profiles including the second domain name in the second value of the field corresponding to the electronic account identifier satisfies a threshold value. The processor(s) may be further configured to determine that the group entity owns the second domain name. The processor(s) may be further configured to store an association between the group entity and the second domain name.

Yet another aspect of the present disclosure relates to a non-transient computer-readable storage medium having instructions embodied thereon, the instructions being executable by one or more processors to perform a method for updating email addresses based on email generation patterns. The method may include maintaining, by one or more processors, a plurality of member node profiles corresponding to a plurality of unique member entities. Each member node profile includes a plurality of field-value pairs. The method may include accessing, by the one or more processors, a plurality of electronic activities transmitted or received via electronic accounts associated with one or more data source providers. The plurality of electronic activities are used to update the plurality of member node profiles. The method may include determining, by the one or more processors, for a group entity, a plurality of member node profiles linked to a group node profile of the group entity. The method may include identifying, by the one or more processors, for each member node profile of the plurality of member node profiles, a first field-value pair of the member node profile corresponding to an electronic account identifier of the member node profile. The electronic account identifier identifies an address via which electronic activities are transmitted from or received by the electronic account of the member node profile. The method may include determining, by the one or more processors, for each member node profile, a correlation between the first field-value pair corresponding to the electronic account identifier of the member node profile, a second field-value pair corresponding to a first name of the member node profile and a third field-value pair corresponding to a last name of the member node profile. The method may include determining, by the one or more processors, based on the determined correlation, an electronic account identifier pattern used by the group node to generate electronic account identifiers. The method may include storing, by the one or more processors, in one or more data structures, an association between the group node and the electronic account identifier pattern.

At least one aspect of the present disclosure relates to systems and methods for determining a preferred communication channel based on determining a status of a node profile using electronic activities. The method may include accessing, by the one or more processors, a plurality of electronic activities. The electronic activities may be transmitted or received via electronic accounts associated with one or more data source providers. The method may include maintaining, by one or more processors, a plurality of node profiles. The node profiles may correspond to a plurality of unique entities. Each node profile may include one or more node field-value pairs. The method may include identifying, by the one or more processors, a node profile from the plurality of node profiles for the at least one participant of each of the plurality of electronic activities. The method may include generating, by the one or more processors, for a node profile, based on a subset of the plurality of electronic activities linked to the node profile, a communication mode schedule identifying one or more communication modes over which to communicate with a first entity corresponding to the node profile at one or more time periods. The method may include transmitting, by the one or more processors, a first notification to a first device of the first entity via a communication mode selected using the communication mode schedule.

In some implementations of the method, the method may include generating the first notification. The first notification can identify an action to be performed and a recipient related to the action. The method may include determining a second communication mode schedule of the recipient based on electronic activities linked with a second node profile of the recipient. The method may include identifying, by the one or more processors, for a time period, a communication mode via which to communicate with the recipient. The method may include selecting, by the one or more processors, an electronic activity type of the electronic activity based on the communication mode via which to communicate with the recipient. The method may include identifying the electronic activity type in the first notification.

In some implementations of the method, the method may include identifying, by the one or more processors, a plurality of electronic activities linked to the first node profile. The method may include identifying, for each electronic activity of the plurality of electronic activities, an electronic activity type. The method may include generating, by the one or more processors, an availability schedule of the first node profile based on the plurality of electronic activities. The availability schedule can indicate time frames that a user of the first device is available. The method may include identifying, by the one or more processors, a plurality of electronic activities linked to the first node profile. The method may include determining, from the plurality of electronic activities, a response rate for each type of electronic activity. The method may include using the response rate to select a communication mode channel via which to communicate. The method may include selecting, from a plurality of devices including the first device, the first device to which to transmit the notification based on the communication mode schedule of the first node profile.

In some implementations of the method, the method may include identifying, by the one or more processors, a node profile of a second entity. The method may include identifying, by the one or more processors, a plurality of electronic activities linked to the node profile of the second entity. The method may include determining, by the one or more processors, for each of a plurality of electronic activity types, a response rate based on the plurality of electronic activities. The method may include selecting, by the one or more processors, a second communication mode for transmission of a second notification to the second device based on the response rate of each of the plurality of electronic activity types. In some implementations of the method, the method may include identifying, by the one or more processors and based on the node profile, a record object of a plurality of record objects of one or more systems of record. Each of the plurality of record objects can include one or more object fields having one or more object field value pairs. The method may include determining, by the one or more processors, an action to perform to increase a completion score of the record object. The method may include selecting, by the one or more processors, the communication mode based on the action to be performed to increase the completion score of the record object.

At least one aspect of the present disclosure relates to a system for determining a preferred communication channel based on determining a status of a node profile using electronic activities. The system may include one or more hardware processors configured by machine-readable instructions. The processor(s) may be configured to access a plurality of electronic activities transmitted or received via electronic accounts associated with one or more data source providers. The processor(s) may be configured to maintain a plurality of node profiles corresponding to a plurality of unique entities, each node profile including one or more node field-value pairs. The processor(s) may be configured to identify a node profile from the plurality of node profiles for the at least one participant of each of the plurality of electronic activities. The processor(s) may be configured to generate, for a node profile, based on a subset of the plurality of electronic activities linked to the node profile, a communication mode schedule identifying one or more communication modes over which to communicate with a first entity corresponding to the node profile at one or more time periods. The processor(s) may be configured to transmit a first notification to a first device of the first entity via a communication mode selected using the communication mode schedule.

In some implementations of the system, the processor(s) may be configured to generate the first notification, the first notification identifying an action to be performed and a recipient related to the action. In some implementations of the system, the processor(s) may be configured to determine a second communication mode schedule of the recipient based on electronic activities linked with a second node profile of the recipient. The processor(s) may be configured to identify, for a time period, a communication mode via which to communicate with the recipient. The processor(s) may be configured to select an electronic activity type of the electronic activity based on the communication mode via which to communicate with the recipient. The processor(s) may be configured to identify the electronic activity type in the first notification. In some implementations of the system, the processor(s) may be configured to identify a plurality of electronic activities linked to the first node profile. The processor(s) may be configured to identify, for each electronic activity of the plurality of electronic activities, an electronic activity type. The processor(s) may be configured to generate an availability schedule of the first node profile based on the plurality of electronic activities, the availability schedule indicating time frames that a user of the first device is available.

In some implementations of the system, the processor(s) may be configured to identify a plurality of electronic activities linked to the first node profile. The processor(s) may be configured to determine, from the plurality of electronic activities, a response rate for each type of electronic activity. The processor(s) may be configured to use the response rate to select a communication mode channel via which to communicate. In some implementations of the system, the processor(s) may be configured to select, from a plurality of devices including the first device, the first device to which to transmit the notification based on the communication mode schedule of the first node profile. In some implementations of the system, the processor(s) may be configured to identify a node profile of a second entity. The processor(s) may be configured to identify a plurality of electronic activities linked to the node profile of the second entity. The processor(s) may be configured to determine, for each of a plurality of electronic activity types, a response rate based on the plurality of electronic activities. The processor(s) may be configured to select a second communication mode for transmission of a second notification to the second device based on the response rate of each of the plurality of electronic activity types.

In some implementations of the system, the processor(s) may be configured to identify, based on the node profile, a record object of a plurality of record objects of one or more systems of record. Each of the plurality of record objects can include one or more object fields having one or more object field value pairs. The processor(s) may be configured to determine an action to perform to increase a completion score of the record object. The processor(s) may be configured to select the communication mode based on the action to be performed to increase the completion score of the record object.

At least one aspect of the present disclosure relates to anon-transient computer-readable storage medium having instructions embodied or stored thereon. The instructions can be executed by one or more processors access a plurality of electronic activities transmitted or received via electronic accounts associated with one or more data source providers. The processor(s) may be configured to maintain a plurality of node profiles corresponding to a plurality of unique entities, each node profile including one or more node field-value pairs. The processor(s) may be configured to identify a node profile from the plurality of node profiles for the at least one participant of each of the plurality of electronic activities. The processor(s) may be configured to generate, for a node profile, based on a subset of the plurality of electronic activities linked to the node profile, a communication mode schedule identifying one or more communication modes over which to communicate with a first entity corresponding to the node profile at one or more time periods. The processor(s) may be configured to transmit a first notification to a first device of the first entity via a communication mode selected using the communication mode schedule.

In some implementations, the non-transitory computer-readable storage medium can include the processor(s) to generate the first notification, the first notification identifying an action to be performed and a recipient related to the action. The processor(s) may be configured to generate the first notification, the first notification identifying an action to be performed and a recipient related to the action. The processor(s) may be configured to determine a second communication mode schedule of the recipient based on electronic activities linked with a second node profile of the recipient. The processor(s) may be configured to identify, for a time period, a communication mode via which to communicate with the recipient. The processor(s) may be configured to select an electronic activity type of the electronic activity based on the communication mode via which to communicate with the recipient. The processor(s) may be configured to identify the electronic activity type in the first notification. The processor(s) may be configured to generate the first notification, the first notification identifying an action to be performed and a recipient related to the action. The processor(s) may be configured to identify a plurality of electronic activities linked to the first node profile. The processor(s) may be configured to identify, for each electronic activity of the plurality of electronic activities, an electronic activity type. The processor(s) may be configured to generate an availability schedule of the first node profile based on the plurality of electronic activities, the availability schedule indicating time frames that a user of the first device is available.

At least one aspect of this disclosure is directed to a method for inferring a time zone ofa node profile using electronic activities. The method can include accessing, by one or more processors, a plurality of electronic activities transmitted or received via a plurality of electronic accounts associated with a data source provider. The method can include identifying, by the one or more processors, for a node profile maintained by the one or more processors, a set of electronic activities sent from or received by an electronic account of the plurality of electronic accounts linked to the node profile within a time period. The method can include identifying, by the one or more processors, for each electronic activity of the set of electronic activities, a timestamp at which the electronic activity was sent or received. The method can include generating, by the one or more processors, for each of a plurality of time intervals within the time period, a temporal distribution of electronic activity for the time interval based on respective timestamps of each electronic activity of the set of electronic activities within the time interval. The method can include determining, by the one or more processors, a time zone of the node profile based on the temporal distribution of electronic activity. The method can include storing, by the one or more processors in one or more data structures, an association between the determined time zone and the node profile.

In some implementations, the method can include receiving, by the one or more processors, an electronic activity not included in the set of electronic activities. In some implementations, the method can include parsing, by the one or more processors, the electronic activity to identify a participant and a timestamp. In some implementations, the method can include determining, by the one or more processors, a plurality of candidate node profiles, based on the participant. In some implementations, the method can include selecting, by the one or more processors, one of the plurality of candidate node profiles to match with the electronic activity based on a time zone of selected candidate node profile and the timestamp of the electronic activity.

In some implementations, the temporal distribution can be a first temporal distribution. In some implementations, determining the time zone of the node profile can include identifying, by the one or more processors, a plurality of second temporal distributions each associated with a respective predetermined time zone. In some implementations, the method can include selecting, by the one or more processors, one of the plurality of second temporal distributions based on a comparison with the first temporal distribution. In some implementations, the method can include determining, by the one or more processors, the time zone based on the respective predetermined time zone of the selected second temporal distribution.

In some implementations, each second temporal distribution can be generated based on a respective second set of electronic activities of one or more second node profiles. In some implementations, each second node profile can be associated with the respective predetermined time zone.

In some implementations, the method can include identifying, by the one or more processors, a system of record having at least one record object associated with the node profile. In some implementations, the method can include transmitting, by the one or more processors, an instruction to cause the system of record to update a time zone field-value pair of the at least one record object corresponding to the node profile.

In some implementations, the method can include updating, by the one or more processors, a time zone field-value pair of the node profile to match the determined time zone.

In some implementations, the method can include generating, by the one or more processors, a notification for a user associated with the node profile. In some implementations, the method can include scheduling, by the one or more processors, transmission of the notification to a computing device of the user associated with the node profile based on the determined time zone. In some implementations, scheduling transmission of the notification can include identifying, by the one or more processors, a restricted notification period based on the determined time zone. In some implementations, the method can include restricting, by the one or more processors, transmission of the notification to the computing device of the user associated with the node profile during the restricted notification period.

In some implementations, the method can include parsing, by the one or more processors, a first electronic activity of the set of electronic activities to identify a character string corresponding to a geographic location. In some implementations, the method can include determining, by the one or more processors, the time zone based on the geographic location. In some implementations, parsing the first electronic activity of the set of electronic activities to identify the character string corresponding to the geographic location can include extracting, by the one or more processors, the geographic location from a body of the first electronic activity.

In some implementations, identifying the set of electronic activities can include selecting each electronic activity to include in the set of electronic activities responsive to determining that the electronic activity is of a first electronic activity type.

In some implementations, the method can include comparing, by the one or more processors, the time zone with a value of a field-value pair of the node profile corresponding to a location. In some implementations, the method can include updating, by the one or more processors, a confidence score of the value of the field-value pair of the node profile corresponding to the location, based on the comparison.

In some implementations, determining the time zone of the node profile based on the temporal distribution of electronic activity can further include determining, by the one or more processors, that the temporal distribution of electronic activity corresponds to the time zone of the node profile using a machine learning model trained to classify temporal distributions of electronic activity.

At least another aspect of this disclosure is directed to a system for inferring a time zone of a node profile using electronic activities. The system can include one or more hardware processors configured by machine-readable instructions. The one or more hardware processors can be configured to access a plurality of electronic activities transmitted or received via a plurality of electronic accounts associated with a data source provider. The one or more hardware processors can be configured to identify, for a node profile maintained by the one or more processors, a set of electronic activities sent from or received by an electronic account of the plurality of electronic accounts linked to the node profile within a time period. The one or more hardware processors can be configured to identify, for each electronic activity of the set of electronic activities, a timestamp at which the electronic activity was sent or received. The one or more hardware processors can be configured to generate, for each of a plurality of time intervals within the time period, a temporal distribution of electronic activity for the time interval based on respective timestamps of each electronic activity of the set of electronic activities within the time interval. The one or more hardware processors can be configured to determine a time zone of the node profile based on the temporal distribution of electronic activity. The one or more hardware processors can be configured to store, in one or more data structures, an association between the determined time zone and the node profile.

In some implementations, the one or more hardware processors can be further configured to receive an electronic activity not included in the set of electronic activities. In some implementations, the one or more hardware processors can be further configured to parse the electronic activity to identify a participant and a timestamp. In some implementations, the one or more hardware processors can be further configured to determine a plurality of candidate node profiles, based on the participant. In some implementations, the one or more hardware processors can be further configured to select one of the plurality of candidate node profiles to match with the electronic activity based on a time zone of selected candidate node profile and the timestamp of the electronic activity.

In some implementations, the temporal distribution can be a first temporal distribution. In some implementations, the one or more hardware processors can be further configured to determine the time zone of the node profile by identifying a plurality of second temporal distributions each associated with a respective predetermined time zone. In some implementations, the one or more hardware processors can be further configured to select one of the plurality of second temporal distributions based on a comparison with the first temporal distribution. In some implementations, the one or more hardware processors can be further configured to determine the time zone based on the respective predetermined time zone of the selected second temporal distribution. In some implementations, the one or more hardware processors are further configured by the machine readable instructions to generate each second temporal distribution based on a respective second set of electronic activities of one or more second node profiles. In some implementations, each second node profile can be associated with the respective predetermined time zone.

In some implementations, the one or more hardware processors can be further configured to identify a system of record having at least one record object associated with the node profile. In some implementations, the one or more hardware processors can be further configured to transmit an instruction to cause the system of record to update a time zone field-value pair of the at least one record object corresponding to the node profile.

In some implementations, the one or more hardware processors are further configured by the machine readable instructions to update a time zone field-value pair of the node profile to match the determined time zone.

In some implementations, the one or more hardware processors can be further configured to generate a notification for a user associated with the node profile. In some implementations, the one or more hardware processors can be further configured to schedule transmission of the notification to a computing device of the user associated with the node profile based on the determined time zone.

In some implementations, the one or more hardware processors are further configured by the machine readable instructions to schedule transmission of the notification by identifying, by the one or more processors, a restricted notification period based on the determined time zone. In some implementations, the one or more hardware processors can be further configured to restrict transmission of the notification to the computing device of the user associated with the node profile during the restricted notification period.

At least another aspect of this disclosure is directed to a non-transient computer-readable storage medium having instructions embodied thereon. The instructions can be executable by one or more processors to perform a method for inferring a time zone of a node profile using electronic activities. The method can include accessing a plurality of electronic activities transmitted or received via a plurality of electronic accounts associated with a data source provider. The method can include identifying, for a node profile maintained by the one or more processors, a set of electronic activities sent from or received by an electronic account of the plurality of electronic accounts linked to the node profile within a time period. The method can include identifying, for each electronic activity of the set of electronic activities, a timestamp at which the electronic activity was sent or received. The method can include generating, for each of a plurality of time intervals within the time period, a temporal distribution of electronic activity for the time interval based on respective timestamps of each electronic activity of the set of electronic activities within the time interval. The method can include determining a time zone of the node profile based on the temporal distribution of electronic activity. The method can include storing, in one or more data structures, an association between the determined time zone and the node profile.

One aspect of this disclosure is directed to a method for determining domain names of a group entity using electronic activities and systems of record. The method can include maintaining, by one or more processors, a plurality of node profiles including one or more node field-value pairs. Each node profile of the plurality of node profiles can include a first field-value pair identifying a group entity. The method can include identifying, by the one or more processors, from first electronic account identifiers included as values of a respective electronic account identifier field of the plurality of node profiles, a first domain name corresponding to the group entity. The method can include accessing, by the one or more processors, a plurality of data points corresponding to a subset of the plurality of node profiles. Each data point can identify a respective second electronic account identifier of a respective node profile of the subset. Each respective second electronic account identifier can have a second domain name. The method can include updating, by the one or more processors, each respective node profile of the subset to include the respective second electronic account identifier corresponding to the node profile. The method can include determining, by the one or more processors, that the subset of the plurality of node profiles that was updated to include the respective second electronic account identifiers satisfies a domain name update policy. The method can include storing, by the one or more processors, in at least one first data structure, an association between the group entity, the first domain name, and the second domain name.

In some implementations, the plurality of data points can include a plurality of electronic activities. In some implementations, the method can include matching, by the one or more processors, each electronic activity of the plurality of electronic activities with one or more node profiles of the plurality of node profiles by comparing activity field-value pairs of each electronic activity with node field-value pairs of the node profile. In some implementations, updating each respective node profile of the subset to include the respective second electronic account identifier can include updating, by the one or more processors, each respective node profile of the subset responsive to the matching.

In some implementations, the method can include selecting, by the one or more processors, an update time interval. In some implementations, determining that the subset of the plurality of node profiles that was updated to include the respective second electronic account identifiers satisfies a domain name update policy can include determining, by the one or more processors, that each node profile of the subset of the plurality of node profiles was updated to include the respective second electronic account identifier during the update time interval.

In some implementations, the group entity can include a first group entity. In some implementations, the method can include accessing, by the one or more processors, a plurality of electronic activities sent or received by the subset of the plurality of node profiles. In some implementations, the method can include parsing, by the one or more processors, the plurality of electronic activities to identify a relationship between the group entity and a second group entity corresponding to the second domain name. In some implementations, the method can include storing, by the one or more processors, in at least one second data structure, an association between the first group entity and the second group entity.

In some implementations, the method can include determining, by the one or more processors, that the relationship between the first group node and the second group nodes satisfies a hierarchy update policy. In some implementations, the method can include updating, by the one or more processors, a node graph including the first group node and the second group node to include a connection between the first group node and the second group node. In some implementations, the connection can indicate the relationship between the first group node and the second group nodes.

In some implementations, the method can include determining, by the one or more processors, that the relationship between the first group node and the second group nodes satisfies a hierarchy update policy. In some implementations, the method can include identifying, by the one or more processors, a record object corresponding to at least one of the first group node or the second group node. In some implementations, the record object can be linked with an account hierarchy data structure. In some implementations, the method can include updating, by the one or more processors, the account hierarchy data structure responsive to determining that the relationship between the first group node and the second group nodes satisfies the hierarchy update policy.

In some implementations, the method can include accessing, by the one or more processors, a first electronic activity comprising a bounceback electronic activity. In some implementations, the method can include identifying, by the one or more processors, a second electronic activity that triggered the bounceback electronic activity. In some implementations, the method can include determining, by the one or more processors, that an intended recipient of the second electronic activity has an electronic account identifier associated with the first domain name. In some implementations, the method can include updating, by the one or more processors, a confidence score of an electronic account identifier value associated with the first domain name of a node profile corresponding to the intended recipient.

In some implementations, the method can include determining, by the one or more processors, for each node profile of the subset, a correlation between a first field-value pair corresponding to the second electronic account identifier of the node profile, a second field-value pair corresponding to a first name of the node profile, and a third field-value pair corresponding to a last name of the node profile. In some implementations, the method can include determining, by the one or more processors, based on the correlation, an electronic account identifier pattern of the second electronic account identifiers. In some implementations, the method can include storing, by the one or more processors, in one or more second data structures, an association between the group entity and the electronic account identifier pattern.

In some implementations, the method can include generating, by the one or more processors, for a first node profile of the plurality of node profiles that is not included in the subset, a new electronic account identifier including the second domain name using the electronic account identifier pattern. In some implementations, the method can include updating, by the one or more processors, the first node profile to include the new electronic account identifier.

In some implementations, the method can include accessing, by the one or more processors, an electronic activity identifying a participant having an electronic account identifier associated with the second domain name. In some implementations, the method can include determining, by the one or more processors, at least one of a first name or a last name of the participant based on the electronic account identifier of the electronic activity and the electronic activity identifier pattern. In some implementations, the method can include generating, by the one or more processors, an activity field-value pair identifying the first name or last name of the participant. In some implementations, the method can include matching, by the one or more processors, the electronic activity to a first node profile of the plurality of node profiles based on comparing the activity field-value pair to a corresponding node field-value pair of the first node profile. In some implementations, the first node profile can correspond to the participant.

In some implementations, the second domain name can correspond to a second group entity. In some implementations, the method can include identifying, by the one or more processors, a system of record associated with a data source provider corresponding to a third group entity. In some implementations, the method can include identifying, by the one or more processors, a record object included in the system of record having an object field-value pair corresponding to the first domain name. In some implementations, the method can include updating, by the one or more processors, the record object to include a value corresponding to the second domain name.

Another aspect of this disclosure is directed to a system for determining domain names of a group entity using electronic activities and systems of record. The system can include one or more hardware processors configured by machine-readable instructions to maintain a plurality of node profiles including one or more node field-value pairs, each node profile of the plurality of node profiles including a first field-value pair identifying a group entity. The one or more hardware processors can be configured by the machine-readable instructions to identify, from first electronic account identifiers included as values of a respective electronic account identifier field of the plurality of node profiles, a first domain name corresponding to the group entity. The one or more hardware processors can be configured by the machine-readable instructions to access a plurality of data points corresponding to a subset of the plurality of node profiles. Each data point can identify a respective second electronic account identifier of a respective node profile of the subset. Each respective second electronic account identifier can have a second domain name. The one or more hardware processors can be configured by the machine-readable instructions to update each respective node profile of the subset to include the respective second electronic account identifier corresponding to the node profile. The one or more hardware processors can be configured by the machine-readable instructions to determine that the subset of the plurality of node profiles that was updated to include the respective second electronic account identifiers satisfies a domain name update policy. The one or more hardware processors can be configured by the machine-readable instructions to store, in at least one first data structure, an association between the group entity, the first domain name, and the second domain name.

In some implementations, the plurality of data points can include a plurality of electronic activities. In some implementations, the one or more hardware processors can be configured by the machine readable instructions to match each electronic activity of the plurality of electronic activities with one or more node profiles of the plurality of node profiles by comparing activity field-value pairs of each electronic activity with node field-value pairs of the node profile. In some implementations, the one or more hardware processors can be configured by the machine-readable instructions to update each respective node profile of the subset to include the respective second electronic account identifier responsive to the matching.

In some implementations, the one or more hardware processors can be further configured by the machine readable instructions to select an update time interval. In some implementations, the one or more hardware processors can be configured by the machine-readable instructions to determine that each node profile of the subset of the plurality of node profiles was updated to include the respective second electronic account identifier during the update time interval.

In some implementations, the group entity can include a first group entity. In some implementations, the one or more hardware processors can be further configured by the machine readable instructions to access a plurality of electronic activities sent or received by the subset of the plurality of node profiles. In some implementations, the one or more hardware processors can be configured by the machine-readable instructions to parse the plurality of electronic activities to identify a relationship between the group entity and a second group entity corresponding to the second domain name. In some implementations, the one or more hardware processors can be configured by the machine-readable instructions to store, in at least one second data structure, an association between the first group entity and the second group entity.

In some implementations, the one or more hardware processors can be further configured by the machine readable instructions to determine that the relationship between the first group node and the second group nodes satisfies a hierarchy update policy. In some implementations, the one or more hardware processors can be configured by the machine-readable instructions to update a node graph including the first group node and the second group node to include a connection between the first group node and the second group node. In some implementations, the connection can indicate the relationship between the first group node and the second group nodes.

In some implementations, the one or more hardware processors are further configured by the machine readable instructions to determine that the relationship between the first group node and the second group nodes satisfies a hierarchy update policy. In some implementations, the one or more hardware processors can be configured by the machine-readable instructions to identify a record object corresponding to at least one of the first group node or the second group node. In some implementations, the record object can be linked with an account hierarchy data structure. In some implementations, the one or more hardware processors can be configured by the machine-readable instructions to update the account hierarchy data structure responsive to determining that the relationship between the first group node and the second group nodes satisfies the hierarchy update policy.

In some implementations, the one or more hardware processors can be further configured by the machine readable instructions to access a first electronic activity comprising a bounceback electronic activity. In some implementations, the one or more hardware processors can be configured by the machine-readable instructions to identify a second electronic activity that triggered the bounceback electronic activity. In some implementations, the one or more hardware processors can be configured by the machine-readable instructions to determine that an intended recipient of the second electronic activity has an electronic account identifier associated with the first domain name. In some implementations, the one or more hardware processors can be configured by the machine-readable instructions to update a confidence score of an electronic account identifier value associated with the first domain name of a node profile corresponding to the intended recipient.

In some implementations, the one or more hardware processors can be further configured by the machine readable instructions to determine, for each node profile of the subset, a correlation between a first field-value pair corresponding to the second electronic account identifier of the node profile, a second field-value pair corresponding to a first name of the node profile, and a third field-value pair corresponding to a last name of the node profile. In some implementations, the one or more hardware processors can be configured by the machine-readable instructions to determine, based on the correlation, an electronic account identifier pattern of the second electronic account identifiers. In some implementations, the one or more hardware processors can be configured by the machine-readable instructions to store, in one or more second data structures, an association between the group entity and the electronic account identifier pattern.

Another aspect of this disclosure is directed to a non-transient computer-readable storage medium having instructions embodied thereon. The instructions can be executable by one or more processors to perform a method for determining domain names of a group entity using electronic activities and systems of record. The method can include maintaining a plurality of node profiles including one or more node field-value pairs. Each node profile of the plurality of node profiles can include a first field-value pair identifying a group entity. The method can include identifying, from first electronic account identifiers included as values of a respective electronic account identifier field of the plurality of node profiles, a first domain name corresponding to the group entity. The method can include accessing a plurality of data points corresponding to a subset of the plurality of node profiles. Each data point can identify a respective second electronic account identifier of a respective node profile of the subset. Each respective second electronic account identifier can have a second domain name. The method can include updating each respective node profile of the subset to include the respective second electronic account identifier corresponding to the node profile. The method can include determining that the subset of the plurality of node profiles that was updated to include the respective second electronic account identifiers satisfies a domain name update policy. The method can include storing, in at least one first data structure, an association between the group entity, the first domain name, and the second domain name.

### BRIEF DESCRIPTIONS OF THE DRAWINGS

FIG. 1 illustrates a tiered system architecture for aggregating electronic activities and synchronizing the electronic activities to one or more systems of record according to embodiments of the present disclosure;

FIG. 2 illustrates a process flow for aggregating electronic activities and synchronizing the electronic activities to one or more systems of record according to embodiments of the present disclosure;

FIG. 3 illustrates a processing flow diagram for aggregating electronic activities and synchronizing the electronic activities to one or more systems of record according to embodiments of the present disclosure;

FIG. 4 illustrates a node graph generation system for constructing a node graph based on electronic activity according to embodiments of the present disclosure;

FIGS. 5A-5C illustrate various types of example electronic activities according to embodiments of the present disclosure;

FIG. 6A illustrates a representation of a node profile of a node according to embodiments of the present disclosure;

FIG. 6B illustrates representations of three electronic activities and representations of three states of a node profile of a node according to embodiments of the present disclosure;

FIG. 7 illustrates a series of electronic activities between two nodes according to embodiments of the present disclosure;

FIG. 8 illustrates electronic activities involving two nodes and the impact a time decaying score has on the connection strength between the two nodes according to embodiments of the present disclosure;

FIG. 9 illustrates a block diagram of an example electronic activity linking engine according to embodiments of the present disclosure;

FIG. 10 illustrates a plurality of example record objects, and their interconnections, according to embodiments of the present disclosure;

FIG. 11 illustrates the restriction of a first grouping of record objects with a second grouping of record objects according to embodiments of the present disclosure;

FIG. 12 illustrates the application of a plurality of matching strategies and then pruning of the matched record objects with a second plurality of matching strategies according to embodiments of the present disclosure;

FIG. 13 illustrates an example calculation for calculating the engagement score of an opportunity record object according to embodiments of the present disclosure;

FIG. 14 illustrates an example user interface identifying various pieces of information that can be extracted from an electronic activity according to embodiments of the present disclosure;

FIG. 15 illustrates an example user interface identifying a record object corresponding to an opportunity according to embodiments of the present disclosure;

FIG. 16 illustrates a block diagram of an example process flow for processing electronic activities in a single-tenant configuration according to embodiments of the present disclosure;

FIG. 17 illustrates a block diagram of an example process flow for processing electronic activities in a multi-tenant configuration according to embodiments of the present disclosure;

FIG. 18 illustrates a block diagram of an example process flow for matching electronic activities with record objects according to embodiments of the present disclosure;

FIG. 19 illustrates a block diagram of an example method to match electronic activities directly to record objects according to embodiments of the present disclosure;

FIG. 20 illustrates a block diagram of an example process flow for matching electronic activities with record objects according to embodiments of the present disclosure;

FIG. 21 illustrates a block diagram of an example method to match electronic activities with record objects according to embodiments of the present disclosure;

FIG. 22 illustrates a block diagram of an example process to match electronic activities with node profiles according to embodiments of the present disclosure;

FIG. 23 illustrates a block diagram of an example method to match electronic activities with node profiles according to embodiments of the present disclosure;

FIG. 24 illustrates a block diagram of an example method to match electronic objects with node profiles according to embodiments of the present disclosure;

FIG. 25 illustrates a block diagram of a series of electronic activities between two nodes according to embodiments of the present disclosure;

FIG. 26 illustrates a representation of a node profile of a node according to embodiments of the present disclosure;

FIG. 27 illustrates a block diagram of an example method to generate confidence scores of values of fields based on data points according to embodiments of the present disclosure;

FIG. 28 illustrates a block diagram of a system for detecting events based on updates to node profiles from electronic activities;

FIG. 29 illustrates a block diagram of a method for detecting events based on updates to node profiles from electronic activities;

FIG. 30 illustrates a use case diagram of a user associated with a node profile engaged in a plurality of electronic activities in a time interval;

FIG. 31 illustrates an example graph showing a plurality of graphical representations of temporal distributions of electronic activity at various time intervals;

FIG. 32 illustrates an example method for inferring schedule patterns using electronic activities of node profiles;

FIG. 33 illustrates an example data structure configured for classifying phone numbers based on node profile data according to embodiments of the present disclosure;

FIG. 34 illustrates a method for classifying phone numbers based on node profile data according to embodiments of the present disclosure;

FIG. 35 illustrates a block diagram of one or more tags being associated with an event electronic based on a number of electronic activities according to embodiments of the present disclosure;

FIG. 36 illustrates an example method for confirming a scheduled event based on one or more electronic activities according to embodiments of the present disclosure;

FIG. 37 illustrates a block diagram of a series of electronic activities according to embodiments of the present disclosure;

FIG. 38 illustrates an example method for updating a node profile according to embodiments of the present disclosure;

FIG. 39 illustrates a block diagram of an example process flow for updating email addresses based on email generation patterns according to embodiments of the present disclosure;

FIG. 40 illustrates an example method for updating email addresses based on email generation patterns according to embodiments of the present disclosure;

FIG. 41 illustrates an example system for determining a preferred communication channel based on determining a status of a node profile using electronic activities according to embodiments of the present disclosure;

FIG. 42 illustrates a block diagram of an example method determining a preferred communication channel based on determining a status of a node profile using electronic activities according to embodiments of the present disclosure;

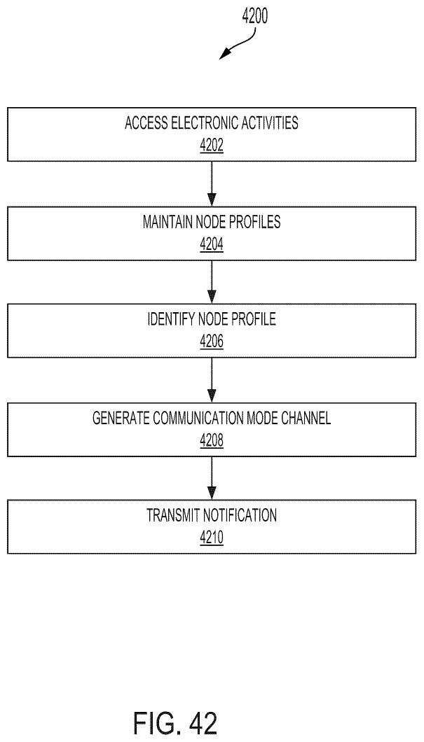

FIG. 43 illustrates a use case diagram of an example system for inferring a time zone of a node profile using electronic activities;

FIG. 44 illustrates an example graph showing a plurality of graphical representations of temporal distributions of electronic activity at various time intervals;

FIG. 45 illustrates a flow diagram of an example method for inferring a time zone of a node profile using electronic activities;

FIG. 46 illustrates a block diagram of a system for determining domain names of a group entity using electronic activities and systems of record, according to embodiments of the present disclosure;

FIG. 47 illustrates a flow diagram of an example method for determining domain names of a group entity using electronic activities and systems of record, according to embodiments of the present disclosure; and

FIG. 48 illustrates a simplified block diagram of a representative server system and client computer system according to embodiments of the present disclosure.

### DETAILED DESCRIPTION

The present disclosure relates to systems and methods for constructing a node graph based on electronic activity. The node graph can include a plurality of nodes and a plurality of edges between the nodes indicating activity or relationships that are derived from a plurality of data sources that can include one or more types of electronic activities. The plurality of data sources can include email or messaging servers, phone servers, servers storing calendar information, meeting information, among others. The plurality of data sources further includes systems of record, such as customer relationship management systems, enterprise resource planning systems, document management systems, applicant tracking systems or other source of data that may maintain electronic activities, activities or records.

The present disclosure further relates to systems and methods for using the node graph to manage, maintain, improve, or otherwise modify one or more systems of record by linking and or synchronizing electronic activities to one or more record objects of the systems of record. In particular, the systems described herein can be configured to automatically synchronize real-time or near real-time electronic activity to one or more objects of systems of record. The systems can further extract business process information from the systems of record and in combination with the node graph, use the extracted business process information to improve business processes and to provide data driven solutions to improve such business processes.

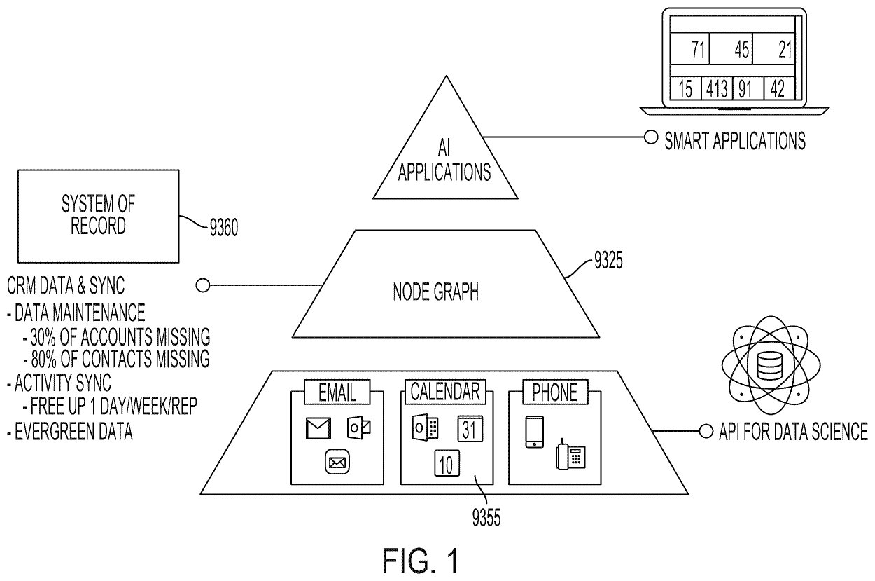

Referring briefly to FIG. 1, FIG. 1 illustrates a tiered system architecture for aggregating electronic activities and synchronizing the electronic activities to one or more systems of record according to embodiments of the present disclosure. As shown in FIG. 1, at the first tier, the system aggregates electronic activities from one or more data source providers. At the second tier, the system extracts information from the aggregated electronic activities and one or more systems of record of one or more data source providers to construct and maintain a node graph including the plurality of nodes and edges indicating the connections between the nodes. At the third tier, the system utilizes the electronic activities, the systems of record, and the node graph to provide data driven insights to improve one or more business processes of the data source providers and to assist various data source providers in extracting data driven insights.

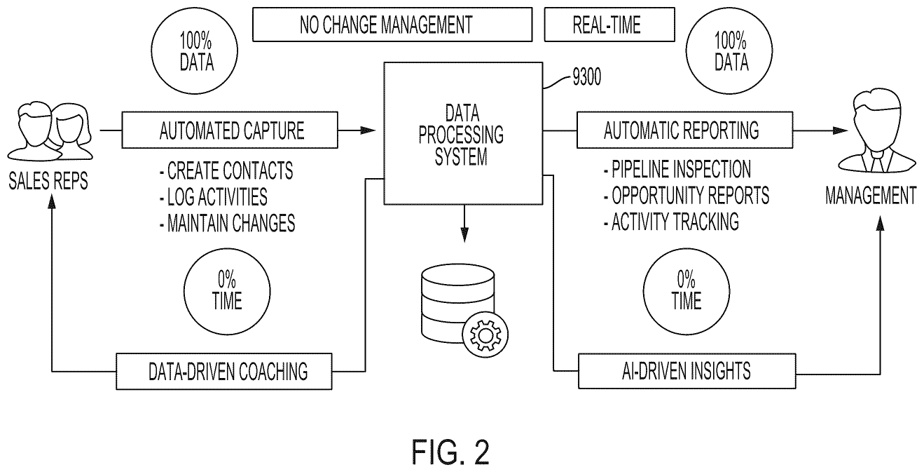

FIG. 2 illustrates a process flow for aggregating electronic activities and synchronizing the electronic activities to one or more systems of record according to embodiments of the present disclosure. The system can be configured to receive and aggregate electronic activities identifying one or more nodes. The system can parse the electronic activities and extract information from the electronic activities to generate node profiles for each node, log activities and maintain changes made to each of the node profiles maintained by the system. The system can further be configured to extract information from the electronic activities of the nodes and determine insights or metrics that can be shared with one or more other nodes and the users of the system. The system can be further configured to synchronize the electronic activities to objects of one or more systems of record.

In a particular use case, sales representatives of an organization may be involved in electronic activities, such as emails, phone calls, meetings, among others that can be tracked and captured by the system via ingestion from one or more data sources of the organization or other organizations. The system can extract information from the electronic activities that may be associated with deals or opportunities the sales representatives are working on. The system can use the information from these electronic activities to generate reports for managers of the organization. These reports are generated based on data derived from electronic activity without requiring the sales representatives to perform any additional activities. Furthermore, the managers also do not need to spend time generating these reports as the system can automatically generate these reports. Furthermore, the system can identify trends and behaviors that may be determined through machine learning techniques otherwise not tracked by the managers, thereby providing reports that may otherwise not be generated by the managers. Further, sales representatives may also no longer be required to spend time synchronizing electronic activities to one or more systems of record. Rather, the system can be configured to automatically synchronize the electronic activities to the appropriate objects of the one or more systems of record. The system can further receive information from the one or more systems and records to determine the results associated with the sales representative's efforts and perform analytics to generate recommendations to assist the sales representatives achieve their goals and eventually improve their performance as sales representatives as well as provide company management with recommendations about improving the performance of the overall business.

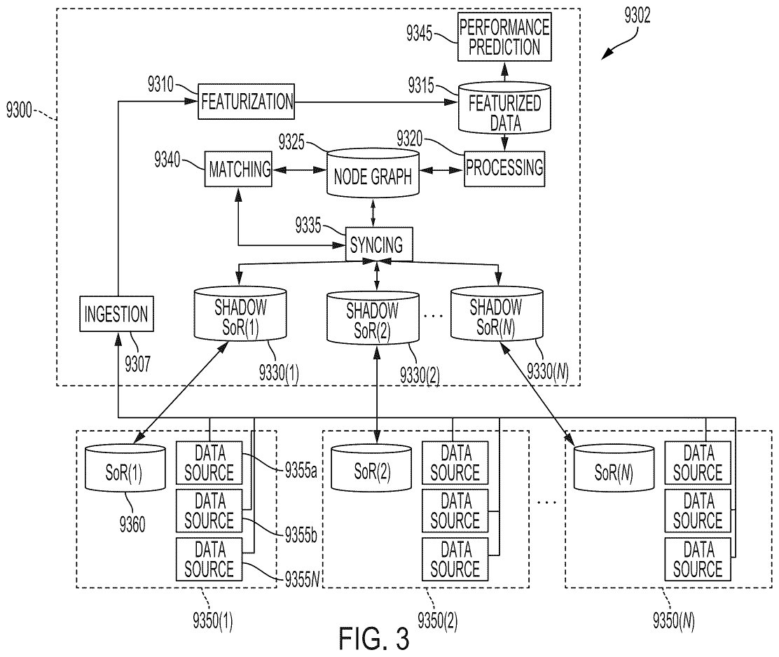

Referring now to FIG. 3, FIG. 3 illustrates a processing flow diagram for aggregating electronic activities, processing the electronic activities to update node profiles of people and to construct a node graph, and synchronizing the electronic activities to one or more systems of record. The process flow 9302 can be executed by a data processing system 9300 that can receive electronic activity and other data from a plurality of data source providers 9350a-n. Each data source provider 9350 can include one or more data sources 9355a-n and/or one or more system of record instances 9360. Examples of data sources can include electronic mail servers, telephone log servers, contact servers, other types of servers and end-user applications that may receive or maintain electronic activity data or profile data relating to one or more nodes. The data processing system 9300 can ingest electronic activity (9305). The data processing system 9300 can featurize and tag the ingested electronic activity (9310) and store the featurized data in a featurized data store (9315). The data processing system 9300 can process the featurized data (9320) to generate a node graph including a plurality of node profiles (9325). The data processing system 9300 can further maintain a plurality of system of record instances 9330a-n corresponding to system of record instances of the data source providers 9350. The data processing system 9300 can utilize the system of record instances to augment the node profiles of the node graph by synchronizing data stored in the system of record instances maintained by the data processing system (9335). The data processing system 9300 can further match the ingested electronic activities to one or more record objects maintained in one or more systems of record instances of the data source provider from which the electronic activity was received (9340). The data processing system 9300 can further synchronize the electronic activity matched to record objects to update the system of record instances of the data source provider (9335). Furthermore, the data processing system 9300 can use the featurized data to provide performance predictions and generate other business process related outputs, insights and recommendations (9345).

As described herein, electronic activity can include any type of electronic communication that can be stored or logged. Examples of electronic activity can include electronic mail messages, telephone calls, calendar invitations, social media messages, mobile application messages, instant messages, cellular messages such as SMS, MMS, among others, as well as electronic records of any other activity, such as digital content, such as files, photographs, screenshots, browser history, internet activity, shared documents, among others.

The electronic activity can be stored on one or more data source servers. The electronic activity can be owned or managed by one or more data source providers, such as companies that utilize the services of the data processing system 9300. The electronic activity can be associated with or otherwise maintained, stored or aggregated by an electronic activity source, such as Google G Suite, Microsoft Office365, Microsoft Exchange, among others. In some embodiments, the electronic activity can be real-time (or near real-time) electronic activity, asynchronous electronic activity (such as emails, text messages, among others) or synchronous electronic activity (such as meetings, phone calls, video calls), or other activity in which two parties are communicating simultaneously.

### 1. Systems and Methods for Generating a Node Graph Using Electronic Activities

As described above, the present disclosure relates to systems and methods for constructing a node graph based on electronic activity. The node graph can include a plurality of nodes and a plurality of edges between the nodes indicating activity or relationships that are derived from a plurality of data sources that can include one or more types of electronic activities. The plurality of data sources can further include systems of record, such as customer relationship management systems, enterprise resource planning systems, document management systems, applicant tracking systems or other source of data that may maintain electronic activities, activities or records.

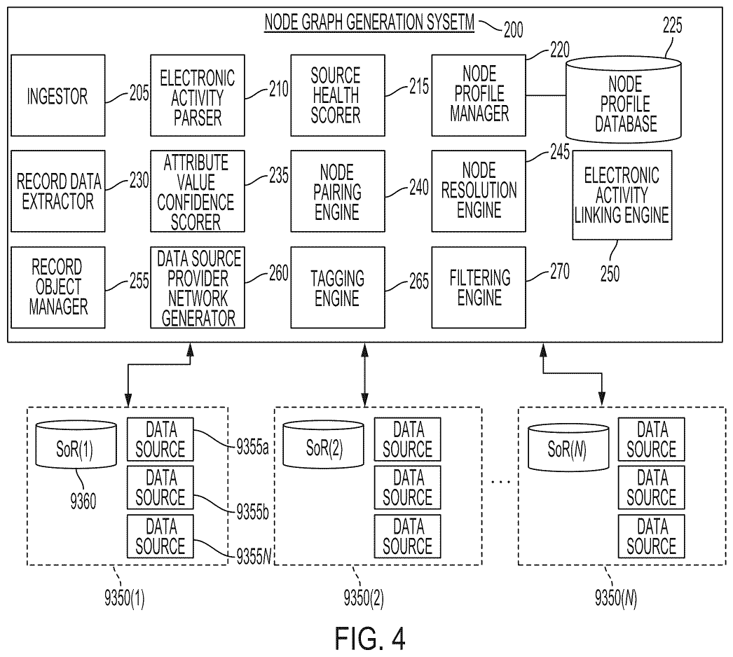

Referring now to FIG. 4, FIG. 4 illustrates a node graph generation system 200 for constructing a node graph based on electronic activity. The node graph generation system 200 can be, include or be part of the data processing system 9300 described in FIG. 3. The node graph generation system 200 can include an electronic activity ingestor 205, an electronic activity parser 210, a tagging engine 265, a source health scorer 215, a node profile manager 220, a node profile database 225, a record data extractor 230, an attribute value confidence scorer 235, a node pairing engine 240, a node resolution engine 245, an electronic activity linking engine 250, a record object manager 255, a data source provider network generator 260, a tagging engine 265 and a filtering engine 270. The node graph generation system 200 can receive electronic activity and systems of record data from one or more data source providers 9350. The data source providers can provide electronic activity or data stored or maintained on a plurality of data sources 355 and one or more systems of record 360.

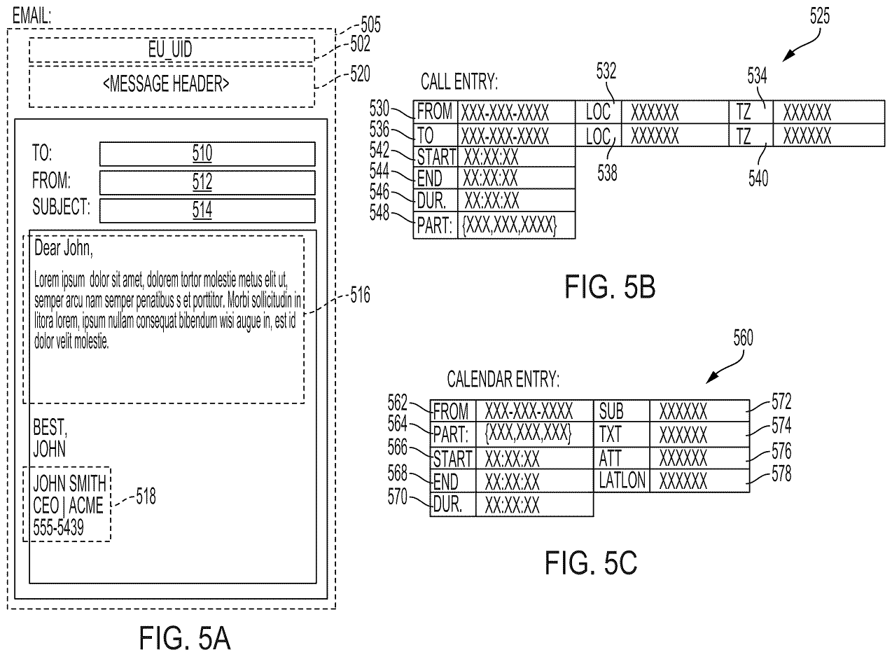

Referring now to FIG. 5A, FIG. 5A illustrates an example electronic message. The electronic message 505 can identify one or more recipients 510, one or more senders 512, a subject line 514, an email body 516, an email signature 518 and a message header 520. The message header can include additional information relating to the transmission and receipt of the email message, including a time at which the email was sent, a message identifier identifying a message, an IP address associated with the message, a location associated with the message, a time zone associated with the sender, a time at which the message was transmitted, received, and first accessed, among others. The electronic message 505 can include additional data in the electronic message 505 or in the header or metadata of the electronic message 505.

Referring now to FIG. 5B, FIG. 5B illustrates an example call entry representing a phone call or other synchronous communication is shown. The call entry 525 can identify a caller 530, a location 532 of the caller, a time zone 534 of the caller, a receiver 536, a location 538 of the receiver, a time zone 540 of the receiver, a start date and time 542, an end date and time 544, a duration 548 and a list of participants 548. In some embodiments, the times at which each participant joined and left the call can be included. Furthermore, the locations from which each of the callers called can be determined based on determining if the user called from a landline, cell phone, or voice over IP call, among others. The call entry 525 can also include fields for phone number prefixes (e.g., 800, 866, and 877), phone number extensions, and caller ID information.

Referring now to FIG. 5C, FIG. 5C illustrates an example calendar entry 560. The calendar entry 560 can identify a sender 562, a list of participants 562, a start date and time 566 location 532 of the caller, an end date and time 568, a duration 570 of the calendar entry, a subject 572 of the calendar entry, a body 574 of the calendar entry, one or more attachments 576 included in the calendar entry and a location of event, described by the calendar entry 578. The calendar entry can include additional data in the calendar entry or in the header or metadata of the calendar entry 560.

In some embodiments, the electronic activities are exchanged between or otherwise involve nodes. In some embodiments, nodes can be representative of people or companies. In some embodiments, nodes can be member nodes or group nodes. A member node may refer to a node representative of a person that is part of a company or other organizational entity. A group node may refer to a node that is representative of the company or other organizational entity and is linked to multiple member nodes. The electronic activity may be exchanged between member nodes in which case the system is configured to identify the member nodes and the one or more group nodes associated with each of the member nodes.

The data processing system 9300 or the node graph generation system 200 can be configured to assign each electronic activity a unique electronic activity identifier. This unique electronic activity identifier can be used to uniquely identify the electronic activity. Further, each electronic activity can be associated with a source that provides the electronic activity. In some embodiments, the data source can be the company or entity that authorizes the system 9300 or 200 to receive the electronic activity. In some embodiments, the source can correspond to a system of record, an electronic activity server that stores or manages electronic activity, or any other server that stores or manages electronic activity related to a company or entity. As will be described herein, the quality, health or hygiene of the source of the electronic activity may affect the role the electronic activity plays in generating the node graph. The node graph generation system 200 can be configured to determine a time at which the electronic activity occurred. In some embodiments, the time may be based on when the electronic activity was transmitted, received or recorded. As will be described herein, the time associated with the electronic activity can also affect the role the electronic activity plays in generating the node graph.

Referring again to FIG. 4, additional details relating to the functions performed by various modules of the node graph generation system are provided herein.

### A. Electronic Activity Ingestion

The electronic activity ingestor 205 can be any script, file, program, application, set of instructions, or computer-executable code that is configured to enable a computing device on which the electronic activity ingestor 205 is executed to perform one or more functions of the electronic activity ingestor 205 described herein. The electronic activity ingestor 205 can be configured to ingest electronic activities from the plurality of data source providers. The electronic activities may be received or ingested in real-time or asynchronously as electronic activities are generated, transmitted or stored by the one or more data source providers.

The node graph generation system 200 can ingest electronic activity from a plurality of different source providers. In some embodiments, the node graph generation system 200 can be configured to manage electronic activities and one or more systems of record for one or more enterprises, organizations, companies, businesses, institutions or any other group associated with a plurality of electronic activity accounts. The node graph generation system 200 can ingest electronic activities from one or more servers that hosts, processes, stores or manages electronic activities. In some embodiments, the one or more servers can be electronic mail or messaging servers. The node graph generation system 200 can ingest all or a portion of the electronic activities stored or managed by the one or more servers. In some embodiments, the node graph generation system 200 can ingest the electronic activities stored or managed by the one or more servers once or repeatedly on a periodic basis, such as daily, weekly, monthly or any other frequency.

The node graph generation system 200 can further ingest other data that may be used to generate or update node profiles of one or more nodes maintained by the node graph generation system 200. The other data may also be stored by the one or more servers that hosts, processes, stores or manages electronic activities. This data can include contact data, such as Names, addresses, phone numbers, Company information, titles, among others.

The node graph generation system 200 can further ingest data from one or more systems of record. The systems of record can be hosted, processed, stored or managed by one or more servers of the systems of record. The systems of record can be linked or otherwise associated with the one or more servers that host, process, store or manage electronic activities. In some embodiments, both the servers associated with the electronic activities and the servers maintaining the systems of record may belong to the same organization or company.

The electronic activity ingestor 205 can receive an electronic activity and can assign each electronic activity, an electronic activity unique identifier 502 to enable the node graph generation system 200 to uniquely identify each electronic activity. In some embodiments, the electronic activity unique identifier 502 can be the same identifier as a unique electronic activity identifier included in the electronic activity. In some embodiments, the unique electronic activity is included in the electronic activity by the source of the electronic activity or any other system.

The electronic activity ingestor 205 can be configured to format the electronic activity in a manner that allows the electronic activity to be parsed or processed. In some embodiments, the electronic activity ingestor 205 can identify one or more fields of the electronic activity and apply one or more normalization techniques to normalize the values included in the one or more fields. In some embodiments, the electronic activity ingestor 205 can format the values of the fields to allow content filters to apply one or more policies to identify one or more regex patterns for filtering the content, as described herein.

It should be appreciated that the electronic activity ingestor 205 can be configured to ingest electronic activities in a real-time or near real-time basis for accounts of one or more enterprises, organizations, companies, businesses, institutions or any other group associated with a plurality of electronic activity account with which the node graph generation system 200 has integrated. When an enterprise client subscribes to a service provided by the node graph generation system 200, the enterprise client provides access to electronic activities maintained by the enterprise client by going through an onboarding process. That onboarding process allows the system 200 to access electronic activities owned or maintained by the enterprise client from one or more electronic activities sources. This can include the enterprise client's mail servers, one or more systems of record, one or more phone services or servers of the enterprise client, among other sources of electronic activity. The electronic activities ingested during an onboarding process may include electronic activities that were generated in the past, perhaps many years ago, that were stored on the electronic activities' sources. In addition, in some embodiments, the system 200 can be configured to ingest and re-ingest the same electronic activities from one or more electronic activities sources on a periodic basis, including daily, weekly, monthly, or any reasonable frequency.

The electronic activity ingestor 205 can be configured to receive access to each of the electronic activities from each of these sources of electronic activity including the systems of record of the enterprise client. The electronic activity ingestor 205 can establish one or more listeners, or other mechanisms to receive electronic activities as they are received by the sources of the electronic activities enabling real-time or near real-time integration.

As more and more data is ingested and processed as described herein, the node graph generated by the node graph generation system 200 can get richer and richer with more information. The additional information, as will be described herein, can be used to populate missing fields or add new values to existing fields, reinforce field values that have low confidence scores and further increase the confidence score of field values, adjust confidence scores of certain data points, and identify patterns or make deductions based on the values of various fields of node profiles of nodes included in the graph.

As more data is ingested, the node graph generation system 200 can use existing node graph data to predict missing or ambiguous values in electronic activities such that the more node profiles and data included in the node graph, the better the predictions of the node graph generation system 200, thereby improving the processing of the ingested electronic activities and thereby improving the quality of each node profile of the node graph, which eventually will improve the quality of the overall node graph of the node graph generation system 200.

The node graph generation system 200 can be configured to periodically regenerate or recalculate the node graph. The node graph generation system 200 can do so responsive to additional data being ingested by the system 200. When new electronic activities or data is ingested by the node graph generation system 200, the system 200 can be configured to recalculate the node graph as the confidence scores (as will be described later) can change based on the information included in the new electronic activities. In some embodiments, the ingestor may re-ingest previously ingested data from the one or more electronic activity sources or simply ingest the new electronic activity not previously ingested by the system 200.

### B. Electronic Activity Parsing

The electronic activity parser 210 can be any script, file, program, application, set of instructions, or computer-executable code, which is configured to enable a computing device on which the electronic activity parser 210 is executed to perform one or more functions of the electronic activity parser 210 described herein.

The electronic activity parser 210 can be configured to parse the electronic activity to identify one or more values of fields to be used in generating node profiles of one or more nodes and associate the electronic activities between nodes for use in determining the connection and connection strength between nodes. The node profiles can include fields having name-value pairs. The electronic activity parser 210 can be configured to parse the electronic activity to identify values for as many fields of the node profiles of the nodes with which the electronic activity is associated.

The electronic activity parser 210 can be configured to first identify each of the nodes associated with the electronic activity. In some embodiments, the electronic activity parser 210 can parse the metadata of the electronic activity to identify the nodes. The metadata of the electronic activity can include a To field, a From field, a Subject field, a Body field, a signature within the body and any other information included in the electronic activity header that can be used to identify one or more values of one or more fields of any node profile of nodes associated with the electronic activity. In some embodiments, non-email electronic activity can include meetings or phone calls. The metadata of such non-email electronic activity can include a duration of the meeting or call, one or more participants of the meeting or call, a location of the meeting, locations associated with the initiator and receiver of the phone call, in addition to other information that may be extracted from the metadata of such electronic activity. In some embodiments, nodes are associated with the electronic activity if the node is a sender of the electronic activity, a recipient of the electronic activity, a participant of the electronic node, or identified in the contents of the electronic activity. The node can be identified in the contents of the electronic activity or can be inferred based on information maintained by the node graph generation system 200 and based on the connections of the node and one or more of the sender or recipients of the electronic activity.

The electronic activity parser 210 can be configured to parse the electronic activity to identify attributes, values, or characteristics of the electronic activity. In some embodiments, the electronic activity parser 210 can apply natural language processing techniques to the electronic activity to identify regex patterns, words or phrases, or other types of content that may be used for sentiment analysis, filtering, tagging, classifying, deduplication, effort estimation, and other functions performed by the data processing system 200.

In some embodiments, the electronic activity parser 210 can be configured to parse an electronic activity to identify values of fields or attributes of one or more nodes. For instance, when an electronic mail message is ingested into the node graph generation system 200, the electronic activity parser 210 can identify a FROM field of the electronic mail message. The FROM field can include a name and an email address. The name can be in the form of a first name and a last name or a last name, first name. The parser can extract the name in the FROM field and the email address in the FROM field to determine whether a node is associated with the sender of the electronic mail message.

### C. Signature Parsing

In some embodiments, the electronic activity parser 210 can be configured to identify a signature in a body of an electronic message. The parser 210 can identify the signature by utilizing a signature detection policy that includes logic for identifying patterns of signatures. In some embodiments, a signature can include one or more values of attributes, such as values for attributes including but not limited to a name, a phone number, a company name, a company division, a company address, a job title, one or more social network handles or links, an email address, among others. By parsing the signature, the electronic activity parser 210 can identify each of the values corresponding to the various fields of a node profile associated with the sender of the electronic activity. In addition to information included in the signature, the electronic activity parser can utilize information from the header of the electronic activity (i.e. first and last name) to identify where the signature is located by finding the same first name, last name and email address within a predetermined proximity or distance of each other in a region of the body, for instance, the bottom of the body. Stated in another way, the present disclosure describes methods and systems for utilizing header data of an electronic activity, which in certain embodiments, is verified to make it easier to locate a signature of an email, which may be buried under, around or with other textual content. In some embodiments, one or more of a first name, a last name and an email address extracted from the header of the electronic activity is used to identify text strings that match the extracted first name, last name and the email address. Responsive to determining that text strings matching the first name, last name and the email address are within a predetermined distance of one another, the parser 210 can identify the text strings are portions of the signature of the electronic activity. The information parsed from the signature can be used to determine a confidence score of a value of a field as further described herein with respect to the attribute value confidence scorer 235. The electronic activity parser 210 can also use signature parsing for node selection and in the identification of the node, to which the activity, containing the signature can be associated.

### D. Node Profiles

The node profile manager 220 can be any script, file, program, application, set of instructions, or computer-executable code that is configured to enable a computing device on which the node profile manager 220 is executed to perform one or more functions of the node profile manager 220 described herein. The node profile manager is configured to manage node profiles associated with each node. Node profiles of nodes are used to construct a node graph that includes nodes linked to one another based on relationships between the nodes that can be determined from electronic activities parsed and processed by the node graph generation system 200 as well as other information that may be received from one or more systems of record.

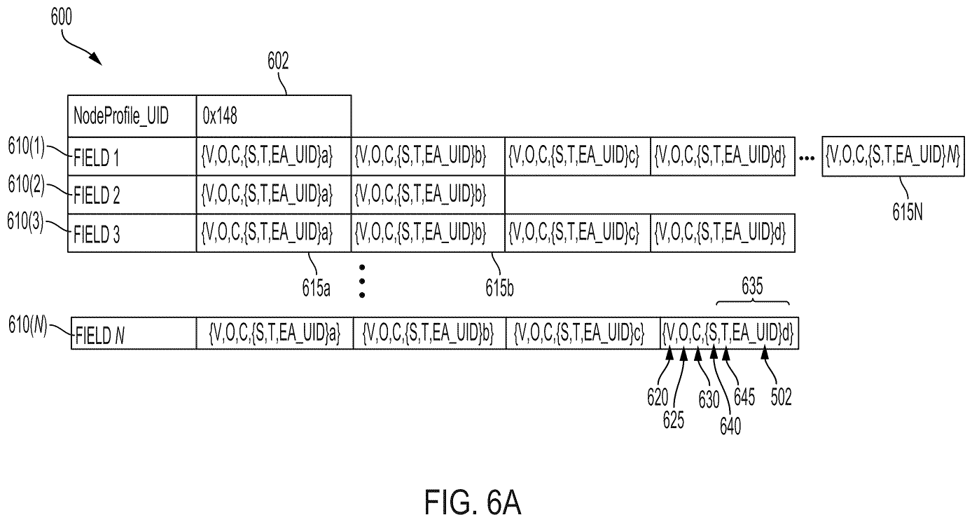

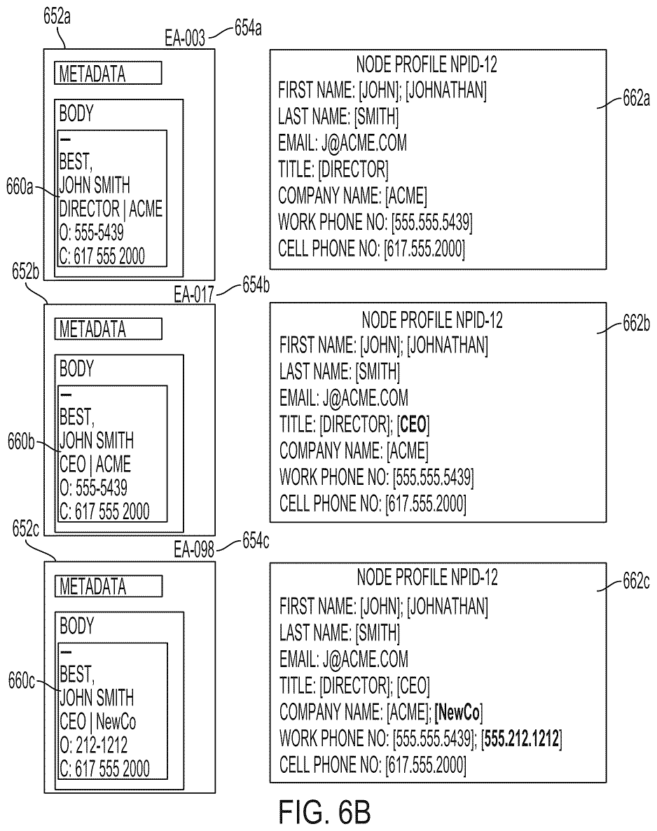

Referring now to FIG. 6A, FIG. 6A illustrates a representation of a node profile of a node. The node profile 600 can include a unique node profile identifier 602 and one or more fields or attributes 610a-610n. Each attribute 610 can include one or more value data structures 615. Each value data structure can include a value 620, an occurrence metric 625, a confidence score 630 and one or more entries 635a-n. Each entry 635 can identify a data source 640 from which the value was identified (for instance, a source of a system of record or a source of an electronic activity), a number of occurrences of the value that appear in the electronic activity, a time 645 associated with the electronic activity (for instance, at which time the electronic activity occurred) and an electronic activity unique identifier 502 identifying the electronic activity. In some embodiments, the occurrence metric 625 can identify a number of times that value is confirmed or identified from electronic activities or systems of record. The node profile manager 220 can be configured to update the occurrence metric each time the value is confirmed. In some embodiments, the electronic activity can increase the occurrence metric of a value more than once. For instance, for a field such as name, the electronic activity parser can parse multiple portions of an electronic activity. In some embodiments, parsing multiple portions of the electronic activity can provide multiple confirmations of, for example, the name associated with the electronic activity.

The node profile manager 220 can be configured to maintain a node profile for each node that includes a time series of data points for every value data structure 615 that are generated based on electronic activities identifying the respective node. The node profile manager 220 can maintain, for each field of the node profile, one or more values data structures 615. The node profile manager 220 can maintain a confidence score for each value of the field. As will be described herein the confidence score of the value can be determined using information relating to the electronic activities or systems of record that contribute to the value. The confidence score for each value can also be based on the below-described health score of the data source from which the value was received. Further, the node profile manager 220 can maintain an occurrence metric that identifies a number of times electronic activities or systems of record have contributed to the value. In some embodiments, the occurrence metric is equal to or greater than the number of electronic activities or systems of record that contribute to the value. The node profile manager 220 further maintains an array including the plurality of entries 635. As more and more electronic activities and data from more systems of record are ingested by the node graph generation system 200, values of each of the fields of node profiles of nodes will become more enriched thereby further refining the confidence score of each value.

In some embodiments, the node profile can include different types of fields for different types of nodes. Member nodes and group nodes may have some common fields but may also include different fields. Further, member nodes may include fields that get updated more frequently than group nodes. Examples of some fields of member nodes can include i) First name; ii) Last name; iii) Email; iv) job title; v) Phone; vi) Social media handle; vii) LinkedIn URL; viii) website; among others. Each of the fields can be a 3-dimensional array. In some embodiments, each field corresponds to one or more name value pairs, where each field is a name and each value for that field is a value. Examples of some fields of group nodes can include i) Company or Organization name; ii) Address of Company; iii) Phone; iv) Website; v) Social media handle; vi) LinkedIn handle; among others. Each of the fields can be a 3-dimensional array. In some embodiments, each field corresponds to one or more name value pairs, where each field is a name and each value for that field is a value.

The node profile manager 220 can maintain, for each field of each node profile, a field data structure that can be stored as a multidimensional array. The multidimensional array can include a dimension relating to data points that identify a number of electronic activities or system of records that contribute to the field or the value of the field. Another dimension can identify the source, which can have an associated trust score that can be used to determine how much weight to assign to the data point from that source. Another dimension can identify a time at which the data point was generated (for instance, in the case of a data point derived from an electronic activity such as an email, the time the data point was generated can be the time the electronic activity was sent or received). In the case of a data point being derived from a system of record, the time the data point was generated can be the time the data point can be entered into the system of record or the time the data point was last accessed, modified, confirmed, or otherwise validated in or by the system of record. These dimensions are all used to determine a confidence score of the value as will be described herein. In some embodiments, the node profile manager 220 can assign a contribution score to each data point. The contribution score can be indicative of the data point's contribution towards the confidence score of the value. The contribution score of a data point can decay over time as the data point becomes staler. The contribution scores of each of the data points derived from electronic activities and systems of record can be used to compute the confidence score of the value of a field of the node profile.

Referring now to FIG. 6B, FIG. 6B illustrates a representation of three electronic activities and a representation of three states of a node profile of a node according to embodiments of the present disclosure. As shown in FIG. 6B, three electronic activities sent at a first time, a second time and third time are shown. The first electronic activity 652a includes or is associated with a first electronic activity identifier 654a (“EA-003”). The second electronic activity 652b includes or is associated with a second electronic activity identifier 654b (“EA-017”). The third electronic activity 652c includes or is associated with a third electronic activity identifier 654b (“EA-098”). Collectively, the electronic activities can be referred to herein as electronic activities 652 or individually as electronic activity 652.

Each electronic activity can include corresponding metadata, as described above, a body, and a respective signature 660a-c included in the body of the respective electronic activity 652. As shown in FIG. 6B, each of the signatures 660a-c is different from the others.

FIG. 6B also includes three different representations of a node profile corresponding to three different times. The node profile corresponds to a node profile of the sender of the electronic activities 652 as determined by the node profile manager 220. The first representation 662a of the node profile was updated after the first electronic activity 652a was ingested by the node graph generation system 200 but before the second and third electronic activities 652b and 652c were ingested by the system 200. The second representation 662b of the node profile was updated after the first and second electronic activities 652a and 652b were ingested by the node graph generation system 200 but before the third electronic activity 652c was ingested by the system 200. The third representation 662c of the node profile was updated after all three electronic activities 652 were ingested by the node graph generation system 200.

Each of the representations 662 of the node profile can include fields and corresponding values. For example, in the first representation 662a, the field “First Name” is associated with 2 different values, John and Johnathan. The first representation 662a also includes the field “Title” which is associated with the value “Director.” In contrast, the second representation 662b and 662c both include an additional value “CEO” for the field “Title.” Furthermore, in the third representation 662c, the field “Company Name” is associated with 2 different values, Acme and NewCo in contrast with the first two representations 662a and 662b of the node profile. The values of the last name and cell phone number remain the same in all three representations 662 of the node profile.

Each of the values included in the node profile can be supported by one or more data points. Data points can be pieces of information or evidence that can be used to support the existence of values of fields of node profiles. A data point can be an electronic activity, a record object of a system of record (as will be described herein), or other information that is accessible and processable by the system 200. In some embodiments, a data point can identify an electronic activity, a record object of a system of record (as will be described herein), or other information that is accessible and processable by the system 200 that serves as a basis for supporting a value in a node profile. Each data point can be assigned its own unique identifier. Each data point can be associated with a source of the data point identifying an origin of the data point. The source of the data point can be a mail server, a system of record, among others. Each of these data points can also include a timestamp. The timestamp of a data point can identify when the data point was either generated (in the case of an electronic activity such as an email) or the record object that serves as a source of the data point was last updated (in the case when the data point is extracted from a system of record). Each data point can further be associated with a trust score of the source of the data point. The trust score of the source can be used to indicate how trustworthy or reliable the data point is. The data point can also be associated with a contribution score that can indicate how much the data point contributes towards a confidence score of the value associated with the data point. The contribution score can be based on the trust score of the source (which is based in part on a health score of the source) and a time at which the data point was generated or last updated.

A confidence score of the value can indicate a level of certainty that the value of the field is a current value of the field. The higher the confidence score, the more certain the value of the field is the current value. The confidence score can be based on the contribution scores of individual data points associated with the value. The confidence score of the value can also depend on the corresponding confidence scores of other values of the field, or the contribution scores of data points associated with other values of the field.

The table below illustrates various values for various fields and includes an array of data points that contribute to the respective value. As shown in the table, the same electronic activity can serve as different data points for different values. Further, the table illustrates a simplified form for the same of convenience and understanding.

Different values can be supported by different number of data points. The three electronic activities shown in FIG. 6B (652a-c) are included in the table below. Using the table and the representations 662a-c of the node profile, one can understand how the system 200 is capable of determining values of fields of node profiles and changes to node profiles as more electronic activities and data points are processed by the system 200.

The signature 660b is different from the signature 660a in that the title of the person John Smith has changed from Director to CEO. The data points supporting or contributing the value Director include the first electronic activity 652a but not the second electronic activity 652b. Also, the data points include information received from systems of records including data points that correspond to time periods after the value is no longer accurate. For instance, the data point DP ID225 is a data point supporting the value “Director” for the node profile even though person has been promoted to CEO. The system 200 is configured to process and accept all data points but can assign different contribution scores based on the source of the data point and allow the system 200 to accurately maintain a state of the node profile even if some of the data that is received may be inaccurate or stale. As will be described further below, it can be challenging to match electronic activities to node profiles. The system 200 can match the third electronic activity 652c to the node profile 662 even though the electronic activity identified a different email address, a different company name, and a different office number. In some embodiments, the system 200 can determine, by parsing the electronic activity, information about the sender that can be used to identify the correct node profile. In this particular case, the system 200 can rely on the first name, last name, and cell phone number (which is generally unique) to map the electronic activity to the correct node profile 662 as opposed to other node profiles including the name John Smith. Table 1:

TABLE 1

| | | | | | | Trust | Contribution |
|---|---|---|---|---|---|---|---|
| | Data Point # | DP ID | TimeStamp | Activity ID | Source | Score | Score |
| Field: First Name | Value: John [Confidence score] = 0.8 | | | | | | |
| | Data Point 1: | DP ID101 | Feb. 1, 2016 4 pm ET | EA-003 | Email | 100 | 0.6 |
| | Data Point 2: | DP ID225 | Feb. 18, 2017 2 pm ET | SOR-012 | CRM | 70 | 0.4 |
| | Data Point 3: | DP ID343 | Mar. 1, 2018 1 pm ET | EA-017 | Email | 100 | 0.7 |
| | Data Point 4: | DP ID458 | Jul. 1, 2018 3 pm ET | EA-098 | Email | 100 | 0.8 |
| | Data Point 5: | DP ID576 | Sep. 12, 2015 3 pm ET | SOR-145 | Talend | 20 | 0.2 |
| Field: First Name | Value: Johnathan [Confidence score] = 0.78 | | | | | | |
| | Data Point 1: | DP ID101 | Feb. 1, 2016 4 pm ET | EA-003 | Email | 100 | 0.6 |
| | Data Point 2: | DP ID225 | Feb. 18, 2017 2 pm ET | SOR-012 | CRM | 70 | 0.4 |
| | Data Point 3: | DP ID343 | Mar. 1, 2018 1 pm ET | EA-017 | Email | 100 | 0.7 |
| | Data Point 4: | DP ID458 | Jul. 1, 2018 3 pm ET | EA-098 | Email | 100 | 0.8 |
| | Data Point 5: | DP ID576 | Sep. 12, 2015 3 pm ET | SOR-145 | Talend | 20 | 0.2 |
| Field: Title | Value: Director [Confidence score] = 0.5 | | | | | | |
| | Data Point 1: | DP ID101 | Feb. 1, 2016 4 pm ET | EA-003 | Email | 100 | 0.6 |
| | Data Point 2: | DP ID225 | Feb. 18, 2017 2 pm ET | SOR-012 | CRM | 70 | 0.4 |
| | Data Point 3: | DP ID243 | Mar. 1, 2017 1 pm ET | EA-117 | Email | 100 | 0.65 |
| | Data Point 4: | DP ID243 | Mar. 1, 2018 1 pm ET | SOR-087 | CRM | 5 | 0.05 |
| Field: Title | Value: CEO [Confidence score] = 0.9 | | | | | | |
| | Data Point 1: | DP ID343 | Mar. 1, 2018 1 pm ET | EA-017 | Email | 100 | 0.7 |
| | Data Point 2: | DP ID458 | Jul. 1, 2018 3 pm ET | EA-098 | Email | 100 | 0.8 |
| | Data Point 3: | DP ID225 | Mar. 18, 2018 2 pm ET | SOR-015 | CRM | 65 | 0.54 |
| Field: Company | Value: Acme [Confidence score] = 0.6 | | | | | | |
| | Data Point 1: | DP ID101 | Feb. 1, 2016 4 pm ET | EA-003 | Email | 100 | 0.6 |
| | Data Point 2: | DP ID225 | Feb. 18, 2017 2 pm ET | SOR-012 | CRM | 70 | 0.4 |
| | Data Point 3: | DP ID343 | Mar. 1, 2018 1 pm ET | EA-017 | Email | 100 | 0.7 |
| Field: Company | Value: NewCo [Confidence score] = 0.9 | | | | | | |
| | Data Point 1: | DP ID458 | Jul. 1, 2018 3 pm ET | EA-098 | Email | 100 | 0.8 |
| | Data Point 2: | DP ID654 | Jul. 18, 2018 2 pm ET | EA-127 | Email | 100 | 0.85 |
| | Data Point 3: | DP ID876 | Aug. 1, 2018 1 pm ET | EA-158 | Email | 100 | 0.9 |
| Field: Cell Phone | Value: 617-555-2000 [Confidence score] = 0.95 | | | | | | |
| | Data Point 1: | DP ID101 | Feb. 1, 2016 4 pm ET | EA-003 | Email | 100 | 0.6 |
| | Data Point 2: | DP ID225 | Feb. 18, 2017 2 pm ET | SOR-012 | CRM | 70 | 0.4 |
| | Data Point 3: | DP ID343 | Mar. 1, 2018 1 pm ET | EA-017 | Email | 100 | 0.7 |
| | Data Point 4: | DP ID458 | Jul. 1, 2018 3 pm ET | EA-098 | Email | 100 | 0.8 |
| | Data Point 5: | DP ID576 | Sep. 12, 2015 3 pm ET | SOR-145 | Talend | 20 | 0.2 |
| | Data Point 6: | DP ID654 | Jul. 18, 2018 2 pm ET | EA-127 | Email | 100 | 0.85 |
| | Data Point 7: | DP ID876 | Aug. 1, 2018 1 pm ET | EA-158 | Email | 100 | 0.9 |

As a result of populating values of fields of node profiles using electronic activities, the node profile manager 220 can generate a node profile that is unobtrusively generated from electronic activities that traverse networks. In some embodiments, the node profile manager 220 can generate a node profile that is unobtrusively generated from electronic activities and systems of record.

As described herein, the present disclosure relates to methods and systems for assigning contribution scores to each data point (for example, electronic activity) that contributes to a value of a field such that the same electronic activity can assign different contribution scores to different values of a single node profile and of multiple node profiles. The contribution score can be based on a number of different electronic activities contributing to a given value of a field of a node profile, a recency of the electronic activity, among others. In some embodiments, a system of record of an enterprise accessible to the node graph generation system can include data that can also contribute to a value of a field of a node profile. The contribution score can be based on a trust score or health score of the system of record, a number of different electronic activities or systems of record contributing to the value of the field of the node profile, a number of different electronic activities or systems of record contributing to other values of the field of the node profile, a recency of the value being confirmed by the system of record, among others.

In some embodiments, a method of updating confidence scores of values of fields based on electronic activity includes associating the electronic activity to a first value of a first field, assigning a first contribution score to the first value, associating the electronic activity to a second value of a second field, assigning a second contribution score to the second value, and updating a confidence score of the first value and the second value based on the first contribution score and the second contribution score.

Furthermore, the present disclosure relates to methods and systems for maintaining trust scores for sources and adjusting a contribution score of a data point for one or more values of fields of node profiles based on the trust score of a source.

### E. Matching Electronic Activity to Node Profiles

The node profile manager 220 can be configured to manage node profiles by matching electronic activities to one or more node profiles. Responsive to the electronic activity parser 210 parsing the electronic activity to identify values corresponding to one or more fields or attributes of node profiles, the node profile manager 220 can apply an electronic activity matching policy to match electronic activities to node profiles. In some embodiments, the node profile manager 220 can identify each of the identified values corresponding to a sender of the electronic activity to match the electronic activity to a node profile corresponding to the sender.

Using an email message as an example of an electronic activity, the node profile manager 220 may first determine if the parsed values of one or more fields corresponding to the sender of the email message match corresponding values of fields. In some embodiments, the node profile manager 220 may assign different weights to different fields based on a uniqueness of values of the field. For instance, email addresses may be assigned greater weights than first names or last names or phone numbers if the phone number corresponds to a company.

In some embodiments, the node profile manager 220 can use data from the electronic activity and one or more values of fields of candidate node profiles to determine whether or not to match the electronic activity to one or more of the candidate node profiles. The node profile manager 220 can attempt to match electronic activities to one or more node profiles maintained by the node profile manager 220 based on the one or more values of the node profiles. The node profile manager 220 can identify data, such as strings or values from a given electronic activity and match the strings or values to corresponding values of the node profiles. In some embodiments, the node profile manager 220 can compute a match score between the electronic activity and a candidate node profile by comparing the strings or values of the electronic activity match corresponding values of the candidate node profile. The match score can be based on a number of fields of the node profile including a value that matches a value or string in the electronic activity. The match score can also be based on different weights applied to different fields. The weights may be based on the uniqueness of values of the field, as mentioned above. The node profile manager 220 can be configured to match the electronic activity to the node with the greatest match score. In some embodiments, the node profile manager can match the electronic activity to each candidate node that has a match score that exceeds a predetermined threshold. Further, the node profile manager 220 can maintain a match score for each electronic activity to that particular node profile, or to each value of the node profile to which the electronic activity matched. By doing so, the node profile manager 220 can use the match score to determine how much weight to assign to that particular electronic activity. Stated in another way, the better the match between the electronic activity and a node profile, the greater the influence the electronic activity can have on the values (for instance, the contribution scores of the data point on the value and as a result, in the confidence scores of the values) of the node profile. In some embodiments, the node profile manager 220 can assign a first weight to electronic activities that have a first match score and assign a second weight to electronic activities that have a second match score. The first weight may be greater than the second weight if the first match score is greater than the second match score. In some embodiments, if no nodes are found to match the electronic activity or the match score between the email message and any of the candidate node profiles is below a threshold, the node profile manager 220 can be configured to generate a new node profile to which the node profile manager assigns a unique node identifier 602. The node profile manager 220 can then populate various fields of the new node profile from the information extracted from the electronic activity parser 210 after the parser 210 parses the electronic activity.

In addition to matching the electronic activity to a sender node, the node profile manager is configured to identify each of the nodes to which the electronic activity can be matched. For instance, the electronic activity can be matched to one or more recipient nodes using a similar technique except that the node profile manager 220 is configured to look at values extracted from the TO field or any other field that can include information regarding the recipient of the node. In some embodiments, the electronic activity parser can be configured to parse a name in the salutation portion of the body of the email to identify a value of a name corresponding to a recipient node. In some embodiments, the node profile manager 220 can also match the electronic activity to both member nodes as well as the group nodes to which the member nodes are identified as members.

In some embodiments, the electronic activity parser 210 can parse the body of the electronic activity to identify additional information that can be used to populate values of one or more node profiles. The body can include one or more phone numbers, addresses, or other information that may be used to update values of fields, such as a phone number field or an address field. Further, if the contents of the electronic activity includes a name of a person different from the sender or recipient, the electronic activity parser 210 can further identify one or more node profiles matching the name to predict a relationship between the sender and/or recipient of the electronic activity and a node profile matching the name included in the body of the electronic activity.

The node profile manager 220 can be configured to maintain a node profile data structure that maintains separate values for the same field. For instance, the electronic message can be destined to john.smith@example.com <Johnathan Smith> and the body of the email states “Dear Johnathan”. The parser can be configured to identify a first name, a last name and an email address for the recipient applying logic to specific portions of the electronic activity. In certain embodiments, the node profile manager 220 can be configured to run statistical analysis of all nodes and determine that John is a very common name and thus identify that this node not only has Johnathan as first name but also John is the other First Name value. Moreover, the node profile manager 220 can be configured to determine if a value of a field is unique enough to match the electronic activity to the node based on the value of the field. If the value of the field does not meet a predetermined threshold, other values of fields may be used to match the electronic activity to a given node. In addition, values of fields may be prioritized for matching the electronic activity to the node. For instance, the name John is relatively common and as such, attempting to match an electronic activity to a node using the value “John” for the field “First Name” may be less dispositive than matching a more unique value, such as an email address. In some embodiments, the node profile manager 220 can weigh fields that have values that are relatively more unique higher than fields that have values that are relatively less unique when matching an electronic activity to a node. In some embodiments, the node profile manager 220 can be configured to restrict matching electronic activities to nodes using values of fields that are determined to not be sufficiently unique.

The node profile manager 220 can be configured to identify a node that has fields having values that match the values included in the node profile of the node. To do so, the node profile manager may determine that john.smith@example.com belongs to only one node. The node profile manager can then select that node to be the recipient of the email message. The node profile manager would then populate each of the fields of the node profile with an entry for each value of each respective field that was identified by the electronic activity parser 210. In particular, the node profile manager can generate, for each value of a field that is identified by the electronic activity parser 210, an entry in that value data structure that identifies the electronic activity, a source of the electronic activity, a time associated with the electronic activity and a number of occurrences within the electronic activity that include the value. In the email message described above, the node profile manager can update the value data structure of the Name field of the recipient node with an entry that identifies the source of the email, the time associated with the email and a total number of occurrences of the value in the email. In this case, the total number of occurrences was 2 because the first name of the recipient was listed as Johnathan and the salutation identified the name Johnathan.

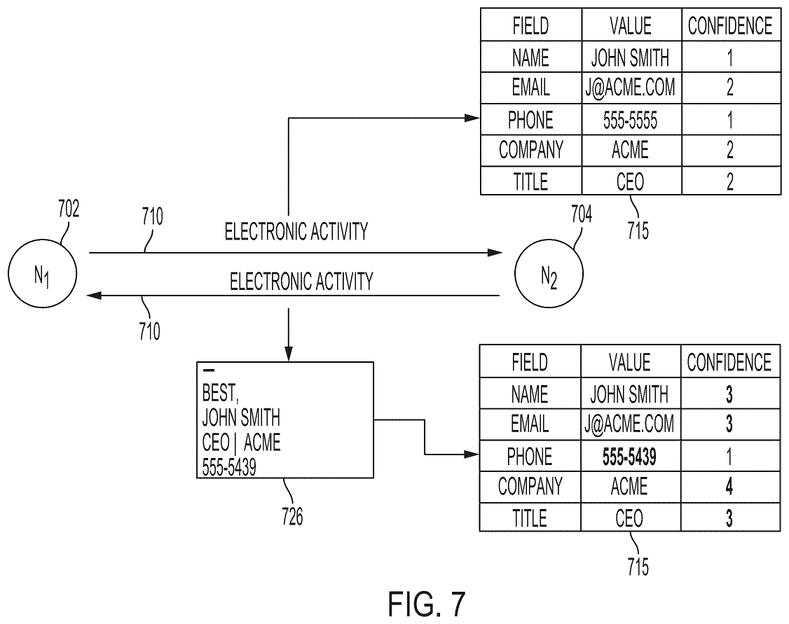

Referring briefly to FIG. 7, FIG. 7 illustrates a series of electronic activities between two nodes, N1 702 and N2 704. N1 702 may correspond to a node associated with an entity whose electronic activities are ingested by the node graph generation system 200, while node N2 704 may correspond to a node external to the entity associated with the node N1. A node profile 715 for node N2 is maintained by the node profile manager 220. Before the electronic activity 710 was ingested by the node graph generation system 200, the node profile included the five fields, name, email, phone, company and job title. This information was previously included in the node profile and may have been determined by ingesting information from a system of record. At that time, the confidence score of each of the fields is 1. When the first electronic activity is ingested by the system 200, the node profile manager can update the node profile 715 and increase the confidence score of values of fields that can be verified by the electronic activity. By virtue of the electronic activity being successfully transmitted from N1 to N2, the node profile manager 220 can update the confidence score of the email value j@acme.com and the company name Acme by parsing the email address and determining that the domain name of the email matches a domain name of the company node, to which N2 belongs. In some embodiments, the node profile manager 220 may determine that the electronic activity is successfully transmitted by determining that the N1 did not receive a bounce back electronic activity that indicates that the electronic activity was not successfully transmitted. Examples of bounce back electronic activity can include emails indicating that the destination email address is invalid or incorrect, the person is no longer with company, among others.

In some embodiments, the node graph generation system 200 can, via the electronic activity parser or through some other module, parse bounce back electronic activities to determine a reason for why the electronic activity bounced back. In some embodiments, the node graph generation system 200 can use natural language processing to determine a cause for the bounce back activity. In this way, the node graph generation system 200 can determine if an email address associated with a person or node is still valid or if it is incorrect or if the person is no longer associated with the company identified by the domain of the email address.

Node N2 can then send back a response email to node N1 that includes a signature 726 in the body of the electronic activity. The node profile manager can update, from the successful transmission of the email response and the parsing of the signature, the node profile of N2 by increasing the confidence score of the name of John Smith, the title from the signature, the company name 2 times (one of which was derived by matching the domain name of the email to the domain name of the group node in the node graph) as it is included in the email address and in the signature, and further add a new value for the phone number, which is extracted from the signature. The extracted phone number can represent his direct office number, while the phone number previously maintained in the node profile can be a general company number. In some embodiments, the system can be configured to classify phone numbers as a general company number or a direct office number based on the frequency of the number appearing in different node profiles. In some embodiments, the node graph generation system 200 can be configured to classify phone numbers as a general company number or a direct office number by performing regex patterns to determine if an “ext.” or an “x” followed by some numbers is included in the value. The regex can also be configured to identify phone number prefixes, such as “800.” The system can identify the phone numbers as the publicly known phone number of the company. In some embodiments, the node graph generation system 200 can be configured to restrict or otherwise prevent a phone number determined to be a general company number from being inserted as a value of a personal number. In some embodiments, the node graph generation system 200 can be configured to determine the value of phone numbers of other nodes corresponding to the same company and if the system determines that the number to be added to a node matches the number of multiple other nodes belonging to the same entity or company, the system can probabilistically determine, for instance, that the number is a work number and update the number as a value in the work number field (instead of a personal number field). Similar techniques can be applied for determining or inferring other information by comparing the data of a node profile to patterns observed from a plurality of related node profiles. In some embodiments, the system can determine whether the first predetermined digits (for instance, the first 6 digits) are identical to the first predetermined digits of phone numbers of other nodes belonging to the same company. If the first predetermined digits of the number match the first predetermined digits of phone numbers of other nodes belonging to the same company, the system can determine that the number is a work number. Similarly, an address extracted from a signature can be determined to be a work address if the address matches the address of other nodes belonging to the same entity or company. In this way, any value of a field of a node extracted can be determined to be specific to a company if other nodes corresponding to people belonging to the company also include the same value for the field or inter-related values in other fields. Additional details regarding increasing or adjusting the confidence score of various values of fields of node profiles based on occurrences of electronic activities are provided herein.

Generally, the node profile manager 220 can attempt to match electronic activities, such as emails, to node profiles based on an email address. However, in some instances, a user may send or receive an email address from a second email address, such as a personal email address instead of a work email address. The node profile manager 220 can analyze the electronic activity and look at other signals from the electronic activity to see if the electronic activity should be matched to a previously established node profile that corresponds to the user that does not include the second email address instead of a creating a new node profile based on the second email address.

For instance, the node profile manager 220 can be configured to identify an email that includes an email address john.smith@gmail.com. The node profile manager 220 can determine that either no node profile includes the john.smith@gmail.com as a value of an email address field or even if the email appears as a value in the email address field of a node profile, the confidence score of the value of the email address is below a certain threshold sufficient for the node profile manager 220. In some embodiments, the node profile manager 220 can apply one or more policies or rules for generating new nodes. For instance, the node profile manager 220 can implement an email address based node profile generation policy in which the system is configured to not create new node profiles if the email address corresponds to an email address of one or more predefined email systems. For instance, the email address based node profile generation policy can include one or more rules for generating new node profiles or restricting the generation of new node profiles. In some embodiments, the node profile generation policy can restrict the creation or generation of new node profiles if the email address corresponds to an email address of one or more predefined email systems. For instance, the predefined email systems can include email systems that provide “free” email addresses like “gmail.com” or “yahoo.com”. In such cases, the node profile manager 220 can be configured to use other signals from the electronic activity to attempt to match the electronic activity to a node profile for which the email address did not provide a match to a node profile. The node profile manager 220 can use fuzzy matching techniques including a first name, last name, email address prefix, a phone number or any other information that can be extracted from the email address to match the electronic activity to an existing node profile. In some embodiments, the node profile manager 220 can also identify other node profiles to which the electronic activity can be matched and identify likely node profiles based on connection strengths between the node profiles to which the electronic activity can be matched and the one or more likely node profiles.

As discussed above, in the case that John Smith inadvertently sent an email from his gmail address as opposed to his company email address, john.smith@example.com, the node profile manager 220 can use one or more of the first name, last name, phone number or other information included in the signature of the email to match the electronic activity to a node profile that includes the email address, john.smith@example.com. In this way, if other signals are pointing or expecting a work email address, the electronic activity will be matched to the node profile with the work email address.

### F. Node Profile Value Prediction and Augmentation

The node profile manager 220 can be configured to augment node profiles with additional information that can be extracted from electronic activities or systems of record or that can be inferred based on other similar electronic activities or systems of record. In some embodiments, the node profile manager 220 can determine a pattern for various fields across a group of member nodes (such as employees of the same company). For instance, the node profile manager 220 can determine, based on multiple node profiles of member nodes belonging to a group node, that employees of a given company are assigned email addresses following a given regex pattern. For instance, [first name].[last name]@[company domain].com. As such, the node profile manager 220 can be configured to predict or augment a value of a field of a node profile of an employee of a given company when only certain information of the employee is known by the node profile manager 220.

| | | Company | |
|---|---|---|---|
| First Name | Last Name | Name | Email address |
| John | Smith | Example | john.smith@example.com |
| George | Baker | Example | george.baker@example.com |
| Adam | Jones | Example | (unknown) |
| | | | adam.jones@exampl.com |
| | | | (predicted) |
| (unknown) | (unknown) | Example | linda.chan@example.com |
| Linda | Chan | | |
| (predicted) | (predicted) | | |

As shown in the table above, the node profile manager 220 can be configured to determine that the email address for Adam Jones is adam.jones@example.com by observing a regex pattern the company Example uses when assigning email addresses to its employees. In some embodiments, the node profile manager 220 can update the email address field of Adam Jones accordingly. In some embodiments, the node profile manager 220 can be configured to transmit an email to adam.jones@example.com to check whether the email address is valid or if a bounce back email is received. If no bounce back email is received indicating that the email address is not valid or cannot be found, the confidence score of adam.jones@example.com can increase even though the email address was unknown to the node graph generation system 200 based on the electronic activities ingested by the system 200.

Similarly, the node profile manager 220 can infer the first and last names of people having email addresses corresponding to a company by parsing information using the known regex patterns. As shown above, the node profile manager 220 can predict that the name of the person associated with the email address linda.chan@example.com is Linda Chan based on the regex pattern observed from other known node profiles maintained by the node profile manager 220. In some embodiments, the node profile manager 220 can infer the first and last names of people having email addresses corresponding to a company by also using other data points in the electronic activity, such as parsing email header metadata, email signature, or a greeting at the top of the email body to correlate with and confirm the name, predicted from the regex pattern above. As previously described with respect to the description associated with Table 1, the system can rely on multiple data points to match an electronic activity to a particular node profile (for instance, relying in part on the cell phone number included in the signature as discussed with respect to Table 1). In this way, further confirmation of the inference of the first name and/or last name can be obtained, thereby improving the accuracy of the node profile and the overall node graph. It should further be appreciated that if multiple people have the same name or initials, the company may assign alternate email address naming conventions for such people. For instance, a company may include a middle initial in the email address for person if the email address generated using the company's primary regex pattern for assigning email addresses is already taken. In such cases, the node profile manager 220 may again further rely on other data points in the electronic activity, such as parsing email header metadata, email signature, or a greeting at the top of the email body to infer the first and last names of the person.

In this way, by knowing the regex patterns of email addresses assigned by a company, the node profile manager 220 can be configured to predict email addresses of people at the company for which we have some information. Furthermore, if an email address is known, we can predict other information not otherwise known based on the email address. In some embodiments, even if some information is known, the confidence score of that information can be updated based on the node profile manager 220 being configured to predict certain values.

In some embodiments, the node profile manager 220 can be configured to maintain both work and personal phone numbers and work and personal geographical locations of node profiles. The node profile manager 220 can be configured to determine if a phone number extracted from an electronic activity is a work phone number or a personal phone number through one or more verification techniques. In some embodiments, the node profile manager 220 can be configured to compare the phone number of a node with phone numbers of other nodes belonging to the same company or branch/office. Corporations generally will assign phone numbers to employees that are similar to one another, for instance, all the numbers of the corporation can be 617-550-XXXX. As such, the node profile manager 220 can categorize a phone number as a work number for a node if the phone number starts with 617 550 when at least a threshold number of nodes belonging to the same email domain @example.com also have the phone number beginning with 617-550. In some embodiments, the threshold number can be 2, 3, 4, 5, or more. In some embodiments, the threshold number can be based on a percentage of another value, such as a total number of nodes belonging to the same domain and also having the phone number beginning with the same subset of digits. Conversely, the node profile manager 220 can be configured to categorize a phone number as a personal number if the phone number starts with a different set of numbers. It should be appreciated that more broadly, the node profile manager 220 can be configured to extract a regex pattern or specific template of numbers by comparing the phone numbers of multiple node profiles corresponding to the same corporation.

In some embodiments, the node profile manager 220 can be configured to compare a location of a person with an area code of a phone number associated with the person to determine if a phone number is to be classified as a work phone number or a personal phone number. If the person lives in the same area as the company's office, the person's personal phone number can have similar first few digits as the company's general phone number. In some such embodiments, the node profile manager 220 can be configured to negate the similar digits between the person's phone number and the company's assigned phone numbers to determine if the number identified in the node profile or to be included in the node profile is to be classified as a work phone number or a personal phone number. If the person lives in an area that is further away from the company based on existing information in the node profile, the node profile manager 220 can be configured to classify a number similar to the company's general phone number or having an area code corresponding to an area where the company is located as a work phone number. If the person lives in an area close to the company, the node profile manager 220 can be configured to identify the digits of the phone number that match the company's general phone number and use the remaining digits to determine if the number corresponds to a work phone number or a personal phone number of the person.

If the person lives far away from their work address, the node profile manager 220 can be configured to reduce the likelihood of assigning, as a personal phone number, a phone number that has an area code corresponding to the person's work address. More generally, the node profile manager 220 can be configured to rely on additional fields to determine if a particular number belongs to a work phone number or a personal phone number of the person.

As described herein, the node profile manager 220 can be configured to used information from node profiles to predict other values. In particular, there is significant interplay between dependent fields such as phone numbers and addresses, and titles and companies, in addition to email addresses and names, among others.

### G. Electronic Activity Tagging

The tagging engine 265 can be any script, file, program, application, set of instructions, or computer-executable code that is configured to enable a computing device on which the tagging engine 265 is executed to perform one or more functions of the tagging engine 265 described herein.

The tagging engine 265 can use information identified, generated or otherwise made available by the electronic activity parser 210. The tagging engine can be configured to assign tags to electronic activities, node profiles, systems of record, among others. By having tags assigned to electronic activities, node profiles, records ingested from one or more systems of record, among others, the node graph generation system 200 can be configured to better utilize the electronic activities to more accurately identify nodes, and determine types and strengths of connections between nodes, among others. In some embodiments, the tagging engine 265 can be configured to assign a confidence score to one or more tags assigned by the tagging engine. The tagging engine 265 can periodically update a confidence score as additional electronic activities are ingested, re-ingested and analyzed. Additional details about some of the types of tags are provided herein.

The tagging engine 265 can assign one or more tags to electronic activities. The tagging engine 265 can determine, for each electronic activity, a type of electronic activity. Types of electronic activities can include meetings, electronic messages, and phone calls. For meetings and electronic messages such as emails, the tagging engine 265 can further determine if the meeting or electronic message is internal or external and can assign an internal tag to meetings or emails identified as internal or an external tag to meetings and emails identified as external. Internal meetings or emails may be identified as internal if each of the participants or parties included in the meeting or emails belong to the same company as the sender of the email or host of the meeting. The tagging engine 265 can determine this by parsing the email addresses of the participants and determining that the domain of the email addresses map to the domain name or an array of domain names, belonging to the same company or entity. In some embodiments, the tagging engine 265 can determine if the electronic activity is internal by parsing the email addresses of the participants and determining that the domain of the email addresses map to the same company or entity after removing common (and sometimes free) mail service domains, such as gmail.com and yahoo.com, among others. The tagging engine 265 may apply some additional logic to determine if all emails belong to the same entity and use additional rules for determining if an electronic activity is determined to be internal or external. The tagging engine 265 can also identify each of the participants and determine whether a respective node profile of each of the participants is linked to the same organization. In some embodiments, the tagging engine 265 can determine if the node profiles of the participants are linked to a common group node (such as the organization's node) to determine if the electronic activity is internal. For phone calls, the tagging engine 265 may determine the parties to which the phone numbers are either assigned and determine if the parties belong to the same entity or different entities.

In some embodiments, the node graph generation system 200 can be configured to generate, maintain an update an array of domain names that belong to the same company or entity. The node graph generation system 200 may do so by monitoring electronic activities and predicting whether certain domain names belong to the same entity. The node graph generation system 200 can monitor a large number of electronic activities of an entity and determine multiple email accounts of a first domain communicate with multiple email accounts of a second domain in a manner that appears to be internal communications. In some embodiments, the node graph generation system 200 can automatically identify all possible domain names of the company based on a frequency of communications that look like internal communications between identified members of a company name, the fact that in multiple systems of record majority of the communicating node profiles belong to the same or related company profile, or by a similarity of the ending part of domain names, for example “us.ibm.com” and “uk.ibm.com”. Electronic activities can appear to be internal communications based on analyzing the words used in emails, the meeting numbers used in meeting and calendar invites, as well as determining if the email addresses match certain regex rules that may indicate that the domain names belong to the same company. For instance, electronic activities include email addresses having domain names us.example.com and uk.example.com may increase a likelihood that both us.example.com and uk.example.com appear to belong to the same company, Example. In some embodiments, if there a certain number of emails from certain users of us.example.com to other users of uk.example.com and the emails appear to be internal communications, the node graph generation system 200 or the node profile manager 220 can be configured to update the node profile of the company, Example, to include both domain names, us.example.com and uk.example.com. It should be appreciated that the node graph generation system 200 can then automatically update other node profiles and tags previously assigned to electronic activities responsive to determining that two domains belong to the same company. It should further be appreciated that the node graph generation system 200 can also automatically update confidence scores of certain values of fields of other node profiles and confidence scores of tags previously assigned to electronic activities responsive to determining that two domains belong to the same company.

In some embodiments, the tagging engine 265 can assign an internal tag or external tag to an electronic activity by applying certain logic. For instance, the tagging engine can determine that the electronic activity is internal if all the domains associated with the electronic activity are internal (or belong to the same domain). In some embodiments, if the tagging engine 265 determines that only some of the domains are internal and one or more domains are personal (i.e. not business external), then the tagging engine can be configured to attempt to match the personal email addresses to nodes and see if those nodes are linked to the same company. If the tagging engine fails to match the personal email addresses to nodes and see if those nodes are linked to the same company, the tagging engine can be configured to tag the electronic activity as external and may not link the electronic activity to a group node belonging to the domain. In some embodiments, if the tagging engine 265 determines that some domains of the email addresses included in the electronic activity are internal and some are business external, the tagging engine 265 can be configured to link the electronic activity to the group node corresponding to the external company, and further determine if individual nodes matching the email address (or first and last names) exist, and if so, linking the electronic activity with the respective individual nodes. In the event that the tagging engine 265 cannot identify an individual node that matches the email address or first and last names, the system 200 can create new individual nodes based on the respective email address or first and last names that were used to unsuccessfully identify the individual node. In the event that no individual (people) or group (company) nodes match, and the domain corresponding to the electronic activity doesn't belong to the list of free/public domains like @gmail then the system 200 can be configured to automatically create a new group (company) node or generate a flag or notification for an administrator to take an action.

The tagging engine 265 can further assign a sent tag to emails that are sent by a node associated with the data source provider from which the electronic activity was received or a received tag to emails that are received by a node associated with the data source provider from which the electronic activity was received.

In addition, the tagging engine can be configured to assign an inbound tag to received electronic activities corresponding to meeting invitations and assign an outbound tag to electronic activities corresponding to meeting invitations transmitted to other people. Moreover, meetings can be tagged with additional tags, such as a “future” tag when a meeting is scheduled for a time in the future. The “future” tag is subsequently replaced with a “past” tag once the time at which the meeting is scheduled to occur is in the past. Moreover, the tagging engine 265 can further assign tags indicating if the meeting took place or not based on other signals, such as electronic activities exchanged within a predetermined time frame of the scheduled meeting time as described herein or containing written confirmations that the meeting took place or not, such as follow-up notes between participants or cancellation notice emails. For electronic activities identified as meetings, the tagging engine 265 can further assign a tag identifying if the meeting is in person or if the meeting is a conference call. In some embodiments, the tagging engine 265 can employ a meeting type policy to determine the type of meeting. In some embodiments, the policy can include rules for parsing the location portion or body of a meeting to determine the location. If the location identifies a physical address or a room or if one of the participants included in the email is a non-human participant associated with a meeting room or other type of rooms, the tagging engine 265 can determine that the electronic activity is an in-person meeting and can assign an in-person meeting tag indicating that the meeting is an in-person meeting. In some embodiments, an in-person tag can be assigned to the electronic activity and a confidence score can be determined for the in-person tag that is assigned.

The confidence score associated with the in-person tag can be indicative of a likelihood that the meeting is actually an in-person meeting. The tagging engine 265 can further be configured to assign an occurrence tag that can be used to indicate a likelihood that the meeting occurred. The tagging engine 265 can further be configured to assign a respective participant attendance tag for each participant that attended the meeting.

To determine the confidence score associated with the in-person tag, the node graph generation system 200 can scan or analyze electronic activities associated with the participants of the meeting (and in some embodiments, the electronic activities of all users of the system 200) to identify receipts or other electronic activity, communications, among others indicative of the user physically going to the meeting. In some embodiments, the system 200 can scan electronic activities to find flight information, transportation receipts, and ride-sharing receipts, which may include information that would indicate the user physically going to the location associated with the meeting. For instance, if the meeting is at 100 Main St, San Francisco, CA on a certain date, electronic activities from an airline identifying a local airport may be used to increase the confidence score of the in-person tag. Similarly, even a flight cancellation receipt may increase the confidence score of the in-person tag. This is because even though the person may not have attended the meeting, the proof that a flight was reserved indicates that the meeting was intended to be an in-person meeting. The occurrence tag, which indicates whether the meeting actually occurred, can have its own confidence score. The greater the confidence score of the occurrence tag, the more likely the meeting occurred. As such, a flight confirmation email may increase the confidence score of the occurrence tag, while a flight cancellation email may conversely, decrease the confidence score of the occurrence tag. If multiple participants receive flight cancellation emails, the system may decrease the confidence score of the occurrence tag as it may be indicative of the meeting being canceled. However, if multiple participants received flight reservation emails and only a subset of the participants received flight cancellation emails, the system may not decrease the confidence score of the occurrence tag by the same amount as the system may assume that the meeting is still occurring but only the subset of participants are not attending. In such cases, the system may decrease the confidence score of the participant attendance tag for those participants that received flight cancellation emails. Moreover, the system can detect and parse an electronic receipt from a ride sharing service identifying one of the addresses as or near the meeting location (for example, 100 Main St, San Francisco, CA) and use the electronic activity to further increase the confidence score of the in-person meeting tag as well as the occurrence tag and the participant attendance tag.

On the other hand, the tagging engine 265 can determine that the meeting is a conference call by applying the meeting type policy and determining if a phone number or dial-in instructions are provided in the electronic activity. Furthermore, the tagging engine 265 may receive information from other engines or modules of the system to determine if participants are in close proximity to one another, based on time zone and location estimation algorithms used to predict a location of a node as well as determine or predict the locations of the participants based on electronic activities that occur within a predetermined time window of the meeting time that involve the participants. Some of the rules rely on determining a predicted work schedule of the node, a predicted location of the node, and inferred behavior before and after the meeting that can be determined from other electronic activities.

In some embodiments, the tagging engine 265 or the system 200 can be configured to cause the system 200 to initiate a call to a phone number included in a meeting invite and responsive to joining the meeting, identify one or more participants of the meeting for instance, based on identifying the phone number from which each of the participants is calling in and comparing those phone numbers to the data in the node graph or node profiles used to generate the node graph, converting speech to text, voice recognition, voice footprinting, among others. In some embodiments, the tagging engine can determine the participants who attended the meeting based on the attendees that accessed a link to a web session and in some such embodiments, used their email address to log into the web session. In some embodiments, the tagging engine 265 can determine what time a participant joined, a level of contribution of the participant during the meeting, how long the participant attended the meeting for, and generate one or more additional tags based on one or more of the participants' involvement.

As described above with respect to in-person meetings, the tagging engine 265 can also provide occurrence tags for conference call or virtual meetings as well as attendance tags for participants of such meetings. The occurrence tags can have respective confidence scores indicating the likelihood that the meeting actually occurred. Similarly, the participant attendance tags can be assigned to participants and can have respective confidence scores indicating the likelihood that the participant actually attended the meeting. The confidence scores of the occurrence tags and the attendance tags can be determined based on electronic activities that reference the meeting. In some embodiments, an electronic activity representing a phone log of a user's phone dialing into to a meeting number can be used to increase the confidence score of the occurrence tag of the meeting as well as the confidence score of the attendance tag.

The tagging engine 265 can further be configured to assign tags to people identified or included in one or more electronic activities. These tags can identify a role of the person included in the electronic activity. The tags can include a sender tag indicating a participant as a sender of the electronic activity or an organizer tag indicating a participant as an organizer of a meeting. Other similar types of tags can be assigned to participants based on whether they are included in the To line, the CC line or the BCC line. The tagging engine 265 can further be configured to tag participants based on the context of the electronic activity. For instance, if the electronic activity is determined to be associated with an opportunity, the tagging engine can assign tags to various participants, including tags indicating who the buyer is, who the seller is, who the decision maker is, who the champion is, among others. This information can be determined based on node profiles of the participants, their level of involvement in the electronic activity or the opportunity in general, among others. The tags can be assigned with certain confidence scores. As additional electronic activities are processed, the confidence scores of these tags can increase or decrease.

In some embodiments, natural language processing can be used to parse electronic activities exchanged between the participants to determine the type of meeting. For instance, an electronic activity exchanged after the meeting may indicate a phrase such as “Thanks for the lunch” which may indicate that the meeting was an in-person meeting, among others. In some embodiments, the tagging engine 265 can further tag electronic activities, such as meetings, with tags indicating if the meeting actually took place. As described above, the tagging engine 265 can tag a meeting as having taken place responsive to identifying a subsequent electronic message that included a phrase such as “Thanks for the lunch.” In some embodiments, the tagging engine can determine that the meeting is an in-person meeting by detecting an address or physical location in the body or location fields of the electronic activity. The tagging engine can further attribute a confidence score to the tag based on various data points the tagging engine relies on to determine that the electronic activity corresponds to an in-person meeting. The confidence score of the tag can increase or decrease based on additional electronic activity parsed by the system. For instance, electronic activity exchanged between the participants that may include various phrases that are detected via natural language processing, for instance, “great seeing you,” or “thanks for lunch” can increase the confidence score of the in-person tag indicating that the meeting is an in-person meeting. In addition, the electronic activity exchanged between the participants can increase the confidence score of the participant attendance tags of the sender and recipient of the email. Similarly, electronic activities including receipts of transportation (for instance, uber/lyft/flight receipts) to or from the physical location associated with the meeting may be used to increase the confidence score of the in-person tag assigned to the meeting, the occurrence tag assigned to the meeting and the participant attendance tag assigned to respective participants of the meeting. Additional details regarding tagging electronic activity are provided herein.

The tagging engine 265 can further assign tags indicating if an email is a blast email. In some embodiments, the tagging engine 265 can determine if an email is a blast email by parsing the message header of the email, identifying a message identifier field of the email and extracting the value of the message identifier field. The tagging engine can then compare the value of the message identifier field and compare the value to values of other electronic activities to determine if the values partially match. Furthermore, the tagging engine 265 can compare the words included in the body or subject line of the electronic activities that at least partially match and if the ratio of similar words to different words exceeds a threshold, the tagging engine 265 can determine if the email is a blast email. In some embodiments, the tagging engine 265 can determine electronic activities corresponding to a blast email by analyzing multiple electronic activities and identifying a subset of the multiple electronic activities as blast emails responsive to determining that each electronic activity of the subset has a low variability of word count relative to the other electronic activities in the subset and a low variability in a language complexity index relative to the other electronic activities in the subset.

In some embodiments, other signals may be used to determine if the email is a blast email, for instance, a time at which the emails were sent, and if a similar email was previously sent to a large number of people. In some embodiments, the tagging engine 265 can assign a blast email tag to an instant electronic activity responsive to determining that a similar electronic activity that is similar to the instant electronic activity above a predetermined similarity threshold was associated to a large number of nodes in a node storage database maintained by the system 200. In certain embodiments, the tagging engine 265 can learn from previously tagged electronic activities known to be blast emails and use the learnings from such electronic activities to assign a tag to an instant email having language that is similar above a predetermined similarity threshold to one or more electronic activities previously tagged as blast emails. By determining if an email is a blast email, effort estimation can be more accurately computed.

The tagging engine 265 can further assign tags indicating if an email is a cold email. In some embodiments, the tagging engine 265 can determine if an email is a cold email by applying natural language processing to identify patterns or signals that may indicate that the email is a cold email or by determining a tone of an email. In some embodiments, the tagging engine 265 may determine that an email is a cold email if the participants of the email have not exchanged any electronic activity in the past. In some embodiments, the tagging engine 265 may determine that an email sent from a sender to a recipient is a cold email if the recipient of the email has not previously transmitted a response to any electronic activity sent from the sender to the recipient in the past. In some embodiments, even if the recipient of the email has not previously transmitted a response to any electronic activity sent from the sender to the recipient in the past the tagging engine 265 may determine that an email sent from a sender to a recipient is not a cold email if the recipient and the sender have communicated via other forms of communication or via other email addresses associated with a respective node of the sender or recipient in the past. In this way, if the recipient starts a new job and gets a new email address, electronic activities sent to the new email address by a sender who has previously communicated with the recipient at the old job would not be classified or tagged as a cold emails because the node graph would indicate that the sender has communicated with the recipient in the past albeit via a different email address of the recipient that is determined based on the values of email addresses stored in a node profile of the recipient. In some embodiments, the tagging engine 265 can determine if an email is a cold email based on a number of cold emails the sender has sent in the past to one or more recipients as well as by looking at the node graph to determine a number of nodes with which the sender and recipient are commonly connected.

The tagging engine 265 can further assign tags indicating a classification of the electronic activity based on the participants included in the electronic activity. For instance, if one of the participants is a lawyer, the tagging engine 265 can assign a tag indicating that the electronic activity relates to legal. Moreover, the tagging engine 265 can further assign tags indicating a classification of the electronic activity based on the subject matter included in the electronic activity. The tagging engine 265 can determine a subject matter based on natural language processing, keywords, regex patterns or other rules that may be used to determine the subject matter. In some embodiments, filtering policies that may be provided or configured by users, companies, accounts, among others, may be used by the tagging engine 265 to assign one or more tags. Such tags can be used for filtering, matching electronic activities to record objects of systems of record, determining if emails are personal or business related, among others.

In some embodiments, the tagging engine 265 can be configured to determine if an electronic activity is a personal electronic activity or if it is a business related electronic activity. In some embodiments, the tagging engine 265 can determine that an electronic activity is personal based on parsing the contents of the electronic activity. In some embodiments, the tagging engine 265 can determine that the electronic activity is personal if the electronic activity is sent during non-work hours and the context of the electronic activity is unrelated to work. In some embodiments, the tagging engine 265 can determine that the electronic activity is personal if the participants of the electronic activity have titles or job functions that typically do not overlap or correspond to companies that do not generally engage in work related activities. In some embodiments, the tagging engine 265 can also evaluate various features, characteristics or values of fields of node profiles of the participants of the electronic activity to determine whether the electronic activity is personal. For instance, the tagging engine 265 may determine that the electronic activity is likely to be personal if the participants of the electronic activity have the same last name, as derived from the header of the electronic activity, the body or contents of the electronic activity, a signature included in the electronic activity or from the node profiles of the participants of the electronic activity. It should be appreciated that the tagging engine 265 may not need to rely on information stored in a node profile of a participant of the electronic activity to determine if the electronic activity is personal. For example, the tagging engine 265 can determine if the participants share the same last name by parsing the header of the electronic activity, the body or contents of the electronic activity, a signature included in the electronic activity. Further, if the participants have previously communicated with one another using their personal email addresses or if the contents of the electronic activity suggest that they have a prior relationship outside of work, the tagging engine 265 can determine that the participants may be related outside of work and may be configured to determine that the electronic activities exchanged between them are personal electronic activities. The tagging engine 265 can be configured to tag such electronic activities with a personal tag indicating that the electronic activity is determined to be personal. As described herein, the tagging engine 265 or the system, in general, can assign a confidence score to the tag based on how confident the system believes the electronic activity is personal (or on-work related) in nature, based on a number of methods, described above.

In some embodiments, the system 200 or the node profile manager 220 can be configured to determine that two node profiles have a personal (non-professional) relationship either based on the electronic activities exchanged between them that may be tagged with a personal tag. The system can then tag the two node profiles as having a personal relationship. The system can further determine a confidence score for the tag classifying the two node profiles based on how confident the system is in its prediction that the two node profiles have a personal relationship. In some embodiments, the system 200 or the node profile manager 220 can further determine if two nodes have a personal relationship based on commonalities in values in their node profiles, for instance, their home addresses (if they are neighbors), college or school affiliations (alumni/classmates), same last names, other non-professional affiliations, or other signals that may indicate the two node profiles may have a personal relationship.

The system 200 or the tagging engine 265 can be configured to use the personal tag between the node profiles to classify subsequent electronic activities exchanged between the node profiles. In some embodiments, as described below, the system can be configured to restrict matching electronic activities with a personal tag to record objects. The system can further be configured to either unmatch or unlink previously matched electronic activities from record objects of systems of record or remove such activities from existing data structures.

It should be appreciated that the system can conversely or similarly determine that certain electronic activities are professional in nature and tag such electronic activities with a professional tag. The system 200 can also be configured to determine that relationships between node profiles may also be professional based on their respective node profiles as well as past electronic activities exchanged between them.

It also should be appreciated that the system 200 or the tagging engine 265 can conversely or similarly determine that certain electronic activities can be more professional in nature. In some embodiments, the tagging engine 265 can determine that an electronic activity is professional if the content of the electronic activity relates to sales, recruiting, scheduling an appointment or other business related activities. The tagging engine 265 can then assign a professional tag to such an electronic activity indicating that the electronic activity is professional in nature. The tagging engine 265 can further assign a tag indicating that the electronic activity is relating to sales, recruiting or scheduling an appointment based on the context of the electronic activity. Such tags can be used to determine whether or not to match the electronic activity to a record object of a system of record. For instance, if the electronic activity relates to sales, the system 200 can tag the electronic activity with a sales tag, which the system 200 can use to determine to match the electronic activity to a record object of one or more systems of record as a sales related electronic activity can be a useful data point for a company in evaluating various aspects of their business processes. In another example, electronic activities relating to scheduling can be provided a scheduling tag, which can be used by the system 200 to filter out or restrict such electronic activities from being matched to record objects. Restricting certain electronic activities from being matched to record objects reduces the computing resources required for matching electronic activities to record objects by reducing the total volume of electronic activities to match. Restricting certain electronic activities from being matched to record objects also reduces the amount of noise in systems of record as scheduling related electronic activities add noise to the system of record.

It should be appreciated that certain tags, such as scheduling tags can be used to filter out electronic activities from a queue of electronic activities that the system 200 may attempt to match to record objects. Other such types of tags may include personal tags indicating that the electronic activity is personal, internal tags indicating that the electronic activity as internal to a company, among others.

The tagging engine 265 can further identify certain types of electronic activities that may enhance the generation of the node graph or further define roles of nodes. For instance, in an out of office email response, a person may identify a second person to contact in their absence. The tagging engine 265 can tag the electronic activity as an out of office response but further allow the node profile manager 220 to update the node profile of the nodes to indicate the potential relationship between the person who is out of office and the second person to contact in their absence or create a new node profile for that person if such a node profile doesn't yet exist.

The tagging engine 265 can assign additional tags, such as vacation tags that can be used by the node profile manager 220 to update the node profile of the node accordingly. The tagging engine 265 can assign a vacation tag to an electronic activity responsive to determining that the electronic activity corresponds to the person being on vacation. The node profile manager 220 can parse the timing of the vacation from the electronic activity and update the node profile of the person on vacation. This information can then be passed to one or more systems of record and cause the systems of record to update their settings for the given person.

In addition, the tagging engine 265 can be configured to assign a ‘no longer with company’ tag to an electronic activity responsive to parsing the electronic activity. This information can then be passed to one or more systems of record and cause the systems of record to update their settings for the given person. In addition, the ‘no longer with company’ tag can cause the system 200 to stop future emails to be sent to the person, and also trigger the system 200 to determine which company that person joined.

In some embodiments, the tagging engine 265 ca be configured to assign a ‘parental leave’ tag to an electronic activity responsive to parsing the electronic activity. The parental leave tag can be helpful to predict when a person may be returning to work. In addition, the system 200 can assign a parental leave tag to a node profile and further associate the node profile to one or more other nodes or persons that have been identified as taking over the responsibilities of the person on parental leave.

In some embodiments, the tagging engine 265 can tag an electronic activity with a deceased tag responsive to parsing the electronic activity. In some embodiments, the system 200 can then update the associated node profile indicating that the person is deceased.

In some embodiments, the tagging engine 265 can identify a unique electronic activity identifier for the electronic activity and generate a plurality of tags to assign to the electronic activity. The tagging engine 265 can generate tags to indicate if the electronic activity is external or internal, the participants associated with the electronic activity, an amount of time to generate or perform the electronic activity, job titles or seniority levels of the participants based on their job titles, departments in the organization, to which participants may belong based on their job titles, any values, opportunities or record objects with which the electronic activity may be linked or otherwise associated, one or more stages of the sales opportunity or any other system of record process, among others.

The tagging engine 265 can be configured to assign custom tags based on one or more tagging policies of one or more users or subscribers of the system 200. For instance, a subscriber of the node graph generation system 200 may desire to generate custom tags that allows the subscriber to tag all electronic activity including ride sharing receipts that identify the company's address. The subscriber may choose to then use these tags to identify all electronic activity that include ride sharing receipts that identify the company's address to gather information about the employees' use of ride sharing to and from work. The subscriber can use the information to improve business processes, such as considering providing a shuttle service to employees or negotiating with a ride sharing company for discounted pricing. The tagging engine 265 can provide a subscriber an interface through which subscribers can define policies for assigning such custom tags.

It should be appreciated that custom tags can be defined using one or more pieces of information from electronic activities. For instance, custom tags can be defined for certain email addresses, certain names, certain combination of senders and recipients, as well as based on words, phrases or other content included in the subject line or body of an electronic activity. For instance, emails that include “legal@example.com” can be tagged as Legal. Emails that mention “cell” or “mobile” and a regex pattern that matches a cell phone number in the body of an email but not part of the signature block of the email can be tagged as Cell. Emails that include a regex pattern that matches a social security number in the body of an email can be tagged as social security number, while emails that include a regex pattern that matches a credit card number in the body of an email can be tagged as credit card number. The tagging engine 265, the filtering engine 270 or the node graph generation system 200 can then use these tags to process the electronic activities tagged with these tags in accordance to one or more processing policies, such as filtering policies described herein. The filtering policies can also be customized for a given user, company or subscriber of the system 200 such that a company can deploy rules to handle such emails in accordance with the company's specific rules.

The tagging engine 265 may iteratively tag and re-tag the same electronic activities as more information is received. The tagging engine can be configured to recalculate, re-ingest and re-featurize, and re-tag all data associated with electronic activities to further refine the tags.

The tagging engine 265 can tag electronic activities based on context derived from features of such electronic activities. As described above, the tagging engine 265 can assign tags indicating a type of meeting: in-person vs. conference call; internal vs. external, a location of the participants to determine if the meeting is an in-person meeting, a time zone of the meeting, countries associated with participants of the meetings, among others.

In some embodiments, the tagging engine 265 can identify if the meeting is a conference call or a web-based meeting. In some embodiments, the type of activity can determine the types of tags to assign to the activity. For instance, for meetings, the tagging engine 265 can assign the following tags: External, internal, in-person, conference call, and custom tags, based on NLP, regex and other rules, customized by the user. For emails, the tagging engine 265 can assign the following tags: External, internal, sent, received, blast, cold. In some embodiments, blast detection techniques can be used to determine if the email is a blast email. These techniques include natural language processing analysis, blast email header analysis, volume of electronic activity for a given node, as well as MIME message data. Generally, blast emails do not include a Blast Message ID that is common across all of the blast emails. As such, detecting an email as a blast email is quite complex. In fact, blast emails are generally generated to appear as non-blast emails and as such, the present disclosure provides techniques that are based on the low variability of language complexity and word count. In some embodiments, the blast email tag assigned can include metadata identifying, for instance, the number of emails in a blast, the tool used to send the blast. The blast email tag can be used to group all emails of the blast and can include metadata about the group of emails. The tagging engine can deploy artificial intelligence to stitch the blast message ID together across multiple emails to identify if a portion of a message ID is common across multiple emails. For calls, the tagging engine 265 can assign tags to the call indicating if the call was electronically logged or manually entered. The call can be tagged based on the caller and the receiver, duration, disposition, etc.

In some embodiments, the tagging engine 265 can employ custom policies for tagging electronic activities. For instance, the tagging engine can tag every first meeting with a company as a new business meeting. The tagging engine can tag every meeting with a CXO title, such as CEO, CMO, COO, CLO, CFO, CSO, as CXO. The tagging engine can tag every meeting with CFO as finance. A reporting engine can then use these tags to generate custom reports for instance, a report identifying all new business meetings, or all activities involving finance, among others.

Tags can also be assigned for certain words, such as product names, taglines, competitor mentions, among others. By parsing emails of employees to identify the use of certain words or phrases specifically defined for a particular entity, the tagging engine can tag such electronic activities to particular products and use such electronic activities to determine if training is needed, if the correct messaging is being used or if the employees are implementing the latest messaging outlined by the company. For instance, a company can train reps to say X, but then train reps to say Y, and then use tags (from NLP) to determine which reps actually say Y. For example, if a company has 18,000 sales reps, how does the company ensure their employees are using the new training or actively selling a new product. In addition, the tagging engine 265 can apply policies to tag electronic activities based on a sentiment analysis. For instance, the tagging engine 265 can apply employee activities tags based on, negative or positive sentiment with the mention of the company's competitor or the company's feature.

In some embodiments, the tagging engine 265 can assign tags based on predicting likelihood of deal or business process completion and time to completion from electronic activities. Additional details regarding how this is determined is described herein and based in part on stage classification and the roles of the participants in the electronic activities.

In some embodiments, tags can be defined by rules. Some rules can be global rules, company rules defined by company, team level rules and user level rules.

### H. Filtering Engine

The filtering engine 270 can be any script, file, program, application, set of instructions, or computer-executable code that is configured to enable a computing device on which the filtering engine 270 is executed to perform one or more functions of the filtering engine 270 described herein.

The filtering engine 270 can use information identified, generated or otherwise made available by the tagging engine 265. The filtering engine 270 can be configured to block, remove, redact, delete, or authorize electronic activities tagged or otherwise parsed or processed by the tagging engine 265. For example, the tagging engine 265 can be configured to assign tags to electronic activities, node profiles, systems of record 9360, among others. The filtering engine 270 can be configured with a policy or rule that prevents ingestion of an electronic activity having a specific tag or any combination of tags, such as a credit card tag or social security tag. By applying filtering rules or policies to tags assigned to electronic activities, node profiles, or records from the one or more systems of record, among others, the node graph generation system 200 can be configured to block, delete, redact or authorize electronic activities at the ingestion step or redact out parts or whole values of any of the fields in the ingested electronic activities. Additional details about some of the types of filtering based on tags are provided herein.

### I. Source Health Scores Including Field-Specific Health Scores, Overall Health Scores and Determining Trust Scores Based on Health Scores

The source health scorer 215 can be any script, file, program, application, set of instructions, or computer-executable code that is configured to enable a computing device on which the source health scorer 215 is executed to perform one or more functions of the source health scorer 215 described herein. The source health scorer 215 is configured to access a system of record and retrieve all data stored in the system of record. The source health scorer 215 can then identify each record object stored in the system of record and determine, for each record object, a number of missing values of fields. The source health scorer can then generate a field-specific score for each field indicating a health or quality of each field of the system of record. The source health scorer 215 can further determine an overall health score for the source based on the field-specific scores of each field. In some such embodiments, the overall health score is based on missing field values.

The source health scorer 215 can further be configured to determine if the values of fields of record objects are accurate by comparing the values to node profiles maintained by the node profile manager 220 or to record objects maintained by the record objects manager. Based on the number of values that are inconsistent with the values maintained by the node graph generation system 200, the source health scorer can generate a health score for the system of record.

The source health scorer 215 can similarly generate a health score for each system of record. The source health scorer 215 can then compare the health score of a given system of record to the aggregate health scores of a plurality of systems of record to determine a relative trust score of the system of record. In some embodiments, the source health scorer 215 can assign different weights or scores to different types of systems of record. The source health scorer 215 may assign lower health scores to data included in a system of record that is generated using manual entry relative to node profiles that are automatically populated or generated by the node graph generation system 200 based on electronic activities.

Further, different types of sources can include emails, or email signatures within an email, one or more systems of record, among many other source types. The trust score of a source can be determined based on the health score of the source, at least in the case of a system of record. In some embodiments, the trust score assigned to electronic activity such as an email can be greater than a trust score assigned to a data point derived from a system of record as the system of record can be manually updated and changed. Additional details regarding the health score of a system of record are described below.

In some embodiments, the health score of a system of record maintained by a data source provider can be determined by comparing the record objects of the system of record with data that the system has identified as being true. For instance, the system 200 can identify, based on confidence scores of values (as described below) of fields, that certain values of fields are true. For instance, the system may determine that a value is true or correct if multiple data points provide support for the same value. In some embodiments, the multiple data points may for example, be at least 5 data points, at least 10 data points, or more. The system 200 can then, for a value of a field of a record object of the system of record, compare the value of the system of record to the value known to the system to be true. The system can repeat this for each field of a record object to determine if any values of a record object are different from the values the system knows to be true. In some embodiments, when determining the health score, the system may only compare those values of fields of record objects of the system of record that the system has a corresponding value that the system knows is true. For instance, the system may know that a phone number of a person “Roger Nadal” is 617-555-3131 and may identify such a number as true based on multiple data points. However, the system may not know an address of the person Roger Nadal. In such an instance, the system may only compare the phone number of the record object corresponding to Roger Nadal to determine the health score of the system of record but not compare the address of the person Roger Nadal as the system does not know the address of Roger Nadal. Furthermore, even if the node profile of Roger Nadal had an address but the confidence score of the address was below a predetermined threshold, the system would not compare the address from the system of record to the address of the node profile since the system does not have enough confidence or certainty that the address is true. As such, the system can be configured to determine the health score of a system of record by comparing certain values of record objects of the system of record to values the system knows as true or above a predetermined confidence score. In this way, in some embodiments, the health score of the system of record is based on an accuracy of the data included in the system of record rather than how complete the system of record is not.

As described above, the health score of a system of record can be an overall health score that can be based on aggregating individual field-specific health scores of the system of record. It should be appreciated that the system 200 can assign different weights to each of the field-specific health scores based on a volume of data corresponding to the respective field, a number of values that does not match values the system 200 knows to be true, among others.

In certain situations, the system 200 can compute trust scores for data points based on the health score of a system of record. In some embodiments, the system 200 can compute the trust score based on the overall health score of the system of record that is the source of the data point. However, in some embodiments, it may be desirable to configure the system 200 to provide more granularity when assigning a trust score to a system of record that is the source of the data point. For instance, a company may meticulously maintain phone numbers of record objects but may not be so meticulous in maintaining job titles of record objects such that the field specific health score for the phone number field of the system of record is much better than the field-specific health score for the job title field and also better than the overall health score of the system of record determined based on the aggregate of the respective field specific health scores of fields of the system of record. In some embodiments, as will be described herein, if a data point supporting a phone number of a node profile is provided by the system of record, the system 200 may be configured to determine a trust score for the data point based on the field specific health score of the field “phone number” for the system of record rather than the overall health score of the system of record, which is lower because the field specific health score of the field “job title” of the system of record is much lower than the field specific health score of the field “phone number.” By determining trust scores based on the field-specific health scores of systems of record, the system 200 may be able to more accurately rely on the data point and provide a more accurate contribution score of the data point as will be described herein. Additional concepts relating to health scores and trust scores are provided herein with respect to section 5 relating to monitoring health scores of systems of record. ix. Node Field Value Confidence Scoring

The attribute value confidence scorer 235 can be any script, file, program, application, set of instructions, or computer-executable code, that is configured to enable a computing device on which the attribute value confidence scorer 235 is executed to perform one or more functions of the attribute value confidence scorer 235 described herein. The attribute value confidence scorer 235 can be configured to determine a confidence of each value of an attribute of a node profile. The confidence of a value is determined based in part on a number of electronic activities or sources that contribute to the value, time since each electronic activity provided support or evidence of the value, time since the field value in the source system of record was last modified or confirmed by a human operator, as well as the source of the electronic activity. Electronic activity that is received from mail servers or another source that does not involve manual entry may be assigned a greater weight (or trust/health score) than a source that involves manual entry, such as a customer relationship management tool.

The attribute value confidence scorer 235 can be configured to determine a confidence of each value of an attribute of a node profile. An attribute or field can have multiple candidate values and the value with the highest confidence score can be used by the node graph generation system for confirming or validating the value of the field. The attribute value confidence scorer 235 can apply one or more scoring algorithms to determine the likelihood that each value is a correct value of the attribute. It should be appreciated that a value does not need to be current to be correct. In some embodiments, as new entities are onboarded into the system, electronic activities and systems of record corresponding to systems of record of the new entities can be processed by the system 200. In processing these electronic activities and systems of record, some electronic activities can be associated with dates many years in the past. Such electronic activities are not discarded. Rather, the system processes such electronic activities and information extracted from these electronic activities are used to populate values of fields of node profiles. Since each data point is associated with a timestamp, the data point may provide evidence for a certain value even if that value is not a current value. One example of such a value can be a job title of a person. The person many years ago may simply have been an associate at a law firm. However, that person is now a partner at the firm. If emails sent from this person's email account are processed by the system 200, more recently sent emails will have a signature of the person indicating he's a partner, while older emails will have a signature of the person indicating he's an associate. Both values, partner and associate are correct values except only partner is the current value for the job title field. A confidence score of the current value may be higher in some embodiments as data points that are more recent may be assigned a higher contribution score than data points that are older. Additional details about contribution scores and confidence scores are provided below.

In some embodiments, a node profile can correspond to or represent a person. As will be described later, such node profiles can be referred to as member node profiles. The node profile can be associated with a node profile identifier that uniquely identifies the node profile. Each node profile can include a plurality of attributes or fields, such as First name, Last name, Email, job title, Phone, LinkedIn URL, Twitter handle, among others. In some embodiments, a node profile can correspond to a company. As will be described later, such node profiles can be referred to as group node profiles. The group node profile can be similar to the member node profile of a person except that certain fields may be different, for example, a member node profile of a person may include a personal cell phone number while a group node of a company may not have a personal cell phone number but may instead have a field corresponding to parent company or child company or fields corresponding to CEO, CTO, CFO, among others. As described herein, member node profiles of people and group node profiles of companies for the most part function the same and as such, descriptions related to node profiles herein relate to both member node profiles and group node profiles. Each field or attribute can itself be a 3-dimensional array. For instance, the First name attribute can have two values: first name_1|first name_2, one Last name value and three email address values email_A|email_B|email_C. Each value can have an Occurrence (counter) value, and for each occurrence that contributes to the Occurrence value, there is an associated Source (for example, email or System of record) value and an associated timestamp (for example, today, 3;04 pm PST) value. In this way, in some embodiments, each value of a field or attribute can include a plurality of arrays, each array identifying a data point or an electronic activity, a source of the data point or electronic activity, a time associated with the data point or electronic activity, a contribution score of the data point or electronic activity and, in some embodiments, a link to a record of the data point or electronic activity. It should be appreciated that the data point can be derived from a system of record. Since systems of records can have varying levels of trust scores, the contribution score of the data point can be based on the trust score of the system of record from which the data point was derived. Stated in another way, in addition to each attribute being a 3-dimensional array, in some embodiments, each value of an attribute can be represented as a plurality of arrays. Each array can identify an electronic activity that contributed to the value of the attribute, a time associated with the electronic activity and a source associated with the electronic activity. In certain embodiments, the sub-array of occurrences, sources and times can be a fully featured sub-array of data with linkage to where the data came from.

### J. Node Profile Inferences

Certain information about a node can be inferred by the node graph generation system 200 based on information included in electronic activities ingested by the system 200. For instance, the node profile manager 220 or the electronic activity tagging engine 265 can infer if a person has left a job or switched jobs if the occurrence counter for a first value stops increasing or the frequency at which the occurrences of the first value appear has been reduced and the occurrence counter for a second value is increasing or the occurrences are more recent or are received from a source that has a higher trust score indicating that the person has changed email addresses, which can indicate that the person has switched jobs. In certain embodiments, the system 200 can determine if the second value corresponds to an email address corresponding to another employer or another company. In some embodiments, the system 200 can determine if the domain name of the email address corresponds to a list of known domain names corresponding to personal, non-work email addresses (for instance, gmail.com, outlook.com), among others. In some embodiments, the system 200 can determine if the domain name is associated with a predetermined minimum number of accounts with the same domain name. The node profile manager 220 can look at relevancy of Source, recency of time and Occurrences to determine whether to update the email field from the first email (Email_A) to the second email (Email_B).

In some embodiments, the attribute value confidence scorer 235 described herein can provide mechanisms to confirm validity of data using multiple data sources. For instance, each electronic activity can be a source of data. As more electronic activities are ingested and increase the occurrence of a value of a data field, the system can confirm the validity of the value of the field based on the number of occurrences. As such, the system described herein can compute a validity score of a value of a field of a node profile based on multiple data sources. For instance, the system can determine how many data sources indicate that the job title of the person is VP sales and can use the health score of those sources to compute a validity score or confidence score of that particular value. In addition, the timestamp associated with each electronic activity can be used to determine the validity score or confidence score of that particular value. More recent electronic activities may be given greater weight and therefore may influence the validity score of the particular value more than electronic activity that is much older.

It should be appreciated that electronic activity that is generated and ingested in real-time or near real-time can be assigned a greater weight as the electronic activity has no bias, whereas data input manually into a system of record may have some human bias. In certain embodiments in which data is imported from systems of records, the weight the data has on a confidence score of the value is based on a trust score of the system of record from which the data is imported.

In some embodiments, the attribute value confidence scorer 235 can determine a confidence score of a data point based on the data sources at any given time. A data point can be a value of a field. For example, “VP, product” can be a value for a job title of a node profile. The attribute value confidence scorer 235 can utilize the electronic activities ingested in the system to determine how many electronic activities have confirmed that the value for the job title is VP, product for that node in the email signatures present in those electronic activities. In some embodiments, the attribute value confidence scorer 235 can take into account a recency of the activity data and the source type or a health score of the source type to determine the confidence score of the value of the field. In some embodiments, the node profile manager can determine a current value of a field based on the value of the field having the highest confidence score.

### K. Stitching Time Series Together

The system can be configured to maintain a time series array for each field of a node profile that can be used to determine a timeline of events associated with the node. The system can maintain the time series array based on timestamps of all data sources of all values for each field of the node. For instance, the timeline can be used to determine a career timeline with work history information, a series of job title changes indicating promotions, among other things. In addition, the timeline of events can track a person's movement across companies or geographic locations over time as well as a list of other nodes or persons the company has been affiliated or associated with at different points in time. For instance, the job title of a node profile can include the following values over a period of time: director|vp sales|president|CEO. In certain embodiments, each of the values of the title can have an increase in a confidence score at different times and as a confidence score of a given value of the title field increases, the confidence score of the preceding value of the title field decreases.

### L. Node Connections

The node pairing engine 240 can be any script, file, program, application, set of instructions, or computer-executable code that is configured to enable a computing device on which the node pairing engine 240 is executed to perform one or more functions of the node pairing engine 240 described herein. The node pairing engine 240 can compute a connection strength between nodes based on electronic activity associated with both of the nodes. More of the recent electronic activity between the two nodes will indicate a greater connection strength. Moreover, with different tags assigned to those electronic activities, the node pairing engine 240 can further determine the relationship between the two nodes and the context in which the two nodes are connected. For instance, two nodes may be connected through their work on one or more opportunities or one node may report to the second node, among others. The context behind the relationships can be derived from the electronic activity associated with the two nodes as well as other electronic activity associated with each node independent of the other node. In certain embodiments, the node pairing engine 240 can use metadata from the electronic activities to infer connection strength or relationships. For instance, the node pairing engine can compute an average time a node takes to respond to another node and use the average time to respond to determine a connection strength. In some embodiments, the average time to respond is inversely proportional to the strength of the connection. Furthermore, the node pairing engine 240 can look at other information relating to the electronic activities to infer connection strengths. If a node responds to another node outside of business hours can be an indicator of connection strength or connection relationships.

The node pairing engine 240 can determine a connection strength between nodes at a given point in time across a timeline. As the nodes exchange further electronic activity, the connection strength can increase. The system is configured to determine the connection strength at a particular time period by filtering the electronic activities based on their respective times. In certain embodiments, the node pairing engine 240 can recalculate a connection strength between nodes responsive to a trigger. In some embodiments, the trigger can be based on a confidence score falling below a predetermined threshold indicating that the confidence in a particular value is unstable or unusable. For instance, the trigger can be satisfied or actuated when the node pairing engine 240 determines that the confidence score of a particular value of a field, such as a current employer of a person is below a predetermined confidence score (indicating that the person may no longer be at a particular company). In certain embodiments, certain changes to values in fields can trigger recalculating a connection strength irrespective of activity volume, for instance, when a new value under the employer field is added in the node.

In some embodiments, the node pairing engine 240 can determine a connection strength between two nodes by identifying each of the electronic activities that associate the nodes to one another. In contrast to other systems that may rely on whether a node has previously connected with another node, the node pairing engine 240 can determine a connection strength at various time periods based on electronic activities that occur before that time period. In particular, the node pairing engine 240 can determine staleness between nodes and take the staleness to determine a current connection strength between nodes. As such, the node pairing engine 240 can determine a temporally changing connection strength. For instance, the node pairing engine 240 can determine how many interactions recently between the two nodes. The node pairing engine 240 can determine whether the connection between the two nodes is cold or warm based on a length of time since the two nodes were involved in an electronic activity or an amount of electronic activity between the two nodes. For instance, the node pairing engine 240 can determine that the connection strength between two nodes is cold if the two nodes have not interacted for a predetermined amount of time, for instance a year. In some embodiments, the predetermined amount of time can vary based on previous electronic activity or past relationships by determining additional information from their respective node profiles. For instance, former colleagues at a company may not have a cold connection strength even if they do not communicate for more than a year.

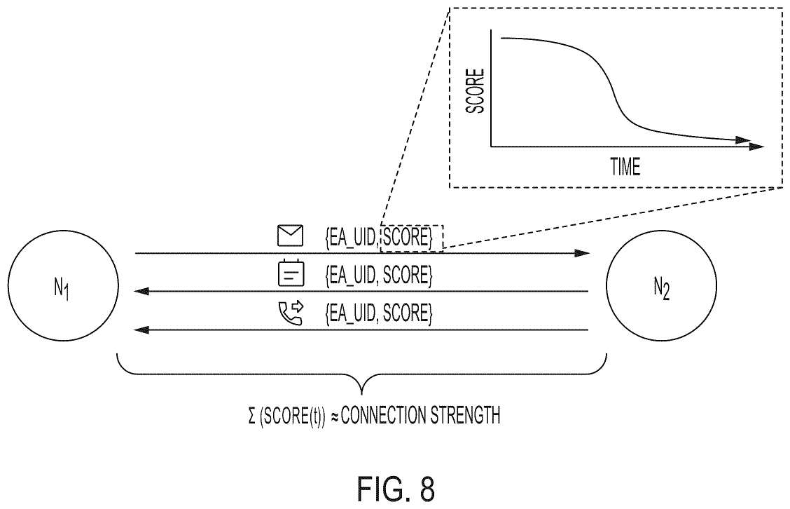

Referring briefly to FIG. 8, FIG. 8 illustrates electronic activities involving two nodes and the impact a time decaying relevancy score has on the connection strength between the two nodes. As shown in FIG. 8, N1 and N2 may exchange a series of electronic activities. The node pairing engine 240 or the system 200 can maintain a log of each of the electronic activities involving both nodes. Each electronic activity can have a unique electronic activity identifier and can identify a type of activity and maintain a time decaying relevancy score that can decrease in strength over time as time goes by. The node pairing engine 240 can compute the connection strength in part by taking the sum of the respective time decaying relevancy score of each of the electronic activities between the two nodes. In some embodiments, the node pairing engine 240 can take into account other factors for computing the connection strength, for instance, by comparing one or more fields of the node profiles. For instance, nodes that belong to the same organization, report to each other via a clear reporting logic (and lack of reporting up alternative nodes) or have previously worked together can contribute to the connection strength between the nodes.

In certain embodiments, the node pairing engine 240 can determine that a first node reports to a second node based on monitoring electronic activity exchanged between the two nodes as well as electronic activity that includes both nodes. In some embodiments, the node pairing engine 240 can apply one or more rules to predict a relationship between two nodes based on the metadata information associated with the electronic activities including both nodes.

In some embodiments, the connection strength between two nodes can be greater if the node pairing engine 240 can determine, from the electronic activities involving the two nodes, a type of relationship between the two nodes. For instance, if the node pairing engine 240 can determine that one of the nodes is the only known superior node and the other of the nodes is the likely subordinate (instead of simply knowing that the two nodes are colleagues or on the same team), the node pairing engine 240 can increase the connection strength between the two nodes.

In some embodiments, the node pairing engine 240 figured to determine the connection strength between two nodes by monitoring the type of electronic activities exchanged between them, the time of day, the day of the week, the mode of communication (email versus telephone versus text message versus office phone versus cell phone), and the duration of such communications. The system 200 can determine that if two nodes are communicating over a weekend, the connection is stronger than other connections that may only have communications limited to weekdays during office hours. The system 200 can also determine that the connection strength between two nodes may be strong if the two nodes are responding to each over the weekend, if they follow up with phone calls after receiving emails, or other patterns that may indicated a strong connection strength.

The node pairing engine 240 can be configured to identify a plurality of node pairs that have a strong connection strength. The node pairing engine 240 can then apply machine learning techniques to analyze electronic activities between the nodes of the node pair as well as analyze the node profiles of each node and the nodes to which each of the nodes are connected. The node pairing engine 240 can then generate a connection strength determination model that can be configured to determine the connection strength between two nodes using the model that is trained on node pairs known to have a strong connection strength. In some embodiments, the node pairing engine can further train the model with node pairs that have a weak connection strength in a similar fashion.

The node paring engine 240 or the tagging engine 265 can further tag the connection between the nodes as professional, personal, colleagues, ex-colleagues, alumni, classmates, among others. These tags can be updated as more and more electronic activities are processed over time and the confidence score of these tags can be adjusted accordingly. The connection strength between nodes can be used by companies to determine which employee to assign to leads, accounts, or opportunities based on the node's connections strengths with the lead, employees at the account, and employees of the account that may likely be working on the opportunity. Additional details about assigning employees to record such record objects are described below with respect to Section 12.

### M. Node Resolution

The node resolution engine 245 can be any script, file, program, application, set of instructions, or computer-executable code that is configured to enable a computing device on which the node resolution engine 245 is executed to perform one or more functions of the node resolution engine 245 described herein.

The node resolution engine 245 is configured to resolve nodes to which electronic activities are to be linked or otherwise associated. The node resolution engine 245 can use the parsed information from the electronic activity to identify values included in node profiles to determine a match score between the electronic activity and a given node profile. The node resolution engine 245 can match the electronic activity to one or more node profiles based on a match score between the electronic activity and each of the node profiles exceeding a certain threshold. Different fields are assigned different weights based on the uniqueness of each value. In some embodiments, the uniqueness of each value can be determining how many node profiles include the same value for the given field relative to the total number of node profiles.

In some embodiments, the node resolution engine 245 may match the electronic activity to the nodes between which the electronic activity occurred. The node resolution engine 245 or the node pairing engine can establish an edge between the two nodes corresponding to the electronic activity.

In some embodiments, the node resolution engine 245 may not be able to determine if the electronic activity matches any of the existing node profiles maintained by the node profile manager. In some such embodiments, the node resolution engine 245 can cause a new node profile to be generated and populated with values extracted from the electronic activity. Before the node resolution engine 245 or other module of the system 200 determines to generate a new node, the node resolution engine 245 can be configured to execute a node creation process. In some embodiments, the node resolution engine 245 can determine if the metadata of the electronic activity has attributes that are high confidence that match, such as phone number, LinkedIn ID, or email address. At the initial stage, the node resolution engine 245 can create a temporary node because not enough information is known to match the electronic activity to an existing node. As a response to the electronic activity is received, additional information can be parsed from the response to the electronic activity, which can then be used to further populate the temporary node. The temporary node can then be matched to existing node profiles to determine if an existing node matches the temporary node. If so, the temporary node can be merged with the existing node profile. In some embodiments, the process of merging involves appending the temporary node with another node because there might be mutually exclusive information that should be added.

In some embodiments, the node resolution engine 245 can perform identity resolution or deduplication based on one or more unique identifiers associated with a node profile. For instance, if one system of record provides a first email address, uniquename@example 1.com and another system of record provides a second email address, uniquename@example 2.com, while there is not a direct match, the node resolution engine 245 can resolve the two identifiers if there is a statistically significant number of matching or near matching fields, tags, or other statistical resemblances.

In particular, the node resolution engine 245 can parse the string before the @ in the email to determine one or more of a first name and last name of the person. The node resolution engine 245 can apply several techniques to do so. First, the node resolution engine 245 can check to see if there are any rules in place for the domain name of the email that indicate a particular pattern for assigning email addresses by the domain. For instance, does the company associated with the domain assign email addresses using any of the following conventions: firstname.lastname@domainname.com, FirstlnitialLastname@domainname.com, firstname@domainname.com, among others. This can be determined by looking at node profiles (and email addresses) of other people belonging to the same company. Second, the node resolution engine 245 can parse the string before the @ to attempt to recognize names from the strings. the node profile manager 220 maintains node profiles that include first names and last names and as such, the node resolution engine 245 can attempt to match a sequence of characters in the string to the list of first names and last names to see if certain names are included in the string. Upon identifying names from the string, the node resolution engine 245 can determine if the name is typically a first name or a last name based on a frequency of such names being first names or last names. Upon identifying the names with some level of statistical confidence, the node resolution engine 245 can identify a first name and a last name of a person associated with the email address and may use the first name, the last name and the company name to try and match the email address to an existing node profile of the person.

In some embodiments, the node resolution engine 245 or the node profile manager 220 can build a frequency distribution of first and last names from information included in the node profiles maintained by the node profile manager 220. The node resolution engine 245 can determine from a full name, a first name and a last name based on certain names being more common as last names and other names being more common as first names. The node resolution engine 245 can then determine a domain of the email. The node resolution engine can then calculate the probability that the string before the @ in the email corresponds to a person.

In some embodiments, the node resolution engine 245 can further determine if additional fields that could be matching—such as a social handle or a phone number to then have more surface to compare one node to other nodes to identify if any of the nodes can be merged.

In some embodiments, the node resolution engine can utilize time zone detection to resolve if two nodes belong to the same person. The system 200 can compute a time zone of each node by monitoring their electronic activities and deducing that the time zone they are in is based on the times at which the electronic activities are ingested by the system 200. For instance, the node resolution engine 245 can determine that two nodes are different if the time zones deduced from their electronic activity match different time zones.

In some embodiments, the node resolution engine 245 can be configured to periodically perform deduplication by comparing each node to every other node to determine if two nodes can be merged.

### N. Systems of Record Data Extraction

The record data extractor 230 can be any script, file, program, application, set of instructions, or computer-executable code, that is configured to enable a computing device on which the record data extractor 230 is executed to perform one or more functions of the record data extractor 230 described herein.

The record data extractor 230 can be configured to extract data from one or more records of one or more systems of record. The record data extractors 230 can identify record objects included in a system of record and extract data from each of the record objects, including values of particular fields. In some embodiments, the record data extractor 230 can be configured to extract values of fields included in the record object that are also included in the node profile maintained by the node graph generation system 200.

### O. Linking Electronic Activity to Systems of Record Data

The electronic activity linking engine 250 can be any script, file, program, application, set of instructions, or computer-executable code, that is configured to enable a computing device on which the electronic activity linking engine 250 is executed to perform one or more functions of the electronic activity linking engine 250 described herein. Additional details regarding the electronic activity linking engine is provided below.

### P. Systems of Record Record Object Management

The record object manager 255 can be any script, file, program, application, set of instructions, or computer-executable code that is configured to enable a computing device on which the record object manager 255 is executed to perform one or more functions of the record object manager 255 described herein. The record object manager 225 can be configured to maintain data regarding record objects of multiple systems of record and can be configured to augment information for a record object by extracting information from multiple record objects across a plurality of systems of record. The record object manager 255 can function as a systems of record object aggregator that is configured to aggregate data points from many systems of record, calculate the contribution score of each data point, and a timeline of the contribution score of each of those data points. The record object manager 255 or the system 200 in general can then enrich the node graph generated and maintained by the node graph generation system 200 by updating node profiles using the data points and their corresponding contribution scores. In certain embodiments, the record object manager 255 can be further configured to utilize the data from the node graph to update or fill in missing data in a target system of record provided the data in the node graph satisfies a predetermined confidence value. Additional details regarding the record object manager 255 is provided below.

### Q. Organizational Node Graph

The data source provider network generator 260 can be any script, file, program, application, set of instructions, or computer-executable code, that is configured to enable a computing device on which the data source provider network generator 260 is executed to perform one or more functions of the data source provider network generator 260 described herein. Additional details relating to the functionality of data source provider network generator 260 are provided below with respect to the generation of a company cloud described in Section 9.

### 2. Systems and Methods for Linking Electronic Activity to Systems of Record

At least one aspect of the disclosure relates to systems and methods of linking electronic activities to record objects of systems of record. The linking can be performed by the electronic activity linking engine 250 (and other components) of the node graph generation system 200 illustrated in FIG. 4.

Enterprises and other companies spend significant amount of resources to maintain and update one or more systems of records. Examples of systems of records can include customer relationship management (CRM) systems, enterprise resource planning (ERP) systems, document management systems, applicant tracking systems, among others. Typically, these systems of records are manually updated, which can result in multiple issues. First, the information that is updated into the systems of records can be incorrect either due to human error or in some cases, malicious intent. Second, the information may not be updated in a timely manner. Third, employees may not be motivated enough to even update the systems of records, resulting in systems of records that include outdated, incorrect, or incomplete information. To the extent that enterprises rely on the data included in their systems of records to make projections or predictions, such projections and predictions may also be inaccurate as the data relied upon is also inaccurate. The present disclosure aims to address these challenges that enterprises face with their existing systems of records. In particular, the present disclosure describes systems and methods for linking electronic activities to record objects included in one or more systems of record. Electronic activities, such as electronic mail, phone calls, calendar events, among others, can be used to populate, update, and maintain states of record objects of systems of record. As electronic activities are exchanged between users, these electronic activities can be parsed to not only update a node graph as described above, but further update shadow record objects for one or more systems of records of enterprises that have provided access to such systems of record to the data processing system 9300 shown in FIG. 3 or the node graph generation system 200. As described herein, the shadow record objects can be synced with the record objects of the one or more systems of records of the enterprises. In some embodiments, the electronic activities can be used to directly update the one or more systems of records of the enterprises without first updating a shadow record object. As described herein, and also referring to FIG. 3, the updating of record objects with electronic activity can refer to updating record objects within systems of record 9360 and/or shadow record objects within the shadow systems of record. By way of the present disclosure, the node graph generation system 200 can use the electronic activities to populate, maintain, and update states of record objects of systems of record.

As described herein, the node graph generation system 200 can include the electronic activity linking engine 250 that is configured to link electronic activities to record objects of one or more systems of record. By linking the electronic activities to such record objects, the electronic activity linking engine 250 can be configured to update states of one or more record objects based on the electronic activities.

Linking electronic activities to record objects can also be referred to as matching or mapping the electronic activities to record objects. Linking the electronic activities to the record objects can provide context to the electronic activities. The linked electronic activities can be stored in association with one or more record objects to which the electronic activity is linked in a system of record. Linking an electronic activity to a record object can provide context to the electronic activity by indicating what happened in the electronic activity or record object, who was involved in the electronic activity or record object, and to what contact, node, person or business process, the electronic activity or record object should be assigned. Linking the electronic activity to the record object can indirectly provide context as to why the electronic activity occurred. For example, the linking of electronic activity, such as an email, to a lead record object (in the context or a customer relationship management system) can provide context to the email that the email was sent to establish or further a lead with the intent of converting the lead into an opportunity (and the lead record object into an opportunity record object).

Although the description provided herein may refer to record objects and business processes corresponding to customer relationship management systems, it should be appreciated that the present disclosure is not intended to be limited to such systems of records but can apply to many types of systems of record including but not limited to enterprise resource planning systems, document management systems, applicant tracking systems, among others. For the sake of clarity, it should be appreciated that electronic activities can be matched to record objects directly without having to link the electronic activities to node profiles. In some embodiments, the electronic activities can be matched to node profiles and those links can be used to match some of the electronic activities to record objects.

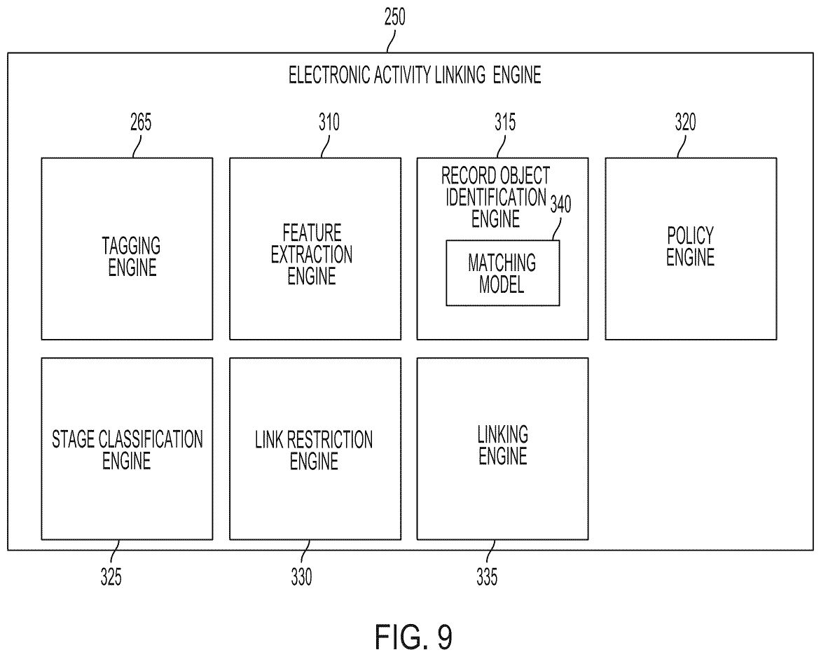

Referring now to FIG. 9, FIG. 9 illustrates a block diagram of an example electronic activity linking engine 250. The electronic activity linking engine 250 can use metadata to identify a data source provider associated with an ingested electronic activity and identify a corresponding system of record. The electronic activity linking engine 250 can match the electronic activity to a record object of the corresponding system of record. The electronic activity linking engine 250 can include, or otherwise use, a tagging engine, such as the tagging engine 265 described above to determine and apply tags to the ingested electronic activities. The electronic activity linking engine 250 can include a feature extraction engine 310 to extract features from the electronic activities that can be used to link electronic activities with one or more record objects of systems of records. In some embodiments, some of the features can include values corresponding to values stored in one or more node profiles maintained by the node graph generation system 200. The features, however, can include other information that may be used to in conjunction with information also included in node profiles to link the electronic activity to one or more record objects included in one or more systems of record.

The electronic activity linking engine 250 can include a record object identification module 315 to identify which record object or objects within a system of record to match a given electronic activity. The electronic activity linking engine 250 can include a policy engine 320. The policy engine 320 can maintain policies that include strategies for matching the electronic activities to the record objects. The electronic activity linking engine 250 can include a stage classification engine 325 to determine a shadow stage for a given opportunity record object. The electronic activity linking engine 250 can include a link restriction engine 330 that can apply one or more policies from the policy engine 320 when linking electronic activities to record objects. The linking engine 250 can link the electronic activity to the record object identified by the record object identification module 315. Additional details regarding each of the components 310-335 are further provided herein.

The features extraction engine 310 of the electronic activity linking engine 250 can be any script, file, program, application, set of instructions, or computer-executable code, that is configured to enable a computing device on which the features extraction engine 310 is executed to extract or identify features from one or more electronic activities and/or corresponding node profiles maintained by the node graph generation system 200 and use the extracted or identified features to generate corresponding feature vectors for the one or more electronic activities.

The features extraction engine 310 can be a component of the electronic activity parser 210 or otherwise interface with the electronic activity parser 210 to parse electronic activities and extract features from electronic activities. For example, the electronic activity parser 210 can parse ingested electronic activities, such as, emails, calendar meetings, and phone calls. The features extraction engine 310 can, for each electronic activity, extract various features from the electronic activity and in some embodiments, from one or more node profiles corresponding to the electronic activity, that the electronic activity linking engine 250 can use to link the electronic activity to one or more record objects of the one or more systems of record. In some embodiments, before an electronic activity can be linked to a record object of a system of record, the electronic activity can be matched to one or more node profiles in the node graph. In this way, the features extraction engine 310 can generate, based on the parsed data from the electronic activity parser 210, a feature vector for the electronic activity that can be used to link the electronic activity to a record object based on features extracted from the electronic activity as well as one or more node profiles of the node graph.

The feature vector can be an array of feature values that is associated with the electronic activity. The feature vector can include each of the features that were extracted or identified in the electronic activity by the feature extraction engine 310. For example, the feature vector for an email can include the sending email address, the receiving email address, and data parsed from the email signature. Each feature value in the array can correspond to a feature or include a feature-value pair. For example, the contact feature “John Smith” can be stored in the feature vector as “John Smith” or “name: John Smith” or “first name: John” “last name: Smith.” As described herein, the matching model 340 can use the feature vector to match or link the electronic activity to a record object. The feature vector can include information extracted from an electronic activity and also include information inferred from one or more node profiles of the node graph generation system 200. The feature vector can be used to link an electronic activity to at least particular record object of a system of record by matching the feature values of the feature vector to a record object. For instance, if the feature vector includes the values “John” for first name and “Smith” for last name, the electronic activity linking engine 250 can link the electronic activity to a record object, such as a lead record object that includes the name “John Smith” assuming other matching conditions are also met.

The features for an electronic activity can be explicit from the electronic activity. The explicit features can be determined from the metadata or content of the electronic activity. For example, the “sender's email address” of an email can be parsed from the email's header value, as described in relation to FIG. 5A. In some embodiments, some features for an electronic activity can be derived from the electronic activity. The derived features can be determined or implied based on explicit features of the electronic activity or determined from node profiles of the node graph described above. For example, an example electronic activity may not include a name of the company to which the sender belongs. In such a case, the feature extraction engine 310 can extract the name of the company to which the sender belongs from a node profile of the sender, which can include the name of the company. The name of the company can be retrieved from the node profile of the sender and saved as a value in the feature vector once retrieved from the node profile associated with the sender.

The features included in the feature vector for an electronic activity can include features associated with the generator (or sender) of the electronic activity and features associated with the recipient (or receiver) of the electronic activity. For example, sender's email address and the recipient's email address can both be used as features of the electronic activity. The features for an electronic activity can include, but are not limited to, a contact role, contact name, sender email address, recipient email address, domain, list of recipient email addresses, estimated effort, and time, features extracted from email contents using natural language processing, features extracted from email signature, time of the email sent/delivery, among others. The feature vectors can be used to match electronic activities to record objects of one or more systems of record.

The feature extractor engine 310 can further identify one or more tags assigned to an electronic activity or one or more node profiles associated with the electronic activity by the tagging engine 265 and include those tags or information relating to those tags in the feature vector. In some embodiments, these tags can be used to provide context to certain electronic activities, which can be used by the electronic activity linking engine 250 to link electronic activities to record objects of one or more systems of records.

The record object identification module 315 can be any script, file, program, application, set of instructions, or computer-executable code, that is configured to enable a computing device on which the record object identification module 315 is executed to determine or select one or more record objects to which an electronic activity should be linked or matched.

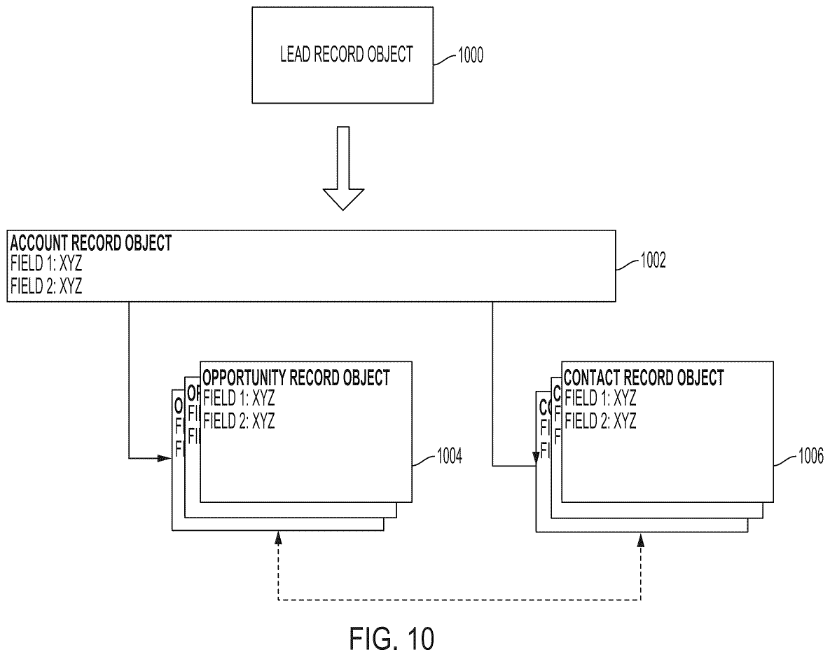

Briefly referring to FIG. 10, among others, FIG. 10 illustrates a plurality of example record objects, and their interconnections. The record objects shown in FIG. 10 can be record objects or data records of a system of record, such as a customer relationship management (CRM) system. It should be appreciated that other types of systems of records and record objects may exist and can be integrated with the node graph generation system 200. For instance, other systems of records can include Applicant Tracking Systems (ATS), such as Lever, located in San Francisco, CA or Talend by Talend Inc., located in Redwood City, CA, enterprise resource planning (ERP) systems, customer success systems, such as Gainsight located in Redwood City, CA, Document Management Systems, among others.

The systems of record can be one or more of shadow systems of record of the data processing system 9300 or the systems of record of the data source providers. Additional details relating to the shadow systems of record of the data processing system 9300 are provided below. As illustrated in FIG. 10, the record objects can include a lead record object 1000, an account record object 1002, an opportunity record object 1004, or a contact record object 1006. Each of the different types of record objects can generally be referred to as record objects.

Each record object can be a data structure or data file into which data is stored or associated. The lead record object 1000 can be a low quality object that includes unqualified contact information typically received through a web inquiry. A lead record object can correspond to one or more stages. Upon reaching a final “Converted” stage, a lead record object can be converted in a one-to-many relationship into a Contact record object (person), an Account record object (company, if new, or added to existing account) and an Opportunity record object (if there is an opportunity for a deal here or added as contact role into existing opportunity).

For example, the lead record object 1000 can include the contact information for a lead or prospective buyer. The lead record object 1000 can include fields, such as, Address, City, Company, CompanyDunsNumber, Description, Email, Industry, NumberOfEmployees, Phone, job title, and Website, among others.

The account record object 1002 can be a data structure that includes fields associated with an account that is held with the data source provider. The fields can include AccountNumber, BillingAddress, Description, Industry, Fax, DunsNumber, LastActivityDate, MasterRecordId, Name, NumberOfEmployees, Ownership, Website, YearStarted, and IsPersonAccount, among others. A system of record can include an account record object 1002 for each of the data provider's customers.

The system of record can include multiple account record objects 1002 for a given customer. For example, the system of record can include an account record object 1002 for each division of a given customer. The account record object 1002 can be stored with one or more opportunity record objects 1004.

In some embodiments, the CRM can include partner record objects, which can also be referred to as partner account record objects. A partner account record object can be similar to an account record object. The partner account record object can include an additional field to designate the record object as a partner account record object rather than a standard account record object. The partner account record object can be an account record object that is associated with a partner to the data source provider. For example, the partner account record object can be an account record object for a distributor of the data source provider that distributes goods to the company of the account record object.

The opportunity record objects 1004 can be data structures that include a plurality of fields for a given opportunity. The opportunity can indicate a possible or planned deal with a customer for which an account record object is already stored in the system of record. The opportunity record objects 1004 can include fields such as AccountId, Amount, CampaignId, CloseDate, Description, ExpectedRevenue, Fiscal, HasOpenActivity, IsClosed, IsWon, LastActivityDate, Name, Ownerld, StageName, Territory2Id, and Type, among others. One or more contact record objects 1006 can be associated with the account record object 1002. The contact record objects 1006 can be data structures that include fields associated with a contact. The contact record object 1006 can include fields such as AccountId, AssistantName, Birthdate, Department, Description, DoNotCall, Email, Fax, FirstName, HasOptedOutOfEmail, HomePhone, LastName, MailingAddress, and MobilePhone, among others.

One or more contact record objects 1006 can be associated with an opportunity record object 1004 via an Opportunity Contact Role object (OCR). For example, a lead to sell a service to a potential customer can convert into an opportunity record object 1004 when the customer begins the negotiation process to purchase the service. A contact record object 1006 can be generated for each of the customer's employees involved in the purchase. Each of the contact record objects 1006 can be associated with the opportunity record object 1004 for the sale via Opportunity Contact Roles, which contain their own metadata about involvement of specific individuals in the opportunity, such as their Role in this particular opportunity or whether they are the Primary Contact of the Account in this Opportunity.

In some embodiments, a lead record object 1000 can be converted into a contact record object 1006, an account record object 1002, and an opportunity record object 1004. For example, a lead record object 1000 can be converted into a new contact record object 1006, account record object 1002, and opportunity record object 1004 once the lead record object 1000 after a predetermined number and nature of electronic activities are associated with the lead record object 1000. Continuing this example, the lead record object 1000 can be generated based on a web inquiry from an interested party (lead) or via a cold email being sent to a potential new customer. If the customer responds and passes qualification criteria, the lead record object 1000 can be converted into a new contact record object 1006, account record object 1002, and opportunity record object 1004. In some embodiments, the lead record object 1000 can be converted into a, for example, contact record object 1006 that can get attached to or linked with an existing account record object 1002 and an existing opportunity record via an Opportunity Contact Role record.

The fields of each of the different record object types can include hierarchical data or the fields can be linked together in a hierarchical fashion. The hierarchical linking of the fields can be based on the explicit or implicit linking of record objects. For example, a contact record object 1006 can include a “Reports To” field into which an identifier of the contact can be stored. The “Reports To” field can indicate an explicit link in a hierarchy between two contact record objects 1006 (e.g., the first contact record object 1006 to the contact record object 1006 of the person identified by the “Reports To” field). In another example, the linking of the record objects can be implicit and learned by the electronic activity linking engine 250. For example, the electronic activity linking engine 250 can learn if multiple customers have the same value for a “Parent Account” field across multiple system of record sources with high trust score and derive a statistically significant probability that a specific account belongs to (e.g., is beneath the record object in the given hierarchy) another account record object.

Referring to FIG. 9, among others, the record object identification module 315 can determine, for a given electronic activity to which record object the electronic activity should be linked. Linking the electronic activity to one or more record objects can enable the status, metrics, and stage of the deal or opportunity to be tracked and analyzed, or the context in which the electronic activity was performed to be understood programmatically. Linking electronic activities to the record objects can also enable employee performance to be measured as described herein. The record object identification module 315 can identify a record object of one of the data processing system's shadow systems of record using the feature vectors and node graph. In this way, the record object identification module 315 can assist, aid or allow the electronic activity linking engine 250 to match the electronic activity with a record object using one or more matching models 340.

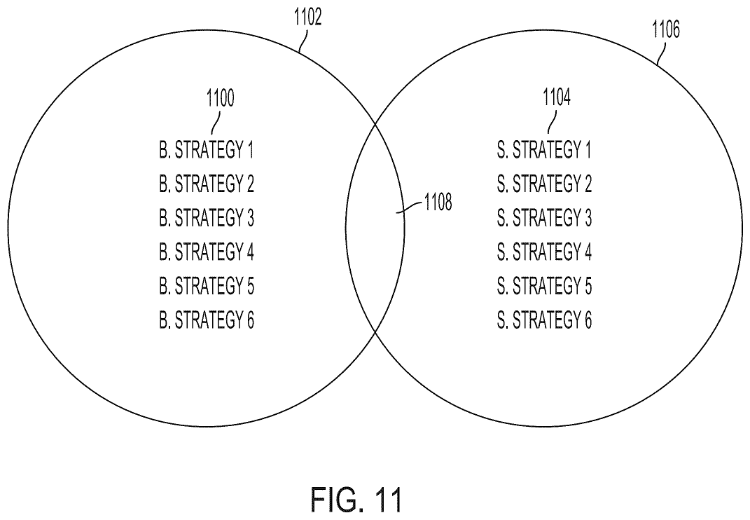

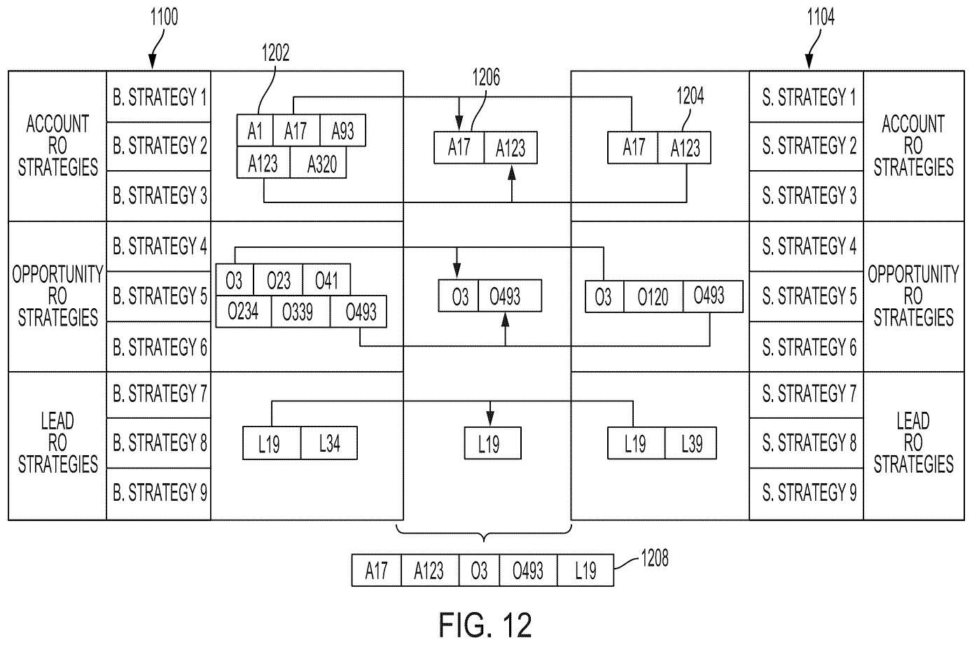

The record object identification engine 315 can include one or more matching models 340. A matching model 340 can be trained or programmed to aid in matching electronic activities to record objects to allow the electronic activity linking engine 250 to link the electronic activities to the matched record objects. For example, the record object identification engine 315 can include or use one or more matching models 340 to assist, aid or allow the electronic activity linking engine 250 to match electronic activities to record objects. In some embodiments, each of the one or more matching models 340 can be specific to a particular data source provider, electronic activity type, or record object type. In some embodiments, the record object identification engine 315 can include a single matching model that the record object identification engine 315 can use to match electronic activities ingested by the data processing system 9300 to any number of a plurality of record objects of a plurality of systems of records. In some embodiments, the matching models 340 can be data structures that include rules or heuristics for linking electronic activities with record objects. The matching models 340 can include matching rules (which can be referred to as matching strategies) and can include restricting rules (which can be referred to as restricting strategies or pruning strategies). As described further in relation to FIGS. 11 and 12, the record object identification engine 315 can use the matching strategies to select candidate record objects to which the electronic activity could be linked and use the restricting strategies to refine, discard, or select from the candidate record objects. In some embodiments, the matching models 340 can include a data structure that includes the coefficients for a machine learning model for use in linking electronic activities with record objects.

In some embodiments, the matching model 340 used to link electronic activities to one or more record objects can be trained using machine learning or include a plurality of heuristics. For example, as described above the features extraction engine 310 can generate a feature vector for each electronic activity. The matching model 340 can use neural networks, nearest neighbor classification, or other modeling approaches to classify the electronic activity based on the feature vector. In some embodiments, the record object identification engine 315 can use only a subset of an electronic activity's features to match the electronic activity to a record object.

In some embodiments, the record object identification engine 315 can use matching models 340 trained with machine learning to match, for example, the electronic activity to a record object based on a similarity of the text in and the sender of the electronic activity with the text in and sender of an electronic activity previously matched to a given electronic activity. In some embodiments, the matching model 340 can be updated as electronic activities are matched to record objects. For example, a matching model 340 can include one or more rules to use when matching an electronic activity to a record object. If a user matches an electronic activity to a record object other than the record object to which the electronic activity linking engine 250 matched the electronic activity, record object identification engine 315 can update the matching model 340 to alter or remove the rule that led to the incorrect matching.

In some embodiments, once an electronic activity is matched with a record object, a user can accept or reject the linking. Additionally, the user can change or remap the linking between the electronic activity and the record object. An indication of the acceptance, rejection, or remapping can be used to update the machine learning model or reorder the matching strategies as discussed in relation to FIGS. 11 and 12. The updated model can be used in the future linking of electronic activity to nodes and the nodes to record objects by the record object identification engine 315. To train the machine learning models, the system can scan one or more systems of record that include manually matched electronic activity and record objects. The previous manually matched data can be used as a training set for the machine learning models.

In some embodiments, the matching model 340 can include a plurality of heuristics with which the record object identification engine 315 can use to link an electronic activity to one or more record objects. The heuristics can include a plurality of matching algorithms that are encapsulated into matching strategies. The record object identification engine 315 can apply one or more matching strategies from the matching models 340 to the electronic activity to select which record object (or record objects) to link with the electronic activity. In some embodiments, the record object identification engine 315 can use the matching strategies to select candidate record objects to which the electronic activity can be linked. The record object identification engine 315 can use a second set of strategies (e.g., restricting strategies) to prune the candidate record objects and select to which of the candidate record objects the electronic activity should be linked.

The application of each strategy to an electronic activity can result in the selection of one or more record objects (e.g., candidate record objects). The selection of which matching strategies to apply to an electronic activity can be performed by the policy engine 320. The policy engine 320 is described further below, but briefly, the policy engine 320 can generate, manage or provide a matching policy for each of the data source providers 9350. The policy engine 320 can generate the matching policy automatically. The policy engine 320 can generate the matching policy with input or feedback from the data source provider 9350 to which the matching policy is associated. For example, the data source provider (for example, an administrator at the data source provider) can provide feedback when an electronic activity is incorrectly linked and the matching policy can be updated based on the feedback.

A given matching policy can include a plurality of matching strategies and the order in which the matching strategies should be applied to identify one or more record objects to which to link the electronic activity. The record object identification module 315 can apply one or more of the plurality of matching strategies from the matching models 340, in a predetermined order specified or determined via the matching policy, to identify one or more candidate record objects. The record object identification module 315 can also determine, for each matching strategy used to identify a candidate record object, a respective weight that the record object identification module 315 should use to determine whether or not the candidate record object is a good match to the electronic activity. The record object identification module 315 can be configured to compute a matching score for each candidate record object based on the plurality of respective weights corresponding to the matching strategies that were used to identify the candidate record object. The matching score can indicate how closely a record object matches the electronic activity based on the one or more matching strategies used by the record object identification module 315.

One or more of the matching strategies can be used to identify one or more candidate record objects to which the electronic activity linking engine can match a given electronic activity based on one or more features (e.g., an email address) extracted from the electronic activity or tags assigned to the electronic activity. In some embodiments, the features can be tags assigned by the tagging engine 265. In some embodiments, the electronic activity can be matched to a node profile that is already matched to a record object, thereby allowing the record object identification module 315 to match the electronic activity to a record object previously matched or linked to a node profile with which the electronic activity may be linked. In addition, the matching strategies can be designed or created to identify candidate record objects using other types of data included in the node graph generation system, or one or more systems of record, among others. In some embodiments, the matching strategies can be generated by analyzing how one or more electronic activities are matched to one or more record objects, including using machine learning techniques to generate matching strategies in a supervised or unsupervised learning environments.

Subsequent strategies can be applied to prune or restrict the record objects that are selected as potential matches (e.g., candidate record objects). For example, and also referring to FIG. 11, FIG. 11 illustrates the restriction of a first grouping 1102 of record objects with a second grouping 1106 of record objects. A first plurality of strategies 1100 can be applied to select a first grouping 1102 of record objects. A second plurality of strategies 1104 can be applied to identify a second grouping 1106 of record objects that can be used to restrict or prune the first grouping 1102 of record objects. For example, the record object identification module 315 can select the record object to which the electronic activity is linked from the overlap 1108 of the groupings 1102 and 1106.

For example, and also referring to FIG. 12, among others, FIG. 12 illustrates the application of a first plurality of matching strategies and a second plurality of matching strategies to generate one or more grouping of record objects and then selecting record objects that satisfy both the first plurality of matching strategies and the second plurality of matching strategies. In some embodiments, the first plurality of matching strategies can be configured to generate the first grouping 1102 of record objects shown in FIG. 11, while the second plurality of matching strategies 1204 can be configured to generate the second grouping 1104 of record objects. In some embodiments, the first plurality of matching strategies 1100 can be associated with one or more recipients of the electronic activity to be matched and the second plurality of matching strategies 1104 can be associated with a sender of the electronic activity to be matched. The candidate record objects selected by the first plurality of matching strategies 1100 and the second plurality of matching strategies 1104 can be filtered, pruned or otherwise discarded from being matched with the electronic activity using restricting strategies (described further below). In some embodiments, the first plurality of strategies can be referred to as buyer-side or recipient-side strategies and the second plurality of strategies can be referred to as seller-side or sender-side strategies. The policy engine 320 can select one or more matching strategies of the first plurality of matching strategies 1100, second plurality of matching strategies 1104 and restricting strategies for the record object identification engine 315 to apply in a predetermined order. The matching strategies of the first plurality of matching strategies 1100 and the second plurality of matching strategies 1104 can each be configured to select one of the types of record objects. For example, the matching strategies 1100 and 1104 can each be configured to select one of a lead record object 1000, an account record object 1002, an opportunity record object 1004, a partner record object, among others. For example, a matching strategy can be used to match an electronic activity to an account record object 1002 in the shadow system of records based on an email address extracted from the electronic activity via a number of sequentially used matching strategies. The restriction strategies can be used to remove one or more record objects that are selected by any of the first plurality of matching strategies 1100 or any of the second plurality of matching strategies 1104.

In an example where the electronic activity includes the email “john.smith@example.com,” the record object identification module 315 can use a first matching strategy, such as a matching strategy for selecting the account record object based on email addresses to identify one or more candidate record objects that may match the email address field of the electronic activity. First, the record object identification module 315 can return all contact record objects with “john.smith@example.com” in the email field. The record object identification engine 315 can then identify the account record objects that are linked with each of the contact record objects with “john.smith@example.com” in the email field.

In some embodiments, if the system was not able to find a contact record object with the field (or other fields) containing “john.smith@example.com”, the system can use a secondary matching strategy 1100 and find an account record object with the domain name that matches the domain name of the email “@example.com”. If after applying the restricting strategies and eliminating possible options, only one account with such domain name is left, the system would have identified the account to which potential contact with email address “john.smith@example.com” should belong and the original electronic activity should be linked to. In this case, the system could automatically create a contact record with email “john.smith@example.com”, linked to the account record with domain name “example.com” and then associate electronic activity from which this process started to the newly created contact record object and right account record object. It is worth noting that the order in which matching strategies 1100 and 1104 and the restriction strategies are applied can impact and modify outcomes of matching model 340.

Still referring to FIG. 12, the record object identification engine 315 can use one or more of the matching strategies 1100 associated with account record objects to generate a matched candidate record object array 1202 that identifies one or more candidate record objects that were identified based on the matching strategies 1100 associated with account record objects. The record object identification engine 315 can generate three matched record object arrays 1202. Each of the matched record object arrays can be associated with a different one of the record object types. For example, the record object identification engine 315 can generate an account record object array, an opportunity object array, a contact object array, a lead object array, and a partner object array (not shown). The results (e.g., the returned record objects) for a given matching strategy 1100 can be appended to the record object array 1202 for the associated record object type. For example, matching strategy 1100(1) can be used to return the account record objects with UIDs A1 and A17, the matching strategy 1100(2) can be used to return the account record object with the UID A93, and the matching strategy 1100(3) can be used to return the account record object with the UIDs A123 and A320.

The recipient-side matching strategies 1100 can include a plurality of matching strategies. The matching strategies can be arranged in a predetermined and configurable order. The matching strategies of the recipient-side strategies 1100 can include one or more of matching to opportunity record objects based on contact role, matching to account record objects based on contact record objects, matching to account record objects based on domains, matching to opportunity record objects based on contacts, matching to partner account record objects based on contacts, matching to partner account record objects using domains, among others. The record object identification engine 315 can use the recipient-side strategies 1100 to select a plurality of candidate record objects to form record object arrays 1202.

Each value in the matched record object arrays 1202 can include an indication of one of the record objects that was matched using the matching strategies (e.g., the recipient-side strategies 1100). For example, the matched record object arrays 1202 can include an array of UIDs associated with each of the record objects that were matched by the record object identification engine 315 using the matching strategies. In some embodiments, each value in the array can be a data pair that includes the matched record object UID and a score indicating how confident the system is on the match between the electronic activity and the record object. The score can be based on the matching strategy which returned the given record object. In some embodiments, the score may be adjusted based on previous matches and how a user accepted or modified the previous matches. In some embodiments, a record object can be selected multiple times; for example, a first and a second matching strategy can each select a given record object. A score can be associated with each matching strategy and the score for the record object selected by multiple matching strategies can be an aggregate (for example, a weighted aggregate) of the scores associated with each of the matching strategies that selected the record object.

The scores can indicate how well the selected record object satisfied the one or more matching strategies.

The record object identification engine 315 can select record objects based on matching strategies for each of the participants associated with the electronic activity. For example, the electronic activity can be an email with a sender and a plurality of recipients. The sender and the plurality of recipients can be the participants that are associated with the electronic activity. The record object identification engine 315 can apply each of the matching strategies for each of the participants. Multiple matching strategies for a given participant can return the same record object multiple times. A matching strategy applied to multiple participants can return the same record object multiple times. The score that the record object identification engine 315 assigns to each selected record object can be based on the number of times the given record object was returned after the matching strategies were applied for each of the electronic activity's participants. For example, a first record object can be returned or selected four times and a second record object can be returned or selected once. The record object identification engine 315 can assign the first record object a higher relative score than the second record object that was only selected once.

In some embodiments, the record object identification engine 315 can select record objects using matching strategies that select record objects based on tags. The electronic activity can be parsed with a natural language processor and the tags can be based on terms identified in the electronic activity. Parsing the electronic activity with the natural language processor can enable the electronic activity to be matched to record objects by mention. For example, the electronic activity can be parsed and the term “renewal” can be identified in the electronic activity. A “renewal” tag can be applied to the electronic activity. A matching strategy to select record objects based on tags can select a renewal record object opportunity with the electronic activity and include the renewal record object opportunity in the record object array 1202. In another example, the system 200 can identify identification numbers contained in the electronic activity for which tags can be assigned to the electronic activity. The identification numbers can include serial numbers, account numbers, product numbers, etc. In this example, and assuming a tag identifying an account number is assigned to the electronic activity, a matching strategy to select record object based on tags can select an account record object that includes a field with the account number identified in the electronic activity's tag.

The record object identification engine 315 can apply one or more of a plurality of sender-side strategies 1104 that can be used to select one or more candidate record objects included in one or more second set of record object arrays 1204. In some embodiments, the record object identification engine 315 can apply one or more of a plurality of sender-side strategies 1104 to restrict or prune the record objects selected using the matching strategies 1100. By applying the set of sender-side strategies 1104, the record object identification engine 315 can generate the second set of record object arrays 1204 that can be used to prune or restrict the first set of record object arrays 1202. For example, the record object identification engine 315, applying a sender-side strategy 1104 that selects accounts record objects based on an account owner, can select the account record object with UID A17 and A123. The record object identification engine 315 can use sender-side strategies such as selecting record objects for matching based on account teams associated with one or more participants of the electronic activity. For example, the record object identification engine 315 can select a record object that identifies the sender of the electronic activity. as a member of the account team associated with the record object.

The record object identification engine 315 can prune the identified candidate record object by determining the intersection of the first set of record object arrays 1202 (produced with matching strategies 1100) and the second set of record object arrays 1204 (produced with matching strategies 1104). For example, the account record object array 1202 generated with the set of matching strategies 1100 is, in the example illustrated in FIG. 12, {A1, A17, A93, A123, A320}. The account object array 1204 generated with the set of sender-side strategies 1104 is {A17, A123}. The record object identification engine 315 can determine that the intersection array 1206 of the account record object array 1202 and account record object array 1204 is {A17, A123}. In this way, the sender-side strategy restricted the record objects A1, A93 and A320 from being selected as a match to the incoming electronic activity. The record object identification engine 315 can combine the intersection arrays 1206 generated by the intersection of the sender-side strategies 1104 and the recipient-side strategies 1100 to generate an output array 1208. The output array 1208 can include indications of record objects and the weights or scores associated with each of the record objects.

The record object identification engine 315 can also use restriction strategies to further prune or restrict out record objects selected using the matching strategies 1100 and 1104. The record object identification engine 315 can use the restriction strategies to select one or more record objects to which the electronic activity should not be matched. For example, although this example is not reflected in FIG. 12, the record object identification engine 315 can use a restriction strategy to select record objects A1 and A17 to generate a restriction record object array including {A1, A17}. If, using the recipient-side matching strategies, the record object identification engine 315 selects record objects A1, A3, A10, and A17 to generate {A1, A3, A10, A17}, the record object identification engine 315 can remove A1 and A17 from the record object array because they were identified in the restriction record object array as record object to which the electronic activity should not be matched.

In some embodiments, the record object identification engine 315 can apply the restriction strategies once the record object identification engine 315 selects one or more record objects with the sender-side strategies 1104 or the recipient-side strategies 1100. The record object identification engine 315 can apply the restriction strategies before the record object identification engine 315 selects one or more record objects with the sender-side and recipient-side strategies. For example, the restriction strategies can be one of the below-described matching filters.

In some embodiments, the output array 1208 can include one or more record objects that can be possible matches for the electronic activity. The selection from the output array 1208 can be performed by the below described record object identification engine 315. If the output array 1208 only includes one record object, the electronic activity can be matched with the record object of the output array 1208. In some embodiments, the electronic activity is only matched with the record object if the confidence score of the record object is above a predetermined threshold. The confidence score of the record object indicates a level of confidence that the record object is the correct record object to which to link the electronic activity. If the output array 1208 includes multiple record objects, the electronic activity can be matched with the record object having the highest confidence score (given that the highest confidence score is above the predetermined threshold). If the output array 1208 does not include any record objects, the confidence score of the record objects are not above the predetermined threshold, or multiple record objects have the same confidence score above the predetermined threshold, the system can request input from the user as to which record object to match the electronic activity. In these cases, the matching strategies can be updated based on the input from the user.

In some embodiments, the record object identification engine 315 can group or link contact record objects on one or both sides of a business process into groups. The record object identification engine 315 can use the groups in the matching strategies. For example, the record object identification engine 315 can group users on a seller side into account teams and opportunity teams. Account teams can indicate a collection of users on the seller side that collaborate to close an initial or additional deals from a given account. Opportunity teams can be a collection of users on the seller side that collaborate to close a given deal. The record object identification engine 315 can add a user to an account or opportunity team by linking the contact record object of the user to the given account team record object or opportunity team record object. The record object identification engine 315 can use account team-based matching strategies or opportunity team-based matching strategies to select record objects with which the electronic activity can be matched.

In some embodiments, at periodic intervals, the record object identification engine 315 can process the electronic activities linked with account record objects and opportunity record objects to generate account teams and opportunity teams, respectively. For a given account record object, the record object identification engine 315 can count the number of times that a seller side user interacts with the account record object (for example, is included in an electronic activity that is linked or matched to the account record object). For example, the record object identification engine 315 can count the number of times the user was included on an email or sent an email that was linked with the account record object. If the count of the interactions is above a predetermined threshold, the record object identification engine 315 can add the user to an account team for the account record object. In some embodiments, the count can be made over a predetermined time frame, such as within the last week, month, or quarter. The record object identification engine 315 can perform a similar process for generating opportunity teams. In some embodiments, the account teams and opportunity teams can be included in the matching and restriction strategies used to match an electronic activity with a record object. Conversely, if the count of the interactions of a particular user is below a predetermined threshold within a predetermined time frame (for example, a week, a month, three months, among others), the record object identification engine 315 can remove the user from the account team or the opportunity team.

In some embodiments, the record object identification engine 315 can select record objects with which to match a first electronic activity based on a second electronic activity. The second electronic activity can be an electronic activity that is already linked to a record object. The second electronic activity can be associated with the first electronic activity. For example, the system 200 can determine that the first and second electronic activities are both emails in a threaded email chain. The system can determine the emails are in the same thread using a thread detection policy. The thread detection policy can include one or more rules for detecting a thread by comparing subject lines and participants of a first email and a second email or in some embodiments, by parsing the contents of the body of the second email to determine if the body of the second email includes content that matches the first email and email header information of the first email is included in the body of the second email. If the second electronic activity is an earlier electronic activity that is already matched to a given record object, the record object identification engine 315 can match the first electronic activity to the same record object.

The policy engine 320 can be any script, file, program, application, set of instructions, or computer-executable code that is configured to enable a computing device on which the policy engine 320 is executed to manage, store, and select matching strategies. The policy engine 320 can generate, manage, and store one or more matching strategy policies for each of the data source providers. For example, the policy engine 320 can generate matching strategy and restriction strategy policies for each division or group of users within a data source provider.

In some embodiments, a matching policy can include a data structure that indicates which matching strategies to apply to an electronic activity for a given data source provider. For example, the matching policy can include a list of matching strategies that are used to select record objects. The list of matching strategies can be manually created by a user or automatically generated or suggested by the system. In some embodiments, the policy engine can learn one or more matching strategies based on observing how one or more users previously matched electronic activities to record objects. These matching strategies can be specific to a particular user, group, account, company, or across multiple companies. In some embodiments, the policy engine can detect a change in linkages between one or more electronic activities and record objects in the system of record (for example, responsive to a user linking an electronic activity to another object inside a system of record manually). The policy engine can, in response to detecting the change, learn from the detected change and update the matching strategy or create a new matching strategy within the matching policy. The policy engine can be configured to then propagate the learning from that detected change across multiple matching strategies corresponding to one or more users, groups, accounts, and companies. The system can also be configured to find all past matching decisions that would have changed had the system detected the user-driven matching change before, and update those matching decisions retroactively using the new learning.

In some embodiments, the matching policy can also identify which restriction strategies to apply to an electronic activity for a given data source provider. For example, the restriction policy can include a list of restriction strategies that are used to restrict record objects. The list of restriction strategies can be manually created by a user or automatically generated or suggested by the system. In some embodiments, the policy engine can learn one or more restriction strategies based on observing how one or more users previously matched or unmatched electronic activities to record objects. These restriction strategies can be specific to a particular user, group, account, company, or across multiple companies. In some embodiments, the policy engine can detect a change in linkages between one or more electronic activities and record objects in the system of record (for example, responsive to a user linking or unlinking an electronic activity to another object inside a system of record manually). The policy engine can, in response to detecting the change, learn from the detected change and update the restriction strategy or create a new restriction strategy within the restriction policy. The policy engine can be configured to then propagate the learning from that detected change across multiple restriction strategies corresponding to one or more users, groups, accounts, and companies. The system can also be configured to find all past matching decisions that would have changed had the system detected the user-driven restriction change before, and update those matching decisions retroactively using the new learning.

The policy engine 320 can update the matching policy with input or feedback from the data source provider to which the matching policy is associated. For example, the data source provider can provide feedback when an electronic activity is incorrectly linked and the matching policy can be updated based on the feedback. Updating a matching policy can include reordering the matching strategies, adding matching or restriction strategies, adjusting individual matching strategy behavior, removing matching strategies, or adding restriction strategies. The link restriction engine 330 can be any script, file, program, application, set of instructions, or computer-executable code, that is configured to enable a computing device on which the link restriction engine 330 is executed to limit to which record objects an electronic activity can be linked.

In some embodiments, data source providers can generate restriction policies or restriction strategies that include rules that indicate conditions under which electronic activities should not be linked to record objects. For example, restriction policies can include rules that prevent internal emails from being linked to a record object. Other restriction policies can limit bot emails (e.g., emails sent to a plurality of people or an email sent as an automatic reply), non-person electronic activity (e.g., electronic activity, such as calendar activity, associated with an asset, such as a conference room), activities, related to persons, who are working in sensitive or unrelated positions (e.g. HR employees), activities, related to persons who do not “own” specific records in the system of record or who do not belong to specific groups of users, or to private or personal electronic activities (e.g., non-work-related emails). These restriction policies or restriction strategies can include one or more matching filters described herein.

The restriction policies can be generated automatically by the system or can be provided by the data source provider. Different restriction policies can be linked together to form a hierarchy of restriction policies, preserving the order in which they should be applied. For example, restriction policies can be set and applied at a group node level (e.g., company level), member node level (e.g., user level), account level, opportunity level, or team level (e.g., groups of users such as account teams or opportunity teams). For example, a restriction policy applied at the company level can apply to the electronic activity sent or received by each employee of the company while a restriction policy applied at the user level is only applied to the electronic activity sent or received by the user.

The link restriction engine 330 can use the restriction policies to remove or discard record objects from the output array 1208. For example, if a restriction policy indicates that electronic activity from a given employee should not be linked to record object A17 and record object A17 is included in the output array 1208, the link restriction engine 330 can remove record object A17 from the output array 1208.

In some embodiments, the link restriction engine 330 can apply the restriction policies to electronic activities prior to the matching performed by the record object identification module 315. For example, if a restriction policy includes rules that calendar-based electronic activity for a conference room should not be linked to any record object, the link restriction engine 330 can discard or otherwise prevent the record object identification module 315 from linking the electronic activity to a record object.

The tagging engine 265 can be any script, file, program, application, set of instructions, or computer-executable code that is configured to enable a computing device on which the tagging engine 265 is executed to generate tags for the electronic activity. The tagging engine 265 can generate or add tags to electronic activity based on information generated or otherwise made available by the record object identification module 315 and the matching model 340. The tagging engine 265 can generate a tag array that includes each of the plurality of tags assigned or associated with a given electronic activity. By having tags assigned to electronic activities the node graph generation system 200 can be configured to better utilize the electronic activities to more accurately identify nodes and record objects to which the electronic activity should be linked.

In addition to the above described tags, the tagging engine 265 can assign tags to an electronic activity based on the output of the record object identification module 315 and matching model 340, among other components of the system described herein. For example, the tagging engine 265 can add one or more tags indicating to which record objects the record object identification module 315 returned as candidate record objects for the electronic activity. For example, and also referring to FIG. 12, the tagging engine 265 can add tags to indicate each record object contained within the output array 1208. In some embodiments, the tagging engine 265 can add a tag for each record object contained within the output array 1208. In some embodiments, the tagging engine 265 can add a tag for each record object contained within the output array 1208. In some embodiments, the tagging engine 265 can include a tag only for the record object in the output array 1208 that most closely matches the electronic activity.

The linking generator 335 can be any script, file, program, application, set of instructions, or computer-executable code that is configured to enable a computing device on which the linking generator 335 is executed to link electronic activities to record objects. As described above, the system can generate and maintain a shadow system of record for each of a data source provider's system of record. The data source provider's system of record can be referred to as a master system of record or tenant-specific system of record. The linking generator 335 can select a record object from the record object array 1208 and link the electronic activity to the selected record object in the shadow system of record. For example, the record object identification engine 315 can use the confidence scores of the record objects in the record object array to select a record object with which to match the electronic activity.

Also referring to FIG. 12, the linking generator 335 can link the electronic activity to one or more of the record objects included in the output array 1208. In some embodiments, the linking generator 335 can link the electronic activity to one or more record objects in the output array 1208. For example, the linking generator 335 may only link the electronic activity to the record object in the output array 1208 that most closely matches the electronic activity. In some embodiments, the linking generator 335 links the electronic activity with only one of the record objects in the output array 1208 (e.g., the record object having the highest score).

Linking the electronic activity with a record object can include saving the electronic activity (or an identifier thereof) into the shadow system of record in association with the record object. For example, each record object can include a unique identifier. The electronic activity can be saved into the system of record and the record object's unique identifier can be added to a record object field of the electronic activity to indicate to which record object the electronic activity is linked. In some embodiments, each electronic activity can be assigned a unique identifier. The electronic activity's unique identifier can be added to a field in the shadow record object to indicate that the electronic activity is associated with the shadow record object. In some embodiments, the shadow record object can be matched or synced with a record object in a client's system. When the shadow record object and the record object are synced, data, such as the electronic activity's unique identifier in the above example, can be copied to the corresponding field in the matched record object of the client's system. For example, if the linking generator 335 matches an email to a given record object in the shadow system of record, when synced the email can be matched to the given record object in the client's system of record.

By linking the electronic activities to record objects, the system can generate metrics regarding the electronic activities. The metrics can include engagement scores for users, employees, specific deals or opportunities, managers, companies, or other parties associated with a system of record. Additional details regarding metrics and the calculation thereof are described below in Section 11, among others. The engagement scores can indicate amongst other things how likely an opportunity (or deal) is to close successfully (or unsuccessfully) or whether the number of contacts in the account are sufficiently engaged with the sales representative to prevent the account from disengaging with the company. The engagement scores can provide an indication of an employee's productivity and can indicate whether the user should receive additional training or can indicate whether the user is on track to achieve predefined goals. The metrics can be calculated dynamically as the electronic activities are matched to nodes and record objects or the metrics can be calculated in batches, at predetermined intervals. Metrics can also be based on the content or other components of the electronic activity in addition to or in place of the linking of the electronic activity to a node and record object.

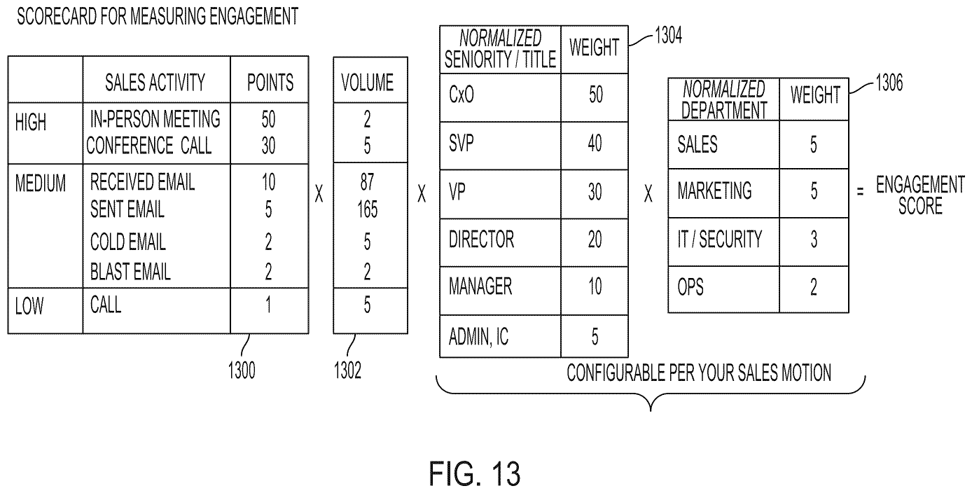

For example, FIG. 13 illustrates an example calculation for calculating the engagement score of an opportunity record object. The example calculation can include an electronic activity weight 1300, a volume vector 1302 indicating a count of each electronic activity type, a seniority weight 1304, and a department weight 1306. As illustrated in FIG. 13, the electronic activity linking engine 250 can determine the engagement score by collecting each of the electronic activities associated with a given opportunity record object. The electronic activity linking engine 250 can count the volume (e.g., number) of each type of electronic activity linked with the opportunity record object. For example, the electronic activity linking engine 250 can tag each ingested electronic activity as being an in-person meeting electronic activity, a conference call electronic activity, a received email electronic activity, a sent email electronic activity, a cold email electronic activity, a blast email electronic activity, or a call, among others. The electronic activity linking engine 250 can also tag the electronic activity using NLP.

For example, electronic activity linking engine 250 can tag an email based on mentions of a competitor, product, specific people, specific places, or other phrases contained within the electronic activity. The electronic activity linking engine 250 can also generate tags based on the combination of other tags, linking information, and fields within linked objects.

The count of each of the different types of electronic activities can be stored in the volume vector 1302. The volume vector 1302 can be multiplied by the weight or points assigned to each of the different electronic activities. The weight or points associated with each of the electronic activity types in the electronic activity weight can indicate the significance of the electronic activity to the successful completion of the deal. In some embodiments, the weights can be set by the electronic activity linking engine 250. The weights can be set based on the sales motion of the given tenant or data source provider. Each weight can be a normalized value that can represent the significance a given feature, or collection of electronic activities. For example, an email including the VP of Sales can be given a higher weight when compared to an email that only includes managers. In some embodiments, the electronic activity linking engine 250 can reference an organizational hierarchy derived from the node graph and assign relatively higher weights to electronic activities that involve people relatively higher in the organizational hierarchy. For example, having repeated, in-person meetings with a CxO at a prospective client or company can be more beneficial to the successful closing of the deal than cold calling a random contact at the company. Accordingly, the in-person meeting is assigned a higher weight (50 points) that the call, which is assigned a relatively lower weight of 1.

The engagement score can also be based on a seniority weighting factor. The seniority weighting factor can then be applied to the volume weighted scores of the electronic activities. The seniority weighting factor can apply a weighting based on those included on or involved with the electronic activity. In some embodiments, the feature extraction engine 310 can determine which contacts or people are associated with electronic activity. For example, the feature extraction engine 310 can parse the TO: and CC: fields of an email (an example electronic activity) and then, using the node graph, determine seniority, department, job title, or role of each contact listed on the email at their current and past roles. In some embodiments, the seniority weighting factor can be based on the contact record objects to which the matching model 340 (or other component of the system) matched the electronic activity.

The engagement score can also be based on a department weighting factor. The department weighting factor can be normalized across all the departments (such as within a company or account). In some embodiments, once the system determines which contacts are associated with the electronic activity, as described above, the system can determine the department of each of the contacts using the node graph.

The stage classification engine 325 can be any script, file, program, application, set of instructions, or computer-executable code, that is configured to enable a computing device on which the stage classification engine 325 is executed to determine or predict a stage of a deal or opportunity.

In some embodiments, record objects can be associated with a plurality of stages. In some embodiments, the record object can be an opportunity record object or any other record object that describes a business process, such as a sales process, a hiring process, or a support ticket. The stages can be defined by the system or by the data source provider.

Using the example of an opportunity record object in a sales process, the stages can indicate the steps taken in an opportunity or deal from the beginning of the deal to the final disposition of the deal (e.g., close and won or closed and lost). The stages can include, but are not limited to: prospecting, developing, negotiation, review, closed/won, or closed/lost.

Each of the stages can be linked to different tasks or milestones. For example, a sales representative can develop a proposal during the “developing” stage. Each of the stages can be linked to different actions taken by the sales representative or prospect contacts, associated contacts or other people. For example, initially during the prospecting and developing stages a sales representative may be involved in the opportunity or deal. At a later stage, such as negotiations, a sales manager may become involved in the deal.

The stages can be based on the contacts present or involved on both sides of the deal. For example, as the deal advances to higher stages, more senior people may be included in the electronic activities. The stage of the deal can be based on the identification or introduction of an opportunity contact role (OCR) champion. In some embodiments, an administrator or user of the system of record can link the opportunity record object with a contact record object and designate the contact of the contact record object as an opportunity contact role. The champion can be a person on the buyer side of the deal that will support and provide guidance about the deal or opportunity to the seller side. In some embodiments, the OCR champion can be selected based on one or more rules. For example, the one or more rules can include setting the person identified as the VP of sales (or other specific role) as the OCR champion. In some embodiments, the OCR champion can be selected based on historical data.

For example, the historical data can indicate that in 90% of the past deals a specific person or role was the OCR champion. Based on the historical data, when the person is added as a recipient of an electronic activity, the person can be identified as the OCR champion. The OCR champion can also be identified probabilistically based on tags associated with the electronic activities linked to the opportunity record object or content within the electronic activities.

In some embodiments, OCRs can be configurable by the company on an account by account basis. Depending on the type, size or nature of the opportunity, the customer or account involved in the opportunity may have different types and numbers of OCRs involved in the opportunity relative to other opportunities the same customer is involved in. Examples of OCRs can include “Champion,” “Legal,” “Decision Maker,” “Executive sponsor” among others.

The system 200 can be configured to assign respective opportunity contact roles to one or more contacts involved in an opportunity. The system 200 can be configured to determine the opportunity contact role of a contact involved in the opportunity based on the contact's involvement. In some embodiments, system 200 can determine the contact's role based on a function the contact is serving. The function can be determined based on the contact's title, the context of electronic activities the contact is involved in, and other signals that can be derived from the electronic activities and node graph. In addition, the system 200 can assign the contact a specific opportunity contact role based on analyzing past deals or opportunities in which the contact has been involved and determining which opportunity contact role the contact has been assigned in the past. Based on historical role assignments, the system 200 can predict which role the contact should be assigned for the present opportunity. In this way, the system 200 can make recommendations to the owner of the opportunity record object to add contacts to the opportunity or assign the contact an opportunity contact role.

In some embodiments, the system 200 can determine that a contact should be assigned an opportunity contact role of “Executive Sponsor.” The system may determine this by parsing electronic activities sent to and from the contact and identify, using NLP, words or a context that corresponds to the role of an Executive sponsor. In addition, the system can determine if the contact has previously been assigned an opportunity contact role of executive sponsor in previous deals or opportunities. The system can further determine the contact's title to determine if his title is senior enough to serve as the Executive sponsor.

In some embodiments, the electronic activity linking engine 250 can use a sequential occurrence of electronic activities to determine contact record objects that should be linked or associated with an opportunity record object. The electronic activity linking engine 250 can also determine the roles of people associated with the contact record objects linked to an opportunity. The identification of people associated with opportunity and account record objects (and their associated roles) can be used to determine stage classification, group of contacts on the buyer side that are responsible for the purchase, and for many other use cases. In some embodiments, the sequential occurrence of electronic activities can be used to determine the role or seniority of users involved in a business process. For example, initial emails linked with an opportunity record object can involve relatively lower-level employees. Later emails linked to the opportunity record object can include relatively higher-level employees, such as managers or Vice Presidents. The electronic activity linking engine 250 can also identify the introduction of contacts in a chain of electronic activities, such as a series of email replies or meeting invites, to determine a contact's participation and role in a business process. For example, the electronic activity linking engine 250 can use NLP and other methods to identify the introduction of a manager as a new OCR based on an email chain.

It should be appreciated that in some embodiments, the node graph generation system 200 can include node profiles corresponding to each of the contact record objects included in one or more shadow system of records or master systems of records. As sequential electronic activities traverse the system 200, the node graph generation system 200 can parse the electronic activities and determine that additional email addresses are being included or some existing email addresses are being removed in subsequent electronic activities. The node graph generation system can identify node profiles corresponding to the email addresses being added and establish links or relationships between the node profiles included in the electronic activity. As the electronic activity linking engine 250 links electronic activities to record objects, such as opportunity record objects, node profiles included in the electronic activity are also linked to the opportunity record object. The stage classification engine can use this information to classify a stage of the opportunity based in part on node profiles linked to the record object and based on the involvement of the node profiles in the electronic activities that can be determined using effort estimation techniques, volumes of emails exchanged, as well as based on NLP of the content to identify the role of each of the node profiles, as well as historical patterns of linkage of similar node profiles to similar record objects, as discussed below.

In some embodiments, the electronic activity linking engine 250 can also determine a contact's role based on the tags of the electronic activity in which the contact was included. For example, relatively higher-level employees, such as managers, can be more likely to be included electronic activities such as in person meeting invites and conference calls. The electronic activity linking engine 250 can also use NLP on the content of electronic activities to determine the role of contacts. For example, the electronic activity linking engine 250 can process the content of the electronic activities to identify terms that may indicate a role of a contact. For example, an email can include the phrase “my assistant Jeff will schedule the meeting.” The electronic activity linking engine 250 can identify the phrase “my assistant Jeff” and include in the contact record object associated with Jeff the role of “assistant.” The electronic activity linking engine 250 can also determine that the sender of the email is more likely to be a manager because the sender of the email has an assistant.

Similar to how the record object manager 255 maintains the shadow systems of record and corresponding record objects, the stage classification engine 325 can maintain a shadow stage indicating a stage the stage classification engine 325 determines is the current stage for the deal or opportunity. The stage classification engine 325 can determine or estimate the stage of the opportunity using a top-down algorithm or a bottom-up algorithm. With the top-down algorithm, the data source provider can provide a policy that includes a plurality of rules. The rules can indicate requirements for entering or exiting a stage. For example, the data source provider's policy may include a rule indicating that an opportunity cannot progress to a negotiation stage until a procurement manager is involved in the deal on the buyer's side. In this example, the stage classification engine 325 can monitor the ingested electronic activities. When the stage classification engine 325 detects that the system has linked an electronic activity (such as an email) to the opportunity record object and the electronic activity includes a contact that is a procurement manager (as determined, for example, via the node graph), the stage classification engine 325 can set the shadow stage to negotiation stage. In some embodiments, the shadow stage can be synced to the data source provider's stage for the given record object. In some embodiments, the stage classification engine 325 can update a stage of a record object of the master system of record to match the shadow stage of the corresponding record object determined by the stage classification engine 325. In some such embodiments, the client may provide or select a configuration setting that allows the stage classification engine 325 to update the stage classification of a record object of the master system of record of the client. In some embodiments, the stage classification engine 325 can use a bottom-up approach to predict or determine the stage. The stage classification engine 325 can use machine learning to predict or determine the stage of a deal or opportunity. For example, the stage classification engine 325 can combine the features from each of the electronic activities linked to an opportunity record object into a feature vector. The stage classification engine 325 can use a neural network, or other machine learning technique, to classify the deal into one of the stages based on the feature vector. The machine learning algorithm can be trained using the progression of previous deals through the stages. In some embodiments, the stage classification engine 325 can map the feature vector and plurality of electronic activities to a specific stage as defined by the data source provider. In some embodiments, the stage classification engine 325 can map the feature vector and plurality of electronic activities to a normalized stage as defined by the system. The normalized stages can be used with different data source providers to provide a translatable staging system or nomenclature across the different data source providers. The stage classification engine 325 can maintain mappings between the normalized stages and the stages of the different data source providers. For example, the stage classification engine 325 can define five, normalized stages. A first data source provider can define a deal or opportunity as including 7 stages. A second data source provider can define a deal or opportunity as including 3 stages. The stage classification engine 325, for the first data source provider, may map stages 1 and 2 to normalized stage 1, stage 3 to normalized stage 2, stage 4 to normalized stage 3, stage 5 to normalized stage 4, and stages 6 and 7 to normalized stage 5. Accordingly, the data source provider's stages can be mapped to the normalized stages based on the tasks, requirements, or content of the stages rather than by the naming or numbering of the stages.

The stage classification engine 325 can map the electronic activities or feature vector to one of the five normalized stages. The indication of which normalized stage the electronic activities or feature vector was mapped to can be saved as a shadow stage. When syncing the shadow stage to the master stage of the data source provider, the stage classification engine 325 can map each of the normalized stages to the stages as defined by the data source provider. For example, the first normalized stage may be mapped to the first stage as defined by the data source provider and the second normalized stage may be mapped to the second and third stages as defined by the data source providers.

### 3. Systems and Methods for Linking Electronic Activities to Record Objects Maintained on Systems of Record

As described above, the system can maintain one or more shadow systems of record and shadow stages for each of the data source providers. The shadow systems of record can mirror the data source provider's systems of record at different instances in time. In some embodiments, as described above, electronic activities ingested by the system from a given data source provider are linked to the data source provider's shadow systems of record to enable the system to perform analysis and generate metrics regarding the data source provider's systems of record. In some embodiments, the system can synchronize the linked electronic activities between the shadow systems of record and the data source provider's master systems of record.

The record object manager 255 can maintain data regarding the record objects in the shadow systems of record and the master systems of record. The record object manager 255 can synchronize shadow systems of record and master systems of record for each of the data source providers. In some embodiments, to synchronize the shadow systems of record and the master systems of record the record object manager 255 can detect changes in the master systems of record. The changes can include added, deleted, or modified account record objects, opportunity record objects, or lead record objects or any other record objects. For example, the record object manager 255 can determine that a new account record object was generated at the master system of record and generate a corresponding copy of the new account record object at the shadow system of record. The corresponding copy of the new account record object at the shadow system of record can be a copy of the new account record object at the master system of record. Responsive to adding the new record object, the system can reprocess previously processed electronic activities to determine if the electronic activities should be matched with the new record object.

Detecting if modifications occurred to the record objects of the master system of record can include determining if one or more fields of the record object changed or if the linking of electronic activities with the record object changed. For example, during a previous synchronization cycle the record object manager 255 could link an electronic activity with a first record object at the master system of record. After the synchronization, a user at the master system of record may modify linkage to link the electronic activity with a second record object. In another example, the system can detect that an additional field value was added. For example, location data can be added to location field of a record object. The record object manager 225 can resynchronize the updated record object to identify potential new matches based on the added location data. The system can also reevaluate previous matches and determine if the location data makes the match with the previous matches more or less likely. The record object manager 255 can determine that the electronic activity was linked by the user to a different record object. The record object manager 255 can provide an indication of the change to the record object identification module 315 as feedback so that matching model 340 can update its machine learning models or matching strategies. In some embodiments, a user can add additional information or change information in a record object. Responsive to the change to the record object, the system can perform the rematching of the electronic activity with nodes and record objects.

The record object manager 255 can synchronize changes to the shadow systems of record to the master systems of record. For example, new linkings of electronic activities to record objects can be synchronized to the master system of record. Synchronizing the shadow system of record to the master system of record can include adding any linked electronic activities since the last synchronization cycle to the master system of record. The electronic activities can be linked to the same record object in the master system of record to which they are linked in the shadow system of record. In some embodiments, the record object manager 255 can add a flag or tag to the electronic activity when the electronic activity is synchronized from the shadow system of record to the master system of record. The flag can include an indication that the electronic activity was synchronized from the shadow system of record. In some embodiments, setting of the flag can cause the master system of record to prompt a user of the master system of record to confirm that the electronic activity was linked to the correct record object. In some embodiments, setting of the flag can cause the master system of record to provide a visual indication to a user of the master system of record that the flagged electronic activity was linked and synchronized from a shadow system of record. In some embodiments, the user can confirm or decline the addition of the linked electronic activity from the shadow system of record. Based on the approval or disapproval of the linked electronic activity, the system can update the matching strategies.

### 4. Systems and Methods for Generating a Multi-Tenant Master Instance of Systems of Record Using Single-Tenant Instances

In some embodiments, the system 200 or the system 9300 shown in FIG. 3 as described herein can generate a multi-tenant master instance of the systems of record. The multi-tenant master instance of the systems of record can include data from a plurality of master systems of record from a plurality of different data source providers, which can be referred to as tenants, or from the plurality of shadow systems of record, which can themselves be mirrors or copies of master systems of record from the different tenants. In some embodiments, the multi-tenant master instance of the systems of record can be a combination of the record objects from the separate shadow systems of record.

As described herein, the system 200 or the system 9300 shown in FIG. 3 can include shadow systems of record that correspond to respective master systems of record belonging to respective data source providers. In some embodiments, each of the shadow systems of record (and corresponding master systems of record) can include a plurality of record objects. The record object manager 255 can synchronize the record objects (or data therein) from each of the shadow systems of record or master systems of record from different tenants into a multi-tenant master instance of the systems of record. As such, the multi-tenant master instance of the systems of record can include all of the data included in each record object of the one or more shadow systems of record and the corresponding master systems of record. The multi-tenant master instance of the systems of record can be used to further enrich the node profiles maintained by the node profile manager 220.

The multi-tenant master instance of the systems of record maintained by the system 200 or the system 9300 shown in FIG. 3 can be used to synchronize data between the master systems of record from the different tenants as well as improve the multi-tenant master system of record and individual master systems of record of the data source providers using parsed and normalized activity data received from electronic communications servers of the data source providers. Moreover, the system can update one or more node profiles maintained by the node profile manager 220 using the data from the record objects of the one or more master systems of record. The record object manager 255 can sync fields or data between node profiles and record objects such as, but not limited to, names, phone numbers, email address, domains, other contact information, address, D-U-Ns numbers, job titles, department IDs and other standard company or person information. In some embodiments, some types of systems of record can include record object (and data) types that are not included in other types of systems of record such that one or more of the systems of record may not support all record object types or data types maintained in the multi-tenant master system or record.

The record object manager 255 can populate data from the record objects from the individual master systems of record into the multi-tenant master instance of the systems of record. The record object manager 255 can also be configured to synchronize the record objects (or data contained therein) from the multi-tenant master instance of the systems of record back to the individual shadow systems of record enabling data to be shared between the different tenants. In some embodiments, each shadow system of record can include data that is obtained from a corresponding master system of record of a specific data source provider. This data can be shared with or accessed by the record object manager 255, which can use the data from each of the shadow systems of record to update the multi-tenant master instance of the systems of record. Moreover, the record object manager 255 can further update the record objects included in the multi-tenant master instance of the systems of record from the node profiles of the nodes maintained by the node profile manager 220. The record object manager 255 can then use the data included in the multi-tenant master instance of the systems of record, which has been updated from multiple systems of records and the node profiles, to update one or more of the shadow systems of records, which can then be used to update the corresponding master systems of records of the data source providers.

Data source providers or tenants that provide access to their systems of record can establish, via the system 9300, one or more controls or settings to manage how the data in their respective systems of record are treated. In some embodiments, a tenant can select a setting that restricts the system 9300 from using the information included in the tenant's system of record to update the master instance of the systems of record maintained by the system 9300. In some embodiments, a tenant can select a setting that restricts the system 9300 from using the information included in the tenant's system of record to update systems of record of other tenants maintained by the system 9300. Furthermore, in some embodiments, a tenant can select a setting that restricts the system 9300 from using only certain information, such as sensitive or competitive information included in the tenant's system of record to update the master instance of the systems of record maintained by the system 9300. The system 9300 can provide individual tenants control as to how the data included in a tenant's system of record can be updated, used and shared. For instance, a tenant can select a configuration setting that restricts the system 9300 from updating the tenant's system of record.

Each record object can include a plurality of fields that are populated with data regarding a given record object. As one example, a contact record object can include fields for first name, last name, email, mobile phone number, office phone number, among others. A user can populate the fields of the contact record object at the master system of record of one of the tenants (e.g., one of the data source providers). The record object manager 255 can synchronize the populated fields into the corresponding fields of the record object in the shadow system of record. The node profile manager 220, described herein, can generate a first node (e.g., a member node). The node profile manager 220 can populate the fields of the first node with the data from the contact record object. In this example, a second user can populate the fields of a second contact record object in a second master system of record of a different tenant. Once synchronized to the system, the node profile manager 220 can generate a second node based on the second record object. In some embodiments, the node resolution engine 245 can determine that the first node and the second node are associated with the same contact. For example, the node resolution engine 245 can determine that the email fields of the first and second nodes are populated with the same email address. Determining that the first and second nodes are associated with the same contact, the node resolution engine 245 can merge the first and second nodes such that the merged node includes data from both the first and the second nodes. The record object manager 255 can sync the merged fields back to the respective record objects and master systems of record.

For example, and continuing the above example, the first user may have entered a phone number into a contact field but not a department identifier into a department field of the first user's respective contact record object. The second user may have entered the department identifier into the department field but not the phone number into the second user's respective contact record object. The record object manager 255 can determine the two contact record objects are associated with the same person and merge the data into the multi-tenant master instance of the systems of record maintained by the system 200. In some embodiments, the node profile manager 220 can generate a node for the person in the node graph. To sync or otherwise update the merged data back to the respective contact record objects in the corresponding shadow system of record or the corresponding master system of record, the record object manager 255 can update the first user's contact record object with the department identifier and the second user's contact record object with the phone number. In some embodiments, the record object manager 255 can set a flag indicating the multi-tenant master instance of the systems of record as the source of the updated data in the record objects.

When syncing data between the different tenant systems of record and the multi-tenant master instance of the systems of record, the record object manager 255 can resolve conflicts between record objects and field values in the different systems of record that include different data. The record object manager 255 can resolve the conflicts using the above-described node graph. For example, the record object manager 255 can select between conflicting data by selecting the data that has highest likelihood of being accurate. The system 200 can, via the node profile manager 220, maintain confidence scores of different values of fields to determine a likelihood of the value being accurate. In some embodiments, two values of the same field may both be accurate except one may be more current than the other. In such embodiments, the record object manager 255 can select the value that is accurate and more current. As described herein, a confidence score of a value can be based on contribution scores of one or more data points serving as evidence for the value. The contribution scores of the data points can be based in part on a recency of the data point and a trust score of the source indicating how trustworthy the source is. The trustworthiness of a source, such as a system of record, can be based on a health score of the source, which can be determined based on how many values of record objects of the system of record match values the system 200 knows to be true or accurate and how many values of the record objects do not match values the system 200 knows to be true or accurate.

The record object manager 255 can also resolve conflicts based on the time series of the data for the respective fields. For example, an email field that was recently updated by a user may indicate that the contact recently changed their email address and that the newer email address is an updated email address and not an inaccurate email address. Furthermore, such data may be re-confirmed by extracting the newer email address from an email signature in an electronic activity received from an electronic communications server associated with one of the data source providers. In some embodiments, the record object manager 255 can periodically execute batch jobs to synchronize the shadow and master systems of record. For example, each evening the record object manager 255 can synchronize the shadow and master systems of record. When synchronizing the record objects, the record object manager 255 can reprocess previously synced record objects (and the fields therein) to determine if the record objects should be updated. For example, based on the electronic activities processed during the day, the confidence score associated with a value of a field of a record object in the shadow system of record may have decreased below a predetermined threshold and the record object manager 255 can remove the value from the field of the record object of the shadow system of record during the daily sync.

In some embodiments, the synchronization between from the shadow system or record to the master system of record can be governed by privacy policies. For example, electronic activities, record objects, or data contained therein can be flagged to be labeled as private by the system or a user and may not be synced to the master system of record or to other tenant systems of record. In some embodiments, for little known or possibly sensitive data, the system may not sync fields back to systems of record until the data in the field is identified in a predetermined number of systems of record. For example, if a contact record object for John Smith from a first tenant lists the cell phone of John Smith, the cell phone number may not be synced to other tenants' master systems of record until the system 200 identifies the cell phone number in the contact record object of a predetermined number (e.g., 3) of tenant master systems of record, meaning that at least 2 other companies, connected to the system 200 also possess the phone number for John Smith.

### 5. Systems and Methods for Monitoring Health of Systems of Record

In some embodiments, the system described herein can be used to monitor the health of a system of record. The source health scorer 215 can monitor the health of the system of record and can calculate a health score for the system of record. The health score for the system of record can be used to determine or otherwise calculate a trust score for the system of record.

The health (or health score) of a system of record can provide an indication of the accuracy or completeness of a system of record's data. In some embodiments, the health score can be calculated with respect to the given system of record. For example, the health score can indicate that 20% of the records within the system of record are inaccurate. In some embodiments, the health score can be calculated with respect to the other data processing systems. For example, the health score can indicate that the completeness of the systems of record' database is in the 97th percentile when compared to the completeness of other systems of record.

The health score can be based on the completeness of data in the system of record and/or the accuracy of the data in the system of record. For example, each record object in a system of record can include a plurality of fields. In some embodiments, the completeness of the system of record can be based on the ratio of the total number of populated standard fields to the total number of unpopulated standard fields. In some other embodiments, the completeness of the system of record can be based on the ratio of the total number of populated standard and supplemental fields to the total number of unpopulated standard and supplemental fields. In some embodiments, fields of record objects in systems of record can be classified as standard fields if they are common among different systems or record. Examples of standard fields can include company name, company phone number, company address as record objects across different systems of records for the same company may each include this information. Similarly, for record objects directed towards individuals, the standard fields can include first name, last name, work phone number, title as record objects across different systems of records for the same individual may each include this information. Other fields that are not standard fields can include custom fields or fields that include supplemental information that is not common across different systems of record can be classified as supplemental fields. Examples of supplemental fields can include fields such as opportunity contact role, years of experience, industry, as these fields may not be common across multiple systems of record.

In some embodiments, the health score can be based on the total count of the fields that are populated or just the total count of the standard fields that are populated. In some embodiments, the health score can also be based on the accuracy of the data populated into the standard fields. The system can determine the accuracy of the data in the standard fields by comparing the data to other instances of the data in other systems of record or in the multi-tenant master instance. For example, the system can determine that the first tenant system of record indicates a phone number for a given contact is 555-5555. A second and third tenant system of record can indicate that the phone number for the given contact is 555-4433. The system can determine that the phone number in the first tenant system of record is incorrect or not current because more tenants (with health scores satisfying a certain threshold) include the 555-4433 phone number. The accuracy of the data can also be based on the health score associated with data source from which the data was received. For example, the phone number may not be changed when contradicted by a source with a low health score. The accuracy of data can also be based on electronic activities and the confidence score of values of fields maintained in node profiles of the system 200. The accuracy of data included in a system of record can be determined by comparing data included in the record objects of the system of record to information included in corresponding node profiles maintained by the system 200. As described above, the node profiles can be updated with information extracted from electronic activities, which are unbiased and not self-reported or manually entered. Based on the comparison of the data included in the record objects of the system of record and the corresponding node profiles, the source health scorer can determine a health of the system of record. The health score can also be time dependent. For example, the health score can decay with time because the data in the system of record can become stale if the data is not updated or not checked. In some embodiments, newer data can have a greater probability of being accurate. For example, a newly entered job title for a contact may be accurate and indicate a promotion.

In some embodiments, the health score can be based on the links between record objects. For example, the system of record may require that each opportunity record object be linked with a least one contact record object. In these examples, the data fields within the record objects may be complete but the source health scorer 215 can reduce the system of record's health score or assign a lower health score to a system of record responsive to determining that the system of record does not include proper links between one or more opportunity record objects and corresponding contact record objects or any other record objects with which the one or more opportunity record objects should be linked. In some embodiments, the source health scorer 215 can base the health score on the accuracy of the links between the record objects of the system of record. For example, the system can process the electronic activities already linked to the system of record to perform historical matching based on using the techniques described herein to generate predictions for linking between the system or record's record objects. If the linkages between the record objects do not match the predicted matches, the source health scorer 215 can assign the system of record a lower health score.

The source health scorer 215 can also calculate or otherwise determine a trust score for each data point included in an array of a value of a node profile maintained by the system 200 or that contributes towards a value in a record object maintained by the system 200. The trust score can be based on the source of the data point. In some embodiments, the trust score can be based on a health score of the source of the data point. For instance, some systems of record can be better maintained than others. The source health scorer 215 can perform a health check on a system of record to compute a health score for the system of record. The health score of the system of record can be used to assign a trust score. In contrast to data points whose source is a system of record, a data point whose source is an electronic activity ingested by the system 200 can have a higher trust score since electronic activities do not have health related issues as they are not manually input or updated. Systems of record are generally manually input and updated and therefore can include inaccuracies or may be stale resulting in lower health scores, and thereby, lower trust scores. In some embodiments, the source health scorer 215 can assign a trust score of 100% or a maximum rating to data points derived from electronic activities.

6. Systems and Methods for Generating Recommendations to Improve Health Based on a Node Graph Generated from Electronic Activity

In some embodiments, the system described herein can make recommendations based on the health and trust scores associated with a system of record or data source provider. The source health scorer 215 or other components of the system can generate the recommendations based on metrics of the systems of record, record objects therein, and the trust and health scores associated with the systems of record.

The source health scorer 215 can determine, for each field type, of number of standard fields not populated with data. For example, the source health scorer 215 can determine, for a given system of record, that 75% of the contact record objects include domain fields that are not populated with a website field value. In this case, the recommendation can be that the data source provider should update the domain fields of the contact record objects. In some occurrences, the system can automatically fill in a predetermined percentage of the missing field values in a given system of record to automatically improve the health score of the given system of record. Given a significant number of systems of record, connected to the multi-tenant system of record instance and the source health scorer 215, such a system can systematically and continuously improve the health scores of all connected systems of record. Stated in another way, by generating or maintaining a multi-tenant system of record that can be used to update one or more master system of records maintained by customers or enterprises, a network of systems of record are created with automated data entry, thereby allowing each of the master systems of records to get updated. This will result in an improvement in the health and corresponding health score of each of the master systems of record through the network effect until all of the master systems of record are identical and, in some embodiments, pristine or perfect.

In some embodiments, the recommendations can indicate to a data source provider that the data within the system of record is stale or out of date. For example, if a first company is sold to a second company, the system can alert the data source provider to update the company or other information in its systems of record based on the sale of the first company. The recommendations can also include updates to field values, organizational charts, job titles, employment changes, and changes to an organization, such as mergers and acquisitions.

### 7. Systems and Methods for Filtering and Database Pruning

At least one aspect of the present disclosure is directed to systems and methods for filtering and database pruning. For example, the tagging engine 265 can assign tags based on the contents of the electronic activity, associations of the electronic activity with specific nodes, people, or companies, confidence and trust scores, information in record objects, or other information associated with the electronic activity. The rules used by the tagging engine 265 to generate tags can be used by one or more systems or components described herein. In some cases, the rules used by the tagging engine 265 to generate tags can generate filter tags, which can be configured to cause the system to block, delete, remove, drop or redact the electronic activity associated with the filter tag.

A system, such as the data processing system 9300 depicted in FIG. 3, the node graph generation system 200 depicted in FIG. 4, the tagging engine 265 depicted in FIG. 4, the electronic activity linking engine 250 depicted in FIG. 9, or one or more components thereof, may perform significant computationally extensive processing on various types of electronic activities or records as depicted in FIG. 3. Since a large volume of electronic activities associated with sending or receiving electronic activities are received by the systems or components depicted in FIG. 3, 4, or 9 in accordance with the process flow 9302 depicted in FIG. 9, it can be challenging to efficiently process such data without causing excessive delay or latency issues. Further, databases associated with the systems and components depicted in FIGS. 3, 4 and 9, as well as third-party databases with which the systems depicted in FIGS. 3, 4 and 9 can interface or communicate, may store or maintain records that may be stale, sensitive, corrupt, erroneous, or otherwise not needed or not wanted. As such, systems and methods of the present technical solution can provide filtering at an ingestion step 9305 as depicted in the functional flow diagram of FIG. 9302, as well as scrubbing of records maintained in one or more databases, using parsing techniques, rules or machine learning.

The node graph generation system 200 can, via ingestor 205, receive electronic activities. The electronic activities can include, for example, electronic messages or electronic calendar events and associated metadata. The ingestor 205 can receive the electronic activities from one or more data source providers 9350, which can include an electronic messaging or mail server. The ingestor 205, upon receiving the electronic activities, can format the metadata or otherwise manage or manipulate the data to facilitate further processing. The ingestor 205 can receive the electronic activities in real-time, asynchronously, on a periodic basis, based on a time interval, in a batch process or batch download, or responsive to a trigger of event.

The tagging engine 265 can, using one or more rules, policies, or techniques, tag the electronic activities such that the filtering engine 270 can apply a content filter to the tagged electronic activities to determine whether to filter out the electronic activity or authorize or approve the electronic activity for further processing, or redact a portion of the electronic activity. The filtering engine 270 can filter out the electronic activity, which can refer to or include redacting out sensitive or private parts of the electronic communications or preventing the entire electronic activity (or metadata thereof) from being forwarded to another component or memory of the system so that the electronic activity is prevented or blocked from further processing or storage. Preventing the electronic activity from being further processed or stored can reduce unnecessary computing resource utilization or memory utilization as well as prevent sensitive or private information from being carried from systems of record or activity data sources to other systems of record.

The electronic activity parser 210 can provide an alert, tag, notification, label or other indication of the reason the electronic activity was filtered out, blocked or deleted or redacted. The indication can indicate the type of filter or rule that triggered or caused the removal or redaction.

In some embodiments, the tagging engine 265 can tag the electronic activities with a filter tag based on one or more rules or policies. The filtering engine 270 can then filter out the electronic activities based on the assigned filter tag or cause another system or component to filter out the electronic activity responsive to the filter tag. For example, the tagging engine 265, using technologies such as regular expressions, pattern recognition or NLP, can tag the electronic activity to cause the filtering engine 270 to block ingestion of the electronic activities or perform other filtering in downstream systems.

The system 200 can be configured to provide, via the filtering engine 270, various types of filtering techniques that may be applied to electronic activities during ingestion, during processing of the electronic activities, or when attempting to match electronic activities to record objects of shadow system of records or master system of records.

As described herein, the filtering engine 270 can be configured to apply different types of filtering techniques. As will be described herein, the filtering engine 270 can apply filters based on the content included in electronic activities. Such filters may be referred to as content filters. The filtering engine 270 can also apply logic based filters based on one or more logic based rules for filtering electronic activities. Such filters may be referred to as logic based filters. In addition, the filtering engine 270 may apply filters to restrict matching of electronic activities to node profiles or restrict matching of electronic activities to one or more record objects of systems of records. Additional details regarding the different types of filters are provided herein.

As described herein, the filtering engine 270 can apply various filtering techniques at a user specific level, a company level, a system level, among others. These filtering techniques can be controlled by users, administrators of a company, administrators of the system 200, among others.

a. Content Based Filtering

The filtering engine 270 can be configured to perform content filtering. Content filtering involves performing one or more actions on an electronic activity based on the content of the electronic activity. In some embodiments, the actions can include restricting ingestion of the electronic activity into the system 200. In some embodiments, the action can include redacting a portion or all of the content included in the electronic activity. In some embodiments, the action can include restricting matching the electronic activity to a node profile or restricting matching the electronic activity to one or more record objects.

As described herein, the tagging engine 265 or electronic activity parser 210 can identify terms, text, content or other information in the body or metadata of the electronic activity. The tagging engine 265 can then apply a rule, policy, logic, machine learning algorithm, or natural language processing techniques to assign one or more tags to the electronic activity based on the identified terms, text, content or other information in the body or metadata of the electronic activity. The tag can include a content filter tag or other type of tag that the filtering engine 270 can use to perform content filtering. In some embodiments, the tagging engine 265 can be configured to apply a tagging policy that uses keywords and NLP to identify portions of electronic activities that satisfy one or more filtering rules. The tagging engine 265 can then tag such electronic activities with appropriate content filter tags that the filtering engine 270 can use to either redact portions of the tagged electronic activity, block the entire tagged electronic activity from being ingested, stored, or otherwise processed. In some embodiments, the filtering engine 270 can be configured to parse electronic activities to determine if the respective electronic activity includes any of one or more predetermined keywords, phrases, regex patterns or content in the electronic activity. Responsive to determining that the electronic activity includes any of one or more predetermined keywords, phrases, regex patterns or content in the electronic activity, the filtering engine 270 can restrict ingestion of the electronic activity into the system 200.

In some embodiments, the tagging engine can tag the electronic activity with a tag indicating that the electronic activity includes sensitive information. In some embodiments, the tagging engine 265 can be configured to assign specific content filter tags based on the type of content detected in the electronic activity. For instance, the tagging engine 265 can assign a social security tag responsive to detecting a social security number (or any other number that matches a regex pattern corresponding to a social security number). In some embodiments, the tagging engine 265 can run one or more algorithms to identify various types of information for which a content filtering rule applies. Accordingly, the tagging engine 265 may determine if the electronic activity includes content that satisfies a content filtering rule, the tagging engine 265 may assign one or more content filter tags to the electronic activity indicating that the electronic activity includes content that may be subject to a content filtering rule. In some such embodiments, the content filter tag can include additional information that the system 200 or the filtering engine 270 can use to determine a basis for why the electronic activity satisfies the content filtering rule.

Based on the type of content filter tag assigned to the electronic activity, the filtering engine 270 can take one or more actions on the electronic activity. In some embodiments, the tagging engine 265 can tag the electronic activity with a content filter tag that the filtering engine 270 can use to determine what action to take on the electronic activity. For instance, in the example of the tagging engine 265 assigning a social security tag responsive to detecting a social security number (or any other number that matches a regex pattern corresponding to a social security number) in the electronic activity, the filtering engine can be configured to parse the electronic activity to identify the content that matches the regex pattern of the social security number and can apply a redaction policy to the electronic activity, causing the filtering engine 270 to redact the number from the electronic activity. It should be appreciated that the filtering engine 270 can redact the content by either obscuring the text with a visual marker, replacing the numbers with text indicating that the content is redacted, or other techniques for redacting text. The system can be configured to determine other types of sensitive information including credit card numbers, bank account numbers, date of births, or other sensitive or confidential information for which the filtering engine 270 may include one more filtering rules.

In some embodiments, the tag can indicate a type of data or field present in the electronic activity, such as a social security number or credit card number, in which case the filtering engine 270 can be configured with a policy to redact out sensitive information from electronic activities or filter out electronic activities tagged as containing credit card numbers or social security numbers or other sensitive or private information or perform any other action.

### B. Tag Based Filtering

As described above, the filtering engine 270 can use one or more content filters or content filtering policies to filter the electronic activity. In some embodiments, the filtering engine 270 can be configured to filter electronic activities based on one or more tags assigned by the tagging engine 265. Some of the tags assigned by the tagging engine 265 can be used to filter electronic activities, either from ingestion by the system 200 or from matching or linking the electronic activity to record objects of one or more systems of record. The tags assigned by the tagging engine 265 can be used for purposes other than filtering, for instance, for updating node profiles, determining connection strengths between nodes, understanding context of electronic activities, among others.

The tagging engine 265 can first tag all electronic activities based on numerous tagging methods as described herein. Thereafter, the filtering engine 270 can determine or choose to filter content out based on one or more tags, and otherwise determine or choose to allow further processing or storage of electronic activity based on other tags. The tagging engine 265 can determine to generate, apply or assign a tag, such as a filter tag, based on a regular expression. A regular expression (or regex or regexp) can include a sequence of characters that define a pattern. Example regular expressions can be configured to detect credit card numbers, social security numbers, license numbers, date of birth or any other combination of words or numbers. The tagging engine 265 can be configured with a regex for a credit card number. For example, a regex for a credit card number can be defined as a sequence of 13 to 16 digits, with specific digits at the start that identify the card issuer. The tagging engine 265 can be configured with predetermined digits of card issuers. The tagging engine 265 can apply the credit card number detection technique to the electronic activity (or metadata thereof) to detect or determine whether the electronic activity contains a credit card number. If the tagging engine 265 determines that the electronic activity contains a credit card number responsive to applying or searching for the credit card regex, the tagging engine 265 can apply a filter tag to cause the filtering engine to filter out the electronic activity or redact out the credit card number.

The tagging engine 265 can determine to apply a filter tag based on predetermined keywords. The keywords can indicate topics, concept or terms. Keywords can include, for example, “Credit Card No.” or “License No.” or “SSN” or “SSID”, etc. Keywords for topics to be filtered out can include, for example, “medical record”, “health record”, “doctor visit”, etc. The system 200 can use a master list of keywords that can be used to form a Global Keyword Based Content Filter that can be applied across all (new and existing) customers or users of the system 200. It should be appreciated that the system 200 can be configured to generate, maintain, use or otherwise access keyword ontology or one or more machine learning models trained on keywords, clusters of text or other documents to build the master list of keywords. The content filter can be global or the content filter can be specific to a customer, user or other category or level. The system 200 can continue to update this global list of keyword based content filter. The system 200 can use filters based on a natural language processing technique to determine or identify synonyms, translations into other languages, or related keywords.

As described herein, a filter or filter tag can be applied or generated for a type of electronic activity. For example, a type of electronic activity can be adding a non-human participant (e.g., a room, device, projector, printer, display, etc.) to an electronic meeting event. The system 200 can use a filter to prevent or block further processing on an electronic activity associated with adding a non-human participant or prevent a non-human participant from being matched or from being created as a new record in the multi-tenant, shadow, or other systems of record.

### C. Logic-Based Filtering

The filtering engine 270 can be configured to perform logic-based filtering in which the filtering engine 270 applies one or more logic-based rules to filter electronic activities. The logic filter can include a set of logic-based rules that can be used to filter electronic activities. The filtering engine 270 can be configured to execute one or more logic filtering policies by identifying structured metadata around an electronic activity or record object, and then blocking the electronic activity or record object from being ingested by the system 200 based on identifying the structured metadata. In some embodiments, the logic-based filtering can apply one or more rules or heuristics to restrict matching an electronic activity to a node profile or to one or more record objects of systems of record. In some embodiments, the logic-based filtering can restrict an electronic activity from being matched to a particular record object if the electronic activity was sent by a bot or is sent to a personal email address (such as a gmail address or a hotmail address, among others).

In one example, the system 200, an administrator of the data source provider or a user of the system 200 can establish a logic-based filter to restrict ingesting electronic activities that satisfy one or more logic-based rules. In this example, the administrator of the data source provider can establish a logic-based filter to restrict ingestion of electronic activities that relate to one or more predetermined federal, state, or local government agencies, for instance, the CIA, NSA or FBI. The administrator can create one or more logic based rules that restrict the system 200 from ingesting the electronic activity into the system 200 if the electronic activity can be matched to an account type field having a value of government, or if the electronic activity is sent from or received by a domain name that matches a contains a domain name that matches any of the predetermined federal, state, or local government agencies, or if the contents of the email include certain predetermined character strings (for instance, CIA, NSA or FBI) or if the system 200 otherwise determines that the electronic activity is in any possible way related to the CIA, NSA or FBI.

### D. Matching Filter

As described above, matching filters can be a type of restriction strategy or restriction policy that can be used to restrict electronic activities from being matched to record objects. These matching filters can be a part of, include or use one or more restriction strategies to prevent electronic activities from being linked to particular record objects or a particular system of record in general. In some embodiments, the matching filter can restrict matching electronic activities to record objects if the matching score between the electronic activity and the record object is less than or equal to a predetermined threshold (e.g., 70%, 60%, 50%, etc.). The matching score can indicate how closely the electronic activity matches the record object.

In some embodiments, users of a system of record can be configured to establish one or more restriction strategies or matching filters to restrict matching electronic activities to certain record objects. The user can be a user associated with the record object. In some embodiments, the user can be an administrator of the system of record and can establish one or more matching filters that can be used to restrict matching. For instance, an administrator can establish a matching filter that restricts matching electronic activities including a credit card number to certain record objects. In some embodiments, the matching filters can include multiple rules, which when satisfied, restrict the electronic activity from being matched to a certain record object. In some embodiments, the matching filters can be used to restrict electronic activities from being matched to record objects even if the record object was selected by one or more matching strategies 1100 and 1104 as described above.

### E. Nlp Based Tags and Filtering

The tagging engine 265 can determine to apply a filter tag, or the filtering engine 270 can determine to perform filtering, based on natural language processing. Natural language processing can refer to parsing metadata associated with the electronic activity to identify a meaning, concept, topic or other higher level concept associated with the metadata. Natural language processing techniques or algorithms can determine whether the electronic activity contains or is regarding a concept that is to be filtered out.

Natural language processing can be used to determine whether an electronic activity is a personal electronic activity or a business electronic activity. The filtering engine 270 can perform or execute the filter to redact out sensitive content, block or prevent further processing of personal electronic activities, and authorize or approve further processing of business related electronic activities. The tagging engine 265 when determining to apply a filter tag, or the filtering engine 270 when determining whether to perform filtering, can determine whether the electronic activity is personal based on a tag from the tagging engine or an email identifier used by the sender or recipient (e.g., a domain of the email address indicating personal use (or typically used for personal use) versus a domain indicating a business use or corresponding to an employer of the sender or recipient). The system 200 (e.g., via electronic activity parser 210, tagging engine 265, or filtering engine 270) can further determine personal or business electronic activities based on keywords, terms, topics, or concepts of the electronic activity (e.g., vacation-related versus order or purchase related). In some cases, the system 200 can determine that an electronic activity was sent using a personal electronic mail address, but the content of the electronic activity was to further a business objective. Thus, the system 200 can initially determine to block the electronic activity, but then determine to authorize or approve the electronic activity, thereby overriding an initial filter layer.

The system 200 can use a natural language processing engine that can be configured to parse text or keywords in different languages and determine synonyms or equivalent concepts across multiple languages. For example, the system 200, using the natural language processing engine, can determine the equivalent of a keyword in English but in Japanese, and, therefore, be configured to perform tagging and filtering in multiple languages. The NLP engine can further expand a keyword into a number of synonyms or related keywords. Thus, even if the list of keywords used for filtering are not comprehensive, the system can still perform robust tagging and filtering.

### F. Machine Learning Based Filtering

The system 200 can use machine learning to determine whether to filter an electronic activity. Machine learning can refer to a training set of data that includes metadata for electronic activities that are to be approved or authorized for further processing, as well as metadata for electronic activities that are to be filtered out based on both structured data and also on vectorized content of the communications. For example, if words in the electronic activities are converted into vectors with word2vec or similar technology, a machine learning model can be trained based on the content of the electronic activities alongside (not mutually exclusive) with natural language processing systems. The machine learning based filter can automatically establish, based on the training set, features, weights or other criteria that indicate whether or how an electronic activity should be tagged. In some embodiments, the system 200 can then determine if the electronic activity should be approved or filtered out based on the one or more tags. Thus, by using a machine learning technique, the system 200 can automatically determine features, weights or criteria to detect in metadata of the electronic activity to determine whether to tag and then filter out or authorize the electronic activity for further processing.

### G. Filtering Based on Bot Detection

The machine learning filtering technique can include bot detection. The system 200 (e.g., electronic activity parser 210, tagging engine 265 or filtering engine 270) can use or be configured with a bot detection machine learning algorithm to detect whether the electronic activity was sent by a bot —such as an automatic electronic activity generator. If the electronic activity was transmitted by a bot, the tagging engine 265 or the filtering engine 270 can tag the electronic activity with a tag indicating that the electronic activity was generated by a bot. In some embodiments, the filtering engine 270 can remove or prevent further processing or storage of the electronic activity (e.g., responsive to the tagging engine 265 tagging the electronic activity with a filter tag or a tag indicating that the electronic activity was transmitted by a bot). The system 200 can leverage or use the node graph to automate populating a blacklist of email addresses or other unique identifiers associated with bots through bot detection for syncing. For example, if an electronic activity is from a bot, that information may not be matched, linked or synced to a system of record. In some embodiments, the system 200 can detect bots based on the node graph since the node graph can indicate that a node of the sender bot is associated with edges of interactions between other nodes that indicate heavy one-way interactions across a large number of nodes. Thus, by measuring or detecting edge connection strength between the sender bot node and other nodes, or in some embodiments, comparing the number of inbound interactions (received electronic activities) and outbound interactions (transmitted electronic activities) between the sender bot node and other nodes, the system can classify non-human participants. For instance, non-human participants, such as no-reply@example.com generally transmit emails to a large number of other nodes but only receive a much smaller number of emails from other nodes, thereby allowing the system 200 to classify the email no-reply@example.com as a bot. The system 200 can use or apply similar techniques to detect and classify other types of non-human communication patterns and activity participants. For example, conference room email addresses only get added to meetings but never send or receive regular (non-meeting invite) emails and thus can be classified as Conference Room bots. Furthermore, the system 200 can identify a bot by parsing the email address or name associated with the email address. For instance, the bot detection algorithm can detect that an email address including “no-reply” is associated with a bot. In some embodiments, the bot detection algorithm can parse an electronic activity and determine, using NLP, that the email address associated with the sender is a bot based on language indicating not to send a reply to the electronic activity.

### H. Using Feedback to Improve Filtering Techniques

The system 200 can tune or improve the machine learning techniques based on feedback. For example, upon applying the machine learning techniques to electronic activities, the system 200 can provide the filter decision to an administrator or other user of system 200. The user can input whether the filter decision was correct or incorrect. If the filter decision was correct, the machine learning filter can maintain the weights or rules used to make the filter decision, or increase weights used to make the filter decision. If the filter decision was incorrect, the system 200 can modify the features, weights or criteria in an attempt to correct the filter decision. Similarly, the system 200 can use user input to modify features, weights, or criteria for other types of tagging or filtering, including, for example, natural language processing, rules, linking, or other logic flows that can be improved, enhanced or otherwise benefit from user input. In some embodiments, the machine can be configured to update the weights based on feedback without any intervention of a user or administrator.

### I. Global Pattern Based Process Filter

Since end users can send/receive sensitive information from various systems (e.g., Human Resource systems, payroll systems, benefits systems, applicant tracking systems, recruiting systems, medical bill payment systems, phone bill payment systems, utility bill payment systems, banking or financial institutions, ride sharing systems, etc.) that could include highly sensitive information like payroll, benefits, hiring/termination letters, feedback on hiring a candidate or a cell phone bill which includes call details, the filtering engine 270 can block or prevent such electronic activities from being further processed or ingested by one or more components of the node graph generation system 200 or electronic activity linking engine 250 by maintaining a Global Pattern Based Process Filter which is applicable for all nodes. This can include a pattern based process filter that can identify rules to detect automated emails based on a ‘from’ and other fields; trigger a job to generate an automated systems blacklist; obtain a global blacklist from a storage or database; and apply a global pattern based process to filter out automated system electronic activity. Patterns can also be based on or applied to different fields or aspects of electronic activities, such as other data in an electronic message, meeting or calendar entry, telephone transcript, etc.

To generate such filters, the tagging engine 265 or filtering engine 270 can use bot detection techniques to identify bots automatically and blacklist the identified bots. Further, and in some embodiments, the tagging engine 265 can use natural language processing or machine learning techniques to automatically assess sensitivity of data from a new sender. For example, if a new source starts sending emails to multiple users, and greater than a threshold percentage (e.g., 70%, 80%, 90% or some other percentage) of the emails contain sensitive or confidential information (e.g., social security numbers), then the system 200 can automatically generate and apply a global filter to automatically blacklist this source.

### J. Hierarchy of Filtering Rules

The system 200 (e.g., via electronic activity parser 210, tagging engine 265 or filtering engine 270) can apply one or more layers of filters. The system 200 can apply the one or more layers of filters in parallel or serially. The system 200 can select one or more layers of filters to apply to an electronic activity based on a policy, rule or other logic. Layers of filters can refer to or include different types of filters or different configurations for filters. Layers of filters can refer to or include different type of filter controls or thresholds. Layers of filters can correspond to a hierarchy. For example, a first filter layer can include filtering policies, rules or logic established, based on or customized for a node in the node graph (e.g., a member node, an employee node, a user node, or an individual node). A second filter layer can include filtering policies, rules or logic established, based on or customized for an account. The account can refer to buyer account established by a seller for the buyer. A third filter layer can include filtering policies, rules or logic established, based on or customized for an organization. The organization can refer to or include a buyer organization, such as a company. A fourth filter layer can include filtering policies, rules or logic established by governmental agencies. A fifth filter layer (or master filter layer) can include filtering policies, rules or logic established, based on or customized for an administrator or provider of the node graph generation system 200 or electronic activity linking engine 250. Different entities can establish various types of filters with various thresholds, controls, rules or policies.

As described below, the system 200 can be configured to apply different types of filtering policies. Each of the filtering policies outlined below can correspond to one of the filter layers described above.

### K. Entity-Defined Filtering Policies

The system 200 can select one or more filters to apply to an electronic activity or all electronic activity ingested. The system 200 can select the one or more filters based on the metadata associated with the electronic activity. The system 200 can select the one or more filters to apply based on filtering rules defined for an account, a user, a group of users within an enterprise, an enterprise, or the system 200.

i. Account-Specific Filtering Policies

The filtering engine 270 can maintain account-specific filtering policies that include one or more rules defined for one or more accounts. For instance, the filtering engine 270 can be configured to apply filters to emails either transmitted by or received by a specific account, such as an email account. In some embodiments, the account-specific filtering policy can include one or more rules to apply one or more content filters, logic-based filters or matching filters on electronic activities corresponding to the specific account. For instance, the filtering engine 270 can apply an account-specific filtering policy to an account, such as an email address corresponding to a bot. In one example, the account-specific filtering policy can include a rule to restrict matching any emails transmitted by the account to a record object of a system of record. The account-specific filtering policy can be defined by a user, an administrator of the enterprise, or an administrator of the system 200.

ii. User-Specific Filtering Policies

The filtering engine 270 can maintain user-specific filtering policies that include one or more rules defined for specific users. For instance, the filtering engine 270 can be configured to apply filters to emails either transmitted by or received by a specific user. In some embodiments, the user-specific filtering policy can include one or more rules to apply one or more content filters, logic-based filters or matching filters on electronic activities corresponding to the specific user. For instance, the filtering engine 270 can apply a user-specific filtering policy that includes restricting certain electronic activities sent by the user from being linked to one or more systems of record or to the node profile of the user. For instance, the user may define a rule to restrict any emails between the user and their lawyer from being linked to one or more systems of record or to the node profile of the user. In another example, the user may define a rule to restrict emails sent to the user's spouse at a given company to be linked to record objects of the company. The user-specific filtering policy of a user can be defined by a user, an administrator of the enterprise, or an administrator of the system 200.

iii. Group-Specific Filtering Policies

The filtering engine 270 can maintain group-specific filtering policies that include one or more rules defined for specific groups of users. For instance, the filtering engine 270 can be configured to apply filters to emails either transmitted by or received by users defined within a specific group of users. In some embodiments, the group-specific filtering policy can include one or more rules to apply one or more content filters, logic-based filters or matching filters on electronic activities corresponding to the specific group of users. For instance, the filtering engine 270 can apply a group-specific filtering policy that includes restricting certain electronic activities sent by a user of the group from being linked to one or more systems of record or to the node profile of the user. For instance, a user of the group or an administrator of the enterprise associated with the group or the system 200 may define a rule to redact text included in any emails between one or more users of the group before storing the electronic activity in the system 200 or any record object with which the electronic activity is matched. The group-specific filtering policy of a user can be defined by a user, an administrator of the enterprise, or an administrator of the system 200.

iv. Enterprise-Specific Filtering Policies

The system 200 can select the one or more filters to apply based on enterprise-specific filtering policies. For example, an administrator of a first enterprise or customer of the system 200 can indicate to remove electronic activity accessed via a mail server of the first customer having metadata that matches a regex for a credit card number, whereas an administrator of a second enterprise or customer of the system 200 can indicate to remove electronic activity accessed via a mail server of the second customer having metadata that matches a keyword or regex pattern for a credit card number. In this example, the system 200 applies enterprise-level filtering rules that may be defined by specific enterprises on how to process electronic activities received from their electronic communications servers. The enterprise-specific filtering policy of a user can be defined by an administrator of the enterprise or the system 200.

v. System Defined Filtering Policies

The filtering engine 270 can maintain system-specific filtering policies that include one or more rules defined for the system 200. For instance, the filtering engine 270 can be configured to apply filters to all electronic activities processed by the system 200. For instance, the system-specific filtering policy can include one or more rules to apply one or more content filters, logic-based filters or matching filters on electronic activities accessible by or ingested by the system 200. For instance, the filtering engine 270 can apply a system-specific filtering policy that includes tagging all electronic activities that include numbers matching a credit card regex pattern with a credit card tag indicating that the electronic activity includes a credit card. The system 200 can then be configured to determine if the filtering engine 270 is to take any additional actions on the electronic activity with the credit card tag, for example, redacting the credit card information, restricting matching the electronic activity to record objects, among others. The system-specific filtering policy can be defined by an administrator of the system 200.

### L. Sensitive Information Filter

The system 200 can determine to filter out an electronic activity responsive to detecting sensitive information or data in or associated with the electronic activity. For example, upon detecting or identifying a social security number or a financial account number in the electronic activity, or a tag indicating sensitive information, the system 200 can filter out the electronic activity. This is an example of one type of content filtering.

### M. Source Based Filtering

The system 200 can select the filter to apply based on who is a sender or recipient of the electronic activity or a source of the electronic activity (for example, whose mail server the electronic activity came from). In some cases, the system 200 can select one or more filters to apply or apply all filters that are configured or compatible with the electronic activity. This is similar to the user-specific filtering policy described above. In one example, the data source provider of a system of record can establish a rule that causes the system 200 to restrict any emails coming from a craigslist.org domain from being matched to any record object of the system of record.

### N. Previous Communication Activity Filter

The system 200 can filter out the electronic activity if there have been no previous electronic activities between the sender and recipient of the electronic activity. However, the system 200 can authorize or approve the electronic activity if electronic activities have occurred between a node in close proximity in the node graph to the sender node or recipient node. In some embodiments, two nodes may be in close proximity in the node graph if they have a connection strength above a predetermined threshold. In some embodiments, two nodes may be in close proximity in the node graph if they have either exchanged electronic activity with each other or with a predetermined number of connection nodes in common. Thus, the system 200 can determine to approve or filter out electronic activities based on the extent to which prior electronic activities between the sender and recipient have occurred (e.g., a metric associated with one or more prior activities satisfying a threshold).

### O. Geographic Location Based Filters

The system 200 can select filters based on a geographic location inferred for a node associated with the electronic activity. In some embodiments, a geographic location can be inferred for a node based on detecting a time zone based on timestamps of electronic activities transmitted by the sender node. In some embodiments, the filtering engine 270 can be configured to apply one or more filtering rules based on a geographic location of the sender or recipient of the electronic activity. In some embodiments, geographic location based filtering may be applied in conjunction with the previous communication activity filter or other types of filters. For instance, the system 200 can be configured to restrict matching electronic activities sent to a first user from a second user if the number of communications between the two users is less than a certain threshold and the first user is in a particular geographic region, for instance, Massachusetts.

In some embodiments, the system 200 can authorize or approve further processing or storage of electronic activities if a user has consented to the further processing or storage of such electronic activities. The system 200 can authorize or approve further processing or storage of electronic activities if, based on the overall volume or nature of communications, as well as keywords or context, the metadata associated with the electronic activity indicates an opportunity, contract, or other relationship. The system 200 can determine, using the overall volume, context and nature of electronic activities, (which can be determined using keywords, machine learning or natural language processing), whether the electronic activity is indicative of a legitimate business interaction (e.g., amount of time spent on electronic activities, number of electronic activities, roles, or type of electronic activity such as in-person, video conference, web conference, telephone call). Responsive to this determination, the system 200 can authorize or approve the electronic activity for further processing, or conversely delete, remove or block further processing or storage of the electronic activity if the electronic activity is not indicative of a legitimate business interaction.

Using the filters, the system 200 can determine which electronic activities are to be processed by the system or added, stored or linked to nodes in a node graph or a system of record or one or more systems of record. As described herein, the filtering techniques can be used to prevent sensitive or private electronic activities from being linked or stored in a system of record. The filter can work by (1) completely filtering out the electronic activity, or (2) filtering out/blocking ingestions of the content of the electronic activity, while the electronic activity itself, without the content body, is synced and a graph edge between the sender and recipient of the electronic activity is created, or (3) by redacting out sensitive parts of the electronic activity.

In some cases, the filtering engine 270 can be configured to apply one or more filtering policies to one or more systems of record to scrub or remove data from the systems of record that satisfy the one or more filtering policies. For example, a system of record of an enterprise can be pruned using one or more enterprise-specific filters to remove electronic activities or other values or data from the system of record of the enterprise that satisfy the one or more enterprise-specific filters. For instance, an administrator of an enterprise can establish a new filtering policy to redact social security numbers from any electronic activities that include social security numbers and that are also matched to the systems of records of the enterprise. The filtering engine 270 can be configured to evaluate, responsive to the new filtering policy, each electronic activity previously matched to the systems of record of the enterprise to identify if the electronic activity includes a social security number and if so, redact the social security number from the electronic activity.

It should be appreciated that the filtering engine 270 can be configured to apply one or more filtering policies defined by a user of the system, an administrator of the enterprise or an administrator of the system 200 to prune one or more systems of record, a shadow system of record, or a master system of record.

In some embodiments, the system 200 can identify electronic activities or record objects having personal email domains. The system 200 can maintain a static list of all personal email domains to determine which domains are personal and which domains are not personal. However, to prevent personal electronic activity from being matched or synced to the system of record, the filtering engine 270 can allow linking and syncing based on the domain name.

### 8. Systems and Methods for Threshold-Based Data Management

At least one aspect of the present disclosure is directed to systems and methods for threshold-based data management. The system 200, such as the tagging engine 265, filtering engine 270, electronic activity parser 210 or other component or module, can analyze a data source provider's system of record to identify with which contacts or nodes, employees or users associated with the data source provider have sufficient activity above a predetermined level or threshold. Responsive to the system 200 determining that the level of activity between a user associated with the data source provider and a contact or node is equal to or greater than (i.e. satisfies) a predetermined threshold, the system 200 can authorize, allow, or approve for storage an electronic activity between the user associated with the data source provider and the contact or node. For example, if the user associated with the data source provider has communicated with the contact or node before or had a certain number of communications or certain number of communications of a certain type or certain context, then the system 200 can determine that level of interaction satisfies a threshold and proceed to store the electronic activity or metadata or other information associated with the electronic activity or the node or contact. In this way, cold emails, unsolicited emails or other types of electronic activities that do not warrant being linked to the system of record of the data source provider can be restricted from being linked to the system of record of the data source provider.

The system 200 can detect, using keywords, machine learning or natural language processing, whether the electronic activity is indicative of a legitimate business interaction based on the volume, nature, content or context of the electronic activity or based on the number of electronic activities transmitted between the user associated with the data source provider and the contact. For example, the system 200 can detect a legitimate business interaction based on the amount of time the contact or user associated with the data source provider spent on electronic activities. In some embodiments, the system 200 can detect a legitimate business interaction based on a number of electronic activities, roles, direction of the electronic activity such as inbound or outbound, or type of electronic activity such as in-person, video conference, web conference, or telephone call. Responsive to determining whether the electronic activity is indicative of a legitimate business interest, the system 200 can authorize or approve the electronic activity for further processing, or otherwise delete, remove or block further processing or storage of the electronic activity.

In some embodiments, the system 200 can leverage a node graph to determine the level of activity between an employee associated with the system of record and a contact. The system 200 can determine the level of activity based on the number of electronic activities transmitted by the employee to the contact, the number of electronic activities transmitted by the contact to the employee, or the type of electronic activities being transmitted, information associated with the electronic activities (e.g., calendar invite for a teleconference or in-person meeting, blast email, etc.). In some embodiments, the system 200 can authorize or approve the electronic activity if electronic activities have occurred between a node in close proximity in the node graph to the sender node or recipient node. In some embodiments, two nodes may be in close proximity in the node graph if they have a connection strength above a predetermined threshold. In some embodiments, two nodes may be in close proximity in the node graph if they have either exchanged electronic activities with each other or with a predetermined number of common nodes. Thus, the system 200 can determine to approve or filter out electronic activities based on whether prior electronic activities between the sender and recipient have occurred, or the extent to which prior electronic activities between the sender and recipient have occurred (e.g., a metric associated with one or more prior activities satisfying a threshold).

In some embodiments, the system 200 can scan or crawl the content of electronic activities received from a contact or node to detect proof of consent or other indication of interest (e.g., detecting a type of intent) using natural language processing. If the system 200 determines, based on the content of the electronic activities, that the contact gives permission to store data associated with the contact, then the system 200 can proceed to store the data associated with the contact.

As described above, the system 200 can be configured to determine if electronic activities are personal or business, and through such electronic activities between two nodes, classifying the relationship between the two nodes as either personal or business (and then tagging such relationship as personal/business and then other parts of system 200 can use such tags to perform one or more functions or actions, including filtering, matching, among others).

In some embodiments, certain companies may establish one or more rules to limit the initiation of communications from an employee of the company to other nodes or people outside of the company. The system 200 can be configured to assist such companies by identifying contacts of the employees that the employee may be allowed to contact based on the employee's previous electronic activities with other nodes or people or based on certain types of introductions.

To do so, the system can be configured to detect business or professional introductions from electronic activities. In some embodiments, the system can be configured to determine if an electronic activity, using NLP or other techniques, whether an electronic activity can be tagged with an “introductory” tag. The tagging engine 265 can determine if an electronic activity or a sequence of electronic activities should be tagged with an introductory tag responsive to determining that the context of the electronic activity is one that relates to an introduction. The system 200 can then determine the participants of the electronic activity and create a tag or an indication in their respective node profiles or elsewhere in the system 200 that the participants have been introduced and qualify as contacts. Upon qualifying the participant as a contact of the other participant, if the other participant is an employee of a company that employs rules limiting the types of people the employee can contact, the system 200 can identify the participant as a person the employee can contact. In some embodiments in which the system 200 can update one or more systems of the company, the system can provide an indication to the system that the employee is authorized to contact that person now that the person is a contact of the employee.

### 9. Systems and Methods for Maintaining an Electronic Activity Derived Member Node Network

At least one aspect of the present disclosure is directed to systems and methods for maintaining an electronic activity derived member node network. For example, a member node profile for a member node in a node graph can include information such as first name, last name, company name, phone number, email address, and job title, among others. However, it may be challenging to accurately and efficiently populate fields in a member node profile due to large number of member nodes who may change companies, get promotions, change names (for instance via marriage, or change locations, among others. Furthermore, permitting self-reporting on information in member node profiles by member nodes can result in erroneous data values, improper data values, or otherwise undesired data values. Having erroneous data values in a member node profile that are unsubstantiated by data points serving as evidence to a value of a node profile can cause downstream components or functions that perform processing using the member node profiles to malfunction or generate faulty outputs.

Thus, systems and methods of the present disclosure can generate an electronic activity derived member node network that includes member node profiles for member nodes that are generated or updated based on electronic activity processed by the system. By generating the member node profiles for the member nodes using electronic activities or systems of records and a statistical analysis, the system 200 can update member node profiles using electronic activities, record objects of systems of record and other data points as described above. The system 200 can generate or update member node profiles using data included in systems of record of data source providers, and validate values included in the member node profile using electronic activities and record objects and a statistical analysis.

Furthermore, the node graph generation system 200 can further establish links, connections or relationships between member node profiles based on electronic activities exchanged between them or other electronic activities processed by the node graph generation system 200. These established links, connections or relationships and the corresponding node profiles form the node graph generated by the node graph generation system 200.

By generating the member node profiles and the corresponding node graph by processing electronic activities traversing through or being processed by the node graph generation system 200 and accessing information included in one or more systems of record, the node graph generation system 200 can generate the member node profiles using a statistics-driven analytics process based on the electronic activities, thereby improving upon existing node graphs that are generated based on self-reported information by users. Such existing node graphs are not dynamically updated automatically based on electronic activities and may include information that is inaccurate or not vetted as the information is self-reported with no or little verification.

Furthermore, as the node graph generated by the system 200 is generated in part using electronic activities that are continually being generated and transmitted, the node graph can remain current and up to date without requiring any self-reporting on the part of the nodes associated with the node profiles. Furthermore, given that the node graph is updated as more electronic activities are generated, the system can, using certain parameters, such as dates, be able to determine a status of the node graph at any particular point in time. This is because the node graph is generated, in part, based on electronic activities that are time-stamped and as such, electronic activities that occur before the particular point in time can be used to determine the status of the node graph while electronic activities occurring after the particular point in time can be discarded from the analysis relating to determining the status of the node graph (and individual node profiles) at the particular point in time.

To generate the member node profiles, the node graph generation system 200, or components thereof such as the node profile manager 220, can receive electronic activities including any information related to the electronic activities. The node graph generation system 200 can maintain an array of a time series data set of data points or sources for every value of every field, parameter, or attribute of every node. As also described above with respect to the node profile manager 220, the node graph generation system 200 can associate node profiles of the node graph to electronic activities, or update node profiles that form the node graph based on updates detected by the system 200 responsive to parsing the electronic activities. The node graph generation system 200 can automatically detect potential changes to fields of node profiles of the node graph based on patterns in the electronic activities, and then determine to trigger an update to the node graph. The node graph generation system 200 can sync with one or more systems of records to determine additional information that can be used to update one or more member node profiles. As described herein, updating a node profile does not necessarily mean changing a value of a node profile. In some embodiments, updating the node profile can include adding additional data points to a value data structure to increase or adjust a confidence score of a value corresponding to the value data structure. In some embodiments, the data points can be electronic activities. In some embodiments, the data points can be values determined from record objects of one or more master systems of record. In some embodiments, the system 200 can receive information from record objects of one or more systems of record and use the information to create new node profiles or update existing node profiles by adding data points to support values of fields of such node profiles.

Each node profile can include values that are based on one or more data points. The system is configured to determine, for a particular time, a state of any node profile. The state of the node profile at any given time can be a representation of the node profile using electronic activities and systems of record data that occurred prior to the given time. For instance, the system is configured to output a job title of a given node at a particular date, for example, Dec. 2, 2017. The system can do so by discarding any electronic activity generated after Dec. 2, 2017 and any data from a system of record that was modified after Dec. 2, 2017.

Similarly, the system can be configured to detect changes to a node profile and generate a timeline of changes to values of fields of the node profile. For instance, the system can be configured to detect that a node has changed jobs or gets a new title, among others, based on monitoring electronic activities accessible to the system. For instance, the system can determine that a node has changed jobs if the system detects bounce back activity from the email address of the node and also detects that a person with the same name, phone number in the email signature (or other values) as the node is sending emails from a new email address, perhaps, around the same time that the system detects bounce back email activity from the email address of the node. Similarly, the system can detect a change in the job title based on a change in a signature of the node. The system can then identify a date that the signature was first changed to reflect the new title and mark that date as a date of the title change. In this way, the system can detect when users or nodes get promotions, demotions, join new divisions, leave jobs, start new jobs, among others.

In some embodiments, the system 200 can be configured to provide companies access to data collected, generated and managed by the system 200. The data managed by the system 200 can be used to provide insights to the companies, improve the accuracy of data maintained in one or more systems of record of the companies, among others. In some embodiments, the companies that receive access to the data managed by the system 200 can provide access to data maintained by one or more systems of record of the company as well as electronic communications servers (for example, email servers, messaging servers, among others) of the company, phone servers of the company, as well as other data sources maintained or under the control of the company.

Upon a company providing access to the servers storing data of the company to the system 200, the system 200 can be configured to establish one or more communication interfaces with the one or more servers storing the company's data. The servers storing the company's data can include the email servers, messaging servers, the systems of record servers, among others. Upon establishing communication interfaces with these servers, the system 200 can be configured to receive data from each of the servers storing the company's data. The system 200 can ingest the data and process it as described with respect to FIG. 3 and others.

In some embodiments, the system 200 can receive a large number of electronic activities from the electronic communication servers storing electronic activities of the company. These electronic activities can include all electronic activities accessible by the electronic communication servers. Some of the electronic activities received can be emails that were sent many years ago. However, such electronic activities can still be processed by the system 200 even though electronic activities from other electronic communication servers that were generated more recently have previously been processed by the system 200.

Similarly, the system 200 can receive data from one or more systems of record of the company. The systems of record can include record objects that include values of fields. The system 200 can be configured to ingest the data from these record objects of the systems of record and process the data included in the record objects.

The system 200 can be configured to process these electronic activities and record objects by updating one or more node profiles maintained by the system 200 or generating new node profiles responsive to determining that certain electronic activity or record objects do not match any existing node profile with a certain minimum level of confidence. The system can be configured to determine, for each electronic activity ingested by the system 200, whether the electronic activity can be used as evidence to support any value of a field of any existing node profile maintained by the system 200. The system can do so by attempting to match the electronic activity to node profiles of the system 200. Responsive to identifying a node profile with which to match the electronic activity, the system can add the electronic activity as a data point to a value of the field of the node profile that was used to match the node profile with the electronic activity. Similarly, the system can match record objects to node profiles by matching values of node profiles to values of existing node profiles. Once a node profile is matched with an electronic activity or record object, the system can determine if there are any values included in the electronic activity or record object that does not previously exist in the node profile. If so, the system can add a value to a corresponding field of the node profile and add the electronic activity or record object as a data point supporting the added value.

It should be appreciated that the system 200 can be configured to ingest and process each and every electronic activity maintained by the electronic communication servers under the control or direction of the company as well as each and every record object maintained in one or more systems of record of the company. As such, a large amount of electronic activity and record objects are processed and can be used to update existing node profiles maintained by the system 200 or generate new node profiles for the system 200. As additional companies share access to their data with the system 200 and the system 200 processes the data, the node profiles maintained by the system 200 will be further enriched and the data included in the node profiles will be more accurate. Moreover, data that is less accurate will have lower confidence scores while data that is more accurate will have higher confidence scores as there will be more data points that will be contributing towards the confidence score of the correct values. In this way, the node profiles will become more accurate. As a result, as more companies are on boarded and share access to their data with the system 200, the node graph generated from the node profiles will also become more accurate further increasing the accuracy of the system and each of the node profiles and corresponding node graph. For example, the node graph generation system 200 can detect a change in an electronic mail address status responsive to an electronic message bouncing back due to the message being undeliverable or otherwise not deliverable to the sent address, or having an automated “no longer with company” auto-responder. The node graph generation system 200 can further detect information from the electronic activities or the one or more systems of record with which the node graph generation system 200 (or electronic activity linking engine 250) interacts in order to obtain, infer or determine additional information that can be added to the member node profile. By parsing data from a bounce back electronic activity or auto-responder generated electronic activity, the system 200 can determine various pieces of information. For example, by applying natural language processing to auto-responder generated electronic activities, the system can detect different events corresponding to a node profile associated with the email address for which the auto-responder generated electronic activity was generated. In one example, the autoresponder generated electronic activity can indicate that the person is on a vacation. Such autoresponder generated electronic activities can either mention the word “vacation” or some other synonym or words that may suggest a vacation. The electronic activity can also identify a return date indicating a date when the person will return to the office. The electronic activity can also identify another person (along with an email address, title and phone number, if present) to contact while the person is on vacation. The system can be configured to update the node profile of the person to indicate that the person is out of the office until the return date and further update the node profiles of the person and the other person to indicate a connection or relationship between them. The system can learn from the autoresponder generated electronic activity to determine who else to talk to at the company on a specific matter while the person is on vacation. Moreover, the system can monitor different autoresponder generated electronic activities generated responsive to the same email address to determine other connections of the person if different autoresponder generated electronic activities include different people. Furthermore, the system can use the information from the autoresponder generated electronic activity, for example, the other persons, to determine an organizational structure within the company. For instance, if multiple autoresponder generated electronic activities generated responsive to multiple email addresses of different people identify the same person to contact in their absence, the system may determine that each of the different people report to the same person or are assisted by the same person.

In another example, the electronic activity can be a bounce back electronic activity indicating that the email address is no longer active or the person is no longer with company. Such an electronic activity can be referred to as a soft bounce. In such an example, the system can be configured to determine that the person associated with the email address is no longer at the company by parsing the contents of the electronic activity. In another related example, the electronic activity can be a bounce back electronic activity indicating that the email was not deliverable. In such examples, the system can be determined to apply heuristics to determine a cause for the bounce back by identifying the email that triggered the bounce back activity. If there is no other reason, such as the email size being too big, or if multiple recipients, connected to the system 200 have received similar non-deliverable reports over a period of time, the system can make an assumption that the person has left the company. The system may wait for multiple bounce back electronic activities generated responsive to the email address to confirm that the person has left the company. Upon the system confirming that the person has left the company via natural language processing of a soft-bounce electronic activity or multiple bounce back electronic activities responsive to the same email address, the system can update the node profile to indicate that the user is no longer at the company.

The system can further be configured to identify if any electronic activities generated after the date that the bounce back electronic activity was generated that mention the person's name, city and state (for example, in a signature of the email) or other values of the node profile can be matched to the node profile. The system can eventually determine an electronic activity that matches various values of the node profile and can parse the electronic activity to identify a new email address of the node profile. The system can use the bounce back activity as well as the subsequent electronic activity that matched the node profile to identify various events associated with the person. For example, the system can determine that the person left his previous job before the time of the bounce back activity and started a new job on or before the date of the subsequent electronic activity. In some embodiments, multiple electronic activities need to be processed to confirm if a person has left a company or started at a new company. Furthermore, the system can be configured to update the node profile of the person by adding additional electronic activities including the new email of the person once the system determines that the new email address belongs to the node profile of the person. This information can be used to generate or maintain a job timeline (e.g., start date and end date) of the person and can be used to detect when a user changes jobs for instance, or other information associated with the member node.

In some embodiments, auto-responder electronic activities generated responsive to receiving an email can include additional information that can be parsed to better understand the role of the person to whom the electronic activity was sent including identifying people to contact in the person's absence, when the person will become available if at all, and whether the person is still at the company or not.

For instance, an auto-responder generated electronic activity indicating that a user is on maternity or paternity leave may not include an expected date of return or may identify one or more other people to contact during the person's leave. The system may be configured to detect a maternity or paternity leave related autoresponder generated electronic activity. The system can detect the first time the auto-responder generated electronic activity was generated by analyzing multiple electronic activities matched to the node profile of the person. The system can then determine, based on typical maternity or paternity leaves for the company (by monitoring other people's email activity in similar cases) a likely return date for the person and can update the node profile of the person to reflect that they are on maternity or paternity leave. In other embodiments, the system 200 can determine the return date by parsing the contents of the auto-responder generated electronic activity.

The system 200 as described herein can be configured to parse bounce back and auto-responder generated electronic activities to update node profiles or determine additional information about node profiles. In some embodiments, the system 200 can be configured to establish connections with one or more third-party data sources, for instance, marketing automation or mass mailing systems, to receive additional data from such data sources. In some embodiments, the system 200 can access the data for companies that also provided access to their electronic communication servers and systems of record. The system can then harvest the data related to bounce back activity based on electronic activities sent via or generated by the third-party data sources, such as marketing automation systems, and use the data related to bounce back activity to increase the number of bounce back electronic activities the system 200 ingests or can access, thereby further increasing volume of data and further enriching member and group node profiles and the node graph.

In addition to the examples provided herein, the system can be configured to provide job timeline verification, based on electronic activities. The node graph generation system 200 (e.g., via 200 electronic activity parser 210) can identify a sender and recipient of an electronic activity. As described above, the system 200 is configured to attempt to match the electronic activity to a node profile corresponding to the sender and one or more node profiles corresponding to the respective recipients. In some embodiments, the system is unable to match the electronic activity to a node profile of a sender or a recipient if the system has not previously generated a node profile for the sender or recipient. When the node graph generation system 200 detects a new recipient or sender of the electronic activity, based on an identifier such as an email address, the node graph generation system 200 can create a new member node profile for the new recipient or sender. The node graph generation system 200 can, in some cases, determine, using a de-duplication and identity resolution process, that the new member node profile matches or is the same as a previously generated node profile. The node graph generation system 200 can identify, using one or more parsing or processing techniques, a first name and a last name associated with electronic activity. For example, the node graph generation system 200 can parse an electronic signature in the body of the electronic activity or email to identify a first name, last name, job title, phone number, or other contact or identifying information. The node graph generation system 200 can identify fields that have values that do not change when a person moves from one job to another, such as their first name, last name, personal phone number, or other usernames or identifiers not tied to the job. By identifying information that does not change with the job and information that likely changes with the job (e.g., company email address, work phone number or job title), the node graph generation system 200 can map, match, or link the newly created member node profile with a previously generated or created node profile. The previously generated node profile may have included a different email address, such as an email address with a different domain that may correspond to a previous employer, while for example, the mobile phone number stayed the same. The system can determine, thereafter, that the new electronic activity associated with the new email address corresponds to the same member node, but that the member node has switched or changed jobs.

Accordingly, the system can set or establish an approximate start date in the job timeline responsive to detecting the new email address.

Further, the node graph generation system 200 can establish, set or update the previous job timeline with an end date. The node graph generation system 200 can establish, set or update the previous job timeline with the end date responsive to detecting bounce-back emails to the previous email address or the last communication with the member node profile that went through using their old email address. The node graph generation system 200 can further corroborate the end date based on detecting the start date for the new job based on the new email address having the new domain different from the previous domain.

The node graph generation system 200 can update additional information about the member node profile, such as a new company name, a new company address, a new company phone number, a new email address, a new job title, among others. The node graph generation system 200 (e.g., via node profile manager 220) can detect various pieces of information with which to update the node profile by parsing an electronic signature embedded or included in an electronic activity such as an email sent from the new email address. The node graph generation system 200 can use a statistics-driven analysis technique to determine the new company name, the new company address, the new company phone number, the new job title, among others. For example, if the sender of the electronic activity sends 10 electronic messages to 10 different recipients within a predetermined time interval and using the same electronic signature containing the same company name, company address, company phone number, and job title, then the node graph generation system 200 can be configured to update the node profile of the member node to reflect new values for company name, company address, company phone number, and job title. Furthermore, the confidence score of each of these values can be determined and increased as additional emails are sent and received via the new company email address. In some cases, the confidence score in the job title can be further determined based on the recipients of the electronic activities. The system can be configured to maintain, for a given job title of a person, a mapping of volume or distribution of emails to people having certain titles. For instance, a CEO is more likely to send emails to other CEOs or C level executives than a person having a title of associate. Similarly, a person with a sales related title is likely to send more outbound emails than a person with a title related to Human Resources. As such, the system can be configured to determine a confidence score of the job title based on the contribution scores of data points supporting the value but also based on whether the person's emailing activity matches that of other people with similar titles. Stated in another way, in this example, the node graph generation system 200 can perform job title verification based on evaluating node profiles linked to electronic activities that identify the person's new email address.

The node graph generation system 200 can use the member node profile to maintain an accurate organization chart for a given company. For example, a field in a member node profile can include a “Reports to” field. The node graph generation system 200 can maintain, for each value of the “Reports to” field of a node profile, an array of data points identifying sources that include record objects having the “Reports to” field for the node profile to determine the confidence score of the value. In some embodiments, based on the values of the reports to field of multiple node profiles belonging to the same group node or company, the node graph generation system can maintain an organization chart for the company. In some embodiments, job titles of various node profiles can further be used to determine the organization chart. Furthermore, the organization chart can further be determined based on parsing electronic activities, including but not limited to out of office and other autogenerated electronic activities that may include information identifying links between certain node profiles. Moreover, using effort estimation and analyzing the content of electronic activities exchanged between two nodes, the system can further determine a relationship between the two nodes including predicting a boss-subordinate relationship between nodes.

In some embodiments, the node graph generation system 200 can detect job title changes and use the detected change to reevaluate or update an organization chart. The node graph generation system 200 can utilize master data model to match member nodes in a member node graph to a group node in a group node graph (e.g., a company graph). The node graph generation system 200 can use the member node graph to build, generate or update a group node graph that can include a hierarchy or organizational structure comprised of member nodes from the member node graph. As the node graph generation system 200 detects changes or updates to the member node profile of a member node based on parsing electronic activities and email signatures therein, and determines that a confidence score of a value of a field in the member node profile associated with the detected change warrants updating the member node profile, the node graph generation system 200 can update the value in the corresponding field in the member node profile, as well as update a hierarchical organization or structure in the corresponding group node graph or network.

The node graph generation system 200 can present one or more member node profiles for display. The node graph generation system 200 can present the member node profiles for display via a webpage, website, browser, application, or via other presentation medium. For example, the node graph generation system 200 can present a member node profile for display via a mobile application executing on a client computing device having a display device. In some cases, the node graph generation system 200 can present the member node profile via audio output, such as via a voice interface.

The node graph generation system 200 can be configured to hide or otherwise prevent or block from display one or more fields in the member node profile. The member node, such as the owner of the member node profile, can establish the configuration as to which fields, or values thereof, to hide from display. The node graph generation system 200 can provide access control options via a computing device to a member node or user thereof. The node graph generation system 200 can generate a graphical user interface or other type of user interface to present the access control options, as well as receive selections or modifications to such access control options. Using the access control interface generated and provided by the node graph generation system 200, the user can control which fields are presented for display via the web page, for example. In some cases, the user can control which accounts can access the member node profile of the user, or, on a more granular level, control which account can access which fields or values in the member node profile.

In some cases, the node graph generation system 200 can allow a third-party device to request access or request presentation of a value of a particular field in a member node profile. The node graph generation system 200 can receive the request and forward the request to the member node via an electronic activity. The member node can accept or reject the request. In the event the member node accepts the request for access to the value in the field, the node graph generation system 200 can, automatically and responsive to accepting the request, update the access configuration profile for the member node profile. Thus, the node graph generation system can hide or unhide one or more fields (or values) from one or more third-parties or computing devices based on the preferences of the owner of the member node profile.

### 10. Systems and Methods for Monitoring Performance of Node Profiles

As described herein, the node graph generation system 200 can be configured to ingest and process large amounts of electronic activity that are provided by one or more electronic communications servers storing electronic activities belonging to or associated with one or more enterprises or companies. The system 200 can only ingest and process those electronic activities to which the enterprises or companies provide access. The system 200 can also be configured to ingest and process data from systems of record maintained by one or more servers. Similar to electronic activities, the system 200 can only ingest and process those systems of record to which the enterprises or companies provide access. As described herein, the system 200 can process electronic activities and record objects of systems of record to update node profiles of nodes, link or match electronic activities to record objects of the one or more systems of record accessible to the system 200, determine or predict a stage of a business process, among others.

The node graph generation system 200 can further be configured to process electronic activities and record objects of one or more systems of record of a company to determine insights for the company. For instance, the node graph generation system 200 can provide insights to Company A by processing electronic activities and record objects that Company A has made accessible to the node graph generation system 200. The insights can include metrics at a company level, a department level, a group level, a user level, among others. The insights can identify patterns, behaviors, trends, metrics including performance related metrics at a company level, a department level, a group level, a user level, among others. Additional details relating to the insights are described herein.

The node graph generation system 200 can include a performance module 280 that can be configured to generate performance profiles for a company. In some embodiments, the performance profile can be a performance profile of an employee of the company. In some embodiments, the performance profile can be a performance profile of a department of the company, a group within a department, or individual employees of the company. The performance module 280 can generate the performance profiles using data accessible by the node graph generation system 200. In some embodiments, the performance module 280 can generate the performance profiles using all data including electronic activities and systems of record accessible by the node graph generation system 200 from multiple companies. In some other embodiments, the performance module 280 can generate the performance profiles for a company only using data provided by the company to the node graph generation system 200. In some embodiments, the performance module 280 can be configured to generate certain types of performance profiles for employees, groups, departments of a company that has provided access to the system 200 while generating other types of reports or insights for other node profiles of the system 200 that are not employees of the company.

The performance module 280 can be configured to predict employee success at a company or in a job role. The performance module 280 can, based on an analysis of electronic activities as well as information stored in one or more systems of record, predict the success of the member node. For example, the performance module 280 can generate a performance profile for the member node. The performance profile can be a statistics driven performance profile. The performance profile can be based on electronic activities and information stored in one or more systems of record. For example, the performance profile can be based on a number or amount of electronic activities associated with the member node during a time interval, a type of the electronic activities, the amount of time the member node spends generating or preparing the electronic activities (e.g., amount of time spent writing an email), the recipients of the email, natural language processing of the email, etc.

For example, the node graph generation system 200 (via performance module 280), using job history and performance history reconstructed from an internal member node graph, can generate a performance score, purchasing preference, decision making power, interests or other information for the member node. By syncing information associated with the systems of record and electronic activities with the member node graph, the node graph generation system 200 can generate or extrapolate types of opportunities or features on the public profile.

For example, the node graph generation system 200 can determine that a member node performs medical device sales, the member node's territory is the northeast region, the member node prefers or is more successful when doing in-person sales, the member node prefers or more successful when doing CEO level sales, or an average deal size or amount. To do so, the node graph generation system 200 can parse or featurize information corresponding to tasks or activities (e.g., deals) associated with the member node (e.g., a salesperson or other knowledge worker) that is derived from one or more record objects stored in the one or more systems of record. By parsing or generating features from the record objects, the node graph generation system 200 can update a member node profile to reflect various performance information derived from record objects in one or more systems of record as well from electronic activities. The node graph generation system 200 can generate various outputs derived from record objects in one or more systems of record and electronic activities. Outputs can include a performance score or performance grade indicating how well a member node has performed or may perform in general, at a type of task, in a specific job or under certain circumstances of a job or job environment, as determined by the communications metadata, extracted from the node graph.

For example, the node graph generation system 200 can generate an output corresponding to a performance score or performance grade of a user based on an average seniority of attendees to a meeting initiated, established, conducted or led by the user. The node graph generation system 200 can determine the average seniority of attendees to the meeting established by the user by parsing electronic activities associated with the meeting (e.g., calendar invite or emails) to identify the attendees, and further determining the seniority of the attendees based on a member node profile for the attendees or metadata associated with the electronic activities. The node graph generation system 200 can generate an absolute performance score based on the determined seniority of the attendees. In some cases, the node graph generation system 200 can compare the average seniority of attendees to a meeting established by a first user with the average seniority of attendees to meetings established by other users. The system 200 can be configured to determine or measure the number of communications a user is involved in, the types of communications the user is having (by using NLP and other semantic analysis techniques to determine context of communications), and the roles of the people the user communicates with. These metrics or other metrics can be representative of future success. For instance, the system has been configured to determine that employees who drive meetings with a higher average seniority of attendees are more likely to be successful than employees who drive meetings with a lower average seniority of attendees. As such, the use of the tagging engine 265 and the node profiles to assign tags to meetings indicating roles of attendees, seniority of attendees (such as C×O) can be used by the system to predict or measure employee performance and success.

In some embodiments, the system can be configured to track employee activity behavior. The system can utilize supervised or unsupervised machine learning to determine behaviors that result in future success for the employee. For instance, the system 200 can determine that the number of communications a user is involved in, the types of communications the user is having (by using NLP and other semantic analysis techniques to determine context of communications), and the roles of the people the user communicates with are all behaviors, traits, features, metrics or other signals that can be used to predict future success based on training the system 200 on past or other current employees identified as being successful and other employees identified as unsuccessful.

In some embodiments, the system 200 can generate or maintain, for one or more roles of a company, a standardized performance profile generated based on aggregating performance profiles of a plurality of performance profiles of users in the role previously identified as being successful in the role. The system 200 can compare the user's performance profile generated based on the user's activity behavior to the standardized performance profile to predict a likelihood of success of the user and can further be configured to provide feedback to the user on how to improve their performance based on the comparison.

The system 200 can be configured to generate a performance profile of a user based on the user's role as different roles may perform vastly different functions. Two employees in different roles may both be very successful in their roles but their electronic activity footprints may appear very different. For instance, a successful customer success manager's electronic activity footprint or behavior may have a regular cadence of meetings (in-person or telephonic) with each of their customers. Different customers may require different cadence of meetings but a successful customer success manager may maintain the cadence for each of their customers. For the system to determine how well an employee is performing, the system can be configured to monitor, for each customer, whether the employee is having regular, recurring meetings with the customer that matches the cadence of meetings the employee is supposed to have with the customer. The system can determine this based on analyzing the employee's electronic activities to see if meeting requests are sent within particular time periods and meetings actually occur. As described herein with respect to tagging, the system 200 can confirm whether a meeting happened and this information can be used to determine if the employee is having regular meetings. As such, the node graph generation system 200 can determine a performance of a user based on a cadence of meetings with each of the user's customers. The regularity of the cadence can be based on the number of meetings with customers within a time interval, such as a week, two weeks, month, two months, etc.

Furthermore, the system 200 can be trained or configured to use the cadence of meetings for the user's customers to determine a user's level of engagement with the customers. The user's level of engagement can be used as a signal to quantify a user's performance as an employee as a low level of engagement can predict that the customer may disengage with the company or may look elsewhere to service their needs. The system 200 can maintain a user engagement model for each customer or customer type that is based on one or more parameters or metrics. The user engagement model can be used as a benchmark. The system 200 can then compare the user's level of engagement with the user engagement model to determine if the user's level of engagement is below, the same or above the benchmark. If the user's level of engagement is below the benchmark, the system can notify the user or the company and provide tips to increase the level of engagement to improve customer satisfaction and/or reduce the likelihood that the customer may leave.

In some embodiments, a user's performance can be measured on his electronic activity behavior. For instance, for employees in certain roles, the employee's performance can be based on how quickly the employee responds to emails, how much time the employee spends preparing responses to the emails, as well as various other metrics, parameters or attributes that can be determined from the emails the employee sends. In some embodiments, an employee's response time to emails from a customer can be used as a metric to determine the employee's level of engagement with the customer. The employee's response time to emails from the customer can be compared to the employee's response time to other customers to determine the employee's level of engagement with that customer. Furthermore, the quality of the employee's responses may also provide an indication of the employee's level of engagement. For instance, the system 200 can be configured to determine an amount of time the employee spent drafting the email based on a time estimation model that analyzes the number of words, the choice of words, the time difference between when the email to which the employee is responding to was received and the time the response was sent, among others. In some embodiments, the time estimation model can take into account the titles of the participants of the email.

In another example, the node graph generation system 200 can determine the performance score for a user based on the amount of time it takes to receive a response to a ping or electronic activity transmitted by the user. For example, if the user is a recruiter, the recruiter's performance can be based on how quickly he gets job candidates to respond to their email as well as how many job candidates respond to their email, and how many emails (or follow up emails) on average it takes a job candidate to respond to the recruiter. The system 200 can be configured to determine that recruiters that have lower response times (time it takes the candidate to reply to the recruiter) have a higher performance score than recruiters with higher response times. Furthermore, the system 200 can be configured to determine that recruiters with higher response rates (number of candidates who actually respond) and lower average number of emails it takes to receive an initial response to the email perform better than recruiters with lower response rates and higher average number of emails to receive initial responses. It should be appreciated that the system 200 can be configured to generate such statistics for every user type or node having a certain title and comparing the statistics of such users or nodes to generate a benchmark for various parameters that may be factors that contribute to a performance of a user or node.

In yet another illustrative example, the node graph generation system 200 can determine, from the member node graph profile and one or more record objects in one or more systems of record, that a member node performs deals in the northeast region and that the deals are more likely to close when there a certain number of in-person meetings associated with that deal. The node graph generation system 200, utilizing this performance information, can generate an extrapolation curve to determine how well the member node might perform in the future, or forecast performance of the member node.

The node graph generation system 200 can generate the performance forecast based on historical electronic activities, one or more record objects, and a member node profile. Similarly, the node graph generation system 200 can determine based on a low performance score that the employee is likely to fail or leave the company.

In some cases, the node graph generation system 200 can match a member node with a candidate deal or potential deal or ongoing deal. For example, the node graph generation system 200 can match a representative to the right potential or ongoing deal. The node graph generation system 200 can match the representative to the deal based on a social proximity territory assignment, which can be based on a strength of overall relationships of the representative with a certain type of person or member node (e.g., buyers, such as someone who as the authority to close a deal, at target accounts). The node graph generation system 200 can determine that the more people the representative has a relationship with at a target account, the more likely the representative is to succeed with the target account. As such, the node graph generation system 200 can match the representative with target accounts with which the representative has the most relationships as well as the most of strong relationships with people at the target account that are associated with closing a deal or other successful outcome.

The node graph generation system 200 can match representatives to a target account or deal based on a selling style of the representative. For example, the node graph generation system 200 can determine that a target account or buyer prefers a certain type of selling style, such as primarily face-to-face vs over the phone, or meeting certain people at the target account such as the CEO. The node graph generation system 200 can then identify representatives that sell using these styles or are known to perform well using these styles, and then assign the representative to the corresponding target account. For example, if a representative is determined to perform well when meeting a CEO based on analyzing historical deals, electronic activities or profile information, then the node graph generation system 200 can match the representative with a target account that is associated with successful outcomes when the CEO of the target account meets with the representative.

### 11. Systems and Methods for Providing a Company Cloud

At least one aspect of the present disclosure is directed to systems and methods for providing a company cloud. The company cloud can identify a plurality of companies or enterprises. Each company included in the company cloud can be represented as a company or group node and each group node can include or be linked to one or member node profiles corresponding to people belonging to or affiliated with the company. The company cloud can refer to or include a group node graph or network of group nodes. A group node can be a representation of a company and include fields. Fields can include, for example, a company name, a company phone number, a company address, a unique identifier for the company, a company size, a company location, or other information associated with the company. The group node can further be linked to one or more member node profiles corresponding to people who are either employed by the company or in some embodiments, have some affiliation with the company.

In some embodiments, one or more values of the fields of the group node can be populated based on values of one or more member node profiles belonging to nodes that are employed or affiliated with the company. For example, values of various fields such as company name, phone number, address, among others may be derived from node profiles of its employees. In some embodiments, the values of the fields may be associated with value data structures including entries identifying data points that support the value. Such data points can be data points that support values of fields of member node profiles belonging to employees of the company associated with the group node.

Similar to how member node profiles are generated and updated as described above, the node graph generation system 200 can generate company or group nodes using the same sources of data, namely, electronic activities and data from systems of record.

In some embodiments, the node graph generation system 200 can analyze systems of record of different data source providers (for example, enterprises) to identify multiple account record objects representing the same company. The multiple account record objects representing the same company can be maintained in different systems of record belonging to different enterprises. For instance, multiple companies can maintain an account record object for the company, Acme. A first enterprise can maintain a first account record object for the company Acme. The first account record can include a first value for the field Company Phone Number. A second enterprise can maintain a second account record object for the same company Acme. The second account record object can include a second value for the field Company Address. The node graph generation system 200 or the data source provider network generator 260 can create a group node profile for the company Acme by extracting values from both the first account record object and the second account record object such that the group node profile is richer in information than each of the respective first and second account record objects. In some embodiments, the node graph generation system can further be configured to maintain a master account record object for the company that includes values from each of the account record objects across multiple systems of record such that the master account record object of the system 200 is richer in information than each of the respective account record objects across the multiple systems of record. In the example above, the group node profile and the master account record object can include the first value for the field Company Phone Number and the second value for the field Company Address.

The node graph generation system 200 can add or update values of one or more fields of the group node profile for the first group node. In some embodiments, the first time the node graph generation system 200 detects or identifies an account record object or electronic activity to be associated with a particular group node profile of a company, the node graph generation system 200 can create or establish a group node profile for the company. Thereafter, the node graph generation system 200 can continually amend or update that group node profile with additional information or updated information. Further, as the node graph generation system 200 receives conflicting information for the group node profile from different record objects of different systems of record maintained by different data source providers, the node graph generation system 200 can resolve the conflicts using rules, policies, or a confidence score of a value of an attribute or field of a group node profile, for example.

In some cases, the node graph generation system 200 can determine that different systems of record may have different values for the same field for the same group node profile corresponding to a particular account or company. The node graph generation system 200 can use one or more techniques to determine the correct value for the field for the group node profile, or the most accurate or likely to be correct value for the group node profile. The node graph generation system 200 can use techniques for generating or determining the confidence score of a value of an attribute or field in a group node profile. For example, the node graph generation system 200 via the attribute value confidence scorer 235 can determine a confidence score for each value for each attribute or field in the group node profile based on an array of data points maintained for each value.

By analyzing, parsing or otherwise processing multiple systems of record and electronic activities, the node graph generation system 200 can generate a master group node profile for a company or account that contains one or multiple values for one or more of the fields. Similar to member node profiles, the system 200 can be configured to generate a confidence score for each value of the one or more fields that is based on contribution scores of each of the data points supporting the value as evidence.

As described above with respect to member node profiles, the group node profiles can also be updated as more information is ingested by the system 200. In some embodiments, the system 200 can ingest new electronic activities and data from systems of record and periodically update the member node profiles based on the new data. In some embodiments, the system is configured to ingest and process new data once a day, once a week, among others. In some embodiments, the system can ingest and process new data as new systems of record are made accessible to the system. In some embodiments, the system can be configured to ingest and process new data responsive to a request from a user or an administrator of the system 200. In some embodiments, the system 200 can be configured to update the node graph, which can include both group nodes and member nodes, responsive to ingesting or processing the data. The system 200 can be configured to update tags or associated confidence scores assigned to previously processed electronic activities. Furthermore, the system 200 can be configured to update value data structures of node profiles by removing electronic activities previously assigned to a value but determined responsive to new data that the electronic activities were previously assigned to a particular value or node profile based on insufficient data. In some embodiments, such changes to node profiles can be made responsive to determining that the tag or electronic activity is improperly assigned or classified. It should be appreciated that as more data is ingested by the system 200, certain classifications and tags can be misclassified or assigned but can be corrected by the system based on the new data. As such, the node graph generation system 200 can update the group node profiles on a periodic basis, based on a time interval, responsive to a request, or based on new or updated insights or information that is derived from electronic activity data flowing through the system 200 as a time series dataset.

In an illustrative example, a field in an account level for a First Company can be “Parent Account” field. The parent account field can have a value, linking to the record of “Second Company” because Second Company can be the parent company of the First Company. The node graph generation system 200 can determine that this is a field in an account and then extrapolate that this field denominates a parent company in a complex corporate structure when a Second Company owns a First Company, thereby resulting in the Second Company being named in the “Parent Account” field. The node graph generation system 200 can analyze, for example, 50 different systems of record to identify 50 different account record objects that contain an account for the First Company. The node graph generation system 200 can then determine, for each of the 50 different account record objects, the value of the parent account field. If all 50 of account record objects have the same value in the account field (e.g., Second Company), then the node graph generation system can establish a group node profile for a group node in the master group node graph for the account for the First Company to include the value “Second Company” in the parent account field. The node graph generation system 200 (e.g., via attribute value confidence scorer 235) can use one or more policies, rules, weighting systems, scores or other logic to select a value to use for the account field in the group node profile for the group node profile in the master group node graph. For example, the node graph generation system 200, via attribute value confidence scorer 235, can leverage a time-series calculation of values of this field across multiple systems of record, while taking into account a confidence score and recency of each value of each field, where the more recent values are assigned a higher weight.

The node graph generation system 200 can analyze the systems of record on a period basis or based on some other time interval and detect a change in values in fields. The node graph generation system 200 can, responsive to detecting a change in some or all of the systems of record, update the group node profile. For example, the node graph generation system 200 can update the group node profile responsive to detecting the change in 5 of the 50 account record objects of the 50 systems of record. The node graph generation system 200 (e.g., via attribute value confidence scorer 235) can determine that while only 10% of the account record objects reflect a change in the value, that these 10% of account record objects are reflecting an accurate change (e.g., based on high trust scores of the systems or sources that produced the change, and a recency of the field change) and, therefore, the master group node graph is to be updated. The node graph generation system 200 can determine, for example, that this 10% of systems of record are associated with a relatively high trust score which may cause the attribute value confidence scorer to generate a higher confidence score for values of fields received from such systems of record or other score relative to some or all of the remaining 90% of systems of record. Thus, the node graph generation system 200 can detect a change in company ownership or subsidiary status based on a subset of systems of record and before other systems of record are updated to reflect such ownership or organizational change, thereby reducing latency in updating organizational structure across all systems of record, connected to the system.

### 12. Systems and Methods for Improving Member Node Performance Based on Electronic Activity

At least one aspect is directed to systems and methods for improving member node performance based on electronic activity. The node graph generation system 200, or one or more component thereof, can analyze electronic activities associated with member nodes to generate a member node profile for a member node in a member node graph. The node graph generation system 200 can identify metrics for each member node profile based on the electronic activities. The node graph generation system can correlate the metrics with desired performance outcomes or results, including but not limited to closed sales, recruited candidates, or renewed contracts to identify which metrics are correlated with desired performance outcomes. Based on identifying the desired metrics that result in desired outcomes, the node graph generation system 200 can set one or more goals for member nodes, as well as help track those goals to increase the likelihood that the member node achieves the desired performance outcome, thereby improving the likelihood that the member node achieves the desired performance outcome.

In some embodiments, the node graph system 200 can include a recommendation engine 275.

The node graph system 200 (via recommendation engine 275) can provide a recommendation or set a target goal for a member node. The node graph generation system 200 can, for example, provide these recommendations or target goals to one or more member nodes or one or more group nodes based on historical matching electronic activities to desired performance outcomes. The node graph generation system 200 (or one or more component thereof) can match electronic activities to desired performance outcomes stored or indicated in one or more systems of record.

The node graph generation system 200 can include a performance module designed and constructed to determine a performance metric or performance level of a member node based on electronic activities. To generate a recommendation, the node graph generation system 200 (via a performance module 280 and recommendation engine 275) can identify member node performance as compared to a member node's past performance or as compared to the performance of other member nodes that have a similar role or otherwise share similar characteristics. The node graph generation system 200 (e.g., via a member node performance module) can determine a performance of a member node. For example, the node graph generation system 200 can identify electronic activities associated with multiple member nodes that are linked to a group node in a group node graph. The node graph generation system 200 can then identify a system of record associated with the group node. The system of record can include account record objects, lead record objects, opportunity record objects, deal record objects or other types of record objects. The system of record can include stages for any business process, such as opportunities with stages, recruiting of candidate with interview stages, renewing contract with renewal stages, etc. In an illustrative example, an opportunity record object can include multiple sequential stages for the opportunity, such as a first stage, second stage, third stage, and a fourth stage, where the first stage indicates an initial stage and the fourth stage indicates a final or completion stage for the opportunity. The node graph generation system 200 can correlate electronic activities with the opportunity record objects as well as the stages of the opportunity record objects. The node graph generation system 200 can determine metrics based on electronic activities that are associated with an opportunity advancing stages or not advancing stages. For example, the node graph generation system 200 can correlate that, on average: 5 emails and 1 in-person meeting occurred in a time interval for an opportunity before it moved from a first stage to a second stage; 10 emails and 2 in-person meetings occurred during a time interval for an opportunity to move from a second stage to a third stage; 15 emails and 3 in-person meetings occurred during a time interval for an opportunity to move from a third stage to a fourth stage; and 20 emails and 4 in-person meetings occurred during a time interval for an opportunity to move from a third stage to a fourth or final stage. By determining metrics that are correlated with advancing an opportunity from one stage to another based on electronic activities correlated with stages in opportunity record objects stored in a system of record, the node graph generation system 200 (or component or module thereof) can predict or forecast metrics that, when met, are likely to result in the desired performance outcome. The node graph generation system 200 can determine which metrics of electronic activities have the highest correlation to successful outcomes in order to generate goals.

For example, when a member node enters a first stage of a process described in a system of record (e.g., a first stage of an opportunity, recruiting process, contract renewal, etc.), the node graph generation system 200 can identify, for a similar opportunity and a similar member node, the metrics that, on average, likely resulted in a desired performance outcome of advancing from the first stage to a second stage. The node graph generation system 200 can further provide an indication of these metrics to the member node as a goal or target metrics to improve the likelihood that the member node advances from the first stage to the second stage. The node graph generation system 200 can further provide metrics estimated to advance from each stage to the final stage. The node graph generation system 200 can generate the estimate by benchmarking across member nodes in similar roles working on similar processes in order to identify the desired performance outcomes and metrics associated with such desired performance outcome. For example, the benchmarking process can include identifying member nodes that conduct interviews in a recruiting process to identify metrics associated with candidates that accepted an offer to join a company in order to provide an estimate of a metric that might result in a desired outcome. An example metric for this example can include a response time or response quality associated with emails between the interviewer and the candidate before or after the interview. Other example metrics can include the duration of the interview, whether the interview was face-to-face or telephonic, or whether the interviewer or candidate was late to the scheduled interview based on natural language processing of the correspondence between the candidate and the interviewer.

In another example, the node graph generation system 200 can identify member nodes linked to a group node that perform well or have desired performance outcomes. The node graph generation system 200 (e.g., via recommendation engine 275) can identify a temporal aspect to the metrics associated with the member node. The node graph generation system 200 can determine when member node first joined the group node or was first linked to the group node (e.g., a job start date or beginning date), and how the member node's performance and behavior metrics evolved over time. This initial time interval can be referred to as a ramp-up period (e.g., when an employee first joins a company and then gets up to speed or ramps up). The node graph generation system 200 can identify metrics associated with a successful ramp-up period based on identifying member nodes that are associated with desired performance outcomes based on reaching desired stages in an opportunity record object (i.e. by analyzing how successful employees had ramped in the past). Thus, by analyzing electronic activities and a corresponding system of record to determine data driven metrics associated with desired performance outcomes determined by linking activities with record objects describing process stages (e.g., an opportunity record) in the system of record, the node graph generation system 200 can generate or identify goals to set for member nodes that are in a ramp-up period or other time interval, such as during a performance improvement plan (a plan, set up by employee's manager to bring the employee to optimal performance after a period of poor performance). The node graph generation system 200 can further reevaluate the member node's metrics to update the goals or set new goals by comparing current metrics (e.g., actual actions or performance) associated with the member node's current electronic activities with the desired metrics (e.g., planned actions or performance) for electronic activities correlated with the desired performance outcome or result.

In some embodiments, the system 200 can be configured to compare performances of employees of a company by monitoring the employee's contribution to opportunity record objects and the progression of the stages the opportunity record object goes through. For instance, a high performing employee may be involved in electronic activities that are linked to opportunity record objects that advance from one of the stages to another stage much quicker than another employee with the same role. Similarly, a high performing employee may be involved in electronic activities that are linked to a greater number of opportunity record objects that advance from one of the stages to another stage than another employee with the same role. as such, by tracking the opportunity record objects with which an employee is linked, a performance of the employee can be determined and the employee's metrics can be used to set certain benchmarks that can then be used to determine a performance of another employee with a similar role or generate a ramp up schedule based on the employee's metrics. For example, the node graph generation system 200 can determine that when a member node completes 25 calls in a week, reaches out to 10 companies in a week, has 5 in-person meetings in a week, and then writes 100 emails in the same week, then the member node should be able to complete a number of deals or advance a desired number of stages in one or more deals or otherwise achieve an expected performance outcome after a certain time (e.g., a time delay between input activities and outcome results). The metric can refer to or include an attribute of an activity, such as an amount of the activity. The metric can be a binary value that indicates a yes or no, such as “did you have a meeting with 10 people”, with a value of 1 or 0 indicated yes or no, respectively. In some cases, the metric can be a count, a ratio, a time value, or a percentage value, based on any combination/formula, calculated from any number of data points in the member node graph or system of records. The metrics can vary in granularity based on the data the node graph generation system 200 can analyze via electronic activities or one or more systems of record. Based on previous or historical activity, the node graph generation system 200 can predict, forecast or estimate what activity should occur to achieve a desired outcome, and propose or set goals for a member node or group node accordingly. The node graph generation system 200 (e.g., via the electronic activity linking engine) can correlate the electronic activities with the stages or desired outcomes as stored or determined in the system of record or an opportunity record object thereof. The electronic activity linking engine can match, correlate, link or otherwise associate electronic activities with outcomes (e.g., advancing stages, won, lost, etc.) stored in the system of record.

The node graph generation system 200 can generate an automated employee ramp-up schedule based on the previously identified high performing member nodes based on internal user data. The node graph generation system 200 identifies high performing member nodes based on electronic activities associated with the member nodes matching desired outcomes as indicated in opportunity record objects stored in a system of record (e.g., system of record 9360) or stored in a shadow or temporary system of record associated with the node graph generation system 200, or otherwise stored in a master system of record. With this automatically generated ramp-up schedule containing metrics for electronic activities that is correlated with high performing member nodes, the node graph generation system 200 can provide goals or recommendations to new member nodes that are beginning a new job or new role at a company. Such recommendations can be especially relevant for employees in sales, customer success, recruiting, or other functions.

To generate the ramp-up schedule for a new member node (e.g., a new hire), the node graph generation system 200 can identify a high performing member node that has a node profile that is similar to the member node profile of the new member node. The node graph generation system 200 can compare member node profiles based on values of fields of the member node profiles, such as geographic area, type of industry, experience, or any other field of the member node profile. The node graph generation system 200 can then identify metrics associated with the similar member node profile of the high performing member node and generate a ramp-up schedule using the metrics.

To identify the metrics, the node graph generation system 200 can normalize the metrics for a time interval. The node graph generation system 200 can identify metrics for the high performing member node that occurred during a time interval that is similar or relevant to the new member node profile. For example, the node graph generation system 200 can identify the first two weeks of employment by determining when the first email was actually sent by the employee, and then identifying the metrics for electronic activities that correspond to the first two weeks of the high performing member node's employing at the company. These first two weeks may not indicate a high performance. For example, the high performing member node may not have been high performing with reference to desired outcomes in matching opportunity record objects in a system of record for another 6 months; however, the metrics associated with electronic activities that occurred in the first two weeks or other time interval prior to the desired performance outcomes may nonetheless be indicative or relevant to the high performance level of the high performing member node. Thus, the node graph generation system 200 can select the metrics of electronic activities that occurred in the first two weeks and provide those metrics as goals or target goals or target metrics for the new member node without setting a goal or expectation that the member node achieve a desired opportunity stage in the initial time interval, but, instead, with the goal that the new member node may achieve the desired performance with references to opportunity stages during a later or subsequent time interval. The node graph generation system 200 can correlate metrics to outcomes (e.g., all metrics of electronic activities that correlate with positive outcome), and then compare new employee to a previously successful employee.

The node graph generation system 200 can normalize the time interval or otherwise account for environmental factors or external factors associated with the time interval that can affect the metrics associated with electronic activities or performance outcomes. For example, the node graph generation system can take into account a seasonal component by detecting a reduction in electronic activities during a vacation time interval. The node graph generation system 200 can determine or detect the vacation based on identifying an automatic out of office reply in outbound electronic activities corresponding to the member node. The node graph generation system 200 can determine or detect the vacation based on identifying a vacation calendar entry electronic activity corresponding to the member node. The node graph generation system 200 can identify the vacation responsive to determining that a volume of electronic activity or responsiveness to electronic activities during a predetermined time interval is below a threshold for the email account of the node profile, or the hours during which emails are sent vary from a traditional time range or time zone for the member node (e.g., whether electronic activities or communications are clustered around business hours). By determining that the new member node may be on vacation—or that a high performing member node's metrics were associated with a vacation—the node graph generation system 200 can remove or filter out metrics or data during the vacation period so as not to set improper or erroneous goals that might be faulty due to a vacation time interval, or so as not to determine that the new member node is underperforming or not meeting goals due to the new member node being on vacation.

The node graph generation system 200 (e.g., via recommendation engine 275) can provide the target goal or recommendation to the member node, or a manager member node that may then propagate the target goal to employee member nodes. A manager member node can refer or correspond to a person whose role is a manager of employees or a team of people. The member node profile can include a field that denominates a role of the member, such as manager or employee. The member node profile can further include a field that denominates who the manager is, such as a “managed by” field.

In some embodiments, the recommendation engine 275 can include or interface with a machine learning engine that obtains feedback from a manager member node and adjusts the recommendations or target goals accordingly. For example, the node graph generation system 200 can identify manager member nodes that are linked to employee member nodes that are performing with a desired outcome based on a system of record. The node graph generation system 200 can further identify that when new employee member nodes are linked or join the network of the manager member node, the new employee member node ramps up in a desired time interval and to a desired performance level. The node graph generation system 200 can receive human input from a manager corresponding to a manager member node. Based on the human input, the node graph generation system 200 can determine that the manager member node sets goals that are effective or successful in improving the performance of the employee member nodes. The node graph generation system 200 can receive, via the manager member node or one or more employee member nodes, the target goals and input these target goals into a machine learning engine or otherwise compare the input target goals with automatically generated target goals to tune or update the generation of target goals. Thus, the node graph generation system 200 (or recommendation engine 275) can receive human input from high performing managers in order to update the recommendation engine 275 and improve the generation of recommendations or goals for member nodes.

The node graph generation system 200 can include a performance module 280 designed and configured to determine a performance of a member node. The performance module 280 can identify when metrics of a member node do not meet or exceed the target goal metrics set for the member node.

The node graph generation system 200 can recommend to the manager to establish, responsive to detecting that the metrics for electronic activities for a member node do not satisfy the target goals, a performance improvement plan for the member node. The performance improvement plan can be based on a difference between the member nodes' current metrics and the target metrics. The performance improvement plan can be further based on identifying a similar member node to the underperforming member node that also previously underwent a performance improvement plan but is high performing now. The performance improvement plan can be based on human input received from a manager member node. Thus, the node graph generation system 200 (e.g., via recommendation engine 275) can generate a customized or tailored performance improvement plan that is based on a similar member node whose activity levels and goal attainment indicates that the similar member node successfully completed a performance improvement plan and is now a high performing member node.

The node graph generation system 200 can generate this customized or tailored performance improvement plan using human input from a manager that is deemed, by the recommendation engine 275, to be a high performing manager.

The node graph generation system 200 can set performance benchmarks for a member node, a plurality of member nodes (for example, a team of member nodes), group nodes, industry nodes representing a plurality of group nodes belonging to the same industry, nodes within a geographic territory, or any other collection or group of nodes. The node graph generation system 200 can establish benchmarks for performance based on analyzing the performance of one or more groups of nodes having similar characteristics. The node graph generation system 200 can identify similar groups of nodes based on a group size (e.g., number of member nodes in the group node), revenue of the group node, industry associated with the group node, geographic region of the group node, or other characteristic. These characteristics can be set or stored or inferred from a group node profile associated with the group node of a group node graph.

The node graph generation system 200 can generate income estimates for member nodes based on performance outcomes derived from electronic activities associated with the member node. For example, the node graph generation system 200 can determine how performance outcomes map to income, for example in sales, and then estimate income based on metrics of electronic activities that match the performance outcome stored in an opportunity record object in a system of record. The node graph generation system 200 can perform a deal-by-deal benchmarking to determine an income estimate. The system 200 can identify successful historical deals that are similar to a target deal. The system 200 can determine whether the types or quantities of electronic activities (or other metrics associated with electronic activities) associated with the successful historical deals are similar to the electronic activities metrics that are occurring in the target deal. If the system 200 determines that the target deal is on track to be a successful deal based on the electronic activities metrics for historical similar deals that were successful, then the system 200 can determine that the target deal will be successful, or more likely to close, so the representative member node for the deal is likely to keep a commission. The node graph generation system 200 can provide an indication to the member node on a periodic or other time interval with current metrics of electronic activities and target metrics for electronic activities in order to achieve the desired income. Based on deal-by-deal benchmarking, the system 200 can determine how many deals of what type the member node needs to close in a year to make the desired income. Based on the number and type of deals, the system 200 can set the goal electronic activities metrics for the member node that are likely to result in closing the deals. For example, a member node may want to make $50,000 per year, then the node graph generation system 200 can notify the member node that they need to have 10 in-person external meetings per week, write 100 emails to external contacts, and make 25 phone calls to external contacts (e.g., metrics for electronic activities that are were associated with similar deals that were successful).

The node graph generation system 200 can detect, based on analyzing electronic activities, whether the member node is satisfying the target goals. If the member node is not satisfying the target goals to achieve the desired income, the node graph generation system 200 can predict the reduction in income relative to the desired income and notify the member node of the reduction in income that may result from missing the target goals. The system 200 can tie current performance level to future projected wins (e.g., successful deals), and hence to future projected income.

In some embodiments, an employee's compensation may be based on the performance of the team that the employee is managing. For such an employee, such as a team manager, the node graph generation system 200 can help establish a compensation structure for the team manager member node that is based on the performance of his team, which is based on the individual performance outcomes of the employee member nodes the team manager manages. In some embodiments, the system 200 can analyze electronic activities (and corresponding record objects to which the electronic activities are matched) relating to the team managed by the team manager to determine or predict the performance of the team. The system 200 can then generate specific actions that the team manager or his team can or should take to improve the performance of the team or to achieve previously established goals. More generally, the node graph generation system 200 can establish goal outcomes and recommend actions based on analyzing electronic activities or accessing or analyzing systems of record. The node graph generation system 200 (e.g., via performance module 280) can compare electronic activity metrics or aggregated activity metadata for similar processes (e.g., sales deals, recruitment process, etc.) to determine a performance outcome for the member node participating in the process. The node graph generation system 200 can generate such goal outcomes or recommend actions (e.g., electronic activities) with varying granularity, for instance, hourly, daily, weekly, bi-weekly or monthly, among others. The node graph generation system 200 can establish a sales compensation system based on analyzing electronic activities or accessing or analyzing systems of record. Thus, the node graph generation system 200 can automate the process of goal setting for team management, or setting team management on autopilot, based analyzing electronic activities or accessing or analyzing systems of record.

The node graph generation system 200 can set a manager member node goal of having every employee member node perform a certain number and type of electronic activities in a certain time interval. In some cases, the manager member node goal can include aggregate activity metadata associated with electronic activities, such as response rates from C-level executives, meeting attendance rates, or meeting reschedule rates. The node graph generation system 200 can detect that the goal was not met by a first employee member node, and then perform an early warning prediction that the first employee member node may not be ramping up on time. The node graph generation system 200 can tie this missed goal detection with an indication that the first employee member node may not be ramping up on time. For example, out of 50 member nodes that succeeded at a company, their metrics trended in accordance with curve X, whereas the first employee member node's metrics trend in accordance with curve Y, which may not intersect with curve X, therefore the first employee member node may not be ramping up in a satisfactory manner. Metrics can indicate a cadence, response time to emails, number of calls, etc.

The node graph generation system 200 can identify different patterns for different industries or different types of processes (e.g., sales, recruiting, etc.). The node graph generation system 200 can establish goals for each type of deal or opportunity or industry based on the patterns. The node graph generation system 200 can, for example, establish patterns to advance stage with a specific OCR or champion. For example, the node graph generation system 200 can establish metrics for electronic activities that are tailored or customized for the specific OCR with which the seller is interacting. For example, the node graph generation system 200 can estimate for a specific deal to advance to a next stage, there should be a certain number of electronic activities with the OCR during a time interval; so, the node graph generation system 200 can set that as the goal for the time interval. The number of electronic activities can be based on or include a number of people in a meeting, average seniority of people in a meeting, or other granular indicators.

The node graph generation system 200 can generate an effort estimation model for each member node based on electronic activities or metrics thereof. The metrics can indicate low responsiveness, empty times on calendar during key business hours, or other predictors that someone is not putting in a threshold level effort. The node graph generation system 200 can detect a drop off in metrics as a drop off in effort. The node graph generation system 200 can detect a drop off or lack of participation in certain types of activities as an indication of low effort and thus predict a person being disengaged and preparing to leave the company.

### 13. Systems and Methods for Assigning Employees to Business Processes Including Leads, Accounts, and Opportunities

Companies typically assign employees to certain leads or accounts in a round robin fashion. As new leads or accounts are identified, a company may assign a different sales rep to the lead or account without attempting to match the sales rep to the lead or account. However, none of the assignments of sales reps to leads or accounts or opportunities with such accounts is data driven, automated or objective in nature.

As described herein, companies can maintain various systems of record, including a customer relationship management system, which the company can use as a holding system for descriptions of business processes. The system of record can include lead record objects identifying leads that the company may pursue, account record objects identifying accounts to which the company sells one or more products or services, opportunity record objects identifying deals or opportunities between the company and the account, among others.

The present disclosure describes systems and methods for automatically assigning employees of a company to certain business processes of the company using a data driven approach. Before describing specific examples of different business processes to which employees of a company can be assigned, it should be appreciated that the system 200 can automatically assign employees to certain business processes by taking advantage or utilizing other aspects of the system 200.

As described herein, the system 200 can be configured to receive and parse electronic activities, link such electronic activities to node profiles of a node graph, update the node profiles based on the contents of the electronic activities, match the electronic activities to record objects of one or more systems of record of companies, generate activity patterns of node profiles including but not limited to communication styles, response rates, response times, communication mode preferences, among others. These insights and others can be determined by the system 200 based on the electronic activities the system 200 parses.

The system 200 can be configured to automatically assign at least one employee of a company to one or more record objects or provide recommendations to the company (for instance, the data source provider) to assign the at least one employee to the one or more record objects. The system 200 can be configured to automatically assign or generate a recommendation to assign a business process or associated record object to an employee of a company associated with the business process. Perhaps, more generally, the system 200 can automatically match or generate a recommendation to match or pair an employee of a company and a record object of a system of record of the company.

In some embodiments, to do so, the system 200 can be configured to maintain, for each employee of the company, an availability of the employee based on a status of one or more record objects to which the employee is assigned. In some embodiments, the employee can be assigned to a first number of lead record objects, a second number of account record objects and a third number of opportunity record objects. The system 200 can further determine, for each of the opportunity record objects, a stage of the opportunity record object. Moreover, the system can determine an amount of time the employee needs to spend on the opportunity based on the stage of the opportunity record object, a size of the deal associated with the opportunity, an expected or predicted time frame for closing the opportunity, and other parameters associated with the opportunity record object. The system can determine, based on each record object to which the employee is assigned, an availability schedule of the employee that identifies the employee's availability during various time periods, including for example, the next week, the next two weeks, the next month, the next quarter, the next year, among others.

The system 200 can be configured to automatically match or generate a recommendation to match or pair an employee of a company and a record object of a system of record of the company by using one or more rules that may be specific for different types of record objects. In some embodiments, the rules can be learned by analyzing previous matches between employees and record objects and the success or failures of such matches. The rules can be learned using machine learning or other techniques.

The following sections describe how the system can automatically match or generate a recommendation to match or pair an employee of a company and different types of record objects of a system of record of the company.

### A. Matching Employees and Lead Record Objects

This section relates to matching employees and lead record objects, assigning employees to lead record objects or assigning lead record objects to employees. A lead record object can identify a person who can be an early interest for the company. Determining how successful an assignment of a lead to an employee is likely to be is based on several factors. Some of these factors include i) a quality of the lead; ii) behaviors or business practices of the employee; iii) behaviors or business practices of the lead; and iv) availability of the employee to service the lead, among others. Out of these factors, the availability of the employee to service the lead can be a more important factor. This makes sense because a salesperson currently working on 5 late stage deals likely will not have the availability to service the lead, which will result in the company losing the lead because the salesperson was unable to commit enough time to building a relationship with the lead. Examples of behaviors or business practices of the employee can be their preferences to want phone calls over emails or in person meetings, a desired time of day factoring in their time zone during which the employee likes to communicate with leads, or an employee's comfort level with dealing with leads having certain titles, for example, CIO, CEO or other executive level leads. Similarly, the behaviors, preferences and business practices of the lead can also be relevant.

In one embodiment, the system 200 can be configured to first determine, for a given lead, a plurality of employees of the company that may be potentially be assigned to the lead. These employees may be salespersons. The system 200 can then determine the availability of each of the salespersons and based on their respective availabilities, the system 200 can select a subset of the salespersons as candidate salespersons. The system can then determine, for each candidate salesperson, the behaviors, preferences and business practices of the candidate salesperson that the system 200 can derive from parsing electronic activities linked to a node profile of the candidate salesperson. The system 200 can then compare the determined behaviors, preferences and business practices of the candidate salesperson to behaviors, preferences and business practices of the lead (which can also be determined by the system 200 by parsing electronic activities linked to a node profile of the lead). In some embodiments, the system 200 can be configured to determine the behaviors, preferences and business practices of the candidate salesperson as it relates to the lead by only analyzing electronic activities exchanged between the salesperson and other leads in the past. Similarly, the system 200 can be configured to determine the behaviors, preferences and business practices of the lead as it relates to the plurality of candidate salespersons by only analyzing electronic activities exchanged between the lead and other salespersons in the past. The system 200 can then determine a match score between the candidate salesperson and the lead based on the comparison and either automatically assign the candidate salesperson to the lead or vice versa or provide a recommendation to assign the candidate salesperson to the lead or vice versa to the administrator or user of the system of record in charge for assigning leads to employees.

In some embodiments, the system 200 can use other signals or factors for matching leads to employees. For instance, if the system 200 can determine if the lead has any prior connection with any of the candidate salespersons and also determine a connection strength and type of connection between the lead and the candidate salesperson. As described herein, the system 200 can maintain a connection strength between node profiles of the system 200 and as such, the system can use the connection strength between the lead and the candidate salespersons as a factor to determine which candidate salesperson to match to the lead.

The system 200 can be configured to assign different weights to different factors used for matching leads and employees. In some embodiments, the system can enable each company to establish its own rules or policies for recommending matches between leads and employees. In some embodiments, the system 200 can be configured to train a machine learning model to match leads and salespersons based on analyzing a salesperson's matches with leads in the past as well as analyzing the lead's matches with other salespersons in the past.

By way of this solution, the system 200 can reduce the number of candidate salespersons the company needs to consider for each new lead thereby allowing the person responsible for assigning leads to employees to spend less time pairing leads to employees while improving the likelihood of success of converting the lead by selecting candidate employees that are most likely going to succeed with this lead based on objectively analyzing historical electronic activities. Moreover, at present, companies are focusing on lead generation without optimizing the conversion of existing leads. The solution described herein aims to determine which employee is most likely to convert the lead to optimize the company's ability to convert each and every lead of the company.

### B. Matching Employees and Account Record Objects

In contrast to the concept of matching employees and leads described above, this section relates to matching employees to accounts. An account or an account record object corresponds to a customer of the company. Each account can be linked to one or more lead record objects and opportunity record objects. In contrast to lead assignments described in the previous section, account assignment is similar except that a lead is one person while an account includes a group of people.

The system 200 can be configured to identify an account of a company to which to assign one or more employees of the company to service the account. The system 200 can be configured to identify each of the contacts at the account. The contacts may be identified by analyzing the contact record objects of the system of record to identify which contacts are linked to the account. In some embodiments, the system can utilize the node graph of the system to analyze ode profiles that currently work at the account. The system can then run an analysis for each employee of the plurality of employees of the company that may be a candidate to service the account based on the employee's function or job description. Upon selecting a set of candidate employees from the plurality of employees of the company, the system 200 can determine, for each employee, a connection strength between the employee and each of the contacts at the account. The system can then aggregate, for the employee, the connection strengths between the employee and each of the contacts by applying different weights based on the role, title or function of the contact within the account, which can all be determined by the system through the system of record or the node profiles maintained by the system 200. The system can then determine, from the aggregated connection strengths of each of the plurality of employees, at least one employee to assign or recommend assigning to the account. As mentioned above, the contacts at the account with which the employee has relationships can be weighted based on their role, title or function.

In some embodiments, the system 200 can take into account other factors other than connection strengths prior to assigning the employee to the account. In particular, the system 200 can also consider the geographical proximity between the employee and the account or the contacts within the account. The system 200 can also consider the employee's selling style or other behavioral patterns and compare them to the buying style of the contacts within the account to determine whether or not to assign the employee to the account. In addition, the system 200 can take into account past experiences of the employee with the contacts or the account itself. For instance, the system 200 can determine if the employee has previously worked for or with the account at a previous job. The system 200 can also determine if the employee has previously worked with any of the contacts included in the account. The system 200 can also determine if the employee has previously worked with similar types of accounts, for instance, if the account is Verizon, the system can determine if the employee has worked with AT&T given that AT&T is in the same sector as Verizon and so the employee may be a better fit for an account such as Verizon. In some embodiments, the system 200 an also determine an availability of the employee to determine if the employee has the capacity to service the account.

In some embodiments, the system can determine a target persona for the account. For instance, if the account is a marketing department of a customer, the system can be configured to generate a target persona that corresponds to the marketing department as opposed to an accounts department. The system can then attempt to identify employees within the company that most closely match the target persona corresponding to the marketing department as this employee will be most likely to best serve the account.

The system 200 can also be configured to take into account other employees to assign to the account as part of a sales team. As such, the system 200 may be configured to determine whether or not to assign an employee to the account based on which other employees are already assigned to the account or are candidates to be assigned to the account. The system 200 can be configured to assign an employee to the account based on the employee's relationship with other employees who are already assigned to the account, for instance, the employee is part of 3 other sales teams that include the other employees.

In addition, the system 200 can be configured to recommend additional employees to assign to the account based on selecting an employee to assign to the account. For instance, the system 200 can identify a first employee as a sales representative to the account. The sales representative generally works with a sales engineer and an account executive when selling to a company. As such, the system 200 can be configured to select a sales engineer from a plurality of candidate sales engineers and select an account executive from a plurality of candidate account executives to assign to the account based on determining that the sales engineer and the account executive have been included in sales teams with the sales representative for other accounts.

In some embodiments, the system 200 can be configured to recommend overlay resources like sales engineers to the account. The sales engineer that is recommended may be selected for recommendation responsive to the system determining that the sales engineer also has connections to the account. In addition, the system 200 can further recommend executives on the company side to which to recommend or assign to the account. By generating these additional recommendations of employees to the account, the system can be configured to automatically recommend or generate account team recommendations that the company can use to build account teams. As described herein, these account teams can be based on their relationships with contacts at the account, their past experiences with the account their past experiences working with each other on other accounts, as well as their availability to service the account, among others.

In some embodiments, the system can identify one or more people at the account who may be considered to form the buying group. The buying group can be determined by the system 200 using the node graph of the system 200 or from other systems of record accessible to the system 200. The system 200 can be configured to identify employees to assign to the account based on the target persona of the account as well as the buying group of the account. In some such embodiments, the system can adopt the same techniques and methodology described herein but adjust weights of certain factors based on the target persona of the account as well as the buying group of the account.

As described herein, the system 200 can be configured to detect account teams from electronic activities that are matched to record objects corresponding to account record objects or opportunity record objects. Detecting that an employee belongs to an account team based on electronic activities can be useful to the system 200 for matching the electronic activities identifying the employee to the appropriate record object of a system of record, among others. In some embodiments, an account team can be determined from the system of record based on linking contact record objects to an account record object, for instance. However, it should be appreciated that the system 200, as described with respect to Section 12, is configured to provide recommendations of employees to add to existing account teams or create new account teams for new accounts.

As described above with respect to matching electronic activities to record objects, the system 200 can be configured to identify candidate record objects to match electronic activities based on account teams. By being able to expand the account teams or verify if an employee should be added to an account team, the system 200 can be configured to improve its ability to match electronic activities to record objects by better identifying record objects using matching strategies involving account teams.

### C. Matching Employees and Opportunity Record Objects

The system can be configured to also be configured to automatically assign or recommend assigning an employee to an opportunity record object. The system can identify employees to match to opportunity record objects in a manner similar to lead record objects and account record objects. As opportunities are business processes that need active involvement in the short term, selecting an employee to assign to the opportunity, the system can give more importance to the employee's availability in the short term relative to when the system selects an employee to match to an account. The system can be configured to determine, for each employee, their current load or available capacity based on the number of opportunities the employee is working on, what stage each of the opportunities is in, among others. As such, a described here, the system's ability to predict stage classification of opportunity record objects can be used to determine the employee's availability and based on the employee's availability, a recommendation to assign the employee to one or more opportunities that the employee is not currently assigned to.

The system can be configured to identify, for a given opportunity, one or more opportunity contact roles associated with the opportunity as well as other contacts at the account level that are involved with the activity. The system can then determine to identify candidate employees that would be a good fit for the opportunity based on a comparison of the candidate employee and the contacts involved or likely to be involved with the opportunity. Based on the determination, the system can provide a recommendation to add a candidate employee to the account team servicing the opportunity record object. As described above with respect to matching employees and lead record objects and account record objects, the system can determine similar factors to determine how good a fit the candidate employee will be for the opportunity.

### D. Matching Employees and Named Account Lists

The system 200 can be configured to match employees to one or more account lists. In a scenario where a new employee joins a company, a supervisor may be assigned to assign the employee to multiple accounts or leads, among others. At present, the supervisor may simply assign the employees to accounts based on a geographical location of the employee and corresponding locations of the accounts. However, assigning employees to accounts simply based on location matching fails to optimize the employee's ability to generate new leads and opportunities.

In some embodiments, the system 200 can be configured to generate a list of accounts to which to assign an employee of the company. In some embodiments, the system 200 can receive a request from a user of the system 200 to assign or identify accounts of the company to the employee. The system 200 can first identify all of the accounts of the company to which the system 200 can possibly match the employee. The system 200 can then determine, for each account, one or more contacts at the account with which the employee has a connection. The system 200 can use the node profiles and node graph to determine these contacts. The system 200 can then determine, for each contact with which the employee has a connection, a connection strength between the contact and the employee. The system 200 can then weight each of these connections based on the account and the role of the contact within the contact. The system 200 can then determine an aggregated score between the account and the employee based on the weights and connection strengths of the employee with the contacts of the account. The system can compute the aggregated score also by factoring in a location of the employee relative to the account, a time zone of the employee relative to the account, a selling style or communication style of the employee relative to the buying style or communication system of the contacts within the account. The system can then generate a list of accounts to which to match the employee based on the aggregated scores between the employee and the respective account. It should be appreciated that other factors, such as the employee's availability, timing of potential opportunities of the account, other employees that may likely form the account team, can also be factors in computing the aggregated score between the employee and the respective account.

### E. Matching Employees and Territories

Some companies may maintain one or more systems of record in which employees are assigned to territories, such as geographical regions. In some such cases, the system 200 can be configured to assign employees to territories, which may be assigned to certain accounts. Similar to how the system 200 can determine an aggregate score for each account described above, the system 200 can be configured to assign employees to territories based on determining an aggregate score between the territory and the employee by determining individual scores between accounts within the territory and the employee.

It should be appreciated that matching employees to various record objects or business processes described above can be based on objective data that is parsed from electronic activities involving the employee or electronic activities involving leads or contacts at the accounts. As such, the system 200 can be configured to rely on certain electronic activities when determining which record objects or business processes to match or assign or which employee to assign or match to the record objects or business processes of the company. In this way, a data-driven approach to selecting employees to assign to accounts can be achieved, which can result in better outcomes for the employee, the account, and the company.

It should further be appreciated that similar methodology can be used by the system 200 for identifying potential candidates to suggest to a company to hire as employees. The system 200 can analyze a candidate's connections and communication style from electronic activities linked to the node profile of the candidate and use that information to compare to accounts of the company to determine if the employee will be a good fit. Similarly, for a person looking to join new company the system 200 can identify potential candidate companies to the person based on the person's connections and communication style determined by the system 200 from electronic activities linked to the node profile of the person and information about the candidate companies and their respective accounts also maintained by the system 200.

### 14. Systems and Methods for Generating Data Recommendations Based on an Immutable Member Node Network

At least one aspect of the present disclosure is directed to systems and methods for generating data recommendations based on an immutable member node network. The immutable member node network can refer to or include a member node network containing member nodes connected to one or more other member nodes. The member nodes can contain a member node profile that is generated by the node graph generation system 200 (or node profile manager 220) using electronic activity information or information from a master system of record. By using electronic activity information or a master system of record generated and maintained by the node graph generation system 200, the node graph generation system 200 can generate data recommendation using the immutable member node network.

The member node network may be immutable in that the member node network may be accurate and not contain erroneous data, or lack data with a confidence score that falls below a threshold. The node graph generation system 200, using the member node network (e.g., member node graph) can match member nodes to a potential group node, job, account, or opportunity based on the member node profile matching profiles, metrics or parameters associated with the group node, job, account or opportunity.

For example, the node graph generation system 200 can determine that a particular member node is represented by a member node profile that includes fields and values for the fields. The node graph generation system 200 can further determine, via a member node performance module 280, a performance score as well as performance metrics for the member node. The performance information can be correlated with metrics associated with electronic activities. The performance metrics can be granular and correspond to profile values. For example, the member node profile performance information can indicate that a member node has a high performance level when the member node performs electronic activities that include at least a first number of in-person meetings with C-level executives. For example, if the member node has five in-person meetings with C-level executives in a week, then the performance module 280 can determine that the member node is performing well based on historical performance information for the member node or similar deals. The node graph generation system 200 (or member node performance module 280) can determine the high performance level (e.g., relative to an average performance level across member nodes or a subset of member nodes). The node graph generation system 200 can then identify group nodes or group profiles associated with group nodes or companies that match the profile values correlated to the high performance level of the member node.

As described above also with respect to Section 12, the system 200 can be configured to utilize information included in the node graph to match candidate employee and companies based on the candidate employee's connections with one or more accounts of the companies to which the system 200 determines a match. In some embodiments, the system 200 can determine a match based on a candidate employee's selling style and a buying style of a buyer's group of an opportunity linked to one of the accounts of a company. It should be appreciated that the system 200 can look at other signals too when making such matches and not rely simply on matching according to a selling style or for a single opportunity. In some embodiments, the system 200 can identify an employee within a company that should be put on an account team of an opportunity record object based on the selling style of the employee (or other factors such as availability, connections to the buyer group, among others).

Thus, the node graph generation system 200 can match member nodes to a group node based on performance characteristics or other metrics of the member node and the group node that are derived, inferred, or otherwise determined using electronic activities from data source providers 9350 and record objects from one or more systems of record. The node graph generation system 200 can use the electronic activities and the data in the systems of record to generation a performance profile for a member node, which can be stored in a master member node network or immutable member node network. The member node network can be immutable because it is not self-written or self-reported by individuals; instead, the node graph generation system 200 generates the performance profile and member node profile using electronic activities and systems of record, which is an independent, factual, and objective source of activity information. The node graph generation system 200 can generate the group node network containing group profiles for group nodes. The node graph generations system 200 can identify granular values that are correlated with desired performance or outcomes based on stages of opportunities or stages of other business processes, stored in record objects of a system of record or one or more systems of record.

As described herein and supplemental to the description of various terms provided above, electronic activities can include emails, electronic calendar events, electronic meetings, phone call logs, instant messages, other any other electronic communications generated by a node, received by a node, exchanged between nodes or otherwise stored on an electronic server configured to provide electronic activities to the data processing system 9300.

An individual or member node can be an electronic representation of a user, person, account of a person or user, an employee, a bot, or any other entity that may have an account or an identifier that the data processing system can generate a node profile for. A group node can be an electronic representation of an enterprise, a company, an organization, an employer, a team of employees or people, or a plurality of member nodes that can be treated as a single entity. A node profile can be an electronic representation of a profile of a member node or a group node. The node profile can include fields. Each field can include one or more values. An example field can be an email address. An example value can be john.smith@example.com. A value of a field can include an array of data points identifying occurrences of the value. Each value can have a confidence score. A data point can identify an electronic activity or other piece of information that contributes the value to the field. The data point can include or identify a source of the electronic activity, a trust score of the source of the data point, a time or recency of the electronic activity and a contribution score. The source of the electronic activity can be a mail server, a system of record, or any other repository of electronic activities.

A trust score of the source of the data point can indicate a trustworthiness of the source of the data point. The trust score of the source can be based on a completeness of system of record maintained by the source. The trust score can also serve as an indication of how reliable the source may be.

A contribution score of the data point can indicate how much the data point contributes towards a confidence score of the value associated with the data point. The contribution score can be based on the trust score of the source, a health score of the source, and a time at which the data point was generated or last updated.

A confidence score of the value can indicate a level of certainty that the value of the field is a current value of the field. The higher the confidence score, the more certain the value of the field is the current value. The confidence score can be based on the contribution scores of individual data points associated with the value. The confidence score of the value can also depend on the corresponding confidence scores of other values of the field, or the contribution scores of data points associated with other values of the field.

A confidence score generally relates to a level of confidence that a certain piece of information is accurate. As used herein, a confidence score of a piece of information, such as an assigned tag, a value of a field of a node profile, a stage classification prediction, a record object match, can indicate a level of confidence that the piece of information is accurate. The confidence score of the piece of information can change based on a temporal basis. A node profile can include a first email address corresponding to a first job and a second email corresponding to a subsequent job. Each of the two email addresses are at respective points in time, accurate and valid. As the person switches jobs, the first email address is no longer valid but the confidence score associated with the email address can in some embodiments, remain high indicating that the first email address belongs to the node profile. Similarly, the second email address also belongs to the node profile and therefore also has a high confidence score. After the system determines that the second email address is active and functioning, the system can assign a higher confidence score to the second email address relative to the first email address since the contribution scores provided by recent data points (for example, recent electronic activities identifying the second email address) can contribute towards the higher confidence score. Similarly, any tags that are assigned to electronic activities identifying bounce back activity related to the first email address (indicating that the first email address is no longer active) can reduce the confidence score of the first electronic activity.

The health score of the source can indicate a level of health of the source. The health of the source can include a completeness of the source (for example, a system of record), an accuracy of the data included in the source, a frequency at which the data in the source is updated, among others.

A connection strength between two nodes can be based on the electronic activities associated with both the nodes. In some embodiments, each electronic activity can be used by the system to determine a connection strength between the two nodes. The contribution of each electronic activity towards the connection strength can diminish over time as older electronic activities may indicate a past connection but do not indicate a current status of the connection strength between the two nodes.

The time decaying relevancy score of an electronic activity can indicate how relevant the electronic activity is for determining a connection strength between two nodes exchanged between or otherwise associated with the two nodes. The connection strength between two nodes can be based on the time decaying relevancy scores of all of the electronic activities exchanged between or otherwise associated with the two nodes.

As further described herein, electronic activities can be linked to or matched to record objects. Record objects can be maintained in a shadow system of record maintained by the system 9300 or in some embodiments, linked or matched to record objects maintained in master system of records that are maintained by customers or enterprises.

### 15. Matching Electronic Activities Directly to Record Objects of Systems of Record

As described above, the system described herein can match electronic activities with one or more record objects. The system can match the electronic activities in a single-tenant or multi-tenant configuration of the system. For example, in a single-tenant configuration, the system can receive or access electronic activities from a single data source provider and match the electronic activities to record objects of a system of record of the data source provider from which the electronic activities were received or accessed. In a multi-tenant configuration, the system can receive or access electronic activities from multiple data source providers and match the electronic activities to record objects of a system of record of the respective data source provider from which the electronic activities were received or accessed. As described herein, the system can automatically match, link, or otherwise associate the electronic activities with one or more record objects. For an electronic activity that is eligible or qualifies to be matched with one or more record objects, the system can identify one or more set of rules or rule sets. Using the rule sets, the system can identify candidate record objects. The system can then rank the identified candidate record objects to select one or more record objects with which to associate the electronic activity. The system can then store an association between the electronic activity and the selected one or more record objects.

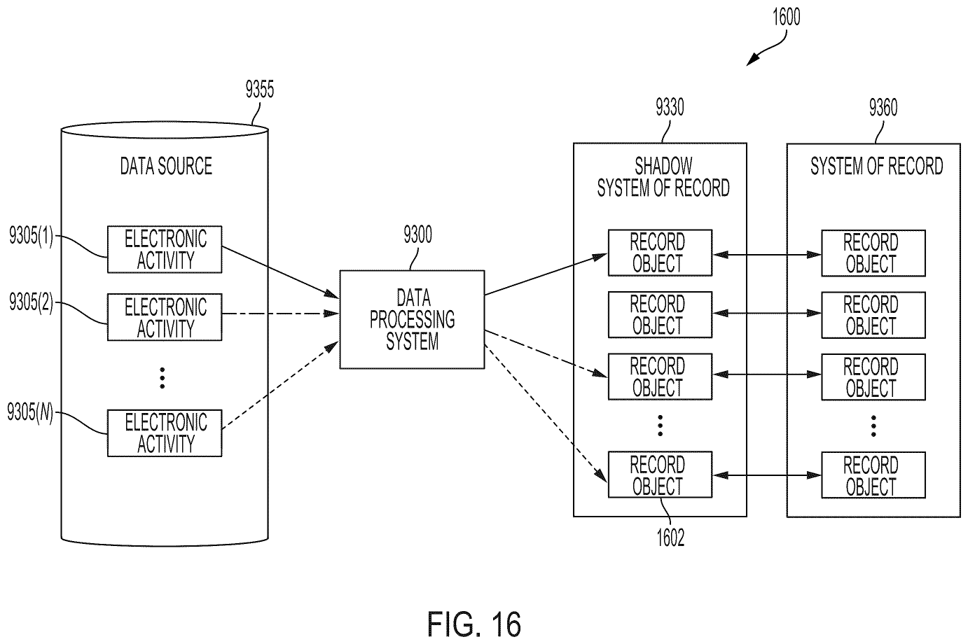

FIG. 16 illustrates a block diagram of an example process flow 1600 for processing electronic activities in a single-tenant configuration. Also, with reference to FIGS. 3 and 4, among others, the data processing system 9300 can be in communication with one or more data source providers 9350. Each of the data source providers 9350 can include a data source 9355. FIG. 16 illustrates an example of a single-tenant system where the electronic activities 9305 from a single tenant (e.g., the data source provider 9350 that includes the data source 9355) is matched to the record objects 1602 of a single shadow system of record 9330. The single shadow system of record 9330 can be associated with the data source provider 9350 that provided the electronic activity. For example, the shadow system or record can include data retrieved from the record objects of the data source provider's system of record. It should be appreciated that although FIG. 16 illustrates a shadow system of record including one or more shadow record objects that correspond to respective record objects of a corresponding system of record of the data source provider, the data processing system 9300 is configured to directly match the electronic activities of the data source provider to the record objects of the system of record 9360 without having to first match the electronic activity to a shadow record object of the shadow system of record 9330.

The data source provider 9350 can store electronic activity 9305(1)—electronic activity 9305(N) (generally referred to as electronic activity 9305) in the data source 9355. As described above, the electronic activities can include one or more forms of electronic activity, such as email or other forms of electronic communication. The data processing system 9300 can access or otherwise retrieve the electronic activity 9305 from the data source 9355. For example, the above-described electronic activity ingestor 205 can be configured to ingest electronic activities in a real-time or near real-time basis for accounts of one or more enterprises, organizations, companies, businesses, institutions or any other group associated with the data source providers. The electronic activity ingestor 205 can ingest electronic activities. For example, when a data source provider subscribes to a service provided by the data processing system 9300, the data source provider can provide access to electronic activities maintained by the data source provider by going through an onboarding process. That onboarding process can enable the data processing system 9300 to access electronic activities owned or maintained by the data source provider in one or more data sources 9355. For example, the data sources 9355 can be, but are not limited to, mail servers, one or more systems of record, one or more phone services or servers of the data source provider, among other sources of electronic activity. The electronic activities ingested during an onboarding process may include electronic activities that were generated in the past, perhaps many years ago, that were stored on the electronic activities' sources. The data processing system 9300 can be configured to ingest (and re-ingest) the electronic activities from one or more data sources 9355 on a periodic basis, including daily, weekly, monthly, or any reasonable frequency.

The data processing system 9300 can match the electronic activities 9305 to one or more record objects 1602 of the shadow system of record 9330. The record objects 1602 of the shadow system of record 9330 can be synced with the record object 1602 of the system of record 9360.

Syncing the shadow record objects 1602 with the record objects 1602 of the system of record 9360 can include adding values from fields of the shadow record objects 1602 to the corresponding values, such as matched electronic activities 9305, of the record objects 1602 in the system of record 9360. In some implementations, the data processing system 9300 can match the electronic activities 9305 directly to the system of record 9360. For example, the data processing system 9300 can match the electronic activities 9305 to the record objects in the system of record 9360 without matching the electronic activities 9305 to the record objects in the shadow system or record 9330.

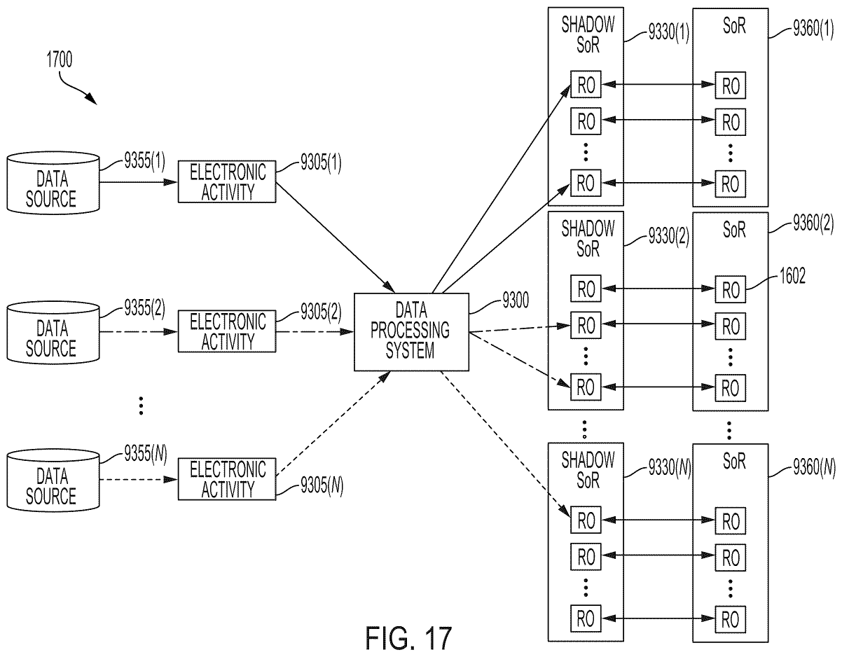

FIG. 17 illustrates a block diagram of an example process flow 1700 for processing electronic activities in a multi-tenant configuration. As illustrated by the process flow 1700, the multi-tenant configuration can include a plurality of data sources 9355(1)-9355(N), each of which can be a component of a respective data source provider 9350(1)-9350(N). The data processing system 9300 can receive or access electronic activities 9305 from each of the respective data sources 9355(1)-9355(N).

The data processing system 9300 can identify from which of the data sources 9355, each of the respective electronic activities 9305 were received and then match the electronic activities 9305 with one or more record objects 1602 associated with the data source provider 9355.

For example, and as illustrated in FIG. 17, the data source 9355(1) can be associated with the shadow system or record 9330(1), the data source 9355(2) can be associated with the shadow system or record 9330(2), and the data source 9355(N) can be associated with the shadow system or record 9330(N). The data processing system 9300 can match the electronic activity 9305(1), from the data source 9355(1), with two of the record objects 1602 in the shadow system of record 9330(1). The data processing system 9300 can match the electronic activity 9305(2), from the data source 9355(2), with two of the record objects 1602 in the shadow system of record 9330(2). The data processing system 9300 can match the electronic activity 9305(N), from the data source 9355(N), with one of the record objects 1602 in the shadow system of record 9330(N).

In some implementations, the data processing system 9300 can match the electronic activities 9305 directly to the system of records 9360. For example, the data processing system 9300 can match the electronic activities 9305 to the record objects in the system of record 9360 without matching the electronic activities 9305 to the record objects in the shadow systems or record 9330.

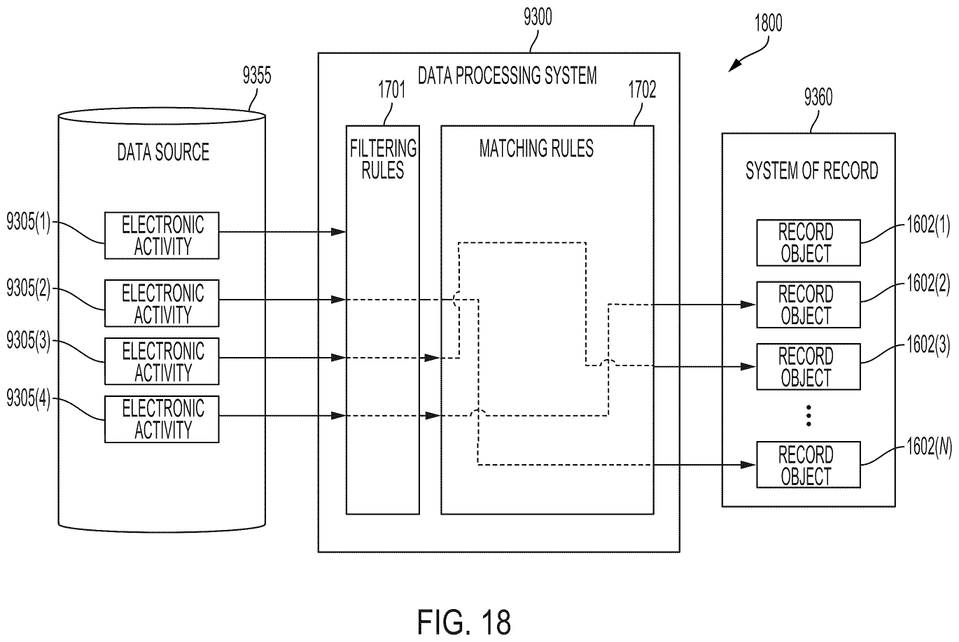

FIG. 18 illustrates a block diagram of an example process flow 1800 for matching electronic activities 9305 with record objects 1602. The data source 9355 includes a plurality of electronic activities 9305 that are accessed by or transmitted to the data processing system 9300. The data processing system 9300 can include a filtering rule set 1701 and matching rules 1702. The data processing system 9300 can use the filtering rule set 1701 and the matching rules 1702 to map the incoming electronic activities 9305 to one or more of the record objects 1602 in the system of record 9360.

Also, with reference to FIGS. 11 and 12, among others, the data processing system 9300 can include one or more filtering rule sets 1701. The filtering rule sets 1701 can include rule sets for filtering or excluding electronic activities 9305 from the matching process. For example, when the data processing system 9300 processes an incoming electronic activity 9305, the data processing system 9300 can first process the electronic activity 9305 with the filtering rule sets 1701 before attempting to match the electronic activity 9305 with a record object 1602. As illustrated in FIG. 18, the electronic activity 9305(1) can be received by the data processing system 9300. The data processing system 9300 can process the electronic activity 9305(1) with the filtering rule set 1701 before the data processing system 9300 passes the electronic activity 9305(1) to the matching rules 1702. As illustrated in FIG. 18, the electronic activity 9305(1) is processed with the filtering rule set 1701 and is restricted from further processing and is not matched with one of the record objects 1602.

The filtering rule set 1701 can include a plurality of rules or heuristics for determining whether the electronic activity 9305 should be restricted from further processing including matching the electronic activity to a record object. The rules can be keyword-based. For example, the rules can include a list of keywords. The data processing system 9300 can process the text of the electronic activity 9305 and determine whether one or more of the keywords are present in the electronic activity 9305. The data processing system 9300 can determine the electronic activity 9305 should be restricted if the data processing system 9300 identifies one of the keywords in the electronic activity 9305. The data processing system 9300 can identify identical matches of the keyword. The data processing system 9300 can identify approximate or fuzzy matching of the keyword (e.g., the data processing system 9300 can identify misspellings or plurals of the keyword). In some implementations, the keywords can include wildcards. For example, the keyword may be only the base or root of a word. The rules can be pattern-based. For example, the rules can include regex patterns with which the data processing system 9300 processes the text of the electronic activities 9305. For example, the regex pattern can be configured to identify social security numbers.

If the data processing system 9300 determines that the electronic activity 9305 is selected with one of the rules of the filtering rule set 1701, the data processing system 9300 can stop further processing or ingestion of the electronic activity 9305. For example, if the electronic activity 9305 is an email that includes a social security number and one of the rules of the filtering rule set 1701 is configured to identify social security number patterns, the data processing system 9300 can identify the email with the rule and stop ingestion of the email such that the email is not matched to one of the record objects 1602. The electronic activities 9305 identified by the filtering rule set 1701 can be ingested but are restricted from being matched to one or more record objects. For example, the electronic activity 9305 may be restricted from being matched to a record object, but the data processing system 9300 can use the data in the electronic activity 9305 to populate fields with values in the above-described node profile graph.

The data processing system 9300 can include one or more matching rules 1702. The rules of the matching rules 1702 can include rules for matching electronic activities 9305 with one or more record objects 1602. Also referring to FIGS. 11 and 12, among others, the rules for matching the electronic activities 9305 to record objects 1602 can be grouped into sets such as buyer-side rules or strategies that match electronic activities 9305 to record objects 1602 based on data related to the recipient of the electronic activity 9305. Another example filtering rule set 1701 can include a grouping of rules based on seller-side rules or strategies that match electronic activities 9305 to record objects 1602 based on data related to the sending of the electronic activities 9305. The data processing system 9300 can match the electronic activities 9305 to the record objects 1602 based on a plurality of matching rules 1702. For example, the electronic activity 9305(3) is matched with the record object 1602(3) based on a plurality of matching rules 1702(1). In some implementations, the matching rules 1702 can be to select a group of record objects. The data processing system 9300 can then select a candidate record object from an intersection of the groups of record objects. For example, the candidate record object may be the record object that is selected by each of the matching rules 1702.

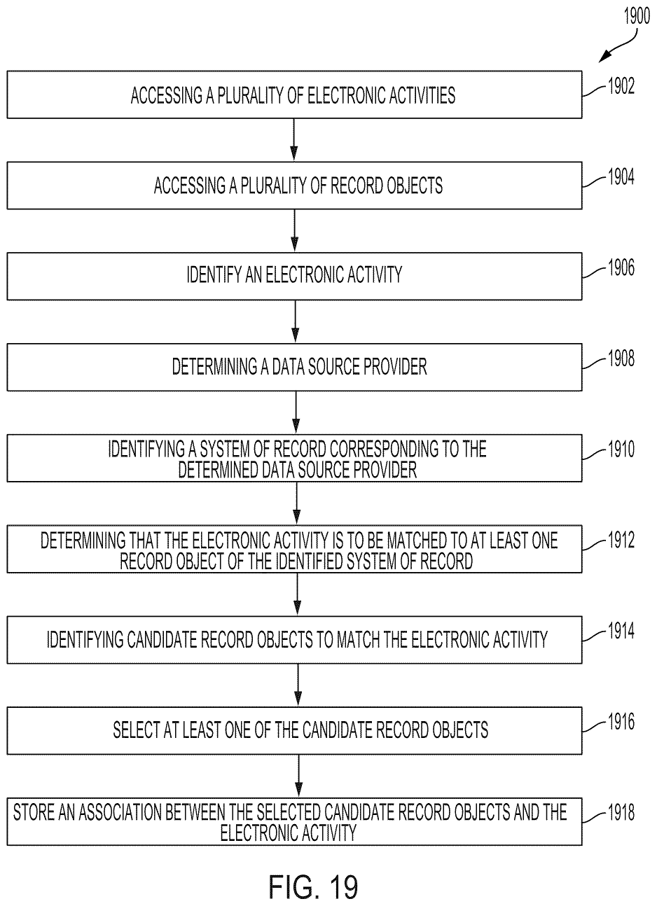

FIG. 19 illustrates a method 1900 to match electronic activities directly to record objects. The method 1900 can include accessing a plurality of electronic activities (BLOCK 1902). With reference to FIGS. 16-18, among others, the data processing system 9300 can access a plurality of electronic activities. The electronic activities can be transmitted to the data processing system 9300 from data source providers. The data processing system 9300 can retrieve the electronic activities from the data source providers. For example, the data source provider can include or be an email server. The data processing system 9300 can have the authority to access the emails stored on the email server through an API or an HTTP method (e.g., a GET method).

The method 1900 can include accessing a plurality of record objects (BLOCK 1904). The method 1900 can include accessing, by the data processing system 9300, a plurality of record objects. The data processing system 9300 can access the record objects for a plurality of different systems of record, as described above in relation to FIG. 17. For example, the data processing system 9300 can make a call to the systems of record 9360 that are associated with each of the data source providers from which the data processing system 9300 retrieved electronic activities at BLOCK 1902. The data processing system 9300 can generate a copy of the accessed record objects. The data processing system's copy of the access record objects can be referred to as shadow record objects.

As described above in relation to FIG. 10, each of the record objects can be of a record object type. For example, the record objects can be lead record objects, account record objects, opportunity record objects, or contact record objects. The record objects can be any type of record object in a system of record. The other systems of records can include Applicant Tracking Systems (ATS), such as Lever, located in San Francisco, CA or Talend by Talend Inc., located in Redwood City, CA, enterprise resource planning (ERP) systems, customer success systems, such as Gainsight located in Redwood City, CA, and Document Management Systems, among others.

The data processing system 9300 can retrieve the record objects from servers that correspond to the data source provider or data source from which the data processing system 9300 retrieved the electronic activities 9305. The data processing system 9300 can retrieve the record objects 1602 from a system of record 9360. The data processing system 9300 can retrieve the record objects 1602 through an API call. For example, the data processing system 9300 can retrieve a first plurality of record objects corresponding to a first system of record of a first data source provider and second plurality of record objects corresponding to a second system of record of a second data source provider.

As described above in relation to FIG. 17, among others, the system can be configured in a multi-tenant configuration. In a multi-tenant configuration, the data processing system 9300 can retrieve a respective plurality of record objects that correspond to each of the data source provider (e.g., tenants) associated with the data processing system 9300. For example, the data processing system 9300 can retrieve a plurality of record objects from a system of record for each of the data source providers.

Each of the record objects can include one or more object fields and corresponding object field values. For example, the record objects can be data structures and the object field values can be values of object fields of the data structure. For example, for a contact record object, the data structure can include fields such as, but not limited to, name, address, email, and phone number, which can be filled with respective field values.

The method 1900 can include identifying an electronic activity (BLOCK 1906). The method 1900 can include identifying, by the data processing system 9300, an electronic activity of the plurality of electronic activities to match to one or more record objects. The data processing system 9300 can identify an electronic activity that is a candidate for matching to one or more record objects. The data processing system 9300 can determine that an electronic activity is a candidate for matching to one or more record objects based on the filtering and exclusion rules. For example, if the electronic activity is identified by one or more filtering or exclusion rules the electronic activity can be disregarded from consideration for matching to a record object.

The data processing system 9300 can identify electronic activities as candidates based on one or more tags applied to the electronic activity. The above-described tagging engine 265 can assign one or more tags to the electronic activity when the electronic activity is ingested or processed. For example, if all the participants associated with the electronic activity are internal (e.g., each participant has an email address with the domain of the data source provider), the tagging engine 265 can tag the electronic activity as internal. The data processing system 9300 can be configured such that electronic activities tagged as internal are not matched to record objects. In another example, if the electronic activity includes a participant that is associated with an account record object, the data processing system 9300 can tag the electronic activity as a candidate for matching.

As described above in relation to FIGS. 5A-5C and 6B, the data processing system 9300 can identify and extract content from the electronic activities. For example, the data processing system 9300 can identify participants associated with the electronic activity. The participants can be the sender or the receiver of the electronic activity. The data processing system 9300 can identify the participants associated with the electronic activity by identify the sender's email address and the recipient's email address.

In some implementations, the data processing system 9300 can assign one or more tags to the electronic activity. The data processing system 9300 can assign tags to the electronic activities based on the content included in the electronic activity or the metadata therefor. For example, the tags can be based on one or more character strings identified in the body of the electronic activity, in the metadata of the electronic activity, or in related electronic activities.

For example, the electronic activity can be an email message and the data processing system 9300 can identify keywords within the email's body. The keywords can be identified by the above-described tagging engine 265. The keywords can identify the subject matter, phrases, accounts, topics, identification numbers, or other terms in or related to the subject of the electronic activity.

The method 1900 can include determining a data source provider (BLOCK 1908). The method 1900 can include the data processing system 9300 from which of the data source providers, the data processing system 9300 received the electronic activity. For example, the data processing system 9300 can receive electronic activity from a plurality of data source providers. In some implementations, when the data processing system 9300 receives the electronic activity, the electronic activity can label or store the electronic activity in a database in association of the data source provider that provided the electronic activity.

The method 1900 can include identifying a system of record (BLOCK 1910). The data processing system 9300 can identify a system of record that corresponds to the data source provider that the data processing system 9300 identified at BLOCK 1908. The data processing system 9300 can identify a plurality of candidate record objects that are associated with the data source provider. For example, and referring to FIGS. 3, 4, and 16-18, among others, once the data processing system 9300 identifies a system of record 9360, the data processing system 9300 can identify the record objects in the system of record 9360 as candidate record objects to which the electronic activity can be matched. The data processing system 9300 can match the electronic activity with one or more of the record objects in the system of record 9360.

In some implementations, the data processing system 9300 can identify the shadow record objects in the shadow system of record as candidate record objects. For example, and referring to FIG. 3, the system of record from each data source provider can be copied into the data processing system 9300 as shadow system of record 9330. Each of the shadow systems of record 9330 can include a plurality of record objects that are shadow record objects of the record objects in the data source provider's system of record 9360. The data processing system 9300 can match the electronic activity to one of the identified shadow record objects. The data processing system 9300 can directly match the electronic activity to one or more record objects in the shadow system of record 9330, one or more record objects in the system of record 9360, or one or more record objects in both the shadow system of record 9330 and the system of record 9360 subject to limitations of the system of record 9360. In some implementations, the data processing system 9300 can match the electronic activity to one or more record objects in the shadow system of record 9330, which can then be synced with the record objects in the system of record 9360. In some implementations, the data processing system 9300 can match the electronic activity to more than one record object in the shadow system of record 9330. In some such implementations, the data processing system 9300 can determine the shadow record object with which the electronic activity most closely matches (or has the highest match score) and cause the electronic activity to match the corresponding record object in the system of record 9360.

In some implementations, each of the electronic activities can be associated with a domain. For example, the domain can be identified by the sending email address of the electronic activity. The data processing system 9300 can identify the system of record based on a domain associated with an email address of the sender of the electronic activity.

The method 1900 can include determining whether the electronic activity can be matched to a record object (BLOCK 1912). The data processing system 9300 can determine if the electronic activity can be matched to a record object by applying a first policy. The policy can include one or more filtering rules.

For example, and also referring to FIGS. 4 and 18 among others, the filtering engine 270 can first process the electronic activity with filtering rules 1701 to determine whether the electronic activity should be blocked, removed from further processing, redacted, or deleted from the data processing system 9300.

The above described filtering engine can determine the electronic activity should not be matched to a record object based on one or more filtering rules. The filtering rules can restrict the data processing system 9300 from performing further processing or matching on the electronic activity. The filtering rules can include a keyword rule configured to restrict electronic activities including a predetermined keyword; a regex pattern rule configured to restrict electronic activities including one or more character strings that match a predetermined regex pattern; a logic-based rule configured to restrict electronic activities based on the participants of the electronic activities satisfying a predetermined group of participants; or any combination thereof.

The filtering rules can be defined by the data source provider of the electronic activity and the system of record to which to match the electronic activity. For example, the data source provider can define rules for electronic activities that should not be matched to the record objects in its system of record 9360.

In some implementations, the filtering engine 270 can restrict electronic activities from being matched to a record object by applying one or more rules to the electronic activity to identify the electronic activities that should not be matched with a record object. The rules can include determining that the electronic activity includes one or more predetermined words included in a list of restricted words. For example, electronic activities that include terms or phrases related to a specific product identified by the data source provider or department (e.g., legal department) associated with the data source provider can be identified by the filtering engine 270 for restriction from further processing.

In some implementations, the filtering engine 270 can restrict electronic activity from being matched with a record object if the electronic activity includes any character strings that has a regular expression pattern that matches a predefined regex pattern included in a list of restricted regex patterns. For example, the filtering engine 270 can include a list of restricted regex patterns that can include a pattern to identify social security numbers, bank account numbers, credit card numbers, dates of birth, or other sensitive information.

The filtering engine 270 can restrict electronic activity from being matched with a record object by determining that the sender of the electronic activity match a sender included in a list of restricted sender list. For example, the email address of the company's general counsel can be included on a restricted sender list and all of the emails sent by the general counsel will be restricted out by the filtering engine 270. The filtering engine 270 can restrict electronic activity from being matched with a record object by determining that a recipient of the electronic activity matches a recipient included in a restricted recipient list. For example, the filtering engine 270 may restrict out any email or electronic activity sent to a human resource manager. The filtering rules 1701 can include one or more rule sets. The rules in the filtering rules 1701 can be defined by the data processing system 9300. The rules can be global rules that the data processing system 9300 can apply to the electronic activities of each data source provider. The data processing system 9300 can include semi-global rules that are applied to the electronic activities from a subset of the data source providers. For example, the data processing system 9300 can have finance semi-global rules that are applied to the electronic activities from data source providers involved in the business of finance. The rules can be defined or otherwise configured by the data source provider and applied to only the electronic activities associated with the data source provider.

The filtering engine 270 can restrict electronic activity from being matched with a record object based on a sender-recipient pair. For example, the filtering engine 270 can include a restriction list that includes a plurality of sender-recipient pairs. When a sender of the electronic activity sends an electronic activity to one of the recipients with which the sender is paired in the restriction list, the filtering engine can restrict out the electronic activity.

If the filtering engine 270 does not restrict the electronic activity from further processing by identifying the electronic activity with the filtering rules 1701, the data processing system 9300 can determine that the electronic activity should be matched with one of the candidate record objects associated with the data source provider.

The method 1900 can include identifying candidate record objects (BLOCK 1914). The data processing system 9300 can identify one or more candidate record objects to which the electronic activity can be matched. For example, as described above in relation to FIG. 12, the electronic activity can be matched to a plurality of record objects. The data processing system 9300 can identify the candidate record objects based on applying a second policy. The second policy can include one or more rules for identifying candidate record objects based on one or more participants of the electronic activity.

Also referring to FIGS. 11, 12, and 18, among others, the data processing system 9300 can identify the plurality of record objects to which the electronic activity can be matched based on one or more rules or rule sets. The rules that identify to which of the record objects the data processing system 9300 can match the electronic activity can be included in a second policy that includes one or more rule sets. The data processing system 9300 can identify the plurality of record objects based on one or more tags assigned to the electronic activity by the tagging engine 265.

As described above, the electronic activity linking engine 250 can identify one or more candidate record objects to match the electronic activity using recipient-based rules that identify the candidate record objects based on one or more recipients of the electronic activity. The recipient-based rules can include rules for identifying the recipient based on a specific recipient (e.g., based on an email address). The recipient-based rules can include rules for identifying the recipient based on data associated with the recipient. For example, the rule can identify recipients having a predetermined domain in their email address. An indication of the recipient can be included in the identified record object as a value in an object field.

The electronic activity linking engine 250 can identify one or more candidate record objects to match the electronic activity using sender-based rules that can identify the candidate record objects based on the sender of the electronic activity. The sender-based rules can include rules for identifying the record object based on a specific sender or based on data associated with the sender. An identification of the sender can be included in the identified record object as a value in an object field.

In some implementations, the electronic activity linking engine 250 can identify the candidate record objects based on sender-based rules or recipient-based rules or both. For example, and referring to FIG. 11, the electronic activity linking engine 250 can select a first group of candidate record object using the recipient-based rules and a second group of candidate record objects using the sender-based rules. The electronic activity linking engine 250 can match the electronic activity to one of the candidate record objects that is included in the both the first group of record objects and the second group of record objects.

In some implementations, the matching rules can be configured to select record objects of a specific type. For example, and also referring to FIGS. 10-12, among others, the matching rules can include a first set of rules that identify account record objects, a second set of rules that identify opportunity record objects, and a third set of rules that identify lead record objects.

Each of the matching rules can have a priority level, score, or weight. The candidate record objects selected with rules with a higher priority level can be assigned a higher score. For example, if the rules select multiple record objects, the electronic activity linking engine 250 can select the candidate record object with the highest score. In some embodiments, a candidate record object can be selected multiple times. For example, a first and a second matching rule can each select a given record object. The record object selected by multiple matching strategies can be given an aggregate (for example, a weighted aggregate) of the scores associated with each of the matching rules that selected the candidate record object.

The data source provider can assign the priority level, score, rank, or weight to each of the matching rules. For example, the data source provider can assign a first priority level to a first subset of the matching rules and a second priority level to a second subset of the matching rules.

Also referring to FIGS. 5A-9, among others, the electronic activity linking engine 250 can identify candidate record objects based on matching rules that can identify record objects based on an object field value of the record object that identifies one or more nodes. One or more participants of the electronic activity can be used to select a node of a node graph.

In some implementations, the rules can candidate record objects based on participants that are linked to a record object. For example, an account record object can include an object field that can include a plurality of values. The object field values can identify nodes of a node graph. The data processing system 9300 can, using the matching rules, select contact record objects that are associated with identified nodes of the node graph. In some implementations, the candidate record objects can be identified based on one or more of the participants associated with the electronic activity being identified in the object field value.

The object field of the record object can identify an object owner or team, which can be user, contact, or team that is responsible for the account associated with a record object. Based on the values, the data processing system 9300 can identify a plurality of contact record objects that are associated with the object as candidate record objects.

The data processing system 9300 can identify candidate record objects based on one or more tags assigned to the electronic activity. The tagging engine 265 and the tagging of electronic activity is described above in Section G, among others. The tagging engine 265 can tag the electronic activity as specifically mentioning an account, product, contact, lead, or as including another predetermined character string. One or more of the rules can select candidate record objects based on the selecting record objects associated with the one or more tags of the electronic activity. For example, a predetermined account tag can be applied to an email if the body of the email includes an identification of the tag and the data processing system 9300 can identify the account record object associated with the account tag as a candidate record object. In another example, the electronic activity can be parsed and the term “renewal” can be identified in the electronic activity. A “renewal” tag can be applied to the electronic activity. A matching rule to select record objects based on tags can select a renewal record object opportunity with the electronic activity and identify the renewal record object opportunity as a candidate record object. As described above in relation to FIG. 12, an indication of each of the record objects identified by a matching rule can be stored in a record object array 1202.

The method 1900 can include selecting a record object (BLOCK 1916). Also referring to FIG. 12, among others, the data processing system 9300 can include identify candidate record objects to which the electronic activity can be matched. As illustrated in FIG. 12, the matching rules can identify more than one candidate record objects. The electronic activity linking engine 250 can select one or more of the candidate record objects with which to match the electronic activity.

The electronic activity linking engine 250 can select the one or more record objects from the plurality of candidate record objects based on the priority level used to select or identify each of the plurality of candidate record objects. For example, as described above in relation to FIG. 11, among others, each of the matching rules can have a priority level, score, or weight. The candidate record objects selected with rules with a higher priority level can be assigned a higher score. For example, if the rules select multiple record objects, the electronic activity linking engine 250 can select the candidate record object with the highest score. In some embodiments, a candidate record object can be selected multiple times. For example, a first and a second matching rule can each select a given record object. The record object selected by multiple matching strategies can be given an aggregate (for example, a weighted aggregate) of the scores associated with each of the matching rules that selected the candidate record object.

The data source provider can assign the priority level, score, rank, or weight to each of the matching rules. For example, the data source provider can assign a first priority level to a first subset of the matching rules and a second priority level to a second subset of the matching rules.

The method 1900 can include storing an association between the selected candidate record object and the electronic activity (BLOCK 1918). For example, the data processing system 9300 can store, in a data structure, an association between the selected candidate record objects and the electronic activity. Also referring to FIGS. 3 and 4, among others, the electronic activity can be matched to one or more candidate record objects that are record objects in a shadow system of record for the data provider that provided the electronic activity.

In some implementations, once the electronic activity is matched with one or more record objects, the data processing system 9300 can identify subsequent electronic activities that are related to the matched electronic activities. For example, the data processing system 9300 can identify emails that are part of the same email chain. The data processing system 9300 can match each of the emails in the email chain to the one or more record objects to which the first email was matched.

In some implementations, the electronic activity linking engine 250 can detect changes in the stored associations between electronic activities and record objects. Once the electronic activity is matched to a record object a user can accept, reject, or update the linking between the electronic activity and the matched record object. The user can manually remap the linking of the electronic activity from a first record object to a second, different record object. In another example, the data processing system 9300 may automatically rematch electronic activities at predetermined intervals or when the data processing system 9300 receives additional data.

In some implementations, when the electronic activity linking engine 250 determines that the electronic activity is matched with a second, different record object, the electronic activity linking engine 250 can update the matching rules or policies that matched the electronic activity to the original record object. The electronic activity linking engine 250 can update the matching rules or policies such that the subsequent electronic activities are correctly matched with the correct record object.

16. Matching Electronic Activities to Record Objects of Systems of Record with Node Profiles

As described above, the system described herein can match electronic activities with one or more record objects. The system can match the electronic activities in a single-tenant or multi-tenant configuration of the system. For example, in a single-tenant configuration, the system can receive or access electronic activities from a single data source provider and match the electronic activities to record objects of a system of record of the data source provider from which the electronic activities were received or accessed. In a multi-tenant configuration, the system can receive or access electronic activities from multiple data source providers and match the electronic activities to record objects of a system of record of the respective data source provider from which the electronic activities were received or accessed. As described herein, the system can automatically match, link, or otherwise associate the electronic activities with one or more record objects. In some implementations, the system can match the electronic activity with one or more node profiles. The system can use the node profiles to identify one or more record objects with which the electronic activity can be matched. If the system determines the electronic activity is eligible or qualifies to be matched with one or more record objects, the system can match the electronic activity to one or more of the record objects identified with the node profiles using one or more set of rules or rule sets. The system can then rank the identified candidate record objects to select one or more record objects with which to associate the electronic activity. The system can then store an association between the electronic activity and the selected one or more record objects.

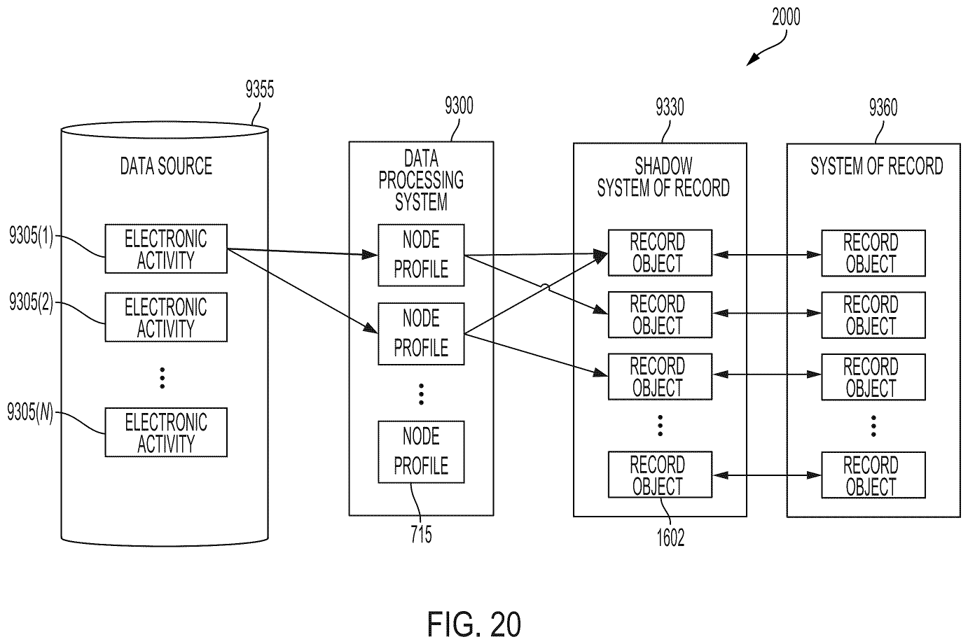

FIG. 20 illustrates a block diagram of an example process flow 2000 for processing electronic activities. Also, with reference to FIGS. 3, 4, and 16-18, among others, the data processing system 9300 can be in communication with one or more data source providers 9350. Each of the data source providers 9350 can include a data source 9355. FIG. 20 illustrates an example of a single-tenant system where the electronic activities 9305 from a single tenant (e.g., the data source provider 9350 that includes the data source 9355) is matched to the record objects 1602 of a single shadow system of record 9330. The single shadow system of record 9330 can be associated with the data source provider 9350 that provided the electronic activity. For example, the shadow system or record can include data retrieved from the record objects of the data source provider's system of record.

As illustrated in FIG. 17, among others, the system illustrated in FIG. 20 can be a multi-tenant system that can include a plurality of data sources 9355 that can each include a plurality of electronic activities 9305. The system can match the electronic activities with record objects in shadow systems of record 9330 or directly with systems of record 9360 associated with the respective data source 9355.

The data source provider 9350 can store electronic activity 9305(1)—electronic activity 9305(N) (generally referred to as electronic activity 9305) in the data source 9355. As described above, the electronic activities can include one or more forms of electronic activity, such as email or other forms of electronic communication. The data processing system 9300 can access or otherwise retrieve the electronic activity 9305 from the data source 9355. For example, the above-described electronic activity ingestor 205 can be configured to ingest electronic activities in a real-time or near real-time basis for accounts of one or more enterprises, organizations, companies, businesses, institutions or any other group associated with the data source providers. The electronic activity ingestor 205 can ingest electronic activities. For example, when a data source provider subscribes to a service provided by the data processing system 9300, the data source provider can provide access to electronic activities maintained by the data source provider by going through an onboarding process. That onboarding process can enable the data processing system 9300 to access electronic activities owned or maintained by the data source provider in one or more data sources 9355. For example, the data sources 9355 can be, but are not limited to, mail servers, one or more systems of record, one or more phone services or servers of the data source provider, among other sources of electronic activity. The electronic activities ingested during an onboarding process may include electronic activities that were generated in the past, perhaps many years ago, that were stored on the electronic activities' sources. The data processing system 9300 can be configured to ingest (and re-ingest) the electronic activities from one or more data sources 9355 on a periodic basis, including daily, weekly, monthly, or any reasonable frequency.

The data processing system 9300 can match the electronic activities 9305 with one or more node profiles 715. For example, and also referring to FIGS. 3-9, among others, the node graph generation system 200 can generate a node graph that includes a plurality of nodes. Each of the nodes can include a node profile 715, which can be a data structure that includes a plurality of fields. For example, an example node profile 715 can include fields such as, but not limited to name, email, phone, company, and job title. The system can ingest electronic activities and populate the fields with values. Also referring to FIG. 4, among others, as the system ingests additional emails the node profile manager can update the node profile 715. The node profile managed can update the node profile 715 by, for example, increasing or decreasing a confidence score of the values of fields that can be verified or contradicted by subsequent electronic activities. The node profile manager can add additional (e.g., updated) values to a field based on ingested electronic activities.

When matching an ingested electronic activity 9305 to a record object 1602, the data processing system 9300 can match the electronic activity 9305 with one or more node profiles 715. For example, the data processing system 9300 can identify or parse the sender and recipient email addresses from an email (an example electronic activity) and identify a first node profile 715 based on the sender's email address and a second node profile 715 based on the recipient's email address. As illustrated in FIG. 20, the electronic activity 9305(1) can be matched with a first and a second node profile 715. For example, one of the node profiles 715 can be associated with the sender and one of the node profiles 715 can be associated with the recipient of the electronic activity 9305(1). Additional details relating to matching electronic activities to node profiles are described herein in Section 17 and the descriptions above with respect to FIGS. 2-9.

The data processing system 9300 can match the electronic activities 9305 to one or more record objects 1602 of the shadow system of record 9330 using the node profiles 715 to which the electronic activity 9305 was matched. For example, and also referring to FIG. 6A, among others, the data processing system 9300 can use one or more values 620 from one or more fields 610 to identify candidate record objects. In some implementations, the node profiles 715 can include additional information that isn't extracted from the given electronic activity 9305 being matched to a record object. In this example, matching the electronic activity to a node profile 715 can enable the identification of additional record objects that may not be identified when using only data extracted from the electronic activity 9305. As illustrated in FIG. 20, a first node profile 715 is matched to a first and second record object 1602 and a second node profile 715 is matched to the first and a third record object 1602. Each of the node profiles 715 that are matched with a given electronic activity 9305 can match to the same record objects, different record objects, or first and second sets of record objects that at least partially intersect with one another.

The record objects 1602 of the shadow system of record 9330 can be synced with the record object 1602 of the system of record 9360. Syncing the shadow record objects 1602 with the record objects 1602 of the system of record 9360 can include adding values from fields of the shadow record objects 1602 to the corresponding values, such as matched electronic activities 9305, of the record objects 1602 in the system of record 9360. In some implementations, the data processing system 9300 can match the electronic activities 9305 directly to the system of record 9360. For example, the data processing system 9300 can match the electronic activities 9305 to the record objects in the system of record 9360 without matching the electronic activities 9305 to the record objects in the shadow system or record 9330.

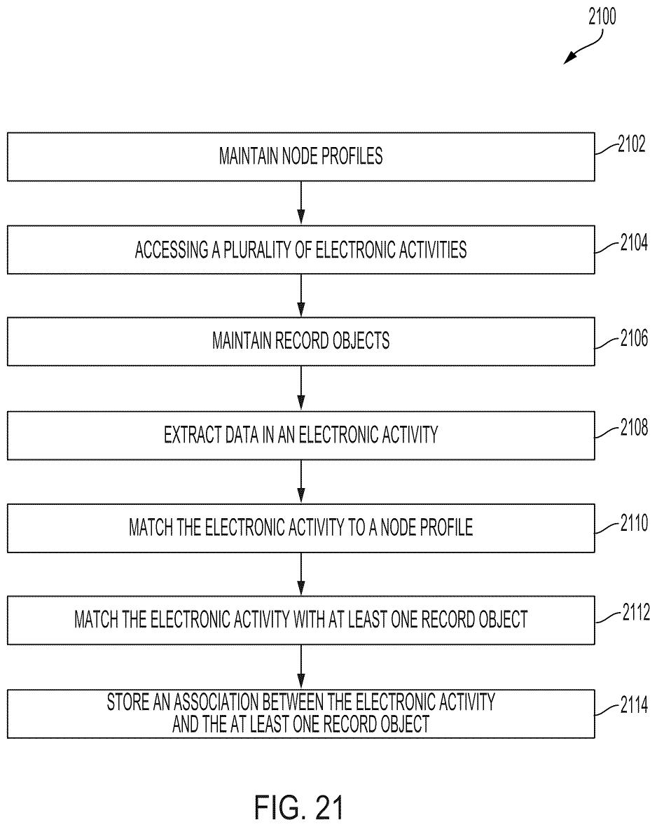

FIG. 21 illustrates a block diagram of an example method 2100 to match electronic activities to record objects of systems of record with node profiles. The method 2100 can include maintaining a plurality of node profiles (BLOCK 2102). The method 2100 can include maintaining, by one or more processors of the data processing system 9300, a plurality of node profiles. Also referring to FIG. 6A, among others, each of the node profiles can correspond to a different unique entity, such as a person or company. Each of the node profiles can include a plurality of fields, such as, but not limited to name, email address, company, domain, telephone number. Each of the fields can include one or more value data structures. Each of the value data structures can include node field value and one or more entries corresponding to respective data points that support the node field value of the value data structure. For example, and also referring to FIG. 6A among others, value data structure 615 can include a value 620, an occurrence metric 625, a confidence score 630 and one or more entries 635a-n. The entries 635 can include data (or an indication thereof) the basis for the value 620, the occurrence metric 625, and the confidence score 630.

For example, each entry 635 can identify a data source 640 from which the value was identified (for instance, a source of a system of record or a source of an electronic activity), a number of occurrences of the value that appear in the electronic activity, a time 645 associated with the electronic activity (for instance, at which time the electronic activity occurred) and an electronic activity unique identifier 502 identifying the electronic activity. In some embodiments, the occurrence metric 625 can identify a number of times that value is confirmed or identified from electronic activities or systems of record. The node profile manager 220 can be configured to update the occurrence metric each time the value is confirmed. In some embodiments, the electronic activity can increase the occurrence metric of a value more than once. For instance, for a field such as name, the electronic activity parser can parse multiple portions of an electronic activity. In some embodiments, parsing multiple portions of the electronic activity can provide multiple confirmations of, for example, the name associated with the electronic activity.

The method 2100 can include accessing a plurality of electronic activities (BLOCK 2104). The method 2100 can include accessing, by the one or more processors, a plurality of electronic activities transmitted or received via electronic accounts associated with one or more data source providers. The data processing system 9300 can update the node profiles using the electronic activities. With reference to FIGS. 16-20, among others, the data processing system 9300 can access a plurality of electronic activities. The electronic activities can be transmitted to the data processing system 9300 from data source providers. The data processing system 9300 can retrieve the electronic activities from the data source providers. For example, the data source provider can include or be an email server. The data processing system 9300 can have the authority to access the emails stored on the email server through an API or an HTTP method (e.g., a GET method).

As described herein in relation to FIGS. 4-8, among others, the data processing system 9300 can update the node profiles based on the accessed electronic activities. For example, the node profile manager 220 can maintain a node profile 715 for each unique entity, such as a person or company. As the data processing system 9300 ingests electronic activities, the node profile manager can update the node profile 715. The node profiles can be updated by changing one or more confidence scores of respective values corresponding to respective value data structures by adding additional data points to the value data structure that support the corresponding value. Furthermore, if a particular value of a field of a node profile doesn't exist, the node profile manager can add one or more additional values and corresponding value data structures to the field. The increase or decrease in the confidence score of values of fields can be based on the electronic activity. For example, when an electronic activity, such as an email is successfully transmitted to the intended destination, the node profile manager 220 can update the confidence score of the recipient email value. The data processing system 9300 can determine the email was successfully transmitted to the recipient, for example, if a bounce back email is not received in response to the email.

The method 2100 can include maintain one or more record objects (BLOCK 2106). The method 2100 can include maintaining, by the one or more processors, a plurality of record objects for one or more systems of record. Each of the record objects of the plurality of record objects can include one or more object fields having one or more object field values. As described above in relation to FIG. 20, among others, the data processing system 9300 can make a call to the systems of record 9360 that are associated with each of the data source providers from which the data processing system 9300 retrieved electronic activities. The data processing system 9300 can generate a copy of the accessed record objects. The data processing system's copy of the access record objects can be referred to as shadow record objects. The data processing system 9300 can update the shadow record objects and sync the changes back to the record objects in the tenant's system of record.

As described above in relation to FIG. 10, each of the record objects can be of a record object type. For example, the record objects can be lead record objects, account record objects, opportunity record objects, or contact record objects. The record objects can be any type of record object in a system of record. The other systems of records can include Applicant Tracking Systems (ATS), such as Lever, located in San Francisco, CA or Talend by Talend Inc., located in Redwood City, CA, enterprise resource planning (ERP) systems, customer success systems, such as Gainsight located in Redwood City, CA, and Document Management Systems, among others.

The data processing system 9300 can retrieve the record objects from servers that correspond to the data source provider or data source from which the data processing system 9300 retrieved the electronic activities 9305. The data processing system 9300 can retrieve the record objects 1602 from a system of record 9360. The data processing system 9300 can retrieve the record objects 1602 through an API call. For example, the data processing system 9300 can retrieve a first plurality of record objects corresponding to a first system of record of a first data source provider and second plurality of record objects corresponding to a second system of record of a second data source provider.

As described herein the system can be configured in a multi-tenant configuration or a single-tenant configuration. In a multi-tenant configuration, the data processing system 9300 can retrieve a respective plurality of record objects that correspond to each of the data source provider (e.g., tenants) associated with the data processing system 9300. For example, the data processing system 9300 can retrieve a plurality of record objects from a first system of record and from a second system of record.

Each of the record objects can include one or more object fields and corresponding object field values. For example, the record objects can be data structures and the object field values can be values of object fields of the data structure. For example, for a contact record object, the data structure can include fields such as, but not limited to, name, address, email, and phone number, which can be filled with respective field values.

The method 2100 can include extracting data from an electronic activity (BLOCK 2108). The method 2100 can include extracting, by the one or more processors, data included in an electronic activity of the plurality of electronic activities. For example, and referring to FIGS. 5A-5C among others, the data processing system 9300 can one or more recipients 510, one or more senders 512 of the electronic activity. The data processing system 9300 can identify a subject line 514, an email body 516, an email signature 518, and a message header 520 of the electronic activity. The message header can include additional information relating to the transmission and receipt of the email message, including a time at which the email was sent, a message identifier identifying a message, an IP address associated with the message, a location associated with the message, a time zone associated with the sender, a time at which the message was transmitted, received, and first accessed, among others. The electronic message 505 can include additional data in the electronic message 505 or in the header or metadata of the electronic message 505. In some implementations, the electronic activity can be an email, a call entry, a calendar entry, among others.

The method 2100 can include matching the electronic activity to a node profile (BLOCK 2110). The method 2100 can include matching, by the one or more processors, the electronic activity to at least one node profile of the plurality of node profiles. The data processing system 9300 can match the electronic activity to the one or more node profiles based on determining that the extracted data of the electronic activity and the one or more values of the fields of the at least one node profile satisfy a node profile matching policy. For example, as described herein, each value in a value data structure can include a confidence score.

In some implementations, the data processing system 9300 can identify a sender and one or more recipients of the electronic activity. For example, the data processing system 9300 can extract from the electronic activity the sender's email address and the email addresses of the one or more recipients of the electronic activity. The data processing system 9300 can match the electronic activity to a plurality of node profiles. For example, the data processing system 9300 can match the electronic activity to a first node profile based on the sender's email address. The data processing system 9300 can also match the electronic activity to one or more additional node profiles based on the extracted recipient email addresses. In some embodiments, strings or values are extracted from electronic activities and associated with candidate or potential fields to form field-value pairs. These field-value pairs extracted from an electronic activity can then be compared with corresponding field-value pairs of node profiles to identify or compute a match score between the electronic activity and respective node profiles having the field-value pairs with which the field-value pairs of the electronic activity are compared.

In some implementations, the matching policies for the matching of the electronic activity to one or more node profiles can be based on tags associated with the electronic activity. For example, the data processing system 9300 can determine a relationship between two or more node profiles based on the one or more values of the fields of the two or more node profiles. The data processing system 9300 can assigning one or more tags to the electronic activity based on the relationship between the two or more node profiles. In one example, the data processing system 9300 can assign a personal tag to the electronic activity. For example, the node profile manager 220 can be configured to determine that two node profiles have a personal (non-professional) relationship based on the electronic activities exchanged between the users associated with the node profiles and apply a “personal” tag to the emails between the users. The system can further determine a confidence score for the tag classifying the two node profiles based on how confident the system is in the prediction that the two node profiles have a personal relationship. In some embodiments, the node profile manager 220 can further determine if two nodes have a personal relationship based on commonalities in values in their node profiles, for instance, their home addresses (if they are neighbors), college or school affiliations (alumni/classmates), same last names, other non-professional affiliations, or other signals that may indicate the two node profiles may have a personal relationship. In some implementations, the data processing system 9300 can determine to not match an electronic activity that is associated with a personal tag to one or more record objects.

The data processing system 9300 can assign one or more tags to the electronic activity based on one or more policies. The data processing system 9300 assign the tags based on one or more node profiles associated with a sender or one or more recipients of the electronic activity. For example, the data processing system 9300 can identify, based on the body of the electronic activity, that the electronic activity is related to a sales deal and can tag the electronic activity with a sales tag. The data processing system 9300 can assign tags based on a relationship between the one or more node profiles associated with the sender and the one or more recipients of the electronic activity. For example, as described herein the data processing system 9300 can determine whether the users associated with the node profiles have a professional or personal relationship and assign a professional or personal tag to the electronic activity, accordingly. The data processing system 9300 can assign tags to the electronic activity based on one or more strings identified in the electronic activity. For example, the data processing system 9300 can parse the body of the email with regex to identify an account number and the data processing system 9300 can assign a tag based on the account number. The data processing system 9300 can assign the tags to the electronic activity based on one or more strings identified in the metadata of the electronic activity. For example, the metadata can be a header of an email and can include a domain associated with the sender of the electronic activity and the data processing system 9300 can assign a tag based on the domain identified in the header of the email.

The data processing system 9300 can match the electronic activity to one or more node profiles based on contribution scores. For example, each data point for a value in a value data structure can include a contribution score. The contribution score can indicate the contribution of the data point to the value. The data point's contribution score can be time dependent. For example, as described in relation to FIG. 7, among others, the contribution of the data point can decrease over time. In one example, a data point can have a greater contribution score if the data point was recently updated or generated when compared to a data point that was updated or generated in the past. Based on the contribution scores for each of the data points associated with the value, the data processing system 9300 can calculate a confidence score of the value of the field of the node profile. The data processing system 9300 can select the node profiles based on the confidence scores. For example, the electronic activity may match to a plurality of node profiles based on a value of a field in each of the node profiles. The data processing system 9300 can discard each of the node profiles as candidate node profiles if the value of the field in a node profile has a confidence score below a predetermined threshold.

In some implementations, the contribution score of the data point can be based on a trust score associated with the data source provider. The data processing system 9300 can determine, for a data point, a contribution score for the data point based on the trust score associated with the data source provider. For example, a relatively low trust score can reduce the confidence score of the data point.

The trust score can be based on a type of source of the data point.

The method 2100 can include matching the electronic activity to one or more record objects (BLOCK 2112). The method 2100 can include matching, by the one or more processors, the electronic activity to at least one record object of the plurality of record objects based on the extracted data of the electronic activity and object values of the at least one record object. For example, the data processing system 9300 can identify a sender of the electronic activity. The data processing system 9300 can select a first node profile of the plurality of node profiles based on the sender of the electronic activity. For example, the data processing system 9300 can identify the email address of the sender and select a node profile based on the identified email address. Based on the node profile, the data processing system 9300 can identify a first set of record objects of the plurality of record objects. For example, and also referring to FIG. 12, among others, the data processing system 9300 can identify a plurality of account, opportunity, and lead record objects based on the email address of the sender. The data processing system 9300 can also identify one or more record objects based on node field values contained in the identified node profile. For example, the node profile can include a field for teams on which the user is assigned. The field can include a value in a value data structure for each of the teams on which the user is assigned. The data processing system 9300 can select one or more record objects based on the teams identified in the node profile.

The data processing system 9300 can identify one or more record objects based on a recipient of the electronic activity. For example, the data processing system 9300 can identify a recipient email address of the electronic activity. The data processing system 9300 can identify a second node profile of the plurality of node profiles based on the recipient of the electronic activity. The data processing system 9300 can identify a second set of record objects of the plurality of record objects based on the second node profile.

In some implementations, the data processing system 9300 can filter or prune the first set of record objects (e.g., the record objects selected based on the sender of the electronic activity) and the second set of record objects (e.g., the record objects selected based on the recipient of the electronic activity). For example, the data processing system 9300 can identify an intersection of the first and second set of record objects (e.g., record objects that are included in both the first and second set of record objects). The data processing system 9300 can match the electronic activity to at least one of the record objects in the intersection of the first set of record objects and the second set of record objects.

In some implementations, each of the record objects in the intersection of the first set of record objects and the second set of record objects can be referred to as candidate record objects. The candidate record objects can include one or more types of record object types. For example, the record objects can be account record objects, opportunity record objects, or lead record objects, among others. The data processing system 9300 can match the electronic activity with one or more of the record objects in candidate record objects. For example, the data processing system 9300 can match the electronic activity to record objects in the candidate record objects that have different types. For example, the data processing system 9300 can match the electronic activity to an account record object and an opportunity record object. In some implementations, the data processing system 9300 can match the electronic activity to multiple record objects with the same type. For example, the data processing system 9300 can match the electronic activity to two candidate record objects that are both account record objects.

The method 2100 can include matching the electronic activity to one or more of the identified record objects based on one or more matching policies, rules, heuristic, or filters. The matching policies can be based on the sender and/or recipient of the electronic activity. The data processing system 9300 can identify a first set of matching policies based on the sender of the electronic activity and a second set of matching polices based on the recipients of the electronic activity. As described herein, the data processing system 9300 can identify a first set of candidate record objects based on the first set of matching policies and a second set of candidate record objects based on the second set of matching policies. The data processing system 9300 can identify an intersection between the first and second sets of candidate record objects.

For example, and also referring to FIGS. 4 and 18 among others, the filtering engine 270 can first process the electronic activity with filtering rules 1701 to determine whether the electronic activity should be blocked, removed from further processing, redacted, or deleted from the data processing system 9300. The above described filtering engine can determine the electronic activity should not be matched to a record object based on one or more filtering rules. The filtering rules can restrict the data processing system 9300 from performing further processing or matching on the electronic activity. The filtering rules can include a keyword rule configured to restrict electronic activities including a predetermined keyword; a regex pattern rule configured to restrict electronic activities including one or more character strings that match a predetermined regex pattern; a logic-based rule configured to restrict electronic activities based on the participants of the electronic activities satisfying a predetermined group of participants; or any combination thereof.

The data processing system 9300 can also use one or more policies (e.g., matching rules 1702) to select the candidate record objects. matching policies can be defined by the data source provider of the electronic activity and the system of record to which to match the electronic activity. For example, the data source provider can define rules for electronic activities that should not be matched to the record objects in its system of record 9360. The rules can include determining that the electronic activity includes one or more predetermined words included in a list of restricted words. For example, electronic activities that include terms or phrases related to a specific product identified by the data source provider or department (e.g., legal department) associated with the data source provider can be identified by the filtering engine 270 for restriction from further processing.

In some implementations, the data processing system 9300 can identify, using natural language processing, a string in the electronic activity. The matching policies can include matching the electronic activity to one or more record objects based on the string identified in the electronic activity. For example, the data processing system 9300 can identify a string in the body of the electronic activity. The data processing system 9300 can identify the string using regex or other pattern matching technique. The string can include an account number or other identifier. The data processing system 9300 can select the candidate record objects based on the string in the body of the electronic activity.

In some implementations, the matching policies can match the electronic activity to one or more record objects based on tags associated with the electronic activity. The data processing system 9300 can identify a first subset of record objects based on one or more tags assigned to the electronic activity. The data processing system 9300 can then match the electronic activity to at least one record object in the first subset of the record objects based on the one or more tags assigned to the electronic activity.

The method 2100 can include storing an association between the electronic activity and one or more record objects (BLOCK 2114). The method 2100 can include storing the association in a data structure. Also referring to FIGS. 3 and 4, among others, the electronic activity can be matched to one or more candidate record objects that are record objects in a shadow system of record (or the system of record) for the data provider that provided the electronic activity.

In some implementations, once the electronic activity is matched with one or more record objects, the data processing system 9300 can identify subsequent electronic activities that are related to the matched electronic activities. For example, the data processing system 9300 can identify emails that are part of the same email chain. The data processing system 9300 can match each of the emails in the email chain to the one or more record objects to which the first email was matched.

In some implementations, the data processing system 9300 can detect changes in the stored associations between electronic activities and record objects. Once the electronic activity is matched to a record object a user can accept, reject, or update the linking between the electronic activity and the matched record object. The user can manually remap the linking of the electronic activity from a first record object to a second, different record object. In another example, the data processing system 9300 may automatically rematch electronic activities at predetermined intervals or when the data processing system 9300 receives additional data.

As described herein, and in relation to the stage classification engine 325, for example, once one or more electronic activities are matched to a record object (e.g., an opportunity record object), the data processing system 9300 can classify a stage of the record object. The stages can be a stage, step, or task in a business process, a sales process, a hiring process, a support ticket, or other workflow. The stages can be defined by the system or by the data source provider. For example, the data processing system 9300 can identify at least a subset of the plurality of electronic activities that are matched to a first record object. The data processing system 9300 can also identify, for each of the electronic activities matched with the record object, one or more node profiles. The data processing system 9300 can determine a stage of the first record object based on the identified one or more node profiles of each of the subset of electronic activities.

Using the example of an opportunity record object in a sales process, the stages can indicate the steps taken in an opportunity or deal from the beginning of the deal to the final disposition of the deal (e.g., close and won or closed and lost). The stages can include, but are not limited to: prospecting, developing, negotiation, review, closed/won, or closed/lost.

In some implementations, the stages can be based on the contacts present or involved on one or more sides of the deal. For example, as the deal advances to higher stages, more senior people may be included in the electronic activities. The stage of the deal can be based on the identification or introduction of the above-described OCR. The data processing system 9300 can identify the OCR or other contacts present or involved on the deal based on the node profiles. For example, the data processing system 9300 can identify the node profiles based matched with the one or more electronic activities associated with a record object. Based on the node profiles, the data processing system 9300 can determine each of the contacts roles, positions, or titles. For example, “title” can be one of the fields in the node profile. The data processing system 9300 can use the field node value in the title field to determine the title of the person involved with the record object. The data processing system 9300 can also determine the stage of the record object based on the one or more tags assigned to the electronic activity associated with the record object.

In some implementations, the data processing system 9300 can maintain a normalized set of stages. The normalized set of stages can be referred to as processor assigned stages. Each of the data source providers can define custom stages for the record objects of the data source provider. Each stage (of the processor assigned stages or the data source provider assigned stages) can indicate a proximity to the completion of an event, task, process, or other workflow. The data processing system 9300 can generate a mapping between the data source provider assigned stages and the processor assigned stages. For example, the stage classification engine 325 can define five, normalized stages. A first data source provider can define a deal or opportunity as including 7 stages. A second data source provider can define a deal or opportunity as including 3 stages. The stage classification engine 325, for the first data source provider, may map stages 1 and 2 to normalized stage 1, stage 3 to normalized stage 2, stage 4 to normalized stage 3, stage 5 to normalized stage 4, and stages 6 and 7 to normalized stage 5. Accordingly, the data source provider's stages can be mapped to the normalized stages based on the tasks, requirements, or content of the stages rather than by the naming or numbering of the stages.

### 17. Linking Electronic Activities to Node Profiles

The present solution can enable real-time or near real-time linking of electronic activities to node profiles, with increased accuracy. In some systems that maintain data regarding entities, such as individuals or enterprises, including systems of record, the data may be self-reported, such as in response to specific queries to provide data for fields such as first name, last name, title, or email. As such, this data may be inaccurate. For example, when the data was provided, the data may have been inaccurate due to the data being self-reported. At a particular instant in time after the data was provided, due to changes to the data that may have occurred subsequent to when the data was provided and before the data has been updated, the data even if it was previously correct at the time the data was provided, may also eventually become obsolete, stale or inaccurate.

The present solution described herein can match electronic activities to node profiles maintained by a node graph generation system, that can use the data included in the electronic activities to update node profiles and the values of fields of these node profiles unobtrusively and without requiring any human input. As such, the present disclosure describes solutions for maintaining node profiles that remain accurate as the node profiles do not include self-reported information submitted by a user to update the node profile and because the node profiles are automatically updated as electronic activities are ingested and processed by the system without requiring any human activity. In this way, the present solution can enable dynamic updates to node profiles and a node graph including such node profiles, rather than manual/self-reported updates.

By linking electronic activities to node profiles, the present solution can increase the accuracy and validity of the node profiles, such as by increasing a likelihood that each node profile represents the true state of the world. For example, when node profiles are used to generate a node graph indicative of a hierarchy or other relationships amongst node profiles, the present solution can more accurately represent values of fields such as hierarchical titles within enterprises that are used to generate the node graph. The present solution can more accurately rank each value of each field (each value representing a potential true state of the world) by dynamically updating the confidence score corresponding to each value responsive to extracting data from electronic activities, so that the present solution outputs an evidence-based estimation of which value is the true value with improved accuracy. As an example, a node profile can include a first email address corresponding to a first enterprise at which the user corresponding to the node profile was employed and a second email corresponding to a subsequent enterprise at which the user corresponding to the node profile was employed. Each of the two email addresses are at respective points in time, accurate and valid. As the person switches jobs, the first email address is no longer valid but the confidence score associated with the first email address can in some embodiments, remain high indicating that the first email address belongs to the node profile. Similarly, the second email address also belongs to the node profile and therefore also can have a confidence score that may start low but increase as more electronic activities including the second email address are processed by the data processing system described herein. After the system determines that the second email address is active and functioning, the system can automatically increase the confidence score of the second email address since the contribution scores provided by recent data points (for example, recent electronic activities identifying the second email address) can contribute towards the higher confidence score while automatically decreasing the confidence score of the first email address since the electronic activities supporting the first email address are getting older and no new electronic activities serve as data points for the first email address. The present solution can thus respond to changes in the true state of the world represented by the node profile using the second email, rather than relying on self-reported information which may be inaccurate and/or delayed.

Referring further to FIG. 2, among other, the node graph generation system 200 can ingest electronic activities to generate or update node profiles that are maintained by the node graph generation system 200 using data from the electronic activities. For example, as illustrated in FIGS. 5A-5C and 6B, the node graph generation system 200 can process electronic activities such as an email 505, a call entry 525, or a calendar entry 560. For example, the node graph generation system 200 can process the email 505 to identify a plurality of strings having data from the To: field 510 (to identify a recipient of the email 505), the From: field 512 (to identify a sender of the email 505), the email body 516 (to identify a recipient of the email 505), and the email signature 518 (to identify the sender of the email 505).

Using the identified plurality of strings, the node graph generation system 200 can generate activity field-value pairs. Each activity field-value pair can include a data structure that associates a particular field to a value for the field that the node graph generation system 200 extracts from the electronic activity. For example, the node graph generation system 200 can generate a FirstName-value pair associating a value of “John” to the first name field, a LastName-value pair associating a value of “Smith” to the last name field, a Title-value pair associating a value of “Director” to the title field, and a CompanyName-value pair associating a value of “ACME” to the company name field based on the email 652a illustrated in FIG. 6B. Because each electronic activity may include multiple strings having data that corresponds to a particular field, the node graph generation system can generate multiple activity-field value pairs from each electronic activity (e.g., multiple first name-value field pairs based on information from a sender field and a signature block).

Referring further to FIG. 6A, the node graph generation system 200 can maintain a plurality of node profiles 600. Each node profile 600 includes a plurality of node field-value pairs corresponding to attributes 610 and value data structures 615. For example, the node graph generation system 200 can maintain a first node field-value pair associating a first value 620 (e.g., Va) to field 610(1), a second node field-value pair associating a second value 620 (e.g., Vb) to the field 610(1), and so on for each value. As shown for the node profile illustrated in the table above, the node graph generation system 200 can generate a first field-value pair associating a value of John to the first name field, and a second field-value pair associating a value of Johnathan to the first name field.

The node graph generation system 200 can compare the activity field-value pairs of an electronic activity to be matched to respective node field-value pairs of one or more candidate node profiles with which to match the electronic activity. The node graph generation system 200 can compare one or more activity field-value pairs of the electronic activity to corresponding node field-value pairs of a candidate node profile to determine a match score between the electronic activity and the candidate node profile. The node graph generation system 200 can identify one or more node profiles with which to match the electronic activity based on the match score. Node profiles having a match score below a predetermined threshold can be determined not to be matched.

To compute the match score, the node graph generation system 200 can iterate through each activity field-value pair, identify the field of the activity field-value pair, and identify a corresponding field of a node field-value pair of the node profile. For example, the node graph generation system 200 can identify the field of the activity field-value pair to be first name, and based on the identification, select the field of the node field-value pairs that will be used for the comparison to be the first name field of the node field-value pairs. The node graph generation system 200 can retrieve the value from the activity field-value pair, retrieve a corresponding value that is associated to the identified field of the node field-value pair, and compare the values. For example, the node graph generation system 200 can select the first name field of a first activity field-value pair, identify a corresponding first name field of a first node field-value pair, retrieve the value of the first name from the first activity field-value pair, retrieve the corresponding value of the first name from the first node field-value pair, and compare the retrieved values. With reference to the electronic activity EA-003 and node profile NPID-12 of FIG. 6B, the node graph generation system can generate an activity field-value pair of FirstName:John, identify the field to be first name, identify the corresponding first name field of each node field-value pair of the node profile NPID-12, retrieve the first name John from the activity field-value pair, and retrieve the first name John from the node field-value pair (or the first name Johnathan from the second value that is assigned to the first name field of the node profile NPID-12). The node graph generation system 200 can compare the first name John of the activity field-value pair to the first name John of the node field-value pair, and calculate a match score based on the comparison. For example, the node graph generation system 200 can assign a match score of 100 percent based on the comparison of John and John. The node graph generation system 200 may assign a match score less than 100 percent based on the comparison of John and Johnathan. The node graph generation system 200 can calculate match scores for each comparison of the electronic activity and respective candidate node profiles.

The node graph generation system 200 can compare each match score between the electronic activity and the node profile to a match score threshold to determine whether the electronic activity is to be matched to the node profile. As such, the node graph generation system 200 can use the data extracted from the electronic activity to make decisions such as whether an electronic account associated with the electronic activity was a sender or a recipient of the electronic activity. The node graph generation system 200 can calculate an average (e.g., weighted average) of each match score determined for each comparison for the electronic activity, and compare the weighted average to the match score threshold to determine whether the electronic activity matches the node profile.

The node graph generation system 200 can apply various rules to determine how to calculate the weighted average. In some implementations, the node graph generation system 200 calculates the weighted average based on a measure of uniqueness of the field of the value used to calculate the match score. The node graph generation system 200 can apply different weights to different fields based on the rarity score of the field. The rarity score of the field can be determined by generating a count of each value of the field across all node profiles maintained by the node graph generation system. If a predetermined number or threshold of values have a frequency count that satisfies a predetermined threshold, the field can have a lower rarity score than another field in which none of the values have a frequency count that exceeds the predetermined threshold. For example, the field FirstName can have a low rarity score because there are a lot of common first names, such as John, Chris, Tom, Ben, Dave, Alex, etc. In contrast, the field Email can have a higher rarity score because email addresses are generally unique to individuals. In some implementations, the system may determine certain emails that may not be personal to an individual but rather belong to a group and the system can discount the influence those emails that belong to a group In some embodiments, info@example.com or help@example.com may be indicative of an email address that does not belong to an individual node profile. In this way, the node graph generation system 200 can assign a first rarity score to the first name field, a second rarity score greater than the first rarity score to the last name field, and a third rarity score greater than the second value to the phone number field. Responsive to the match score satisfying the match score threshold, the node graph generation system 200 can link the electronic activity to the node profile. For example, the node graph generation system 200 can maintain an association in a data structure, the association indicating that the electronic activity is linked to the node profile.

Linking an electronic activity to a node profile includes adding an entry to each value data structure of each value of each field of the node profile that is supported by the electronic activity. As an example, let's say the electronic activity is matched to a first node profile corresponding to John Smith corresponding to a sender of the electronic activity and a second node profile Abigail Xu corresponding to a recipient of the electronic activity. The system can identify each of the values of the fields of the sender's node profile that is supported by the electronic activity, such as the first name of the sender, the last name of the sender, the company of the sender, the email address of the sender and other fields that include values that can be supported by the signature of the sender included in the email. The system can then update the value data structure of each of those values by adding an entry identifying the electronic activity as a data point. As such, the electronic activity can serve as a data point for multiple values of multiple fields of a particular node profile. Similarly, the system can identify each of the values of the recipient's node profile that is supported by the electronic activity and can add entries in respective value data structures of values of fields of the recipient's node profile that are supported by the electronic activity. In this way, the electronic activity not only can update multiple value data structures of a single node profile but can also update the value data structures of multiple node profiles thereby multiplying the impact a single electronic activity can have towards the accuracy and state of the node profiles and the node graph in aggregate.

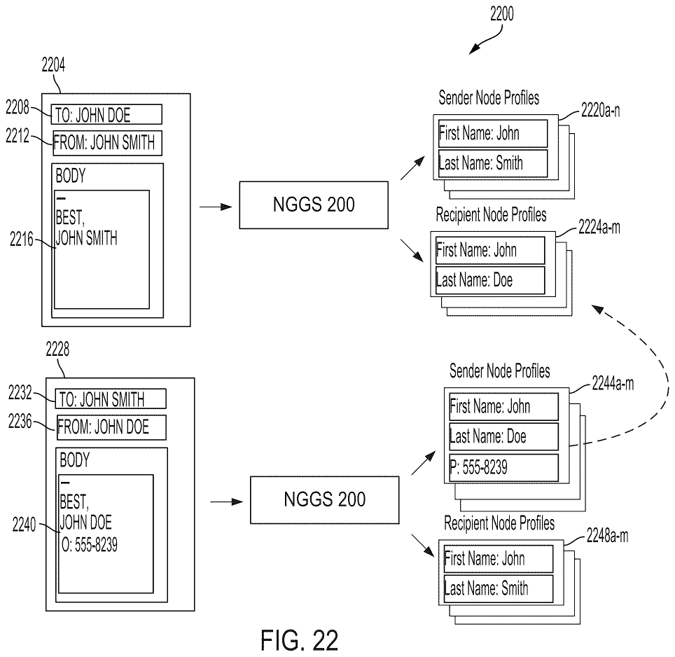

Referring now to FIG. 22, FIG. 22 illustrates a process flow 2200 in which the node graph generation system (NGGS) 200 can use relationship information among multiple electronic activities to more accurately identify the subset of node profiles to which to link the electronic activities. For example, the node graph generation system 200 can identify a sender of a first electronic activity, such as an email, and a recipient of the first electronic activity, and determine that a subsequent, second electronic activity sent by the recipient to the sender is a reply to the first electronic activity. Based on this relationship and based on information extracted from the second electronic activity used to identify a second subset of node profiles that the second electronic activity is to be linked to, such as node profiles that may potentially represent the recipient, the node graph generation system 200 can effectively increase a match score of linking the first electronic activity to node profiles of the second subset (which may not necessarily have been identified as node profiles of the recipient based only on information extracted from the first electronic activity). The node graph generation system 200 can execute the process 2200 in real-time by searching for and identifying such relationships responsive to ingesting each second electronic activity, and then updating the corresponding match scores of the first electronic activity based on the links made from the second electronic activity. The node graph generation system 200 can execute the process 2200 periodically and/or in near-real time, such as in a batch processing of electronic activities.

As illustrated in FIG. 22, the node graph generation system 200 can identify a first electronic activity 2204. The node graph generation system 200 can extract a first sender 2212 of the first electronic activity 2204, and determine the first sender to be John Smith (e.g., to have first name John and last name Smith). The node graph generation system 200 can extract a first recipient 2208 of the first electronic activity 2204, and determine the first recipient to be John Doe (e.g., to have first name John and last name Doe). For example, the node graph generation system 200 can extract, from the signature block 2216 of the first electronic activity 2204, FirstName and LastName strings, and determine the FirstName and LastName string to correspond to the sender 2212 based on the FirstName and LastName strings being extracted from the signature block 2216.

The node graph generation system 200 can execute various processes described herein to identify a subset of a plurality of node profiles that match the first electronic activity 2204. For example, the node graph generation system 200 can identify a subset of the plurality of node profiles that includes a first sender subset 2220a-2220n, and a first recipient subset 2224a-2224m. The node graph generation system 200 can respectively assign sender and recipient statuses to the first electronic activity 2204 when identifying the node profiles of the subsets 2220a-2220n, 2224a-2224m. The node graph generation system 200 can identify the first sender subset 2220a-2220n by determining that match scores of comparing the electronic activity 2204 to the first sender subset 2220a-2220n satisfy the match score threshold, and determining that match scores of comparing the first electronic activity 2204 to the first recipient subset 2224a-2224m satisfy the match score threshold. There may be node profiles for which the comparison results in match scores that do not satisfy the match score threshold, such as if data extracted from the first electronic activity 2204 does not match data of such node profiles, even if such node profiles should match the first electronic activity 22b04.

The node graph generation system 200 can identify a second electronic activity 2228. The node graph generation system 200 can extract a second recipient 2232 of the second electronic activity 2228, and determine the second recipient 2232 to be John Smith. The node graph generation system 200 can extract a second sender 2236 of the second electronic activity 2228, and determine the second sender 2236 to be John Doe. The node graph generation system 200 can extract, from the signature block 2240, first name, last name, and office (or cell) phone number information. The node graph generation system 200 can process the second electronic activity 2228 to identify a subset of node profiles that match the second electronic activity 2228, including a second sender subset 2244a-m and a second recipient subset 2248a-m.

The node graph generation system 200 can determine that the second electronic activity 2228 is a reply to or a forward of the first electronic activity 2204. For example, the node graph generation system 200 can process metadata of the second electronic activity 2228 to identify a status indicator indicating that the second electronic activity 2228 is a reply to or a forward of the first electronic activity 2204. The node graph generation system 200 can parse a subject line of the second electronic activity 2228 to determine that the second electronic activity 2228 is a reply to or a forward of the first electronic activity 2204, such as if the subject line of the second electronic activity 2228 includes a string of the subject line of the first electronic activity 2204 that has been appended to the characters “RE:” or “FW” (in various type cases of those characters).

Responsive to determining that the second electronic activity 2228 is a reply to the first electronic activity 2204, the node graph generation system 200 can determine at least one of (i) the recipient of the second electronic activity 2228 is the sender of the first electronic activity 2204 or (ii) the sender of the second electronic activity 2228 is the recipient of the first electronic activity 2204. Based on these determinations, the node graph generation system 200 can update the first recipient subset 2224a-m to include at least one node profile of the second sender subset 2244a-m that did not satisfy the match score threshold when the node graph generation system 200 initially identified the first recipient subset 2224a-m. For example, when identifying the second sender subset 2244a-m, the node graph generation system 200 may have identified at least one node profile that satisfied the match score threshold because of the phone number extracted from the signature block 2240. The node graph generation system 200 can update the first sender subset 2220a-n to include at least one node profile of the second recipient subset 2248a-m. By using the send and reply relationships of electronic activities 2204 and 2228, the node graph generation system 200 can more precisely identify the subsets 2220a-n, 2224a-m to which to link the first electronic activity 2204, and more precisely identify the subsets 2244a-m and 2248a-m to which to link the second electronic activity 2228.

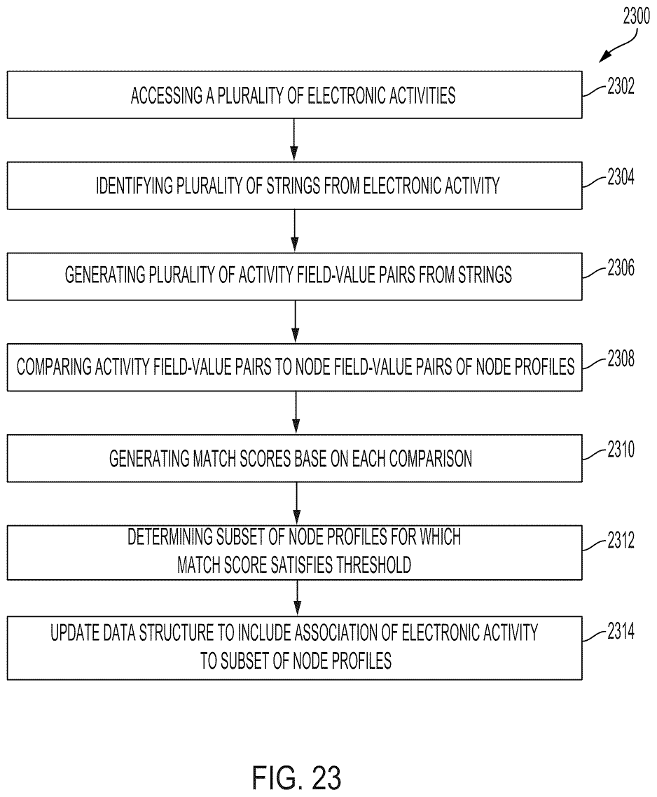

Referring now to FIG. 23, FIG. 23 illustrates a method 2300 of linking electronic activities to node profiles. The method 2300 can include accessing a plurality of electronic activities (BLOCK 2302). With reference to FIGS. 16-18, among others, the data processing system 9300 can access a plurality of electronic activities. The electronic activities can be transmitted to the data processing system 9300 from data source providers. The data processing system 9300 can retrieve the electronic activities from the data source providers. For example, the data source provider can include or be an email server. The data processing system 9300 can have the authority to access the emails stored on the email server through an API or an HTTP method (e.g., a GET method). The plurality of electronic activities can be received via electronic accounts associated with a plurality of data source providers.

The data processing system 9300 maintains a plurality of node profiles. Each node profile can include information such as first name, last name, company, and job title, each of which are represented by fields having one or more values, each value having a confidence score assigned to the value. The data processing system 9300 is configured to update the plurality of node profiles using the plurality of electronic activities.

The method 2300 can include identifying a plurality of strings from data included in an electronic activity of the plurality of electronic activities, such as to link the electronic activity to one or more node profiles (BLOCK 2304). For example, the data processing system 9300 can execute the electronic activity parser 210 to identify the plurality of strings. The plurality of strings can correspond to fields including a first name field, a last name field, an email address field, a phone number field, a title field, and a company field.

The method 2300 can include generating a plurality of activity field-value pairs from the plurality of strings (BLOCK 2306). For example, the data processing system 9300 can use an electronic activity parsing policy to generate the plurality of activity field-value pairs. Each activity field-value pair can include a data structure that associates a value extracted from a string of the plurality of strings to a particular field represented by the electronic activity. For example, the data processing system 9300 can identify a value of a first name from the electronic activity (e.g., “John”), and associate the identified value to a first name field, to generate an activity field-value pair such as First Name:John. The data processing system 9300 can use the electronic activity parser 210 to execute the electronic activity parsing policy, such as to identify strings from metadata as well as non-metadata of the electronic activity. For example, the data processing system 9300 can identify a first string from a portion of the electronic activity, and determine a confidence score that the first string is a first name by at least one of (i) comparing the first string to a plurality of values of a first name field of the plurality of node profiles or (ii) a portion of the electronic activity from which the first string was identified.

The method 2300 can include comparing the plurality of activity field-value pairs to respective node field-value pairs of one or more node profiles (BLOCK 2308). For example, the data processing system 9300 can compare each activity field-value pair to respective field-value pairs of each of the one or more node profiles to identify a subset of activity field-value pairs that match respective node field-value pairs of the one or more node profiles. The data processing system 9300 can identify a field of the activity field-value pair, retrieve the value associated to the identified field, identify a corresponding field of the node field-value pairs, and compare the value retrieved from the activity field-value pair to each value associated to the corresponding field each node field-value pair. The data processing system 9300 can identify a string type of the string corresponds to a field type of the activity field or the node field in order to retrieve the values that are compared.

The method 2300 can include generating a match score of the node profile indicating a likelihood that the electronic activity is transmitted or received by an account corresponding to the node profile based on the comparison (BLOCK 2310). The data processing system 9300 can generate the match scores using the comparisons of respective values of activity field-value pairs and node field-value pairs. For example, the data processing system 9300 can compare characters of the value (e.g., of the string from which the value is extracted) to values of the corresponding field of the activity-field value pairs. In some implementations, the data processing system 9300 determines a weighted average of a plurality of match scores for a plurality of values of the electronic activity (e.g., each value of each activity field-value pair). The data processing system 9300 can determine the weighted average by assigning a uniqueness score as a weight to each value used to determine the weighted average. For example, the data processing system 9300 can assign a uniqueness score based on the field to which the value is associated (e.g., a value of a first name field has a lesser uniqueness score than a value of a last name field, which has a lesser uniqueness score than a value of a phone number field). The data processing system 9300 can assign a uniqueness score based on a rarity of the value (e.g., some first names may be more rare than other first names); the data processing system 9300 can assign the uniqueness score of each value based on how many node profiles include the same value for the given field relative to the total number of node profiles.

The method 2300 can include determining a subset of the plurality of node profiles to which to link the electronic activity, responsive to determining that the match score of each node profile of the subset of the plurality of node profiles satisfies a threshold (BLOCK 2312). For example, the data processing system 9300 can compare each match score of each electronic activity (which may be a weighted average) to the threshold, and select the subset of the plurality of node profiles for which the comparison satisfies the threshold.

The method 2300 can include updating a data structure to include an association between the electronic activity and each node profile of the subset of the plurality of node profiles (BLOCK 2314). For example, the data processing system 9300 can generate a data structure that includes a link indicating a connection between the electronic activity and the node profiles of the subset of the plurality of node profiles. In some implementations, the data processing system 9300 adds entries to the node profiles of the subset of the plurality of node profiles to identify the electronic activity responsive to determining the electronic to match the node profiles. For example, the data processing system 9300 can add an entry to a value data structure of the value of the field of the node profile that is used to match the electronic activity to the node profile. For example, the data processing system 9300 can determine that the string used to match the electronic activity to the node profile is a string of a first name, such as the string having the value “John,” and in response can add an entry to a value data structure that includes the value John assigned to the first name field of the node profile to identify the electronic activity. The data processing system 9300 can determine that a second string used to match the electronic activity is a string of a second field type, such as a last name field type corresponding to the string having the value “Smith,” and in response can add a second entry to a value data structure that includes the value Smith assigned to the last name field of the node profile to identify the electronic activity. In some implementations, the node profile may not have an existing value data structure corresponding to each value retrieved from the electronic activity. For example, the data processing system 9300 can determine that the node profile has a value data structure that matches the value John for the first name field, but does not have a value data structure that matches the value Smith (retrieved from the same electronic activity as the value John) for the last name field, and the data processing system 9300 can generate a value data structure that includes the value Smith for the last name field.

In some implementations, the data processing system 9300 determines a contribution score of the entry that is added to identify the electronic activity. The data processing system 9300 can determine the contribution score based on a trust score of a source of the electronic activity. The contribution score can be indicative of the data point's contribution towards the confidence score of the value. The contribution score of a data point can decay over time as the data point becomes staler. For example, the contribution score can be based on a time at which the data point (e.g., the value) was generated or last updated. The data processing system 9300 can use the contribution score to determine a confidence score of the value. The data processing system 9300 can use each contribution score associated with each entry that indicates the value to calculate the confidence score of the value. For example, for the value “John” of a first name field, the data processing system 9300 can determine a weighted average of contribution scores of each electronic activity from which the value “John” is identified to determine the confidence score. By linking electronic activities to node profiles, and using contribution scores to determine the confidence score of each value, the data processing system 9300 can use the electronic activities as data points that support the value associated with the field, such as to enable an objective an accurate indication of the value that should correspond to the electronic account that the node profile represents.

In some implementations, the method 2300 includes selecting a first node profile of the subset of the plurality of node profiles based on the match score of the first node profile, and linking the electronic activity with the first node profile. For example, the data processing system 9300 can rank each node profile of the subset of the plurality of node profiles based on each respective match score, and select the first node profile as the node profile having a greatest match score in order to link the node profile having the greatest match score to the electronic activity.

### 18. Linking Record Objects to Node Profiles

The present solution can enable real-time or near real-time linking of record objects to node profiles, with increased accuracy. In some systems that maintain data regarding entities, such as individuals or enterprises, including systems of record, the data may be self-reported, such as in response to specific queries to provide data for fields such as first name, last name, title, or email. As such, this data may be inaccurate. For example, when the data was provided, the data may have been inaccurate due to the data being self-reported. At a particular instant in time after the data was provided, due to changes to the data that may have occurred subsequent to when the data was provided and before the data has been updated, the data even if it was previously correct at the time the data was provided, may also eventually become obsolete, stale or inaccurate.

The present solution described herein can match record objects of systems of record to node profiles maintained by a node graph generation system, that can use the data included in the record objects to update node profiles and the values of fields of these node profiles unobtrusively and without requiring any direct human input. As such, the present disclosure describes solutions for maintaining node profiles that remain accurate as the node profiles do not rely directly on self-reported information submitted by a user to update the node profile and because the node profiles are automatically updated as record objects are ingested and processed by the system without requiring any human activity. In this way, the present solution can enable dynamic updates to node profiles and a node graph including such node profiles, rather than manual/self-reported updates. Even if the underlying record objects include self-reported information, the present solution can maintain contribution scores of each data source of record objects, update the contribution scores based on verification of the data extracted from the data source, and determine confidence scores of each value of a field of the node profile based on the contribution scores of the record objects that support that value.

By linking record objects to node profiles, the present solution can increase the accuracy and validity of the node profiles, such as by increasing a likelihood that each node profile represents the true state of the world. For example, when node profiles are used to generate a node graph indicative of a hierarchy or other relationships amongst node profiles, the present solution can more accurately represent values of fields such as job titles that are used to generate the node graph. The present solution can more accurately rank each value of each field (each value representing a potential true state of the world) by dynamically updating the confidence score corresponding to each value responsive to extracting data from record objects, so that the present solution outputs an evidence-based estimation of which value is the true value with improved accuracy. As an example, a node profile can include a first email address corresponding to a first job and a second email corresponding to a subsequent job. Each of the two email addresses are at respective points in time, accurate and valid. As the person switches jobs, the first email address is no longer valid but the confidence score associated with the email address can in some embodiments, remain high indicating that the first email address belongs to the node profile. Similarly, the second email address also belongs to the node profile and therefore also has a high confidence score. After the system determines that the second email address is active and functioning, the system can assign a higher confidence score to the second email address relative to the first email address since the contribution scores provided by recent data points (for example, recent record objects identifying the second email address) can contribute towards the higher confidence score. The present solution can thus respond to changes in the true state of the world represented by the node profile using the second email, rather than relying on self-reported information which may be inaccurate and/or delayed.

Referring further to FIG. 2, among others, the node graph generation system 200 can ingest record objects to generate or update node profiles that are maintained by the node graph generation system 200 using data from the record objects. For example, as illustrated in FIG. 10, the node graph generation system 200 can process record objects or data records of a system of record, such as a customer relationship management (CRM) system. The node graph generation system 200 can process record objects of systems of records such as Applicant Tracking Systems (ATS), such as Lever, located in San Francisco, CA or Talend by Talend Inc., located in Redwood City, CA, enterprise resource planning (ERP) systems, customer success systems, such as Gainsight located in Redwood City, CA, Document Management Systems, among others. As illustrated in FIG. 10, the record objects can include a lead record object 1000, an account record object 1002, an opportunity record object 1004, or a contact record object 1006.

The node graph generation system 200 can process the record objects to identify a plurality of object fields of the record objects. For example, each record object can have one or more object field-value pairs. The node graph generation system 200 can process the record object to identify values from structured data fields of the record objects. In some embodiments, the node graph generation system 200 can execute a record object parsing policy to identify values from unstructured data of the record objects, such as by executing functions of the record data extractor 230. The node graph generation system 200 can generate a plurality of object field-value pairs that associate the identified values to the corresponding fields. Because each record object may include multiple strings having data that corresponds to a particular field, the node graph generation system 200 can generate multiple object field-value pairs from each electronic activity (e.g., multiple first name-value field pairs based on multiple record entries of the record object).

Referring further to FIG. 6A, the node graph generation system 200 can maintain a plurality of node profiles 600. Each node profile 600 includes a plurality of node field-value pairs corresponding to attributes 610 and value data structures 615. For example, the node graph generation system 200 can maintain a first node field-value pair associating a first value 620 (e.g., Va) to field 610(1), a second node field-value pair associating a second value 620 (e.g., Vb) to the field 610(1), and so on for each value. As shown for the node profile illustrated in the table above, the node graph generation system 200 can generate a first field-value pair associating a value of John to the first name field, and a second field-value pair associating a value of Johnathan to the first name field.

The node graph generation system 200 can compare the object field-value pairs of a record object to be matched to respective node field-value pairs of one or more candidate node profiles with which to match the record object. The node graph generation system 200 can compare one or more object field-value pairs of the record object to corresponding node field-value pairs of a candidate node profile to determine a match score between the record object and the candidate node profile. The node graph generation system 200 can identify a node profile with which to match the record object based on the match score. Node profiles having a match score below a predetermined threshold can be determined not to be matched.

To compute the match score, the node graph generation system 200 can iterate through each object field-value pair, identify the field of the node field-value pair, and identify a corresponding field of a node field-value pair of the node profile. For example, the node graph generation system 200 can identify the field of the object field-value pair to be first name, and based on the identification, select the field of the node field-value pairs that will be used for the comparison to be the first name field of the node field-value pairs. The node graph generation system 200 can retrieve the value from the object field-value pair, retrieve a corresponding value that is associated to the identified field of the node field-value pair, and compare the values. For example, the node graph generation system 200 can select the first name field of a first object field-value pair, identify a corresponding first name field of a first node field-value pair, retrieve the value of the first name from the first object field-value pair, retrieve the corresponding value of the first name from the first node field-value pair, and compare the retrieved values.

With reference to the account record object 1002 of FIG. 10 and node profile NPID-12 of FIG. 6B, the node graph generation system can generate an object field-value pair of Field 1.XYZ (e.g., “John”), identify the field to be first name, identify the corresponding first name field of each node field-value pair of the node profile NPID-12, retrieve the first name John from the object field-value pair, and retrieve the first name John from the node field-value pair (or the first name Johnathan from the second value that is assigned to the first name field of the node profile NPID-12). The node graph generation system 200 can compare the first name John of the object field-value pair to the first name John of the node field-value pair, and calculate a match score based on the comparison. The node graph generation system can then match object field value pairs of the record object with remaining field-value pairs of the node profile and based on the comparison of these object field value pairs and field-value pairs, determine a match score between the record object and the node profile based on a number of pairs that matched. In some embodiments, the match score can be based on which node field-value pairs matched. For instance, node field-value pairs that are more unique to node profiles in the node graph generation system can contribute more to the match score than node field-value pairs that are less unique. For example, the field-value pair for a very large company, such as Google may not be as unique as a cell phone number of a particular person. Moreover, node field-value pairs that have values with a higher confidence score can contribute more to the match score than field-value pairs that have values with a lower confidence score to improve the accuracy of linking or matching record objects to node profiles. The node graph generation system 200 can calculate match scores for each comparison of the record object and respective candidate node profiles.

The node graph generation system 200 can compare each match score between the record object and the node profile to a match score threshold to determine whether the record object is to be matched to the node profile. The node graph generation system 200 can calculate an average (e.g., weighted average) of each match score determined for each comparison for the record object, and compare the weighted average to the match score threshold to determine whether the record object matches the node profile.

The node graph generation system 200 can apply various rules to determine how to calculate the weighted average. In some implementations, the node graph generation system 200 calculates the weighted average based on a measure of uniqueness of the field of the value used to calculate the match score. The node graph generation system 200 can apply different weights to different fields based on the rarity score of the field. The rarity score of the field can be determined by generating a count of each value of the field across all node profiles maintained by the node graph generation system. If a predetermined number or threshold of values have a frequency count that satisfies a predetermined threshold, the field can have a lower rarity score than another field in which none of the values have a frequency count that exceeds the predetermined threshold. For example, the field FirstName can have a low rarity score because there are a lot of common first names, such as John, Chris, Tom, Ben, Dave, Alex, etc. In contrast, the field Email can have a higher rarity score because email addresses are generally unique to individuals. In some implementations, the system may determine certain emails that may not be personal to an individual but rather belong to a group and the system can discount the influence those emails that belong to a group In some embodiments, info@example.com or help@example.com may be indicative of an email address that does not belong to an individual node profile. In this way, the node graph generation system 200 can assign a first rarity score to the first name field, a second rarity score greater than the first rarity score to the last name field, and a third rarity score greater than the second value to the phone number field.

In some implementations, the node graph generation system 200 calculates the weighted average based on a measure of uniqueness of the value in addition to or in contrast to the rarity score of the field. For example, certain names may be more unique (e.g., rare) than other names. The node graph generation system 200 can maintain a uniqueness data structure mapping each value of each field of each of the plurality of node profiles to a corresponding uniqueness or frequency count, and retrieve the uniqueness using the value. The node graph generation system 200 can generate the uniqueness data structure using the plurality of node profiles, and update the uniqueness data structure responsive to receiving node profile data. For example, the node graph generation system 200 can count a number of each unique value of the node profiles, and calculate the uniqueness for each unique value based on the count. The node graph generation system 200 can thus rely more heavily on data extracted from the record object that has a higher likelihood of specifically corresponding to the node profile (e.g., rather than matching the record object from which the first name value of John was extracted to every node profile having the first name John).

Responsive to the match score satisfying the match score threshold, the node graph generation system 200 can link the electronic activity to the node profile. For example, the node graph generation system 200 can maintain an association in a data structure. The association can indicate that the electronic activity is linked to the node profile. The node graph generation system 200 can update a confidence score of each value of the node profile that matches corresponding value(s) extracted from the electronic activity.

Referring further to FIG. 10, the node graph generation system 200 can use relationship information amongst record objects to more precisely determine the subset of node profiles that match the record objects. For example, the node graph generation system 200 can identify that two record objects are linked based on data such as opportunity contact role (OCR) objects, a conversion of a lead record object 100 into a contact record object 1006, an account record object 1002, and an opportunity record object 1004, or other links between record objects, including explicit or implicit linking of record objects. Responsive to identifying a link between two record objects, the node graph generation system 200 can use the node profiles that are determined to match one of the record objects to update the matches to the other record object. For example, responsive to determining that the lead record object 1000 is linked to the account record object 1002, the node graph generation system 200 can increase a match score between the lead record object and node profiles that are matched to the account record object 1002. This can be useful in the event where a few node profiles have a high match score with a first node profile but there are many node profiles that are candidate matches to a second record object linked to the first record object. In such an event, the system can identify the node profiles linked to the first record object to identify the same node profiles from the many node profiles that are candidate matches to the second record object.

For example, the node graph generation system 200 can identify the lead record object 1000, and determine a first subset of node profiles that match the lead record object as described herein, such as by comparing object field-value pairs of the lead record object to node field-value pairs of the node profiles of the first subset and evaluating match scores of the comparison relating to a match score threshold. The node graph generation system 200 can determine that the lead record object 1000 is linked to the account record object 1002 from information included in the record objects or the system of record. The node graph generation system 200 can determine a second subset of node profiles that match the account record object 1002 as described herein, such as by comparing object field-value pairs of the account record object 1002 to node field-value pairs of the node profiles and evaluating match scores of the comparison relative to a match score threshold. In some implementations, responsive to determining that the account record object 1002 is linked to the lead record object 1000, the node graph generation system 200 can add the node profiles of the second subset to the node profiles of the first subset. In some implementations, responsive to determining that the account record object 1002 is linked to the lead record object 1000, the node graph generation system 200 increases a match score of the comparison of the node field-value pairs of the node profiles of the second subset to the object field-value pairs of the lead record object 1000. As such, even if certain record objects have incomplete information that may result in inaccurately low match scores, by using the linking between record objects, the node graph generation system 200 can more accurately identify the node profiles that match the record objects (and thus the node profiles to which to link the record objects). This improves the accuracy of the matches made between record objects and node profiles and further improves the accuracy of the values of the fields of the node profile, thereby improving the accuracy of the node graph and the insights and analytics derived from the node profiles and the node graph.

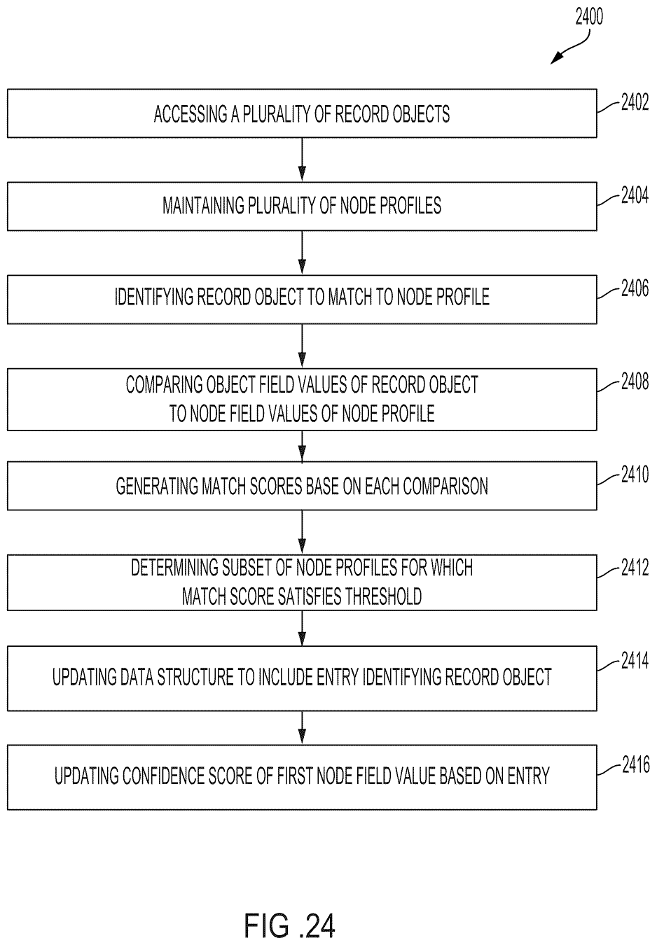

Referring now to FIG. 24, FIG. 24 illustrates a method 2400 of linking record objects to node profiles. The method 2400 can include accessing a plurality of record objects of one or more systems of record (BLOCK 2402). Each record object corresponds to a record object type (e.g., lead record object, account record object, among others) and includes one or more object field-value pairs associating an object field value to a corresponding field of the record object. The systems of record correspond to one or more data source providers. The data processing system 9300 can retrieve the record objects from the systems of record.

The method 2400 can include maintaining a plurality of node profiles corresponding to a plurality of unique entities (BLOCK 2404). Each node profile includes one or more field-value pairs associating a node field value to a corresponding field of the node profile. For example, the data processing system 9300 can maintain a plurality of node profiles that can include information such as first name, last name, company, and job title, each of which are represented by fields having one or more values, each value having a confidence score assigned to the value. The data processing system 9300 is configured to update the plurality of node profiles using the plurality of record objects.

The method 2400 can include identifying a record object to match to at least one node profile of the plurality of node profiles (BLOCK 2406). For example, the data processing system 9300 can parse the system of record of the record object to identify the record object periodically, or responsive to detecting or receiving an indication of an update to the system of record. The data processing system 9300 can identify from each record object a plurality of object field-value pairs that associate a value of an object field to the object field. For example, the data processing system 9300 can identify a first name field of the record object, extract the first name from the first name field, and generate the object field-value pair to associate the first name to the first name field.

The method 2400 can include comparing the object field values of the one or more object field-value pairs of the record object to corresponding node field values of the corresponding fields of the node profile (BLOCK 2408). For example, the data processing system 9300 can compare each object field-value pair to respective field-value pairs of each of the one or more node profiles to identify a subset of object field-value pairs that match respective node field-value pairs of the one or more node profiles. The data processing system 9300 can identify a field of the object field-value pair, retrieve the value associated to the identified field, identify a corresponding field of the node field-value pairs, and compare the value retrieved from the object field-value pair to each value associated to the corresponding field each node field-value pair.

The method 2400 can include generating a match score based on the comparison that indicates a likelihood that the record object corresponds to the node profile (BLOCK 2410). The data processing system 9300 can generate the match scores using the comparisons of respective values of object field-value pairs and node field-value pairs. For example, the data processing system 9300 can compare characters of the value (e.g., of the string from which the value is extracted) to values of the corresponding field of the object field-value pairs. In some implementations, the data processing system 9300 determines a weighted average of a plurality of match scores for a plurality of values of the record object (e.g., each value of each object field-value pair). The data processing system 9300 can determine the weighted average by assigning a uniqueness score as a weight to each value used to determine the weighted average. For example, the data processing system 9300 can assign a uniqueness score based on the field to which the value is associated (e.g., a value of a first name field has a lesser uniqueness score than a value of a last name field, which has a lesser uniqueness score than a value of a phone number field). The data processing system 9300 can assign a uniqueness score based on a rarity of the value (e.g., some first names may be more rare than other first names); the data processing system 9300 can assign the uniqueness score of each value based on how many node profiles include the same value for the given field relative to the total number of node profiles.

The method 2400 can include determining a subset of the plurality of node profiles with which to link the record object responsive to determining that the match score of each node profile of the subset satisfies a threshold (BLOCK 2412). For example, the data processing system 9300 can compare each match score of each record object (which may be a weighted average) to the threshold, and select the subset of the plurality of node profiles for which the comparison satisfies the threshold.

The method 2400 can include updating a first value data structure of the first node field value by adding an entry identifying the record object (BLOCK 2414). For example, the data processing system 9300 can generate a data structure that includes a link indicating a connection between the record object and the node profiles of the subset of the plurality of node profiles. In some implementations, the data processing system 9300 adds entries to the node profiles of the subset of the plurality of node profiles to identify the record object responsive to determining the record object to match the node profiles. For example, the data processing system 9300 can add an entry to a value data structure of the value of the field of the node profile that is used to match the record object to the node profile. For example, the data processing system 9300 can determine that the data used to match the record object to the node profile is of a first name field, such as a string having the value “John,” and in response can add an entry identifying the record object to a value data structure that includes the value John assigned to the first name field of the node profile. The data processing system 9300 can determine that a second string used to match the record object is of a second field type, such as a last name field type corresponding to the string having the value “Smith,” and in response can add a second entry identifying the record object to a value data structure that includes the value Smith assigned to the last name field of the node profile. In some implementations, the node profile may not have an existing value data structure corresponding to each value retrieved from the record object. For example, the data processing system 9300 can determine that the node profile has a value data structure that matches the value John for the first name field, but does not have a value data structure that matches the value Smith (retrieved from the same record object as the value John) for the last name field, and the data processing system 9300 can generate a value data structure that includes the value Smith for the last name field.

The method 2400 can include updating a confidence score of the first node field value based on the entry identifying the record object (BLOCK 2416). For example, the data processing system 9300 can increase the confidence score responsive to adding the entry, as the entry will further support an expectation that the value is a true, accurate value for that field. The data processing system 9300 can update the confidence score based on a contribution score of the entry. The contribution score can indicate a trustworthiness of the source of the entry, and can be updated over time by periodically comparing values of record objects retrieved from particular data sources (e.g., systems of record) to known values (or values having high confidence). For example, the data processing system 9300 can generate the contribution score based on a trust score assigned to the system of record that is associated with the record object. In some implementations, the data processing system 9300 determines the confidence score for the first node field value based on the contribution scores of the entries used to provide the values to the first node field value. For example, the data processing system 9300 can determine the confidence score based on an average of the contribution scores. The contribution score can be based on a time at which the record object was last updated or modified relative to a time at which the contribution score is calculated, such as to decrease the contribution score based on a difference between the two times.

In some implementations, the record object includes multiple object field-value pairs. The data processing system 9300 can match a first object field value to a first node field value, and a second object field value to a second node field value. The data processing system 9300 can generate a first confidence score for the first node field value based on the entry that identifies the record object, and can generate a second confidence score for the second node field value based on the entry that identifies the record object.

In some implementations, the data processing system 9300 maintains a shadow record object corresponding to the record object. Responsive to matching the record object to the subset of node profiles, the data processing system 9300 can add values from the node profile(s) of the subset to the shadow record object, which can facilitate completing or updating the shadow record object. For example, the data processing system 9300 can retrieve one or more values from the node profile(s) of the subset and add the one or more values to one or more shadow object fields of the shadow record object. In some implementations, the data processing system 9300 provides a notification to a device to update a value of the object field of the record object based on the one or more values added to the one or more shadow object fields of the shadow record object. As such, the data processing system 9300 can increase a completeness of the record object by matching the record object to the subset of node profiles, and then using values from the subset of node profiles to complete the record object. In some implementations, the data processing system 9300 identifies the values from the subset of node profiles to add to the shadow record object and/or the record object responsive to the confidence scores of the values satisfying a confidence score threshold, which can increase the accuracy of the completion of the shadow record object and/or the record object.

In some implementations, the data processing system 9300 uses values of the subset of node profiles to update the record object responsive to matching the record object to the subset of node profiles, which can increase the accuracy of the record object, and thus enable features such as more accurate determination of stages associated with the record object. For example, the data processing system 9300 can determine that a particular object field value of a field of the record object is different than a node field value of a corresponding field of the node profile. In response, the data processing system 9300 can retrieve the confidence score of the node field value, and compare the confidence score to a predetermined threshold. Responsive to the confidence score satisfying the predetermined threshold, such as being greater than the predetermined threshold, the data processing system 9300 can generate a request to update the object field value of the record object. The data processing system 9300 can generate the request to include the node field value that has the confidence score that satisfied the predetermined threshold and/or cause the system of record to update the object field value to be the node field value.

### 19. Generating Confidence Scores of Values of Fields Based on Data Points

The present disclosure relates to systems and methods for generating and updating confidence scores of values of one or more field of node profiles. By generating and updating confidence scores of values, a system can determine, at any point in time, a current state of the node profile while providing a level of confidence for each value. In existing systems that may maintain some form of a node profile, the node profile can include values that are static and only get updated responsive to a change made by a user. In the present disclosure, because the node profiles include value data structures that are continually updated by adding entries identifying new data points that support the value, the system is able to dynamically update the node profile without any user intervention, while at the same time, compute a confidence score of one or more values of the node profile. This allows a user querying the system to determine, at any given point in time, a state of the node profile, including the state of the node profile at any point in the past.

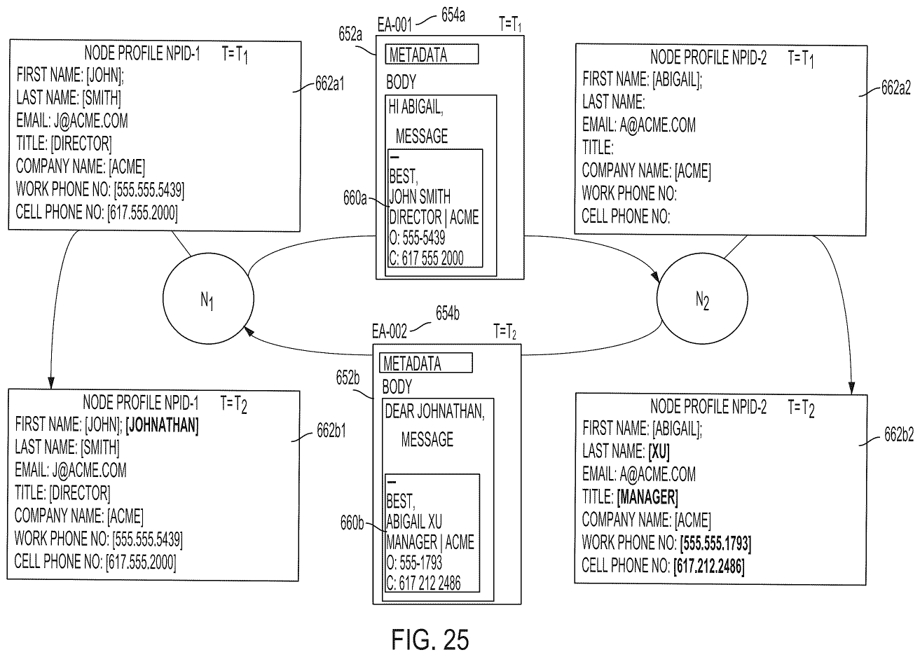

Moreover, the present disclosure can generate and update confidence scores of values of one or more fields of multiple node profiles. In some embodiments, the present disclosure can update node profiles, or more particularly, value data structures of node profiles as the system ingests and processes one or more electronic activities or record objects of systems of record. A single electronic activity or record object can serve as a data point for multiple value data structures of either a single node profile or multiple node profiles. In this way, a single electronic activity can update multiple node profiles simultaneously, accelerating the speed at which the system can generate and update node profiles and construct the node graph based on the node profiles. Based on the present disclosure, confidence scores of values can be generated and updated as more data points are processed, and as a result of dynamically and automatically updating confidence scores by assigning data points to values of fields of node profiles, the system can maintain and update a node graph of node profiles that is updated without human intervention and is more accurate than existing systems as it is dynamically updated, the source of data used to update the node profiles is not centralized and not reported by any one individual or user. Moreover, because electronic activities are constantly being generated, ingested and processed, the node profiles do not remain static. Referring now to FIG. 25, FIG. 25 illustrates a series of electronic activities between two nodes. As described herein, and also referring to FIGS. 6B and 8, a first node N1 and a second node N2 may exchange a series of electronic activities. FIG. 25 also shows a representation of two electronic activities and representations of two node profiles of two nodes at two different states according to embodiments of the present disclosure.

As shown in FIG. 25, a first electronic activity sent at a first time, T=T₁, and a second electronic activity sent at a second time, T=T₂, are shown. The first electronic activity 652a includes or is associated with a first electronic activity identifier 654a (“EA-001”). The second electronic activity 652b includes or is associated with a second electronic activity identifier 654b (“EA-002”). The system 200 can assign the first electronic activity identifier 654a to the first electronic activity 652a and second electronic activity identifier 654b to the second electronic activity 652b. In some embodiments, the system 200 can assign the first and second electronic activities' unique electronic activity identifiers to allow the system to uniquely identify each electronic activity processed by the system 200. Collectively, the first and second electronic activities can be referred to herein as electronic activities 652 or individually as electronic activity 652. Each electronic activity can include corresponding metadata, as described above, a body, and a respective signature 660a and 660b included in the body of the respective electronic activity 652.

The second electronic activity can be sent as a response to the first electronic activity. The system 200 can determine that the second electronic activity is a response to the first electronic activity using one or more response detection techniques based on signals included in the electronic activity including the metadata of the electronic activity, the subject line of the electronic activity, the participants of the electronic activity, and the body of the electronic activity. For instance, the system can determine that the second electronic activity has a timestamp after the first electronic activity. The system 200 can determine that the second electronic activity identifies the sender of the first electronic activity 652a as a recipient of the second electronic activity 652b. The system can determine that the second electronic activity includes a subject line that matches one or more words of the subject line of the first electronic activity. In some embodiments, the system can determine that the second electronic activity includes a subject line that includes the entire string of characters of the subject line of the first electronic activity and the string of characters is preceded by “RE:” or some other predetermined set of characters indicating that the second electronic activity is a reply. In some embodiments, the system can determine that the body of the second electronic activity includes the body of the first electronic activity. The system 200 can also determine that the second electronic activity is a response to the first electronic activity based on the participants included in both the electronic activities. Furthermore, in some embodiments, the system 200 can determine if the second electronic activity is a forward of the first electronic activity or a reply all of the first electronic activity.

FIG. 25 also includes two representations of two node profiles associated with the first node N1 and the second node N2 at two different times, T=T₁ and T=T₂. The node profile NPID-1 corresponds to a first node profile of the first node N1, who is the sender of the electronic activities 652a. The first representation 662a1 of the first node profile was updated after the first electronic activity 652a was ingested by the node graph generation system 200 but before the second electronic activity 652b was ingested by the system 200. The second representation 662b1 of the first node profile was updated after the first and second electronic activities 652a and 652b were ingested by the node graph generation system 200.

The node profile NPID-2 corresponds to a second node profile of one of the recipients of the electronic activity 652a and the sender of the second electronic activity 652b. The first representation 662a2 of the second node profile was updated after the first electronic activity 652a was ingested by the node graph generation system 200 but before the second electronic activity 652b was ingested by the system 200. The second representation 662b2 of the second node profile was updated after the first and second electronic activities 652a and 652b were ingested by the node graph generation system 200.

In some embodiments, as described herein, the node profile manager 220 of the system 200 can maintain, for each value of each field of each node profile, a value data structure that can be stored as a multidimensional array. The multidimensional array can include a list of entries identifying data points that identify electronic activities or system of records that contribute to the value of the field. Each data point can be associated with a source. For emails or other electronic activities, the source can be a mail server of a data source provider. For record objects, the source of the record object can be a system of record of the data source provider. Each source of a respective data point can have an associated trust score that can be used to determine how much weight to assign to the data point from that source. Each data point can also identify a time at which the data point was generated (for instance, in the case of a data point derived from an electronic activity such as an email, the time the data point was generated can be the time the electronic activity was sent or received). In the case of a data point being derived from a system of record, the time the data point was generated can be the time the data point can be entered into the system of record or the time the data point was last accessed, modified, confirmed, or otherwise validated in or by the system of record. The source of the data point and the time the data point was generated, last accessed, updated or modified, can be used to determine a contribution score of the data point, which can be used to determine the confidence score of the value. In some embodiments, the node profile manager 220 can generate, compute or assign a contribution score to each data point. The contribution score can be indicative of the data point's contribution towards the confidence score of the value. The contribution score of a data point can decay over time as the data point becomes staler. The contribution scores of each of the data points derived from electronic activities and systems of record can be used to compute the confidence score of the value of a field of the node profile.

Each of the representations 662 of the first and second node profiles can include fields and corresponding values. For example, in the first representation 662a1, the field “First Name” is associated with the value John. The first representation 662a1 of the first node profile also includes the field “Title” which is associated with the value “Director.” The values of the last name and cell phone number remain the same in both the representations 662a1 and 662b1 of the first node profile. In another example, in the first representation 662a2 of the second node profile, the field “First Name” is associated with the value Abigail. The first representation 662a2 of the second node profile does not include the field “Title” as that information may not have been available to the system 200. It should be appreciated that in the event the value was already associated with the field, the system 200 can update the value data structure of the value by adding an entry identifying the electronic activity. In this way, the electronic activity serves as a data point that supports the value and can increase the confidence score of the value, which can further improve the accuracy of the information included in the node profile.

In the representation 662b2 of the second node profile NPID-2, the second node profile was updated after the first and second electronic activities 652a and 652b were ingested. The field “Title” is now associated with the value “Manager.” The values of the “Work Phone No” and “Cell Phone No” fields have new values associated with them. In the representation 662b1 of the first node profile NPID-1, the first node profile was updated after the first and second electronic activities 652a and 652b were ingested. The field “First Name” is now associated with 2 different values, John and Johnathan. In the representative node profiles of NPID-1 and NPID-2, the same electronic activity can update different node profiles.

It should be appreciated that the value data structure of the value J@acme.com corresponding to the email field of the first node profile can be updated to include an entry identifying the second electronic activity 652b. It should further be appreciated that the system 200 is configured to updated the field-value pair of the first node profile corresponding to email: J@acme.com, even though J@acme.com is a value previously associated with the email field of the first node profile. The system can use the second electronic activity to update the node profile by not only adding new values, such as the name “Johnathan” but also by updating the value data structures of existing values of the first node profile to include entries identifying the second electronic activity 654b. By doing so, the system 200 can continuously maintain the accuracy of the data included in the node profiles and identify which values are still current and which values are now stale based on the last time a data point supported the particular value. As described herein, the system 200 can be configured to generate respective contribution scores to each entry included in the value data structure of a value and use the respective contribution scores of each entry of the value data structure to determine a confidence score of the value of the field of the node profile. The system can further be configured to dynamically update the contribution scores and the confidence score based on a current time as the contribution scores of data points can change with time. In some embodiments, the contribution scores of data points can decrease with time as the data point becomes older.

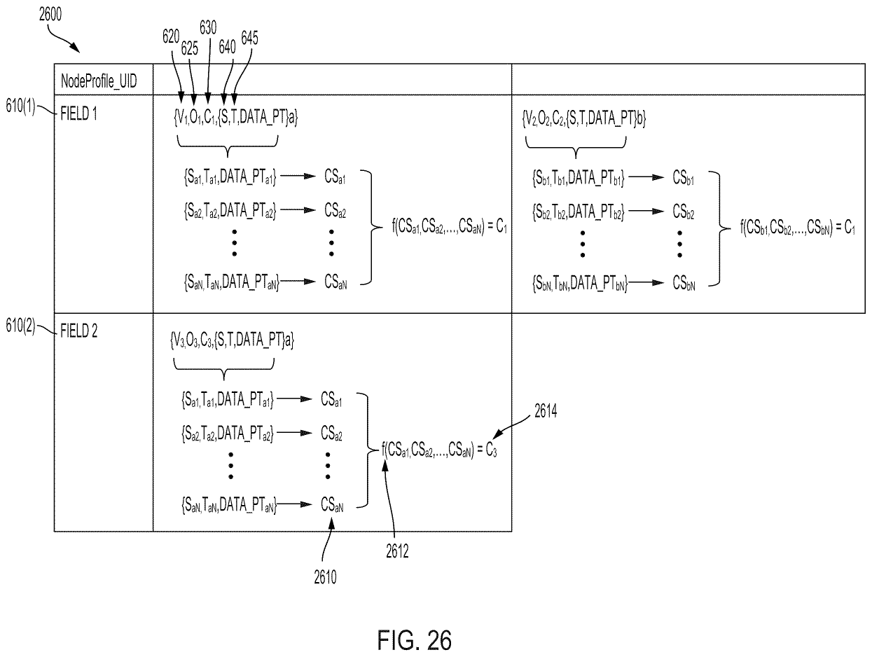

Referring now to FIG. 26, FIG. 26 illustrates a representation of a node profile 2600 of a node. As described herein, and also referring to FIG. 6A, the node profile can include one or more fields associated with one or more values. Each value can include a corresponding value data structure. The value data structure can include one or more entries. Each entry of the value data structure can identify a data point 2602 representing an electronic activity or a record object. In some embodiments, the node profile manager 220 can generate and assign a contribution score 2610 to each data point 2602 for the value to which the data point serves as evidence. The contribution score 2610 can be indicative of the data point's contribution towards the confidence score 2614 of the value. The contribution score 2610 of a data point 2602 can decay over time as the data point 2602 becomes staler. The contribution scores 2610 of each of the data points 2602 derived from electronic activities and systems of record can be used to compute the confidence score 2614 of the value of a field of the node profile.

As described herein, each of the values included in the node profile can be supported by one or more data points 2602. Data points 2602 can be pieces of information or evidence that can be used to support the existence of values of fields of node profiles. A data point 2602 can be an electronic activity, a record object of a system of record or other information that is accessible and processable by the system 200. In some embodiments, a data point 2602 can identify an electronic activity, a record object of a system of record, or other information that is accessible and processable by the system 200 that serves as a basis for supporting a value in a node profile. Each data point 2602 can be assigned its own unique identifier. Each data point 2602 can be associated with a source of the data point 2602 identifying an origin of the data point 2602. The source of the data point 2602 can be a mail server, a system of record, among others. Each of these data points 2602 can also include a timestamp. The timestamp of a data point 2602 can identify when the data point 2602 was either generated (in the case of an electronic activity such as an email) or the record object that serves as a source of the data point 2602 was last updated (in the case when the data point 2602 is extracted from a system of record). Each data point 2602 can further be associated with a trust score of the source of the data point 2602. The trust score of the source can be used to indicate how trustworthy or reliable the data point 2602 is. The data point 2602 can also be associated with a contribution score that can indicate how much the data point 2602 contributes towards a confidence score 2614 of the value associated with the data point 2602. The contribution score 2610 can be based on the trust score of the source (which is based in part on a health score of the source) and a time at which the data point 2602 was generated or last updated.

In some embodiments, a confidence score 2614 of the value can indicate a level of certainty that the value of the field is a current value of the field. The higher the confidence score 2614, the more certain the value of the field is the current value. The confidence score 2614 can be based on the contribution scores 2610 of individual data points 2602 associated with the value. The confidence score 2614 of the value can also depend on the corresponding contribution scores 2610 of other values of the field, or the contribution scores of data points 2602 associated with other values of the field.

Below is a reproduced portion of Table 1. The table illustrates various values for various fields and includes an array of data points that contribute to the respective value. As shown in the table, the same electronic activity can serve as different data points for different values. Further, the table illustrates a simplified form for the same of convenience and understanding.

| | | | | | | Trust | Contribution |
|---|---|---|---|---|---|---|---|
| | Data Point # | DP ID | TimeStamp | Activity ID | Source | Score | Score |
| Field: First Name | Value: John [Confidence score] = 0.8 | | | | | | |
| | Data Point 1: | DP ID101 | Feb. 1, 2016 4 pm ET | EA-003 | Email | 100 | 0.6 |
| | Data Point 2: | DP ID225 | Feb. 18, 2017 2 pm ET | SOR-012 | CRM | 70 | 0.4 |
| | Data Point 3: | DP ID343 | Mar. 1, 2018 1 pm ET | EA-017 | Email | 100 | 0.7 |
| | Data Point 4: | DP ID458 | Jul. 1, 2018 3 pm ET | EA-098 | Email | 100 | 0.8 |
| | Data Point 5: | DP ID576 | Sep. 12, 2015 3 pm ET | SOR-145 | Talend | 20 | 0.2 |
| Field: First Name | Value: Johnathan [Confidence score] = 0.78 | | | | | | |
| | Data Point 1: | DP ID101 | Feb. 1, 2016 4 pm ET | EA-003 | Email | 100 | 0.6 |
| | Data Point 2: | DP ID225 | Feb. 18, 2017 2 pm ET | SOR-012 | CRM | 70 | 0.4 |
| | Data Point 3: | DP ID343 | Mar. 1, 2018 1 pm ET | E4-017 | Email | 100 | 0.7 |
| | Data Point 4: | DP ID458 | Jul. 1, 2018 3 pm ET | EA-098 | Email | 100 | 0.8 |
| | Data Point 5: | DP ID576 | Sep. 12, 2015 3 pm ET | SOR-145 | Talend | 20 | 0.2 |
| Field: Title | Value: Director [Confidence score] = 0.5 | | | | | | |
| | Data Point 1: | DP ID101 | Feb. 1, 2016 4 pm ET | EA-003 | Email | 100 | 0.6 |
| | Data Point 2: | DP ID225 | Feb. 18, 2017 2 pm ET | SOR-012 | CRM | 70 | 0.4 |
| | Data Point 3: | DP ID243 | Mar. 1, 2017 1 pm ET | EA-117 | Email | 100 | 0.65 |
| | Data Point 4: | DP ID243 | Mar. 1, 2018 1 pm ET | SOR-087 | CRM | 5 | 0.05 |
| Field: Title | Value: CEO [Confidence score] = 0.9 | | | | | | |
| | Data Point 1: | DP ID343 | Mar. 1, 2018 1 pm ET | EA-017 | Email | 100 | 0.7 |
| | Data Point 2: | DP ID458 | Jul. 1, 2018 3 pm ET | EA-098 | Email | 100 | 0.8 |
| | Data Point 3: | DP ID225 | Mar. 18, 2018 2 pm ET | SOR-015 | CRM | 65 | 0.54 |

As a result of populating values of fields of node profiles using electronic activities, the node profile manager 220 can generate a node profile that is unobtrusively generated from electronic activities that traverse networks. In some embodiments, the node profile manager 220 can generate a node profile that is unobtrusively generated from electronic activities and systems of record.

As described herein, the present disclosure relates to methods and systems for assigning contribution scores to each data point (for example, electronic activity) that contributes to a value of a field such that the same electronic activity can assign different contribution scores to different values of a single node profile and of multiple node profiles. The contribution score can be based on a number of different electronic activities contributing to a given value of a field of a node profile, a recency of the electronic activity, among others. In some embodiments, a system of record of an enterprise accessible to the node graph generation system 200 can include data that can also contribute to a value of a field of a node profile. The contribution score can be based on a trust score or health score of the system of record. In some embodiments, the contribution score can be based on a number of different electronic activities or systems of record contributing to the value of the field of the node profile. In some embodiments, the contribution score can be based on a number of different electronic activities or systems of record contributing to other values of the field of the node profile. In some embodiments, the contribution score can be based on when the value was last updated or modified within the system of record, among others.

Referring back to Table 1, various factors can affect the contribution score of a given data point 2602. For example, a high trust score of a source of the data point can promote a higher contribution score. Data points corresponding to electronic activities generally have a higher contribution score while data points corresponding to systems of record with lower trust scores can have a lower contribution score. This may be because systems of record generally include data that is manually input by a user and remains static until it is modified. In contrast, the data in electronic activities, such as emails, are generated by multiple senders and include signatures that are updated by the creator of the signature i.e., the sender of the email. Although it is possible that an individual user may include incorrect information in their signature, they have more opportunities to correct such information and it can be confirmed or refuted based on other signals or electronic activities processed by the node graph generation system 200. Furthermore, the contribution score of a data point decreases as the data point gets older or the date associated with the last update of the data points gets older as can be seen by the contribution scores of the data points shown in the table above.

The system 200 can be configured to compute confidence scores using the contribution scores of individual data points identified by entries in the value data structure of the value for which the confidence score is being generated. The system 200 can compute confidence scores periodically. In some embodiments, the system 200 can update the confidence score of a value when additional data points are added. In some embodiments, the system 200 can compute the confidence score of a value based on a predetermined time schedule. The confidence score of a value can be a function of the contribution scores of various data points supporting the value (i.e. included in the value data structure of the value). The confidence score of the value can also decrease over time if no additional data points support the value. This is because the contribution scores of the data points that support the value will get older and since the contribution score of a data point is based on recency, the contribution scores will decrease resulting in a decrease in the confidence score. As such, to maintain a high confidence score of a value, newer entries need to be added to the value data structure. Via this mechanism of maintaining a dynamically updated value data structure that continually adds entries corresponding to data points that support the value, the node graph generation system 200 can continually compute and update a confidence score of a value based on the data points included in the corresponding value data structure.

By maintaining and periodically updating confidence scores of values, the system 200 can be configured to determine if electronic activities are correctly linked or matched to the right node profiles. For instance, if a given data point contributes to multiple field-value pairs of the node profile and a predetermined number of values of the field-value pairs have a confidence score below a threshold, the system can identify those data points that contribute to those values that have a confidence score below the threshold. The system can determine, for each of those data points, how many values of fields of the node profile does that data point provide support. The system can then identify those data points as candidate data points that were correctly linked or matched to the node profile. The system can then determine that the data point is improperly linked based on the number of values of the fields of the node profiles and the type of fields to which the data point provides support.

The system can then unmatch or delink the data point from the node profile by adjusting or recalculating the match score of the data point and the node profile. In this way, using the confidence scores of values, the system 200 can identify data points that were incorrectly linked to the node profile thereby further improving the accuracy of the node profile by removing data points that were previously incorrectly matched.

The systems described herein can also use confidence scores of values to determine a status of a node profile at a given point in time, for instance, 1:55 pm on Jun. 5, 2014. To do so, the node graph generation system 200 can discard all of the data points having a timestamp after 1:55 pm on Jun. 5, 2014 and only use data points before 1:55 pm on Jun. 5, 2014 that are included in the node profile. The node graph generation system 200 can then compute confidence scores of the field-value pairs of the node profile using the remaining or undiscarded data points to determine a state of the node profile on 1:55 pm on Jun. 5, 2014. The node graph generation system 200 can use the confidence score of each field-value pair to determine the state of the node profile at the given time. In this way, confidence state of a node profile can be determined for any point in time. As described herein, the node graph generation system 200 can make a request to determine a status of a node profile at any given point in time. For instance, the node graph generation system 200 can determine the state of a node profile for John Smith on Dec. 20, 2016. Similarly, the node graph generation system 200 can make a query for a particular value of a field of a node profile can be made for any point in time. Entries occurring after the particular time corresponding to the query can be filtered out so a value and its associated confidence score can be calculated using only those data points that have a timestamp before the particular time.

By determining confidence scores of field-value pairs of node profiles, the node graph generation system 200 can be configured to execute various types of requests based on the node profiles maintained by the node graph generation system 200. For instance, the system can be configured to determine a list of node profiles that have a title of Director. The system can be configured to determine a list of node profiles that have a title of Director on Jun. 1, 2015. The system can further be configured to determine a list of node profiles that work in San Francisco and have a title of CEO. Moreover, the system can be configured to determine only those node profiles that have a Company name of “ExampleCompany, Inc.” but with a confidence score for that field-value pair of above 90%. Using confidence scores as a threshold for selecting node profiles to be included in lists responsive to queries and adjusting the confidence score threshold to see changes in the lists can be a useful tool to identify node profiles with different levels of certainty.

In some embodiments, the present disclosure describes systems and methods of updating confidence scores of values of fields based on electronic activity includes associating the electronic activity to a first value of a first field, assigning a first contribution score to the electronic activity indicating a contribution level of the electronic activity to a confidence score of the first value, associating the same electronic activity to a second value of a second field, assigning a second contribution score to the same electronic activity indicating a contribution level of the electronic activity to a confidence score of the second value, and updating the confidence scores of the first value and the second value based on the first contribution score of the electronic activity for the first value and the second contribution score of the electronic activity for the second value.

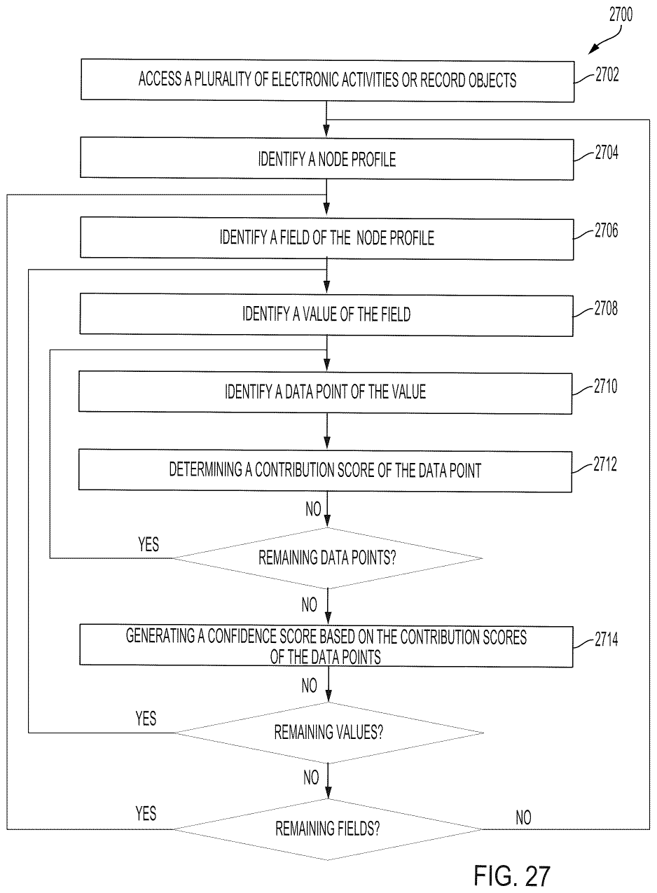

FIG. 27 illustrates a method 2700 to generate confidence scores of values of fields based on data points. The method 2700 can include accessing a plurality of electronic activities or record objects (BLOCK 2702). The method can identify, from a plurality of node profiles maintained by the data processing system, a first node profile that includes a plurality of fields, each having corresponding one or more values (BLOCK 2704). The method can identify, for a given node profile, a field of the plurality of fields (BLOCK 2706). The method can then identify, for a given field of the plurality of fields of the node profile, a given value of one or more values associated with the field (BLOCK 2708). The method can then identify, for the given value, a data point of one or more data points identified in the value data structure of the value (BLOCK 2710). The method can determine a contribution score of the data point (BLOCK 2712). The method can then determine, if the value data structure of the value includes any other data points that support the value. If additional data points exist in the value data structure, the method can determine the contribution score of the additional data points until the contribution score of each of the data points of the value data structure are determined. If no other data points exist, the method can determine a confidence score of the value based on the contribution scores of one or more of the data points supporting the value (BLOCK 2714). The method can optionally determine if the field of the node profile includes additional values for which a confidence score is to be determined. The method can then repeat the steps from blocks 2708-2714. The method can similarly determine the confidence scores of values of other fields by repeating Blocks 2706-2014. The method can similarly determine the confidence scores of values of other node profiles by repeating Blocks 2704-3014.

In further detail, the data processing system can access a plurality of electronic activities or record objects (BLOCK 2702). The data processing system can access the electronic activities can access a plurality of electronic activities via one or more servers hosting or storing the electronic activities. The servers can store electronic activities transmitted from or received by accounts corresponding to an enterprise. For instance, the servers can be mail servers, phone log servers, calendar servers or any other entity that can store emails, calendar events, phone logs, or other electronic activities of accounts associated with an enterprise, such as a company. The data processing system 9300 can be provided authorization to access the emails stored on one or more email servers through an API or an HTTP method (e.g., a GET method). Similarly, the data processing system can access record objects of one or more systems of record. Each system of record can be managed, owned, maintained or otherwise accessed by an enterprise. The enterprise can provide the data processing system access, permission or other information that enables the data processing system to access data included in the system of record. The data processing system can access the electronic activities and the record objects of one or more data source providers.

The data processing system 9300 accessing the plurality of electronic activities and/or the record objects of the system of record can further maintain a plurality of node profiles. The node profiles can be representations of nodes and includes fields that have values that are generated by data included in the plurality of electronic activities and/or record objects accessible by the system. The system can update the plurality of node profiles using at least one of the plurality of electronic activities or the plurality of record objects. Details regarding node profiles are described herein.

The method 2700 can include identifying a node profile from the plurality of node profiles maintained by the data processing system (BLOCK 2704). The data processing system can identify a node profile of the plurality of node profiles for which the system is to compute one or more confidence scores for one or more values of the node profile. The system can identify a particular node profile or can identify multiple node profiles for which the confidence scores of values of the node profile are to be determined. In some embodiments, the system can be configured to periodically compute confidence scores of node profiles. The system can identify a first node profile responsive to an update to the first node profile or responsive to linking an electronic activity or record object to the first node profile. In some embodiments, the system can identify a first node profile responsive to an update to the first node profile or responsive to adding an entry identifying an electronic activity or record object to a value data structure of a value of a field of the first node profile.

The method 2700 can include identifying a field of the node profile (BLOCK 2706). The system can identify a field of the node profile for which to compute the one or more confidence scores for the one or more values of the field. In some embodiments, the system can identify a first field of a plurality of fields of the identified node profile responsive to an update to the field of the node profile or responsive to adding an entry identifying an electronic activity or record object to a value data structure of a value of the field.

The method 2700 can include identifying a value of the field for which to determine a confidence score for which to compute the confidence score (BLOCK 2708). In some embodiments, the system can identify a value of one or more values associated with the field identified in BLOCK 2706. The system can identify the value responsive to an update to the value of the field or responsive to adding an entry identifying an electronic activity or record object to a value data structure of the identified value.

In some implementations, the value of the field of the node profile includes a first value of a first field of the node profile. The contribution score of the data point can be a first contribution score of a first data point. The confidence score can be a first confidence score of a first value. The data processing system 9300 can identify a second value data structure of a second field of the node profile. The second value data structure corresponds to a second value of the second field and further includes one or more second entries corresponding to respective one or more second data points that support the second value of the second value data structure. For example, and referring to Table 1, the value data structure corresponding to field-value pair First Name: Johnathan can include the value Johnathan and a first data point DP ID101 and a second data point DP ID225. The data processing system 9300 can determine, for at least one second data point of the one or more second data points of the second value of the second field of the node profile, a second contribution score of the second data point based on a time corresponding to when the second data point was generated or updated. For example, and referring to Table 1, a contribution score of 0.4 can be determined for DP ID225 based on the time corresponding to when the data was generated or updated (2/18/2017, 2PM ET). The data processing system 9300 can generate a second confidence score of the second value of the second field of the node profile based on the second contribution score of the at least one second data point. For example, and referring to Table 1, the confidence score of field-value pair First Name: Johnathan can be updated based on the DP ID225.

To determine a confidence score of the value identified in BLOCK 2708, the method 2700 can include identifying a data point that supports the existence of the value (BLOCK 2710). The data processing system can identify a data point of the value by identifying an entry of a value data structure of the value. The entry can identify the data point, a source of the data point, a trust score of a source of the data point and a timestamp associated with the data point. As described herein, for any given value of a field of a node profile, the value is associated with a value data structure that includes one or more entries. Each entry of the one or more entries can correspond to one or more data points that include a string that matches the value of the value data structure. For example, and referring to Table 1, for the field-value pair First Name: John, the entry corresponding to Data Point 1 can include a string in the electronic activity EA-003 that matches the value John. Each data point of the one or more data points can identify a respective electronic activity of the plurality of electronic activities or a respective record object of the plurality of record objects. For example, and referring to Table 1, for the field-value pair Field: Director, Data Point 1 (DP ID101) can be associated with electronic activity EA-003, Data Point 2 (DP ID2265) can be associated with system of record SOR-012, and Data Point 3 (DP ID243) can be associated with electronic activity EA-117. The data point identifies an electronic activity of the plurality of electronic activities or a record object of a system of record previously linked by the data processing system to the node profile associated with the value

The method 2700 can include determining a contribution score of the data point (BLOCK 2712). The data processing system can determine for at least one data point of the one or more data points included in a respective value data structure of the value (determined in BLOCK 2708) of the field (determined in BLOCK 2706) of the node profile (determined in BLOCK 2704), a contribution score of the data point based on a time corresponding to when the data point was most recently generated or updated. For example, and as illustrated in FIG. 26, the contribution score CSₐ₁ can be based on the time Tₐ₁ when the data point DATA_PTₐ₁ was generated or updated.

In some implementations, the data processing system 9300 can determine for the at least one data point of the one or more data points, the contribution score of the data point includes determining, for the at least one data point, a contribution score of the data point based on a trust score assigned to a source of the data point, the trust score determined based on a type of source of the data point. For example, and with reference to Table 1, the contribution score of DP ID101 corresponding to field-value pair First Name: John is 0.6 and is based on the trust score of 100. In some implementations in which the data point is a record object, the trust score assigned to the data point is based on a health of the system of record from which the record object was accessed. In some implementations, the health of the system of record from which the record object was accessed is determined based on comparing field values of object fields included in record objects of the system of record to node profile field values of fields of one or more node profiles having respective confidence scores above a predetermined threshold.

The method 2700 can include generating a confidence score based on the contribution scores of the data points (BLOCK 2714). The data processing system 9300 can generate a confidence score of the value of the field of the node profile based on the contribution score of the at least one data point. In some embodiments, the data processing system can generate the confidence score of the value based on the contribution score of each of the data points identified in entries of the value data structure of the value. For example, and as illustrated in FIG. 26, the confidence score C 1 is a function of the contribution scores CSₐ₁, CSₐ₂, . . . , CS_{aN}. The confidence score C₂ can be based on the contribution scores CS_{b1}, CS_{b2}, . . . , CS_{bN}. The confidence score C₃ can be based on the contribution scores CSₐ₁, CSₐ₂, . . . , CS_{aN}.

In some implementations, a data point identifies an electronic activity is an automatically generated bounce back electronic activity. Examples of bounce back electronic activity can include emails indicating that the destination email address is invalid or incorrect, the person is no longer with company, among others. In some implementations, the node profile includes a first field having a first value data structure identifying a first value. The first value can be assigned to the first field by linking a first electronic activity to the node profile

In some implementations the data processing system can receive a second electronic activity. The data processing system can determine that the second electronic activity includes or supports the value of the field of the node profile. For example, and with reference to Table 1, an electronic activity EA-098 can include a value John of the field First Name. The data processing system can match the electronic activity to the node profile and add an entry in one or more value data structures corresponding to field-value pairs of the node profile that are supported by the electronic activity. The system can determine which field-value pairs of the node profile are supported by the electronic activity as this information is used for matching electronic activities to node profiles. the system can then determine a contribution score of the second electronic activity for each field-value pair of the node profile that the second electronic activity supports. The data processing system can generate a second contribution score of the second electronic activity for the value of the field of the node profile as described herein. For example, and with reference to Table 1, a contribution score for DP ID458 associated with electronic activity EA-098 and field-value pair First Name: John can be generated. The data processing system can update the confidence score of the value based on the contribution score of the second electronic activity. The confidence score for field-value pair First Name: John can be updated based on the contribution score of DP ID458 that identifies the electronic activity EA-098.

In some implementations, the data processing system can identify the first electronic activity. The first electronic activity can be linked to the first node profile by identifying from data included in the first electronic activity, a plurality of strings. For example, and as illustrated in FIG. 25, data included in electronic activity 652a can be identified. The strings that can be identified from the data can include “John Smith”, “Director”, “ACME”, “555-5439”, “617.555.2000”, “j@acme”, “a@acme”, and “Abigail.” In some implementations, the electronic activity includes a signature block in the electronic activity and linking the electronic activity to the node profile includes the data processing system 9300 extracting a plurality of strings from the signature block of the electronic activity.

The data processing system can identify a plurality of candidate node profiles to which to link the electronic activity by comparing one or more strings of the plurality of strings to values of fields of respective candidate node profiles. For example, the data processing system can identify that node profile NPID-1 and node profile NPID-2 are candidate node profiles because they contain field-value pairs associated with the strings identified from the data included in electronic activity 652a. The data processing system can generate, for each candidate node profile, a match score indicating a likelihood that the electronic activity is transmitted or received by an account corresponding to the candidate node profile based on comparing the plurality of strings included in the electronic activity to values of fields included in the candidate node profile. The match score can be based on a number of fields of the node profile including a value that matches a value or string in the electronic activity. The match score can also be based on different weights applied to different fields. The weights may be based on the uniqueness of values of the field, as mentioned above. The data processing system can be configured to match the electronic activity to the node with the greatest match score. In some embodiments, the data processing system can match the electronic activity to each candidate node that has a match score that exceeds a predetermined threshold. Further, the data processing system can maintain a match score for each electronic activity to that particular node profile, or to each value of the node profile to which the electronic activity matched. By doing so, the data processing system can use the match score to determine how much weight to assign to that particular electronic activity. The data processing system 9300 can link the first electronic activity to the first node profile based on the match score of the first node profile. For example, the strings “John Smith”, “Director”, “ACME”, “555-5439”, “617.555.2000”, and “j@acme” can have a high match score to node profile NPID-1 and be linked to node profile NPID-1. The strings “a@acme” and “Abigail” can have a high match score to node profile NPID-2 and can be linked to node profile NPID-2.

In some implementations the data processing system 9300 can identify a record object of a system of record previously not matched to the value of the field of the node profile. The data processing system 9300 can determine that the record object includes the value of the field of the node profile. The data processing system can then add an entry identifying the record object to a value data structure of the value. The data processing system 9300 can generate a contribution score of the record object. The contribution score of the record object indicates a level of contribution of the record object to the value of the field. For instance, if the value is the name “John” for the field “First Name” of a particular node profile, the record object can be identified in an entry of the value data structure of the field-value pair “First Name: John” for the particular node profile if the record object includes a corresponding name-value pair that supports the field-value pair “First Name: John” for the particular node profile. To do so, the data processing system will first have to match the record object to the particular node profile and then add an entry to a value data structure of the field-value pair “First Name: John.” The data processing system can compute a new confidence score for the value based on the contribution score of the record object.

In some implementations the data processing system 9300 can receive a subsequent electronic activity. The data processing system 9300 can link the electronic activity to the node profile by including one or more entries identifying the electronic activity to one or more value data structures corresponding to one or more values of one or more fields. The data processing system can generate for each entry identifying the electronic activity, a contribution score of the electronic activity, the entry corresponding to a respective value data structure of a respective value of a respective field. The data processing system can generate respective confidence scores for the values based on the respective contribution scores of the electronic activity.

It should be appreciated that as described herein, electronic activities can include electronic activities that are transmitted or received via electronic accounts, or data derived from such electronic activities. The data may be derived from the electronic activities by parsing the electronic activities to extract information that can be used to generate tags from the electronic activities, identify one or more field-value pairs of node profiles, generate one or more activity field-value pairs that can correspond to one or more participants of the electronic activity, among others. The data can include tags, words, strings, field-value pairs, features or any other information that can be extracted or otherwise derived from an electronic activity. The electronic activities can be parsed by entities other than the data processing system 9300.

20. Detecting Events Based on Updates to Node Profiles from Electronic Activities

The present disclosure relates to systems and methods for detecting events based on updates to node profiles from electronic activities. The system can accurately and objectively determine event occurrences without relying on self-reported information. For example, the system can determine event occurrences (e.g., job movement/changes, promotions, marriage) based on information from electronic activities. The system can determine event occurrences by detecting bounce back electronic activity or by detecting a state change in node profiles. As described in the systems and methods herein, electronic activities can be parsed to update node profiles and improve the accuracy of field-value pairs of node profiles. In some instances, as new electronic activities are processed, the system can identify, in real-time, that an individual has been promoted, changed jobs, or changed names by detecting changes in one or more field-value pairs of a node profile corresponding to the individual. By way of the systems and methods described herein, the system can detect and determine events for individuals that communicate with one another, and as a result, maintain an accurate, dynamic, and objective node graph that does not rely on self-reported information. Furthermore, the systems and methods described herein can allow for the automatic inference of events and various information corresponding to the individuals. The present solution can enable more accurate and targeted electronic actions and improved detection of organizational hierarchies represented by node profiles.

In some embodiments, the data processing system 9300 can execute instructions that cause one or more processors to perform a method including accessing an electronic activity transmitted or received via an electronic account associated with a data source provider. The method may include generating a plurality of activity field-value pairs. The method may include maintaining a plurality of node profiles. The method may include identifying a first state of a first node profile. The method may include updating the first node profile. The method may include identifying a second state of the first node profile. The method may include detecting a state change of the first node profile. The method may include determining that the state change satisfies an event condition. The method may include storing an association between the first node profile and an event type corresponding to the event condition.

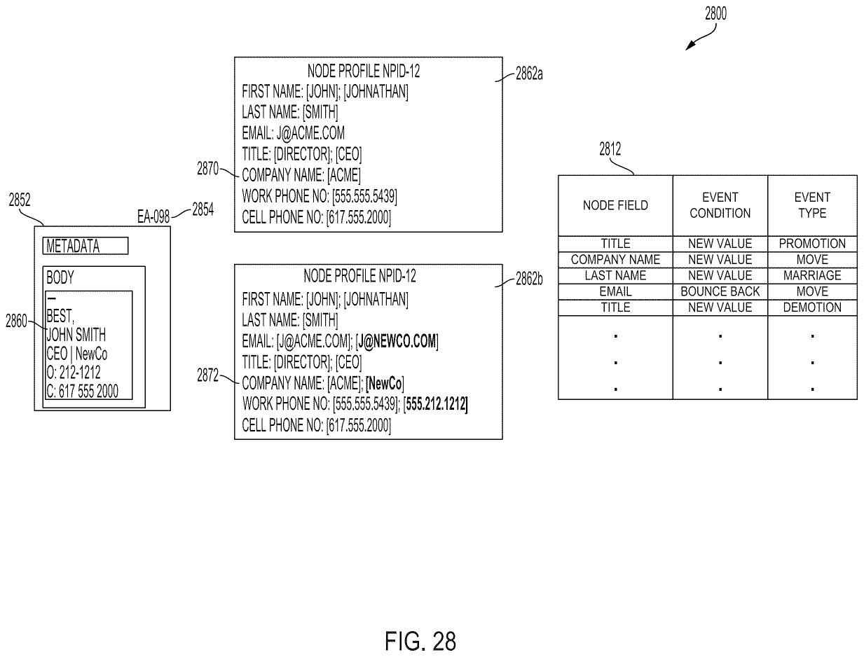

FIG. 28 illustrates a block diagram of a system 2800 for detecting events based on updates to node profiles from electronic activities. As shown in FIG. 28, an electronic activity 2852 is associated with an electronic activity identifier 2854 (“EA-098”). The electronic activity 2852 can include corresponding metadata, as described above, a body, and a respective signature 2860 included in the body of the electronic activity 2852. The activity field-value pairs for electronic activity 2852 can include a sender FirstName: John and LastName: Smith, Company: NewCo, Title: CEO, Work Phone No: 212-1212, Cell Phone No.: 617-555-2000. The node graph generation system 200, a part of the data processing system 9300, can ingest the electronic activity 2852 to create and update node profiles.

As illustrated in FIG. 28, the system 2800 includes a first representation 2862a of a node profile corresponding to NPID-12. The first representation 2862a of the node profile was generated or updated before the electronic activity 2852 was ingested by the node graph generation system 200. The first representation 2862a of the node profile can include one or more field-value pairs, where a node value is associated with a node field. For example, the first representation 2862a of the node profile includes node field-value pairs First Name: John, First Name: Johnathan, Last Name: Smith, Email: J@acme.com, Title: Director, Title: CEO, Company Name: Acme, Work Phone No.: 555-555-5439, Cell Phone No.:617.555.2000. Each node value of the node field-value pairs is associated with a confidence score. The data processing system 9300 can derive a true state of the respective node profile based on the confidence score of a certain value. The confidence score can represent an expectation or likelihood that the respective node value represents a true state of the node profile at any given time. The first representation 2862a of the node profile includes a first state 2870 of the node profile. The first state 2870 can correspond to a node field-value pair of the node profile. For example, the first state 2870 can include or be indicative of a node field-value pair associated with NPID-12 before the data processing system 9300 ingests electronic activity EA-098 (e.g., corresponding to a time stamp that is prior to a time stamp of when EA-098 was transmitted or received). The first state 2870 can correspond to the entire node profile or a tag associated with the node profile. For example, the first state 2870 can include or be indicative of multiple node field-value pairs associated with NPID-12 before the data processing system 9300 ingests electronic activity EA-098. The first representation 2862a of the node profile can be updated to additional representations of the node profile.

The system 2800 includes a second representation 2862b of a node profile corresponding to NPID-12. The second representation 2862b of the node profile was updated after the electronic activity 2852 was ingested by the node graph generation system 200. The second representation 2862b of the node profile can include one or more field-value pairs, where a node value is associated with a node field. For example, the second representation 2862b of the node profile includes node field-value pairs First Name: John, First Name: Johnathan, Last Name: Smith, Email: J@acme.com, Email: J@NewCo.com, Title: Director, Title: CEO, Company Name: Acme, Company Name: NewCo, Work Phone No.: 555-555-5439, Work Phone No.: 555.212.1212, Cell Phone No.: 617.555.2000. Each node value of the node field-value pairs is associated with a confidence score. The data processing system 9300 can derive a true state of the respective node profile based on the confidence score of a certain value. The second representation 2862b of the node profile includes a second state 2872 of the node profile. The second state 2872 can correspond to a node field-value pair of the node profile. The second state 2872 can correspond to the entire node profile or a tag associated with the node profile. The state change can include a tag assigned to the node profile. The state change can include an update regarding one or more node values. The node values can be associated with a node field of the node field-value pair. The data processing system 9300 can use the state change to detect events. The data processing system 9300 can detect the state change by identifying a difference between the second state 2872 and the first state 2870. As an example, the data processing system 9300 can detect the state change based on an additional value added to an existing field of a node profile. For example, J@NewCo is an added value to the Email field of the node profile NPID-12 for the second state 2872, which was not assigned to the Email field of the first state 2870. The state change can include a new value added to a new field of a node profile. The state change can include an increase in the confidence score of a value of a field of a node profile, such as if the data processing system 9300 determines, responsive to determining or updating the confidence score of each value, that an order of the confidence scores of the values has changed. The data processing system 9300 can detect the state change and related event conditions and event types using a table 2812 as described below.

As illustrated in FIG. 28, the system 2800 includes a table 2812 showing a relationship between node fields, event conditions and event types. The data processing system 9300 can use the table 2812 to retrieve event conditions based on detecting state changes corresponding to one or more fields of the node profile, and can use the table 2812 to retrieve event types corresponding to event conditions that are determined to be satisfied by the state change. For example, responsive to detecting a state change corresponding to the Title field, the data processing system 9300 can perform a lookup to retrieve the event condition corresponding to the Title field to be a new value event condition, determine if the state change was a new value assigned to the Title field, and responsive to determining that the state change was a new value assigned to the Title field, determine whether the event type of the state change was a promotion or a demotion (e.g., based on a table indicating a hierarchy of title values maintained by the data processing system 9300). The table 2812 can include a Node Field column, an Event Condition column, and an Event Type column. In some embodiments, the data processing system 9300 can determine that a state change satisfies an event condition (e.g., new value, bounce back). A new value can indicate that the data processing system 9300 generated a new value for a node field of a node profile. A bounce back can indicate that the data processing system 9300 determines that the electronic activity is a bounce-back electronic activity. As discussed above, the data processing system 9300 can identify a difference between the first state and the second state to detect the state change. The data processing system 9300 can detect the state change based on detecting a tag assigned to the first node profile. The state change can correspond to the Event Condition column of table 2812. The Event Condition column of table 2812 can include new value, bounce back, among others. For example, the state change can include a bounce back tag of the Event Condition column of table 2812. The event condition can correspond to a node field (e.g., Title, Last Name, Company Name). The Node Field column of table 2812 can include Title, Company Name, Last Name, Email, among others. The data processing system 9300 can store an association between the first node profile and an event type (e.g., promotion, move, marriage, demotion) corresponding to the event condition.

The Event Type column of table 2812 can include Promotion, Move, Marriage, among others. The node field can correspond to one or more event conditions. The node field can correspond to one or more event types. Additional details related to the functions performed by the data processing system 9300 to store the association between node profiles and event type are provided herein.

In further detail, the data processing system 9300 can detect the association between node profiles and event type by extracting the context of electronic activities and vacations from calendars, electronic activity responses, out of office messages, among others. For example, to detect an event type corresponding to Promotion, the data processing system 9300 can detect an increase in the number of electronic activities containing praise for an achievement or good wishes on a special occasion. The data processing system 9300 can parse words such as “congratulations”, “congrats”, “well done”, among others, to confirm an event type corresponding to Promotion. The data processing system 9300 can detect an event type corresponding to Promotion by detecting a change in the signature of an electronic activity. The data processing system 9300 can detect an event type corresponding to Promotion by detecting a new title in the signature of the electronic activity. To detect an event type corresponding to Marriage, the data processing system 9300 can extract information from an autoresponder or automatically generated responses that contain information about a vacation or honeymoon. The data processing system 9300 can detect a date of the marriage by parsing the context of electronic activities around the time of a name change. The data processing system 9300 can detect the date of the marriage based on the timestamp of an out of office message.

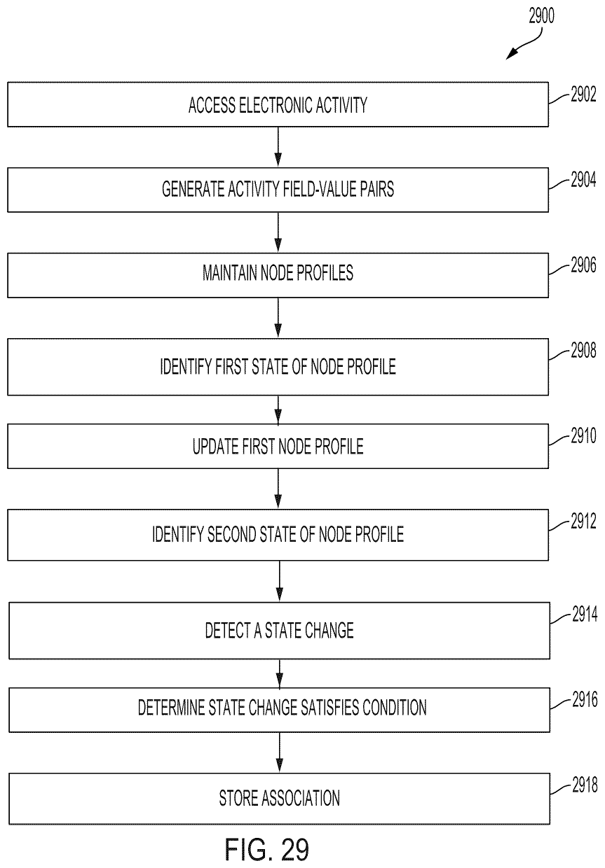

FIG. 29 illustrates a method 2900 for detecting events based on updates to node profiles from electronic activities. Briefly, the data processing system 9300 can access an electronic activity transmitted or received via an electronic account associated with a data source provider (BLOCK 2902). The data processing system 9300 can generate a plurality of activity field-value pairs (BLOCK 2904). The data processing system 9300 can maintain a plurality of node profiles (BLOCK 2906). The data processing system 9300 can identify a first state of a first node profile (BLOCK 2908). The data processing system 9300 can update the first node profile (BLOCK 2910). The data processing system 9300 can identify a second state of the first node profile (BLOCK 2912). The data processing system 9300 can detect a state change of the first node profile (BLOCK 2914). The data processing system 9300 can determine that the state change satisfies an event condition (BLOCK 2916). The data processing system 9300 can store an association between the first node profile and an event type corresponding to the event condition (BLOCK 2918).

The method for detecting events based on updates to node profiles from electronic activities can be executed by the data processing system 9300. As described herein, the data processing system 9300 can include the electronic activity ingestor 205, the electronic activity parser 210, and the node profile manager 220, among other components that make up the data processing system 9300.

Functionality relating to each of these components for generating new node profiles using information from electronic activities is further provided below.

In further detail, the data processing system 9300 can access an electronic activity transmitted or received via an electronic account associated with a data source provider (BLOCK 2902). In some embodiments, the electronic activity ingestor 205 is configured to ingest or access a plurality of electronic activities transmitted or received via electronic accounts associated with one or more data source providers. The electronic activity ingestor 205 can access electronic activities via one or more servers hosting or storing the electronic activities. The servers can store electronic activities transmitted from or received by accounts corresponding to an enterprise. For instance, the servers can be mail servers, phone log servers, calendar servers or any other entity that can store emails, calendar events, phone logs, or other electronic activities of accounts associated with an enterprise, such as a company. Referring to FIG. 28, the electronic activity ingestor 205 can access the electronic activity 2852. The electronic activity 2852 can be accessed from a mail server of a first data source provider corresponding to the sender of the electronic activity 2852. In some embodiments, the electronic activity 2852 can be accessed from one or more electronic activity accounts corresponding to the sender of the first electronic activity 2852.

Responsive to the electronic activity ingestor 205 ingesting the electronic activities, the data processing system 9300 can process the electronic activity. The data processing system 9300 can process the electronic activity by parsing the contents of the electronic activity and using the parsed electronic activity to assign tags to the electronic activities. The data processing system 9300 can link the electronic activities to one or more of the node profiles generated or maintained by the node profile manager 220. The data processing system 9300 can process the electronic activities by generating activity field-value pairs.

The data processing system 9300 can generate a plurality of activity field-value pairs (BLOCK 2904). In some embodiments, the electronic activity parser 210 can generate the plurality of activity field-value pairs from an electronic activity of the plurality of electronic activities. Each activity field-value pair can include a data structure that associates a particular field to a value for the field that the data processing system 9300 extracts from the electronic activity. The activity field-value pair can include an activity value associated with an activity field. For example, the data processing system 9300 can generate a field-value pair associating a value of “John” to the First Name field, a value of “Smith” to the Last Name field, a value of “CEO” to the Title Field, and a value of “Acme” to the Company Name based on the electronic activity 2852 illustrated in FIG. 28. The data processing system 9300 can generate multiple activity-field value pairs from each electronic activity (e.g., multiple first name-value field pairs based on information from a sender field and a signature block).

The data processing system 9300 can maintain a plurality of node profiles (BLOCK 2906).

The data processing system 9300 can maintain the plurality of node profiles in one or more data structures. The one or more data structures can be stored in a memory coupled to the one or more processors. Each node profile of the plurality of node profiles can include one or more node field-value pairs. Each node field-value pair can include a node value associated with a node field. Each node value can be associated with a confidence score. The confidence score can represent an expectation or likelihood that the respective node value represents the state of the node profile at a given point in time. In some embodiments, the node profile manager 220 is configured to maintain a plurality of node profiles. As described above, these node profiles can be generated from electronic activities and the information included in such electronic activities. In some embodiments, the node profiles can include values that are determined from one or more systems of record accessible to the node graph generation system 200. As shown in FIG. 28, node profile NPID-12 is maintained by the node profile manager 220 and populated or updated from information included in the electronic activities, such as EA-098 that is ingested by the data processing system 9300.

The data processing system 9300 can identify a first state of a first node profile (BLOCK 2908). The first state can correspond to a first node field-value pair of the one or more field-value pairs of the first node profile. For example, and referring to FIG. 28, the data processing system 9300 can identify a first state 2870 of a first representation 2862a of a node profile corresponding to NPID-12. The first state 2870 of the first representation 2862a the first node profile can correspond to a node field-value pair, Company Name: Acme. The data processing system 9300 can identify the first state 2870 of the first node profile before the ingestor 205 ingested electronic activity 2852. The data processing system 9300 can identify the first state 2870 corresponding to other node field-value pairs of the first node profile. For example, the first state 2870 of the first representation 2862a of the node profile can correspond to a node field-value pair, Work Phone No: 555.555.5439. The data processing system 9300 can change the first state 2870 of the first node profile by updating the first node profile.

The data processing system 9300 can update the first node profile (BLOCK 2910). The data processing system 9300 can update the first node profile using an electronic activity. For example, and referring to FIG. 28, the data processing system 9300 can update the first node profile corresponding to NPID-12 based on electronic activity 2852. The data processing system 9300 can add a node field-value pair to the first node profile corresponding to NPID-12. For example, the data processing system 9300 can add the node field-value pairs, Email: J@NewCo.com, Company Name: NewCo and Work Phone No: 555.212.1212 to the first node profile.

The data processing system 9300 can identify a second state of the first node profile (BLOCK 2912). The data processing system 9300 can identify the second state subsequent to updating the first node profile using the electronic activity. The data processing system 9300 identify the second state responsive to updating the first node profile using the electronic activity. The second state can correspond to a first node field-value pair of the one or more field-value pairs of the first node profile. For example, the second state can include a representation of at least a portion of the data of the one or more field-value pairs of the first node profile at a time (e.g., time stamp) for which the data processing system 9300 identifies the second state. The data processing system 9300 can generate each node profile state using values corresponding to data points having timestamps prior to or contemporaneous with the corresponding point in time. In some embodiments, the data processing system 9300 generates the node profile state by filtering the data points used to support the values of the node profile based on the timestamp corresponding to node profile state. For example, the data processing system 9300 can identify a point in time for which the node profile state is to be generated, compare each timestamp of each data point to the identified point in time, include each data point having a timestamp prior to or contemporaneous with the identified point in time in an output of the filter, and exclude (e.g., filter out) each data point having a timestamp after the identified point in time. For example, and referring to FIG. 28, the data processing system 9300 can identify a second state 2872 of a second representation 2862b of the first node profile corresponding to NPID-12. The first state 2870 and second state 2872 of the first node profile can correspond to a node field-value pair, Company Name: NewCo. The data processing system 9300 can identify the second state 2872 corresponding to other node field-value pairs of the first node profile. For example, the second state 2872 of the first node profile can correspond to a node field-value pair, Work Phone No: 555.212.1212 or Email: J@NewCo.com. The data processing system 9300 can identify the second state 2872 of the first node profile after the ingestor 205 ingested electronic activity 2852.

The data processing system 9300 can detect a state change of the first node profile (BLOCK 2914). The data processing system 9300 can detect the state change based on the first state and the second state. For example, the data processing system 9300 can identify the differences or similarities between the first state and the second state to detect the state change. The data processing system 9300 can detect a state change by detecting that a tag has been assign to the first node profile. For example, the data processing system 9300 can detect an electronic activity tagged as a bounce back electronic activity. The data processing system 9300 can use the bounce back electronic activity to assign a negative contribution score to reduce the confidence score of a value. For example, and referring to FIG. 28, the data processing system 9300 can assign a negative contribution score to j@acme to determine that j@acme is no longer with the company, Acme. The data processing system 9300 can detect a state change by detecting an update regarding one or more node values associated with the node field of the first node field-value pair. The data processing system 9300 can update the node profile by adding a new node field-value pair resulting from a value being added that was not previously associated with a field. The data processing system 9300 can update the node profile by adding a value to an existing node field-value pair for the value. The data processing system 9300 can identify that a value was added to the node profile in a specific way. For example, and referring to FIG. 28, the data processing system 9300 adds values for the email field, company name field, and work phone no. field to the node profile corresponding to NPID-12 based on parsing electronic activity 2852. The data processing system 9300 can detect a state change including the update to the Email, Company Name, and Work Phone No. fields. The data processing system 9300 can use the state change to determine if the state change satisfies an event condition.

The data processing system 9300 can determine that the state change satisfies an event condition (BLOCK 2916). The event condition can correspond to one or more predetermined node fields. The predetermined node fields can be of the one or more node field-value pairs of the first node profile. For example, and referring to FIG. 28, the data processing system 9300 can determine that one or more bounce back electronic activities from J@Acme.com satisfies a bounce back event condition corresponding to the Email field. The data processing system 9300 can receive bounce back electronic activity from multiple email domains to determine that the bounce back electronic activities satisfies the bounce back event condition. In this way, the data processing system 9300 can differentiate between bounce back electronic activity due to a deactivated email and bounce back electronic activity due to a disconnected server. The data processing system 9300 can determine that adding a new value to the Company Name field (e.g., NewCo) and receiving one or more bounce back electronic activities from J@Acme.com satisfies a bounce back and new value event condition corresponding to the Company Name field. The data processing system 9300 can determine that adding a new value with a confidence value exceeding a predetermined threshold to a field satisfies the event condition.

The data processing system 9300 can determine the event type based on rules triggering the event condition. For example, the data processing system 9300 can associate an event type corresponding to Moving (e.g., moving companies, leaving current employer) with bounce back electronic activities. The data processing system 9300 can determine that bounce back of electronic activities receive form one or more sources at one or more times corresponds to the event type for Moving. The data processing system 9300 can associate an event type corresponding to New Job with electronic activities or email identifying a new email address from the same entity. The data processing system 9300 can associate an event type corresponding to Marriage with electronic activities or email identifying a new name corresponding to the same email address. The data processing system 9300 can associate an event type corresponding to Marriage with electronic activities or email identifying a new name corresponding to a new email address of the entity associated with the old email address. The data processing system 9300 can associate an event type corresponding to Death with electronic activities or emails associated with condolences. The data processing system 9300 can associate an event type corresponding to Demotion with electronic activities or emails containing a lower ranking title. The data processing system 9300 can associate an event type corresponding to Promotion with electronic activities or emails containing a higher ranking title. The data processing system 9300 can maintain one or more data structures that maintain a list of titles. The data structures can also include a ranking assigned to each title of the list or some other indication that the system 9300 can use to determine a relative rank between two titles. In some embodiments, the system 9300 can include a machine learning model that can be trained to determine the relative rank between different titles.

The data processing system 9300 can store an association between the first node profile and an event type corresponding to the event condition (BLOCK 2918). Examples of event types can include company move, promotion, marriage, demotion, maternity/paternity leave, among others. The data processing system 9300 can store the association responsive to the state change satisfying the event condition. For example, and referring to FIG. 28, the data processing system 9300 can determine that a state change satisfying a bounce back event condition corresponding to the Email field is indicative of a person represented by NPID-12 no longer being at Acme. The data processing system 9300 can determine that a state change satisfying an added value event condition corresponding to the Company field is indicative of the person represented by NPID-12 moving to a new company (e.g., NewCo). The data processing system 9300 can determine that multiple state changes corresponding to multiple fields is indicative of an event type. Responsive to detecting the event condition, the data processing system 9300 can use the event condition to perform a lookup in table 2812. The data processing system 9300 can use the event condition associated with a particular node field in table 2812. The data processing system 9300 can retrieve the event type associated with the node field in table 2812 to store the association between the node profile and the event type.

In some embodiments, the data processing system 9300 can select the first node profile to update using the electronic activity based on a match score of the first node profile. The match score can indicate a likelihood that the electronic activity is transmitted or received by the electronic account corresponding to the first node profile. The match score can indicate a likelihood that the electronic activity is transmitted or received by a server corresponding to the electronic account. The data processing system 9300 can select the first node profile that has a match score that exceeds a predetermined threshold. Further, the node profile manager 220 can maintain a match score for each electronic activity of a particular node profile, or of each value of the node profile to which the electronic activity matched. By doing so, the node profile manager 220 can use the match score to determine how much weight to assign to that particular electronic activity. The data processing system 9300 can select the first node profile based on a match score that is above a predetermined threshold. The data processing system 9300 can select the first node profile based on one or more activity field-value pairs matching corresponding node field-value pairs.

In some embodiments, the node profile manager 220 can assign a first weight to electronic activities that have a first match score and assign a second weight to electronic activities that have a second match score. The first weight may be greater than the second weight if the first match score is greater than the second match score. In some embodiments, if no nodes are found to match the electronic activity or the match score between the email message and any of the candidate node profiles is below a threshold, the node profile manager 220 can be configured to generate a new node profile. The node profile manager 220 can then populate various fields of the new node profile from the information extracted from the electronic activity parser 210 after the electronic activity parser 210 parses the electronic activity.

In some embodiments, the data processing system 9300 can use confidence scores to determine that a state change satisfies an event condition. To use confidence scores to make the determination, the data processing system 9300 can update the first node profile. Updating the first node profile can include updating a second field-value pair corresponding to the first field using the electronic activity. The data processing system 9330 can identify a first field-value pair corresponding to the first field having a first highest confidence score in the first state. For example, and referring to FIG. 28, the data processing system 9300 can identify the first field-value pair Company Name: Acme having a first highest confidence score in the first state 2870. The data processing system 9300 can determine that a confidence score of the second field-value pair is greater than a corresponding confidence score of the first field-value pair in the second state. For example, the data processing system 9300 can determine that the second field-value pair Company Name: NewCo has a greater confidence score than Company Name: Acme in the second state 2872. The data processing system 9300 can identify the state change to be that the second field-value pair, which is not present in the first state 2870 (or has a relatively lower confidence score in the first state) has a greater confidence score than the first field-value pair in the second state 2872. By determining that the confidence score of the second field-value pair is greater than the corresponding confidence score of the first field-value pair in the second state, the data processing system 9300 can determine that the state change satisfies the event condition.

In some embodiments, the data processing system 9300 can use a tag to link or match a second electronic activity to a node profile. The data processing system 9300 can receive a second electronic activity (e.g., electronic activity 2852). The data processing system 9300 can identify the first node profile and at least one candidate node profile of the plurality of node profiles to which to link the second electronic activity. For example, the data processing system 9300 can identify the node profile corresponding to NPID-12. The data processing system 9300 can select the first node profile to which to link the second electronic activity. The data processing system 9300 can select the first node profile responsive to detecting the state change includes the tag assigned to the first node profile. A tag assigned to the first node profile can include an electronic activity tagged as a bounce back electronic activity. The tag can be indicative of the electronic activity being a bounce-back electronic activity. The data processing system 9300 can identify that a bounce back electronic activity resulted from an undeliverable email because the email address no longer belongs to an active person at the company.

In some embodiments, the data processing system 9300 can update a hierarchy data structure that includes the first node profile. The data processing system 9300 can make the update responsive to the state change satisfying the event condition. The hierarchy data structure can include a company cloud. The company cloud can identify a plurality of companies or enterprises. Each company included in the company cloud can be represented as a company or group node and each group node can include or be linked to one or member node profiles corresponding to people belonging to or affiliated with the company. The company cloud can refer to or include a group node graph or network of group nodes. A group node can be a representation of a company and include fields. Fields can include, for example, a company name, a company phone number, a company address, a unique identifier for the company, a company size, a company location, or other information associated with the company. The group node can further be linked to one or more member node profiles corresponding to people who are either employed by the company or in some embodiments, have some affiliation with the company.

Additional details on the company cloud are described at least in part in Section 11.

The data processing system 9300 can determine a field type of the first node field, such as to determine which event conditions to evaluate using the data of the first node field or state changes that relate to changes in values that are paired with the first node field. For example, the data processing system 9300 can use the field type of the first node field to determine if event conditions relating to a promotion, moving to a new company, or marriage. In some embodiments, the data processing system 9300 can determine that the first node field is at least one of a job title field, a company field, or a last name field. For example, and referring to FIG. 28, the first node field can include a job title or title field, a company or company name field, or a last name field. The update regarding one or more node values can indicate that the one or more node values include a new value associated with the first field in the second state and not in the first state. For example, and referring to FIG. 28, the data processing system 9300 can update the company name field to include a new value (“NewCo”) associated with the company name field in the second state 2872 and not in the first state 2870.

The data processing system 9300 can use the field type of the first node field to determine whether the entity (e.g., person) associated with the first node field has moved. In some embodiments, the data processing system 9300 can determine the first node field includes at least two of a city field, a state field, or a phone number field. Determining that the state change satisfies the event condition can include the first node field includes at least two of a city field, a state field, or a phone number field. For example, the first node field can include a city field and a phone number field, a city field and a state field, a state field and a phone number field, or a city field, a state field and a phone number field. The update regarding one or more node values can indicate that the one or more node values include a new value associated with the first field in the second state and not in the first state. For example, the update can indicate that the city field and the state field included new values which were included in the second state and not in the first state. The new values added to city field, state field or phone number field can indicate that a person has changed addresses or moved.

In some embodiments, the data processing system 9300 can store an association between the first node profile and the event type corresponding to the event condition and a time or a date. The data processing system 9300 can determine at least one of a time or a date at which the first electronic activity was transmitted or received. The data processing system 9300 can make the determination from the electronic activity. The electronic activity can include a timestamp including the time or date information. The data processing system 9300 can store the association between the first node profile and the event type corresponding to the event condition. Storing the association can include storing the association between the first node profile and the event type corresponding to the event condition and the at least one of the time or the date. For example, and referring to FIG. 28, the data processing system 9300 can determine a time or date when the electronic activity 2852 was transmitted or received. The data processing system 9300 can determine, based on the time or date when the electronic activity 2852 was transmitted or received, that the person associated with NPID-12 moved or changed companies. The data processing system 9300 can determine that the person associated with NPID-12 moved or changed companies on a date the electronic activity 2852 was transmitted or received.

The data processing system 9300 can detect an event condition to detect a certain event type. For example, the data processing system 9300 can process multiple electronic activities. The data processing system 9300 can detect an event condition has a confidence score above a threshold based on the multiple electronic activities. The data processing system 9300 can determine a date or time of the event type by identify a date or time of the first electronic activity indicating such an event. The data processing system 9300 can parse the electronic activity to determine the context and dates once the data processing system 9300 detects the event condition. For example, the data processing system 9300 can determine the date of a Promotion based on an electronic activity that describes the official announcement of the promotion. The data processing system 9300 can parse the electronic activity to determine the date or time of the event type corresponding to the Promotion.

### 21. Inferring Schedule Patterns Using Electronic Activities of Node Profiles

The present disclosure relates to systems and methods for inferring schedule patterns using electronic activities of node profiles. Although individuals that are employed may have scheduled work hours, due to technological advancements and the adoption of portable electronics, individuals may be performing work related activities outside of their scheduled work hours. At present, employers and others are unable to track an employee's actual work schedule due to the employee's ability to work remotely. As a result, it is hard to track an employee's performance or their availability at a given time period.

Moreover, some people may begin their workday at a different time than other people, even within the same enterprise or company. In a given day, a person may send a number of emails or engage in other electronic activity as a part of their employment. Where a person begins their workday earlier, the person may begin sending emails and engage in other electronic activity earlier than people who begin their work day later. Some employers may track schedules of employees. Typically, the employers rely on an employee punching a time card, logging into their work computer, or other time management system for tracking an employee's schedule. The systems and methods described herein are configured to access electronic activities generated by a user within a time period for inferring the user's schedule. The systems and methods described herein generate a temporal distribution of electronic activity for time intervals within the time period. The systems and methods described herein determine an electronic activity pattern based on the temporal distribution. The systems and methods described herein identify regions of the temporal distribution corresponding to an inferred schedule for the user based on a region identification policy.

As one example, a user, such as an employee, may initiate, transmit, respond to, or otherwise engage in electronic activity over a first period of time (e.g., from 9:00A-5:00P). Another user who works at the same enterprise or company as the first user may engage in electronic activity over a second period of time (e.g., from 10:00A-7:00P). The system may identify electronic activities engaged in by the users. For each user, the system may generate a temporal distribution of electronic activities for a time interval (such as a day, for instance). The system may determine an electronic activity pattern for the user based on the temporal distributions for the user. The system may identify a region of the time interval corresponding to a schedule of the user by applying a temporal distribution determination policy to the electronic activity pattern. The system may thus determine, identify, or otherwise infer a work schedule for the user based on electronic activity patterns for the user.

Furthermore, the system can determine the electronic activity pattern for the user based on electronic activities over a much larger period of time. For instance, to determine a daily schedule for the user, the system may rely on activities over a 90 day period of time. Doing so can help the system determine days that the user may be on vacation, traveling, at client visits, among others. Furthermore, the system can determine days when the employee is working extra hard or days when the employee is not as productive. By determining such performance related data of an employee, the employer or the employee can take appropriate actions based on this data. Moreover, the system can access data from one or more other systems to get more context relating to a schedule of an employee. It should be appreciated that the data from the one or more other systems can help identify if the user is on vacation or travelling or at a client site among others.

According to the embodiments of the systems and methods described herein, the system can parse electronic activity to determine a schedule for users. The system can assign, correlate, or otherwise associate the schedule with a node profile for the user. The system can leverage various information from various sources of electronic activity for generating a temporal distribution of electronic activity for each user within the company or enterprise. The system can identify an electronic activity pattern for each user based on a number of temporal distributions. The system can apply a temporal distribution determination policy to the electronic activity pattern for generating, determining, identifying, or otherwise inferring a work schedule for the user based on the electronic activity pattern. The company can thus determine employees schedules based on times in a given time interval in which employees are engaged in electronic activity, rather than relying on employees punching a time card, logging into their work computer, or other time management system for tracking an employee's schedule. Various other benefits and advantages of the present technical solution are apparent based on the description that follows.

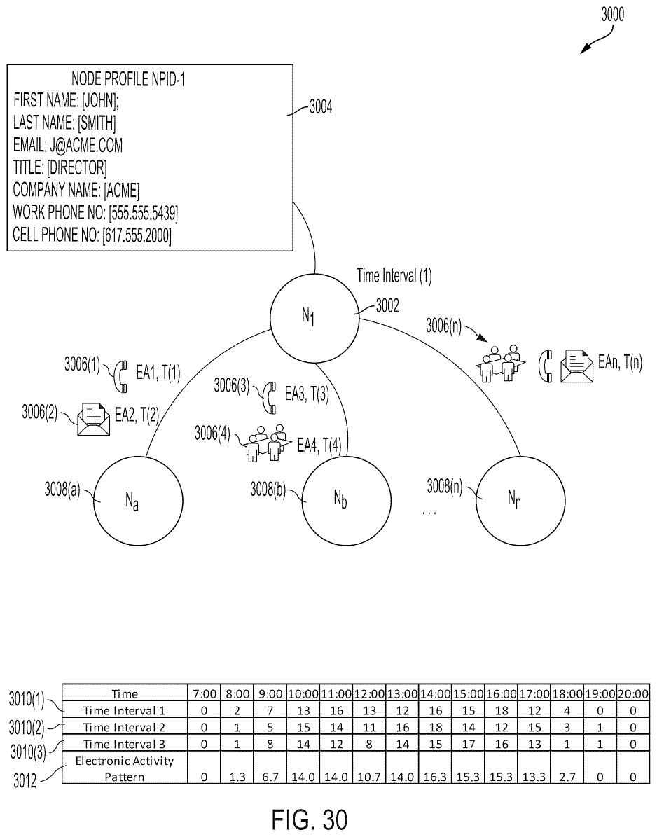

Referring now to FIG. 30, illustrated is a use case diagram 3000 of a user associated with a node profile 3004 engaged in a plurality of electronic activities 3006 in a time interval (e.g., within a day, a week, etc.). The user corresponding to node 3002 may engage in electronic activities 3006 with various users corresponding to other nodes 3008a-3008n within the time interval. Each user may include a corresponding node profile 3004 (e.g., a node profile 3004 corresponding to “John Smith”, and node profiles for each of the users who are participants with “John Smith” in electronic activities 3006(1)-3006(n)). As described in greater detail above, the system 200 may be configured to generate the node profiles 3004 based on electronic activity. In the use case diagram depicted in FIG. 30, a first user (e.g., “John Smith”) may generate and send a number of electronic activities at various times to various other users. The use case diagram shows a plurality of different types of electronic activities (e.g., phone calls, emails, meetings, etc.) conducted between John Smith and various other users. As described in greater detail below, the system 200 may be configured to determine, identify, or otherwise infer a work schedule for a user based on the electronic activities engaged in by user over the time interval.

The system 200 may be configured to detect, identify, register, collect, or otherwise access electronic activities 3006. Each electronic activity 3006 may be engaged in by at least one participant. In some instances, an electronic activity 3006 may include a plurality of participants. For instance, a user initiating an email to one recipient may be the sole participant of the electronic activity 3006 (e.g., the one participant being the sender of the email). As another example, a conference call, meeting, phone call, etc. may include two or more participants (e.g., members of the conference call, meeting, phone call, etc.). In some embodiments, the participants may change over time. For instance, where a phone call is initiated and not answered, the electronic activity 3006 may have one participant (e.g., the person initiating the phone call). Where the phone call is answered, the electronic activity 3006 may have two or more participants (e.g., the person initiating the phone call and the person(s) answering the phone call).

The system 200 may be configured to detect, receive, or otherwise identify electronic activities (such as electronic activity 3006) exchanged, transmitted, received, or otherwise engaged in by a user corresponding to a node profile. As described in greater detail above, the system 200 may include an electronic activity ingestor 205 which is configured to ingest electronic activities 3006 from a plurality of data source providers. The electronic activities 3006 may be received or ingested in real-time or asynchronously as electronic activities are generated, transmitted or stored by the one or more data source providers. The data source providers may be or include a server, which hosts a domain corresponding to one or more of the participants of the electronic activity (e.g., the sender or the recipient). The system 200 may identify electronic activities which identify a sender, one or more recipient(s), and include a body or content. As described in greater detail below, the electronic activity parser 210 of the system 200 may be configured to parse the identified electronic activities. The electronic activity parser 210 may parse the electronic activity to identify a timestamp for each electronic activity 3006 sent, generated, or otherwise initiated by a user associated with a node profile 3004. The system 200 may be configured to generate a temporal distribution for each time interval in a time period based on the timestamps. The system 200 may be configured to determine an electronic activity pattern based on the temporal distributions over the time period. The system 200 may be configured to identify a region of the temporal distribution corresponding to a schedule of the user by applying a region identification policy to the electronic activity pattern.

The system 200 may be configured to select, determine, locate, or otherwise identify electronic activity 3006 sent, received, or otherwise engaged in by the user corresponding to the node 3002. The system 200 may be configured to identify each electronic activity 3006 associated with the node profile 3004 for the user. The node profile 3004 may include a plurality of fields having values corresponding to accounts of the user. For instance, in the example depicted in FIG. 30, the node profile 3004 includes a value “J@ACME.COM” corresponding to a field associated with an email address, a value “555.555.5439” corresponding to a field associated with a work phone number, and a value “617.555.2000” corresponding to a field associated with a cell phone number. The electronic activity parser 210 may parse each electronic activity to identify a set of electronic activities in which John Smith was a participant based on which electronic activities indicate a sender or recipient having a value with matches at least one of the values associated with a field of the node profile 3004 (e.g., a phone call dialed by or answered via a phone number having a value which matches the value corresponding to the field associated with work phone number or cell phone number, an email sent via an email address having a value which matches the value corresponding to the field associated with the email address, and so forth).

The system 200 may parse each of the electronic activities 3006 sent, received, or otherwise engaged in by the user corresponding to the node 3002. In some embodiments, the electronic activity parser 210 may parse the header or metadata for the electronic activity 3006. The electronic activity parser 210 may parse the header or metadata for the electronic activity 3006 to identify a timestamp associated with each electronic activity 3006. As stated above, each electronic activity 3006 may include a corresponding timestamp. The timestamp may be or indicate the time at which the electronic activity 3006 was generated, sent, received, stored, etc. The electronic activity parser 210 may be configured to parse each electronic activity 3006 to identify the timestamp associated with each respective electronic activity.

In some embodiments, the system 200 may be configured to determine a type of electronic activity for each electronic activity 3006 sent, received, or otherwise engaged in by the user corresponding to the node 3002. In some embodiments, the tagging engine 265 may be configured to tag each electronic activity 3006 with a tag corresponding to the type of electronic activity. For instance, the tagging engine 265 may tag an electronic activity 3006 as an email where the electronic activity 3006 was transmitted via an electronic account included as a value of an email address field of the node profile 3004. The system 200 may determine the type of electronic activity for each electronic activity 3006 sent, received, or otherwise engaged in by the user corresponding to the node 3002 based on the tag for each electronic activity. As another example, the electronic activity parser 210 may be configured to determine the type of electronic activity for each electronic activity 3006. As described above, the electronic activity parser 210 may parse electronic activity to identify a set of electronic activities in which a user was engaged based on which electronic activities indicate a sender or recipient having a value with matches at least one of the values associated with a field of the node profile 3004. In some embodiments, the electronic activity parser 210 may store an association between each field of the node profile 3004 and a corresponding type of electronic activity. The electronic activity parser 210 may identify the type of electronic activity for each electronic activity in the set based on the association between the field of the node profile 3004 and which value was used for including the electronic activity 3006 in the set. For example, the electronic activity parser 210 may store an association between the field “EMAIL” and the type of electronic activity “EMAIL”. The electronic activity parser 210 may identify electronic activities for including in the set of electronic activities linked to the node profile 3004 based on which electronic activities were sent or received by “J@ACME.COM.” The electronic activity parser 210 may similarly identify the type of each electronic activity in the set as an email based on which electronic activities which were sent or received by “J@ACME.COM” and the association with the corresponding field “EMAIL” and type of electronic activity “EMAIL.”

In some embodiments, the electronic activity parser 210 may be configured to parse the electronic activity 3006 to determine a location corresponding to the user when the user engaged in the electronic activity. The electronic activity 3006 may include metadata corresponding to an IP address of the client device which was used for generating, transmitting, sending, or otherwise engaging in the electronic activity 3006. As another example, the electronic activity 3006 may include metadata corresponding to a network address, network name, etc. to which the client device was connected when the user was engaging in the electronic activity. The electronic activity parser 3006 may parse the metadata to extract such information for identifying, determining, or otherwise inferring a location of the user when the user engaged in the electronic activity. The electronic activity parser 210 may maintain, include, or otherwise access a list, ledger, database, etc. of IP addresses of enterprise client devices, enterprise network addresses, enterprise network names, etc. associated with the node profile 3004 for the user. The electronic activity parser 210 may compare the data extracted from the electronic activity to the data included in the database to determine whether the electronic activity was engaged in on the enterprise client device of the user, the enterprise network, etc. Where the electronic activity parser 210 determines the IP address, network address, network name, etc. matches the data in the database, the electronic activity parser 210 may determine the electronic activity was engaged in at a location of the enterprise. Where the electronic activity parser 210 does not identify a match, the electronic activity parser 210 may determine the electronic activity was engaged in at a location other than the location of the enterprise. The electronic activity parser 210 may determine, identify, or infer the location in which the user engaged in the electronic activity for applying weights to the electronic activity in generation of an electronic activity pattern for the user.

In some embodiments, the electronic activity parser 210 may be configured to parse the electronic activities 3006 to identify or infer a complexity of the electronic activity 3006. The electronic activity parser 210 may parse the electronic activities 3006 to identify a language complexity score of the electronic activity. The language complexity score may indicate a level of language complexity. The electronic activity parser 210 may parse the electronic activity to identify an average number of syllables per word, a number of words present in each sentence, etc. The electronic activity parser 210 may use the average number of syllables per word, word choices, grammar complexity, and/or number of words present in each sentence to generate the language complexity score. The language complexity score may be a score on any number of scales. For instance, the language complexity score may be on the Flesch Reading Ease scale, which calculates the readability of content on a scale ranging between 0-100 (with lower numbers indicating content is more difficult to comprehend). As another example, the language complexity score may be on the Flesch-Kincaid Grade scale, which indicates how many years of education needed to comprehend the content. In some embodiments, the electronic activity parser 210 may identify or infer the complexity of the electronic activity 3006 by determining a character count or word count (2910). The electronic activity parser 210 count each character in the body (e.g., letters, numbers, punctuation, spaces, etc.). The electronic activity parser 210 may group character(s) together which are separated by a space or spaces, and count each group as one word. The electronic activity parser 210 may count the words/characters within the electronic activity. The electronic activity parser 210 may determine, identify, or infer the complexity of electronic activity applying weights to the electronic activity in generation of an electronic activity pattern for the user.

The system 200 may be configured to arrange, sort, filter, compile, or otherwise identify the electronic activities 3006 generated by the user within a time interval. In some embodiments, the system 200 may include a temporal distribution generation engine. The temporal distribution generation engine may be or include any script, file, program, application, set of instructions, or computer-executable code that is configured to determine, identify, compile, produce, or otherwise generate a temporal distribution of electronic activity for a user within the time interval. The time interval may be, for instance, a day (e.g., a 24 hour period), a week (e.g., a working week from Monday through Friday), and so forth. In some implementations, the temporal distribution engine may be embodied on or otherwise be a component or element of the electronic activity parser 210. The temporal distribution generation engine may be configured to generate the temporal distribution of electronic activity for a series of time intervals within a time period (e.g., within a month window, a 90 day window, a 180 day window, and so forth). Hence, a time period may be made up of a group of time intervals.

The temporal distribution generation engine may be configured to generate the temporal distribution of electronic activity based on the identified timestamps for each of the electronic activity in the set of electronic activity for a user. The electronic activity parser 210 may be configured to identify the timestamp for each electronic activity generated, sent, received, or otherwise engaged in by the user. The temporal distribution generation engine may be configured to generate the temporal distribution by compiling each electronic activity engaged in by the user having a timestamp which falls within a time interval. The temporal distribution generation engine may sort, compile, or otherwise filter each of the electronic activities within the set of electronic activities by timestamp. The temporal distribution generation engine may maintain, for each time interval within the time period, a subset of electronic activities having timestamps which fall within the respective time intervals. Hence, a given time interval may have any number of electronic activities based on which electronic activities were engaged in by the user within that time interval.

Continuing the example shown in FIG. 30, John Smith engaged in two electronic activities (a phone call EA1 and an email EA2) with a second node (e.g., N2 at T(1) and T(2), respectively), two electronic activities (a phone call EA3 and a meeting EA4) with a third node (e.g., N3 at T(3) and T(4), respectively). John Smith may engage in any number of electronic activities at various times within a first time interval (e.g., Time Interval (1)). The temporal distribution generation engine may compile each of the electronic activities engaged in by John Smith for the first time interval based on the timestamps (e.g., T(1)-T(N)) which fall within the time interval (e.g., Time Interval (1)). The temporal distribution generation engine may sort each of the electronic activities compiled for the first time interval by their respective timestamps.

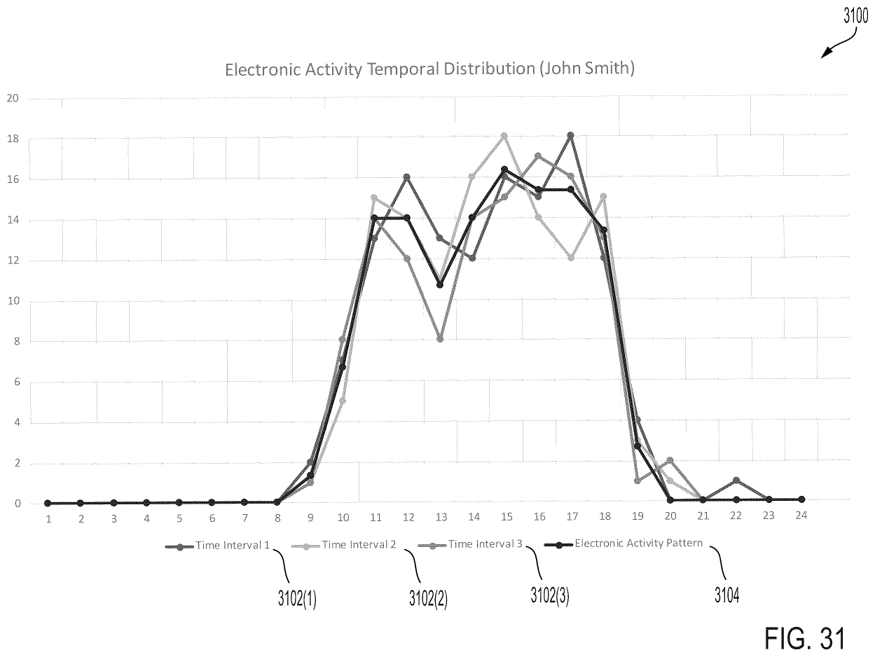

Referring now to FIGS. 30 and 31, the temporal distribution generation engine may compile, produce, or otherwise generate a graphical representation 3102 of the temporal distributions. Specifically, FIG. 31 depicts a graph 3100 showing a plurality of graphical representations 3102 of temporal distributions 3010 of electronic activity at various time intervals. The temporal distribution generation engine may generate a temporal distribution 3010(1) of electronic activities for the first time interval (Time Interval (1)). In some implementations, each electronic activity within the subset of electronic activities falling within the first time interval may be arranged within a time frame (e.g., an hour) of the first time interval (e.g., a 24 hour period). The temporal distribution generation engine may generate the temporal distribution 3010(1) by grouping each electronic activity in the subset by which time frame of the first time interval the respective timestamps fall within. As shown in FIG. 30, for the first time interval (Time Interval (1)), John Smith may not have engaged in any electronic activity (e.g., did not send, initiate, or otherwise actively participate in any electronic activity) between time frames beginning at 00:00-07:00. However, in the example shown in FIG. 30, John Smith engaged in two electronic activities (for instance, participated in a call, responded to an email, etc.) within the time frame between 8:00-8:59, seven electronic activities within the time frame between 9:00-9:59, 13 electronic activities within the time frame between 10:00-10:59, and so forth through 18:00-18:59 (e.g., 6:00P-6:59 PM). Following 18:59, John Smith may not have engaged in any electronic activity for the rest of the time interval (e.g., from 19:00-23:59). The graphical representation 3102(1) corresponding to the temporal distribution 3010(1) shows the electronic activities generated, initiated, participated in, or otherwise engaged in by the user (e.g., John Smith) over the first time interval. As shown in FIG. 31, the temporal distribution generation engine may generate graphical representations 3102(1)-2902(3) of the temporal distributions 3010(1)-2810(3) of electronic activity at various time intervals (Time Interval (1)Time Interval (3)).

The temporal distribution generation engine may be configured to generate, for each time interval, a temporal distribution 3010. While charts representing three temporal distributions are depicted in FIG. 30 and FIG. 31, it is noted that the temporal distribution generation engine may generate any number of temporal distributions 3010 depending on the number of time intervals within the time period. In some implementations, the temporal distribution generation engine may generate temporal distributions 3010 for time intervals corresponding to business days. The temporal distribution generation engine may maintain, include, or otherwise access a list of business days corresponding to an enterprise associated with the user. The list of business days may be, for instance, Monday through Friday. In some implementations, the list of business days may be a preset list of business days. In some embodiments, a person associated with the enterprise, such as an administrator, the user, etc., may update, modify, or otherwise change the preset list of business days based on scheduled non-business days (e.g., holidays), days off, and so forth. In some embodiments, the temporal distribution generation engine may be configured to automatically detect (e.g., based on deviations from an electronic activity pattern for the user) non-business days. The temporal distribution generation engine may generate temporal distributions for each time interval which corresponds to a business day. The temporal distribution generation engine may forego generation of (or disregard, delete, or otherwise remove already generated) temporal distributions 3010 of electronic activity corresponding to non-business days.

The system 200 may be configured to generate, identify, or otherwise determine an electronic activity pattern for the user. In some embodiments, the system 200 may include a pattern determination engine. The pattern determination engine may be or include any script, file, program, application, set of instructions, or computer-executable code that is configured to generate, identify, compile, produce, or otherwise determine an electronic activity pattern for the user within the time period. The pattern determination engine may determine the electronic activity pattern based on each of the temporal distributions 3010 generated by the temporal distribution generation engine that fall within the time period. The pattern determination engine may determine the electronic activity pattern by applying a pattern determination policy to each of the temporal distributions within the time period. The pattern determination policy may be a rule-based policy, a probabilistic model, etc. The pattern determination policy may be generated by an administrator defining the rules, generated via a machine learning algorithm using a training set of temporal distributions and corresponding electronic activity patterns, and so forth.

The pattern determination engine may be configured to apply the pattern determination policy to each of the temporal distributions within the time period for generating the electronic activity pattern for the user. As described in greater detail below, the system 200 may be configured to determine a region of the time interval corresponding to a schedule of the user based on the electronic activity pattern for the user.

The pattern determination engine may use the pattern determination policy to compute an average, mean, etc. of electronic activity for each time frame within a respective time interval in the time period. In some embodiments, the pattern determination engine may use the pattern determination policy to may filter one or more data outliers in the temporal distribution (e.g., a number of electronic activities exceeding a standard deviation from the average or mean of electronic activity within a respective time frame). In some embodiments, the pattern determination engine may use the pattern determination policy to may filter electronic activities within a respective time frame having a mean or average less than one electronic activity. Continuing the example depicted in FIG. 30, the pattern determination engine may apply the pattern determination policy to each of the temporal distributions 3010(1)-2810(3) to identify an electronic activity pattern 3012. The electronic activity pattern 3012 may be an average of electronic activity within each time frame (e.g., 8:00-19:59) for each time interval (Time Interval (1)-Time Interval (3)). The pattern determination engine may filter, from the electronic activity pattern 3012, data points for time frames with average electronic activity less than one (e.g., time frame 19:00-19:59 has an average electronic activity of 0.66, which is less than one). The pattern determination engine may be configured to generate a graphical representation 3104 of the electronic activity pattern 3012 on the graph 3100. As can be seen in FIG. 31, the electronic activity pattern 3012 may track, trend, or otherwise follow the temporal distributions of electronic activity for each time interval within the time period.

In some implementations, the pattern determination engine may use the pattern determination policy to leverage data from the electronic activity for applying weights to different electronic activities within the respective temporal distributions. In some embodiments, the pattern determination engine may use the pattern determination policy to apply weights to electronic activity based on the determined type of electronic activity, the complexity of electronic activity, and/or the location in which the electronic activity was engaged.

The pattern determination engine may use the pattern determination policy to apply weights to electronic activity within the temporal distributions based on the determined type of electronic activity. The pattern determination engine may use the pattern determination policy to use the identified type of electronic activity within the time interval for assigning weights to each electronic activity. In such embodiments, the weights may be indicative of, correspond to, or otherwise associated with a level of engagement of the user in engaging in the electronic activity. For instance, the pattern determination engine may use the pattern determination policy to apply weights that favor electronic activity corresponding to participating in meetings greater than electronic activity corresponding to emails. As another example, the pattern determination engine may use the pattern determination policy to apply weights that favor electronic activity corresponding to phone calls or conference calls greater than electronic activity corresponding to emails. The pattern determination engine may use the pattern determination policy to generate the electronic activity pattern as described above following applying weights to the respective electronic activities in each temporal distribution based on the type of electronic activity.

The pattern determination engine may use the pattern determination policy to apply weights to the electronic activity based on the complexity of each electronic activity. The pattern determination engine may use the pattern determination policy to use the identified complexity of electronic activity within the time interval for assigning weights to each electronic activity. In such embodiments, the weights may be indicative of, correspond to, or otherwise associated with a level of engagement of the user in engaging in the electronic activity. The pattern determination engine may use the pattern determination policy to leverage the language complexity score and/or word/character count generated, determined, or otherwise identified by the electronic activity parser 210 for applying weights to the electronic activity. The pattern determination engine may use the pattern determination policy to include, maintain, or otherwise access a database or set of rules corresponding to language complexity scores, word or character counts, etc. and associated weights. For instance, the pattern determination engine may use the pattern determination policy to apply weights which favor electronic activity having a higher complexity score, higher word or character count, etc. Hence, the weights may increase with the language complexity score, word count, character count, etc. As the language complexity score, word count, character count, etc. increases, the level of engagement of the user in engaging in the electronic activity may be presumed, assumed, or inferred to correspondingly increase. The pattern determination engine may use the pattern determination policy to generate the electronic activity pattern as described above following applying weights to the respective electronic activities in each temporal distribution based on the complexity of electronic activity.

The pattern determination engine may use the pattern determination policy to apply weights to the electronic activity based on the location in which the electronic activity was generated. The pattern determination engine may use the pattern determination policy to use the location in which the electronic activity was generated, sent, or otherwise engaged in by the user for assigning weights to each electronic activity. For instance, the pattern determination engine may use the pattern determination policy to apply weights which favor electronic activity engaged in at a location associated with an enterprise corresponding to the user. As another example, the pattern determination engine may use the pattern determination policy to apply weights which disregard, discard, filter, or otherwise ignore electronic activity generated at a location other than a location associated with the enterprise. The pattern determination engine may use the pattern determination policy to generate the electronic activity pattern as described above following applying weights to the respective electronic activities in each temporal distribution based on the location in which the user was engaged in the electronic activity.

The system 200 may be configured to determine, select, or otherwise identify a region of the electronic activity pattern. In some embodiments, the system 200 may include a region identification engine. The region identification engine may be or include any script, file, program, application, set of instructions, or computer-executable code that is configured to generate, determine, compile, select, or otherwise identify a region of the electronic activity pattern for the user. The region may be associated with or otherwise correspond to an inferred schedule of the user. The region identification engine may apply a region identification policy to the electronic activity pattern for identifying the region. The region identification engine may apply the region identification policy to the electronic activity pattern for determining which portions of the time interval the user is usually, normally, typically, etc. engaged in electronic activity. The system 200 may identify the region for inferring a work schedule of the user with the work schedule being correlated with the time interval in which the user is typically engaged in electronic activity.

In some embodiments, the region identification policy may be or include a probabilistic model. The probabilistic model may be generated via a machine learning algorithm trained based on a plurality of electronic activity patterns and corresponding work schedules. The machine learning algorithm may receive, as an input, a plurality of electronic activity patterns for different users and their respective work schedules (which may be manually entered, for instance.). The machine learning algorithm may output a probabilistic model which is configured to receive a given electronic activity pattern for a user and output a corresponding region of a time interval which is likely to correspond to a work schedule for the user. The region identification policy may refine the probabilistic model over time as subsequent work schedules are verified. The region identification policy may update the probabilistic model by retraining the probabilistic model via the machine learning algorithm.

In some embodiments, the region identification policy may leverage thresholds of electronic activity within a time frame of the time interval for identifying the region. The region identification policy may include, maintain, or otherwise access a first threshold corresponding to a first time of the time interval and a second threshold corresponding to a second time of the time interval. The region identification policy may be used by the system to apply the first and second thresholds to the electronic activity pattern to identify the first and second time of the time interval. In some embodiments, the threshold may be or correspond to a change in electronic activity over a time frame. In some embodiments, the thresholds may correspond to an average amount of electronic activity over two adjacent time frames. In some embodiments, the thresholds may correspond to a slope of the graphical representation 3104 of the electronic activity pattern. The region identification policy may be used by the system to apply the first and second thresholds to the electronic activity pattern to identify a first time corresponding to an uptick in electronic activity and a second time corresponding to a downtick in electronic activity engaged in by the user. The region identification policy may be used by the system to select the region of the time interval as spanning the first and second time.

In some embodiments, the region identification policy may be used by the system to compare the electronic activity pattern to one or more electronic activity patterns stored in a database or data structure and associated with respective regions of the time interval. The database may include electronic activity patterns and respective regions of the time interval similar to the datasets used to train the machine learning algorithm for producing the probabilistic model as described above. The region identification policy may be used by the system to compare the electronic activity pattern of the user with the electronic activity pattern(s) included in the database. The region identification policy may identify the region of the time interval based on which electronic activity pattern included in the database most closely matches the electronic activity pattern of the user. The region identification policy may be used by the system to identify which electronic activity pattern of the database most closely matches the electronic activity pattern of the user based on which electronic activity pattern tracks the electronic activity pattern of the user, which electronic activity pattern has or includes upticks and downticks in electronic activity within the same time frame as the electronic activity pattern of the user, and so forth. The region identification policy may be used by the system to identify the region of the time interval corresponding to the electronic activity pattern which most closely matches the electronic activity pattern of the user.

In some embodiments, the system 200 may be configured to identify values from field-value pairs of the node profile 3004 associated with the user. The system 200 may be configured to identify a value corresponding to a field-value pair associated with a job title for the user, a field-value pair associated with an industry of the enterprise or company in which the user is employed, a location of the enterprise or company, etc. The region identification engine may identify the region associated with the schedule of the user based on the values from the node profile 3004 for the user. In some implementations, the region identification engine may identify the region based on the job title of the user as reflected in the node profile 3004 of the user. For instance, where the job title indicates a higher level position, the user may typically work longer or more consistent hours. On the other hand, where the job title indicates a lower level position, the user may work fewer hours, may be part-time, may work less consistent hours, and so forth. As another example, the location or industry in which the user works may reflect more work from home options, more flexible working hours, and so forth. The region identification engine may leverage the data from the node profile 3004 of the user for modifying, adapting, or otherwise changing the region of the time interval. The region identification engine may expand the region when the user is a higher level position, adjust one or more weights of the temporal distribution (e.g., the weights based on the location of the user) based on the location/industry, and so forth. Each of these implementations may use data from the node profile 3004 of the user to more accurately select or modify the region of the time interval corresponding to the schedule of the user.

The region identification engine may be configured to store an association between the node profile 3004 of the user and the identified region of the time interval corresponding to the schedule of the user. The region identification engine may store the association in a database, memory, server, or other data structure. In some embodiments, the identified region may be static (e.g., once the region is identified, the region remains the same). In some embodiments, the identified region may be dynamic (e.g., the region may be changed over time). In some embodiments, the region identification engine may update the region at various points in time. The region identification engine may update the region on a schedule (e.g., once every six months, once every year, etc.). The region identification engine may update the region upon occurrence of a condition which triggers the update. The condition may be a job change, a promotion, and so forth. The system 200 may identify such conditions based on changes in confidence score of values corresponding to fields of the node profile 3004, as described above in Section 1(D) above.

In some embodiments, the system 200 may leverage the region associated with the node profile 3004 of the user for identifying an event condition. In some embodiments, the system 200 may include an event condition detection engine. The event condition detection engine may be or include any script, file, program, application, set of instructions, or computer-executable code that is configured to determine, identify, or otherwise detect an event condition based on a comparison of temporal distributions to an electronic activity pattern of a user. The event condition may be or correspond to vacation, a sick day, leave (e.g., maternity, paternity, adoption, etc.), and so forth. Following generation of the electronic activity pattern by the pattern determination engine, the event condition detection engine may leverage the electronic activity pattern to detect event conditions. In some embodiments, the event condition detection engine may include, maintain, or otherwise access an event condition detection policy. The event condition detection policy may be or include a rule-based policy, a probabilistic model, etc. The event condition detection engine may be configured to apply the event condition detection policy to subsequent temporal distributions to determine whether the temporal distribution satisfies the policy for detecting a corresponding event condition.

In some embodiments, the event condition detection policy may include a machine learning model. The machine learning model may be a model which is generated, produced, or otherwise trained to classify temporal distributions of electronic activity. The machine learning model may be trained based on data sets, samples, or other training examples of electronic activity patterns or temporal distributions (or series of temporal distributions) and corresponding labels associated with respective event conditions. For instance, the machine learning model may be trained with a set of training examples corresponding to a first event condition (e.g., a sick day) using a plurality sample temporal distributions corresponding to the first event condition (e.g., the sick day). Each sample temporal distribution may be tagged or labeled with the respective event condition (e.g., a sick day label). Continuing this example, where a person is taking a sick day, the overall number of electronic activity engaged in by the person may decrease for the sick day. The machine learning model may be trained with temporal distributions corresponding to the sick day such that, where a temporal distribution corresponding to a sick day is provided as an input to the machine learning model, the machine learning model is configured to generate a label (or a confidence score corresponding to the label) identifying the temporal distribution as a sick day. As another example, the machine learning model may be trained with a set of training examples corresponding to a second event condition (e.g., a vacation) using a plurality sample temporal distributions in series corresponding to the second event condition (e.g., the vacation). Each sample temporal distribution may be tagged or labeled with the respective event condition (e.g., a vacation label). Continuing this example, where a person is taking a vacation, the overall number of electronic activity engaged in by the person may decrease to below a threshold for a series of days in a row (e.g., a week, for instance). The machine learning model may be trained with the series of temporal distributions corresponding to the vacation such that, where a series of temporal distributions of a user corresponding to a vacation is provided as an input to the machine learning model, the machine learning model is configured to generate a label (or a confidence score corresponding to the label) identifying each of the temporal distributions as vacation time.

As described in greater detail above, the temporal distribution generation engine may be configured to generate various temporal distributions of electronic activity for various time intervals.

In some embodiments, the temporal distribution generation engine may generate a temporal distribution for a set of electronic activities engaged in by the user after generation of the electronic activity pattern (e.g., at a subsequent point of time). The temporal distribution generation engine may generate the temporal distribution for each electronic activity engaged in by the user during a subsequent time interval (referred to herein as a subsequent temporal distribution). The event condition detection engine may be configured to apply the event condition detection policy to the subsequent temporal distribution to determine whether the subsequent temporal distribution satisfies the event condition detection policy for identifying an event condition.

In some embodiments, the event condition detection engine may be configured to identify, determine, or otherwise detect the event condition corresponding to a temporal distribution based on one or more characteristics of the subsequent temporal distribution satisfying a corresponding characteristic associated with the respective event condition. The event condition detection engine may maintain, include, or otherwise access a plurality of characteristics associated respective labels for corresponding event conditions. For instance, a characteristic associated with an event condition for a sick day may indicate a drop in electronic activity engaged in by the user for the time interval below a standard deviation (or other threshold) from an average electronic activity engaged in by the user. The event condition detection engine may access a characteristic associated with the event condition (e.g., sick day). The event condition detection engine may determine whether the subsequent temporal distribution of the user satisfies the characteristic (e.g., the average electronic activities engaged in by the user in the subsequent temporal distribution is outside of a standard deviation from the average electronic activity engaged in by the user). The event condition detection engine may detect the event condition (e.g., the sick day) based on the temporal distribution satisfying the characteristic. As another example, where the user takes a vacation day, a temporal distribution for a business day may include less than threshold number of electronic activities engaged in by the user (e.g., zero or close to zero electronic activities). The event condition detection engine may store, include, maintain, or otherwise access a characteristic associated with the condition corresponding to a vacation day (e.g., average electronic activities or number of electronic activities engaged in by a given user falling below a threshold). The event condition detection engine may be configured to detect an event condition corresponding to a vacation day based on the subsequent temporal distribution satisfying the corresponding characteristic associated with the vacation day event condition.

The event condition detection engine may be configured to store an association between the node profile 3004 of the user and the event condition. The event condition detection engine may be configured to store the association between the node profile 3004 and event condition to more accurately reflect the user's schedule. The event condition detection engine may be configured to store the association between the node profile 3004 and event condition and in association with the region of the time interval corresponding to the user's work schedule. In some embodiments, the system 200 may be configured to generate a report corresponding to a schedule of the user. The report may be a schedule of the user across the time period. For instance, the system 200 may be configured to generate the report to reflect each time interval in which the user worked, and reflect the event conditions. The system 200 may be configured to generate a weekly report, a monthly report, etc. The system 200 may be configured to communicate the report to the user, to a manager, leader, or other employee managing the user, and so forth.

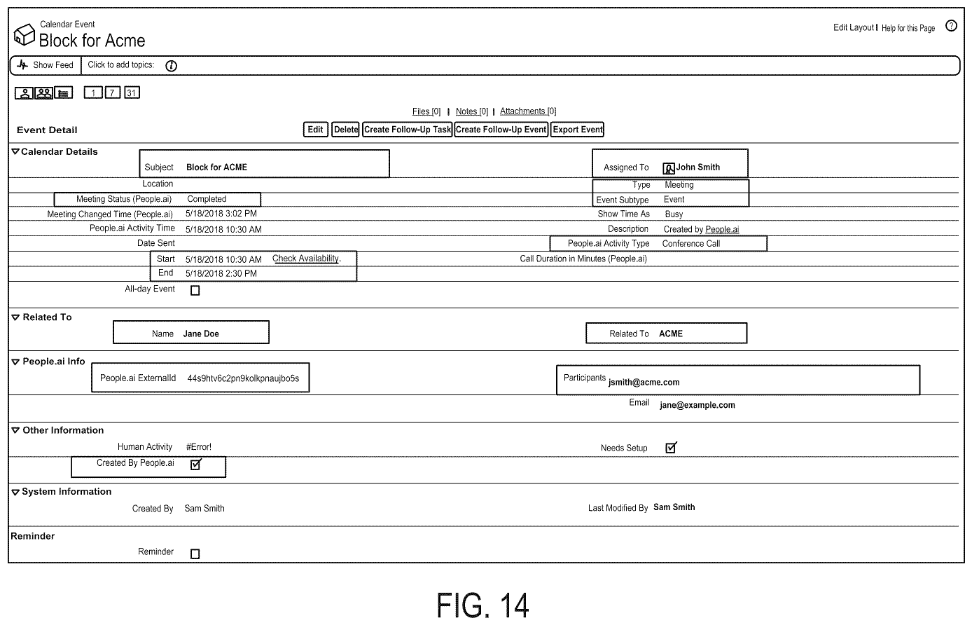

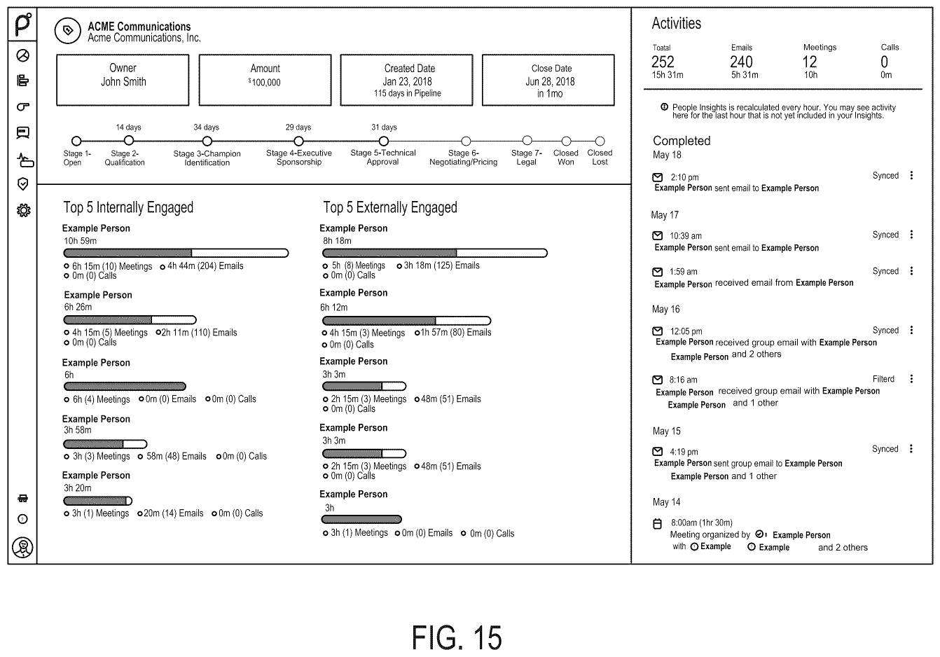

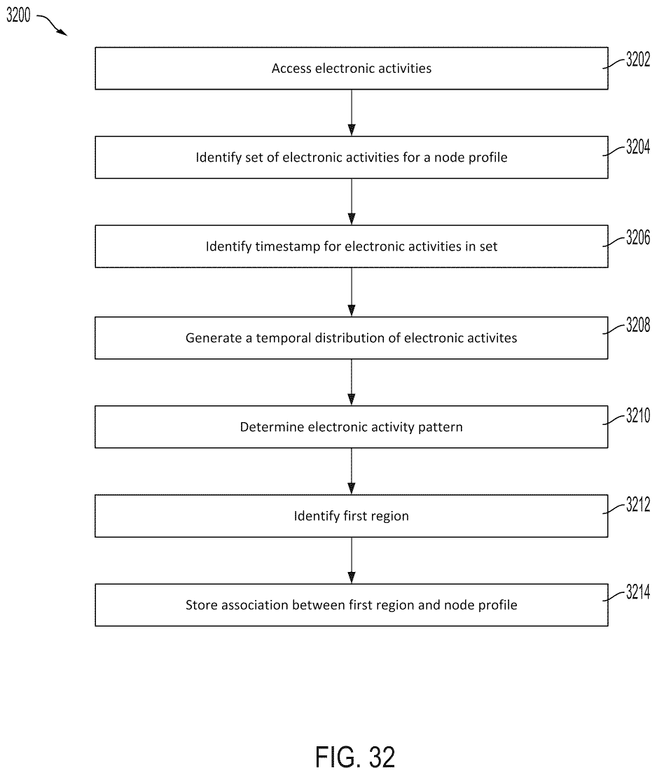

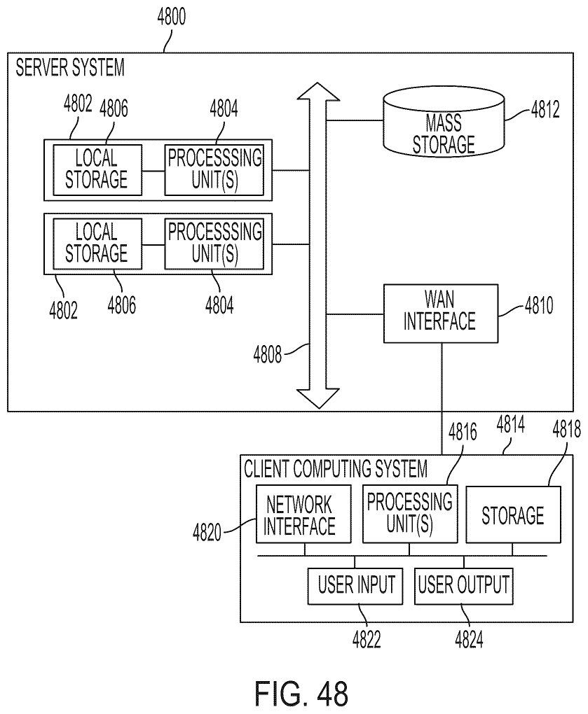

Referring now to FIG. 32, illustrated is a flow diagram of a method 3200 of inferring a work schedule pattern using electronic activities of node profiles. The method 3200 can be implemented or performed using any of the components described above in conjunction with FIGS. 1-29 (e.g., the node graph generation system 200) or the server system 4800 detailed below in conjunction with FIG. 48. The method 3200 may include accessing electronic activities (3202). The method 3200 may include identifying a set of electronic activities for a node profile (3204). The method 3200 may include identifying a timestamp for electronic activities in the set (3206). The method 3200 may include generating a temporal distribution of electronic activities (3208). The method 3200 may include determining an electronic activity pattern (3210). The method 3200 may include identifying a first region (3212). The method 3200 may include storing an association between the first region and the node profile (3214).

The method 3200 may include accessing electronic activities (3202). In some embodiments, the system 200 may access a plurality of electronic activities transmitted or received by a plurality of participants corresponding to one or more respective electronic accounts associated with a plurality of data source providers. The system 200 may access the electronic activities as they are generated, sent, received, exchanged, or otherwise engaged in by the participants. In some embodiments, the system 200 may access the electronic activities when they are ingested into the system 200 from the data source provider(s). Each of the electronic activities may include metadata corresponding to, identifying, or otherwise used for identifying participants of the electronic activity. For instance, the metadata may include a sender/initiator, recipient, other participants, etc. of the electronic activity. Each of the electronic activities may include a timestamp. The timestamp may indicate the time in which the electronic activity was generated, sent, received, or otherwise engaged in by the participant(s).

The method 3200 may include identifying a set of electronic activities for a node profile (3204). In some embodiments, the system 200 may identify a set of electronic activities for a node profile engaged in by a participant of the plurality of participants linked to the node profile within a time period. Each node profile may include, for instance, a value for a field-value pair corresponding to an email address, a value for a field-value pair corresponding to a phone number, etc. The system 200 may identify the set of electronic activities for the node profile based on which electronic activities include a matching value from the metadata in the node profile. As described above, the metadata may include a sender, recipient(s), etc. The system 200 may cross-reference the values from the metadata with data from the node profiles to identify the set of electronic activities for the node profile. The system 200 may identify the set of electronic activities within a time period. The time period may be, for instance, a 32-day window, a 90-day window, a 180-day window, and so forth.

In some embodiments, the system 200 may determine a type of each electronic activity engaged in by a participant linked to the node profile within a time period. The system 200 may select the electronic activities for the set having a specific type. The system 200 may determine the type of each electronic activity engaged in by determining which value for the field-value pair of the node profile was used for associating the electronic activity to the node profile. The system may correlate specific fields of a node profile with specific types of electronic activity (e.g., field corresponding to email address with an email electronic activity type, field corresponding to work phone number with a phone call electronic activity type, and so forth). The system 200 may identify the electronic activity type for each electronic activity engaged in by the user within the time period. The system 200 may include, in the set of electronic activity, a subset of type(s) of electronic activity. For instance, in some embodiments, the system 200 may limit the set to include only emails and phone calls, as such electronic activity may be more typical of a schedule for a user.

The method 3200 may include identifying a timestamp for electronic activities in the set (3206). In some embodiments, the system 200 may identify a timestamp at which each electronic activity was sent or received in the set of electronic activities (e.g., identified at 3204). The system 200 may parse the metadata of each electronic activity in the set to identify the timestamp for each electronic activity in the set. The timestamp may be a time in which the electronic activity was sent, received, or otherwise engaged in by the user. The system 200 may sort each of the electronic activities by their respective timestamps. Hence, each electronic activity may be sorted or otherwise arranged in chronological order.

The method 3200 may include generating a temporal distribution of electronic activities (3208). In some embodiments, the system 200 may generate a temporal distribution of electronic activity for each time interval within the time period based on respective timestamps of each electronic activity of the set of electronic activities within the time interval. The system 200 may separate each electronic activity within the set into subsets. Each time interval may correspond to, for instance, one day of the time period, one week of the time period, etc. The system 200 may maintain, for each time period, a set of time intervals. The system 200 may generate the temporal distribution responsive to identifying the electronic activities in the set (e.g., at 3206). The system 200 may generate the temporal distribution for each time interval of the time period. The system 200 may generate the temporal distribution in real-time. The system 200 may generate the temporal distribution at the end of each time interval. The system may generate the temporal distribution of electronic activities by compiling, for each time frame (e.g., 15 minute, half hour, hour, etc.) within the time interval, the number of electronic activities engaged in during the respective time frame. Hence, the temporal distribution may reflect the number of electronic activities engaged in by the user across the time interval.

The method 3200 may include determining an electronic activity pattern (3210). In some embodiments, the system 200 may determine an electronic activity pattern based on the temporal distribution of electronic activity for each time interval within the time period. The system 200 may determine the electronic activity pattern following generation of each temporal distribution (e.g., at each iteration of 3208). The system 200 may determine the electronic activity pattern following generation of a temporal distribution for each time interval within the time period. The system 200 may generate the electronic activity pattern based on each of the temporal distributions (e.g., generated at 3208) that fall within the time period. The system 200 may determine the electronic activity pattern by applying a pattern determination policy to each of the temporal distributions within the time period. The pattern determination policy may be a rule-based policy, a probabilistic model, etc. The pattern determination engine may be configured to apply the pattern determination policy to each of the temporal distributions within the time period for generating the electronic activity pattern for the user. The pattern determination policy may compute an average, mean, etc. of electronic activity for each time frame within a respective time interval in the time period. In some embodiments, the pattern determination policy may filter one or more data outliers in the temporal distribution (e.g., a number of electronic activities exceeding a standard deviation from the average or mean of electronic activity within a respective time frame). In some embodiments, the pattern determination policy may be used by the pattern determination engine to filter electronic activities within a respective time frame having a mean or average less than one electronic activity.

The method 3200 may include identifying a first region (3212). In some embodiments, the system 200 may identify a first region of the electronic activity pattern satisfying a region identification policy. The first region may be a portion of the electronic activity pattern corresponding to a time range within the time interval that is associated with a work schedule for the user. The system 200 may apply the region identification policy to the electronic activity pattern for identifying the first region. The region identification policy may be or include a probabilistic model, a comparator, etc.

In some embodiments, the system 200 may identify the first region by applying a probabilistic model to the electronic activity pattern. The probabilistic model may be trained using electronic activity patterns and corresponding ranges associated with work schedules. The work schedules may be tagged, flagged, validated, or otherwise confirmed as accurate. The probabilistic model may be trained by providing, to a machine learning algorithm, a group of electronic activity patterns and corresponding work schedules. As more electronic activity patterns and corresponding work schedules are provided to the machine learning algorithm, the machine learning algorithm may refine the probabilistic algorithm to increase the likelihood that the probabilistic model correctly identifies the range associated with a corresponding electronic activity pattern.

In some embodiments, the system 200 may identify the first region by comparing the determined electronic activity pattern (e.g., at 3210) to a plurality of stored electronic activity patterns corresponding to respective regions of the electronic activity pattern. The system 200 may store, maintain, include, or otherwise access a plurality of electronic activity pattern. The system 200 may compare the determined electronic activity pattern with the accessed electronic activity patterns to determine which accessed electronic activity pattern most closely matches the determined electronic activity pattern. The system 200 may identify the first region based on which of the plurality of electronic activity pattern most closely matches the determined electronic activity pattern. Each accessed electronic activity pattern may include or be associated with a corresponding range. The system may identify the first region as the region corresponding to the accessed electronic activity pattern which most closely matches the determined electronic activity pattern. The system may identify which accessed electronic activity pattern most closely matches the determined electronic activity pattern based on the first time corresponding to an uptick in electronic activity and a second time corresponding to a downtick in electronic activity for both respective electronic activity patterns being substantially the same, based on a positive and negative slope of a graphical representation of the respective electronic activity patterns being substantially the same, etc.

In some embodiments, the system 200 may identify one or more values for a field-value pair of the node profile for the user. The system 200 may identify the first region based on the electronic activity pattern and the one or more values of the field-value pair of the node profile. For instance, the system 200 may identify a value corresponding to a field-value pair associated with a job title for the user, a field-value pair associated with an industry of the enterprise or company in which the user is employed, a location of the enterprise or company, etc. The system 200 may identify the region associated with the schedule of the user based on the values from the node profile for the user. In some implementations, the system 200 may identify the region based on the job title of the user as reflected in the node profile 3004 of the user. The system 200 may leverage the data from the node profile of the user for modifying, adapting, or otherwise changing the region of the time interval. The system 200 may expand the region based on the job title value, adjust one or more weights used for generation of the temporal distribution based on the location/industry value(s), and so forth. Each of these implementations may use data from the node profile of the user to more accurately select or modify the region of the time interval corresponding to the schedule of the user.

The method 3200 may include storing an association between the first region and the node profile (3212). In some embodiments, the system 200 may store an association between the first region and the node profile in one or more data structures. The one or more data structures can be maintained by the data processing system 9300 or the system 200. The system 200 store an association between the node profile of the user and the identified region of the time interval corresponding to the schedule of the user. The system 200 may store the association in a database, memory, server, or other data structure. In some embodiments, the identified region may be static (e.g., once the region is identified, the region remains the same). In some embodiments, the identified region may be dynamic (e.g., the region may be changed over time). In some embodiments, the region identification engine may update the region at various points in time. The system 200 may update the region on a schedule (e.g., once every six months, once every year, etc.). The system 200 may update the region upon occurrence of a condition which triggers the update. The condition may be a job change, a promotion, and so forth.

The system 200 may identify such conditions based on changes in confidence score of values corresponding to fields of the node profile.

In some embodiments, the system 200 may detect an event condition. The system 200 may detect an event condition by identifying a second set of electronic activities engaged in by the participant corresponding to the node profile within a second time period (e.g., similar to 3204). The system 200 may generate a temporal distribution of electronic activity for the time interval in the second time period (e.g., similar to 3208). The system 200 may compare the temporal distribution to the determined electronic activity pattern for the user to determine that the temporal distribution satisfies an event condition detection policy for detecting an event condition. The system 200 may store an association between the node profile and the event condition in one or more data structures.

In some embodiments, the event condition detection policy can be used by the system to detect the event condition from a plurality of event conditions based on the determined electronic activity pattern satisfying a corresponding characteristic of a plurality of characteristics associated with a respective event condition. The event condition detection engine may access a plurality of characteristics associated respective labels for corresponding event conditions. For instance, a characteristic associated with an event condition for a sick day may indicate a drop in electronic activity engaged in by the user for the time interval below a standard deviation (or other threshold) from an average electronic activity engaged in by the user. The event condition detection engine may access a characteristic associated with the event condition (e.g., sick day). The event condition detection engine may determine whether the subsequent temporal distribution of the user satisfies the characteristic (e.g., the average electronic activities engaged in by the user in the subsequent temporal distribution is outside of a standard deviation from the average electronic activity engaged in by the user). The event condition detection engine may detect the event condition (e.g., the sick day) based on the temporal distribution satisfying the characteristic. As another example, where the user takes a vacation day, a temporal distribution for a business day may include less than threshold number of electronic activities engaged in by the user (e.g., zero or close to zero electronic activities). The event condition detection engine may access a characteristic associated with the condition corresponding to a vacation day (e.g., average electronic activities or number of electronic activities engaged in by a given user falling below a threshold). The event condition detection engine may detect an event condition corresponding to a vacation day based on the temporal distribution satisfying the corresponding characteristic associated with the vacation day event condition.

In some embodiments, the event condition detection policy includes a machine learning model trained to classify temporal distributions of electronic activity. The system 200 may use the machine learning model to determine that the temporal distribution satisfies the event condition detection policy. In some embodiments, the machine learning model may be trained to identify several event condition types. The machine learning model may use a first set of training examples including temporal distributions corresponding to a first event condition type and a first label identifying the first event condition type. The machine learning model may use a second set of training examples including temporal distributions corresponding to a second event condition type and a second label identifying the first event condition type. The system 200 may train the machine learning model using the first and second set of training examples. Following training, the machine learning model may detect the first and second event conditions based on traits, characteristics, and so forth of the temporal distributions of the user for a time interval. Continuing the examples described above, the machine learning model may be trained to detect event conditions corresponding to sick days and vacation days. For instance, the machine learning model may be trained with a set of training examples corresponding to a first event condition (e.g., a sick day) using a plurality sample temporal distributions corresponding to the first event condition (e.g., the sick day). Each sample temporal distribution may be tagged or labeled with the respective event condition (e.g., a sick day label). Continuing this example, where a person is taking a sick day, the overall number of electronic activity engaged in by the person may decrease for the sick day. The machine learning model may be trained with temporal distributions corresponding to the sick day such that, where a temporal distribution corresponding to a sick day is provided as an input to the machine learning model, the machine learning model is configured to generate a label (or a confidence score corresponding to the label) identifying the temporal distribution as a sick day.

As another example, the machine learning model may be trained with a set of training examples corresponding to a second event condition (e.g., a vacation) using a plurality sample temporal distributions in series corresponding to the second event condition (e.g., the vacation). Each sample temporal distribution may be tagged or labeled with the respective event condition (e.g., a vacation label). Continuing this example, where a person is taking a vacation, the overall number of electronic activity engaged in by the person may decrease to below a threshold for a series of days in a row (e.g., a week, for instance). The machine learning model may be trained with the series of temporal distributions corresponding to the vacation such that, where a series of temporal distributions of a user corresponding to a vacation is provided as an input to the machine learning model, the machine learning model is configured to generate a label (or a confidence score corresponding to the label) identifying each of the temporal distributions as vacation time.

The event condition detection engine may store an association between the node profile of the user and the event condition. The event condition detection engine may store the association between the node profile and event condition to more accurately reflect the user's schedule. The event condition detection engine may store the association between the node profile 3004 and event condition and in association with the region of the time interval corresponding to the user's work schedule.

In some embodiments, the system 200 may generate a report corresponding to a schedule of the user. The report may be a schedule of the user across the time period. For instance, the system 200 may generate the report to reflect each time interval in which the user worked, and reflect the event conditions. The system 200 may be configured to generate a weekly report, a monthly report, etc. The system 200 may be configured to communicate the report to the user, to a manager, leader, or other employee managing the user, and so forth.

### 22. Classifying Phone Numbers Based on Node Profile Data

Enterprises and other entities can spend significant amounts of resources to maintain and update one or more systems of records. Examples of systems of records can include customer relationship management (CRM) systems, enterprise resource planning (ERP) systems, document management systems, applicant tracking systems, among others. Typically, these systems of records are manually updated, which can result in multiple issues. For example, the systems of record may include an outdated, incorrect, or incomplete list of phone numbers associated with a respective customer. For example, although a customer has indicated through an email that he can be reached directly via a new business direct phone number, a system of record may include only a business general phone number for that customer, for which there is a queue of callers waiting at any given time. Thus, attempting to contact the customer by using the system of record may result in unnecessary delay. Furthermore, updating the system of record requires using computer and network resources, such as network BW, CPUs, RAM, and storage, to perform a manual search of the email, fetch the email, and transfer the new phone number from the email to the system of record. The extra steps needed may result in unnecessary resource usage and a reduction in available resources for more critical enterprise tasks. Thus, there is a technical problem of maintaining up-to-date phone numbers and other contact information in a system of record without overburdening the computer and network resources associated with an enterprise.

This disclosure aims to address these technical challenges with improved systems and methods for classifying phone numbers based on node profile data. The present disclosure offers a technical solution to the technical problem of identifying phone numbers in electronic activities and maintaining phone numbers in the system of record without overusing computer and network resources. The present disclosure includes systems and methods to parse an electronic activity to identify an electronic activity phone number. The systems and methods described herein can use a data structure to determine a type of phone number to which the electronic activity phone number corresponds. One or more node profiles corresponding to one or more participants of the electronic activity can be identified. The identified node profiles can be updated based on the type of phone number. The updated node profiles can also be used to update a system of record.

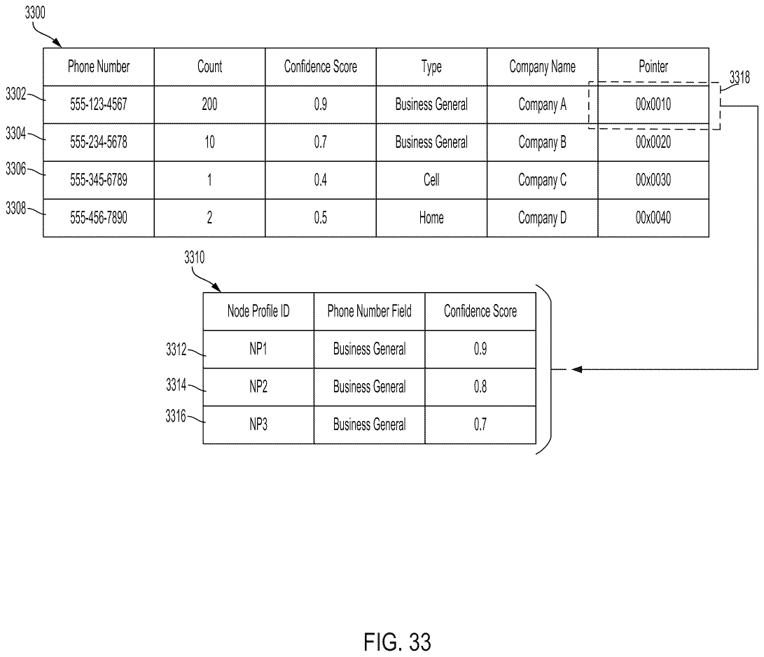

FIG. 33 illustrates an example representation of a data structure 3300 configured for classifying phone numbers based on node profile data. The data structure 3300 may include one or more entries such as an entry 3302, an entry 3304, an entry 3306, and an entry 3308. Each of the entries includes a phone number, a count of a number of node profiles having a node profile phone number matching the phone number, a phone number type associated with the phone number, a company associated with the phone number, and a confidence score indicating a confidence that the phone number is being associated with a correct phone number type in the data structure 3300. For example, a confidence score may indicate a confidence that a phone number value such as the value “5555-123-4567” for the entry 3302 is associated with a correct phone number type such as “business general.” The phone number types may include home phone number, personal cell phone number, business general phone number, and business direct phone number. For example, a business general phone number may be a phone number associated with a business but not necessarily associated with a particular employee of the business. In contrast, a business direct phone number may be a business phone number that dials directly to a phone of a particular employee of the business. It should be understood that these phone number types are illustrative only, and that in some embodiments, the data structure 3300 may also identify different types of phone numbers not depicted in FIG. 33.

Each entry in the data structure 3300 may be linked to a respective second data structure 3310 including one or more second entries. For example, the entry 3302 may be linked to the second data structure 3310 including a second entry 3312, a second entry 3314, and a second entry 3316. Each second entry may include information corresponding to or retrieved from portions of node profiles having a node profile phone number value matching the phone number of the respective entry in the data structure 3300. Each second entry may include an identifier of a node profile, a node profile phone number field to which the node profile phone number value is associated, and a second confidence score indicating a confidence that the node profile phone number field is associated with a correct node profile phone number value. For example, the second confidence score may indicate the confidence that a “business general” node profile phone number field has a correct node profile phone number value of “555-123-4567.” In some embodiments, the second confidence scores of the second data structure 3310 can be referred to as individual confidence scores, because each one is associated with an individual node profile. Similarly, the confidence scores of the data structure 3300 may be referred to in some embodiments as aggregate confidence scores, because they may depend on or may represent an aggregation of more than one node profile (e.g., each node profile having an entry in the second data structure 3310).

In some embodiments, each entry in the data structure 3300 may include a pointer, such as the pointer 3318, that points to the respective entry of the second data structure 3310. Each pointer may be or may include an index or a memory address at which a corresponding portion of the respective second data structure 3310 is stored. In some embodiments, the memory address may be a starting memory address for the respective second data structure 3310. The pointer 3318 may include a memory address at which one or more of a set of second pointers are stored. In some embodiments, the pointer 3318 includes a hash of an identifier of the respective second data structure 3310.

Each second entry in the second data structure 3310 may include a second pointer including a memory address at which a portion of a respective node profile is stored. The memory address may be a starting memory address for the respective node profile. The second pointer may be a hash of the identifier of the respective node profile. In some embodiments, responsive to a node profile being updated, the second entry pointing to the node profile may be updated.

In some embodiments, the count of each entry in the data structure 3300 may be equal to a number of second entries in the respective data structure 3310. The confidence score of each entry in the data structure 3300 may be computed based on the node profile phone number field-value pairs and the second confidence scores in the respective second data structure 3310. For example, the confidence score of each entry in the data structure 3300 may be an average of at least a subset of the second confidence scores in the respective second data structure 3310. The subset of the second confidence scores may include the second confidence scores that correspond to a node profile phone number field matching the phone number type of the respective entry in the data structure 3300.

In some embodiments, a confidence score can be computed for each of the phone number types in the data structure 3300. The phone number type of each entry in the data structure 3300 may be determined as a phone number type having the highest confidence score for that particular phone number in the second data structure 3310 among all phone number types. In some embodiments, responsive a second data structure 3310 being updated, the confidence scores of each of the phone number types of the respective entry of the data structure 3300 may be re-computed. The second data structure 3310 may be updated by changing a portion of a second entry or adding a new second entry. Responsive to determining that a different phone number type has the highest confidence score based on the re-computation, the phone number type of the respective entry in the data structure 3300 may be updated.

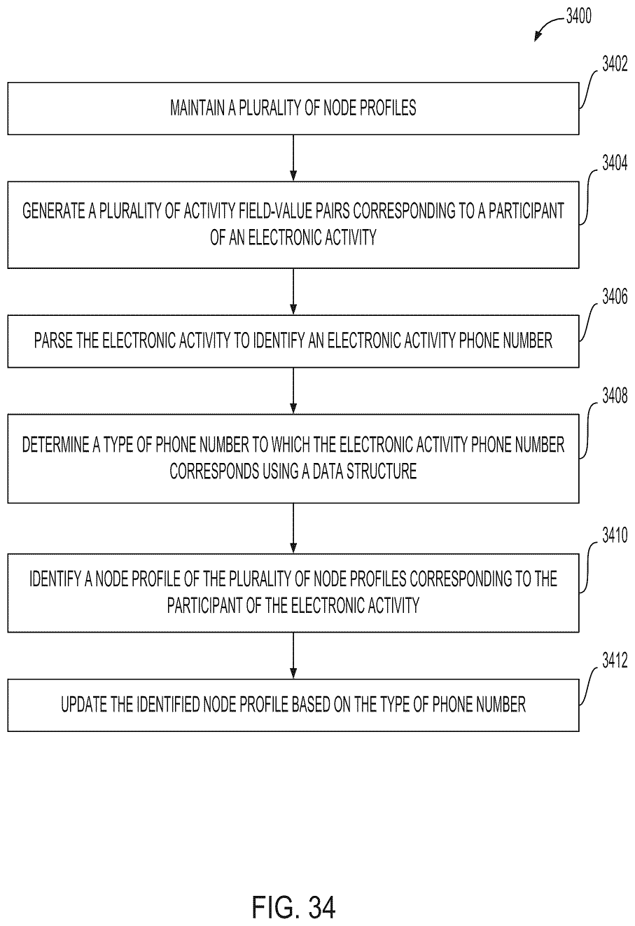

FIG. 34 illustrates a method 3400 for classifying phone numbers based on node profile data, in accordance with one or more embodiments. The operations of method 3400 presented below are intended to be illustrative. In some embodiments, method 3400 may be accomplished with one or more additional operations not described, and/or without one or more of the operations discussed. Additionally, the order in which the operations of method 3400 are illustrated in FIG. 34 and described below is not intended to be limiting.

In some embodiments, method 3400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 3400 in response to instructions stored electronically on an electronic storage medium. The one or more processing devices may include one or more devices configured through hardware, firmware, and/or software to be specifically designed for execution of one or more of the operations of method 3400. In some embodiments, the functionality described in connection with the method 3400 can be performed by a system such as the node graph generation system 200 shown in FIG. 4, as well as its various components. FIG. 34 is therefore described below with reference to certain components of the node graph generation system 200 of FIG. 4.

The method 3400 may include maintaining a plurality of node profiles (BLOCK 3402). In some embodiments, the node profile manager 220 is configured to maintain a plurality of node profiles. Each node profile may include a plurality of fields. Each field may include one or more value data structures. Each value data structure may include a node field value and one or more entries corresponding to respective one or more data points that support the node field value of the value data structure. One or more fields may include a phone number field. One or more corresponding value structures may identify a phone number corresponding to the node profile.

The method 3400 may include generating a plurality of activity field-value pairs from an electronic activity (BLOCK 3404). In some embodiments, the ingestor 205 may be configured to ingest the electronic activity. In some embodiments, the electronic activity parser 210 can be configured to generate the activity field-value pairs from the ingested electronic activity. The activity field-value pairs may include field-value pairs for a first name, a last name, a company name, and the like. Each of the activity field-value pairs may correspond to a participant of the electronic activity.

The method 3400 may include parsing the electronic activity to identify a sequence corresponding to an electronic activity phone number (BLOCK 3406). The electronic activity parser 210 may be configured to parse the electronic activity. The sequence may include a string including a predetermined number of numerical characters satisfying one or more predetermined regular expression (regex) patterns. The sequence may satisfy the regex pattern by having a first predetermined number of numerical characters, followed by zero or more spacer characters which may include a first hyphen, space, or the like, followed by a second predetermined number of characters, followed by zero or more second spacer characters such as a second hyphen, space, or the like, followed by a third predetermined number of characters. In some embodiments, greater than or fewer than three sets of predetermined number of numerical characters, and greater than or fewer than two hyphens, spaces, or the like, may satisfy the regex pattern. For example, the regex pattern may also include at least a fourth set of predetermined numerical characters, which in some embodiments may correspond to an extension associated with the phone number.

In some embodiments, the method 3400 further includes accessing a plurality of electronic activities and generating the node profiles based on information included in the plurality of electronic activities. The plurality of electronic activities may be ingested or accessed to generate a plurality of activity field-value pairs. The plurality of electronic activities may be parsed to identify one or more sequences corresponding to a respective one or more electronic activity phone numbers.

The method 3400 may include using a data structure, such as the data structure 3300 described above in connection with FIG. 33, to determine a phone number type to which the electronic activity phone number corresponds (BLOCK 3408). In some embodiments, the data structure may include a plurality of phone numbers. Each phone number may be stored in the data structure with a corresponding field identifying a phone number type. In some embodiments, the phone number type may be determined based on matching at least a portion of the electronic activity phone number to a corresponding portion of the phone number in the data structure. In some embodiments, the phone number type is determined by matching the area code or the last four digits of the electronic activity phone number to the data structure phone number. Matching at least a portion of the two phone numbers may include masking a second portion of each of the two phone numbers with “wildcard” characters, which may also be referred to herein as “Xs.” For example, if comparing area codes, masking the last seven digits of a phone number with an area code 555 could result in the phone number being shown as 555-XXX-XXXX.

In some embodiments, the phone number type may be one of a home phone number, a cell phone number, a group entity general number, or a group entity direct number. A group entity general number (also referred to herein as a business general phone number) may provide access to an assistant, administrator, receptionist, or automated directory that may transfer or forward a call to an intended recipient. The group entity direct number (also referred to herein as a business direct phone number) may provide direct access to the intended recipient.

In some embodiments, the data structure may be generated to include a count for each phone number corresponding to a number of the node profiles that include a field-value pair having a value that matches the phone number. In some embodiments, a relatively large count for a phone number in the data structure may indicate a relatively higher likelihood that the phone number type associated with the phone number is a group entity (e.g., general or direct) phone number. In some embodiments, a relatively smaller count for a phone number in the data structure may indicate a higher likelihood that the phone number type is a home phone number or cell phone number.

In some embodiments, the data structure may be generated to include an aggregate confidence score. The aggregate confidence score may indicate a confidence that the phone number corresponding to the aggregate confidence score has a correct type value in the data structure. In some embodiments, the aggregate confidence score may be based on respective individual confidence scores (e.g., the confidence scores of the second data structure 3310 in FIG. 33). Each individual confidence score may correspond to a particular node profile phone number value that matches the phone number in the data structure. In some embodiments, the node profile phone number value may correspond to a value of a node profile number field that matches the phone number type in the data structure. Each respective individual confidence score may indicate a confidence that the respective node profile phone number field is associated with a correct respective node profile phone number value. The individual confidence scores may be stored in a second data structure, such as the second data structure 3310, corresponding to the respective phone number of the data structure.

In some embodiments, multiple temporary aggregate confidence scores are determined, wherein each temporary aggregate confidence score is associated with one of the node profile phone number field types. Determining a temporary aggregate confidence scores may be based on a subset of the individual confidence scores. For example, the temporary confidence score may be an average of the subset of the individual confidence scores. Each of the subset of the individual confidence scores may be for a node profile phone number value that corresponds to a node profile phone number field to which the temporary aggregate confidence score is being associated. Determining the aggregate confidence score may include determining the aggregate confidence score as the highest temporary aggregate confidence score. Determining the phone number type may include selecting the phone number type that matches the node profile phone number field with the highest temporary aggregate confidence score.

In some embodiments, the method 3400 includes generating the data structure which is used to determine the phone number type. Generating the data structure may include searching the node profiles and/or electronic activities for all phone numbers and creating, in the data structure an entry for each phone number associated with one or more of the node profiles and/or electronic activities.

Generating the data structure may include computing the count, the confidence score, the phone number type for each phone number, which is detailed herein. Generating the data structure may include storing the count, the confidence score, and the phone number type in the entry corresponding to the respective phone number. Generating the data structure may include generating new entries for electronic activity phone numbers which do not match any phone numbers in the data structure.

In some embodiments, the data structure may be generated to include, for each phone number included in the data structure, a frequency of the phone number within the structure. The frequency may indicate how frequently an electronic activity is identified as having a phone number matching with the phone number within the structure. In some embodiments, the data structure may include a full-match frequency and a partial-match frequency. The partial-match frequency may indicate how frequently an electronic activity is identified as having a phone number partially matching with the phone number within the structure. The full-match frequency may indicate how frequently an electronic activity is identified as having a phone number fully matching with the phone number within the structure.

In some embodiments, the data structure may be generated to include, for a first set of phone numbers in the data structure, a value corresponding to a group entity name. The group entity name may include an identifier of an enterprise that smaller entities are members of. The first set of phone numbers may include phone numbers for the enterprise and/or for the smaller entities. In some embodiments, the data structure may include a value corresponding to a confidence score of the group entity name.

In some embodiments, the method 3400 may further include determining that a phone number type of the electronic activity is a group entity phone number based on a match. Determining the phone number type based on the match may include accessing a publicly available database (e.g. on a website) to determine a first phone number of a group entity associated with a group node profile that is linked to a plurality of member node profiles. Determining that the phone number type is based on the match may include comparing the electronic activity phone number to the first phone number. Responsive to determining that the phone number type matches, the method 3400 may further include locating, in the data structure, a second phone number that matches the electronic activity phone number and updating a confidence score to which the second phone number corresponds. Updating the confidence score may also include increasing the confidence score if a phone number type of the second phone number matches the group entity phone number and decreasing the confidence score if the phone number type of the second phone number does not match the group entity phone number.

Determining a type of phone number to which the electronic activity phone number corresponds may include using a probabilistic model to determine a probability score indicating a likelihood that the electronic activity phone number is the type of phone number. The probability score may be based on the count in the data structure to which the electronic activity phone number corresponds or the confidence score in the data structure to which the electronic activity phone number corresponds. The probability score may include the confidence score or may be proportional to the confidence score.

The probability score may be based on parsing an electronic activity. For example, a first entry may include a direct business phone number with a low confidence score and/or probability score. An electronic activity is parsed and the same direct business phone number appears after a string that says “my direct business phone number is.” Based on detecting the content and proximity of the string to the direct business phone number, the probability of the confidence score and/or probability score may increase.

The probability score may be based on a relationship between two node profiles corresponding to the electronic activity. For example, if the two node profiles are identified as the sender and the recipient of the electronic activity but there is a weak relationship between the two node profiles, then the probability score for the electronic activity phone number may be low.

Determining the type of phone number using the probabilistic model may include determining the probability score based on the frequency of the phone number. For example, if the phone number is frequently identified (e.g., appears as a value of a phone number field in many node profiles), the probability score may be relatively high. If the phone number is infrequently identified, the probability score may be relatively low. In some embodiments, the probabilistic model may be based on other information as well. For example, the probabilistic model may use or otherwise account for information extracted directly from the electronic activity in which the phone number was identified. In some embodiments, the electronic activity parser 210 can be configured to parse the electronic activity for other information, such as information relating to the content of the electronic activity. Such information can provide additional context that may impact the results of the probabilistic model. For example, natural language processing may be used to identify language that refers to the phone number and may provide useful context for accurately classifying the phone number type. In some embodiments, for example, if the phrase “home phone number” immediately precedes or is otherwise located near the string corresponding to the phone number extracted from the electronic activity, the probabilistic model may determine a relatively high degree of probability that the phone number is a home phone number.

The method 3400 may further include classifying the electronic activity as a personal phone number responsive to determining that a location corresponding to a prefix of the electronic activity phone number matches a portion of a residential address of the participant. For example, the prefix may indicate that the location is San Diego and a portion of the residential address of the participant may include San Diego. Likewise, the electronic activity may be classified as a cell phone number responsive to determining that a location corresponding to a prefix of the electronic activity phone number does not match a portion of a residential address of the participant. The method may further include determining the location corresponding to the prefix. Determining the location corresponding to the prefix may include accessing a publicly available database and looking up, in the publicly available database, the location at an index corresponding to the prefix. The method may further include determining the residential address of the participant. Determining the residential address of the participant may include extracting the residential address from the electronic activity. Determining the residential address of the participant may include reading a value corresponding to a residential address field of the node profile of the participant.

The method 3400 may include identifying a node profile of the plurality of node profiles corresponding to the participant of the electronic activity to which the electronic activity phone number corresponds (BLOCK 3410). For example, the node profile may correspond to a sender or one or more recipients of the electronic activity. The node profile manager 220 may be configured to identify a node profile corresponding to the participant of the electronic activity. In some embodiments, identifying a node profile of the plurality of node profiles corresponding to the participant of the electronic activity includes determining that the extracted field-value pairs of the electronic activity and the one or more values of the fields of the at least one node profile satisfy a node profile matching policy. In some embodiments, more than one node profile may be identified as corresponding to the participant of the electronic activity.

In some embodiments, the matching policies for the matching of the electronic activity to one or more node profiles is based on tags associated with the electronic activity. For example, satisfying the node profile matching policy can include determining a relationship between two or more node profiles based on the one or more values of the fields of the two or more node profiles. Satisfying the node profile matching policy can include assigning one or more tags to the electronic activity based on the relationship between the two or more node profiles. In some embodiments, satisfying the node profile matching policy includes determining that two node profiles have a personal (non-professional) relationship based on the electronic activities exchanged between the users associated with the node profiles and apply a “personal” tag to the emails between the users. In some embodiments, satisfying the node profile matching policy includes determining if two nodes have a personal relationship based on commonalities in values in their node profiles, for instance, their home addresses (if they are neighbors), college or school affiliations (alumni/classmates), same last names, other non-professional affiliations, or other signals that may indicate the two node profiles may have a personal relationship.

The method 3400 may include updating the identified node profile based on the phone number type (BLOCK 3412). Updating the identified node profile may include adding the electronic activity phone number as a new value of a field corresponding to the determined phone number type. For example, if the identified node profile listed as a value “555-123-4567” under the field for a home phone number, and the electronic activity phone number was extracted as “555-234-5678”, then the electronic activity phone number “555-2345-678” can be added as a new value of the field for a home phone number. The home field may include both values or may overwrite the previous value. In some embodiments, the field may be a new field. For example, if the identified node profile only included a field for a home phone number and the phone number type was for a cell phone number, then a new field for the cell phone number can be added as a new field and the new value can be added to the new field.

Updating the identified node profile may include updating the identified node profile by adding an extension number as a new value of the field corresponding to the determined phone number type. Adding the extension number may be responsive to parsing the electronic activity to identify the extension number as a number associated with the electronic activity phone number. Parsing the electronic activity to identify the extension number may include identifying a set of numbers in proximity to the electronic activity phone number. In proximity refers herein to being within a predetermined number of characters. In some embodiments, parsing the electronic activity to identify the extension number may include satisfying a regex pattern for extension numbers. An example of such a regex pattern would be the word “extension” or “ext.” followed by a set of numbers.

Updating the identified node profile may include updating a confidence score of an existing field-value pair of a node profile corresponding to the electronic activity phone number. Updating the confidence score may include increasing the confidence score responsive to determining that the electronic activity phone number is the same as the value of the field-value pair and that a phone number type extracted from the electronic activity is the same as the field of the field-value pair. Updating the confidence score may include decreasing the confidence score responsive to determining that one of the electronic activity phone number or the phone number type does not match the value or the field, respectively. Updating the confidence score may include increasing the confidence score responsive to the electronic activity being successful (e.g. a successfully received email). Updating the confidence score may include decreasing the confidence score responsive to the electronic activity being unsuccessful (e.g. a bounce back email).

In some embodiments, the method 3400 further includes determining from content included in the electronic activity, that the phone number type of the electronic activity phone number cannot be determined based on the content included in the electronic activity. For example, an email corresponding to a node profile may not include any numbers or may include numbers that do not satisfy a regex pattern.

### 23. Confirming Meeting Events Using Electronic Activities

The present disclosure relates to systems and methods for confirming meeting events using electronic activities. A meeting event can be a virtual meeting event that requires no participants to physically join the meeting event, an in-person meeting event that requires each participant to physically join the meeting event, or a combination thereof. The meeting event may be scheduled through an electronic activity (e.g., an invitation email or an electronic calendar appointment item) prior to the event actually occurring. A participant can reply to the invitation email or other electronic activity representation of the scheduled event through a reply email (e.g., RSVP), indicating whether the participant will attend the meeting event. In some instances, it may be challenging to determine whether the participant actually attends the meeting event solely according to such a reply email. For example, a participant may initially reply to the invitation email of an event indicating that he or she will attend the meeting, but under certain circumstances, the participant may later decide not to attend the meeting. Thus, the meeting event may be shown with a high attendance rate that may not reflect the actual attendance rate, which may cause some misinterpretation of the meeting event in terms of its business value and/or importance. Accordingly, it may be desirable to provide a system that does not rely only on self-reported, or self-replied information from candidate participants of a scheduled event, and can therefore more accurately track the status (e.g., an attendance rate, an occurrence rate) of a scheduled event as well as the status of each of the expected and actual attendees. The status of an expected or actual attendee can further be used for measuring performance metrics of the expected or actual attendee based on the absence or presence of the attendee from the scheduled event, a status or progress of one or more opportunity record objects, an availability of the expected or actual attendee at a particular time, among others.

According to the embodiments of the systems and methods described herein, a data processing system can automatically identify a number of electronic activities for a candidate participant of a scheduled event. The system can first identify an initial electronic activity (e.g., an invitation email, a replied email to an invitation email) corresponding to the scheduled event, and can identify a number of other electronic activities for a candidate participant of the scheduled event. A candidate participant can be any person who is invited to attend the scheduled event. The system can determine which of such other electronic activities is relevant to the initial electronic activity. The system can also process the relevant electronic activities to determine a probability that the candidate participant actually attended the scheduled event, and can assign an attendance tag to the electronic activity corresponding to the event. The attendance tag can indicate that the candidate participant actually attended the scheduled event. The system may update the attendance tag, indicating a likelihood that the candidate participant actually attended the scheduled event, by continuously or periodically processing one or more additional electronic activities relevant to the initial electronic activity that corresponds to the scheduled event. As such, the disclosed system can provide a technical solution to the above-identified technical problem and thus accurately track, monitor, or otherwise manage the status of a scheduled event using one or more electronic activities relevant to the scheduled event. In this way, the system can maintain the respective up-to-date statuses of a number of scheduled events based on the automatically identified electronic activities for individuals (e.g., candidate participants) that communicate with one another. Various other benefits and advantages of the present technical solution are apparent based on the description that follows.

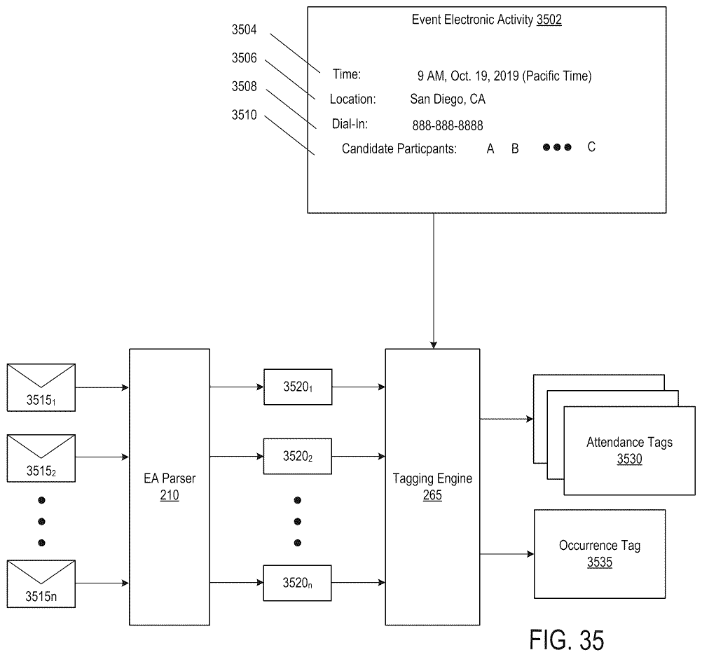

Referring now to FIG. 35, a block diagram is shown of a data processing system that can associate one or more tags with an electronic activity 3502 that corresponds to a scheduled event, based on a number of other electronic activities 35151 to 3515n, in accordance with some embodiments. The electronic activity 3502 corresponding to a scheduled event may also be referred to in this disclosure as an initial electronic activity or as an event electronic activity. For the purposes of describing systems and methods for confirming occurrence and/or attendance of a scheduled event based on one or more other electronic activities, the present disclosure relies in part on FIGS. 3-9, as described above. Thus, the block diagram of FIG. 35 shall be described in conjunction with FIGS. 3-9.

As shown, the system 200, or a component thereof (e.g., the electronic activity ingestor 205), may identify the event electronic activity 3502 as being associated with a scheduled event based on the content or information included in or associated with the event electronic activity 3502. The content can include at least one of a date for a scheduled event, a time for a scheduled event, a location for a scheduled event, a room for a scheduled event, dial-in information, the link to a virtual meeting room, one or more candidate (invited) participants, a topic or document to be discussed during the event, etc. For example, the event electronic activity 3502 may include a time 3504 for a scheduled event (e.g., 9 AM, Oct. 19, 2019, Pacific Time), a location 2086 (e.g., San Diego, California) for the scheduled event, dial-in information 3508 for the scheduled event (e.g., 888-888-8888), and candidate participants 3510 for the scheduled event (e.g., A, B, C, etc.). In some implementations, the system 200 may determine that the event electronic activity 3502 corresponds to a scheduled event based on a format of the event electronic activity 3502. For example, if the event electronic activity 3502 is formatted as an electronic calendar item (e.g., an electronic calendar appointment or invitation), the system 200 may determine that the event electronic activity 3502 corresponds to a scheduled event. In some embodiments, the system can determine or detect an event based on parsing one or more electronic activities that include text that infers the scheduling of a meeting. One or more natural language processing techniques can be used to determine if an electronic activity includes information about scheduling a meeting, a phone call, or other type of event involving two or more participants of the electronic activity.

In response to identifying the event electronic activity 3502, the system 200, or the electronic activity ingestor 205, can access a plurality of additional electronic activities to identify one or more of the additional electronic activities that are associated with each of the candidate participants of the event electronic activity 3502. In some embodiments, the electronic activity ingestor 205 may use an electronic account (e.g., an email account) of each of the candidate participants of the event electronic activity 3502 to identify one or more electronic activities corresponding the event electronic activity 3502. For example, the event electronic activity 3502 can identify the electronic activities 35151 to 3515n from a number of electronic activities, where one or more of the electronic activities 35151 to 3515n is associated with at least one of the candidate participants A, B, or C of the event electronic activity 3502. In response to identifying the electronic activities 35151 to 3515n, the system 200, or a component thereof (e.g., the electronic activity parser 210), may extract one or more features, e.g., feature sets 35201 to 3520n, from each of the electronic activities 35151 to 3515n. Based on the extracted feature sets 3520, the system 200, or a component thereof (e.g., the tagging engine 265), can generate one or more tags (e.g., attendance tag(s) 3530 and occurrence tag(s) 3535) to be associated with the event electronic activity 3502.

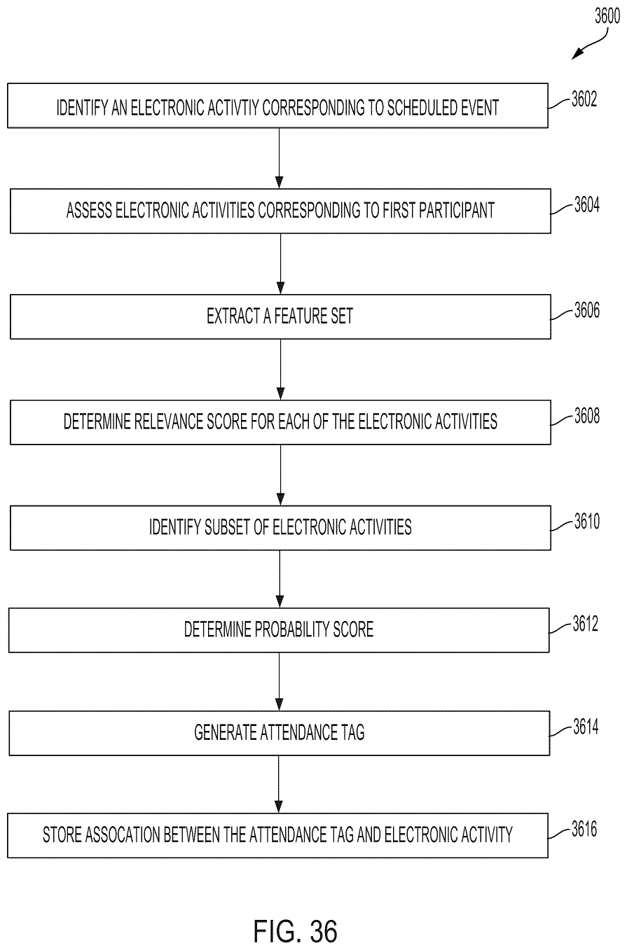

Referring now to FIG. 36, illustrated is a method 3600 for confirming a scheduled event based on one or more electronic activities, in accordance with one or more embodiments. Operations of the method 3600 presented below are intended to be illustrative only. In some embodiments, the method 3600 may be accomplished with one or more additional operations not described, and/or without one or more of the operations discussed. Additionally, the order in which the operations of the method 3600 as illustrated in FIG. 36 and described below is not intended to be limiting.

In some embodiments, the method 3600 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of the method 3600 in response to instructions stored electronically on an electronic storage medium. The one or more processing devices may include one or more devices configured through hardware, firmware, and/or software to be specifically designed for execution of one or more of the operations of the method 3600.

In brief overview, the method 3600 may include identifying an electronic activity corresponding to a scheduled event and identifying a plurality of participant candidates (BLOCK 3602). The method 3600 may include accessing, for a first participant, a plurality of electronic activities corresponding to an electronic account of the first participant (BLOCK 3604). The method 3600 may include extracting, for each of the plurality of electronic activities, a feature set including one or more features based on participants included in the electronic activity or content included in a body of the electronic activity (BLOCK 3606). The method 3600 may include determining, for each of the plurality of electronic activities, a relevancy score between the electronic activity and the scheduled event based on the extracted feature set (BLOCK 3608). The method 3600 may include identifying a subset of the plurality of electronic activities that have respective relevancy scores that satisfy a first threshold value (BLOCK 3610). The method 3600 may include determining a probability score indicating a likelihood that the first participant attended the scheduled event, based on the subset of electronic activities (BLOCK 3612). The method 3600 may include generating, responsive to the probability score satisfying a second threshold value, an attendance tag indicating that the first participant attended the scheduled event (BLOCK 3614). The method 3600 may include storing, in one or more data structures, an association between the attendance tag and the electronic activity corresponding to the scheduled event (BLOCK 3616).

In further detail, the method 3600 may include identifying an electronic activity corresponding to a scheduled event and identifying a plurality of participant candidates (BLOCK 3602). The electronic activity corresponding to the scheduled event may also be referred to in this disclosure as an event electronic activity or an initial electronic activity. In some embodiments, the system 200, or a component thereof (e.g., the electronic activity parser 210), may determine whether an electronic activity corresponds to a scheduled event by parsing the electronic activity. For example, based on information or data parsed from the electronic activity, the electronic activity parser 210 can determine whether the electronic activity includes any calendar-related, schedule-related, and/or meeting-related information, or whether the electronic activity is formatted as a calendar item (e.g., an electronic calendar appointment or invitation). If so, the electronic activity parser 210 can identify the electronic activity as corresponding to a scheduled event; and if not, the electronic activity parser 210 can determine that the electronic activity does not correspond to a scheduled event. With specific reference to FIG. 35, the electronic activity parser 210 may identify the electronic activity 3502 corresponding to a scheduled event based on determining that the electronic activity 3502 includes calendar-related, schedule-related, and/or meeting-related information, e.g., items 3504-2810 of the event electronic activity 3502. In some embodiments, the system can determine or detect an event based on parsing one or more electronic activities that include text that infers the scheduling of a meeting. One or more natural language processing techniques can be used to determine if an electronic activity includes information about scheduling a meeting, a phone call, or other type of event involving two or more participants of the electronic activity.

The method 3600 may include accessing, for a first participant, a plurality of electronic activities corresponding to an electronic account of the first participant (BLOCK 3604). In some embodiments, the system 200, or a component there of such as the electronic activity ingestor 205, can be configured to access or receive a number of electronic activities transmitted or received via electronic accounts associated with one or more data source providers. For example, the electronic accounts may include email accounts. The electronic activity ingestor 205 can access electronic activities via one or more servers hosting or storing the electronic activities to identify the event electronic activity. The servers can store electronic activities transmitted from or received by accounts corresponding to an enterprise. For instance, the servers can be mail servers, phone log servers, calendar servers or any other entity that can store emails, calendar events, phone logs, or other electronic activities of accounts associated with an enterprise, such as a company. The electronic activity ingestor 205, upon receiving the electronic activities, can format metadata or otherwise manage or manipulate the data included in or associated with the electronic activities to facilitate further processing.

Upon identifying an event electronic activity, which can include or can be associated with one or more candidate participants of the scheduled events (e.g., one or more people who were invited to attend the scheduled event), the electronic activity ingestor 205 can access the one or more servers to identify a plurality of electronic activities corresponding to an electronic account of each participant of the one or more participant candidates. In some embodiments, responsive to identifying the event electronic activity by parsing the information of the event electronic activity, the electronic activity parser 210 may communicate or interface with the electronic activity ingestor 205 to access, identify, receive, or otherwise access respective electronic activities of the one or more candidate participants. As shown in the illustrated embodiment of FIG. 35, in response to identifying that the candidate participants of the event electronic activity 352 include candidate participants identified as A, B, and C, and their respective electronic accounts (e.g., email addresses), the electronic activity ingestor 205 can use the email address of each of the participants A, B, and C to identify a set of electronic activities corresponding to the candidate participants. For example, the electronic activity ingestor 205 can use the email address of the candidate participant A (e.g., A@ACME.COM) to identify a set of electronic activities (e.g., 35151-2815n) that include A@ACME.COM as either a recipient or a sender.

The method 3600 may include extracting, for each of the plurality of electronic activities, a feature set including one or more features based on participants included in the electronic activity or content included in a body of the electronic activity (BLOCK 3606). For example, upon identifying the set of electronic activities corresponding to the electronic account of the participant, the electronic activity parser 210 can be configured to parse each of the electronic activities to identify attributes, values, or characteristics of the electronic activity, based on participants included in the electronic activity and/or content included in a body of the electronic activity. The features of the electronic activity can be or can include these attributes, values, or characteristics.

In some embodiments, each participant of each of the electronic activities can be associated with one or more respective values, such as a first name, a last name, an email address, a phone number, an employee identifier, etc. By parsing each of the electronic activities, at least one of the values associated with each of the participants can be identified such that the electronic activity parser 210 can identify the participants (e.g., a sender or a recipient of the electronic activity) of each of the electronic activities. Accordingly, the electronic activity parser 210 can extract a feature corresponding to a sender and/or a recipient of each of the electronic activities. Such an extracted feature corresponding to the sender and/or recipient of each of the electronic activities may be used to determine whether the electronic activity is relevant to the event electronic activity based on whether the sender and/or recipient of the electronic activity is among the candidate participants of the event electronic activity. In some instances, one or more of the electronic activities may be an email that includes a candidate participant of the event electronic activity as one of the recipients but may not be relevant to the scheduled event. Accordingly, the electronic activity parser 210 can extract features corresponding to subjects or topics discussed in the email, which may be used to determine whether the electronic activity is relevant to the scheduled event.

In some embodiments, by parsing each of the electronic activities, the electronic activity parser 210 can extract one or more features corresponding to the content of the body of each of the electronic activities or other content, which can also be used to determine whether the electronic activity is relevant to the event electronic activity. For example, the electronic activity parser 210 can extract from a feature based on a timestamp parsed out of each of the electronic activities. Such a feature corresponding to the timestamp of each of the electronic activities may be used to determine whether the electronic activity is relevant to the event electronic activity based on a time proximity threshold of the timestamp and a scheduled time of the event electronic activity. In another example, the electronic activity parser 210 can extract a feature based on a phone number parsed out of each of the electronic activities, e.g., the body of one of the electronic activities may include a text string such as “call me at 123-222-5899 to discuss the project.” The system 200 can use such a feature corresponding to the phone number to determine whether the electronic activity is relevant to the event electronic activity, for example based on whether the phone number matches a phone number of one of the candidate participants of the event electronic activity.

In yet another example, the electronic activity parser 210 can extract a feature based on a location parsed out of each of the electronic activities, e.g., one or more of the electronic activities may be a confirmation email or receipt email from a travel or transportation company indicating a destination location in the body of the confirmation email. Such a feature corresponding to the location may be used to determine whether the electronic activity is relevant to the event electronic activity based on whether the location is relatively close to a location of the event electronic activity. Alternatively, or additionally, in some embodiments, the electronic activity parser 210 can use a natural language processing (NLP) technique, a keyword search technique, or the like to extract one or more features based on the content of the body of each of the electronic activities. For example, the electronic activity parser 210 can extract, for an electronic activity, a feature corresponding to the event electronic activity based on identifying, via a NLP technique, that a body of the electronic activity includes a word, phrase or other content that relates to the scheduled event and may be used to determine whether a candidate participant actually attended the event. For example, a phrase such as “sorry I couldn't make it” may indicate that the sender of the electronic activity did not attend, even if the sender was a candidate participant. On the other hand, phrases such as “I am looking forward to seeing you in the scheduled event” or “Great seeing you!” may indicate that the sender did attend the scheduled event. The electronic activity parser 210 can extract such features from each of the electronic activities to generate a feature set.

Referring again to FIG. 35, upon identifying the electronic activities 35151-2815n, the electronic activity parser 210 can extract one or more features from each of the electronic activities 35151-2815n, and the features may be represented as feature sets 35201-2820n. Continuing with the above example, upon identifying the electronic activities 35151-2815n that each includes the email address A@ACME.COM as either a recipient or a sender, the electronic activity parser 210 can extract one or more features based on the participants included in the electronic activities 35151-2815n or the content included in the body of the electronic activities 35151-2815n. For example, the electronic activity 35151 may be an email sent from a transportation company to A@ACME.COM to confirm a car ride booked by the candidate participant A. The email may include a body indicating a destination, such as “San Diego, CA.” Accordingly, the electronic activity parser 210 may extract a feature corresponding to the destination, which may be included in one of the feature sets 3520. The system 200 may use such a feature to determine whether the electronic activity 35151 is relevant to the event electronic activity 3502. In another example, the electronic activity 35152 may be an email sending from the email address A@ACME.COM to indicate a doctor appointment. The email may include a body indicating a timestamp of the doctor appointment. Accordingly, the electronic activity parser 210 may extract the feature 35202 corresponding to the timestamp, which may be used to determine whether the electronic activity 35152 is relevant to the event electronic activity 3502. In yet another example, the electronic activity 3515n may be an email sent from the email address A@ACME.COM to an organizer of the scheduled event. Accordingly, the electronic activity parser 210 may extract the feature 3520n corresponding to the recipient of the electronic activity, which may be used to determine whether the electronic activity 3515n is relevant to the event electronic activity 3502. For example, such an electronic activity may be an RSVP relating to the scheduled event. It should be appreciated that the electronic activity parser 210 can extract more than one feature from each electronic activity to form a feature set 3520. Continuing with the example of electronic activity 35152 illustrated above, in addition to extracting the timestamp feature, the electronic activity parser 210 can extract a first feature corresponding to a recipient of the electronic activity 35152, a second feature corresponding to a location of the appointment, and so on.

The method 3600 may include determining, for each electronic activity of the plurality of electronic activities, a relevancy score between the electronic activity and the scheduled event based on the extracted feature set (BLOCK 3608). In some embodiments, the electronic activity parser 210 can use a feature set, including one or more features extracted from each of the electronic activities, to estimate, assign, or otherwise determine a relevance score for each of the electronic activities. In some implementations, the relevancy score may be a numerical value. For example, the relevance score may range from a first numerical value (e.g., “0”) to a second numerical value (e.g., “100”), where a higher relevancy score indicates that the electronic activity is more relevant to the event electronic activity. In some implementations, the numerical values may be integer values. In some other implementations, the numerical values may be decimal values (e.g., decimal values between 0 and 1). In some implementations, the relevance score may be represented by a limited number of symbolic values (e.g., “LOW,” “MEDIUM,” “HIGH”), where LOW represents that the electronic activity is less relevant to the event electronic activity, MEDIUM represents that the electronic activity is relevant to the event electronic activity, and HIGH represents that the electronic activity is highly relevant to the event electronic activity.

The electronic activity parser 210 can determine the relevancy score for an electronic activity based on the feature(s) extracted from the electronic activity and/or a number of the extracted features. In some embodiments, the electronic activity parser 210 can determine the relevancy score for an electronic activity based on comparing the information parsed out from the event electronic activity (e.g., calendar-related, schedule-related, and/or meeting-related information) with the feature(s) extracted from the electronic activity. Based on the comparison, if there are a certain number of matches between the information of event electronic activity and the extracted feature(s) of the electronic activity (e.g., the number or degree of matching exceeds a threshold), the electronic activity parser 210 may assign a relatively high relevancy score to the electronic activity; and if there is no match between the information of event electronic activity and the extracted feature(s) of the electronic activity or the degree of matching does not meet or exceed a predetermined threshold, the electronic activity parser 210 may assign a relatively low relevancy score to the electronic activity.

With specific reference to the example of electronic activity 35152 above, the electronic activity parser 210 may assign a relatively high relevancy score (e.g., 90 out of 100) to the electronic activity 35152 based on the particular features extracted from the electronic activity 35152. For instance, in response to determining that the timestamp feature (e.g., 8 AM, Oct. 19, 2019, Pacific Time) is substantially close (e.g., within a predetermined time window threshold) to the time 3504 (9 AM Oct. 19, 2019, Pacific Time) of the event electronic activity 3502, the feature corresponding to the recipient of the electronic activity 35152 matches the organizer of the event electronic activity 3502, and the feature corresponding to a location extracted from the electronic activity 3502 matches or is near the location 3506 of the event electronic activity 3502, the electronic activity parser 210 may determine that the electronic activity 35152 is highly relevant to the event electronic activity 3502. Thus, the electronic activity parser 210 may assign a relatively high relevancy score to the electronic activity 35152. In another scenario, the electronic activity parser 210 may assign a relatively low relevancy score (e.g., 50 out of 100) to the electronic activity 35151 based on the features extracted from the electronic activity 35151. For instance, despite determining that a location feature (e.g., a destination referenced in the body of the email, such as “San Diego, California”) of the electronic activity 35151 matches or is near to the location 3506 (e.g., San Diego, California) of the event electronic activity 3502, the electronic activity parser 210 may still determine that the electronic activity 35151 is not particularly relevant to the event electronic activity 3502 based on one or more other features. For example, the electronic activity parser 210 can extract a timestamp feature from the electronic activity 35151 that corresponds to a timestamp at which a flight referenced in the electronic activity 35151 arrives in San Diego. The timestamp may be several days after the scheduled event (e.g., 11 AM, Oct. 21, 2019, Pacific Time). Accordingly, the electronic activity parser 210 can determine that the electronic activity 35151 is less related to the event electronic activity 3502, and thus may assign a relatively low relevancy score to the electronic activity 35151. In yet another scenario, the electronic activity parser 210 may assign a medium relevancy score (e.g., in the range of 40 to 70 out of 100) to the electronic activity 35151 based on the features extracted from the electronic activity 3515n. For instance, in response to determining that the recipient feature 3520n (e.g., a recipient of the electronic activity 3515n) matches a sender of the event electronic activity 3502 (e.g., an organizer of the scheduled event) and there is no other match, the electronic activity parser 210 may determine that the electronic activity 3515n is likely to be some somewhat relevant to the event electronic activity 3502, but may not conclusively determine a high degree of relevancy. Thus, the electronic activity parser 210 may assign a medium relevancy score to the electronic activity 3515n.

In some embodiments, the system 200, or a component thereof such as the electronic activity parser 210, can identify, for each of the plurality of electronic activities, a context of the electronic activity. For example, the system 200 can use a natural language processing (NLP) technique, a keyword search technique, or the like to extract one or more contexts for each electronic activity. Examples of contexts can include at least one of: a sender's or a recipient's willingness to attend a scheduled event, a sender's or a recipient's schedule at or near a time of the scheduled event, a sender's or a recipient's intended location at or near a time that is close to the scheduled event, etc. Upon identifying such context(s), the system 200 can determine a relevancy score between each of the electronic activities and the scheduled event using the identified context(s). For example, the system 200 can compare or determine whether any of the context(s) matches information of the scheduled event (e.g., the time 3504, the location 3505, etc.). If there are multiple matches, the system 200 can determine a relatively high relevancy score; if there is a single match, the system 200 can determine a medium relevancy score; and if there is no match, the system 200 can determine a relatively low relevancy score.

The method 3600 may include identifying a subset of electronic activities of the plurality of electronic activities that have respective relevancy scores satisfying a first threshold value (BLOCK 3610). In some embodiments, among the set of electronic activities corresponding to the electronic account of the first participant of the event electronic activity, the electronic activity parser 210 can select, filter, or otherwise identify a subset of the set of electronic activities based on the respective relevancy scores. As discussed above, the electronic activity parser 210 can assign a relevancy score to each of the set of electronic activities based on the respective feature set. The electronic activity parser 210 may compare the relevancy scores with a first threshold value. If the relevance score satisfies the first threshold value (e.g., greater than the first threshold value), the electronic activity parser 210 may select the corresponding electronic activity to be included in the subset; and if the relevance score does not satisfy the first threshold value (e.g., not greater than the first threshold value), the electronic activity parser 210 may not select the corresponding electronic activity to be included in the subset. In some embodiments, the system 200 may predefine the first threshold value as a static value.

Alternatively or additionally, the system 200 may dynamically adjust, tune, or otherwise update the first threshold value based on a number of the electronic activities identified for the participant of the event electronic activity, and/or a number of features extracted from each of the electronic activities.

Referring again to above examples where the electronic activity 35151 and electronic activity 35152 are assigned with the respective relevance scores, the electronic activity parser 210 can compare each of the relevance score with a threshold value, e.g., 60. In some embodiments, if the relevance score is greater than the threshold value, the electronic activity parser 210 may select the corresponding electronic activity as one of the subset of the electronic activities; and if the if the relevance score is equal to or less than the threshold value, the electronic activity parser 210 may not select the corresponding electronic activity as one of the subset of the electronic activities. For example, the electronic activity parser 210 may select the electronic activity 35152 to be included in the subset, responsive to determining that the relevance score corresponding to the electronic activity 35152 (90) is greater than 60. The electronic activity parser 210 may not select the electronic activity 35151 to be included in the subset, responsive to determining that the relevance score corresponding to the electronic activity 35151 (50) is less than 60. The electronic activity parser 210 may select the electronic activity 3515n to be included in the subset, responsive to determining that the relevance score corresponding to the electronic activity 3515n (70) is greater than 60.

In some embodiments, the electronic activity parser 210 can parse the subset of electronic activities to identify one of an electronic access values associated with the scheduled event (or the event electronic activity) or a physical address associated with the scheduled event (or the event electronic activity). Based on the identified electronic access value or the identified physical address associated with the scheduled event, the electronic activity parser 210 can determining an event type of the scheduled event. The electronic activity parser 210 can parse the event electronic activity 3502 to identify at least one of the following electronic access values associated with the event electronic activity 3502: dial-in information associated with the scheduled event, a uniform resource locator (URL) associated with the scheduled event (e.g., an address specifying the location of a networking resource for a virtual meeting room), a link or hyperlink to a virtual meeting room. Based on the identified electronic access value or the identified physical address, the electronic activity parser 210 can determine an event type of the scheduled event. For example, in response to identifying an electronic access value, the electronic activity parser 210 may determine that an event type of the scheduled event does not require the participants to physically attend the scheduled event. In another example, in response to identifying a physical address, the electronic activity parser 210 may determine that an event type of the scheduled event requires the participants to physically attend the scheduled event. In some instances, the electronic activity parser 210 may identify both an electronic access value associated with the scheduled event (or the event electronic activity) and a physical address associated with the scheduled event (or the event electronic activity). As such, the electronic activity parser 210 may determine that an event type of the scheduled event is a hybrid type, which requires part of the participants to physically attend the scheduled event and part of the participants to virtually (e.g., electronically) attend the scheduled event.

In some embodiments, the electronic activity parser 210 can determine that the scheduled event corresponds to a particular event type, responsive to determining that the event electronic activity satisfies an event type detection policy. The electronic activity parser 210 can periodically or continuously communicate or interface with an event type detection policy to determine an event type. The event type detection policy may include a plurality of previously identified or authorized URLs, links, or hyperlink, and a plurality of previously identified or authorized phone numbers (e.g., conference dial-in numbers). The electronic activity parser 210 can parse the event electronic activity 3502 to identify one or more electronic access values and compare the one or more electronic access values with the event type detection policy. In response to detecting or identifying a match, the electronic activity parser 210 can determine an event type for the event electronic activity 3502.

The method 3600 may include determining a probability score indicating a likelihood that the first participant attended the scheduled event based on the subset of electronic activities (BLOCK 3612). In response to identifying the subset of electronic activities with the respective relevance scores satisfying the first threshold value, the electronic activity parser 210 can use the subset of electronic activities and the corresponding feature sets to determine, estimate or otherwise assign a probability score. The probability score can indicate whether the first participant actually attended the scheduled event. The electronic activity parser 210 can determine the probability score based on a comparison between the event electronic activity and the subset of electronic activities. For example, the electronic activity parser 210 can compare the content(s) parsed out from the event electronic activity with the feature set(s) identified in the subset of the electronic activities. If there are multiple matches (e.g., exceeding a predefined number threshold) that tend to support a determination that the candidate participant attended the event, the electronic activity parser 210 can determine the probability score to be relatively high; if there are a limited number of matches (e.g., not exceeding the predefined number threshold) that tend to support a determination that the candidate participant attended the event or if there are one or more matches that support the determination that the candidate participant did not attend the event, the electronic activity parser 210 can determine the probability score to be relatively low. In instances in which it is unclear whether the candidate participant attended the scheduled event, the electronic activity parser can determine a medium probability score.

Continuing with the above example of FIG. 35 where the electronic activities 35152 and 3515n are selected in the subset of electronic activities, the electronic activity parser 210 can count the number of matches between the contents of the event electronic activity 3502 and the subset of electronic activities. For example, there are a total of 3 matches between the feature sets of the subset of the electronic activity and the contents (2804-2810) of the event electronic activity 3502 (e.g., the feature corresponding to the recipient of the electronic activity 35152 matches the organizer of the event electronic activity 3502, and the feature corresponding to the location of the electronic activity 35152 matches the location 3506 of the event electronic activity 3502, and the recipient feature of the electronic activity 3515n matches a sender of the event electronic activity 3502). Accordingly, the electronic activity parser 210 can determine that 3 is greater than a predefined number threshold, and can determine a probability score, indicating a likelihood that the first participant attended the scheduled event, to be relatively high.

In the case where the event electronic activity includes an electronic voice call, the system 200 can determine a probability score for the first participant based on a match between a phone number of the first participant and a phone number that participated in the electronic voice call. In some embodiments, the system 200 can receive, retrieve, or otherwise identify, from a data source provider corresponding to a host of the scheduled event, at least one phone number that participated in the electronic voice call. For example, the system 200 may integrate, interface, or otherwise communicate with a data source provider (e.g., a third-party company that provides video and/or audio conferencing services). The host of the scheduled event may schedule an electronic voice call using such a data source provider to provide a phone number for each of the participants to join (e.g., dial-in). Upon receiving the phone number, the system 200 can match the phone number with a number of the first participant to determine whether the first participant used his or her phone number to dial-in the phone number of the scheduled event. If the system 200 determines that there is a match, the system 200 can determine the probability score to be relatively high; and if the system 200 determines that there is no match, the system 200 can determine the probability score to be relatively low. Upon determining that there is a match, the system 200 can further determine, adjust, or otherwise update the probability score based on a time duration for which the first participant used his or her phone number to dial-in the phone number of the scheduled event.

In the case where the event electronic activity includes an electronic voice call, the system 200 can determine a probability score for the first participant based on a match between an acoustic fingerprint of an attendee of the electronic voice call (hereinafter “first acoustic fingerprint”) and an acoustic fingerprint of the first participant (hereinafter “second acoustic fingerprint”). In some embodiments, the system 200 can receive, retrieve, or otherwise access audio information corresponding to the electronic voice call. The system 200 can extract, select, or otherwise identify, from the audio information, the first acoustic fingerprint. The system 200 can identify, from the audio information, multiple acoustic fingerprints, and assign the acoustic fingerprints to respective attendees of the electronic voice call. Upon identifying the first acoustic fingerprint, the system 200 can match the first acoustic fingerprint with the second acoustic fingerprint to determine whether the first participant actually attended the scheduled event. If the system 200 determines that there is a match, the system 200 can determine the probability score to be relatively high; and if the system 200 determines that there is no match, the system 200 can determine the probability score to be relatively low.

In some embodiments, the system 200 can detect, identify, or otherwise determine a second subset of the plurality of electronic activities that were transmitted by the electronic account of the first participant during the scheduled event. The system 200 can generate a meeting engagement score based on a number of electronic activities included in the second subset. For example in FIG. 35, when the scheduled event in in progress, the system 200 can detect one or more electronic activities that participant A used the email address, A@ACME.COM, to communicate (e.g., one or more emails other than 35151 and 3515n). In an embodiment, upon detecting any of such a second subset of emails, the system 200 may initially assign an engagement score to participant A to be relatively low, and subsequently adjust (e.g., increase) the engagement score when determining that the second subset of emails were actually relevant to the scheduled event (or the event electronic activity 3502). For example, the system 200 can determine a percentage of the second subset of electronic activities that are relevant to the event. The system 200 can increase the engagement score in response to determining that the percentage is greater than or equal to a threshold; and the system 200 can decrease or maintain the engagement score in response to determining that the percentage is less than the threshold. For example, in some implementations, the system 200 can determine that an electronic activity is relevant to the event based on a determination that the electronic activity includes an attachment (e.g., a document or other file) related to the event. The system 200 can determine that the candidate participant had a high degree of engagement with the scheduled event if the candidate participant sent an electronic activity having a relevant document or file during the event. As a result, the system 200 may generate a relatively high engagement score. On the other hand, if the candidate participant sends electronic activities that are unrelated to the event during the event itself, the system 200 can determine that the candidate participant has a relatively low level of engagement with the event and generate a relatively low engagement score.

The method 3600 may include generating an attendance tag indicating that the first participant attended the scheduled event (BLOCK 3614). In some embodiments, upon determining the probability score, the electronic activity parser 210 may communicate or interface with the tagging engine 265 to determine whether the probability score satisfies a second threshold value. In response to determining that the probability score satisfies the second threshold value (e.g., greater than the second threshold value), the tagging engine 265 may assign an attendance tag to the event electronic activity indicating that the first participant actually attended the scheduled event. The tagging engine 265 can assign each of the participants of the event electronic activity based on a respective probability score for each of the participants of the event electronic activity. Continuing with the above example in FIG. 35, upon determining that the probability score, determined based on the subset of electronic activities 35152 and 3515n, satisfies the second threshold value, the tagging engine 265 can assign the attendance tag 3530 to the event electronic activity 3502 indicating that the first participant, A, actually attended the scheduled event.

In some embodiments, the tagging engine 265 can determine a confidence score for the attendance tag based on the probability score. The confidence score associated with the attendance tag can be indicative of a likelihood that the first participant actually attended the scheduled event. The tagging engine 265 can assign the confidence score to the attendance tag. In some embodiments, the tagging engine 265 can assign, estimate, or otherwise update the confidence score according to the probability score that is dynamically determined based on the above-discussed subset of electronic activities. For example, that tagging engine 265 may increase the confidence score in response to detecting an increase of the probability score. Similarly, that tagging engine 265 may decrease the confidence score in response to detecting a decrease of the probability score.

In some embodiments, the tagging engine 265 can update the confidence score using one or more electronic activities different from the event electronic activity. The system 200, or the electronic activity ingestor 205, can access a second, different electronic activity. The system 200, or the electronic activity parser 210, can extract, for the second, different electronic activity, a respective feature set. The feature set may include one or more features based on participants included in the second, different electronic activity or content included in a body of the second, different electronic activity. The system 200 can determine, based on the feature set of the second, different electronic activity, that a relevancy score between the second, different electronic activity and the scheduled event satisfies the first threshold value. Accordingly, the system 200 can update the confidence score of the attendance tag based on the second, different electronic activity.

In some embodiments, the tagging engine 265 can assign an occurrence tag to the event electronic activity based on the subset of electronic activities. The occurrence tag can indicate whether the scheduled event actually occurred. Based on the subset of electronic activities, the electronic activity parser 210 can determine a probability score indicating a likelihood that the scheduled event actually occurred. In response to determining that the probability score exceeds a predetermined event occurrence threshold, the tagging engine 265 can generate an occurrence tag indicating that the scheduled event occurred. Continuing with the above example in FIG. 35, upon determining that the probability score, exceeds the predetermined event occurrence threshold, the tagging engine 265 can assign the occurrence tag 3535 to the event electronic activity 3502 indicating that the scheduled event actually occurred. The tagging engine 265 can store, in a data structure, an association between the occurrence tag and the event electronic activity. In some embodiments, the tagging engine 265 can determine a confidence score for the occurrence tag based on the probability score. Upon determining the confidence score, the tagging engine 265 can assign the confidence score to the occurrence tag.

The system 200 can also use an occurrence tag for a scheduled event to update other information, such as one or more record objects. For example, an opportunity record object may correspond to a deal or other event whose outcome or status depends in part on the occurrence of the scheduled event. For example, if a scheduled event does not occur, it may indicate that a party to a deal (e.g., a buyer or a seller) is not engaged with the deal. Thus, if the occurrence tag does not exist for an event, or if the occurrence tag indicates that the event likely did not occur, the system 200 can adjust a status of the corresponding record object accordingly. On the other hand, if the occurrence tag indicates that the event likely did occur, the system 200 can adjust the corresponding record object to indicate that the deal has closed or that the stage or classification of the deal has advanced. Likewise, attendance of certain people may also influence the stage, classification, status, or completion of one or more record objects. Thus, in some implementations, the system 200 can adjust a record object (e.g., an opportunity record object) to indicate that that the deal has closed or that the stage or classification of the deal has advanced based on a determination that a particular candidate participant whose presence was important did attend the event, based on an attendance tag associated with a node profile representing the person.

In some embodiments, the electronic activity parser 210 can parse each of the subset of electronic activities to identify one or more of the candidate participants of the event electronic activity. Upon identifying the one or more of the candidate participants of the event electronic activity, the electronic activity parser 210 can determine whether the one or more of the candidate participants actually attended the scheduled event. In response to determining a number of the one or more of the candidate participants actually attending the scheduled event, the tagging engine 265 can update the confidence score of the occurrence tag. For example, the tagging engine 265 may determine that the number of candidate participants actually attending the scheduled event is less than a predefined threshold (e.g., 1). In response, the tagging engine 265 may determine that the scheduled event may never occur, and thus, may decrease the confidence score of the occurrence tag.

In some embodiments, the system 200 can further determine an attendance rate of the scheduled event based on the number of the candidate participants that actually attended the scheduled event. The system 200 can calculate the attendance rate based on a total number of the candidate participants associated with the event electronic activity and the number of the candidate participants that actually attended the scheduled event. For example, the attendance rate may be calculated as the number of the candidate participants that actually attended the scheduled event divided by the total number of the candidate participants associated with the event electronic activity. In response to determining the attendance rate, the system 200 can generate an attendance metric for the scheduled event. The system 200 can transmit data using the attendance metric to a computing device of an organizer of the scheduled event.

In some embodiments, the system 200 can access a performance profile of a node profile corresponding to the first participant. The system 200 can use an electronic account of the first participant to identify the node profile corresponding to the first participant from a node graph. As mentioned above, the node profile can include a plurality of node field-value pairs. Each node field-value pair can be associated with a value data structure corresponding to a value including entries identifying one or more data points. Each data point can be associated with a respective electronic activity or record object. In some embodiments, one or more of the plurality of node field-value pairs may constitute a performance profile. The performance profile may indicate one or more performance metrics of the first participant. Based on the attendance tag, the system 200 can update the performance profile by changing respective values of one or more of the node field-value pairs that constitute the performance profile. Continuing with the above example where the first participant is identified as being associated with the email address A@ACME.COM, the system 200 can identify a node profile corresponding to A@ACME.COM by identifying the value of an email field of the node profile matches A@ACME.COM. Upon identifying the node profile, the system 200 can update a performance profile of the node profile according to the attendance tag 3530. For example, in response to the tagging engine 265 generating the attendance tag 3530, the system 200 may increase one or more values of the node field-value pairs that constitute the performance profile. In some implementations, the performance profile can be adjusted based on part on a type of the scheduled event. For example, if the tagging engine 265 generates the attendance tag 3520 to indicate that the candidate participant attended an event that is relevant or important to the candidate participant's performance, such as a scheduled training session or an in-person meeting (e.g., a 1×1 meeting) with a superior, client, or other coworker, the system 200 can adjust the performance profile to indicate that the candidate participant has a higher level of performance, relative to a person who did not attend such an event. On the other hand, if the tagging engine 265 generates the attendance tag 3520 to indicate that the candidate participant attended an event that is less relevant or irrelevant the candidate participant's performance, such as a social event, a personal event, or another type of event that is not work-related, the system 200 may not make an adjustment to the performance profile.

In some embodiments, the system 200 can identify a first electronic activity of the subset of electronic activities of the first participant based on a communication string. Examples of the communication string may include one of: a dial-in phone number, a URL, a link, a hyperlink or the like. The system 200 can identify the first electronic activity, in response to determining that first electronic activity has a recipient identifier that matches a communication string included in the event electronic activity. The system 200 can parse the first electronic activity to identify the recipient identifier. In response to identifying the first electronic activity having the recipient identifier of the communication string, the tagging engine 265 can generate the attendance tag indicating that the first participant attended the scheduled event. In some embodiments, the first electronic activity may be an electronic voice call. In such cases, the system 200 can determine a duration of the electronic voice call. In response to determining that the duration of the call satisfies a duration threshold (e.g., greater than the duration threshold), the tagging engine 265 can generate the attendance tag indicating that the first participant attended the scheduled event. The duration threshold can be used to filter out calls that were made but not answered or calls that were answered by ended quickly (possibly because the recipient indicated they were busy at that time, or unavailable to talk).

In some embodiments, the system 200 can parse at least one of the subset of electronic activities sent or received within a predetermined time period of the scheduled event to determine a location associated with the at least one electronic activity. Upon determining the location, the system 200 can compare the location with a physical address associated with the event electronic activity (e.g., 3506). In some embodiments, the system 200 can determine the location by extracting, from the at least one electronic activity, an internet protocol (IP) address of a computing device of the first participant or a GPS location of the first participant within the predetermined time period. Based on the comparison, the tagging engine 265 can generate the attendance tag. Specifically, in response to determining, based on the comparison, that a proximity between the location and the physical address is within a predetermined proximity threshold, the tagging engine 265 can generate the attendance tag indicating that the first participant attended the scheduled event. For example in FIG. 35, the system may parse one of the electronic activities 35151-2815n (e.g., an email) sent to or received from a transportation networking company within a predetermined time period (e.g., 10 minutes) of a starting time the scheduled event, 9 AM, Oct. 19, 2019, Pacific Time (e.g., sometime between 8:50 AM and 9:10 AM, Oct. 19, 2019, Pacific Time). The email can be parsed to indicate that the participant A may be currently located at a location, e.g., Del Mar, California, that is substantially close to the location 3508 of the scheduled event, San Diego, California. As such, the tagging engine 265 can generate an attendance tag indicating that the participant actually attended the scheduled event.

In some embodiments, the system 200 can receive, retrieve, or otherwise identify travel information for the first participant. The travel information may correspond to travel within a predetermined time period of the scheduled event. The system 200 can interface, integrate, or communicate with a data source provider (e.g., a travel agency) to receive such travel information. The system 200 can extract a location from the travel information. Upon determining the location, the system 200 can compare the location with a physical address associated with the event electronic activity (e.g., 3506). Based on the comparison, the tagging engine 265 can generate the attendance tag. Specifically, in response to determining, based on the comparison, that a proximity between the location and the physical address is within a predetermined proximity threshold, the tagging engine 265 can generate the attendance tag indicating that the first participant attended the scheduled event. For example in FIG. 35, the system may communicate with a transportation networking company within a predetermined time period (e.g., 10 minutes) of a starting time the scheduled event, 9 AM, Oct. 19, 2019, Pacific Time (e.g., sometime between 8:50 AM and 9:10 AM, Oct. 19, 2019, Pacific Time) to determine that the participant A may be arriving at a location, e.g., Del Mar, California, that is substantially close to the location 3508 of the scheduled event, San Diego, California. As such, the tagging engine 265 can generate an attendance tag indicating that the participant actually attended the scheduled event.

The method 3600 may include storing an association between the attendance tag and the event electronic activity (BLOCK 3616). The system 200 can store the association in one or more data structures. In some embodiments, the system 200 can detect changes in the stored associations between the attendance tag and the event electronic activity. For example, once a new electronic activity is identified as being associated with the event electronic activity, the system 200 can automatically update the attendance tag.

In some embodiments, the system 200 can access, for a second participant of the candidate participants, a plurality of electronic activities corresponding to an electronic account of the second participant. The system 200 can extract, for each of the plurality of electronic activities, a feature set including one or more features based on participants included in the electronic activity or content included in a body of the electronic activity. The system 200 can determine, for each of the plurality of electronic activities, a relevancy score between the electronic activity and the scheduled event based on the extracted feature set. The system 200 can identify a subset of electronic activities of the plurality of electronic activities that have respective relevancy scores that satisfy a second threshold value. The system 200 can determine a probability score indicating a likelihood that the second participant attended the scheduled event based on the subset of electronic activities. The system 200 can generate, responsive to the probability score not satisfying the second threshold value, an attendance tag indicating that the second participant did not attend the scheduled event. The system 200 can store, in the one or more data structures, an association between the attendance tag and the event electronic activity.

### 24. Estimating Time to Perform Electronic Activities

The present disclosure relates to systems and methods for estimating a time to perform electronic activities. In a given day, a person may send a number of emails or other electronic activity as a part of their employment. The person may rush through generation of the electronic activity, may copy material from other electronic activities, or may generate a brief electronic activity. Such types of electronic activity may be appropriate in some instances. However, in other instances, such types of electronic activity may be perceived as having a low quality. The systems and methods described herein can be configured to parse electronic activities to objectively determine a Quality of Electronic activity (QoE) score. In some implementations, the systems and methods described herein may determine the QoE score based on a determined language complexity of the electronic activity, based on a word/character count for the electronic activity, etc. As one example, a sender may generate and send an email to a recipient. The email may generally have a quality based on various objective characteristics of the email, such as the language complexity, the character/word count, an amount of content which was copy/passed from previous emails, whether the email contains typographical errors, whether the email was generated via a template, and so forth. The system may parse the email to identify the objective characteristics of the email. The system may generate a QoE score for the email based on such objective characteristics.

According to the embodiments of the systems and methods described herein, the system can parse electronic activity to objectively determine a quality of the parsed electronic activity. The system can assign the QoE score to electronic activity. The system can leverage various information included in the electronic activity to generate the QoE score of the electronic activity. By objectively determining and assigning QoE scores to electronic activities, users within a company can be objectively evaluated based on their electronic activity. Further, the system can assign a tag or score to a node profile associated with users based on a set of QoE scores. The tag may indicate, e.g., a level of engagement, responsiveness, carefulness, etc. of the user. The company can evaluate the user based on such tags assigned to corresponding node profiles for the user. Various other benefits and advantages of the present technical solution are apparent based on the description that follows.

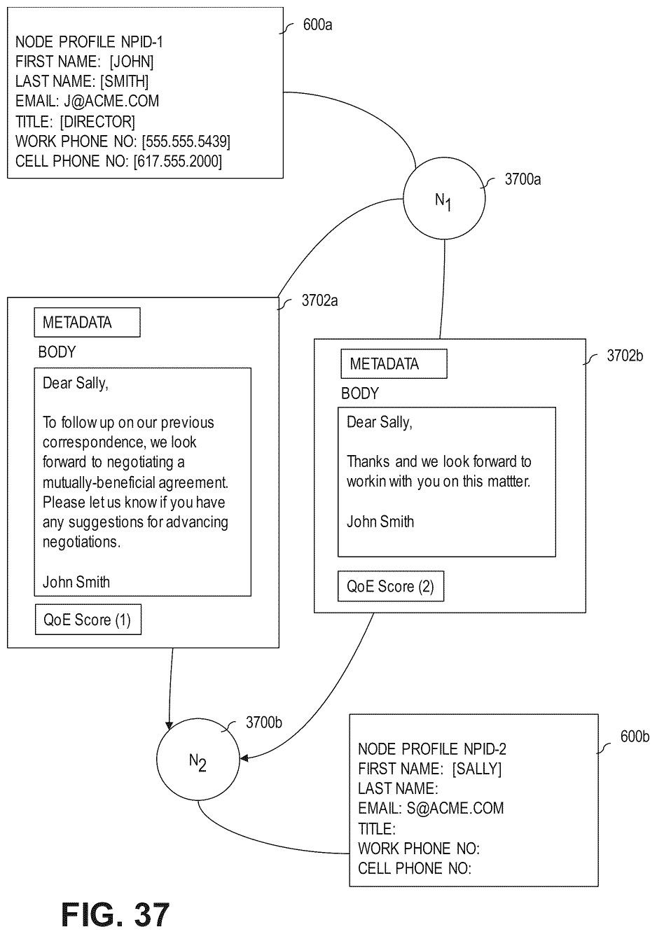

Referring now to FIG. 37, illustrated is a use case diagram of a plurality of electronic activities exchanged between two nodes 3700a, 3700b corresponding to respective users. Each user may include a corresponding node profile 600a, 600b. The node profiles 600 may correspond to a respective person or user. For instance, the node profile 600a may correspond to “John Smith”, node profile 600b may correspond to “John Doe”, and node profile 600b may correspond to “Sally”. As described in greater detail above, the system 200 may be configured to generate the node profiles 600a-600b based on electronic activity. In the use case diagram depicted in FIG. 37, a first user (e.g., “John Smith”) may generate and send one of a number of electronic activities to a second user (e.g., “Sally”). The use case diagram shows a first example electronic activity 3702a and a second electronic activity 3702b, though a given user may generate any number of alternative electronic activities. As described in greater detail below, the system 200 may be configured to parse the electronic activity 3702, and the system 200 may generate a QoE score for the electronic activity.

The system 200 may be configured to detect, receive, intercept, or otherwise identify electronic activities (such as electronic activity 3702) between two nodes 3700 corresponding to two persons. As described in greater detail above, the system 200 may include an electronic activity ingestor 205 which is configured to ingest electronic activities from the plurality of data source providers. The electronic activities may be received or ingested in real-time or asynchronously as electronic activities are generated, transmitted or stored by the one or more data source providers. The data source providers may be or include a server which hosts a domain corresponding one or more of the participants of the electronic activity (e.g., the sender or the recipient). The system 200 may identify electronic activities which identify a sender, one or more recipient(s), and a body including content. As described in greater detail below, the electronic activity parser 210 of the system 200 may be configured to parse the identified electronic activities. The electronic activity parser 210 may parse the electronic activities to identify objective characteristics corresponding to a quality of the electronic activity. The system 200 may be configured to generate a Quality of Electronic activity (QoE) score for each of the electronic activities transmitted by a given sender.

The system 200 may parse the electronic activity 3702 between the two nodes 3700. The electronic activity parser 210 may parse the header or metadata for the electronic activity 3702. As described in greater detail below, the electronic activity parser 210 may parse the header or metadata of the electronic activity 3702 to identify a sender and recipient(s) for the electronic activity 3702. Each electronic activity 3702 may include metadata or a header field which indicates or specifies an electronic account for a sender and an electronic account for each of the recipients. The electronic activity parser 210 may parse the electronic activity 3702 to identify the electronic accounts for the sender and recipient(s).

The node profile manager 220 may identify, from electronic activity, the values corresponding to a respective field for a node profile 600. As one example, where a node profile 600a corresponding to a node has not yet been generated and a person corresponding to the node generates and sends an email signed “John Smith”, the node profile manager 220 may first determine whether any node profiles 600 include field-value pairs including first name (John) and last name (Smith). When a matching node profile 600 is not identified, the node profile generator 220 may generate a node profile for “John Smith”. The node profile 600 may include field-value pairs for first name and last name based on the electronic activity. As another example, the node profile generator 220 may parse systems of record to identify matching node profiles.

By parsing the electronic activity, the electronic activity parser 210 may identify an electronic account associated with an electronic activity (e.g., the electronic activity 3702). The electronic activity parser 210 may identify the email address, phone number, etc. associated with a sender and recipient(s), for instance. The electronic activity parser 210 may identify the electronic account within a header field for the electronic activity. The electronic activity may specify a sender and recipient(s) in the header field. For instance, the electronic activity 3702 shown in FIG. 37 may include a header field specifying an electronic account corresponding to the recipient (e.g., “Sally”). The electronic activity parser 210 may identify the electronic account associated with the sender for purposes of assigning a tag to a corresponding node profile, as described in greater detail below. The node profile manager 220 may use the electronic account identified by or via the electronic activity parser 210 for identifying the node profile corresponding to the sender of the electronic activity. The node profile manager 220 may identify the node profile by performing a look-up function of the electronic account in several node profiles to locate a node profile having a matching electronic account.

In some embodiments, the electronic activity parser 210 may parse the metadata for the electronic activity 3702 to identify a timestamp for the electronic activity 3702. Each electronic activity 3702 may include metadata which includes a timestamp when the electronic activity 3702 is generated, sent, etc. The electronic activity parser 210 may parse the metadata to locate the field of the metadata corresponding to the timestamp. The electronic activity parser 210 may extract the timestamp corresponding to the electronic activity. In some implementations, the timestamp may be used for identifying, determining, or otherwise estimating the time taken to generate a corresponding electronic activity 3702, as described in greater detail below.

The electronic activity parser 210 may parse the body (e.g., content of the body) of the electronic activity 3702. The electronic activity parser 210 may parse the content of the electronic activity 3702 to identify various objective characteristics of the electronic activity 3702 indicative of a quality of the electronic activity 3702. Objective characteristics may include, for instance, a language complexity score for the electronic activity 3702, a word or character count for the electronic activity 3702, whether any typographical errors are present in the electronic activity 3702, how much of the electronic activity 3702 is reproduced from other electronic activities, whether the electronic activity 3702 is a blast electronic activity, and so forth.

The system 200 may be configured to generate a language complexity score indicating a level of language complexity of the electronic activity 3702. In some embodiments, the system 200 may include a language complexity determination engine. The language complexity determination engine may be any script, file, program, application, set of instructions, or computer-executable code that is configured to identify, determine, or otherwise generate a language complexity score. In some implementations, the language complexity determination engine may be embodied on the electronic activity parser 210. In some embodiments, the language complexity determination engine may parse the electronic activity 3702 to identify an average number of syllables per word, a number of words present in each sentence, etc. The language complexity determine engine may use the average number of syllables per word, word choices, grammar complexity, and/or number of words present in each sentence to generate the language complexity score. The language complexity score may be a score on any number of scales. For instance, the language complexity score may be on the Flesch Reading Ease scale, which calculates the readability of content on a scale ranging between 0-100 (with lower numbers indicating content is more difficult to comprehend). As another example, the language complexity score may be on the Flesch-Kincaid Grade scale, which indicates how many years of education needed to comprehend the content.

Specifically referring to the example of FIG. 37, the sender may generate either the first or second electronic activity 3700a, 3700b. The first electronic activity 3700a has longer sentences, words with a greater number of syllables, and has increased grammar complexity. The second electronic activity 3700b has short sentences constructed of words with fewer syllables and basic grammar. The language complexity score of the first electronic activity 3700a may therefore be greater than the language complexity score of the second electronic activity 3700b, as the first electronic activity 3700a is more difficult to read than the second electronic activity 3700b.

The system 200 may be configured to determine a character or word count of the electronic activity 3702. In some embodiments, the system 200 may include a word counting engine and/or character counting engine. The word counting engine may be any script, file, program, application, set of instructions, or computer-executable code that is configured to determine a word count for a body of an electronic activity 3702. Similarly, the character counting engine may be any script, file, program, application, set of instructions, or computer-executable code that is configured to determine a character count for a body of an electronic activity 3702. In some implementations, the word counting engine and/or character counting engine may be embodied on the electronic activity parser 210. The character counting engine may count each character in the body (e.g., letters, numbers, punctuation, spaces, etc.). The word counting engine may group character(s) together which are separated by a space or spaces, and count each group as one word.

Continuing the example in FIG. 37, the first electronic activity 3700a and second electronic activity 3700b include a different number of words and characters. The word (or character) counting engine may count the number of words in the first electronic activity 3700a and second electronic activity 3700b. The word (or character) counting engine may determine that the first electronic activity 3700a has a greater number of words and characters than the second electronic activity 3700b.

The system 200 may be configured to determine an estimated amount of time taken to generate the electronic activity 3702 using the language complexity score, the character count, and/or the word count. In some embodiments, the system 200 may include a time estimation engine. The time estimation engine may be any script, file, program, application, set of instructions, or computer-executable code that is configured to determine an estimated amount of time to generate the electronic activity. In some implementations, the time estimation engine may be embodied on the electronic activity parser 210. The time estimation engine may be configured to store, access, or otherwise use a formula for computing the estimated amount of time to generate the electronic activity. The time estimation engine may use the language complexity score and/or character/word count as inputs to the formula. The time estimation engine may retrieve the language complexity score from the language complexity determination engine and the word/character count from the word counting engine/character counting engine. The time estimation engine may apply the formula to the language complexity score and character/word count to compute the estimated time. In some embodiments, the time estimation engine may multiply the character/word count by a constant (e.g., a fixed number of seconds per word or character) and apply a multiplier based on the language complexity score. The multiplier may increase the value corresponding to the estimated time in proportion to the language complexity score. In this regard, the estimated time may increase as the language complexity score indicates that the readability decreases to reflect that more difficult content to read takes a longer time to generate.

In some embodiments, the system 200 may be configured to determine the estimated amount of time taken to generate the electronic activity 3702 using a timestamp for the electronic activity 3702. In some embodiments, the electronic activity 3702 may be a reply to a previous electronic activity. The electronic activity parser 210 may be configured to parse the electronic activity to determine that the electronic activity 3702 is a response. In some embodiments, the electronic activity parser 210 may determine the electronic activity 3702 is a response to a previous electronic activity based on the subject line of the electronic activity 3702 beginning with “RE:.” As another example, the electronic activity parser 210 may determine the electronic activity 3702 is a response based on the electronic activity 3702 including a line break (e.g., and content beneath the line break). As yet another example, the electronic activity parser 210 may be configured to determine a context of the electronic activity 3702 and a context of a previous electronic activity by parsing the electronic activities using natural language understanding. The electronic activity parser 210 may determine that the electronic activity 3702 is a response based on the electronic activity 3702 sharing a context with a previous electronic activity.

As described above, the electronic activity parser 210 may identify a timestamp for the electronic activity 3702 and the timestamp for the previous electronic activity. The time estimation engine may determine the estimated time to generate the electronic activity based on the respective timestamps. In some implementations, the time estimation engine may determine the estimated time based on the timestamp corresponding to the response (e.g., electronic activity 3702) with respect to the timestamp of the previous (e.g., most recent) electronic activity. In some embodiments, the time estimation engine may select the computed estimated time based on the language complexity score and word/character count when the computed estimated time based on the language complexity score and word/character count is less than the computed estimated time based on the timestamps. In some embodiments, the time estimation engine may select the computed estimated time based on the language complexity score and word/character count when the computed estimated time based on the timestamp is less than the computed estimated time based on the language complexity score and word/character count. In this regard, the time estimation engine may select the lowest computed estimated time.

The time estimation engine may be configured to estimate the amount of time for generating an electronic activity based on other information from the electronic activity. The time estimation engine may include a model which uses various inputs for computing the estimated time. For instance, the model may use time of day, device, language, context, etc. as inputs for computing the estimated time for generating the electronic activity. Various examples of such inputs are described in greater detail below.

The time estimation engine may use the time of day for computing the estimated time for generating the electronic activity. The time estimation engine may identify the time of day in which the electronic activity was generated based on the timestamp for the electronic activity. The time estimation engine may use the time of day as an input to the model. The model may correlate various times of day with corresponding weights for computing the estimated time to generate the electronic activity. For instance, the weights for later in the day may cause the computed estimated time to increase to reflect a user's efficiency decreases over the course of a day. The time estimation engine may use the time of day as an input for computing the estimated time in accordance with the model.

The time estimation engine may use the device upon which the electronic activity was generated for computing the estimated time for generating the electronic activity. The time estimation engine may identify the device upon which the electronic activity was generated based on the metadata for the electronic activity, a signature, etc. As one example, the electronic activity parser 210 may parse the metadata for the electronic activity to identify an Internet Protocol (IP) address for the electronic activity. The electronic activity parser 210 may identify the device upon which the electronic activity was generated based on the IP address for the device. As another example, the electronic activity parser 210 may parse the body of the electronic activity to identify the device upon which the electronic activity was generated. The body of the electronic activity may state, for instance, “SENT FROM MY MOBILE DEVICE,” “SENT FROM MY TABLET,” etc. The electronic activity parser 210 may parse the body of the electronic activity to identify phrases within the electronic activity indicative of the device upon which the electronic activity was generated. The time estimation engine may use a device type corresponding to the device as an input to the model. The model may correlate various types of devices with corresponding weights for computing the estimated time to generate the electronic activity. For instance, the weight corresponding to a mobile device may cause the computed estimated time to increase to reflect that electronic activities typically take longer to generate on a mobile device. As another example, the weight corresponding to a tablet may cause the computed estimated time to decrease with respect to similar electronic activities generated on a mobile device to reflect that electronic activities may take less time to generate on a tablet than on a mobile device. The time estimation engine may use the device type as an input for computing the estimated time in accordance with the model.

The time estimation engine may use the language of the electronic activity for computing the estimated time for generating the electronic activity. The electronic activity parser 210 may parse the electronic activity to identify a language of the electronic activity. The electronic activity parser 210 may identify the language based on scripts used in the text (e.g., Latin, Cryllic, Greek, Arabic, etc.).

The electronic activity parser 210 may identify the language based on the scripts and word choices/spellings, grammars, or other semantic information obtained from the content of the electronic activity. The node profile corresponding to the sender may include a field-value corresponding to native language. The time estimation engine may use the native language and identified language for the electronic activity as an input to the model for computing the estimated time. The model may apply a first weight to electronic activity generated in the sender's native language, and a second weight to electronic activity generated in in a language different from the sender's native language.

The second weight may cause the computed estimated time to increase to reflect that electronic activities typically take longer to generate in a different language. The time estimation engine may use the identified language of the electronic activity as an input for computing the estimated time in accordance with the model.

The time estimation engine may use the context of the electronic activity for computing the estimated time for generating the electronic activity. The electronic activity parser 210 may parse the electronic activity using natural language processing to determine a context of the electronic activity. The electronic activity parser 210 may determine whether the context is related to business activities or personal activities. The time estimation engine may use the context of the electronic activity as an input to the model. The model may correlate various types contexts with corresponding weights for computing the estimated time to generate the electronic activity. For instance, the weight corresponding to personal contexts (e.g., a meeting for after work drinks or dinner) may cause the computed estimated time to decrease to reflect that personal electronic activities typically take less time to generate. As another example, the weight corresponding to business contexts may cause the computed estimated time to increase to reflect that work-related electronic activities may take more time to generate. The time estimation engine may use the context (or tags corresponding to determined contexts) as an input for computing the estimated time in accordance with the model.

The system 200 may be configured to generate a Quality of Electronic activity (QoE) score corresponding to an estimated quality of the electronic activity 3702. The system 200 may include a QoE scoring engine. The QoE scoring engine may be any script, file, program, application, set of instructions, or computer-executable code that is configured to determine, calculate, compute, or otherwise generate a QoE score which corresponds to an estimated quality of the electronic activity.

The QoE scoring engine may be embodied on the electronic activity parser 210. The QoE scoring engine may be configured to generate the QoE score in accordance with a quality estimation model. The quality estimation model may use various inputs for generating a QoE score. The QoE scoring engine may be configured to store, access, or otherwise use quality estimation model for computing the QoE score. The quality estimation model may use various inputs corresponding to objective information extracted from the electronic activity 3702 for computing the QoE score. The QoE scoring engine may apply weights to such inputs to compute the QoE score. For instance, the QoE scoring engine may apply a weight to the language complexity score to increase the QoE score as the language complexity score indicates in increase in complexity (or decrease in readability). As another example, the QoE scoring engine may apply a weight to the word or character count to increase the QoE score as the word/character count increases. Various other examples of how the QoE score may change based on objective information identified in the electronic activity 3702 are described in greater detail below.

In some embodiments, the system 200 may be configured to identify a job title corresponding to the sender and/or a job title corresponding to recipient of the electronic activity 3702. The node profile manager 220 may use the electronic account identified by the electronic activity parser 210 for the sender/recipient(s) to identify a node profile for the sender/recipient(s). The node profile manager 220 may perform a look-up function to cross-reference the identified electronic account with values from a field corresponding to “Electronic Account.” As described above, each node profile 600 may include a plurality of field-value pairs which correspond to information obtained from electronic activities. One field-value pair may correspond to electronic account. The node profile manager 220 may identify the node profile 600 for the sender and recipient(s) based on their corresponding electronic accounts having a matching value in a field-value pair of a node profile 600.

The node profiles 600 may include a field-value pair corresponding to job titles. The node profile manager 220 may identify the job title value for the recipient of the electronic activity 3702. The QoE scoring engine may be configured to identify compute an importance score based on the job title value of the recipient. In some implementations and as described in greater detail above, the system 200 may maintain a hierarchy of a company including corresponding job titles. The QoE scoring engine may determine the importance score based on a position of the job title value of the recipient within the hierarchy. The importance score may be a weight or value which changes based on job title. For instance, as a job title indicates a higher-level position in a company or organization, the importance score may correspondingly increase. The QoE scoring engine may compute the QoE score based on the importance score. The quality estimation model may use the importance score as an input for generation of the QoE score. The QoE scoring engine may determine the QoE score using the quality estimation model, which factors the importance score into computation of the QoE score. As such, the QoE score may increase as the importance score increases to reflect a user likely taking more time to generate an electronic activity to higher level persons.

In some implementations, the node profile manager 220 may identify the job title value for the sender of the electronic activity 3702. In some implementations, the node profile manager 220 may identify the job title value of the sender of the electronic activity in relation to the recipient of the electronic activity. The node profile manager 220 may determine a distance between the sender and recipient of the electronic activity within the hierarchy. The node profile manager 220 may determine the distance based on the number of job titles between the job title of the sender and the job title of the recipient. Hence, the distance may be a relative job title or position. The QoE scoring engine may be configured to identify or compute an importance score based on the relative position of the sender and recipient(s). For instance, as a job title of the recipient indicates a higher-level position in a company and a job title of the sender indicates a lower-level position in the company, the importance score may correspondingly increase. The QoE scoring engine may compute the QoE score based on the importance score. The QoE scoring engine may determine the QoE score by using the importance score as an input to the quality estimation model for computing the QoE score. The QoE score may increase as the importance score increases to reflect a user having a lower job title likely taking more time to generate an electronic activity to higher level persons within the same company.

In some embodiments, the system 200 may be configured to determine whether the electronic activity 3702 includes any typographical errors. The system 200 may include a spell-checking engine. The spell-checking engine may be any script, file, program, application, set of instructions, or computer-executable code that is configured to perform a spell and grammar check function on the content of the electronic activity 3702. The spell-checking engine may include or access a dictionary which is used to check spelling of various words within the content of the electronic activity. Particularly of relevance to FIG. 37, the first electronic activity 3700a does not include any spelling errors. However, the second electronic activity 3700b includes two spelling errors (e.g., “WORKIN” and “MATTTER”). The spell-checking engine may be configured to determine whether any typographical errors are present in the content of the electronic activity 3702. The quality estimation model may use the presence (and/or number) of typographical errors as an input for generation of the QoE score. The quality estimation model may use to the presence (and/or number) of typographical errors for proportionally changing the QOE score of the electronic activity. The quality estimation model may apply a weight which negatively impacts the QoE score of an electronic activity as the number of typographical errors increase. In this regard, the QoE score decreases as the number of typographical errors increases to reflect a lower quality electronic activity based on the presence of typographical errors.

In some implementations, the system 200 may be configured to compare electronic activity 3702 with other electronic activity. The node profile manager 220 may be configured to store electronic activity associated with a particular node profile 600. The node profile manager 220 may store electronic activity which is sent or received by an electronic account of a corresponding node profile 600. The node profile manager 220 may store the electronic activity corresponding to the node profile 600 in a data structure, such as a ledger or database. The node profile manager 220 may store, in the data structure, each of the electronic activity which is sent from the electronic account and electronic activity which is received by the electronic account. The node profile manager 220 may identify the node profile 600 by cross-referencing the electronic account of the electronic activity 3702 with a value of a field-value pair corresponding to electronic accounts in the node profiles 600. The node profile manager 220 may be configured to identify the data structure corresponding to the node profile 600 associated with the electronic account of the sender. The electronic activity parser 210 may compare the content of the electronic activity (e.g., electronic activity 3702) with previous electronic activity from the data structure. As described in greater detail below, the QoE scoring engine may generate a QoE score based on how much of the electronic activity 3702 is duplicated from a previous electronic activity.

In some implementations, the QoE scoring engine may compute the QoE score based on whether the sender copied content from a previous electronic activity. The electronic activity parser 210 may determine whether content from the electronic activity 3702 is copied from a previous electronic activity (e.g., generated by or received by the sender via their electronic account). The electronic activity parser 210 may determine whether the content is copied from a previous electronic activity based on the comparison of the content to content of previous electronic activities in the ledger/database. The electronic activity parser 210 may compute the number of instances in which the content is the same. The electronic activity parser 210 may assign a copying score to the electronic activity which correlates the copying score to the number of instances in which the content is the same. As one example, an electronic activity which has a sentence that is the same as a previous electronic activity may have a first copying score, and an electronic activity which has a paragraph which is the same as a previous electronic activity may have a second copying score which is higher than the first. The copying score may increase as the number of instances increases. The Quality estimation model may use the copying score as an input for computing the QoE score. Hence, the QoE scoring engine may compute the QoE score based on the copying score for the electronic activity. The Quality estimation model may apply a weight to the copying score which cause the QoE score to decrease as the copying score increases. In other words, the QoE scoring engine may compute the QoE score based on the difference in content between the electronic activity 3702 and previous electronic activities.

In some instances, most of the content of the electronic activity 3702 may be the same as a previous electronic activity. For instance, the sender may generate the electronic activity using a template. The content may be the same as the content of the previous electronic activity except for a few terms (e.g., recipient(s) name, date, and/or other fillable form fields). The electronic activity parser 210 may determine that the electronic activity 3702 was generated via a template in a number of different ways. In some embodiments, the electronic activity 3702 may include metadata which indicates that the electronic activity 3702 was generated via a template. The electronic activity parser 210 may identify the metadata for the electronic activity 3702 to determine whether the metadata indicates the electronic activity 3702 was generated via the template. In some embodiments, the electronic activity parser 210 may determine that the electronic activity 3702 is generated based on the copying score. The electronic activity parser 210 may compare the copying score to a predetermined value corresponding to a template. The predetermined value may be a value which corresponds to a number of matching content that indicates the electronic activity was generated via a template. For instance, where two electronic activities are generated via a template, the content of such electronic activity may be the same in many portions. The copying score for such electronic activities may be greater than or equal to the predetermined value. As another example, the predetermined value may correspond to a number of differences between the electronic activities. The electronic activity parser 210 may determine that the electronic activity 3702 was generated via a template based on the number of differences between the electronic activities being less than the predetermined value. The electronic activity parser 210 may determine that the electronic activity 3702 was generated via a template based on the copying score in comparison to the predetermined value.

The quality estimation model may use, as an input, the determination that the electronic activity 3702 was generated via a template for computing the QoE score. The electronic activity 3702 being generated via a template may negatively impact the QoE score such that the QoE score of the electronic activity 3702 decreases where the electronic activity 3702 decreases. The QoE scoring engine may compute the QoE score based on whether the electronic activity 3702 was generated via a template.

In some embodiments, the QoE scoring engine may determine whether the electronic activity 3702 is one of a plurality of electronic activities sent as a part of a “blast” email. The electronic activity parser 210 may identify whether the sender sent a plurality of electronic activities (e.g., including the electronic activity 3702) within a predetermined time (e.g., a number of minutes, for instances). The electronic activity parser 210 may compare the content of the electronic activities to determine a difference in content for the electronic activities (e.g., in a manner similar to the comparison described above). Where the electronic activities are determined to have few differences (e.g., similar content), the electronic activity parser 210 may determine that the electronic activities are all related. The electronic activity parser 210 may determine that the electronic activities are a part of a “blast” email to a plurality of recipients. In some embodiments, the tagging engine 265 may be configured to apply a blast email tag to the electronic activity 3702 (e.g., as described above in section 1(g)). The QoE scoring engine may determine the QoE score based on the blast email tag applied to the electronic activity 3702. The quality estimation model may use tags (such as the blast email tag) assigned to the electronic activity 3702 as inputs for modifying/changing the QoE score. For instance, the blast email tag may negatively impact the QoE score. The QoE scoring engine may compute the QoE score based on the tag assigned to the electronic activity.

The system 200 may be configured to store the QoE score for the electronic activity 3702 in a data structure (such as the database or ledger) in association with the QoE score. Hence, each computed QoE score may be stored with the corresponding electronic activity. The QoE score may be used for generating reports, assigning tags or scores to corresponding node profiles 600, and so forth.

In some implementations, the tagging engine 265 may use the QoE scores for generating and assigning a tag to the node profile 600 associated with the sender. The tagging engine 265 may generate a tag which correlates the QoE scores for the electronic activities generated by the sender with a level of engagedness for the sender. The level of engagedness may be a value which indicates how engaged an employee is in their occupation. The level of engagedness may increase as the QoE scores increase, meaning that the sender is sending higher quality electronic activities and, as such, is more engaged in their occupation. The tagging engine 265 may compute a level of engagedness for the sender based on the QoE scores. The tagging engine 265 may compute the level of engagedness by averaging the QoE scores, applying a weight to more recent electronic activities, etc. The tagging engine may apply a time decay function to the QoE scores such that QoE scores for more recent electronic activity is favored in computing the level of engagedness. The tagging engine 265 may generate and apply a tag corresponding to the computed level of engagedness to the node profile 600 associated with the sender.

In some implementations, the system 200 may generate a report based on the QoE score for electronic activities. The system 200 may include a report generation engine. The report generation engine may be any script, file, program, application, set of instructions, or computer-executable code that is configured to generate and transmit a report corresponding to the sender. The report may correspond to a determined or predicted schedule for the sender. The report generation engine may identify a predetermined time window for generation of a report. The predetermined time window may be a day, a week, a month, etc. The report generation engine may generate a report for the predetermined window. The report generation engine may identify the electronic activity within the predetermined time window. The report generation engine may apply a time filter to the electronic activity stored in the data structure to remove those electronic activities outside the predetermined time window. The report generation engine may generate the report based on the QoE score for each of the electronic activities within the predetermined time window. The report generation engine may use the estimated time for generating the electronic activities for generation of the report. The report generation engine may use the estimated time for generating the electronic activity and the timestamp for the electronic activity for generating the report corresponding to the schedule of the sender. For instance, the report generation engine may use the timestamp of the electronic activity as the end of an entry on the report, and the estimated time for generating the report as the duration of the entry. Thus, the report generation engine may generate a report including entries which correspond to the estimated amount of time taken to generate various electronic activity.

In some implementations, the report generation engine may use other electronic activities (e.g., corresponding to calendar entries) for generating the report. The report generation engine may identify metadata, tags, etc. for other electronic activities (e.g., stored in the data structure or other locations accessible by the report generation engine) to identify those which correspond to a calendar or schedule for the user (such as meeting invitations, meeting notices, calendar entries, etc.). The report generation engine may incorporate such electronic activities into the report to accurately reflect the sender's schedule for the predetermined time window.

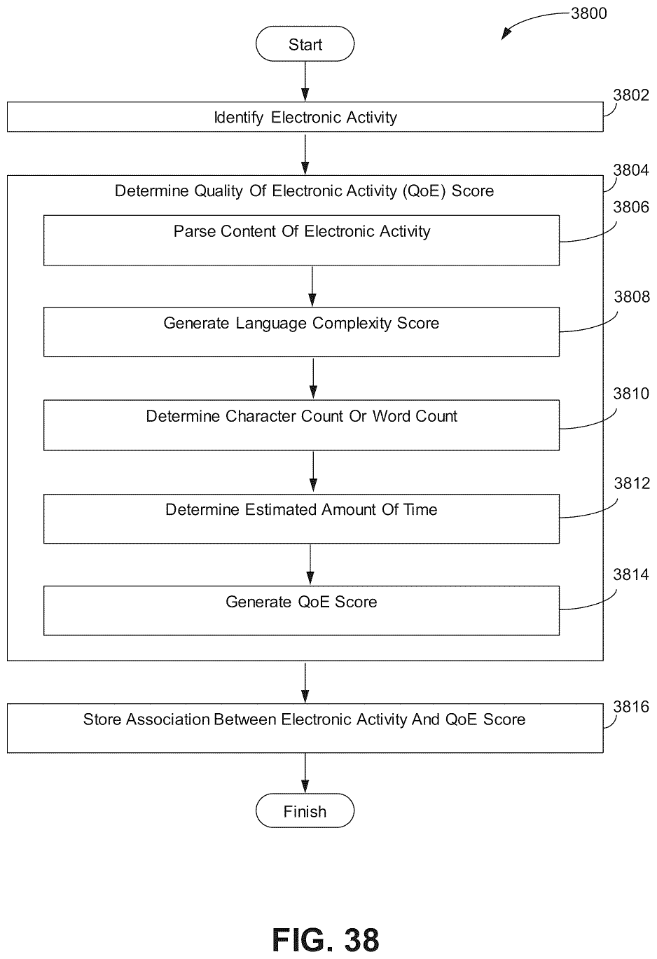

Referring now to FIG. 38, illustrated is a flow diagram of a method 3800 of estimating time to perform electronic activities. The method 3800 can be implemented or performed using any of the components described above in conjunction with FIGS. 1-28 (e.g., the node graph generation system 200) or the server system 4800 detailed below in conjunction with FIG. 48. The method 3800 may include identifying an electronic activity (3802). The method 3800 may include determining a Quality of Electronic activity (QoE) score (3804). Determining the QoE score may include parsing the content of the electronic activity (3806). Determining the QoE score may include generating a language complexity score (3808). Determining the QoE score may include determining a character count or word count (3810). Determining the QoE score may include determining an estimated amount of time (3812). Determining the QoE score may include generating the QoE score (3814). The method 3800 may include storing an association between the electronic activity and the QoE score (3816).

The method 3800 may include identifying an electronic activity (3802). In some embodiments, the system 200 may identify an electronic activity which identifies a sender and one or more recipient. The electronic activity may include a body having content. In some implementations, the electronic activity may be an email generated and sent by the sender to the recipient. Different electronic activity may have different quality. For instance, some electronic activity may be brief, contain typographical errors, be copied from other materials or sources, and so forth. Such electronic activity may have objectively lower quality than electronic activity which is original, developed, free of typographical errors, and so forth. The system 200 may ingest electronic activity from various sources, such as servers which host a domain associated with an electronic account for the sender and/or recipient(s). The system 200 may identify the electronic activity ingested from such sources that identifies a sender and recipient(s)

The method 3800 may include determining a Quality of Electronic activity (QoE) score (3804). In some embodiments, the system 200 may determine the QoE score using a quality estimation model. The QoE score may correspond to an estimated quality of the electronic activity. The quality estimation model may use various inputs for identifying the estimated quality of the electronic activity. For instance, the quality estimation model may use, as inputs, the length of the electronic activity (e.g., based on word or character count), a language complexity score, the presence of typographical errors, how much of the content is duplicated or copied from other electronic activities, whether the electronic activity is generated via a template, and so forth.

The method 3800 may include determining the QoE score by parsing the content of the electronic activity (3806). The electronic activity parser 210 may parse the content of the electronic activity. The electronic activity parser 210 may parse the body and/or metadata for the electronic activity. The electronic activity parser 210 may parse the electronic activity to identify various objective characteristics of the electronic activity. The system 200 may use the objective characteristics as inputs for the quality estimation model for computing the QoE score, as described in greater detail below.

The method 3800 may include determining the QoE score by generating a language complexity score (3808). The system 200 may use, maintain, or otherwise access a language complexity determination engine. The language complexity determination engine may compute the language complexity score for the electronic activity. The language complexity score may indicate a level of language complexity. The language complexity determination engine may parse the electronic activity to identify an average number of syllables per word, a number of words present in each sentence, etc. The language complexity determine engine may include a policy which uses the average number of syllables per word, word choices, grammar complexity, and/or number of words present in each sentence to generate the language complexity score. The language complexity score may be a score on any number of scales. For instance, the language complexity score may be on the Flesch Reading Ease scale, which calculates the readability of content on a scale ranging between 0-100 (with lower numbers indicating content is more difficult to comprehend). As another example, the language complexity score may be on the Flesch-Kincaid Grade scale, which indicates how many years of education needed to comprehend the content. The system 200 may use the language complexity score as an input for the quality estimation model in computing the QoE score.

The method 3800 may include determining the QoE score by determining a character count or word count (3810). The system 200 may include a word or character counting engine which count each character in the body (e.g., letters, numbers, punctuation, spaces, etc.). The word counting engine may group character(s) together which are separated by a space or spaces, and count each group as one word. The system 200 may count the words/characters within the electronic activity. The system 200 may use the word/character count as an input for the quality estimation model in computing the QoE score.

The method 3800 may include determining the QoE score by determining an estimated amount of time (3812). The system 200 may determine the estimated amount of time using the language complexity score and the character/word count. In some embodiments, the time estimation engine may compute the estimated amount of time using a formula which uses the character/word count and the language complexity score for computing the estimated amount of time. The time estimation engine may retrieve the language complexity score from the language complexity determination engine and the word/character count from the word counting engine/character counting engine. The time estimation engine may apply the formula to the language complexity score and character/word count to compute the estimated time. In some embodiments, the time estimation engine may multiply the character/word count by a constant (e.g., a fixed number of seconds per word or character) and apply a multiplier based on the language complexity score. The multiplier may increase the value corresponding to the estimated time in proportion to the language complexity score.

In some embodiments, the system 200 may determine the estimated amount of time based on timestamps for the electronic activity. For instance, the electronic activity may be a response to a first electronic activity. The time estimation engine may determine a timestamp for each of the first electronic activity and the response (e.g., second electronic activity). The time estimation engine may calculate a difference between the timestamp for the second electronic activity and the timestamp for the first electronic activity. The time estimation engine may determine the estimated amount of time by computing the difference between the first and second timestamp associated with the first electronic activity and the response.

The time estimation engine may be configured to estimate the amount of time for generating an electronic activity based on other information from the electronic activity. The time estimation engine may include a model which uses various inputs for computing the estimated time. For instance, the model may use time of day, device, language, context, etc. as inputs for computing the estimated time for generating the electronic activity. Various examples of such inputs are described in greater detail below.

The time estimation engine may use the time of day for computing the estimated time for generating the electronic activity. The time estimation engine may identify the time of day in which the electronic activity was generated based on the timestamp for the electronic activity. The time estimation engine may use the time of day as an input to the model. The model may correlate various times of day with corresponding weights for computing the estimated time to generate the electronic activity. For instance, the weights for later in the day may cause the computed estimated time to increase to reflect a user's efficiency decreases over the course of a day. The time estimation engine may use the time of day as an input for computing the estimated time in accordance with the model.

The time estimation engine may use the device upon which the electronic activity was generated for computing the estimated time for generating the electronic activity. The time estimation engine may identify the device upon which the electronic activity was generated based on the metadata for the electronic activity, a signature, etc. As one example, the electronic activity parser 210 may parse the metadata for the electronic activity to identify an Internet Protocol (IP) address for the electronic activity. The electronic activity parser 210 may identify the device upon which hthe electronic activity was generated based on the IP address for the device. As another example, the electronic activity parser 210 may parse the body of the electronic activity to identify the device upon which the electronic activity was generated. The body of the electronic activity may state, for instance, “SENT FROM MY MOBILE DEVICE,” “SENT FROM MY TABLET,” etc. The electronic activity parser 210 may parse the body of the electronic activity to identify phrases within the electronic activity indicative of the device upon which the electronic activity was generated. The time estimation engine may use a device type corresponding to the device as an input to the model. The model may correlate various types of devices with corresponding weights for computing the estimated time to generate the electronic activity. For instance, the weight corresponding to a mobile device may cause the computed estimated time to increase to reflect that electronic activities typically take longer to generate on a mobile device. As another example, the weight corresponding to a tablet may cause the computed estimated time to decrease with respect to similar electronic activities generated on a mobile device to reflect that electronic activities may take less time to generate on a tablet than on a mobile device. The time estimation engine may use the device type as an input for computing the estimated time in accordance with the model.

The time estimation engine may use the language of the electronic activity for computing the estimated time for generating the electronic activity. The electronic activity parser 210 may parse the electronic activity to identify a language of the electronic activity. The electronic activity parser 210 may identify the language based on scripts used in the text (e.g., Latin, Cryllic, Greek, Arabic, etc.).

The electronic activity parser 210 may identify the language based on the scripts and word choices/spellings, grammars, or other semantic information obtained from the content of the electronic activity. The node profile corresponding to the sender may include a field-value corresponding to native language. The time estimation engine may use the native language and identified language for the electronic activity as an input to the model for computing the estimated time. The model may apply a first weight to electronic activity generated in the sender's native language, and a second weight to electronic activity generated in in a language different from the sender's native language.

The second weight may cause the computed estimated time to increase to reflect that electronic activities typically take longer to generate in a different language. The time estimation engine may use the identified language of the electronic activity as an input for computing the estimated time in accordance with the model.

The time estimation engine may use the context of the electronic activity for computing the estimated time for generating the electronic activity. The electronic activity parser 210 may parse the electronic activity using natural language processing to determine a context of the electronic activity. The electronic activity parser 210 may determine whether the context is related to business activities or personal activities. The time estimation engine may use the context of the electronic activity as an input to the model. The model may correlate various types contexts with corresponding weights for computing the estimated time to generate the electronic activity. For instance, the weight corresponding to personal contexts (e.g., a meeting for after work drinks or dinner) may cause the computed estimated time to decrease to reflect that personal electronic activities typically take less time to generate. As another example, the weight corresponding to business contexts may cause the computed estimated time to increase to reflect that work-related electronic activities may take more time to generate. The time estimation engine may use the context (or tags corresponding to determined contexts) as an input for computing the estimated time in accordance with the model.

The method 3800 may include generating the QoE score (3814). The system 200 may generate the QoE score corresponding to the estimated quality based on the estimated amount of time. The system 200 may use the estimated amount of time as an input to the quality estimation model for computing the QoE score. The QoE score may reflect an objective quality of the electronic activity. The QoE score may increase as the objective characteristics of the electronic activity indicate that the electronic activity has a greater quality. Where the language complexity score and word/character count is increased, the objective quality of the electronic activity correspondingly increases. Other inputs to the quality estimation model may be used for computing the QoE score of the electronic activity.

In some embodiments, the quality estimation model may use an importance score for the recipient for generation of the QoE score. The system 200 may determine a node profile corresponding to the recipient. The system 200 may determine the node profile for the recipient by performing a look-up function on node profiles using the electronic account for the recipient specified in the electronic activity. The system 200 may identify a job title value associated with a field-value pair for the node profile for the recipient. The system 200 may generate an importance score based on the job title of the recipient. The system 200 may maintain a hierarchy for the company or enterprise in which the recipient is employed. The system 200 may include or use a policy which correlates various job title values within the hierarchy with corresponding importance scores. The system 200 may use the policy for identifying the importance score for the job title value of the recipient. The quality estimation model may use the importance score as an input for computing the QoE score.

In some embodiments, the quality estimation model may use the importance score for the recipient and an importance score for the sender for generating the QoE score. Similar to generation of the importance score for the recipient described above, the system 200 may determine a node profile for the sender by performing a look-up function on node profiles using the electronic account for the sender specified in the electronic activity. The system 200 may identify a job title value associated with a field-value pair for the node profile for the sender. The system 200 may generate an importance score based on the job title for the sender by identifying the importance score correlated with the job title value within the hierarchy for the sender's company or enterprise. The system 200 may compare the importance score for the sender and the importance score of the recipient. The relative importance scores may be inputs to the quality estimation model for generation of the QoE score. For instance, where the sender has a higher importance score than the recipient, the relative importance score may negatively impact the QoE score, as the sender likely took less time generating the electronic activity. As another example, where the sender has a lower importance score than the recipient, the relative importance score may positively impact the QoE score, as the sender was likely more careful in generating the electronic activity.

In some embodiments, the quality estimation model may use a presence of typographical errors in the content of the electronic activity as an input for generating the QoE score. The system 200 may include or maintain a spell-checking engine. The spell-checking engine may include or access a dictionary which is used to check spelling of various words within the content of the electronic activity. The spell-checking engine may determine whether any typographical errors are present in the content of the electronic activity. The quality estimation model may use the presence (and/or number) of typographical errors as an input for generation of the QoE score. The quality estimation model may use to the presence (and/or number) of typographical errors for proportionally changing the QoE score of the electronic activity. The Quality estimation model may apply a weight which negatively impacts the QoE score of an electronic activity as the number of typographical errors increase. In this regard, the QoE score decreases as the number of typographical errors increases to reflect a lower quality electronic activity based on the presence of typographical errors.

In some embodiments, the quality estimation model may use an amount or size of content of the electronic activity which is was previously transmitted or received by the electronic account of the sender of the electronic activity for computing the QoE score. As described in greater detail below, the system 200 may maintain a data structure including electronic activity (or activities) associated with the sender. The system 200 may determine that a portion of the content included in the electronic activity is included in a plurality of electronic activities previously transmitted or received by an electronic account of the sender of the electronic activity. The system 200 may compare the content of the electronic activity with the content from previous electronic activities. The system 200 may determine the size of the portion of the electronic activity which is included in the previous electronic activity. The quality estimation model may use the size of the portion as an input for generating the QoE score. For instance, where the size of the portion of the electronic activity that is included in a previous electronic activity increases, the QoE score may correspondingly decrease to reflect that the sender copied a larger portion of the content from a previous electronic activity.

In some embodiments, the quality estimation model may use the difference in content between the electronic activity and at least one other electronic activity for computing the QoE score. The system 200 may compare the electronic activity with at least one other electronic activity transmitted or received by the electronic account of the sender of the electronic activity to determine a difference in the content between the electronic activity and the at least one other electronic activity. The system 200 may determine a copying score based on the difference in content. The copying score may increase as the difference in content decreases (indicating few differences). The quality estimation model may use the copying score as an input for computing the QoE score. As such, the QoE score may be based on the difference in the content between the electronic activity and at least one other electronic activity.

In some embodiments, the quality estimation model may use a tag assigned to the electronic activity as an input for computing the QoE score. The system 200 may identify a plurality of other electronic activities transmitted by the sender within a predetermined time window of the electronic activity. The predetermined time window may be a number of minutes, hours, etc., which corresponds to sending a “blast” email. The system 200 may compare the electronic activity with the other electronic activities to determine a difference between the respective electronic activities. The system 200 may determine that the difference between the electronic activities satisfies a predetermined threshold indicating that the electronic activities are very similar. The system 200 may assign a tag to each of the electronic activities indicating that the electronic activities are blast emails. The system 200 may assign the tag responsive to determining that the difference in content satisfies the predetermined threshold. The quality estimation model may use the presence of the tag for generation of the QoE score such that, where the tag is present, the QoE score decreases.

The method 3800 may include storing an association between the electronic activity and the QoE score (3816). In some embodiments, the system 200 may store the association between the electronic activity and the QoE score in a data structure, such as a database, a ledger, and so forth. The system 200 may store the association between the electronic activity and QoE score for a predetermined duration (e.g., a month, a year, a number of years, indefinitely, etc.). The system 200 may use the QoE score and electronic activity for performing various functions using the QoE scores.

In some embodiments, the system 200 may identify a plurality of QoE scores corresponding to the sender of the electronic activity. The data structure may include a correlation with the node profile for the sender. The system 200 may identify the QoE scores for the sender by cross-referencing the electronic account for the sender with the data structure to identify the QoE scores for the sender. The system 200 may generate and assign a tag to the node profile for the sender based on the QoE scores.

The system 200 may generate the tag by computing an average of the QoE scores. The system 200 may apply a time decay function which decreases the QoE scores over time to favor the QoE scores for more recent electronic activities. The system 200 may assign the tag to the node profile associated with the sender based on the plurality of QoE scores.

In some embodiments, the system 200 may identify a plurality of electronic activities transmitted by the sender within a predetermined time window (e.g., within the same day, within the same week, within the same month, etc.). The system 200 may generate a QoE score for each of the plurality of electronic activities within the predetermined time window. The system 200 may generate a report corresponding to a schedule of the sender based on the QoE score of each of the electronic activities within the predetermined time window. The system 200 may use the QoE scores for identifying entries in the schedule of the sender. For instance, the system 200 may identify the timestamp for the corresponding electronic activities, and the estimated amount of time for generating an entry. The system 200 may compile the report using the timestamp and estimated amount of time. In some embodiments, the system 200 may use other electronic activities for incorporating further entries into the report. The system 200 may identify electronic activities corresponding to calendar entries (e.g., meeting notices, meeting invitations, etc.) within the predetermined time window. The system 200 may generate the report corresponding to the schedule of the sender based on the other electronic activities corresponding to calendar entries, the QoE score of the electronic activity and the QoE scores of electronic activities within the predetermined time window.

### 25. Updating Email Addresses Based on Email Generation Patterns

The present disclosure relates to systems and methods for updating email addresses based on email generation patterns. As described in the systems and methods herein, electronic activities can be matched to node profiles to improve the accuracy of activity field-value pairs of node profiles. In some instances, as new electronic activities are processed, it is possible that the system may identify values from some of the electronic activities that do not provide electronic account identifiers to be matched to any of the existing and new node profiles of the system. Such “missing” electronic account identifiers in the node profiles may cause a data source provider that utilizes the system to manage the electronic activities to miss a deal, a deadline, up-to-date information, among others. Accordingly, the disclosed system provides a technical solution to automatically determine, generate, or otherwise update one or more electronic account identifier patterns. The system can determine an electronic account identifier pattern by identifying, for each member node profile of a group entity (e.g., a company, or a data source provider), a correlation between the respective electronic account identifier, first name, and last name. The system can automatically update a member node profile, that is determined to miss an activity field-value pair corresponding to the electronic account identifier, to include one generated based on the electronic account identifier pattern. Further, the system can detect an operating or ownership change to the group entity (e.g., mergers and acquisitions), and based on the detected change, update a respective activity field-value pair corresponding to the electronic account identifier of each of the member node profiles. Thus, the disclosed system can dynamically and accurately update each of the member node profiles with the most up-to-date activity field-value pair corresponding to the electronic account identifier.

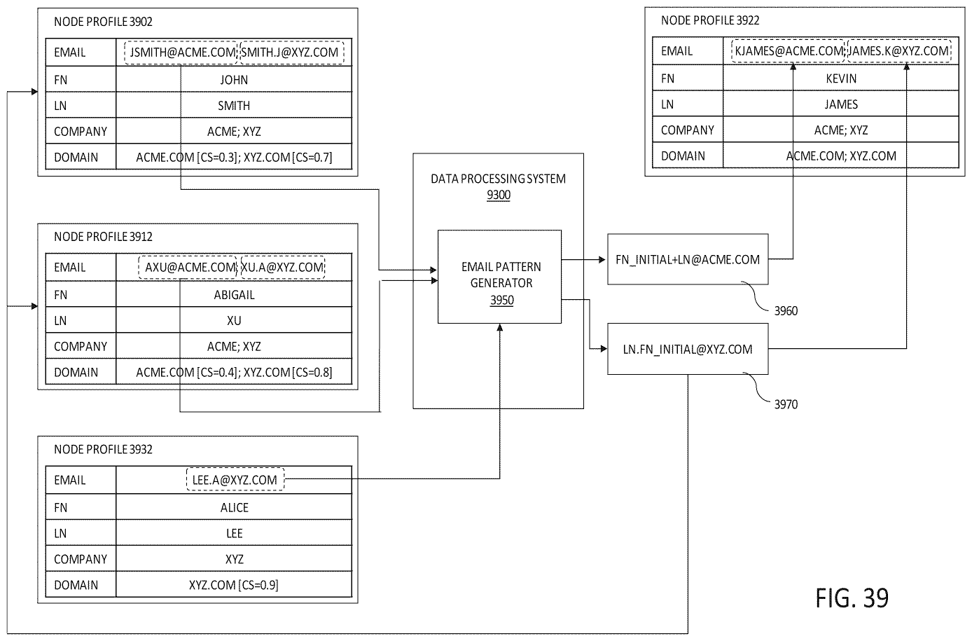

Referring now to FIG. 39, FIG. 39 illustrates a block diagram of an example process flow for updating electronic account identifiers (e.g., email addresses) based on email generation patterns. For the purposes of describing systems and methods for updating email addresses based on email generation patterns, the present disclosure relies in part on FIGS. 3-9 and 25-26, as described above. For example, the data processing system 9300 can be configured to identify respective activity field-value pairs corresponding to the email address from a plurality of member node profiles of a group entity. Based on the identified values of the email address, the data processing system 9300 can determine an email generation pattern by correlating the email address to the one or more other activity field-value pairs corresponding to respective first name and last name. Using the email generation pattern, the data processing system 9300 can update, fill up, or otherwise generate the value for an email address field of any of the member node profile of the group entity. Thus, the block diagram of FIG. 39 shall be described in conjunction with FIGS. 3-9 and 25-26.

In FIG. 39, two node profiles 3902 and 3912 are shown. Each of the node profiles 3902 and 3912 may correspond to or represent a person of a group entity. Accordingly, the node profiles 3902 and 3912 can each be a member node profile linked or associated to a group node profile of the group entity. Each of the node profiles 3902 and 3912 can include a plurality of field-value pairs. Each field-value pair can be associated with a corresponding field and one or more node field values. For example, the node profile 3902 includes an email field with a corresponding value, “JSMITH@ACME.COM (at a first timestamp);” a first name (FN) field with a corresponding value, “JOHN;” a last name (LN) field with a corresponding value, “SMITH;” a company name field with a corresponding value, “ACME (at the first timestamp);” and a domain name field with a corresponding value, “ACME.COM (at the first timestamp).” Similarly, the node profile 3912 includes an email field with a corresponding value, “AXU@ACME.COM (at the first timestamp);” a first name (FN) field with a corresponding value, “ABIGAIL;” a last name (LN) field with a corresponding value, “XU;” a company name field with a corresponding value, “ACME (at the first timestamp);” and a domain name field with a corresponding value, “ACME.COM (at the first timestamp).”

In some embodiments, the data processing system 9300 can include a pattern generator 3950 configured to determine one or more email address generation patterns based on the respective values of the email field, FN field, and LN field of each of the member node profiles. The data processing system 9300, or the pattern generator 3950, can identify the respective values of the email field, FN field, and LN field of each of the member node profiles, and determine a correlation between the values to determine an email address generation pattern. For example, the pattern generator 3950 can identify, for the member node profile 3902, the value of the email field JSMITH@ACME.COM is composed of a character string preceding a predetermined character (e.g., “@”) and a domain name (ACME.COM) following the predetermined character. For the member node profile 3902, the pattern generator 3950 can further identify that the character string is composed of an initial of the value of the FN filed (JOHN) followed by the value of the LN field (SMITH). Similarly, the pattern generator 3950 can identify, for the member node profile 3912, the value of the email field AXU@ACME.COM is composed of a character string preceding the character, @, and the identical domain name following the character, and further identify that the character string is composed of an initial of the value of the FN filed (ABIGAIL) followed by the value of the LN field (XU). As such, the pattern generator 3950 can determine, for the group entity (e.g., the company “ACME”), an email generation pattern 3960 to be “FN_INITIAL (an initial of the value of the FN field)” followed by “LN (the value of the LN field)” followed by “@” and followed by “ACME.COM (the value of the domain name field).”

In some instances, a newly generated member node profile may only include limited information about the node such as, for example, a first name, a last name, a company name, etc. Using the email generation pattern 3960, the pattern generator 3950 can generate, fill up, or otherwise provide the value of an email field-value pair of the new member node profile. For example, when the data processing system 9300 generated node profile 3922, the node profile 3922 may only have the values of the FN field, LN field, company name filed as “KEVIN,” “JAMES,” and “ACME,” respectively, while lacking the values of the email filed and domain name field. In response, the data processing system 9300 can use the pattern generator 3950 to generate a value for the email field of the node profile 3922. For example, the pattern generator 3950 can apply the email generation pattern 3960 to the values of FN filed and LN field to generate the value of the email field to be “KJAMES@ACME.COM”. Accordingly, the data processing system 9300, or the pattern generator 3950, can use the generated email to generate the value of the domain name field to be “ACME.COM.”

Overtime, the data processing system 9300 can identify another member node profile 3932 having a different domain name that belongs to another group entity but actually has a relationship to the group entity to which the node profiles 3902, 3912, and 3922 belong. In some embodiments, the data processing system 9300 can infer, update, or otherwise determine such a relationship by monitoring respective confidence scores (CSs) of the domain names of the two group entities. With specific reference to FIG. 39, at a second timestamp (later than the first timestamp), the data processing system 9300 can identify the node profile 3932 that includes values of the email field, FN field, LN field, company name field, and domain name field as “LEE.A@XYZ.COM,” “ALICE,” “LEE,” “XYZ,” and “XYZ.COM,” respectively. Based on electronic activities associated with the node profile 3932, the data processing system 9300 can determine a confidence score for the value of the domain name field to be a relatively high value, e.g., 0.9. Concurrently, the data processing system 9300 may identify that the confidence scores of the domain name field of the node profile 3902 and the domain name field of the node profile 3912 has decreased from higher values to relatively low values 0.3 and 0.4, respectively. As such, the data processing system 9300 can determine that the companies XYZ and ACME may have a relationship. For instance, the company ACME may be acquired, merged, or owned by the company XYZ at a certain timestamp between the first and second timestamps. In response to determining such a relationship, the data processing system 9300 can again use the pattern generator 3950 to generate another email generation pattern (e.g., 3970) to update the email field-value pairs of the node profiles 3902 and 3912 and/or generate an email field-value pair for the new node profile 3922. For example, based on the value of the email field of the member node profile 3932, “LEE.A@XYZ.COM,” the pattern generator 3950 can determine, for the group entity (e.g., the company “XYZ”), the email generation pattern 3970 to be the value of the LN field followed by a character “.” followed by an initial of the FN field followed by “@” and followed by “XYZ.COM.” Using the pattern 3970, the pattern generator 3950 can update the values of the values of the email fields of the member node profiles 3902 and 3912 to be “SMITH.J@XYZ.COM” and “XU.A@XYZ.COM,” respectively, and/or generate a value of the email field of the member node profile 3922 to be “JAMES.K@XYZ.COM.” Accordingly, the data processing system 9300 can update the respective company and domain name fields, which may result in increasing the confidence scores of the domain name fields.

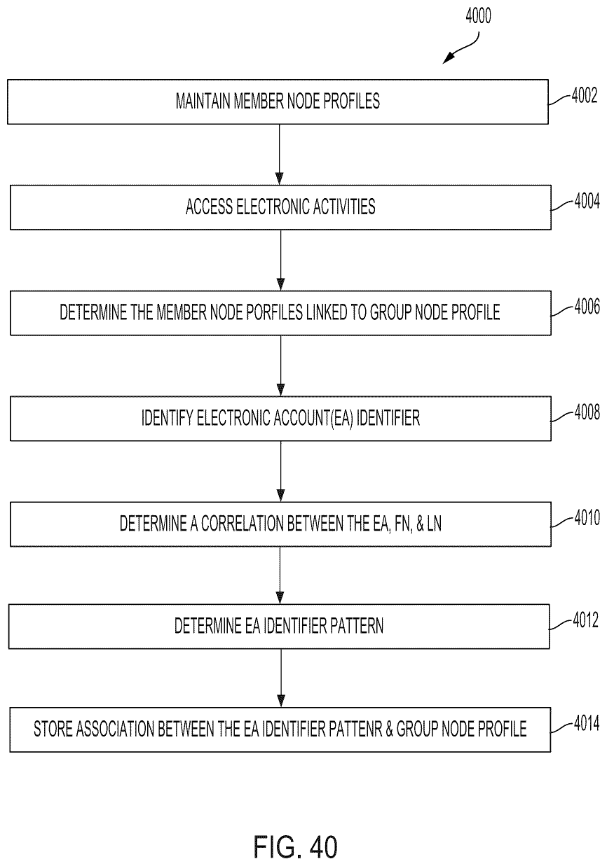

Referring now to FIG. 40, FIG. 40 illustrates a method 4000 for updating email addresses based on email generation patterns, in accordance with one or more embodiments. Operations of the method 4000 presented below are intended to be illustrative. In some embodiments, the method 4000 may be accomplished with one or more additional operations not described, and/or without one or more of the operations discussed. Additionally, the order in which the operations of the method 4000 as illustrated in FIG. 40 and described below is not intended to be limiting.

In some embodiments, the method 4000 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of the method 4000 in response to instructions stored electronically on an electronic storage medium. The one or more processing devices may include one or more devices configured through hardware, firmware, and/or software to be specifically designed for execution of one or more of the operations of the method 4000.

In brief overview, the method 4000 may include maintaining a plurality of member node profiles (BLOCK 4002). The method 4000 may include accessing a plurality of electronic activities (BLOCK 4004). The method 4000 may include determining the plurality of member node profiles linked to a group node profile (BLOCK 4006). The method 4000 may include identifying an electronic account identifier of each of the member node profiles (BLOCK 4008). The method 4000 may include determining a correlation between the electronic account identifier, a first name and a last name (BLOCK 4010). The method 4000 may include determining an electronic account identifier pattern (BLOCK 4012). The method 4000 may include storing an association between the electronic account identifier pattern and the group node profile (BLOCK 4014).

In further detail, the method 4000 may include maintaining a plurality of member node profiles (BLOCK 4002). As described herein, the data processing system 9300, or the node graph generation system 200, can construct a node graph based on electronic activities. The node graph can include a plurality of nodes (e.g., people, groups, or companies) and a plurality of edges between the nodes indicating activity or relationships that are derived from a plurality of data sources that can include one or more types of electronic activities. In some embodiments, each of the nodes, corresponding to an unique entity (e.g., a person, a group, a company, etc.), can be associated with a node profile. The data processing system 9300 can ingest, store, or otherwise mange such information as one or more node field-value pairs in each of the node profile. As such, each of the node profiles can include one or more node field-value pairs, where each node field-value pair of the node profile has a corresponding field and a node field value. It is appreciated that the node profiles can include different types of fields for different types of nodes. Member nodes and group nodes may have some common fields but may also include different fields. Further, member node profiles may include fields that get updated more frequently than group node profiles. Examples of some fields of member node profiles can include i) First name; ii) Last name; iii) Email; iv) job title; v) Phone; vi) Social media handle; vii) LinkedIn URL; viii) website; among others. Examples of some fields of group node profiles can include i) Company or Organization name; ii) Address of Company; iii) Phone; iv) Website; v) Social media handle; vi) LinkedIn handle; among others.

With specific reference to FIG. 39, the member node profile 3902 includes the email field with the corresponding value, “JSMITH@ACME.COM (at the first timestamp);” the first name (FN) field with the corresponding value, “JOHN;” the last name (LN) field with the corresponding value, “SMITH;” the company name field with the corresponding value, “ACME (at the first timestamp);” and the domain name field with the corresponding value, “ACME.COM (at the first timestamp).” Similarly, the member node profile 3912 includes the email field with the corresponding value, “AXU@ACME.COM (at the first timestamp);” the first name (FN) field with the corresponding value, “ABIGAIL;” the last name (LN) field with the corresponding value, “XU;” the company name field with the corresponding value, “ACME (at the first timestamp);” and the domain name field with the corresponding value, “ACME.COM (at the first timestamp).”

The method 4000 may include accessing a plurality of electronic activities (BLOCK 4004). In some embodiments, the data processing system 9300, or the node graph generation system 200, can ingest or access a plurality of electronic activities transmitted or received via electronic accounts associated with one or more data source providers. The data processing system 9300 can access electronic activities via one or more servers hosting or storing the electronic activities. The servers can store electronic activities transmitted from or received by accounts corresponding to an enterprise. For instance, the servers can be mail servers, phone log servers, calendar servers or any other entity that can store emails, calendar events, phone logs, or other electronic activities of accounts associated with an enterprise, such as a company. Referring to FIG. 25, the data processing system 9300 can access the first electronic activity 652a and the second electronic activity 652b. The first electronic activity 652a and the second electronic activity 652b can be accessed from a mail server of a first data source provider corresponding to the sender of the first electronic activity 652a and the recipient of the second electronic activity 652b. Since the second electronic activity 652b is a response to the first electronic activity 652a, the recipient of the second electronic activity 652b is the same person as the sender of the first electronic activity. In some embodiments, the first electronic activity 652a and the second electronic activity 652b can be accessed from one or more electronic activity accounts corresponding to the sender of the first electronic activity 652a or the sender of the second electronic activity 652b.

Responsive to ingesting the electronic activities, the data processing system 9300 can process the electronic activities. The data processing system 9300 can process the electronic activities by parsing the contents of the electronic activities and using the parsed electronic activities to link the electronic activities to one or more of the node profiles generated or maintained by the node profile manager 220. In some embodiments, the data processing system 9300 can update the one or more member node profiles using the parsed electronic activities.

In some embodiments, the data processing system 9300 can maintain a frequency distribution or count table. The frequency distribution or count table can be based on information included in the member node profiles maintained by the data processing system 9300. The data processing system 9300 can update the frequency distribution or count table responsive to ingestion of an electronic activity by the data processing system 9300.

The data processing system 9300 can maintain a first name distribution based on the count of each node value of the plurality of node field-value pairs that is associated with a first name field. The data processing system 9300 can maintain a last name distribution based on the count of each node value of the plurality of node field-value pairs that is associated with a last name field. In some embodiments, the data processing system 9300 can build a count table that includes first names, last names, and associated counts. For example, the FN field has associated values including “ARIANE,” “JOHN,” “ABIGAIL,” “KEVIN,” etc. The LN field has associated activity values including “XU,” “SMITH,” “JAMES,” “LEE,” etc. The activity field-value pair “FN: JOHN” has a count of 261, “FN: ABIGAIL” has a count of 7391, “LN: SMITH” has a count of 536, and “LN: XU” has a count of 3815.

As such, when an electronic activity including an email address “johnxu@examplemail.com” is identified, the data processing system 9300 can parse the email address to determine if the email address includes one or more names. In some embodiments, the data processing system 9300 can use the first name distribution or the last name distribution to generate the plurality of activity field-value pairs, such as activity field-value pairs for first name and last name. The data processing system 9300 can match portions of the string preceding the “@” character to the first name and last name values maintained by the data processing system 9300. Responsive to the data processing system 9300 identifying a match between a name and the string, the data processing system 9300 can determine a probability that the name is a first name or a last name based on the frequency count of the first name or the last name maintained in the data processing system 9300.

In the example above, the data processing system 9300 can determine a probability that johnxu corresponds to first name: JOHN and last name: XU. The data processing system 9300 can then attempt to match the electronic activity to a node profile using the node-value pairs, first name: John and last name: Xu in addition to the field value pair, email address: johnxu@examplemail.com. This technique can improve the system's ability to link electronic activities to the right node profiles and can improve the accuracy of the data processing system 9300.

The data processing system 9300 can determine a frequency score of an activity value of the plurality of activity field-value pairs. The data processing system 9300 can determine a first frequency score of a first activity value of the plurality of activity field-value pairs. The data processing system 9300 can determine a second frequency score of a second activity value of the plurality of activity field-value pairs. The data processing system 9300 can determine the first frequency score and the second frequency score based on a count of node values that match the activity values. In some embodiments, the data processing system 9300 can determine the frequency score based on the first name distribution or the last name distribution. The frequency score can be a representation of count. The frequency score can be the count divided by a factor to normalize the frequency score. For example, the frequency score of the value “JOHN” associated with the field “First Name” can have different frequency scores. The frequency score can be the count of “JOHN” associated with the field “First Name” divided by the total count of values associated with the field “First Name”. In another example, the frequency score can be the count of “JOHN” associated with the field “First Name” divided by the total count of values associated with the field “First Name” that were generated from electronic activities generated from the “ACME” system of record.

The method 4000 may include determining the plurality of member node profiles linked to a group node profile (BLOCK 4006). In some embodiments, the data processing system 9300 can determine that the plurality of member node profiles are linked to the group profile of a group entity based on the values of company name fields of the member node profiles. Upon identifying the values of the company name fields of the member node profiles, the data processing system 9300 can determine that the values of the company name field match a value of a company name field of the group node profile of the group entity or a domain name field of the group node profile. For example in FIG. 39, the data processing system 9300 can determine that the member node profile 3902 is linked to the group profile of a group entity (e.g., a company ACME) by determining that the company name field of the member node profile 3902 matches the value of a company name field of a group node profile, belonging to the group entity “ACME,” or the value of a domain name field of the group node profile. Similarly, the data processing system 9300 can determine that the member node profile 3912 is linked to the group profile of the group entity (e.g., the company ACME) by determining that the company name field of the member node profile 3912 matches the value of the company name field of the group node profile, belonging to the group entity “ACME,” or the value of the domain name field of the group node profile.

The method 4000 may include identifying an electronic account identifier of each of the member node profiles (BLOCK 4008). In some embodiments, the data processing system 9300 can identify, for each of the plurality of member node profiles, a field-value pair of the member node profile corresponding to an electronic account identifier of the member node profile. The electronic account identifier can include an address via which electronic activities are transmitted from or received by the electronic account of the member node profile, e.g., the value of the email field of each of the member node profiles. In the context of FIG. 39, the data processing system 9300 can determine the value of the email field 3903 of the member node profile 3902 to be “JSMITH@ACME.COM,” and the value of the email field 3913 of the member node profile 3912 to be “AXU@ACME.COM.”

In some embodiments, the data processing system 9300 can determine that the group entity includes a plurality of different domain names based on a second, different value of the email field of one or more of the member node profiles. The data processing system 9300 can identify, for each of the plurality of member node profiles, a second value of the email field. The second email value can indicate a second, different domain name. The data processing system 9300 can count the number of member node profiles that include the second, different domain name in the respective second value of the email field. The data processing system 9300 can compare the count with a threshold value. In response to determining that the count satisfies the threshold value (e.g., greater than the threshold value), the data processing system 9300 can determine that the group entity also owns the second, different domain name. Accordingly, the data processing system 9300 can store the association between the second, different domain name and the group entity.

The method 4000 may include determining a correlation between the electronic account identifier, a first name and a last name (BLOCK 4010). In some embodiments, concurrently with or subsequently to identifying the email address of each of the member node profiles, the data processing system 9300 can further identify a second field-value pair corresponding to a first name of the member node profile and a third field-value pair corresponding to a last name of the member node profile. Upon identifying the respective values of the first name field and the last name field, the data processing system 9300 can extract a character string preceding a predetermined character of the value of the email field. In some embodiments, the data processing system 9300 can determine that the character string includes at least one of a first name or a last name based on the respective probability scores. The data processing system 9300 can compare the character string to the values of the first name field and last name field. Based on the comparison, the data processing system 9300 can determine, for each member node profile, a correlation between the email address, first name, and last name.

In the context of FIG. 39, the data processing system 9300 can extract, for the node profile 3902, the character string preceding the character “@” to be “JSMITH.” Based on the values of the FN field and LN field of the node profile 3902 (“JOHN” and “SMITH,” respectively), the data processing system 9300 can determine that the character string “JSMITH” is composed of an initial of a name “JOHN,” which is with a relatively high probability score to be a first name, and followed by a name “SMITH,.” which is with a relatively high probability score to be a last name. Similarly, for the node profile 3912, the data processing system 9300 can determine that the character string “AXU” is composed of an initial of a name “ABIGAIL,” which is with a relatively high probability score to be a first name, and followed by a name “XU,.” which is with a relatively high probability score to be a last name. As such, the data processing system 9300 may determine the correlation as the email address being composed of the initial of the value of a FN field followed by the value of a LN field, which can be used to determine an email generation pattern.

The method 4000 may include determining an electronic account identifier pattern (BLOCK 4012). In some embodiments, based on the determined correlation, the data processing system 9300 can determine the electronic account identifier pattern, e.g., an email generation pattern, used by the group entity to generate email addresses. The data processing system 9300 can combine the determined correlation with the value of the domain name field of the member node profile or the group node profile. Continuing with the above example in FIG. 39, the data processing system 9300 can determine the email generation pattern 3960 as “FN_INITIAL+LN @ACME.COM.” In some embodiments, the data processing system 9300 can use the email generation pattern 3960 and the values of FN field and LN field of the node profile 3922 to generate a value of the email field of the node profile 3922.

Using the email generation pattern, the data processing system 9300 can match a new electronic activity to an existing member node profile. In some embodiments, the data processing system 9300 can access the new electronic activity to identify a new email address by parsing the new electronic activity. The email address may be an email address of the sender or recipient of the new electronic activity. Based on the identified email address, the data processing system 9300 can determine whether the group entity generated this email address by comparing a domain name of the email address (e.g., a character string following the “@” character) with the value of the domain name field of the group node profile. In response to determining that the new email address corresponds to the group entity, the data processing system 9300 can use the email generation pattern to infer or otherwise determine at least one of a first name or a last name of an entity associated with the new email address. For example, the data processing system 9300 may determine a new email address to be “DHOLT@ACME.COM.” Based on the email generation pattern (e.g., 3960), the data processing system 9300 can determine that the entity associated with the new email address has a last name, “HOLT,” and a first name with an initial “D.” Accordingly, the data processing system 9300 can generate a first activity field-value pair identifying the new email address, and a second activity field-value pair identifying the first name or last name determined from the email address. In response to determining that the value of a FN field of one of the plurality of member node profiles matches the determined first name or the value of a LN field of one of the plurality of member node profiles matches the determined last name, the data processing system 9300 can match the new email to the member node profile.

Using the email generation pattern on the value of an email field of a member node profile, the data processing system 9300 can infer or otherwise generate, for the member node profile, at least one of a value of the FN field or a value of the LN field. In some embodiments, the data processing system 9300 can store a value of the email field of the member node profile (e.g., an email address) but lack the values for the FN field and LN field. Based on the email generation pattern, the data processing system 9300 can determine at least one of a first name or last name from the email address. Accordingly, the data processing system 9300 can store the first name or last name as the value of the corresponding FN or LN field.

In some embodiments, the data processing system 9300 can determine a relationship between a first group entity and a second group entity. The data processing system 9300 can determine, for each of a plurality of member node profiles of the first group entity, that a first confidence score of a respective value including a first domain name of the member node profile is decreasing, for example in FIG. 39, the confidence scores of the domain field-value pair, ACME.COM, of the member node profile 3902 and the domain field-value pair, ACME.COM, of the member node profile 3912. Subsequently, the data processing system 9300 can determine, for each of the plurality of member node profiles of the first group entity, that a second confidence score of a respective value including a second domain name corresponding to the second group entity of the member node profile is increasing, for example in FIG. 39, the confidence scores of the domain field-value pair, XYZ.COM, of the member node profile 3902 and the domain field-value pair, XYZ.COM, of the member node profile 3912. Responsive to determining such decreasing first confidence score and increasing second confidence score, the data processing system 9300 can determine the relationship between the first domain name corresponding to the first group entity and the second domain name corresponding to the second group entity. For example, the data processing system 9300 may determine that the company XYZ has already acquired (owned) the company ACME.

With specific reference to FIG. 39, upon determining the relationship between the companies XYZ and ACME, the data processing system 9300 can update the values of the email field of the node profile 3902 and the email field of the node profile 3912 based on the email generation pattern 3970. As described above, the data processing system 9300 can determine the email generation pattern 3970 to be “LN.FN_INITIAL@XYZ.COM” based on determining a correlation between LEE.A@XYZ.COM (the value of the email field of the node profile 3932), ALICE (the value of the FN field of the node profile 3932), and LEE (the value of the LN field of the node profile 3932). In response to determining that the company ACME is now owned (acquired) by the company XYZ, the data processing system 9300 can update the value of the email field of the node profile 3902 to be “SMITH.J@XYZ.COM,” and the value of the email field of the node profile 3922 to be “XU.A@XYZ.COM.”

In some embodiments, the data processing system 9300 can determine such an acquisition based on detecting a sufficient amount of bounceback emails. The data processing system 9300 can determine that one or more emails corresponding to email addresses generated by a first group entity have triggered generation of a number of bounceback emails within a predetermined time period. According to some embodiments, the data processing system 9300 can determine such a time period based on a timestamp of the acquisition. The data processing system 9300 can determine a second group entity (e.g., XYZ) from content included in the bounceback emails. For example, some of the bounceback emails can include a message “WELCOM TO XYZ.” The data processing system 9300 can determine that the number of bounceback emails satisfies an entity change detection policy. The entity change detection policy may include a threshold number of bounceback emails. Once the detected number of bounceback emails exceeding the threshold, the data processing system 9300 can determine that the first group entity has acquired by the second group entity.

The method 4000 may include storing an association between the electronic account identifier pattern and the group node profile (BLOCK 4014). For example, the data processing system 9300 can store, in a data structure, an association between the email generation pattern(s) and the group node profile. Referring to FIG. 39, the data processing system 9300 can store an association between email generation pattern 3960 and the group node profile associated with the company ACME, and an association between the email generation pattern 3970 and the group node profile associated with the company XYZ.

### 26. Determining a Preferred Communication Channel Based on Determining a Status of a Node Profile Using Electronic Activities

The systems and methods disclosed herein, including those described in detail below, provide for, amongst other things, determining a preferred communication channel based on determining a status of a node profile using electronic activities. By determining a status of a node profile, for example, times when a user is available and preferred modes of communication, the system and methods can determine specific times to send a notification. Additionally, system can use the status of a node profile to determine preferred modes of communication with a user of a client device. A user may experience message fatigue if communications are not appropriately timed, the user may be unable to receive messages through certain modes of communication, or the user may prefer specific modes of communication at given times. The systems and methods provided herein can dynamically, and in real-time, provide notifications and reminders through a preferred communication channel.

According to some embodiments, a data processing system may access a plurality of electronic activities transmitted or received via electronic accounts associated with data source providers. The data processing system may maintain a plurality of node profiles corresponding to a plurality of unique entities. Each node profile can include one or more node field-value pairs. The data processing system may identify a node profile from the plurality of node profiles for the at least one participant of each of the plurality of electronic activities. The data processing system may generate, for anode profile, based on a subset of the plurality of electronic activities linked to the node profile, a communication mode schedule. The communication mode schedule can identify one or more communication modes over which to communicate with a first entity corresponding to the node profile at one or more time periods. The data processing system may transmit a first notification to a first device of the first entity via a communication mode selected using the communication mode schedule.

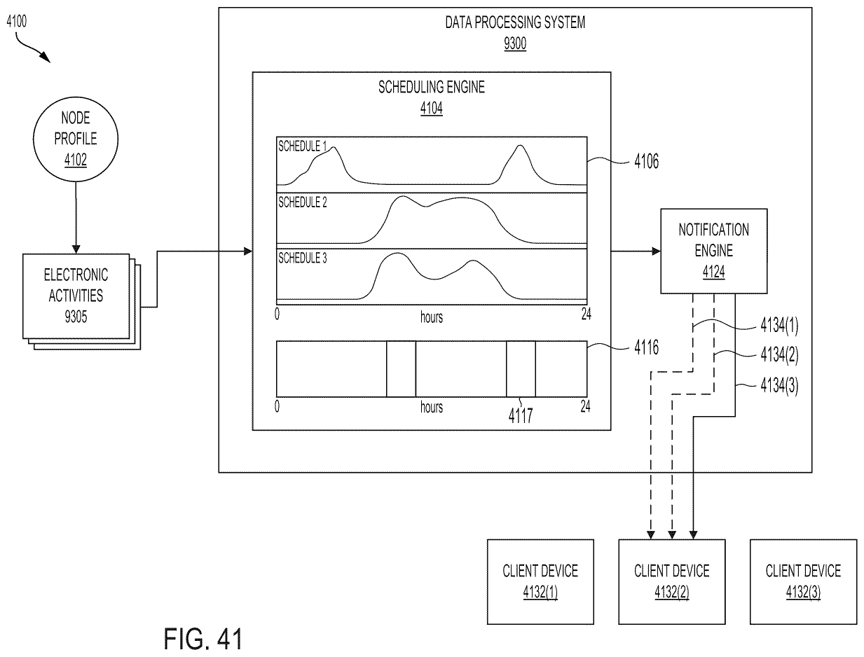

FIG. 41 illustrates a workflow 4100 for selecting a communication channel based on determining a status of a node profile using electronic activities. The data processing system 9300 can identify a node profile 4102. From the node profile 4102, the data processing system 9300 can identify a plurality of electronic activities 9305 linked or associated with the node profile 4102. The data processing system 9300 can include a scheduling engine 4104. The scheduling engine 4104 can generate a communication mode schedule 4106 from the plurality of electronic activities 9305 linked to the node profile 4102. The scheduling engine 4104 can generate an availability schedule 4116 based on the plurality of electronic activities 9305. The data processing system 9300 can include a notification engine 4124 that can use the communication mode schedule 4106 and the availability schedule 4116 to select a client device 4132 to which to send a notification and a communication channel 4134 over which to transmit the notification.

The data processing system 9300 can identify a node profile 4102 from a plurality of node profiles. The plurality of node profiles can be associated with an opportunity record object. An opportunity record objects can be associated with one or more node profiles. For example, the opportunity record object can be linked with one or more contact record objects, which can include field-value pairs that can be used to identify a node profile. The data processing system 9300 can identify a node profile 4102 of the one or more node profiles linked with the opportunity record object. The data processing system 9300 can identify a node profile 4102 that is a main contact for the opportunity record object. The data processing system 9300 can identify a node profile 4102 from a system of record. The data processing system 9300 can identify the node profile 4102 in response to identifying that the opportunity (or other) record object includes an action to be taken or a notification to be delivered. The node profile 4102 can be associated with the user identified to receive the notification or perform the action.

The data processing system 9300 can access a plurality of electronic activities 9305 linked to or otherwise associated with the identified node profile 4102. The data processing system 9300 can access a plurality of electronic activities 9305 transmitted or received via electronic accounts of one or more data source providers 9350. As described above, each data source provider 9350 can include one or more data sources 9355a-n and/or one or more system of record instances 9360. Examples of data sources can include electronic mail servers, telephone log servers, contact servers, other types of servers (e.g., instant message or communication servers) and end-user applications that may receive or maintain electronic activity data. In some embodiments, the data sources 9355 provide (e.g., transmit or allow access to) the electronic activities 9305 to an aggregator system (e.g. an aggregator server), and the aggregator provides at least a portion of the electronic activities 9305 to the data processing system 9300. The aggregator system may detect duplicate electronic activities 9305 (e.g., from a first data source 9355a with access to a sender's electronic activity 9305, and from a second data source 9355b with access to a receiver' electronic activity 9305), and may process the duplicates appropriately (e.g., by flagging the duplicates as such, or by deleting one of the duplicates, or by refraining from providing one of the duplicates to the data processing system 9300). In some embodiments, the data sources 9355 provide the electronic activities 9305 without such aggregation of electronic activities 9305 from various data sources 9355.

Accessing the electronic activities 9305 can be performed as part of ingesting the electronic activities 9305 in any manner described herein. As described above, the electronic activities 9305 may be accessed to attempt to match the electronic activities 9305 to record objects 1602 and/or to update existing matches between electronic activities 9305 and record objects 1602. The plurality of electronic activities 9305 may be stored in a buffer or in a queue (e.g., in each individual data source 9355, or in the aggregator system), or may be accessed according to some ordering policy. In some implementations, the plurality of electronic activities 9305 can be accessed in chronological order (e.g., based on a time stamp of the electronic activity, or based on a time the electronic activity was transmitted to or retrieved by the data processing system 9300). In some implementations, the plurality of electronic activities 9305 are accessed in an order based on a priority of the electronic activities 9305 (e.g., from highest priority to lowest priority). The priority may be determined by the data processing system 9300 based on a priority flag or tag for the electronic activities 9305, or based on another priority policy implemented by the data processing system 9300.

The data processing system 9300 can parse the plurality of electronic activities 9305. The data processing system 9300 can parse the contents of the plurality of electronic activities 9305. For example, the data processing system 9300 parse the header, footer, or body of an electronic activity 9305. The data processing system 9300 can identify an electronic activity type from the parsed electronic activities 9305. The plurality of electronic activities 9305 can include an electronic activity type. Examples of electronic activity types can include emails, text messages, calendar invitations, among others. The data processing system 9300 can identify a communication channel 4134 from the parsed electronic activities 9305. A communication channel 4134 can include a mode of communication. The mode of communication can be based on the type of the electronic activity. For example, a mode of communication can include a phone call, a text message, an email, or an instant message. In some implementations, the communication mode can identify a specific protocol, such as Post Office Protocol (POP) or Exchange for emails. The communication channel 4134 can be a mode of communication by which the data processing system 9300 communicates with a client device 4132. The data processing system 9300 can identify a time from the parsed electronic activities 9305. The time of the electronic activity can include the time at which the electronic activity was sent by the sender of the electronic activity or received by the recipient of the electronic activity (or received by the data processing system 9300).

The data processing system 9300 can include a scheduling engine 4104. The scheduling engine 4104 can generate a communication mode schedule 4106 from the plurality of electronic activities 9305 (or data parsed therefrom). For example, the scheduling engine 4104 can generate a communication mode schedule 4106 for a user associated with the node profile 4102. The scheduling engine 4104 can access the plurality of electronic activities 9305 (or data parsed therefrom) to determine times throughout the day during which the user is active. For example, the scheduling engine 4104 can determine the volume of emails a user sends over a 24-hour period. In some implementations, the communication mode schedule 4106 can include scheduling information for each of the different types of electronic activities 9305. For example, the scheduling engine 4104 can parse the electronic activities 9305 into different types. For example, as illustrated in FIG. 41, the communication mode schedule 4106 can include schedules for three different electronic activity types. In the example illustrated in FIG. 41, the scheduling engine 4104 can use the timestamps parsed from the electronic activities 9305 to determine the times at which the user made or received phone calls (SCHEDULE 1), sent or received emails (SCHEDULE 2), or sent or receive IM messages (SCHEDULE 3) over a 24-hour period. The scheduling engine 4104 can generate a communication mode schedule 4106 over various periods of time. For example, the scheduling engine 4104 can generate a communication mode schedule 4106 over a 24-hour period, over a work-week (M-F), over a year, or over multiple years. In this way, the scheduling engine 4104 can derive trends and make predictions as to times during which a user is likely to send or receive electronic activities.

The communication mode schedule 4106 can be based on time at which electronic activities are transmitted, received, or both transmitted and received. For example, the communication mode schedule 4106 for phone call can indicate times at which the user received and made phone calls or the scheduling engine 4104 can generate a first schedule indicating when phone calls were made and a second schedule indicating when phone calls were received. The scheduling engine 4104 can generate the communication mode schedule 4106 by generating a distribution or histogram of the times at which the electronic activities are sent or received. For example, the scheduling engine 4104 can bin the 24 hour day into 24 1-hour bins and count the number of electronic activities received or transmitted (by the user associated with the node profile) within each of the bins.

The communication mode schedule 4106 can include different levels of activity or schedules for various mode of communication. For example, the communication mode schedule 4106 can include levels of activity of a user with respect to the user's emails, phone calls, and text messages. For example, the communication mode schedule 4106 can include information pertaining to the number of emails a user sends over a given period of time. The communication mode schedule 4106 can include information pertaining to the length of emails a user sends over a given period of time. The communication mode schedule 4106 can include information pertaining to the number of text messages a user sends over a given period of time. The communication mode schedule 4106 can include information pertaining to the length of text messages a user sends over a given period of time. The communication mode schedule 4106 can include information pertaining to the number of phone calls a user sends over a given period of time. The communication mode schedule 4106 can include information pertaining to the length of phone calls a user sends over a given period of time.

The scheduling engine 4104 can use historical data to generate the communication mode schedule 4106. The scheduling engine 4104 can identify a plurality of electronic activities linked to the first node profile. The scheduling engine 4104 can identify, from the plurality of electronic activities, a response rate for each type of electronic activity. The scheduling engine 4104 can use the response rate to select a communication mode channel via which to communicate. For example, the scheduling engine 4104 can select a communication mode with the highest response rate. The data processing system 9300 can communicate with the user via the communication mode likely to elicit a response or action from the user. In some implementations, the response rate can indicate the time delay between when the user receives an electronic activity and the time when the user transmits an electronic activity in response to the received electronic activity. In some implementations, the response rate can indicate a ratio or percentage of times that the user responds to an electronic activity of a given type. For example, the response rate may indicate that the user responds to 94% of emails and 46% of calls.

The scheduling engine 4104 can generate an availability schedule 4116. The availability schedule 4116 can include information pertaining to the availability of the user associated with the node profile. The scheduling engine 4104 can generate the availability schedule 4116 from the electronic activities. For example, the scheduling engine 4104 can generate the availability schedule 4116 from calendared appointments or other electronic activities that may indicate the availability of the user through one or more modes of communication. For example, if the user has a meeting between 10:30 am and 11:30 am, the availability schedule 4116 can show that the user is unavailable from 10:30 am and 11:30 am. In another example, the scheduling engine 4104 can parse an electronic activity to identify that the electronic activity is a flight confirmation email. The scheduling engine 4104 can indicate via the availability schedule 4116 that the user is available via email but unavailable via phone calls during the duration of the flight. The user can also set aside a time on the user's calendar to attend to emails by blocking off a time period from 8:00 am to 9:00 am. While the user's calendar can show that the user is unavailable, the availability schedule 4116 can show that the user is actually available to send emails, based upon the user's sending of emails between 8:00 am and 9:00 am. The scheduling engine 4104 can generate the availability schedule 4116 from an instant messaging client or other client that provides status information. For example, the instant messaging client (e.g., Skype for Business) can show that a user is available based on computer activity. The availability schedule 4116 be a live schedule of availability that shows if a user is available or unavailable. In some implementations, the availability schedule 4116 can indicate that the user is available by a communication mode when the user is actively sending (or has sent within a given time window) an electronic activity via the communication mode.

The system 4100 can include a notification engine 4124. The notification engine 4124 can access the communication mode schedule 4106 generated by the scheduling engine 4104 to select times for transmitting a notification to a client device 4132. For example, the notification engine 4124 can send a notification along a communication channel 4134. The notification engine 4124 can select a communication channel 4134 from among a plurality of communication channels 4134. For example, the plurality of communication channels 4134 can include communication channel 4134(1): phone call, communication channel 4134(2): text message, or communication channel 4134(3): email. Each of the communication channels 4134 can correspond a different one of the schedules in the communication mode schedule 4106. The notification engine 4124 can send a notification though the communication channel 4134(3): email based on the user's availability schedule and communication mode schedule 4106. For example, the notification engine 4124 can determine that the user is available and for the current time the email schedule of the communication mode schedule 4106 has the highest distribution and thereby select the communication channel 4134(3): email. In this way, the user can receive an email to follow up with a buyer.

The notification engine 4124 can select a client device 4132 to which to send the notification. For example, the user may be associated with a plurality of client devices (e.g., cell phone, laptop, or tablet). Each of the client devices can include a plurality of communication channels for communication with the data processing system 9300. The notification engine 4124 may select from client device 4132(1): cell phone, client device 4132(2): laptop, client device 4132(3): tablet. The data processing system 9300 can identify an action to be performed. The data processing system 9300 can identify a recipient related to the action. The notification engine 4124 can select the client device 4132 based on the action to be performed. The notification engine 4124 can select the client device 4132 based on the recipient related to the action. For example, the notification engine 4124 can select a client device 4132(1): cell phone based on the action to be performed being a phone call.

In brief overview, the method 4200 may include accessing electronic activities (BLOCK 4202). The method 4200 may include maintaining node profiles (BLOCK 4204). The method 4200 may include identifying a node profile (BLOCK 4206). The method 4200 may include generating a communication mode schedule (BLOCK 4208). The method 4200 may include transmitting a notification (BLOCK 4210).

In further detail, the method 4200 may include accessing electronic activities (BLOCK 4202). The data processing system 9300 can access a plurality of electronic activities transmitted or received via electronic accounts. The electronic accounts can be associate with one or more data providers. The data processing system 9300 can access a plurality of electronic activities 9305 transmitted or received via electronic accounts of one or more data source providers 9350. Each data source provider 9350 can include one or more data sources 9355a-n and/or one or more system of record instances 9360. Examples of data sources can include electronic mail servers, telephone log servers, contact servers, other types of servers (e.g., instant message or communication servers) and end-user applications that may receive or maintain electronic activity data. In some embodiments, the data sources 9355 provide (e.g., transmit or allow access to) the electronic activities 9305 to an aggregator system (e.g. an aggregator server), and the aggregator provides at least a portion of the electronic activities 9305 to the data processing system 9300. The aggregator system may detect duplicate electronic activities 9305 (e.g., from a first data source 9355a with access to a sender's electronic activity 9305, and from a second data source 9355b with access to a receiver' electronic activity 9305), and may process the duplicates appropriately (e.g., by flagging the duplicates as such, or by deleting one of the duplicates, or by refraining from providing one of the duplicates to the data processing system 9300). In some embodiments, the data sources 9355 provide the electronic activities 9305 without such aggregation of electronic activities 9305 from various data sources 9355.

The method 4200 may include maintaining node profiles (BLOCK 4204). The data processing system 9300 can maintain a plurality of node profiles. The node profiles can correspond to a plurality of unique entities, such as users, contacts, or companies. Each node profile can include one or more node field-value pairs. Each node profile includes one or more field-value pairs associating a node field value to a corresponding field of the node profile. For example, the data processing system 9300 can maintain a plurality of node profiles that can include information such as first name, last name, company, department and job title, each of which are represented by fields having one or more values, each value having a confidence score assigned to the value. The data processing system 9300 is configured to update the plurality of node profiles using the plurality of record objects.

The method 4200 may include identifying a node profile (BLOCK 4206). The data processing system 9300 can identify a node profile from the plurality of node profiles for the at least one participant of each of the plurality of electronic activities. The data processing system 9300 can identify a particular node profile of a participant of each of the plurality of electronic activities. The node profile can identify a main contact (e.g., owner) for an opportunity record object. The node profile can be a seller associated with the opportunity record object. The data processing system 9300 can identify the node profile responsive to determining that an action should be taken on an opportunity associated with the record object.

The method 4200 may include generating a communication mode schedule (BLOCK 4208). The data processing system 9300 can generate a communication mode schedule identifying one or more communication modes over which to communicate with a first entity corresponding to the node profile at one or more time periods. The data processing system 9300 can generate the communication node schedule for a node profile. The data processing system 9300 can generate the communication node schedule based on a subset of the plurality of electronic activities linked to the node profile. For example, the subset can include electronic activities transmitted by the entity associated with the node profile or the electronic activities received by the entity associated with the node profile. The communication mode schedule can indicate, for each mode of communication, the time distribution of historical electronic activities. For example, the communication mode schedule can include a count within each of 24, 1-hour time bins to indicate during which time of day the user sends or receives more electronic activities.

The data processing system 9300 can generate the first notification. The first notification can identify an action to be performed and a recipient related to the action. The first notification can include an action that should be performed and the recipient of the communication. For example, the data processing system 9300 can determine that an email should be sent to a first entity. The first entity can be the user associated with the node profile. The data processing system can generate a communication mode schedule for the node profile.

The data processing system 9300 can use historical data to generate the communication mode schedule. The historical data can be or can include previously transmitted or received electronic activities or data derived therefrom. For example, the data processing system 9300 can identify a plurality of electronic activities linked to the first node profile. The data processing system 9300 can identify a time at which each of the electronic activities were transmitted or received. In some implementations the data processing system 9300 can identify, from the plurality of electronic activities, a response rate for each type of electronic activity. The data processing system 9300 can generate a communication mode schedule based on the time at which each of the electronic activities were transmitted or received. The communication mode schedule can be based on the response rate for each type of electronic activity. The communication mode schedule can indicate, for a given time during the day, over which mode of communication the user associated with the node profile is likely to be most responsive. The data processing system 9300 may determine that the user is most likely to response over a communication mode with the highest distribution at the current time. For example, and referring to FIG. 41, the communication mode schedule 4106 indicates that for the communication mode illustrated in the SCHEDULE 1, the distribution is highest in the morning and in the early evening while the SCHEDULE 2 and SCHEDULE 3 have a higher relative distribution during the middle of the day.

The data processing system 9300 can use the communication mode schedule to select a communication mode to transmitting the notification to one of the user's client devices. For example, the data processing system 9300 can select a communication mode with the highest response rate or distribution. For example, and referring to FIG. 41, during the morning hours, the notification engine 4124 may select the communication mode associated with the SCHEDULE 1 because SCHEDULE 1 has a higher distribution during the morning hours when compared to SCHEDULE 2 and SCHEDULE 3. In some implementations, the notification engine 4124 can select the communication mode based on both the communication mode schedule 4106 and the availability schedule 4116. For example, the availability schedule 4116 can include a time period 4117 when the user is not available by one or more of the communication modes. For example, as illustrated in FIG. 41, during the evening hours, the availability schedule 4116 can indicate that the user has a meeting and is unavailable. In this instance, the notification engine 4124 may select to not send the notification via the communication mode associated with SCHEDULE 1 because the availability schedule 4116 indicates that the user is unavailable during the evening hours. When the availability schedule 4116 indicates that the user is unavailable, rather than selecting the communication mode with the highest distribution at the current time, the notification engine 4124 may select a communication mode that is passive even though the communication mode has a lower distribution for the current time. For example, emails or instant messages can be considered passive communication modes because the user can receive the notification without taking action. A phone call can be considered an active communication mode because the user cannot receive the notification without actively answering the call.

In some implementations, the notification can involve a second entity (or a recipient of the action identified in the notification). The second entity can be the intended recipient of an action identified in the notification. For example, the notification can instruct the recipient of the notification to transmit an email to the second entity. The data processing system 9300 can determine a second communication mode schedule of the second entity based on electronic activities linked with a node profile of the second entity. The data processing system 9300 can repeat the process described above, but for the electronic activities linked with the node profile the second entity, to generate a communication mode schedule and an availability schedule for the second entity. The data processing system 9300 can identify, for a time period, a communication mode via which to communicate with the second entity. The data processing system 9300 can select an electronic activity type of the electronic activity based on the communication mode via which to communicate with the second entity. The data processing system 9300 can identify the electronic activity type with which to communicate with the second entity in the first notification. For example, the data processing system 9300 can determine that an email notification should be sent to the first entity and include an action to be performed, sending a reminder to a second entity. The email notification to the first entity can indicate that the preferred communication mode of the second entity, at the given time, is via a phone call.

In some embodiments, the data processing system 9300 can identify a plurality of electronic activities linked to the first node profile. The data processing system 9300 can identify, for each electronic activity of the plurality of electronic activities, an electronic activity type. The data processing system 9300 can generate an availability schedule of the first node profile. The data processing system 9300 can generate the availability schedule based on the plurality of electronic activities. The availability schedule can indicate time frames that a user of the first device is available. The data processing system 9300 can generate a communication mode schedule for the user based on historical electronic activities. The data processing system 9300 can override the communication mode schedule based on the availability schedule. For example, the data processing system 9300 can determine that the communication mode schedule indicates that the user is unavailable during a certain period of time. In some implementations, the availability schedule can indicate that the entity is currently active via one of the communication modes. For example, the availability schedule can indicate that the user is actively sending messages via an instant messaging platform (an example communication mode). In this way, the data processing system 9300 can override the communication mode schedule based on the availability schedule. For example, for the given time, the messaging platform may not have the highest distribution in the communication mode schedule; however, the notification engine can select the messaging platform as the communication mode because the user is actively using the messaging platform. In another example, the data processing system 9300 can determine that the preferred way to contact a user is to contact the user at 10 am on Tuesday. The data processing system 9300 can identify that the user has a meeting at 10 am on Tuesday. Instead of calling the user at 10 am on Tuesday, the data processing system 9300 an send an email to the user so as to avoid interrupting the user at that time.

The method 4200 may include transmitting a notification (BLOCK 4210). The data processing system 9300 can transmit a first notification to a first device of the first entity via a communication mode selected using the communication mode schedule. The data processing system 9300 can send the first notification to the first device of the first entity via a preferred mode. For example, the data processing system 9300 can send the first notification via an email and not by phone. In a situation where the first entity is unreachable by phone (e.g., on a plane) the data processing system 9300 can determine that the preferred communication channel to send the notification is by email.

In some embodiments, the data processing system 9300 can select a device to which to send the notification. The data processing system 9300 can select from a plurality of devices including the first device, the first device to which to transmit the notification. The data processing system 9300 can make the selection based on the communication mode schedule of the first node profile. For example, the user may have multiple devices to which the data processing system 9300 can send the notification. The data processing system 9300 can determine which device to send the notification at a particular time. For example, the communication mode schedule 4106 can include schedules for each of the user's client devices. For example, and referring to FIG. 41, the user can have three client devices. Each of the three client devices can communication with the notification engine 4124 via three modes of communication. The scheduling engine 4104 can generate a communication mode schedule 4106 that can include 9 schedules (e.g., 3 schedules for each of the three client devices).

In some embodiments, the data processing system 9300 can determine the preferred communication mode for transmitting the notification based on the node profile data. The data processing system 9300 can identify a node profile of the second entity. The data processing system 9300 can identify a plurality of electronic activities linked to the node profile of the second entity. The data processing system 9300 can determine, for each of a plurality of electronic activity types, a response rate based on the plurality of electronic activities. The data processing system 9300 can select a second communication mode for transmission of a second notification to the second device based on the response rate of each of the plurality of electronic activity types.

In some embodiments, the data processing system 9300 can select the communication mode based on the action to be performed in the notification. The data processing system 9300 can identify a record object of a plurality of record objects of one or more systems of record. Each of the plurality of record objects can include one or more object fields having one or more field value pairs. The data processing system 9300 can determine an action to perform to increase a completion score of the record object. The data processing system 9300 can select the communication mode based on the action to be performed to increase the completion score of the record object.

### 27. Inferring a Time Zone of a Node Profile Using Electronic Activities

The present disclosure relates to systems and methods for inferring a time zone of a node profile using electronic activities. An enterprise, company, or other entity may employ people in disparate geographic areas. For example, while an entity may have a headquarters located in a particular city or region, there may be other offices for the entity in different areas of the country or international offices in other parts of the world. Some employees may also work remotely, either permanently or temporarily. In addition, employees may travel between offices or may travel to meet clients or prospects in different locations or travel for pleasure. As a result, it can be challenging to determine a person's time zone during a specified time period, even if other information is available for the person, such as a work address of the person, the location of a headquarters or primary office for the person's employer.

This disclosure describes techniques for inferring a person's time zone based on the electronic activities the person sends during a specified time period. For example, during a typical day, a person may send and receive a number of emails, phone calls, or other electronic activities as a part of their employment. In some instances, a person may be more likely to send and receive a larger volume of electronic activities during traditional working hours, such as during the morning, afternoon, and early evening, and may be less likely to send and receive electronic communications outside of traditional working hours, such as overnight. The systems and methods described herein can access electronic activities generated by a person within a specified time period, and can use the electronic activities to determine the person's schedule and predict the time zone in which the person was located during the time period. In some implementations, the systems and methods described herein can generate a temporal distribution of electronic activity for time intervals within the time period. The systems and methods described herein determine patterns based on the temporal distribution to infer the person's time zone during the time period for which the electronic activities were accessed.

As one example, a user, such as an employee, may initiate, transmit, receive, respond to, or otherwise participate in electronic activities over a specified period of time (e.g., from 9:00A-5:00P in the time zone in which the employer's headquarters is located). Another user who works at the same employee may participate in electronic activities over a second period of time (e.g., from 1:00P-9:00P in the time zone in which the employer's headquarters is located). A data processing system may identify electronic activities engaged in by the users. For each user, the system may generate a temporal distribution of electronic activities for a time period (such as a day, a week, a month, or any other time period). The system may determine an electronic activity pattern for each user based on the temporal distributions of electronic activities for each user during the time period. The system may correlate the temporal distributions for each user with a respective time zone. For example, the system may determine that the first person is located in the time zone of the headquarters of the employer, while the second user is in a location that is time shifted from the time zone of the headquarters of the employer, based on electronic activity patterns for the users.

In some implementations, the system can use the inferred time zone information to support or corroborate other types or forms of information. For example, a system of record may store information relating to, relevant to, or correlated with time zone information. In some implementations, the system of record can maintain field-value pairs for record objects. A field-value pair may relate to a location of a user associated with a record object. The system can use the time zone information to support or corroborate such location information. For example, some systems of record may record a value of a location field using a company headquarters location (e.g., city) as a default value for record objects associated with all employees of the company, even though some employees may work remotely or may work in offices that are not at the headquarters location.

Determining or inferring a time zone for a person based on the person's electronic activities can help to support such location information. In some implementations, the systems and methods disclosed herein can increase or decrease a confidence score associated with a field-value pair of a record object or node profile, based on inferred time zone information. For example, if a person's electronic activities indicate that the person is in a time zone that does not correspond to a stored location value for the person, the systems and methods of this disclosure can reduce a confidence score associated with the stored location value. In some implementations, if a person's electronic activities indicate that the person is in the same time zone as a stored location value for the person, the systems and methods of this disclosure can increase the confidence score associated with the stored location value. In some implementations, the systems and methods of this disclosure can update one or more values of field-value pairs, such as a location field-value pair or a time zone field-value pair, based on the inferred time zone information.

In some implementations, the system can determine the electronic activity pattern for the user based on electronic activities over a large period of time. For instance, the system may rely on activities over a 90 day period of time. Doing so can help the system determine days that the user may be on vacation, traveling, or otherwise outside of the user's usual time zone. In addition, the system can use the inferred time zone information to disambiguate between two or more users who may share other characteristics (e.g., first name and last name) in common with one another. For example, when the system accesses a new electronic activity that could be attributed to any of several users each having a respective candidate node maintained by the system, the system can compare a timestamp of the new electronic activity with a time zone of the candidate node profiles in order to select or determine one of the node profiles that is associated with the new electronic activity.

In some implementations, the system can use an inferred time zone of a node profile in order to schedule delivery of other information to a user associated with the node profile. For example, notifications, messages, or any other information can be delivered to a user at a time selected based in part on the inferred time zone. This can help to increase a likelihood that the user will receive, view, or act on the delivered information. For example, it may be desirable to provide information such as a notification to the user at a time (or during a window of time) when the user is likely to be able to receive, view, or act on the notification, such as during daytime hours or during typical work hours in the user's time zone. If the notification is instead sent during different hours (e.g., during the night), the user may be less likely to receive, view, or act on the notification. Thus, in some implementations, the system can identify one or more restricted notification periods based on the determined time zone. The restricted notification periods may be any periods that are determined to be periods during which the user is unlikely to receive, view, or act on a notification, such as during the early morning or night time hours in the user's time zone. In some the system can restrict transmission of the notification to the computing device of the user associated with the node profile during the restricted notification period. The system can schedule delivery of the notification to the computing device of the user during a time period that is not restricted, based on the inferred time zone.

According to the embodiments of the systems and methods described herein, the system can parse and process electronic activities to infer or otherwise determine a time zone for a person. The system can assign, correlate, or otherwise associate the time zone with a node profile for the user. The system can use information from various sources of electronic activity for generating temporal distributions of electronic activity for each user. The temporal distributions for each user may each be associated with a respective time interval. The system can identify the time zone for a user based on the temporal distributions. Various other benefits and advantages of the present technical solution described further below.

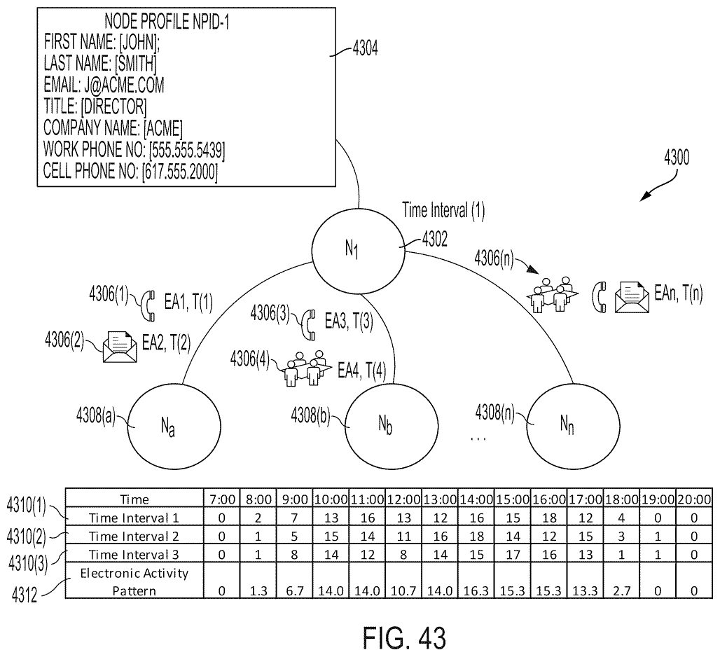

Referring now to FIG. 43, illustrated is a use case diagram 4300 for inferring a time zone of a node profile based on electronic activities. The diagram 4300 depicts a node profile 4304 associated with a user who participates in a plurality of electronic activities 4306 during a time period (e.g., a day, a week, a month, etc.). The user corresponding to node profile 4304 may participate in electronic activities 4306 with various other users corresponding to other nodes 4308a-4308n within the time interval. Each user may include a corresponding node profile 4304 (e.g., a node profile 4304 corresponding to “John Smith”, and node profiles for each of the users who are participants with “John Smith” in electronic activities 4306(1)-4306(n)). In some implementations, at least some of the operations described in connection with the diagram 4300 may be performed by a system such as the node graph generation system 200 of FIG. 4. For example, as described in greater detail above, the system 200 may be configured to generate the node profiles 4304 based on electronic activity. In the use case diagram depicted in FIG. 43, a first user (e.g., “John Smith”) may generate and send a number of electronic activities at various times to various other users. The use case diagram shows a plurality of different types of electronic activities (e.g., phone calls, emails, electronic calendar events, etc.) conducted between John Smith and various other users. As described in greater detail below, the system 200 may be configured to determine, identify, or otherwise infer a time zone for the node profile 4304 based on the electronic activities engaged in by a corresponding user (e.g., John Smith) over the time interval.

The system 200 may be configured to receive, detect, identify, register, collect, or otherwise access electronic activities 4306. Each electronic activity 4306 may be generated, transmitted, or received by at least one participant. In some instances, an electronic activity 4306 may include a plurality of participants. For instance, a user initiating an email to one recipient may be the sole participant of the electronic activity 4306 (e.g., the one participant being the sender of the email). As another example, a conference call, meeting, phone call, etc. may include two or more participants (e.g., attendees or participants of the conference call, meeting, phone call, etc.). In instances in which a phone call is initiated and not answered, the corresponding electronic activity 4306 may have one participant (e.g., the person initiating the phone call). In instances in which a phone call is answered, the corresponding electronic activity 4306 may have two or more participants (e.g., the person initiating the phone call and the person(s) answering the phone call).

The system 200 may be configured to detect, receive, or otherwise identify electronic activities (such as electronic activity 4306) exchanged, transmitted, received, or otherwise engaged in by a user corresponding to a node profile. As described in greater detail above, the system 200 may include an electronic activity ingestor 205 configured to ingest electronic activities 4306 from a plurality of data source providers. The electronic activities 4306 may be received or ingested in real-time or asynchronously as electronic activities are generated, transmitted, or stored by the one or more data source providers. The data source providers each may be or may include a server, which hosts a domain corresponding to one or more of the participants of an electronic activity 4306 (e.g., the sender or the recipient). The system 200 may identify electronic activities 4306 that identify a sender, one or more recipient(s), and include a body or content. As described in greater detail below, the electronic activity parser 210 of the system 200 may be configured to parse the identified electronic activities. The electronic activity parser 210 may parse the electronic activity to identify a timestamp for each electronic activity 4306 sent, generated, or otherwise initiated by a user associated with a node profile 4304. The system 200 may be configured to generate a temporal distribution for each time interval in a time period based on the timestamps. The system 200 may be configured to determine an electronic activity pattern based on the temporal distributions over the time period. The system 200 may be configured to identify a region of the temporal distribution corresponding to a schedule of the user by applying a region identification policy to the electronic activity pattern.

The system 200 may be configured to select, determine, locate, or otherwise identify each electronic activity 4306 sent, received, or otherwise engaged in by the user corresponding to the node 4302. The system 200 may be configured to identify each electronic activity 4306 associated with the node profile 4304 for the user. The node profile 4304 may include a plurality of fields having values corresponding to accounts of the user. For instance, in the example depicted in FIG. 43, the node profile 4304 includes a value “J@ACME.COM” for a field associated with an email address, a value “555.555.5439” for a field associated with a work phone number, and a value “617.555.2000” for a field associated with a cell phone number. The electronic activity parser 210 may parse each electronic activity 4306 to identify a set of electronic activities in which John Smith was a participant based on which electronic activities indicate a sender or recipient having a value that matches at least one of the values of a corresponding field associated with the node profile 4304 (e.g., a phone call dialed by or answered via a phone number having a value that matches the value corresponding to the field associated with work phone number or cell phone number, an email sent via an email address having a value that matches the value corresponding to the field associated with the email address, and so forth).

The system 200 may parse each of the electronic activities 4306 sent, received, or otherwise engaged in by the user corresponding to the node 4302. In some embodiments, the electronic activity parser 210 may parse a header or metadata for an electronic activity 4306 to determine information relating to the electronic activity 4306. For example, the electronic activity parser 210 may parse the header or metadata for each electronic activity 4306 to identify a timestamp associated with each electronic activity 4306. As stated above, each electronic activity 4306 may include a corresponding timestamp. The timestamp may be or may indicate the time at which the electronic activity 4306 was generated, sent, received, stored, etc. The electronic activity parser 210 may be configured to parse each electronic activity 4306 to identify the timestamp associated with each respective electronic activity.

In some embodiments, the system 200 may be configured to determine a type of electronic activity for each electronic activity 4306 sent, received, or otherwise engaged in by the user corresponding to the node 4302. In some embodiments, the tagging engine 265 may be configured to tag each electronic activity 4306 with a tag corresponding to the type of electronic activity. For instance, the tagging engine 265 may tag an electronic activity 4306 as an email if the electronic activity 4306 was transmitted via an electronic account included as a value of an email address field of the node profile 4304. The system 200 may determine the type of electronic activity for each electronic activity 4306 sent, received, or otherwise engaged in by the user corresponding to the node 4302 based on the tag for each electronic activity. As another example, the electronic activity parser 210 may be configured to determine the type of electronic activity for each electronic activity 4306. As described above, the electronic activity parser 210 may parse electronic activity to identify a set of electronic activities in which a user was engaged based on which electronic activities indicate a sender or recipient having a value that matches at least one of the values of a corresponding field associated with the node profile 4304. In some embodiments, the electronic activity parser 210 may store an association between each field of the node profile 4304 and a corresponding type of electronic activity. The electronic activity parser 210 may identify the type of electronic activity for each electronic activity in the set based on the association between the field of the node profile 4304 and which value was used for including the electronic activity 4306 in the set. For example, the electronic activity parser 210 may store an association between the field “EMAIL” and the type of electronic activity “EMAIL.” The electronic activity parser 210 may identify electronic activities for including in the set of electronic activities linked to the node profile 4304 based on which electronic activities were sent or received by “J@ACME.COM.” The electronic activity parser 210 may similarly identify the type of each electronic activity in the set as an email based on which electronic activities which were sent or received by “J@ACME.COM” and the association with the corresponding field “EMAIL” and type of electronic activity “EMAIL.” In a similar manner, the electronic activity parser 210 may also determine whether each electronic activity 4306 is a phone call, a text message, an electronic calendar event, etc. In some implementations, the electronic activity parser 210 can determine a type for an electronic activity 4306 based on a data format or file extension of a computer file associated with the electronic activity 4306.

In some embodiments, the electronic activity parser 210 may be configured to parse the electronic activity 4306 to determine a location corresponding to the user when the user engaged in the electronic activity. The electronic activity 4306 may include metadata corresponding to an IP address of the client device which was used for generating, transmitting, sending, or otherwise engaging in the electronic activity 4306. As another example, the electronic activity 4306 may include metadata corresponding to a network address, network name, etc. to which the client device was connected when the user was engaging in the electronic activity. The electronic activity parser 4306 may parse the metadata to extract such information for identifying, determining, or otherwise inferring a location of the user when the user engaged in the electronic activity. The electronic activity parser 210 may maintain, include, or otherwise access a list, ledger, database, etc. of IP addresses of enterprise client devices, enterprise network addresses, enterprise network names, etc. associated with the node profile 4304 for the user. The electronic activity parser 210 may compare the data extracted from an electronic activity 4306 to the data included in such a database to determine whether the electronic activity was engaged in on the enterprise client device of the user, the enterprise network, etc. If the electronic activity parser determines that the IP address, network address, network name, etc. matches the data in the database, the electronic activity parser 210 may determine the electronic activity was engaged in (e.g., generated, sent, received, etc.) at a location associated with the enterprise. Where the electronic activity parser 210 does not identify a match, the electronic activity parser 210 may determine the electronic activity was engaged in at a location other than the location of the enterprise. The electronic activity parser 210 may determine, identify, or infer the location in which the user engaged in the electronic activity for applying weights to the electronic activity in generation of an electronic activity pattern for the user.

The system 200 may be configured to arrange, sort, filter, compile, or otherwise identify the electronic activities 4306 generated by the user within a time period. In some embodiments, the system 200 may include a temporal distribution generation engine. The temporal distribution generation engine may be or include any script, file, program, application, set of instructions, or computer-executable code that is configured to determine, identify, compile, produce, or otherwise generate a temporal distribution of electronic activity for a user within the time period. The time period may be, for instance, a day (e.g., a 24 hour period), a week (e.g., a working week from Monday through Friday), a month (e.g., a 45-day period), and so forth. In some implementations, the temporal distribution generation engine may be embodied on or otherwise be a component or element of the electronic activity parser 210. The temporal distribution generation engine may be configured to generate the temporal distribution of electronic activity for a series of time intervals within a time period (e.g., within a month window, a 90 day window, a 180 day window, and so forth). Hence, a time period may be made up of a group of time intervals.

The temporal distribution generation engine may be configured to generate the temporal distribution of electronic activity based on the identified timestamps for each of the electronic activity in the set of electronic activity for a user. The electronic activity parser 210 may be configured to identify the timestamp for each electronic activity generated, sent, received, or otherwise engaged in by the user. The temporal distribution generation engine may be configured to generate the temporal distribution by compiling each electronic activity engaged in by the user having a timestamp which falls within a time interval. The temporal distribution generation engine may sort, compile, or otherwise filter each of the electronic activities within the set of electronic activities by timestamp. The temporal distribution generation engine may maintain, for each time interval within the time period, a subset of electronic activities having timestamps which fall within the respective time intervals. Hence, a given time interval may have any number of electronic activities based on which electronic activities were engaged in by the user within that time interval.

Continuing the example shown in FIG. 43, John Smith is shown to have participated in two electronic activities (a phone call EA1 and an email EA2) with a second node (e.g., N2 at T(1) and T(2), respectively) and two electronic activities (a phone call EA3 and a meeting EA4) with a third node (e.g., N3 at T(3) and T(4), respectively). John Smith may engage in any number of electronic activities at various times within a first time interval (e.g., Time Interval (1)). The temporal distribution generation engine may compile each of the electronic activities engaged in by John Smith for the first time interval based on the timestamps (e.g., T(1)-T(N)) which fall within the time interval (e.g., Time Interval (1)). The temporal distribution generation engine may sort, arrange, or organize each of the electronic activities compiled for the first time interval by their respective timestamps.

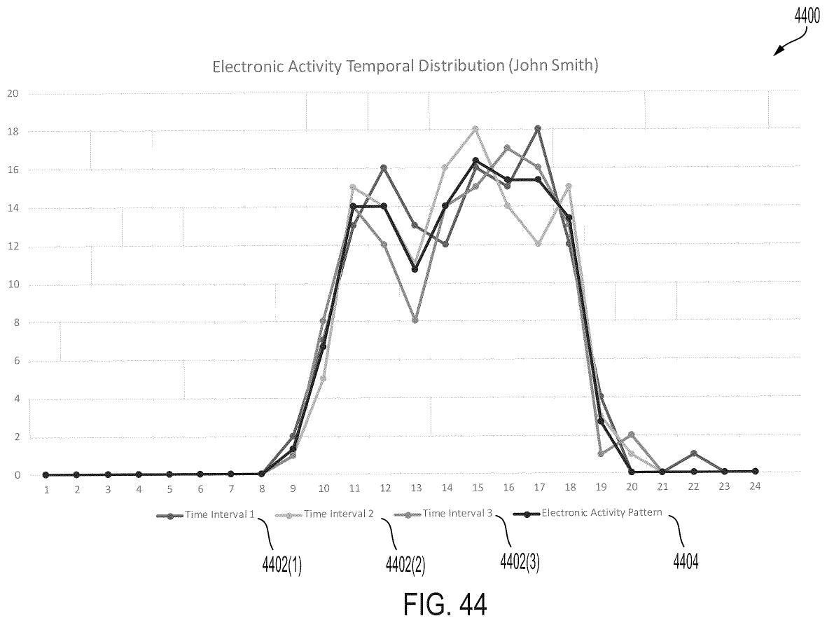

Referring now to FIGS. 43 and 44, the temporal distribution generation engine may compile, produce, or otherwise generate data corresponding to a graphical representation 4402 of the temporal distributions. Specifically, FIG. 44 depicts a graph 4400 showing a plurality of graphical representations 4402 of temporal distributions 4310 of electronic activity for various time periods. The temporal distribution generation engine may generate a temporal distribution 4310(1) of electronic activities for the first time period (Time Period (1)). In some implementations, each electronic activity within the subset of electronic activities falling within the first time period may be arranged within a time interval (e.g., an hour) of the first time period (e.g., a 24 hour period). The temporal distribution generation engine may generate the temporal distribution 4310(1) by grouping each electronic activity in the subset by which time interval of the first time period the respective timestamps fall within. As shown in FIGS. 43 and 44, for the first time period (Time Period (1)), John Smith may not have engaged in any electronic activity (e.g., did not send, initiate, or otherwise actively participate in any electronic activity) between time intervals beginning at 00:00-07:00. However, in the example shown in FIGS. 43 and 44, John Smith engaged in two electronic activities (for instance, participated in a call, responded to an email, etc.) within the time interval between 8:00-8:59, seven electronic activities within the time interval between 9:00-9:59, 13 electronic activities within the time interval between 10:00-10:59, and so forth through 18:00-18:59 (e.g., 6:00P-6:59 PM). Following 18:59, John Smith may not have engaged in any electronic activity for the rest of the time period (e.g., from 19:00-23:59). The graphical representation 4402(1) corresponding to the temporal distribution 4310(1) shows the electronic activities generated, initiated, participated in, or otherwise engaged in by the user (e.g., John Smith) over the first time period. As shown in FIG. 44, the temporal distribution generation engine may generate graphical representations 4402(1)-2902(3) of the temporal distributions 4310(1)-2810(3) of electronic activity at various time periods (Time Period (1)-Time Period (3)).

The temporal distribution generation engine may be configured to generate, for each time period, a respective temporal distribution 4310. While charts representing three temporal distributions are depicted in FIG. 43 and FIG. 44, it is noted that the temporal distribution generation engine may generate any number of temporal distributions 4310 depending on the number of time intervals within the time period. In some implementations, the temporal distribution generation engine may generate temporal distributions 4310 for time periods corresponding to business days. The temporal distribution generation engine may maintain, include, or otherwise access a list of business days corresponding to an enterprise associated with the user. The list of business days may be, for instance, Monday through Friday. In some implementations, the list of business days may be a preset list of business days. In some embodiments, a person associated with the enterprise, such as an administrator, the user, etc., may update, modify, or otherwise change the preset list of business days based on scheduled non-business days (e.g., holidays), days off, and so forth. In some embodiments, the temporal distribution generation engine may be configured to automatically detect (e.g., based on deviations from an electronic activity pattern for the user) non-business days. The temporal distribution generation engine may generate temporal distributions for each time period which corresponds to a business day. The temporal distribution generation engine may forego generation of (or disregard, delete, or otherwise remove already generated) temporal distributions 4310 of electronic activity corresponding to non-business days.

In some embodiments, the system 200 may compare at least one temporal distribution for a node profile to one or more other temporal distributions stored in a database or data structure and associated with a respective time zone. The database may include electronic activity temporal distributions corresponding to each of a plurality of time zones (e.g., 24 temporal distributions each corresponding to a respective time zone). In some implementations, the stored temporal distributions may be referred to as temporal distribution templates. The system 200 may identify the temporal distribution template included in the database that most closely matches at least one temporal distribution of the user. For example, the system 200 may identify which temporal distribution template of the database most closely matches the temporal distribution of the user based on which temporal distribution template tracks the temporal distribution of the user, which temporal distribution template has or includes upticks and downticks in electronic activity within the same time frame as the temporal distribution of the user, and so forth. When a match is determined, the system 200 may infer that the time zone for the user is the same as the time zone of the matching temporal distribution template, based on the match.

In some implementations, the temporal distribution templates can be generated or selected based on electronic activities associated with node profiles having known time zone information. For example, to generate a temporal distribution template for a given time zone, the system 200 may identify at least one node profile that is known to be associated with a user in that time zone. The system 200 may then access a set of electronic activities for the identified node profile to determine at least one temporal distribution of electronic activities for the node profile, in a manner similar to that described above, and the at least one temporal distribution may be stored as a temporal distribution template for that time zone. In some implementations, the system 200 may store more than one temporal distribution template for a given time zone. For example, the system 200 may store a respective temporal distribution template for each of a plurality of different industries, job titles, or any other characteristics of node profiles. In some implementations, typical electronic activity patterns may vary across industries or job titles, even within the same time zone. Thus, in order to infer a time zone of a user based on the user's electronic activities, it may be useful to compare the user's temporal distribution of electronic activities to a corresponding temporal distribution template representing one or more node profiles of users in the same industry or with the same job title. To achieve this, the system 200 may generate and store temporal distribution templates for different job titles, industries, or any other criteria within a given time zone. To infer a time zone for a particular node profile, the system 200 may select one or more temporal distribution templates to compare with the temporal distribution of the node profile based on match between an industry or job title of the node profile and a corresponding industry or job title of the one or more temporal distribution templates.

In some implementations, instead of comparing a single temporal distribution of electronic activities of a node profile with another temporal distribution (e.g., a temporal distribution template), the system 200 may determine a temporal distribution pattern for the node profile based on a plurality of temporal distributions, and the pattern can be compared to a corresponding pattern known to be associated with particular time zones in order to determine a match. For example, the system 200 may be configured to generate, identify, or otherwise determine an electronic activity pattern for the user based on more than temporal distribution of electronic activities for the user. In some embodiments, the system 200 may include a pattern determination engine. The pattern determination engine may be or include any script, file, program, application, set of instructions, or computer-executable code that is configured to generate, identify, compile, produce, or otherwise determine an electronic activity pattern for the user within the time period. The pattern determination engine may determine the electronic activity pattern based on each of the temporal distributions 4310 generated by the temporal distribution generation engine that fall within the time period. The pattern determination engine may determine the electronic activity pattern by applying a pattern determination policy to each of the temporal distributions within the time period. The pattern determination policy may be a rule-based policy, a probabilistic model, etc. The pattern determination policy may be generated by an administrator defining the rules, generated via a machine learning algorithm using a training set of temporal distributions and corresponding electronic activity patterns, and so forth.

The pattern determination engine may be configured to apply the pattern determination policy to each of the temporal distributions within the time period for generating the electronic activity pattern for the user. As described in greater detail below, the system 200 may be configured to determine a region of the time interval corresponding to a schedule of the user based on the electronic activity pattern for the user.

The pattern determination engine may use the pattern determination policy to compute an average, mean, or other statistic of electronic activity for each time frame within a respective time interval in the time period. In some embodiments, the pattern determination engine may use the pattern determination policy to filter one or more data outliers in the temporal distribution (e.g., a number of electronic activities exceeding a standard deviation from the average or mean of electronic activity within a respective time frame). In some embodiments, the pattern determination engine may use the pattern determination policy to filter electronic activities within a respective time frame having a mean or average less than one electronic activity. Continuing the example depicted in FIG. 43, the pattern determination engine may apply the pattern determination policy to each of the temporal distributions 4310(1)-2810(3) to identify an electronic activity pattern 4312. The electronic activity pattern 4312 may be an average of electronic activity within each time frame (e.g., 8:00-19:59) for each time interval (Time Interval (1)-Time Interval (3)). The pattern determination engine may filter, from the electronic activity pattern 4312, data points for time frames with average electronic activity less than one (e.g., time frame 19:00-19:59 has an average electronic activity of 0.66, which is less than one). The pattern determination engine may be configured to generate a graphical representation 4404 of the electronic activity pattern 4312 on the graph 4400. As can be seen in FIG. 44, the electronic activity pattern 4312 may track, trend, or otherwise follow the temporal distributions of electronic activity for each time interval within the time period.

In some implementations, the pattern determination engine may use the pattern determination policy to leverage data from the electronic activity for applying weights to different electronic activities within the respective temporal distributions. In some embodiments, the pattern determination engine may use the pattern determination policy to apply weights to electronic activity based on the determined type of electronic activity, the complexity of electronic activity, and/or the location in which the electronic activity was engaged.

The pattern determination engine may use the pattern determination policy to apply weights to electronic activity within the temporal distributions based on the determined type of electronic activity. The pattern determination engine may use the pattern determination policy to use the identified type of electronic activity within the time interval for assigning weights to each electronic activity. In such embodiments, the weights may be indicative of, correspond to, or otherwise associated with a level of engagement of the user in engaging in the electronic activity. For instance, the pattern determination engine may use the pattern determination policy to apply weights that favor electronic activity corresponding to participating in meetings greater than electronic activity corresponding to emails. As another example, the pattern determination engine may use the pattern determination policy to apply weights that favor electronic activity corresponding to phone calls or conference calls greater than electronic activity corresponding to emails. The pattern determination engine may use the pattern determination policy to generate the electronic activity pattern as described above following applying weights to the respective electronic activities in each temporal distribution based on the type of electronic activity.

The pattern determination engine may use the pattern determination policy to apply weights to the electronic activity based on the complexity of each electronic activity. The pattern determination engine may use the pattern determination policy to use the identified complexity of electronic activity within the time interval for assigning weights to each electronic activity. In such embodiments, the weights may be indicative of, correspond to, or otherwise be associated with a level of engagement of the user in engaging in the electronic activity. The pattern determination engine may use the pattern determination policy to leverage a language complexity score and/or word/character count generated, determined, or otherwise identified by the electronic activity parser 210 for applying weights to the electronic activity. The pattern determination engine may use the pattern determination policy to include, maintain, or otherwise access a database or set of rules corresponding to language complexity scores, word or character counts, etc. and associated weights. For instance, the pattern determination engine may use the pattern determination policy to apply weights which favor electronic activity having a higher complexity score, higher word or character count, etc. Hence, the weights may increase with the language complexity score, word count, character count, etc. As the language complexity score, word count, character count, etc. increases, the level of engagement of the user in engaging in the electronic activity may be presumed, assumed, or inferred to correspondingly increase. The pattern determination engine may use the pattern determination policy to generate the electronic activity pattern as described above following applying weights to the respective electronic activities in each temporal distribution based on the complexity of electronic activity.

The pattern determination engine may use the pattern determination policy to apply weights to the electronic activity based on the location in which the electronic activity was generated. The pattern determination engine may use the pattern determination policy to use the location in which the electronic activity was generated, sent, or otherwise engaged in by the user for assigning weights to each electronic activity. For instance, the pattern determination engine may use the pattern determination policy to apply weights which favor electronic activity engaged in at a location associated with an enterprise corresponding to the user. As another example, the pattern determination engine may use the pattern determination policy to apply weights which disregard, discard, filter, or otherwise ignore electronic activity generated at a location other than a location associated with the enterprise. The pattern determination engine may use the pattern determination policy to generate the electronic activity pattern as described above following applying weights to the respective electronic activities in each temporal distribution based on the location in which the user was engaged in the electronic activity.

The system 200 may be configured to determine, select, or otherwise identify a region of interest of the electronic activity pattern. In some embodiments, the system 200 may include a region identification engine to perform this functionality. The region identification engine may be or include any script, file, program, application, set of instructions, or computer-executable code that is configured to generate, determine, compile, select, or otherwise identify a particular region of the electronic activity pattern for the user. The region may be associated with or otherwise correspond to an inferred schedule of the user. The region identification engine may apply a region identification policy to the electronic activity pattern for identifying the region. The region identification engine may apply the region identification policy to the electronic activity pattern for determining which portions of the time interval the user is usually, normally, typically, etc. engaged in electronic activity. The system 200 may identify the region for inferring a work schedule of the user with the work schedule being correlated with the time interval in which the user is typically engaged in electronic activity.

In some embodiments, the region identification policy may be or include a probabilistic model. The probabilistic model may be generated via a machine learning algorithm trained based on a plurality of electronic activity patterns and corresponding work schedules. The machine learning algorithm may receive, as an input, a plurality of electronic activity patterns for different users and their respective work schedules (which may be manually entered, for instance.). The machine learning algorithm may output a probabilistic model which is configured to receive a given electronic activity pattern for a user and output a corresponding region of a time interval which is likely to correspond to a work schedule for the user. The region identification policy may refine the probabilistic model over time as subsequent work schedules are verified. The region identification policy may update the probabilistic model by retraining the probabilistic model via the machine learning algorithm.

In some embodiments, the region identification policy may leverage thresholds of electronic activity within a time frame of the time interval for identifying the region. The region identification policy may include, maintain, or otherwise access a first threshold corresponding to a first time of the time interval and a second threshold corresponding to a second time of the time interval. The region identification policy may be used by the system to apply the first and second thresholds to the electronic activity pattern to identify the first and second time of the time interval. In some embodiments, the threshold may be or correspond to a change in electronic activity over a time frame. In some embodiments, the thresholds may correspond to an average amount of electronic activity over two adjacent time frames. In some embodiments, the thresholds may correspond to a slope of the graphical representation 4404 of the electronic activity pattern. The region identification policy may be used by the system to apply the first and second thresholds to the electronic activity pattern to identify a first time corresponding to an uptick in electronic activity and a second time corresponding to a downtick in electronic activity engaged in by the user. The region identification policy may be used by the system to select the region of the time interval as spanning the first and second time.

In some embodiments, the region identification policy may be used by the system to compare the electronic activity pattern for a node profile to one or more other electronic activity patterns stored in a database or data structure and associated with respective regions of the time interval. The database may include electronic activity patterns and respective regions of the time interval similar to the datasets used to train the machine learning algorithm for producing the probabilistic model as described above. The region identification policy may be used by the system to compare the electronic activity pattern of the user with the other electronic activity pattern(s) included in the database. The region identification policy may identify the region of the time interval based on which electronic activity pattern included in the database most closely matches the electronic activity pattern of the user. The region identification policy may be used by the system to identify which electronic activity pattern of the database most closely matches the electronic activity pattern of the user based on which electronic activity pattern tracks the electronic activity pattern of the user, which electronic activity pattern has or includes upticks and downticks in electronic activity within the same time frame as the electronic activity pattern of the user, and so forth. The region identification policy may be used by the system to identify the region of the time interval corresponding to the electronic activity pattern which most closely matches the electronic activity pattern of the user.

In some embodiments, the system 200 may compare at least one electronic activity pattern for a node profile to one or more other electronic activity pattern stored in a database or data structure and associated with a respective time zone. The database may include electronic activity patterns corresponding to each of a plurality of time zones (e.g., 24 electronic activity patterns each corresponding to a respective time zone). In some implementations, the stored electronic activity patterns may be referred to as electronic activity pattern templates. The system 200 may identify the electronic activity pattern template included in the database that most closely matches at least one electronic activity pattern of the user. For example, the system 200 may identify which electronic activity pattern template of the database most closely matches the electronic activity pattern of the user based on which electronic activity pattern template tracks the electronic activity pattern of the user, which electronic activity pattern template has or includes upticks and downticks in electronic activity within the same time frame as the electronic activity pattern of the user, and so forth. When a match is determined, the system 200 may infer that the time zone for the user is the same as the time zone of the matching electronic activity pattern template, based on the match.

In some implementations, the electronic activity pattern templates can be generated or selected based on electronic activities associated with node profiles having known time zone information. For example, to generate an electronic activity pattern template for a given time zone, the system 200 may identify at least one node profile that is known to be associated with a user in that time zone. The system 200 may then access a set of electronic activities for the identified node profile to determine a plurality of temporal distributions of electronic activities for the node profile, and may further determine an electronic activity pattern for the node profile. The system 200 may store the determined electronic activity pattern as an electronic activity pattern template for that time zone. In some implementations, the system 200 may store more than one electronic activity pattern template for a given time zone. For example, the system 200 may store a respective electronic activity pattern template for each of a plurality of different industries, job titles, or any other characteristics of node profiles. In some implementations, typical electronic activity patterns may vary across industries or job titles, even within the same time zone. Thus, in order to infer a time zone of a user based on the user's electronic activities, it may be useful to compare the user's electronic activity pattern to a corresponding electronic activity pattern template representing one or more node profiles of users in the same industry or with the same job title. To achieve this, the system 200 may generate and store electronic activity pattern templates for different job titles, industries, or any other criteria within a given time zone. To infer a time zone for a particular node profile, the system 200 may select one or more electronic activity pattern templates to compare with the electronic activity pattern of the node profile based on match between an industry or job title of the node profile and a corresponding industry or job title of the one or more electronic activity pattern templates.

In some embodiments, the system 200 may be configured to identify values from field-value pairs of the node profile 4304 associated with the user. The system 200 may be configured to identify a value corresponding to a field-value pair associated with a job title for the user, a field-value pair associated with an industry of the enterprise or company in which the user is employed, a location of the enterprise or company, etc. The region identification engine may identify the region associated with the schedule of the user based on the values from the node profile 4304 for the user. In some implementations, the region identification engine may identify the region based on the job title of the user as reflected in the node profile 4304 of the user. For instance, where the job title indicates a higher level position, the user may typically work longer or more consistent hours. On the other hand, where the job title indicates a lower level position, the user may work fewer hours, may be part-time, may work less consistent hours, and so forth. As another example, the industry in which the user works may reflect more work from home options, more flexible working hours, time-shifted hours (e.g., relative to other industries in the same time zone), and so forth. The region identification engine may leverage the data from the node profile 4304 of the user for modifying, adapting, or otherwise changing the region of the time interval. The region identification engine may expand the region when the user is a higher level position, adjust one or more weights of the temporal distribution (e.g., the weights based on the location of the user) based on the industry, and so forth. Each of these implementations may use data from the node profile 4304 of the user to more accurately select or modify the region of the time interval corresponding to the schedule of the user.

The region identification engine may be configured to store an association between the node profile 4304 of the user and the identified region of the time interval corresponding to the schedule of the user. The region identification engine may store the association in a database, memory, server, or other data structure. In some embodiments, the identified region may be static (e.g., once the region is identified, the region remains the same). In some embodiments, the identified region may be dynamic (e.g., the region may be changed over time). In some embodiments, the region identification engine may update the region at various points in time. The region identification engine may update the region on a schedule (e.g., once every six months, once every year, etc.). The region identification engine may update the region upon occurrence of a condition which triggers the update. The condition may be a job change, a promotion, and so forth. The system 200 may identify such conditions based on changes in confidence score of values corresponding to fields of the node profile 4304, as described above in Section 1(D) above.

In some embodiments, the system 200 may leverage the region associated with the node profile 4304 of the user for identifying an event condition. In some embodiments, the system 200 may include an event condition detection engine. The event condition detection engine may be or include any script, file, program, application, set of instructions, or computer-executable code that is configured to determine, identify, or otherwise detect an event condition based on a comparison of temporal distributions to an electronic activity pattern of a user. The event condition may be or correspond to vacation, a sick day, leave (e.g., maternity, paternity, adoption, etc.), and so forth. Following generation of the electronic activity pattern by the pattern determination engine, the event condition detection engine may leverage the electronic activity pattern to detect event conditions. In some embodiments, the event condition detection engine may include, maintain, or otherwise access an event condition detection policy. The event condition detection policy may be or include a rule-based policy, a probabilistic model, etc. The event condition detection engine may be configured to apply the event condition detection policy to subsequent temporal distributions to determine whether the temporal distribution satisfies the policy for detecting a corresponding event condition.

In some embodiments, the event condition detection policy may include a machine learning model. The machine learning model may be a model which is generated, produced, or otherwise trained to classify temporal distributions of electronic activity. The machine learning model may be trained based on data sets, samples, or other training examples of electronic activity patterns or temporal distributions (or series of temporal distributions) and corresponding labels associated with respective event conditions. For instance, the machine learning model may be trained with a set of training examples corresponding to a first event condition (e.g., a sick day) using a plurality sample temporal distributions corresponding to the first event condition (e.g., the sick day). Each sample temporal distribution may be tagged or labeled with the respective event condition (e.g., a sick day label). Continuing this example, where a person is taking a sick day, the overall number of electronic activity engaged in by the person may decrease for the sick day. The machine learning model may be trained with temporal distributions corresponding to the sick day such that, where a temporal distribution corresponding to a sick day is provided as an input to the machine learning model, the machine learning model is configured to generate a label (or a confidence score corresponding to the label) identifying the temporal distribution as a sick day. As another example, the machine learning model may be trained with a set of training examples corresponding to a second event condition (e.g., a vacation) using a plurality sample temporal distributions in series corresponding to the second event condition (e.g., the vacation). Each sample temporal distribution may be tagged or labeled with the respective event condition (e.g., a vacation label). Continuing this example, where a person is taking a vacation, the overall number of electronic activity engaged in by the person may decrease to below a threshold for a series of days in a row (e.g., a week, for instance). The machine learning model may be trained with the series of temporal distributions corresponding to the vacation such that, where a series of temporal distributions of a user corresponding to a vacation is provided as an input to the machine learning model, the machine learning model is configured to generate a label (or a confidence score corresponding to the label) identifying each of the temporal distributions as vacation time. As described in greater detail above, the temporal distribution generation engine may be configured to generate various temporal distributions of electronic activity for various time intervals.

In some embodiments, the temporal distribution generation engine may generate a temporal distribution for a set of electronic activities engaged in by the user after generation of the electronic activity pattern (e.g., at a subsequent point of time). The temporal distribution generation engine may generate the temporal distribution for each electronic activity engaged in by the user during a subsequent time interval (referred to herein as a subsequent temporal distribution). The event condition detection engine may be configured to apply the event condition detection policy to the subsequent temporal distribution to determine whether the subsequent temporal distribution satisfies the event condition detection policy for identifying an event condition.

In some embodiments, the event condition detection engine may be configured to identify, determine, or otherwise detect the event condition corresponding to a temporal distribution based on one or more characteristics of the subsequent temporal distribution satisfying a corresponding characteristic associated with the respective event condition. The event condition detection engine may maintain, include, or otherwise access a plurality of characteristics associated respective labels for corresponding event conditions. For instance, a characteristic associated with an event condition for a sick day may indicate a drop in electronic activity engaged in by the user for the time interval below a standard deviation (or other threshold) from an average electronic activity engaged in by the user. The event condition detection engine may access a characteristic associated with the event condition (e.g., sick day). The event condition detection engine may determine whether the subsequent temporal distribution of the user satisfies the characteristic (e.g., the average electronic activities engaged in by the user in the subsequent temporal distribution is outside of a standard deviation from the average electronic activity engaged in by the user). The event condition detection engine may detect the event condition (e.g., the sick day) based on the temporal distribution satisfying the characteristic. As another example, where the user takes a vacation day, a temporal distribution for a business day may include less than threshold number of electronic activities engaged in by the user (e.g., zero or close to zero electronic activities). The event condition detection engine may store, include, maintain, or otherwise access a characteristic associated with the condition corresponding to a vacation day (e.g., average electronic activities or number of electronic activities engaged in by a given user falling below a threshold). The event condition detection engine may be configured to detect an event condition corresponding to a vacation day based on the subsequent temporal distribution satisfying the corresponding characteristic associated with the vacation day event condition.

The event condition detection engine may be configured to store an association between the node profile 4304 of the user and the event condition. The event condition detection engine may be configured to store the association between the node profile 4304 and event condition to more accurately reflect the user's schedule. The event condition detection engine may be configured to store the association between the node profile 4304 and event condition and in association with the region of the time interval corresponding to the user's work schedule. In some embodiments, the system 200 may be configured to generate a report corresponding to a schedule of the user. The report may be a schedule of the user across the time period. For instance, the system 200 may be configured to generate the report to reflect each time interval in which the user worked, and reflect the event conditions. The system 200 may be configured to generate a weekly report, a monthly report, etc. The system 200 may be configured to communicate the report to the user, to a manager, leader, or other employee managing the user, and so forth.

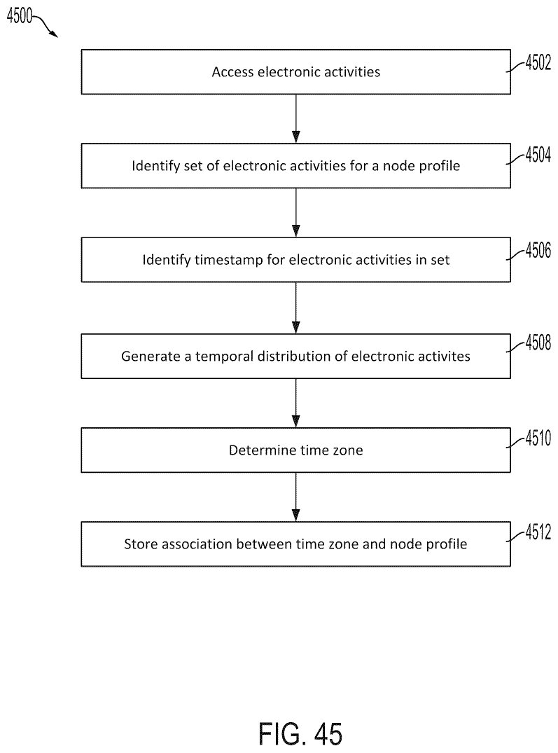

Referring now to FIG. 45, illustrated is a flow diagram of a method 4500 of inferring a time zone of a node profile using electronic activities. The method 4500 can be implemented or performed using any of the components described above in conjunction with FIGS. 1-29 (e.g., the node graph generation system 200) or the server system 4800 detailed below in conjunction with FIG. 48. The method 4500 may include accessing electronic activities (4502). The method 4500 may include identifying a set of electronic activities for a node profile (4504). The method 4500 may include identifying a timestamp for electronic activities in the set (4506). The method 4500 may include generating a temporal distribution of electronic activities (4508). The method 4500 may include determining a time zone (4510). The method 4500 may include storing an association between the time zone and the node profile (4512).

The method 4500 may include accessing electronic activities (4502). In some embodiments, the system 200 may access a plurality of electronic activities transmitted or received by a plurality of participants corresponding to one or more respective electronic accounts associated with a plurality of data source providers. The system 200 may access the electronic activities as they are generated, sent, received, exchanged, or otherwise engaged in by the participants. In some embodiments, the system 200 may access the electronic activities when they are ingested into the system 200 from the data source provider(s). Each of the electronic activities may include metadata corresponding to, identifying, or otherwise used for identifying participants of the electronic activity. For instance, the metadata may include a sender/initiator, recipient, other participants, etc. of the electronic activity. Each of the electronic activities may include a timestamp. The timestamp may indicate the time in which the electronic activity was generated, sent, received, or otherwise engaged in by the participant(s).

The method 4500 may include identifying a set of electronic activities for a node profile (4504). In some embodiments, the system 200 may identify a set of electronic activities for a node profile engaged in by a participant of the plurality of participants linked to the node profile within a time period. Each node profile may include, for instance, a value for a field-value pair corresponding to an email address, a value for a field-value pair corresponding to a phone number, etc. The system 200 may identify the set of electronic activities for the node profile based on which electronic activities include a matching value from the metadata in the node profile. As described above, the metadata may include a sender, recipient(s), etc. The system 200 may cross-reference the values from the metadata with data from the node profiles to identify the set of electronic activities for the node profile. The system 200 may identify the set of electronic activities within a time period. The time period may be, for instance, a 45-day window, a 90-day window, a 180-day window, and so forth.

In some embodiments, the system 200 may determine a type of each electronic activity engaged in by a participant linked to the node profile within a time period. The system 200 may select the electronic activities for the set having a specific type or types, and may ignore or discard electronic activities having other types. For example, the system 200 may select activities having a type corresponding to electronic calendar events, but may discard electronic activities corresponding to text messages. The system 200 may determine the type of each electronic activity engaged in by determining which value for the field-value pair of the node profile was used for associating the electronic activity to the node profile. The system may correlate specific fields of a node profile with specific types of electronic activity (e.g., field corresponding to email address with an email electronic activity type, field corresponding to work phone number with a phone call electronic activity type, and so forth). The system 200 may identify the electronic activity type for each electronic activity engaged in by the user within the time period. The system 200 may include, in the set of electronic activity, a subset of type(s) of electronic activity. For instance, in some embodiments, the system 200 may limit the set to include only emails and phone calls, as such electronic activity may be more typical of a schedule for a user. In some implementations, the system 200 may limit the set to include electronic calendar events, as such events may be more indicative of a user's time zone than other events. For example, a user may be more likely to participate in other types of electronic activities, such as emails, outside of the user's normal work hours, and therefore such electronic activities may be less useful for determining the user's time zone. On the other hand, a user may be less likely to attend appointments outside of the user's normal work hours (e.g., during the middle of the night), and therefore such electronic activities may be more useful for inferring the user's time zone.

The method 4500 may include identifying a timestamp for electronic activities in the set (4506). In some embodiments, the system 200 may identify a timestamp at which each electronic activity was sent or received in the set of electronic activities (e.g., identified at 4504). The system 200 may parse the metadata of each electronic activity in the set to identify the timestamp for each electronic activity in the set. The timestamp may be a time in which the electronic activity was sent, received, or otherwise engaged in by the user. The system 200 may sort each of the electronic activities by their respective timestamps. Hence, each electronic activity may be sorted or otherwise arranged in chronological order.

The method 4500 may include generating a temporal distribution of electronic activities (4508). In some embodiments, the system 200 may generate a temporal distribution of electronic activity for each time interval within the time period based on respective timestamps of each electronic activity of the set of electronic activities within the time interval. The system 200 may separate each electronic activity within the set into subsets of the electronic activities. Each time interval may correspond to, for instance, one day of the time period, one week of the time period, etc. The system 200 may maintain, for each time period, a set of time intervals. The system 200 may generate the temporal distribution responsive to identifying the electronic activities in the set (e.g., at 4506). The system 200 may generate the temporal distribution for each time interval of the time period. The system 200 may generate the temporal distribution in real-time. The system 200 may generate the temporal distribution at the end of each time interval. The system may generate the temporal distribution of electronic activities by compiling, for each time frame (e.g., 15 minute, half hour, hour, etc.) within the time interval, the number of electronic activities engaged in during the respective time frame. Hence, the temporal distribution may reflect the number of electronic activities engaged in by the user across the time interval.

In some implementations, the method 4500 may include determining an electronic activity pattern. In some embodiments, the system 200 may determine an electronic activity pattern based on the temporal distribution of electronic activity for each time interval within the time period. The system 200 may determine the electronic activity pattern following generation of each temporal distribution (e.g., at each iteration of 4508). The system 200 may determine the electronic activity pattern following generation of a temporal distribution for each time interval within the time period. The system 200 may generate the electronic activity pattern based on each of the temporal distributions (e.g., generated at 4508) that fall within the time period. The system 200 may determine the electronic activity pattern by applying a pattern determination policy to each of the temporal distributions within the time period. The pattern determination policy may be a rule-based policy, a probabilistic model, etc. The pattern determination engine may be configured to apply the pattern determination policy to each of the temporal distributions within the time period for generating the electronic activity pattern for the user. The pattern determination policy may compute an average, mean, or other statistic of electronic activity for each time frame within a respective time interval in the time period. In some embodiments, the pattern determination policy may filter one or more data outliers in the temporal distribution (e.g., a number of electronic activities exceeding a standard deviation from the average or mean of electronic activity within a respective time frame). In some embodiments, the pattern determination policy may be used by the pattern determination engine to filter electronic activities within a respective time frame having a mean or average less than one electronic activity.

The method 4500 may include determining a time zone (4510). In some implementations, the system 200 may determine the time zone based on one or more temporal distributions or electronic activity patterns of the node profile. For example, the system 200 may compare at least one temporal distribution for a node profile to one or more other temporal distributions stored in a database or data structure and associated with a respective time zone. The database may include electronic activity temporal distributions corresponding to each of a plurality of time zones (e.g., 24 temporal distributions each corresponding to a respective time zone). In some implementations, the stored temporal distributions may be referred to as temporal distribution templates. The system 200 may identify the temporal distribution template included in the database that most closely matches at least one temporal distribution of the user. For example, the system 200 may identify which temporal distribution template of the database most closely matches the temporal distribution of the user based on which temporal distribution template tracks the temporal distribution of the user, which temporal distribution template has or includes upticks and downticks in electronic activity within the same time frame as the temporal distribution of the user, and so forth. When a match is determined, the system 200 may infer that the time zone for the user is the same as the time zone of the matching temporal distribution template, based on the match.

In some implementations, the temporal distribution templates can be generated or selected based on electronic activities associated with node profiles having known time zone information. For example, to generate a temporal distribution template for a given time zone, the system 200 may identify at least one node profile that is known to be associated with a user in that time zone. The system 200 may then access a set of electronic activities for the identified node profile to determine at least one temporal distribution of electronic activities for the node profile, in a manner similar to that described above, and the at least one temporal distribution may be stored as a temporal distribution template for that time zone. In some implementations, the system 200 may store more than one temporal distribution template for a given time zone. For example, the system 200 may store a respective temporal distribution template for each of a plurality of different industries, job titles, or any other characteristics of node profiles. In some implementations, typical electronic activity patterns may vary across industries or job titles, even within the same time zone. Thus, in order to infer a time zone of a user based on the user's electronic activities, it may be useful to compare the user's temporal distribution of electronic activities to a corresponding temporal distribution template representing one or more node profiles of users in the same industry or with the same job title. To achieve this, the system 200 may generate and store temporal distribution templates for different job titles, industries, or any other criteria within a given time zone. To infer a time zone for a particular node profile, the system 200 may select one or more temporal distribution templates to compare with the temporal distribution of the node profile based on match between an industry or job title of the node profile and a corresponding industry or job title of the one or more temporal distribution templates.

In some implementations, additional information can be used to further confirm or corroborate the user's inferred time zone. For example, the method 4500 can include parsing at least one electronic activity of the set of electronic activities to identify a character string corresponding to a geographic location. The geographic location can be or can include a residential address, a business address, a city, a state, a country, a zip code, or the like. The geographic location can be extracted, for example, from the body of an electronic activity. In some implementations, the location can be extracted from a signature block of an electronic activity corresponding to an email. The time zone can be determined based in part on the geographic location. For example, the geographic location can be matched to a corresponding time zone that the geographic location is positioned in. Thus, the system 200 can increase or decrease a confidence level associated with the inferred time zone, based on the geographic location extracted from the electronic activity.

The method 4500 may include storing an association between the time and the node profile (4512). In some embodiments, the system 200 may store an association between the time zone and the node profile in one or more data structures. The one or more data structures can be maintained by the data processing system 9300 or the system 200. The system 200 store an association between the node profile of the user and the identified time zone of the user. The system 200 may store the association in a database, memory, server, or other data structure. In some embodiments, the time zone may be static (e.g., once the time zone is identified, it remains the same). In some embodiments, the identified region may be dynamic (e.g., the time zone may be changed over time). For example, in some embodiments, the time zone for the node profile may be updated at various points in time. The system 200 may update the time zone for the node profile on a periodic basis (e.g., once every week, once every month, once every six months, once every year, etc.). The system 200 may update the time zone in a manner similar to that described above in connection with steps 4502-3010, for example to determine whether or when a person's time zone changes.

In some implementations, a change in time zone may correspond to an event such as travel or vacation for a user associated with the node profile. For example, the system 200 may detect an event condition. The system 200 may detect an event condition by identifying a second set of electronic activities engaged in by the participant corresponding to the node profile within a second time period (e.g., similar to 4504). The system 200 may generate a temporal distribution of electronic activity for the time interval in the second time period (e.g., similar to 4508). The system 200 may compare the temporal distribution to temporal distributions of the user for other time periods (or to an electronic activity pattern for the user based on multiple time periods) to determine that the temporal distribution satisfies an event condition detection policy for detecting an event condition. The system 200 may store an association between the node profile and the event condition in one or more data structures.

In some embodiments, the event condition detection policy can be used by the system to detect the event condition from a plurality of event conditions based on the determined electronic activity pattern satisfying a corresponding characteristic of a plurality of characteristics associated with a respective event condition. The event condition detection engine may access a plurality of characteristics associated respective labels for corresponding event conditions. For instance, when the user takes a vacation, a temporal distribution for a vacation period may include less than a threshold number of electronic activities engaged in by the user (e.g., zero or close to zero electronic activities). The event condition detection engine may access a characteristic associated with the condition corresponding to a vacation day (e.g., average electronic activities or number of electronic activities engaged in by a given user falling below a threshold). The event condition detection engine may detect an event condition corresponding to a vacation day based on the temporal distribution satisfying the corresponding characteristic associated with the vacation day event condition. In some implementations, the total number of the user's electronic activities may not substantially increase or decrease, but may instead be time shifted relative to the user's electronic activities during a different time period. For example, this may indicate that the user has traveled to a different time zone.

In some embodiments, the event condition detection policy includes a machine learning model trained to classify temporal distributions of electronic activity. The system 200 may use the machine learning model to determine that the temporal distribution satisfies the event condition detection policy. In some embodiments, the machine learning model may be trained to identify several event condition types. The machine learning model may use a first set of training examples including temporal distributions corresponding to a first event condition type and a first label identifying the first event condition type. The machine learning model may use a second set of training examples including temporal distributions corresponding to a second event condition type and a second label identifying the first event condition type. The system 200 may train the machine learning model using the first and second set of training examples. Following training, the machine learning model may detect the first and second event conditions based on traits, characteristics, and so forth of the temporal distributions of the user for a time interval. Continuing the examples described above, the machine learning model may be trained to detect event conditions corresponding to sick days and vacation days. For instance, the machine learning model may be trained with a set of training examples corresponding to a first event condition (e.g., travel to a new time zone) using a plurality sample temporal distributions corresponding to the first event condition (e.g., the new time zone). Each sample temporal distribution may be tagged or labeled with the respective event condition. Continuing this example, where a person is taking a vacation and traveling to a new time zone, the overall number of electronic activity engaged in by the person may decrease and/or the electronic activity temporal distribution may be time shifted relative to the person's typical time zone. The machine learning model may be trained with temporal distributions corresponding to the travel such that, where a temporal distribution corresponding to period of travel to a new time zone is provided as an input to the machine learning model, the machine learning model is configured to generate a label (or a confidence score corresponding to the label) identifying the temporal distribution as travel to a new time zone. In some implementations, the machine learning model may further identify a purpose for the travel (e.g., whether the travel is for vacation or for work).

The event condition detection engine may store an association between the node profile of the user and the event condition. The event condition detection engine may store the association between the node profile and event condition to more accurately reflect the user's location or current time zone. The event condition detection engine may store the association between the node profile 4304 and event condition and in association with the region of the time interval corresponding to the user's work schedule.

In some implementations, the method 4500 can include generating, by the system 200, a notification for a user associated with the node profile. For example, the notification can be any message or alert to be communicated to the user, such as an instruction or recommendation to perform a certain action (e.g., a work-related task). In some implementations, the system 200 can schedule transmission of the notification to a computing device of the user associated with the node profile based on the determined time zone. For example, it may be desirable to provide the notification to the user at a time (or during a window of time) when the user is likely to be able to receive, view, or act on the notification, such as during daytime hours or during typical work hours in the user's time zone. If the notification is instead sent during different hours (e.g., during the night), the user may be less likely to receive, view, or act on the notification. Thus, in some implementations, the system 200 can be configured to identify one or more restricted notification periods based on the determined time zone. The restricted notification periods may be any periods that are determined to be periods during which the user is unlikely to receive, view, or act on a notification, such as during the early morning or night time hours in the user's time zone. In some the system 200 can restrict transmission of the notification to the computing device of the user associated with the node profile during the restricted notification period.

### 28. Determining Domain Names of a Group Entity Using Electronic Activities and Systems of Record

An entity such as a company, a business, an enterprise, an organization, or any other entity having one or more members can be referred to as a group entity. In some implementations, a group entity can be represented by a node in a node graph. A node representing a group entity can be referred to as a group node. Members of the entity (e.g., employees) can also be represented by nodes, which can sometimes be referred to as member nodes. Information relating to nodes (e.g., group nodes or member nodes) can be stored in respective node profiles, for example in the form of node field-value pairs. A group node profile representing a group entity can include one or more field-value pairs corresponding to domain names for the group entity.

In some embodiments, a domain name for a group entity can refer to any label for one or more network resources associated with the group entity. For example, a domain name can be a label used to access an Internet web page owned by, hosted by, or otherwise affiliated with the group entity. Group entities can maintain domains for a variety of purposes, such as to provide customers, the public, or their employees or members with access to entity-related information hosted on web pages associated with the domain name. In some instances, a domain name may be or may include a name of the group entity itself. For example, a company whose corporate name is “XYZ Inc.” may be associated with the domain name “xyz.com.” However some group entities can also maintain or be associated with other domain names. In some implementations, a group entity may maintain other domain names having a hierarchical relationship with a primary domain name, which may be indicated by a prefix or a suffix in connection with a primary or top level domain name. For example, a group entity associated with the domain name xyz.com may also be associated with domain names such as sales.xyz.com or xyx.com/sales. In some implementations, a group entity may also maintain different domain names to serve users in different locations. For example, a group entity may maintain a group of related domain names such as xyz.com, xyz.co.uk, xyz.ca, etc.

In addition, a group entity may maintain or may be associated with domain names that may be unrelated to a name of the group entity. For example, in some implementations a first company may merge with, acquire, or be acquired by a second company. Prior to such a merger or acquisition, each company may maintain separate domain names (e.g., domain names corresponding to their corporate names). However, in some implementations one or both of the companies may continue to use their original domain name in addition to the domain name of the other company. As a result, a group entity may be associated with any number of domain names, some of which may have hierarchical relationships with one another, and some of which may be or may appear to be unrelated to one another. Furthermore, the domain names with which a group entity is associated may change over time. Thus, it can be technically challenging to accurately determine the set of domain names associated with a particular group entity.

To address these technical challenges of maintaining and updating accurate records of domain names that are associated with a particular group entity, a data processing system can parse electronic activities or record objects to determine a set of domain names that are associated with the group entity. The record object manager can identify tenant record objects that match with a master record object. The data processing system can maintain a plurality of node profiles that identify a group entity. The data processing system can identify a first domain name from a first set of electronic account identifiers of the plurality of node profiles. The data processing system can access a plurality of data points corresponding to a subset of the plurality of node profiles, such as electronic activities or record objects that are associated with some of the node profiles. Based on the electronic activities or record objects, the data processing system can determine at least a second domain name associated with the group entity. Then, the data processing system can store an association between the group entity, the first domain name, and the second domain name. These and other aspects of this disclosure are described further below.

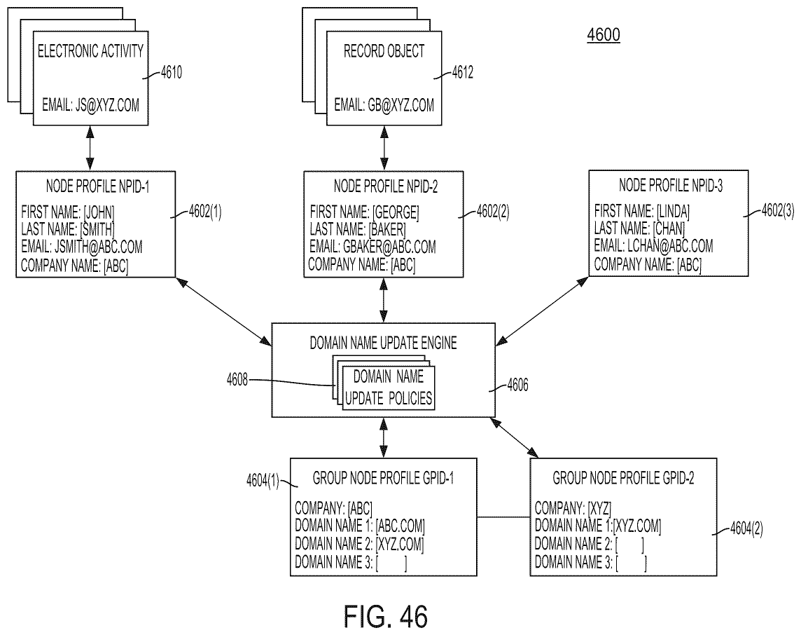

Referring now to FIG. 46, depicted is a block diagram of a system 4600 for determining domain names of a group entity using electronic activities and systems of record. In some implementations, the system 4600 can be or can include various components described above in connection with FIGS. 3, 4, and 16. For example, the system 4600 can be included within or can be implemented by at least one component of the node graph generation system 200 of FIG. 4. The system 4600 can include a domain name update engine 4606, which may access, receive, or maintain a set of node profiles. For example, FIG. 46 depicts three node profiles 4602(1)-4602(3) (generally referred to as node profiles 4602), as well as two group node profiles 4604(1) and 4604(2) (generally referred to as group node profiles 4604). The node profiles 4602 may be members of the group entity represented by the group node profile 4604(1). It should be understood that, while FIG. 46 depicts three node profiles 4602, in some implementations any number of node profiles 4602 may be maintained by the system 4600 in connection with the group node profile 4604(1). Similarly, the system 4600 may maintain additional group node profiles not depicted, as well as any number of member node profiles for each group node profile.

The domain name update engine 4606 can be configured to update the group node profiles 4604 to include domain names associated with the group entities represented by the group node profiles 4604. In some implementations, the domain name update engine 4606 can determine a domain name for a group node profile 4604 based on information contained in the corresponding member node profiles 4602. For example, each node profile 4602 includes a plurality of field-value pairs, such as a first name field, a last name field, an email field, and a company name field, as well as a corresponding value for each field. It should be appreciated that the fields depicted in FIG. 46 are illustrative only, and that the node profiles 4602 may include more, fewer, or different field-value pairs than are depicted in FIG. 46. In this example, each of the node profiles 4602 includes a respective email address ending with “abc.com.” In some implementations, the domain name update engine 4606 can extract the domain name abc.com from these email addresses and can update the group node profile 4604(1) to include the extracted domain name.

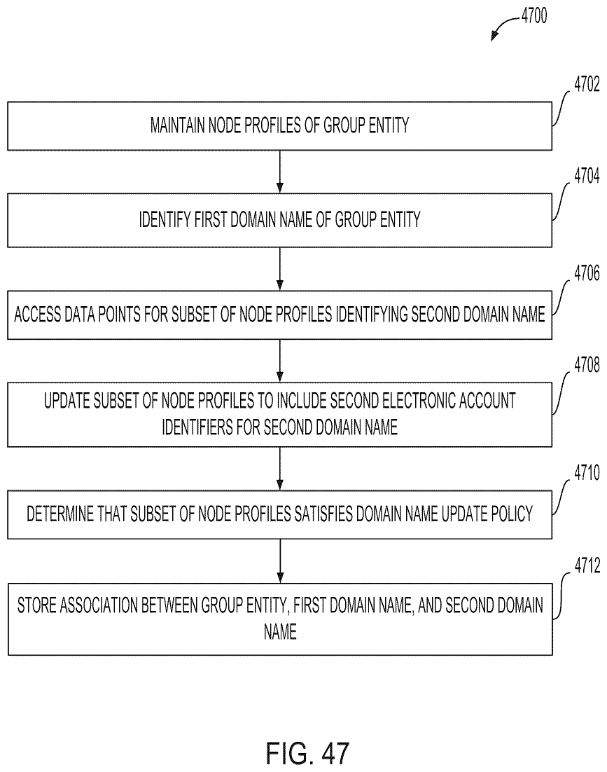

In some implementations, one or more of the node profiles 4602 may also be associated with one or more other domain names, and the domain name update engine 4606 can be configured to determine the other domain names, for example based on the electronic activities 4610 or record objects 4612 linked with the node profiles 4602. A method for doing so is shown and described in connection with FIG. 47.

Referring now to FIG. 47, depicted is a flow diagram of a method 4700 for determining domain names of a group entity using electronic activities and systems of record. The operations of method 4700 presented below are intended to be illustrative only. In some embodiments, method 4700 may be accomplished with one or more additional operations not described, and/or without one or more of the operations shown and discussed. Additionally, the order in which the operations of method 4700 are illustrated in FIG. 47 and described below is not intended to be limiting, and may vary from that shown in FIG. 47 in some embodiments. In some embodiments, method 4700 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 4700 in response to instructions stored electronically on an electronic storage medium. The one or more processing devices may include one or more devices configured through hardware, firmware, and/or software to be specifically designed for execution of one or more of the operations of method 4700. For example, in some embodiments at least some of the acts of the method 4700 can be performed by or in connection with the system 4600 of FIG. 46. FIGS. 46 and 47 are therefore described together below.

Referring again to FIG. 47, the method 4700 can include maintaining, by a data processing system, a plurality of node profiles corresponding to a group entity (BLOCK 4702). In some implementations, each node profile 4602 can be a set of information relating to a particular entity. For example, each node profile 4602 include information relating to a member of the group entity represented by the group node profile 4604(1). In instances in which the group entity is a business, the node profiles 4602 can represent employees of the business. Each node profile 4602 can store information in a plurality of field-value pairs. Each field may identify a particular type of information represented by the field. Each value may include one or more value data structures storing data points that support a particular value for the respective field. For example, the node profiles 4602 can include field-value pairs corresponding to a first name, a last name, an electronic identifier (e.g., an email address, a phone number, etc.), a company name (e.g., an employer), and the like.

The method 4700 can include identifying a first domain name of a group entity (BLOCK 4704). In some implementations, the domain name update engine 4606 can determine the first domain name based on information included in the node profiles 4602 that correspond to the group entity. For example, the node profiles 4602 may include field-value pairs for an electronic identifier such as an email address. The domain name update engine 4606 can parse one or more of the email addresses to determine the first domain name associated with the group entity. In some implementations, the domain name update engine 4606 can implement a matching technique using regular expressions to extract a domain name from an email address. For example, the domain name update engine 4606 can parse the email address to identify a predetermined character, such as “@,” which may be known to denote a domain name within the email address. Thus, the domain name update engine 4606 may discard, delete, or remove any characters of the email address occurring before the “@” character, as well as the “@” character itself. The domain name update engine 4606 can identify the remaining characters of the email address as the first domain name. For example, for the node profile 4602(1) corresponding to a user named John Smith, the domain name update engine 4606 can identify the email address as “jsmith@abc.com.” The domain name update engine 4606 can remove the characters preceding and including the “@” character to determine the first domain name as “abc.com.”

In some implementations, the domain name update engine 4606 can process information relating to more than one electronic account identifier within the set of node profiles 4602 in order to determine the first domain name. For example, the domain name update engine 4606 can extract a domain name from multiple node profiles 4602, and can confirm that at least a threshold number of the extracted domain names match one another before identifying the matching domain names as the first domain name identifier for the group entity. As described herein, the domain name update engine 4606 or the data processing system 9300 can be configured to maintain a value data structure for each value of a domain name field in a node profile of the group entity. The value data structure can be updated with entries identifying the data point (for instance, electronic activity or record object) that confirms the value or can serve as evidence of the value. The entries can have a contribution score based on a trust score of the source of the data point point and a time associated with when the data point was transmitted, received, last updated or generated, among others. The contribution scores can be combined to generate a confidence score of the value. The confidence score of the value can help to ensure that erroneous values or values occurring infrequently in field-value pairs corresponding to electronic account identifiers of the node profiles 4602 are not mistakenly identified as associated with the group entity, as such values may be more likely to be associated only with the node profiles in which they appear, rather than with the group entity itself.

The method 4700 can include accessing data points corresponding to a subset of the plurality of node profiles 4602 (BLOCK 4706). Each data point can identify a respective second electronic account identifier of a respective node profile 4602 of the subset. For example, each second electronic account identifier may correspond to a second email address of its respective node profile 4602. In some implementations, a node profile may be associated with more than one electronic account identifier for a variety of reasons. For example, a person represented by a node profile 4602 may be associated with multiple group entities and may have a separate electronic account identifier (e.g., email address) for each of the entities. In some other implementations, a person's email address may become inactive or invalid, and the person may be assigned a new email address having a different domain name. Thus, each respective second electronic account identifier of the plurality of data points can have a second domain name, different from the first domain name identified in BLOCK 4704.

In some implementations, at least some of the plurality of data points can include electronic activities, such as emails. For example, these may correspond to the electronic activities 4610 depicted in FIG. 46. The domain name update engine 4606 can access the electronic activities 4610 and can parse the electronic activities 4610 to determine the second electronic account identifiers. For example, the second electronic account identifiers can be a sender email address or a recipient email address. In some implementations, the domain name update engine 4606 can be configured to determine that plurality of data points (e.g., electronic activities) are associated with at least a subset of the node profile 4602 that correspond to the group entity. For example, in the example of FIG. 46, the domain name update engine 4606 can determine that the electronic activities 4610 are linked with the node profile 4602(1). In some implementations, the domain name update engine 4606 can determine that the electronic activities 4610 are linked with the node profile 4602(1) by matching activity field-value pairs included in the electronic activities 4610 with corresponding node field-value pairs included in the node profile 4602(1). For example, while the email address field-value pair of the electronic activities 4610 (e.g., js@xyz.com) may not match the email address field-value pair of the node profile 4602(1) (e.g., jsmith@abc.com), the domain name update engine 4606 may determine a match based on other criteria, such as a first name and last name field-value pairs that match across the electronic activities 4610 and the node profile 4602(1).

In some implementations, at least some of the plurality of data points can include record objects. For example, these may correspond to the record objects 4612 depicted in FIG. 46. In some implementations, the domain name update engine 4606 can access the record objects 4612 from a system of record associated with the record objects 4612. For example, the system of record can be a system of record owned or maintained by an external data source provider. In some other implementations, the system of record can be a system of record maintained by the system 4600. For example, the system of record can be a shadow system of record maintained by the system 4600 and intended to match with a corresponding system of record maintained by an external data source provider. The domain name update engine 4606 can access the record objects 4612 and can parse the record objects 4612 to determine the second electronic account identifiers, which may correspond to email addresses.

In some implementations, the domain name update engine 4606 can determine that the record objects 4612 are linked with the node profile 4602(2). The domain name update engine 4606 can determine that the record objects 4612 are linked with the node profile 4602(2) by matching object field-value pairs included in the record objects 4612 with corresponding node field-value pairs included in the node profile 4602(2). For example, while the email address field-value pair of the electronic activities 4610 (e.g., gb@xyz.com) may not match the email address field-value pair of the node profile 4602(2) (e.g., gbaker@abc.com), the domain name update engine 4606 may determine a match based on other criteria, such as a first name and last name field-value pairs that match across the record objects 4612 and the node profile 4602(2).

It should be understood that, while the electronic activities 4610 are linked with the node profile 4602(1) and the record objects 4612 are linked with the node profile 4602(2) in FIG. 46, the domain name update engine 4606 can be configured to access the plurality of data points corresponding to any electronic activity or any record object linked to any of the node profiles 4602. In some implementations, after accessing the plurality of data points, the domain name update engine 4606 can determine the second domain name based on the second electronic account identifiers associated with the data points. For example, the domain name update engine 4606 can extract the second domain name from the second electronic account identifiers in a manner similar to that described above for extracting the first domain name from the first electronic account identifiers. Thus, in the example of FIG. 46, the domain name update engine 4606 can determine the second domain name as “xyz.com,” based on the email addresses included in the electronic activities 4610 and the record objects 4612.

The method 4700 can include updating each respective node profile 4602 of the subset to include the respective second electronic account identifier corresponding to the node profile 4602 (BLOCK 4708). In some implementations, the domain name update engine 4606 can update a node profile 4602 to include a second electronic account identifier by adding an entry to a data structure corresponding to a value of an electronic account identifier field of the node profile 4602. For example, the entry can link the respective electronic activity 4610 or record object 4612 from which the second account identifier was extracted with the node profile 4602 to which the entry is added. In some implementations, the domain name update engine 4606 can also update a confidence score of either or both of the first electronic account identifier or the second electronic account identifier of any of the node profile 4602 that are updated to include a second electronic account identifier. For example, the update to the confidence score can be based on factors including a recency of the electronic activity or record object that triggered the update or a reliability of a data source provider who provided access to the electronic activity 4610 or record object 4612 that triggered the update. In some implementations, the domain name update engine 4606 can determine which node profiles 4602 should be updated to include respective second electronic account identifiers based on the linking of each electronic activity 4610 or record object 4612 with a respective node profile 4602, as described above. Thus, in the example of FIG. 46, the domain name update engine 4606 can update the node profile 4602(1) to include a value of “js@xyz.com” for an electronic identifier field, and can update the node profile 4602(2) to include a value of “gb@xyz.com” for an electronic identifier field. In some implementations, the original email addresses (e.g., jsmith@abc.com and gbaker@abc.com) may also remain stored in their respective node profiles 4602.

The method 4700 can include determining that the subset of the plurality of node profiles that was updated to include the respective second electronic account identifiers satisfies a domain name update policy (BLOCK 4710). For example, the domain name update engine 4606 can store, maintain, or otherwise access one or more domain name update policies 4608. A node profile 4602 may include any set of rules, heuristics, or any other criteria to determine whether the updating of the subset of node profiles in BLOCK 4708 should trigger a corresponding update to the group node profile 4604(1) to include the second domain name (e.g., xyz.com). For example, there may be instances in which the domain name update engine 4606 updates the subset of node profiles 4602 to include the second electronic account identifiers, but determines that the associated second domain name does not correspond to the group node profile 4604(1). In some implementations, the second electronic account identifiers may include personal email addresses that are not associated with the group node profile 4604(1). Thus, the domain name update policies 4608 may include a list of domain names corresponding to personal email services (e.g., gmail.com, hotmail.com, etc.) that may be shared across a subset of the node profiles 4602 but nevertheless should not be identified as corresponding to the group node profile 4604(1). The domain name update engine 4606 can compare the second domain name to such a list using the domain name update policies 4608 to determine whether the domain name update policy is satisfied.

In some implementations, a domain name update policy 4608 can be based on an update time interval. For example, the domain name update engine 4606 can select an update time interval, which may be specified as a time period of a predetermined duration (e.g., one day, one week, one month, etc.) or a time period between a predetermined start time and a predetermined end time (e.g., a first date and a second date). In order to satisfy the node profile 4602, the domain name update engine 4606 can determine whether the subset of the plurality of node profiles 4602 that was updated to include the respective second electronic account identifiers was updated during the update time interval (e.g., not outside of the update time interval). For example, if all of the node profiles 4602 of the subset were updated within a predetermined time frame, it may be more likely that a domain name associated with the subset of node profiles is also associated with the group node profile 4604(1). In some implementations, this could be a result of a first company (e.g., ABC) merging with a second company (XYZ), thereby triggering simultaneous or substantially simultaneous issuance of a batch of new email addresses for the xyz.com domain name to be issued to the subset of node profile 4602s.

The method 4700 can include storing an association between the group entity, the first domain name, and the second domain name (BLOCK 4712). In some implementations, the domain name update engine 4606 can store the association in one or more data structures. For example, in some implementations the domain name update engine 4606 can store the association in the group node profile 4604(1) or in one or more underlying data structures used to implement the group node profile 4604(1). Thus, as depicted in FIG. 46, the group node profile 4604(1) can store a first domain name (e.g., abc.com) and a second domain name (e.g., xyz.com).

In some implementations, the association between the group node profile 4604(1), the first domain name, and the second domain name may also be indicative of a relationship between the group entity represented by the group node profile 4604(1) and another group entity, such as the group entity represented by the group node profile 4604(2). For example, as described above, the system domain name update engine 4606 may associate the node profile 4604(1) with the second domain name xyz.com after two companies represented by the node profiles 4604 merge. Thus, in some implementations, the method 4700 can also include identifying or determining a relationship between the group node profile and a second group node profile. The domain name update engine 4606 can identify the relationship by accessing one or more electronic activities sent or received by the subset of the plurality of node profiles 4602 that were updated in BLOCK 4708, such as the electronic activities 4610. The domain name update engine 4606 can parse the electronic activities 4610 to identify a relationship between the two group entities. For example, the domain name update engine 4606 can identify key words or phrases that may indicate a relationship (e.g., “merger,” “acquisition,” etc.). In some implementations, the domain name update engine 4606 can parse the electronic activities 4610 using natural language processing techniques to determine a newly formed relationship between the two group entities. If the domain name update engine 4606 determines a relationship, an association between the two group entities can be stored in one or more data structures.

In some implementations, a relationship between the two group entities can be a hierarchical relationship. For example, the relationship can indicate that one of the group entities has become a subsidiary or a parent of the other group entity. In some implementations, may be desirable to record such hierarchical information. Thus, the method 4700 may also include determining whether the relationship between the first group entity and the second group entity satisfies a hierarchy update policy. For example, the hierarchy update policy can be any set of rules or heuristics to determine whether the hierarchical relationship between the group entities is sufficient to warrant being recorded. In some implementations, the domain name update engine 4606 can determine that the relationship between two group entities satisfies a hierarchy update policy only if there is a formal tie or link in a corporate structure between the two group entities, such as when one of the group entities is an owner, a parent, a child, or a subsidiary of the other. Other (e.g., less direct) relationships may not satisfy the hierarchy update policy. For example, if the relationship is a buyer-seller relationship or other type of client or customer relationship, rather than a hierarchical corporate relationship, the domain name update engine 4606 may determine that the hierarchy update policy is not satisfied.

In some implementations, if the domain name update engine 4606 determines that the hierarchy relationship policy is satisfied, the domain name update engine 4606 can update a node graph including group nodes for each of the group entities (e.g., the group node profile 4604(1) and the group node profile 4604(2)) to include a new or updated connection between the group entities. In some implementations, updating the node graph to include a new connection can include adding a new edge to the node graph. The new edge can link the group node profile 4604(1) and the group node profile 4604(2). For example, as depicted in FIG. 46, the edge can be represented by a line joining the group node profile 4604(1) and the group node profile 4604(2).

In some implementations, the domain name update engine 4606 can also be configured to update at least one of a record object or an account hierarchy data structure to indicate the relationship between the two group entities, responsive to determining that the relationship satisfies the hierarchy update policy. For example, the domain name update engine 4606 can identify a record object that corresponds to one or both of the group node profile 4604(1) and the group node profile 4604(2), such as the record objects 4612. In some implementations, such a record object 4612 may include or may be linked with a hierarchy data structure indicating the relationship between the group node profile 4604(1) and the group node profile 4604(2). For example, a record object 4612 may be an opportunity record object corresponding to an opportunity for an entity (e.g., a third party entity not depicted in FIG. 46) that may be interested in selling a product or service to the group entity associated with the group node profile 4604(1). The domain name update engine 4606 can update the record object 4612 (or a hierarchy data structure linked to the record object 4612) to indicate the newly inferred relationship between the group node profile 4604(1) and the group node profile 4604(2), thereby informing the third party entity that their sales prospect corresponding to the group node profile 4604(1) is also associated with another group entity (i.e., the group entity represented by the group node profile 4604(2)). For example, one of the record objects 4612 may have an object field-value pair corresponding to the first domain name “abc.com,” and the domain name update engine 4606 may update the record object 4612 (or may send an instruction to a data source provider that maintains the record object 4612) to add a value corresponding to the second domain name “xyz.com” as well.

In some implementations, the method 4700 can also include updating information stored in the node profiles 4602, based on the inferred relationship between the group node profiles 4604 and their respective domain names. For example, after a node profile 4602 has been updated to include the second electronic account identifier (BLOCK 4708), the domain name update engine 4606 can monitor additional electronic activities to determine whether the first electronic account identifier remains active for that node profile 4602. In some implementations, the second electronic account identifier may entirely replace the first electronic account identifier, for example if the person is assigned the second electronic account identifier in response to a merger that renders the first electronic account identifier obsolete or deactivated. To detect such a change, the domain name update engine 4606 may access a “bounceback” electronic activity for the node profile 4602, which may be any electronic activity automatically generated by a corresponding electronic account to indicate that the electronic activity is undeliverable. Typically, a bounceback electronic activity can be received in response to sending an email or other electronic activity to an invalid or deactivated email address. Thus, after receiving a bounceback electronic activity for a node profile 4602, the domain name update engine 4606 can identify the electronic activity that triggered the bounceback (e.g., the email that was sent to the invalid or deactivated email address), and can decrease a confidence score of a value corresponding to that destination email address in the node profile 4602. For example, if the email that triggered the bounceback was sent to an email address of the first domain name, the domain name update engine 4606 can decrease a confidence score associated with the email address of the first domain.

In some implementations, the domain name update engine 4606 can also infer patterns relating to electronic account identifiers of the node profiles 4602, which may be associated with either or both of the first domain name and the second domain name. For example, upon receiving one or more electronic activities 4610 linked with the node profile 4602(1), the domain name update engine 4606 can correlate node field-value pairs of the node profile 4602(1) with the second electronic account identifier (e.g., email address) of the electronic activities 4610. A correlation can be used to identify a pattern for the second electronic account identifier. For example, in some implementations the domain name update engine 4606 can correlate values of node field-value pairs corresponding to a first name, a last name, or any other node-field value pairs of the node profile 4602(1) with an email address included in the electronic activities 4610. In the example of FIG. 46, the domain name update engine 4606 may determine that the pattern for assigning the local-part (e.g., the characters preceding the “@” character) of an email addresses for the second domain name xyz.com is to concatenate the user's first initial with the user's last initial. Thus, the email address for John Smith on the second domain xyz.com is “js@xyz.com.”

In some implementations, the domain name update engine 4606 can store an association between the pattern identified for assigning electronic account identifiers and either or both of the group node profile 4604(1) or the group node profile 4604(2). The domain name update engine 4606 can then refer to stored pattern in order to generate or predict an electronic account identifier with the second domain name for a user even before accessing an electronic activity corresponding to an electronic account identifier for the second domain name for that person. Thus, in the example of FIG. 46, the domain name update engine 4606 can use the pattern determined for assigning email addresses for the second domain name xyz.com to generate or predict an email address on that domain for the user corresponding to the node profile 4602(3), even though no electronic activities have been received to specify the user's email address on the second domain. For example, based on the user's first name “Linda” and last name “Chan,” the domain name update engine 4606 can apply the pattern to concatenate the user's first initial and last initial to generate or predict the email address “lc@xyz.com.” In some implementations, the domain name update engine 4606 can then update the node profile 4602(3) to include a value corresponding to this generated or predicted email address.

The domain name update engine 4606 can also use an inferred pattern for assigning email addresses to match an electronic activity with a corresponding node profile 4602. For example, when a new electronic activity such as an email is received, the domain name update engine 4606 can parse an electronic account identifier (e.g., sender or recipient email address) based on the determined pattern. In some implementations, the domain name update engine 4606 may be able to determine at least one of a first initial, a last initial, a first name, or a last name, based on the inferred pattern and the electronic account identifier. Therefore, the domain name update engine 4606 can determine an activity field-value pair for the electronic activity (e.g., a first name, a last name, etc.), which can be matched with a corresponding node field-value pair of a node profile 4602. In the example of FIG. 46, the domain name update engine 4606 may receive a new electronic activity having an email address of “lc@xyz.com,” and may identify the node profile 4602(3) as a matching node profile for the electronic activity, because the node profile 4602(3) includes a first name field with a value indicating a first initial of “1” and a last name field with a value indicating a last initial of “c.” Thus, the domain name update engine 4606 can use the inferred pattern to facilitate matching of electronic activities with corresponding node profiles 4602.

### 29. Computer System

Various operations described herein can be implemented on computer systems, which can be of generally conventional design. FIG. 48 shows a simplified block diagram of a representative server system 4800 and client computer system 4814 usable to implement certain embodiments of the present disclosure. In various embodiments, server system 4800 or similar systems can implement services or servers described herein or portions thereof. Client computer system 4814 or similar systems can implement clients described herein. Each of the systems 9300, 200 and others described herein can be similar to the server system 4800.

Server system 4800 can have a modular design that incorporates a number of modules 4802 (e.g., blades in a blade server embodiment); while two modules 4802 are shown, any number can be provided. Each module 4802 can include processing unit(s) 4804 and local storage 4806.

Processing unit(s) 4804 can include a single processor, which can have one or more cores, or multiple processors. In some embodiments, processing unit(s) 4804 can include a general-purpose primary processor as well as one or more special-purpose co-processors such as graphics processors, digital signal processors, or the like. In some embodiments, some or all processing units 4804 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 4804 can execute instructions stored in local storage 4806. Any type of processors in any combination can be included in processing unit(s) 4804.

Local storage 4806 can include volatile storage media (e.g., conventional DRAM, SRAM, SDRAM, or the like) and/or non-volatile storage media (e.g., magnetic or optical disk, flash memory, or the like). Storage media incorporated in local storage 4806 can be fixed, removable or upgradeable as desired. Local storage 4806 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 4804 need at runtime. The ROM can store static data and instructions that are needed by processing unit(s) 4804. The permanent storage device can be a non-volatile read-and-write memory device that can store instructions and data even when module 4802 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 4806 can store one or more software programs to be executed by processing unit(s) 4804, such as an operating system and/or programs implementing various server functions such as functions of the data processing system 9300 of FIG. 2, the node graph generation system 300, or any other system described herein, or any other server(s) associated with data processing system 9300 of FIG. 2 or the node graph generation system 300 or any other system described herein.

“Software” refers generally to sequences of instructions that, when executed by processing unit(s) 4804 cause server system 4800 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 4804. Software can be implemented as a single program or a collection of separate programs or program modules that interact as desired. From local storage 4806 (or non-local storage described below), processing unit(s) 4804 can retrieve program instructions to execute and data to process in order to execute various operations described above.

In some server systems 4800, multiple modules 4802 can be interconnected via a bus or other interconnect 4808, forming a local area network that supports communication between modules 4802 and other components of server system 4800. Interconnect 4808 can be implemented using various technologies including server racks, hubs, routers, etc.

A wide area network (WAN) interface 4810 can provide data communication capability between the local area network (interconnect 4808) and a larger network, such as the Internet. Conventional or other activities technologies can be used, including wired (e.g., Ethernet, IEEE 802.3 standards) and/or wireless technologies (e.g., Wi-Fi, IEEE 802.11 standards).

In some embodiments, local storage 4806 is intended to provide working memory for processing unit(s) 4804, providing fast access to programs and/or data to be processed while reducing traffic on interconnect 4808. Storage for larger quantities of data can be provided on the local area network by one or more mass storage subsystems 4812 that can be connected to interconnect 4808. Mass storage subsystem 4812 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 4812. In some embodiments, additional data storage resources may be accessible via WAN interface 4810 (potentially with increased latency).

Server system 4800 can operate in response to requests received via WAN interface 4810. For example, one of modules 4802 can implement a supervisory function and assign discrete tasks to other modules 4802 in response to received requests. Conventional work allocation techniques can be used. As requests are processed, results can be returned to the requester via WAN interface 4810. Such operation can generally be automated. Further, in some embodiments, WAN interface 4810 can connect multiple server systems 4800 to each other, providing scalable systems capable of managing high volumes of activity. Conventional or other techniques for managing server systems and server farms (collections of server systems that cooperate) can be used, including dynamic resource allocation and reallocation.

Server system 4800 can interact with various user-owned or user-operated devices via a wide-area network such as the Internet. An example of a user-operated device is shown in FIG. 48 as client computing system 4814. Client computing system 4814 can be implemented, for example, as a consumer device such as a smartphone, other mobile phone, tablet computer, wearable computing device (e.g., smart watch, eyeglasses), desktop computer, laptop computer, and so on.

For example, client computing system 4814 can communicate via WAN interface 4810. Client computing system 4814 can include conventional computer components such as processing unit(s) 4816, storage device 4818, network interface 4820, user input device 4822, and user output device 4824. Client computing system 4814 can be a computing device implemented in a variety of form factors, such as a desktop computer, laptop computer, tablet computer, smartphone, other mobile computing device, wearable computing device, or the like.

Processor 4816 and storage device 4818 can be similar to processing unit(s) 4804 and local storage 4806 described above. Suitable devices can be selected based on the demands to be placed on client computing system 4814; for example, client computing system 4814 can be implemented as a “thin” client with limited processing capability or as a high-powered computing device. Client computing system 4814 can be provisioned with program code executable by processing unit(s) 4816 to enable various interactions with server system 4800 of a message management service such as accessing messages, performing actions on messages, and other interactions described above. Some client computing systems 4814 can also interact with a messaging service independently of the message management service.

Network interface 4820 can provide a connection to a wide area network (e.g., the Internet) to which WAN interface 4810 of server system 4800 is also connected. In various embodiments, network interface 4820 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 4822 can include any device (or devices) via which a user can provide signals to client computing system 4814; client computing system 4814 can interpret the signals as indicative of particular user requests or information. In various embodiments, user input device 4822 can include any or all of a keyboard, touch pad, touch screen, mouse or other pointing device, scroll wheel, click wheel, dial, button, switch, keypad, microphone, and so on.

User output device 4824 can include any device via which client computing system 4814 can provide information to a user. For example, user output device 4824 can include a display to display images generated by or delivered to client computing system 4814. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 4824 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 4804 and 4816 can provide various functionality for server system 4800 and client computing system 4814, including any of the functionality described herein as being performed by a server or client, or other functionality associated with message management services.

It will be appreciated that server system 4800 and client computing system 4814 are illustrative and that variations and modifications are possible. Computer systems used in connection with embodiments of the present disclosure can have other capabilities not specifically described here. Further, while server system 4800 and client computing system 4814 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. For instance, although specific examples of rules (including triggering conditions and/or resulting actions) and processes for generating suggested rules are described, other rules and processes can be implemented. Embodiments of the disclosure can be realized using a variety of computer systems and communication technologies including but not limited to specific examples described herein.

Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method comprising:
identifying, by one or more processors, an electronic activity corresponding to a scheduled event and identifying a plurality of candidate participants;
accessing, by the one or more processors, for a first participant of the candidate participants, a plurality of electronic activities corresponding to an electronic account of the first participant;
extracting, by the one or more processors, for each electronic activity of the plurality of electronic activities, a feature set including one or more features based on participants included in the electronic activity or content included in a body of the electronic activity;
determining, by the one or more processors, for each electronic activity of the plurality of electronic activities, a relevancy score between the electronic activity and the scheduled event based on the extracted feature set;
identifying, by the one or more processors, a subset of electronic activities of the plurality of electronic activities that have respective relevancy scores that satisfy a first threshold value;
determining, by the one or more processors, a probability score indicating a likelihood that the first participant attended the scheduled event based on the subset of electronic activities;
generating, by the one or more processors responsive to the probability score satisfying a second threshold value, an attendance tag indicating that the first participant attended the scheduled event; and
storing, by the one or more processors, in one or more data structures, an association between the attendance tag and the electronic activity corresponding to the scheduled event.

2. The method of claim 1, further comprising:
determining, by the one or more processors, a confidence score for the attendance tag based on the probability score; and
assigning, by the one or more processors, the confidence score to the attendance tag.

3. The method of claim 2, further comprising:
accessing, by the one or more processors, a second electronic activity;
extracting, by the one or more processors, for the second electronic activity, a second feature set including one or more features based on participants included in the second electronic activity or content included in a body of the second electronic activity;
determining, by the one or more processors, based on the second feature set of the second electronic activity, that a relevancy score between the second electronic activity and the scheduled event satisfies the first threshold value; and
updating, by the one or more processors, the confidence score of the attendance tag based on the second electronic activity.

4. The method of claim 1, wherein the probability score indicating the likelihood that the first participant attended the scheduled event is a first probability score, the method further comprising:
determining, by the one or more processors, a second probability score indicating a likelihood that the scheduled event occurred, based on the subset of electronic activities;
generating, by the one or more processors, responsive to determining that the second probability score exceeds a predetermined event occurrence threshold, an occurrence tag indicating that the scheduled event occurred; and
storing, by the one or more processors, an association between the occurrence tag and the electronic activity corresponding to the scheduled event.

5. The method of claim 4, further comprising:
determining, by the one or more processors, a confidence score for the occurrence tag based on the second probability score; and
assigning, by the one or more processors, the confidence score to the occurrence tag.

6. The method of claim 5, further comprising:
parsing, by the one or more processors, the subset of electronic activities to identify a number of the candidate participants that attended the scheduled event; and
updating, by the one or more processors, the confidence score of the occurrence tag based on the number of the candidate participants that attended the scheduled event.

7. The method of claim 6, further comprising:
determining, by the one or more processors, an attendance rate of the scheduled event, based on the number of the candidate participants that attended the scheduled event;
generating, by the one or more processors, an attendance metric for the scheduled event; and
transmitting, by the one or more processors, data using the attendance metric to a computing device of an organizer of the scheduled event.

8. The method of claim 1, further comprising:
accessing, by the one or more processors, a performance profile of a node profile corresponding to the first participant; and
updating, by the one or more processors, the performance profile of the first participant responsive to the attendance tag.

9. The method of claim 1, further comprising:
parsing, by the one or more processors, the subset of electronic activities to identify one of an electronic access values associated with the scheduled event or a physical address associated with the scheduled event; and
determining, by the one or more processors, an event type of the scheduled event based on identifying the electronic access value or the physical address associated with the scheduled event.

10. The method of claim 1, further comprising determining, by the one or more processors, that the scheduled event corresponds to a first event type responsive to determining that the electronic activity associated with the scheduled event satisfies a first event type detection policy.

11. The method of claim 1, further comprising:
identifying, by the one or more processors, a first electronic activity of the subset of electronic activities of the first participant having a recipient identifier that matches a communication string included in the electronic activity corresponding to the scheduled event,
wherein generating the attendance tag further comprises generating, by the one or more processors, the attendance tag indicating that the first participant attended the scheduled event responsive to identifying the first electronic activity of the one or more electronic activities having the recipient identifier that matches the communication string included in the electronic activity corresponding to the scheduled event.

12. The method of claim 11, wherein the first electronic activity comprises an electronic voice call, the method further comprising:
determining, by the one or more processors, a duration of the electronic voice call,
wherein generating the attendance tag further comprises generating, by the one or more processors, the attendance tag indicating that the first participant attended the scheduled event responsive to determining that the duration of the electronic voice call satisfies a duration threshold.

13. The method of claim 13, further comprising:
parsing, by the one or more processors, at least one electronic activity of the subset sent or received within a predetermined time period of the scheduled event to determine a location associated with the at least one electronic activity;
comparing the location to a physical address included in the electronic activity corresponding to the scheduled event to determine a proximity between the location and the physical address,
wherein generating the attendance tag further comprises generating, by the one or more processors, the attendance tag indicating that the first participant attended the scheduled event responsive to determining that the proximity is within a predetermined proximity threshold.

14. The method of claim 13, wherein parsing the at least one electronic activity of the subset of electronic activities comprises extracting, by the one or more processors, from the at least one electronic activity, at least one of an internet protocol (IP) address of a computing device of the first participant or a GPS location of the first participant within the predetermined time period.

15. The method of claim 1, wherein the attendance tag comprises a first attendance tag, the method further comprising:
accessing, by the one or more processors, for a second participant of the candidate participants, a second plurality of electronic activities corresponding to a second electronic account of the second participant;
extracting, by the one or more processors, for each electronic activity of the second plurality of electronic activities, a second feature set including one or more features based on participants included in the electronic activity or content included in a body of the electronic activity;
determining, by the one or more processors, for each electronic activity of the second plurality of electronic activities, a second relevancy score between the electronic activity and the scheduled event based on the extracted feature set;
identifying, by the one or more processors, a second subset of electronic activities of the second plurality of electronic activities that have respective relevancy scores that satisfy a second threshold value;
determining, by the one or more processors, a second probability score indicating a likelihood that the second participant attended the scheduled event based on the second subset of electronic activities;
generating, by the one or more processors responsive to the probability score not satisfying the second threshold value, a second attendance tag indicating that the second participant did not attend the scheduled event; and
storing, by the one or more processors, in the one or more data structures, an association between the second attendance tag and the electronic activity corresponding to the scheduled event.

16. The method of claim 1, further comprising:
identifying, by the one or more processors, for each electronic activity of the plurality of electronic activities, a context of the electronic activity, wherein determining the relevancy score between the electronic activity and the scheduled event further comprises determining the relevancy score based on the context.

17. The method of claim 1, wherein the first electronic activity comprises an electronic voice call, the method further comprising:
receiving, by the one or more processors from a data source provider corresponding to a host of the scheduled event, at least one phone number that participated in the electronic voice call; and
matching, by the one or more processors, the at least one phone number that participated in the electronic voice call with a phone number of the first participant, wherein determining the probability score indicating the likelihood that the first participant attended the scheduled event further comprises determining the probability score based on the match.

18. The method of claim 1, wherein the first electronic activity comprises an electronic voice call, the method further comprising:
accessing, by the one or more processors, audio information corresponding to the electronic voice call;
identifying, by the one or more processors, a first acoustic fingerprint of an attendee of the electronic voice call, based on the audio information; and
matching, by the one or more processors, the first acoustic fingerprint of the attendee with a second acoustic fingerprint of the first participant, wherein determining the probability score indicating the likelihood that the first participant attended the scheduled event further comprises determining the probability score based on the match.

19. The method of claim 1, further comprising:
receiving, by the one or more processors from a data source provider, travel information for the first participant, the travel information corresponding to travel within a predetermined time period of the scheduled event;
extracting, by the one or more processors, a location from the travel information; and
comparing the location to a physical address included in the electronic activity corresponding to the scheduled event to determine a proximity between the location and the physical address,
wherein generating the attendance tag further comprises generating, by the one or more processors, the attendance tag indicating that the first participant attended the scheduled event responsive to determining that the proximity is within a predetermined proximity threshold.

20. The method of claim 1, further comprising:
determining, by the one or more processors, a second subset of the plurality of electronic activities that were transmitted by an electronic account of the first participant during the scheduled event; and
generating, by the one or more processors, a meeting engagement score based on a number of electronic activities included in the second subset.

21. The method of claim 20, further comprising:
determining, by the one or more processors from the electronic activities, a percentage of the second subset of electronic activities that are relevant to the event; and
adjusting, by the one or more processors, the engagement score based on the percentage.

22. A system comprising:
one or more hardware processors configured by machine-readable instructions to:
identify an electronic activity corresponding to a scheduled event and identifying a plurality of candidate participants;
access, for a first participant of the candidate participants, a plurality of electronic activities corresponding to an electronic account of the first participant;
extract, for each electronic activity of the plurality of electronic activities, a feature set including one or more features based on participants included in the electronic activity or content included in a body of the electronic activity;
determine, for each electronic activity of the plurality of electronic activities, a relevancy score between the electronic activity and the scheduled event based on the extracted feature set;
identify a subset of electronic activities of the plurality of electronic activities that have respective relevancy scores that satisfy a first threshold value;
determine a probability score indicating a likelihood that the first participant attended the scheduled event based on the subset of electronic activities;
generate, responsive to the probability score satisfying a second threshold value, an attendance tag indicating that the first participant attended the scheduled event; and
store, in one or more data structures, an association between the attendance tag and the electronic activity corresponding to the scheduled event.

23. The system of claim 22, wherein the one or more processors are further configured to:
determine a confidence score for the attendance tag based on the probability score; and
assign the confidence score to the attendance tag.

24. The system of claim 23, wherein the one or more processors are further configured to:
access a second electronic activity;
extract, for the second electronic activity, a second feature set including one or more features based on participants included in the second electronic activity or content included in a body of the second electronic activity;
determine, based on the second feature set of the second electronic activity, that a relevancy score between the second electronic activity and the scheduled event satisfies the first threshold value; and
update the confidence score of the attendance tag based on the second electronic activity.

25. The system of claim 22, wherein the probability score indicating the likelihood that the first participant attended the scheduled event is a first probability score, and wherein the one or more processors are further configured to:
determine a second probability score indicating a likelihood that the scheduled event occurred, based on the subset of electronic activities;
generate, responsive to determining that the second probability score exceeds a predetermined event occurrence threshold, an occurrence tag indicating that the scheduled event occurred; and
store an association between the occurrence tag and the electronic activity corresponding to the scheduled event.

26. A non-transitory computer-readable storage medium having instructions embodied thereon, the instructions being executable by one or more processors to perform a method comprising:
identifying an electronic activity corresponding to a scheduled event and identifying a plurality of candidate participants;
accessing, for a first participant of the candidate participants, a plurality of electronic activities corresponding to an electronic account of the first participant;
extracting, for each electronic activity of the plurality of electronic activities, a feature set including one or more features based on participants included in the electronic activity or content included in a body of the electronic activity;
determining, for each electronic activity of the plurality of electronic activities, a relevancy score between the electronic activity and the scheduled event based on the extracted feature set;
identifying a subset of electronic activities of the plurality of electronic activities that have respective relevancy scores that satisfy a first threshold value;
determining a probability score indicating a likelihood that the first participant attended the scheduled event based on the subset of electronic activities;
generating, responsive to the probability score satisfying a second threshold value, an attendance tag indicating that the first participant attended the scheduled event; and
storing, in one or more data structures, an association between the attendance tag and the electronic activity corresponding to the scheduled event.
